(19) United States
(12) Patent Application Publication
(10) Pub. No.: US 2007/0026505 A1
(43) Pub. Date: Feb. 1, 2007

(54) Amino acid and metabolite biosynthesis

(76) Inventors: Kevin T. Madden, Arlington, MA (US); Michael J. Walbridge, Dorchester, MA (US); Peter S. Yorgey, Cambridge, MA (US); Reed Doten, Framingham, MA (US)

Correspondence Address: FISH & RICHARDSON PC, P.O. BOX 1022, MINNEAPOLIS, MN, 55440-1022, US

(21) Appl. No.: 11/455,390

(22) Filed: Jun. 19, 2006

Related U.S. Application Data
(60) Provisional application No. 60/692,037, filed on Jun. 17, 2005.
(60) Provisional application No. 60/750,592, filed on Dec. 15, 2005.

(51) Int. Cl. C12P 13/04; C12N 1/21; C12N 15/74

(52) U.S. Cl. 435/106; 435/252.33; 435/252.3; 435/471

(57) ABSTRACT

Bacterial strains that are engineered to increase the production of amino acids, including aspartate-derived amino acids (e.g., methionine, lysine, threonine, isoleucine, and S-adenosylmethionine (S-AM)) and cysteine, and related metabolites are described. The strains can be genetically engineered to harbor one or more nucleic acid molecules (e.g., recombinant nucleic acid molecules) encoding a polypeptide (e.g., a polypeptide that is heterologous or homologous to the host cell) and/or they may be engineered to increase or decrease expression and/or activity of polypeptides (e.g., by mutation of endogenous nucleic acid sequences).

### RELATED APPLICATION INFORMATION

This application claims priority to U.S. provisional application Ser. No. 60/692,037, filed Jun. 17, 2005, and to U.S. provisional application Ser. No. 60/750,592, filed Dec. 15, 2005, both of which are herein incorporated by reference.

### SEQUENCE LISTING

This application incorporates by reference the sequence listing saved as an ASCII text file and identified as “14184-064001.txt”, containing 2,202 KB of data, and created on Sep. 21, 2006, filed in computer-readable format (CRF) and Official copy (Copy 1 and Copy 2), each encoded on CD-ROM.

### TECHNICAL FIELD

This disclosure relates to bacterial amino acid and metabolite biosynthesis, and more particularly to biosynthesis of methionine and related amino acids and metabolites.

### BACKGROUND

Industrial fermentation of bacteria is used to produce commercially useful metabolites such as amino acids, nucleotides, vitamins, and antibiotics. Many of the bacterial production strains used in these fermentation processes have been generated by random mutagenesis and selection of mutants (Demain, A. L. Trends Biotechnol. 18:26-31, 2000). Accumulation of secondary mutations in mutagenized production strains and derivatives of these strains can reduce the efficiency of metabolite production due to altered growth and stress-tolerance properties. The availability of genomic information for production strains and related bacterial organisms provides an opportunity to construct new production strains by the introduction of cloned nucleic acids into naïve, unmanipulated host strains, thereby allowing amino acid production in the absence of deleterious mutations (Ohnishi, J., et al. Appl Microbiol Biotechnol. 58:217-223, 2002). Similarly, this information provides an opportunity for identifying and overcoming the limitations of existing production strains.

### SUMMARY

Compositions and methods for the production of amino acids and related metabolites in bacteria are described herein. Bacterial strains that are engineered to increase the production of amino acids, including aspartate-derived amino acids (e.g., methionine, lysine, threonine, isoleucine, and S-adenosylmethionine (S-AM)) and cysteine, and related metabolites are described. The strains can be genetically engineered to harbor one or more nucleic acid molecules (e.g., recombinant nucleic acid molecules) encoding a polypeptide (e.g., a polypeptide that is heterologous or homologous to the host cell) and/or they may be engineered to increase or decrease expression and/or activity of polypeptides (e.g., by mutation of endogenous nucleic acid sequences). The expressed polypeptides, which can be expressed by various methods familiar to those skilled in the art, include variant polypeptides, such as variant polypeptides with reduced feedback inhibition. The variant polypeptides may exhibit reduced feedback inhibition by a product or an intermediate of an amino acid biosynthetic pathway, such as S-adenosylmethionine, lysine, threonine or methionine, relative to wild type forms of the proteins. Also described herein are variant polypeptides and bacterial cells genetically modified to contain the nucleic acids. Combinations of nucleic acids, and cells that harbor the combinations of nucleic acids, are also provided herein. Improved bacterial production strains, including, without limitation, strains of coryneform bacteria and Enterobacteriaceae (e.g., Escherichia coli (E. coli)) are also described.

Bacterial polypeptides that regulate the production of methionine and related amino acids and metabolites include, for example, polypeptides involved in the metabolism of methionine, aspartate, homoserine, cysteine, sulfur, folate, and vitamin B12. The polypeptides include enzymes that catalyze the conversion of intermediates of amino acid biosynthetic pathways to other intermediates and/or end products, polypeptides required for the import or export of precursors, cofactors, intermediates or end products, and polypeptides that regulate the expression and/or function of such enzymes and/or import/export regulators. Tables 1-6, below, list some, but not all of the relevant polypeptides. FIG. 1 schematically depicts the methionine biosynthesis pathway and indicates additional pathways that yield the precursors and cofactors used in the methionine biosynthesis pathway. These additional pathways are depicted in FIGS. 2-4. Additional polypeptides and variants useful for producing amino acids and metabolites are described below.

In various embodiments, the host bacterium has reduced activity of one or more polypeptides (e.g., a polypeptide involved in amino acid synthesis; e.g., an endogenous polypeptide with reduced activity relative to a control). Reducing the activity of particular polypeptides involved in amino acid synthesis can facilitate enhanced production of particular amino acids and related metabolites. In one embodiment, expression of a dihydrodipicolinate synthase polypeptide is deficient in the bacterium (e.g., an endogenous dapA gene in the bacterium is mutated or deleted). In various embodiments, expression of one or more of the following polypeptides is reduced: an mcbR gene product, homoserine dehydrogenase, homoserine kinase, methionine adenosyltransferase, homoserine O-acetyltransferase, phosphoenolpyruvate carboxykinase, diaminopimelate dehydrogenase polypeptide, an ABC transport system ATP-binding protein polypeptide, an ABC transport system permease protein polypeptide, a glucose-6-phosphate isomerase polypeptide, an NCgl2640 polypeptide, and an ABC transport system substrate-binding protein polypeptide. In certain embodiments the expression or activity of adenosyl transferase (pduO) is reduced or eliminated.

Various bacteria are described, including a host bacterium (e.g., a coryneform bacterium or a bacterium of the family Enterobacteriaceae such as an Escherichia coli bacterium) comprising at least one (e.g., one, two, three, four or more) recombinant nucleic acid molecule(s) selected from: (a) a nucleic acid molecule comprising a sequence encoding a bacterial aspartokinase polypeptide or a functional variant thereof; (b) a nucleic acid molecule comprising a sequence encoding a bacterial aspartate semialdehyde dehydrogenase polypeptide or a functional variant thereof; (c) a nucleic acid molecule comprising a sequence encoding a bacterial phosphoenolpyruvate carboxylase polypeptide or a functional variant thereof; (d) a nucleic acid molecule comprising a sequence encoding a bacterial pyruvate carboxylase polypeptide or a functional variant thereof; (e) a nucleic acid molecule comprising a sequence encoding a bacterial dihydrodipicolinate synthase polypeptide or a functional variant thereof; (f) a nucleic acid molecule comprising a sequence encoding a bacterial homoserine dehydrogenase polypeptide or a functional variant thereof; (g) a nucleic acid molecule comprising a sequence encoding a bacterial homoserine O-acetyltransferase polypeptide or a functional variant thereof; (h) a nucleic acid molecule comprising a sequence encoding a bacterial O-acetylhomoserine sulfhlydrylase polypeptide or a functional variant thereof; (i) a nucleic acid molecule comprising a sequence encoding a bacterial methionine adenosyltransferase polypeptide or a functional variant thereof; (j) a nucleic acid molecule comprising a sequence encoding a bacterial mcbR gene product polypeptide or a functional variant thereof; (k) a nucleic acid molecule comprising a sequence encoding a bacterial O-succinylhomoserine/acetylhomoserine (thiol)-lyase polypeptide or a functional variant thereof; (l) a nucleic acid molecule comprising a sequence encoding a bacterial cystathionine beta-lyase polypeptide or a functional variant thereof; (m) a nucleic acid molecule comprising a sequence encoding a bacterial 5-methyltetrahydrofolate homocysteine methyltransferase polypeptide or a functional variant thereof; (n) a nucleic acid molecule comprising a sequence encoding a bacterial 5-methyltetrahydropteroyltriglutamate-homocysteine methyltransferase polypeptide or a functional variant thereof; (o) a nucleic acid molecule comprising a sequence encoding a bacterial phosphoenolpyruvate carboxykinase polypeptide or a functional variant thereof; (p) a nucleic acid molecule comprising a sequence encoding a bacterial diaminopimelate dehydrogenase polypeptide or a functional variant thereof or (q) a nucleic acid molecule encoding a polypeptide listed in Table 6.

In various embodiments, the nucleic acid molecule is an isolated nucleic acid molecule (e.g., the nucleic acid molecule is free of nucleotide sequences that naturally flank the sequence in the organism from which the nucleic acid molecule is derived, e.g., the nucleic acid molecule is a recombinant nucleic acid molecule). A recombinant nucleic acid molecule is a nucleic acid molecule that is either not naturally-occurring or is inserted into a nucleic acid molecule such that it is flanked by sequences that do not flank the nucleic acid molecule in the organism from which it is derived. For example, a nucleic acid molecule encoding E. coli beta-galactosidase that is inserted into an expression vector is a recombinant nucleic acid molecule as is a nucleic acid molecule encoding E. coli beta-galactosidase that is inserted into the E. coli genome at a location other than its native location. Another example of a recombinant nucleic acid molecule is a nucleic acid molecule encoding E. coli beta-galactosidase that is inserted into a genome other than the E. coli genome. Any of the nucleic acid molecules herein can be a recombinant nucleic acid molecule unless otherwise specified.

The encoded polypeptide, i.e., the polypeptide in any of Tables 1-6, can be homologous to or heterologous to the host cell. Thus, the polypeptide can have the sequence of a polypeptide that is normally found in cells of the host cell species (homologous) or the polypeptide can have the sequence of a polypeptide that naturally occurs in cells of a species other than the host species. Thus, Mycobacterium smegmatis aspartokinase polypeptide is homologous to the host cell when expressed in Mycobacterium smegmatis and is heterologous to the host cell when expressed in Amycolatopsis mediterranei.

In various embodiments, the polypeptide is selected from an Enterobacteriaceae polypeptide, an Actinomycetes polypeptide, or a variant thereof. In various embodiments, the polypeptide is a polypeptide of one of the following Actinomycetes species: Mycobacterium smegmatis, Nocardia farcinica, Streptomyces coelicolor, Thermobifida fusca, Amycolatopsis mediterranei and coryneform bacteria, including Corynebacterium glutamicum and Corynebacterium diphtheriae. In various embodiments, the polypeptide is a polypeptide of one of the following Enterobacteriaceae species: Erwinia chysanthemi, Erwinia Carotovora, and Escherichia coli. In various embodiments, the polypeptide is a polypeptide of one of the following: Bacillus halodurans, Clostridium acetobutylicum, and Lactobacillus plantarum. In various embodiments the polypeptide is a polypeptide of one of the following: Mycobacterium smegmatis, Thermobifida fusca, and Streptomyces coelicolor.

In various embodiments, the polypeptide is a variant polypeptide with reduced feedback inhibition (e.g., relative to a wild-type form of the polypeptide). In various embodiments, the bacterium further comprises additional heterologous bacterial gene products or recombinant homologous bacterial gene products involved in amino acid production. In various embodiments, the bacterium further comprises a nucleic acid molecule encoding a heterologous bacterial polypeptide described herein or a recombinant nucleic acid molecule encoding a homologous bacterial polypeptide described herein (e.g., a nucleic acid molecule encoding a heterologous bacterial homoserine dehydrogenase polypeptide). In various embodiments, the bacterium further comprises a nucleic acid molecule encoding a homologous bacterial polypeptide (i.e., a bacterial polypeptide that is native to the host species or a functional variant thereof), such as a bacterial polypeptide described herein. The homologous bacterial polypeptide can be expressed at high levels and/or conditionally expressed. For example, the nucleic acid encoding the homologous bacterial polypeptide can be operably linked to a promoter that allows expression of the polypeptide at a level that is higher than the wild-type level, the nucleic acid can express the protein at a wild-type level, but increase overall expression by increasing the number of copies of nucleic acid encoding the homologous polypeptide in the cell and/or the nucleic acid may be present in multiple copies in the bacterium. In various embodiments, the nucleic acid molecule encoding the heterologous or homologous bacterial polypeptide is present on an episome within the host organism. In various embodiments, the nucleic acid molecule encoding the heterologous or homologous bacterial polypeptide is integrated into the genome of the host organism. In some embodiments, the host organism harbors both one or more episomal nucleic acid molecules that encode a specified homologous or heterologous bacterial polypeptide and one or more molecules that encode a specified homologous or heterologous bacterial polypeptide that are integrated into the genome of the host organism.

In various embodiments the bacterial aspartokinase or functional variant thereof is chosen from: (a) a Mycobacterium smegmatis aspartokinase polypeptide or a functional variant thereof, (b) an Amycolatopsis mediterranei aspartokinase polypeptide or a functional variant thereof, (c) a Streptomyces coelicolor aspartokinase polypeptide or a functional variant thereof, (d) a Thermobifida fusca aspartokinase polypeptide or a functional variant thereof, (e) an Erwinia chrysanthemi aspartokinase polypeptide or a functional variant thereof, and (f) a Shewanella oneidensis aspartokinase polypeptide or a functional variant thereof. In certain embodiments, the heterologous bacterial aspartokinase polypeptide is an Escherichia coli aspartokinase polypeptide or a functional variant thereof. In certain embodiments, the heterologous bacterial aspartokinase polypeptide is a Corynebacterium glutamicum aspartokinase polypeptide or a functional variant thereof. In certain embodiments the heterologous bacterial asparatokinase polypeptide or functional variant thereof has reduced feedback inhibition.

In various embodiments the bacterial aspartate semialdehyde dehydrogenase polypeptide or functional variant thereof is chosen from: (a) a Mycobacterium smegmatis aspartate semialdehyde dehydrogenase polypeptide or a functional variant thereof, (b) an Amycolatopsis mediterranei aspartate semialdehyde dehydrogenase polypeptide or a functional variant thereof, (c) a Streptomyces coelicolor aspartate semialdehyde dehydrogenase polypeptide or a functional variant thereof, and (d) a Thermobifida fusca aspartate semialdehyde dehydrogenase polypeptide or a functional variant thereof. In certain embodiments, the bacterial aspartate semialdehyde dehydrogenase polypeptide is an Escherichia coli aspartate semialdehyde dehydrogenase polypeptide or a functional variant thereof. In certain embodiments, the bacterial aspartate semialdehyde dehydrogenase polypeptide is a Corynebacterium glutamicum aspartate semialdehyde dehydrogenase polypeptide or a functional variant thereof.

In various embodiments the bacterial phosphoenolpyruvate carboxylase polypeptide or functional variant thereof is chosen from: (a) a Mycobacterium smegmatis phosphoenolpyruvate carboxylase polypeptide or a functional variant thereof, (b) a Streptomyces coelicolor phosphoenolpyruvate carboxylase polypeptide or a functional variant thereof, (c) a Thermobifida fusca phosphoenolpyruvate carboxylase polypeptide or a functional variant thereof, and (d) an Erwinia chrysanthemi phosphoenolpyruvate carboxylase polypeptide or a functional variant thereof. In certain embodiments, the bacterial phosphoenolpyruvate carboxylase polypeptide is an Escherichia coli phosphoenolpyruvate carboxylase polypeptide or a functional variant thereof. In certain embodiments, the heterologous bacterial phosphoenolpyruvate carboxylase polypeptide is a Corynebacterium glutamicum phosphoenolpyruvate carboxylase polypeptide or a functional variant thereof.

In various embodiments the bacterial pyruvate carboxylase polypeptide or functional variant thereof is chosen from: (a) a Mycobacterium smegmatis pyruvate carboxylase polypeptide or a functional variant thereof, (b) a Streptomyces coelicolor pyruvate carboxylase polypeptide or a functional variant thereof, and (c) a Thermobifida fusca pyruvate carboxylase polypeptide or a functional variant thereof. In certain embodiments, the bacterial pyruvate carboxylase polypeptide is a Corynebacterium glutamicum pyruvate carboxylase or a functional variant thereof.

In various embodiments the host bacterium is chosen from a coryneform bacterium or a bacterium of the family Enterobacteriaceae such as an Escherichia coli bacterium. Coryneform bacteria include, without limitation, Corynebacterium glutamicum, Corynebacterium acetoglutamicum, Corynebacterium melassecola, Corynebacterium thermoaminogenes, Brevibacterium lactofermentum, Brevibacterium lactis, and Brevibacterium flavum.

In various embodiments, the Mycobacterium smegmatis aspartokinase polypeptide comprises at least one amino acid change chosen from: an alanine changed to a Group 1 amino acid residue at position 279; a serine changed to a Group 6 amino acid residue at position 301; a threonine changed to a Group 2 amino acid residue at position 311; and a glycine changed to a Group 3 amino acid residue at position 345; the Mycobacterium smegmatis aspartokinase comprises at least one amino acid change chosen from: an alanine changed to a proline at position 279, a serine changed to a tyrosine at position 301, a threonine changed to an isoleucine at position 311, and a glycine changed to an aspartate at position 345.

In various embodiments, the Amycolatopsis mediterranei aspartokinase polypeptide comprises at least one amino acid change chosen from: an alanine changed to a Group 1 amino acid residue at position 279; a serine changed to a Group 6 amino acid residue at position 301; a threonine changed to a Group 2 amino acid residue at position 311; and a glycine changed to a Group 3 amino acid residue at position 345.

In various embodiments the Amycolatopsis mediterranei aspartokinase polypeptide comprises at least one amino acid change chosen from: an alanine changed to a proline at position 279; a serine changed to a tyrosine at position 301; a threonine changed to an isoleucine at position 311; and a glycine changed to an aspartate at position 345.

In various embodiments the Streptomyces coelicolor aspartokinase polypeptide comprises at least one amino acid change chosen from: an alanine changed to a Group 1 amino acid residue at position 282; a serine changed to a Group 6 amino acid residue at position 304; a serine changed to a Group 2 amino acid residue at position 314; and a glycine changed to a Group 3 amino acid residue at position 348.

In various embodiments the Streptomyces coelicolor aspartokinase polypeptide comprises at least one amino acid change chosen from: an alanine changed to a proline at position 282; a serine changed to a tyrosine at position 304; a serine changed to an isoleucine at position 314; and a glycine changed to an aspartate at position 348.

In various embodiments the Erwinia chrysanthemi aspartokinase polypeptide comprises at least one amino acid change chosen from: a glycine changed to a Group 3 amino acid residue at position 328; a leucine changed to a Group 6 amino acid residue at position 330; a serine changed to a Group 2 amino acid residue at position 350; and a valine changed to a Group 2 amino acid residue other than valine at position 352.

In various embodiments the Erwinia chrysanthemi aspartokinase polypeptide comprises at least one amino acid change chosen from: a glycine changed to an aspartate at position 328; a leucine changed to a phenylalanine at position 330; a serine changed to an isoleucine at position 350; and a valine changed to a methionine at position 352.

In various embodiments the Shewanella oneidensis aspartokinase polypeptide comprises at least one amino acid change chosen from: a glycine changed to a Group 3 amino acid residue at position 323; a leucine changed to a Group 6 amino acid residue at position 325; a serine changed to a Group 2 amino acid residue at position 345; and a valine changed to a Group 2 amino acid residue other than valine at position 347.

In various embodiments the Shewanella oneidensis aspartokinase polypeptide comprises at least one amino acid change chosen from: a glycine changed to an aspartate at position 323; a leucine changed to a phenylalanine at position 325; a serine changed to an isoleucine at position 345; and a valine changed to a methionine at position 347.

In various embodiments the Corynebacterium glutamicum aspartokinase polypeptide comprises at least one amino acid change chosen from: an alanine changed to a Group 1 amino acid other than alanine at position 279; a serine changed to a Group 6 amino acid residue at position 301; a threonine changed to a Group 2 amino acid residue at position 311; and a glycine changed to a Group 3 amino acid residue at position 345.

In various embodiments the Corynebacterium glutamicum aspartokinase polypeptide comprises at least one amino acid change chosen from: an alanine changed to a proline at position 279; a serine changed to a tyrosine at position 301; a threonine changed to an isoleucine at position 311; and a glycine changed to an aspartate at position 345.

In various embodiments the Escherichia coli aspartokinase polypeptide comprises at least one amino acid change chosen from: a glycine changed to a Group 3 amino acid residue at position 323; a leucine changed to a Group 6 amino acid residue at position 325; a serine changed to a Group 2 amino acid residue at position 345; and a valine changed to a Group 2 amino acid residue other than valine at position 347.

In various embodiments the Escherichia coli aspartokinase polypeptide comprises at least one amino acid change chosen from: a glycine changed to an aspartate at position 323; a leucine changed to a phenylalanine at position 325; a serine changed to an isoleucine at position 345; and a valine changed to a methionine at position 347.

In various embodiments, the Corynebacterium glutamicum pyruvate carboxylase polypeptide or variant thereof comprises a proline changed to Group 4 amino acid residue at position 458. In various embodiments, the Corynebacterium glutamicum pyruvate carboxylase polypeptide or variant thereof comprises a proline changed to a serine at position 458.

In various embodiments, the Mycobacterium smegmatis pyruvate carboxylase polypeptide or variant thereof comprises a proline changed to Group 4 amino acid residue at position 448. In various embodiments, the Mycobacterium smegmatis pyruvate carboxylase polypeptide or variant thereof comprises a proline changed to a serine at position 448.

In various embodiments, the Streptomyces coelicolor pyruvate carboxylase polypeptide or variant thereof comprises a proline changed to Group 4 amino acid residue at position 449. In various embodiments, the Streptomyces coelicolor pyruvate carboxylase polypeptide or variant thereof comprises a proline changed to a serine at position 449.

Also featured is a coryneform bacterium or a bacterium of the family Enterobacteriaceae such as an Escherichia coli bacterium comprising a nucleic acid molecule that encodes a bacterial dihydrodipicolinate synthase or a functional variant thereof.

In various embodiments the bacterial dihydrodipicolinate synthase polypeptide or functional variant thereof is chosen from: a Mycobacterium smegmatis dihydrodipicolinate synthase polypeptide or a functional variant thereof; a Streptomyces coelicolor dihydrodipicolinate synthase polypeptide or a functional variant thereof; a Thermobifida fusca dihydrodipicolinate synthase polypeptide or a functional variant thereof; and an Erwinia chrysanthemi dihydrodipicolinate synthase polypeptide or a functional variant thereof. In certain embodiments, the heterologous bacterial dihydrodipicolinate synthase polypeptide or functional variant thereof with reduced feedback inhibition is an Escherichia coli dihydrodipicolinate synthase polypeptide or a functional variant thereof. In certain embodiments the heterologous bacterial dihydrodipicolinate synthase polypeptide or functional variant thereof has reduced feedback inhibition.

In various embodiments the Erwinia chrysanthemi dihydrodipicolinate synthase polypeptide comprises at least one amino acid change chosen from: an asparagine changed to a Group 2 amino acid residue at position 80; a leucine changed to a Group 6 amino acid residue at position 88; and a histidine changed to a Group 6 amino acid residue at position 118.

In various embodiments the Erwinia chrysanthemi dihydrodipicolinate synthase polypeptide comprises at least one amino acid change chosen from: an asparagine changed to an isoleucine at position 80; a leucine changed to a phenylalanine at position 88; and a histidine changed to a tyrosine at position 118.

In various embodiments, the Streptomyces coelicolor dihydrodipicolinate synthase polypeptide comprises at least one amino acid change chosen from: an asparagine changed to a Group 2 amino acid residue at position 89; a leucine changed to a Group 6 amino acid residue at position 97; and a histidine changed to a Group 6 amino acid residue at position 127.

In various embodiments the Streptomyces coelicolor dihydrodipicolinate synthase polypeptide comprises at least one amino acid change chosen from: an asparagine changed to an isoleucine at position 89; a leucine changed to a phenylalanine at position 97; and a histidine changed to a tyrosine at position 127.

In various embodiments the Escherichia coli dihydrodipicolinate synthase polypeptide comprises at least one amino acid change chosen from: an asparagine changed to a Group 2 amino acid residue at position 80; an alanine changed to a Group 2 amino acid residue at position 81; a glutamatate changed to a Group 5 amino acid residue at position 84; a leucine changed to a Group 6 amino acid residue at position 88; and a histidine changed to a Group 6 amino acid at position 118.

In various embodiments the Escherichia coli dihydrodipicolinate synthase polypeptide comprises at least one amino acid change chosen from: an asparagine changed to an isoleucine at position 80; an alanine changed to a valine at position 81; a glutamate changed to a lysine at position 84; a leucine changed to a phenylalanine at position 88; and a histidine changed to a tyrosine at position 118.

In various embodiments the bacterial homoserine dehydrogenase polypeptide is chosen from: (a) a Mycobacterium smegmatis homoserine dehydrogenase polypeptide or functional variant thereof; (b) a Streptomyces coelicolor homoserine dehydrogenase polypeptide or a functional variant thereof; (c) a Thermobifida fusca homoserine dehydrogenase polypeptide or a functional variant thereof; and (d) an Erwinia chrysanthemi homoserine dehydrogenase polypeptide or a functional variant thereof. In certain embodiments, the bacterial homoserine dehydrogenase polypeptide is a homoserine dehydrogenase polypeptide from a coryneform bacteria or a functional variant thereof (e.g., a Corynebacterium glutamicum homoserine dehydrogenase polypeptide or functional variant thereof, or a Brevibacterium lactofermentum homoserine dehydrogenase polypeptide or functional variant thereof). In certain embodiments, the homoserine dehydrogenase polypeptide or functional variant thereof is an Escherichia coli homoserine dehydrogenase polypeptide or a functional variant thereof. In certain embodiments the heterologous homoserine dehydrogenase polypeptide or functional variant thereof has reduced feedback inhibition.

In various embodiments the Corynebacterium glutamicum or Brevibacterium lactofermentum homoserine dehydrogenase polypeptide comprises at least one amino acid change chosen from: a leucine change to a Group 6 amino acid residue at position 23; a valine changed to a Group 1 amino acid residue at position 59; a valine changed to another Group 2 amino acid residue at position 104; a glycine changed to Group 3 amino acid residue at position 378; and an alteration that truncates the homoserine dehydrogenase protein after the lysine amino acid residue at position 428. In one embodiment, the Corynebacterium glutamicum or Brevibacterium lactofermentum homoserine dehydrogenase polypeptide is encoded by the hom^{dr} sequence described in WO93/09225 (SEQ ID NO. 3).

In various embodiments the Corynebacterium glutamicum or Brevibacterium lactofermentum homoserine dehydrogenase polypeptide comprises at least one amino acid change chosen from: a leucine changed to a phenylalanine at position 23; valine changed to an alanine at position 59; a valine changed to an isoleucine at position 104; and a glycine changed to a glutamic acid at position 378.

In various embodiments the Mycobacterium smegmatis homoserine dehydrogenase polypeptide comprises at least one amino acid change chosen from: a valine change to a Group 6 amino acid residue at position 10; a valine changed to a Group 1 amino acid residue at position 46; and a glycine changed to Group 3 amino acid residue at position 364.

In various embodiments the Mycobacterium smegmatis homoserine dehydrogenase polypeptide comprises at least one amino acid change chosen from: a valine changed to a phenylalanine at position 10; valine changed to an alanine at position 46; and a glycine changed to a glutamic acid at position 378.

In various embodiments the Streptomyces coelicolor homoserine dehydrogenase polypeptide comprises at least one amino acid change chosen from: a leucine change to a Group 6 amino acid residue at position 10; a valine changed to a Group 1 amino acid residue at position 46; a glycine changed to Group 3 amino acid residue at position 362; an alteration that truncates the homoserine dehydrogenase protein after the arginine amino acid residue at position 412. In various embodiments the Streptomyces coelicolor homoserine dehydrogenase polypeptide comprises at least one amino acid change chosen from: a leucine changed to a phenylalanine at position 10; a valine changed to an alanine at position 46; and a glycine changed to a glutamic acid at position 362.

In various embodiments the Thermobifida fusca homoserine dehydrogenase polypeptide comprises at least one amino acid change chosen from: a leucine change to a Group 6 amino acid residue at position 192; a valine changed to a Group 1 amino acid residue at position 228; a glycine changed to Group 3 amino acid residue at position 545. In various embodiments, the Thermobifida fusca homoserine dehydrogenase polypeptide is truncated after the arginine amino acid residue at position 595.

In various embodiments the Thermobifida fusca homoserine dehydrogenase polypeptide comprises at least one amino acid change chosen from: a leucine changed to a phenylalanine at position 192; valine changed to an alanine at position 228; and a glycine changed to a glutamic acid at position 545.

In various embodiments the Escherichia coli homoserine dehydrogenase polypeptide comprises at least one amino acid change chosen from: a glycine changed to a Group 3 amino acid residue at position 330; and a serine changed to a Group 6 amino acid residue at position 352. In various embodiments the Escherichia coli homoserine dehydrogenase polypeptide comprises at least one amino acid change chosen from: a glycine changed to an aspartate at position 330; and a serine changed to a phenylalanine at position 352.

In various embodiments the bacterial O-homoserine acetyltransferase polypeptide is chosen from: a Mycobacterium smegmatis O-homoserine acetyltransferase polypeptide or functional variant thereof; a Streptomyces coelicolor O-homoserine acetyltransferase polypeptide or a functional variant thereof; a Thermobifida fusca O-homoserine acetyltransferase polypeptide or a functional variant thereof; and an Erwinia chrysanthemi O-homoserine acetyltransferase polypeptide or a functional variant thereof. In certain embodiments, the bacterial O-homoserine acetyltransferase polypeptide is an O-homoserine acetyltransferase polypeptide from Corynebacterium glutamicum or a functional variant thereof. In certain embodiments the heterologous O-homoserine acetyltransferase polypeptide or functional variant thereof has reduced feedback inhibition.

In various embodiments the bacterial O-acetylhomoserine sulfhydrylase polypeptide is chosen from: (a) a Mycobacterium smegmatis O-acetylhomoserine sulfhydrylase polypeptide or functional variant thereof; (b) a Streptomyces coelicolor O-acetylhomoserine sulfhydrylase polypeptide or a functional variant thereof; and (c) a Thermobifida fusca O-acetylhomoserine sulfhydrylase polypeptide or a functional variant thereof. In certain embodiments, the bacterial O-acetylhomoserine sulfhydrylase polypeptide is an O-acetylhomoserine sulfhydrylase polypeptide from Corynebacterium glutamicum or a functional variant thereof. In certain embodiments the heterologous O-acetylhomoserine sulfhydrylase polypeptide or functional variant thereof has reduced feedback inhibition.

In various embodiments the bacterial methionine adenosyltransferase polypeptide is chosen from: a Mycobacterium smegmatis methionine adenosyltransferase polypeptide or functional variant thereof; a Streptomyces coelicolor methionine adenosyltransferase polypeptide or a functional variant thereof; a Thermobifida fusca methionine adenosyltransferase polypeptide or a functional variant thereof; and an Erwinia chrysanthemi methionine adenosyltransferase polypeptide or a functional variant thereof. In certain embodiments, the bacterial methionine adenosyltransferase polypeptide is a methionine adenosyltransferase polypeptide from Corynebacterium glutamicum or a functional variant thereof. In certain embodiments, the bacterial methionine adenosyltransferase polypeptide is a methionine adenosyltransferase polypeptide from Escherichia coli or a functional variant thereof. In certain embodiments the heterologous methionine adenosyltransferase polypeptide or functional variant thereof has reduced feedback inhibition.

In various embodiments the Mycobacterium smegmatis methionine adenosyltransferase polypeptide comprises a valine change to a Group 3 amino acid residue at position 196. In various embodiments the Mycobacterium smegmatis methionine adenosyltransferase polypeptide comprises a valine change to a glutamic acid residue at position 196.

In various embodiments the Streptomyces coelicolor methionine adenosyltransferase polypeptide comprises a valine change to a Group 3 amino acid residue at position 195. In various embodiments the Streptomyces coelicolor methionine adenosyltransferase polypeptide comprises a valine change to a glutamic acid residue at position 195. In various embodiments the Thermobifida fusca methionine adenosyltransferase polypeptide comprises a valine change to a Group 3 amino acid residue at position 195. In various embodiments the Thermobifida fusca methionine adenosyltransferase polypeptide comprises a valine change to a glutamic acid residue at position 195.

In various embodiments the Erwinia chrysanthemi methionine adenosyltransferase polypeptide comprises a valine change to a Group 3 amino acid residue at position 185. In various embodiments the Erwinia chrysanthemi methionine adenosyltransferase polypeptide comprises a valine change to a glutamic acid residue at position 185.

In various embodiments the Corynebacterium glutamicum methionine adenosyltransferase polypeptide comprises a valine change to a Group 3 amino acid residue at position 200. In various embodiments the Corynebacterium glutamicum methionine adenosyltransferase polypeptide comprises a valine change to a glutamic acid residue at position 200.

In various embodiments the Escherichia coli methionine adenosyltransferase polypeptide comprises a valine change to a Group 3 amino acid residue at position 185. In various embodiments the Escherichia coli methionine adenosyltransferase polypeptide comprises a valine change to a glutamic acid residue at position 185.

A host cell having reduced activity or expression of MetK and/or DapA can be useful for producing methionine. Thus, the host cell can have at least one mutation (e.g., insertion, deletion or missense mutation) in the sequences encoding MetK, the sequence encoding DapA or both. Expression of these genes can be decreased by mutation or deletion of expression control sequences.

In various embodiments the bacterium further comprises at least one of: (a) a nucleic acid molecule (e.g., a recombinant nucleic acid molecule) encoding a bacterial homoserine dehydrogenase polypeptide or a functional variant thereof; (b) a nucleic acid molecule (e.g., a recombinant nucleic acid molecule) encoding a bacterial O-homoserine acetyltransferase polypeptide or a functional variant thereof; (c) a nucleic acid molecule (e.g., a recombinant nucleic acid molecule) encoding a bacterial O-acetylhomoserine sulfhydrylase polypeptide or a functional variant thereof. In certain embodiments one or more of the polypeptides or functional variants thereof has reduced feedback inhibition.

In various embodiments the heterologous bacterial homoserine dehydrogenase polypeptide is chosen from: a Mycobacterium smegmatis homoserine dehydrogenase polypeptide or functional variant thereof; a Streptomyces coelicolor homoserine dehydrogenase polypeptide or a functional variant thereof; a Thermobifida fusca homoserine dehydrogenase polypeptide or a functional variant thereof; an Escherichia coli homoserine dehydrogenase polypeptide or a functional variant thereof; a Corynebacterium glutamicum homoserine dehydrogenase polypeptide or a functional variant thereof; and an Erwinia chrysanthemi homoserine dehydrogenase polypeptide or a functional variant thereof. In certain embodiments the heterologous homoserine dehydrogenase polypeptide or functional variant thereof has reduced feedback inhibition.

In various embodiments the heterologous bacterial O-homoserine acetyltransferase polypeptide is chosen from: a Mycobacterium smegmatis O-homoserine acetyltransferase polypeptide or functional variant thereof; a Streptomyces coelicolor O-homoserine acetyltransferase polypeptide or a functional variant thereof; a Thermobifida fusca O-homoserine acetyltransferase polypeptide or a functional variant thereof; an Erwinia chrysanthemi O-homoserine acetyltransferase polypeptide or a functional variant thereof; an Escherichia coli O-homoserine acetyltransferase polypeptide or a functional variant thereof; and a Corynebacterium glutamicum O-homoserine acetyltransferase polypeptide or a functional variant thereof. In certain embodiments the heterologous O-homoserine acetyltransferase polypeptide or functional variant thereof has reduced feedback inhibition.

In various embodiments the heterologous bacterial O-acetylhomoserine sulfhydrylase polypeptide is chosen from: a Mycobacterium smegmatis O-acetylhomoserine sulfhydrylase or functional variant thereof; a Streptomyces coelicolor O-acetylhomoserine sulfhydrylase polypeptide or a functional variant thereof; a Thermobifida fusca O-acetylhomoserine sulfhydrylase polypeptide or a functional variant thereof; and a Corynebacterium glutamicum O-acetylhomoserine sulfhydrylase polypeptide or a functional variant thereof. In certain embodiments the heterologous O-acetylhomoserine sulfhydrylase polypeptide or functional variant thereof has reduced feedback inhibition.

In various embodiments the bacterium further comprises a nucleic acid molecule (e.g., a recombinant nucleic acid molecule) encoding a bacterial methionine adenosyltransferase polypeptide (e.g., a Mycobacterium smegmatis methionine adenosyltransferase polypeptide or functional variant thereof; a Streptomyces coelicolor methionine adenosyltransferase polypeptide or a functional variant thereof; a Thermobifida fusca methionine adenosyltransferase polypeptide or a functional variant thereof; an Erwinia chrysanthemi methionine adenosyltransferase polypeptide or a functional variant thereof; an Escherichia coli methionine adenosyltransferase polypeptide or a functional variant thereof; or a Corynebacterium glutamicum methionine adenosyltransferase polypeptide or a functional variant thereof).

In various embodiments the bacterium further comprises a nucleic acid molecule (e.g., a recombinant nucleic acid molecule) encoding a cobalamin-dependent methionine synthesis polypeptide (MetH) (e.g., a Mycobacterium smegmatis cobalamin-dependent methionine synthesis polypeptide or a functional variant thereof; a Streptomyces coelicolor cobalamin-dependent methionine synthesis polypeptide or a functional variant thereof; a Thermobifida fusca cobalamin-dependent methionine synthesis polypeptide or a functional variant thereof; an Erwinia chrysanthemi cobalamin-dependent methionine synthesis polypeptide or a functional variant thereof; an Escherichia coli cobalamin-dependent methionine synthesis polypeptide or a functional variant thereof; or a Corynebacterium glutamicum cobalamin-dependent methionine synthesis polypeptide or a functional variant thereof).

In various embodiments the bacterium further comprises a nucleic acid molecule (e.g., a recombinant nucleic acid molecule) encoding a cobalamin-independent methionine synthesis polypeptide (MetE) (e.g., a Mycobacterium smegmatis cobalamin-independent methionine synthesis polypeptide or a functional variant thereof; a Streptomyces coelicolor cobalamin-independent methionine synthesis polypeptide or a functional variant thereof; a Thermobifida fusca cobalamin-independent methionine synthesis polypeptide or a functional variant thereof; an Erwinia chrysanthemi cobalamin-independent methionine synthesis polypeptide or a functional variant thereof; an Escherichia coli cobalamin-independent methionine synthesis polypeptide or a functional variant thereof; or a Corynebacterium glutamicum cobalamin-independent methionine synthesis polypeptide or a functional variant thereof).

“Aspartic acid family of amino acids and related metabolites” encompasses, e.g., L-aspartate, β-aspartyl phosphate, L-aspartate-β-semialdehyde, L-2,3-dihydrodipicolinate, L-Δ¹-piperideine-2,6-dicarboxylate, N-succinyl-2-amino-6-keto-L-pimelate, N-succinyl-2, 6-L, L-diaminopimelate, L, L-diaminopimelate, D, L-diaminopimelate, L-lysine, homoserine, O-acetyl-L-homoserine, O-succinyl-L-homoserine, cystathionine, L-homocysteine, L-methionine, S-adenosyl-L-methionine (S-adenosylmethionine), O-phospho-L-homoserine, threonine, 2-oxobutanoate, (S)-2-aceto-2-hydroxybutanoate, (S)-2-hydroxy-3-methyl-3-oxopentanoate, (R)-2,3-Dihydroxy-3-methylpentanoate, (R)-2-oxo-3-methylpentanoate, L-isoleucine, and L-asparagine as well as other conformational isomers of these compounds. In various embodiments the aspartic acid family of amino acids and related metabolites encompasses aspartic acid, asparagine, lysine, threonine, methionine, isoleucine, and S-adenosylmethionine.

A polypeptide or functional variant thereof with “reduced feedback inhibition” includes a polypeptide that is less inhibited by the presence of an inhibitory factor as compared to a wild-type form of the polypeptide or a polypeptide that is less inhibited by the presence of an inhibitory factor as compared to the corresponding endogenous polypeptide expressed in the organism into which the variant has been introduced. For example, a wild-type aspartokinase from E. coli or C. glutamicum may have 10-fold less activity in the presence of a given concentration of lysine, or lysine plus threonine, respectively. A variant with reduced feedback inhibition may have, for example, 5-fold less, 2-fold less, or wild-type levels of activity in the presence of the same concentration of lysine.

Heterologous proteins may be encoded by genes of any bacterial organism other than the host bacterial species. The heterologous genes can be genes from the following, non-limiting list of bacteria: Mycobacterium smegmatis; Amycolatopsis mediterranei; Streptomyces coelicolor; Thermobifida fusca; Erwinia chrysanthemi; Erwinia carotovora; Streptomyces coelicolor; Shewanella oneidensis; Lactobacillus plantarum; Bifidobacterium longum; Bacillus sphaericus; and Pectobacterium chrysanthemi; Clostridium acetobutylicum; Bacillus halodurans; Escherichia coli; Corynebacterium diptheriae; and Nocardia farcinica.

Of course, heterologous genes for host strains from the Enterobacteriaceae family also include genes from coryneform bacteria. Likewise, heterologous genes for host strains of coryneform bacteria also include genes from Enterobacteriaceae family members. In certain embodiments, the host strain is Escherichia coli and the heterologous gene is a gene of a species other than a coryneform bacteria. In certain embodiments, the host strain is a coryneform bacteria and the heterologous gene is a gene of a species other than Escherichia coli. In certain embodiments, the host strain is Escherichia coli and the heterologous gene is a gene of a species other than Corynebacterium glutamicum. In certain embodiments, the host strain is Corynebacterium glutamicum and the heterologous gene is a gene of a species other than Escherichia coli. In various embodiments, the polypeptide is encoded by a gene obtained from an organism of the order Actinomycetales. In various embodiments, the nucleic acid molecule is obtained from Mycobacterium smegmatis, Streptomyces coelicolor, Thermobifida fusca, Amycolatopsis mediterranei, Nocardia farcinica or a coryneform bacteria such as Corynebacterium glutamicum or Corynebacterium diptheriae. In various embodiments, the nucleic acid molecule is obtained from Mycobacterium smegmatis, Streptomyces coelicolor, or Thermobifida fusca. In various embodiments, the protein is encoded by a gene obtained from an organism of the family Enterobacteriaceae. In various embodiments, the nucleic acid molecule is obtained from Erwinia chysanthemi, Erwinia Carotovora, or Escherichia coli.

In various embodiments, the host bacterium (e.g., coryneform bacterium or bacterium of the family Enterobacteriaceae) in addition to harboring a nucleic acid molecule encoding a heterologous polypeptide also has increased levels of a polypeptide encoded by a gene from the host bacterium (e.g., from a coryneform bacterium or a bacterium of the family Enterobacteriaceae such as an Escherichia coli bacterium). In various embodiments, increased levels of a polypeptide encoded by a gene from the host bacterium may result from one or more of the following: introduction of additional copies of a gene from the host bacterium regulated by the naturally associated promoter; introduction of additional copies of a gene from the host bacterium under the control of a promoter, e.g., a promoter more optimal for amino acid production than the naturally occurring promoter, either from the host, a heterologous organism, or a non-naturally occurring nucleic acid sequence; or the replacement of the naturally occurring promoter of the gene from the host bacterium with a promoter more optimal for amino acid production, either from the host, a heterologous organism, or a non-naturally occurring nucleic acid sequence. Nucleic acid molecules that include sequences encoding a homologous or heterologous polypeptide (e.g., vectors that encode one or more polypeptides) may be integrated into the host genome or exist as an episomal plasmid.

In various embodiments, the host bacterium has reduced expression or activity of a polypeptide. Reducing the expression or activity of particular polypeptides involved in amino acid synthesis can facilitate enhanced production of particular amino acids and related metabolites. Reduced expression or activity can arise from alterations in the coding sequence or a regulatory sequence. In one embodiment, expression of a dihydrodipicolinate synthase polypeptide is reduced in the bacterium (e.g., an endogenous dapA gene in the bacterium is mutated or deleted). In various embodiments, expression of one or more of the following polypeptides is deficient: an mcbR gene product, homoserine dehydrogenase, homoserine kinase, methionine adenosyltransferase, homoserine O-acetyltransferase, phosphoenolpyruvate carboxykinase, an adenosyl transferase polypeptide, a diaminopimelate dehydrogenase polypeptide, an ABC transport system ATP-binding protein polypeptide, an ABC transport system permease protein polypeptide, a glucose-6-phosphate isomerase polypeptide, an NCgl2640 polypeptide, and an ABC transport system substrate-binding protein polypeptide. In various embodiments the nucleic acid molecule comprises a promoter, including, for example, the lac, trc, trcRBS, phoA, tac, or λP_{L}/λP_{R} promoter from E. coli (or derivatives thereof) or the phoA, gpd, rplM, or rpsJ promoter from a coryneform bacteria.

In various embodiments, the polypeptide is a variant polypeptide with reduced feedback inhibition (e.g., relative to a wild-type form of the polypeptide). In various embodiments, the bacterium further comprises additional bacterial gene products involved in amino acid production. In various embodiments, the bacterium further comprises a nucleic acid molecule encoding a bacterial polypeptide described herein (e.g., a nucleic acid molecule encoding a bacterial homoserine dehydrogenase polypeptide). In various embodiments, the bacterium further comprises a nucleic acid molecule encoding a homologous bacterial polypeptide (i.e., a bacterial polypeptide that is native to the host species or a functional variant thereof), such as a bacterial polypeptide described herein. The homologous bacterial polypeptide can be expressed at high levels and/or conditionally expressed. For example, the nucleic acid encoding the homologous bacterial polypeptide can be operably linked to a promoter that allows expression of the polypeptide over wild-type levels, and/or the nucleic acid may be present in multiple copies in the bacterium.

In various embodiments the bacterial aspartokinase or functional variant thereof is chosen from: (a) a Mycobacterium smegmatis aspartokinase polypeptide or a functional variant thereof, (b) an Amycolatopsis mediterranei aspartokinase polypeptide or a functional variant thereof, (c) a Streptomyces coelicolor aspartokinase polypeptide or a functional variant thereof, (d) a Thermobifida fusca aspartokinase polypeptide or a functional variant thereof, (e) an Erwinia chrysanthemi aspartokinase polypeptide or a functional variant thereof, and (f) a Shewanella oneidensis aspartokinase polypeptide or a functional variant thereof. In certain embodiments, the bacterial aspartokinase polypeptide is an Escherichia coli aspartokinase polypeptide or a functional variant thereof. In certain embodiments, the bacterial aspartokinase polypeptide is a Corynebacterium glutamicum aspartokinase polypeptide or a functional variant thereof. In certain embodiments the bacterial asparatokinase polypeptide or functional variant thereof has reduced feedback inhibition.

In various embodiments the bacterial aspartate semialdehyde dehydrogenase polypeptide or functional variant thereof is chosen from: (a) a Mycobacterium smegmatis aspartate semialdehyde dehydrogenase polypeptide r a functional variant thereof, (b) an Amycolatopsis mediterranei aspartate semialdehyde dehydrogenase polypeptide or a functional variant thereof, (c) a Streptomyces coelicolor aspartate semialdehyde dehydrogenase polypeptide or a functional variant thereof, and (d) a Thermobifida fusca aspartate semialdehyde dehydrogenase polypeptide or a functional variant thereof. In certain embodiments, the bacterial aspartate semialdehyde dehydrogenase polypeptide is an Escherichia coli aspartate semialdehyde dehydrogenase polypeptide or a functional variant thereof. In certain embodiments, the bacterial aspartate semialdehyde dehydrogenase polypeptide is a Corynebacterium glutamicum aspartate semialdehyde dehydrogenase polypeptide or a functional variant thereof.

In various embodiments the bacterial phosphoenolpyruvate carboxylase polypeptide or functional variant thereof is chosen from: (a) a Mycobacterium smegmatis phosphoenolpyruvate carboxylase polypeptide or a functional variant thereof, (b) a Streptomyces coelicolor phosphoenolpyruvate carboxylase polypeptide or a functional variant thereof, (c) a Thermobifida fusca phosphoenolpyruvate carboxylase polypeptide or a functional variant thereof, and (d) an Erwinia chrysanthemi phosphoenolpyruvate carboxylase polypeptide or a functional variant thereof. In certain embodiments, the bacterial phosphoenolpyruvate carboxylase polypeptide is an Escherichia coli phosphoenolpyruvate carboxylase polypeptide or a functional variant thereof. In certain embodiments, the bacterial phosphoenolpyruvate carboxylase polypeptide is a Corynebacterium glutamicum phosphoenolpyruvate carboxylase polypeptide or a functional variant thereof.

In various embodiments the bacterial pyruvate carboxylase polypeptide or functional variant thereof is chosen from: (a) a Mycobacterium smegmatis pyruvate carboxylase polypeptide or a functional variant thereof, (b) a Streptomyces coelicolor pyruvate carboxylase polypeptide or a functional variant thereof, and (c) a Thermobifida fusca pyruvate carboxylase polypeptide or a functional variant thereof. In certain embodiments, the bacterial pyruvate carboxylase polypeptide is a Corynebacterium glutamicum pyruvate carboxylase or a functional variant thereof.

In various embodiments the bacterium is chosen from a coryneform bacterium or a bacterium of the family Enterobacteriaceae such as an Escherichia coli bacterium.

Coryneform bacteria include, without limitation, Corynebacterium glutamicum, Corynebacterium acetoglutamicum, Corynebacterium melassecola, Corynebacterium thermoaminogenes, Brevibacterium lactofermentum, Brevibacterium lactis, and Brevibacterium flavum.

In various embodiments, the Mycobacterium smegmatis aspartokinase polypeptide comprises at least one amino acid change chosen from: an alanine changed to a Group 1 amino acid residue at position 279; a serine changed to a Group 6 amino acid residue at position 301; a threonine changed to a Group 2 amino acid residue at position 311; and a glycine changed to a Group 3 amino acid residue at position 345; the Mycobacterium smegmatis aspartokinase comprises at least one amino acid change chosen from: an alanine changed to a proline at position 279, a serine changed to a tyrosine at position 301, a threonine changed to an isoleucine at position 311, and a 30 glycine changed to an aspartate at position 345.

In various embodiments, the Amycolatopsis mediterranei aspartokinase polypeptide comprises at least one amino acid change chosen from: an alanine changed to a Group 1 amino acid residue at position 279; a serine changed to a Group 6 amino acid residue at position 301 ; a threonine changed to a Group 2 amino acid residue at position 311; and a glycine changed to a Group 3 amino acid residue at position 345.

In various embodiments the Amycolatopsis mediterranei aspartokinase polypeptide comprises at least one amino acid change chosen from: an alanine changed to a proline at position 279; a serine changed to a tyrosine at position 301; a threonine changed to an isoleucine at position 311; and a glycine changed to an aspartate at position 345.

In various embodiments the Streptomyces coelicolor aspartokinase polypeptide comprises at least one amino acid change chosen from: an alanine changed to a Group 1 amino acid residue at position 282; a serine changed to a Group 6 amino acid residue at position 304; a serine changed to a Group 2 amino acid residue at position 314; and a glycine changed to a Group 3 amino acid residue at position 348.

In various embodiments the Streptomyces coelicolor aspartokinase polypeptide comprises at least one amino acid change chosen from: an alanine changed to a proline at position 282; a serine changed to a tyrosine at position 304; a serine changed to an isoleucine at position 314; and a glycine changed to an aspartate at position 348.

In various embodiments the Erwinia chrysanthemi aspartokinase polypeptide comprises at least one amino acid change chosen from: a glycine changed to a Group 3 amino acid residue at position 328; a leucine changed to a Group 6 amino acid residue at position 330; a serine changed to a Group 2 amino acid residue at position 350; and a valine changed to a Group 2 amino acid residue other than valine at position 352.

In various embodiments the Erwinia chrysanthemi aspartokinase polypeptide comprises at least one amino acid change chosen from: a glycine changed to an aspartate at position 328; a leucine changed to a phenylalanine at position 330; a serine changed to an isoleucine at position 350; and a valine changed to a methionine at position 352.

In various embodiments the Shewanella oneidensis aspartokinase polypeptide comprises at least one amino acid change chosen from: a glycine changed to a Group 3 amino acid residue at position 323; a leucine changed to a Group 6 amino acid residue at position 325; a serine changed to a Group 2 amino acid residue at position 345; and a valine changed to a Group 2 amino acid residue other than valine at position 347.

In various embodiments the Shewanella oneidensis aspartokinase polypeptide comprises at least one amino acid change chosen from: a glycine changed to an aspartate at position 323; a leucine changed to a phenylalanine at position 325; a serine changed to an isoleucine at position 345; and a valine changed to a methionine at position 347.

In various embodiments the Corynebacterium glutamicum aspartokinase polypeptide comprises at least one amino acid change chosen from: an alanine changed to a Group 1 amino acid other than alanine at position 279; a serine changed to a Group 6 amino acid residue at position 301; a threonine changed to a Group 2 amino acid residue at position 311; and a glycine changed to a Group 3 amino acid residue at position 345.

In various embodiments the Corynebacterium glutamicum aspartokinase polypeptide comprises at least one amino acid change chosen from: an alanine changed to a proline at position 279; a serine changed to a tyrosine at position 301; a threonine changed to an isoleucine at position 311; and a glycine changed to an aspartate at position 345.

In various embodiments the Escherichia coli aspartokinase polypeptide comprises at least one amino acid change chosen from: a glycine changed to a Group 3 amino acid residue at position 323; a leucine changed to a Group 6 amino acid residue at position 325; a serine changed to a Group 2 amino acid residue at position 345; and a valine changed to a Group 2 amino acid residue other than valine at position 347.

In various embodiments the Escherichia coli aspartokinase polypeptide comprises at least one amino acid change chosen from: a glycine changed to an aspartate at position 323; a leucine changed to a phenylalanine at position 325; a serine changed to an isoleucine at position 345; and a valine changed to a methionine at position 347.

In various embodiments, the Corynebacterium glutamicum pyruvate carboxylase polypeptide or variant thereof comprises a proline changed to Group 4 amino acid residue at position 458. In various embodiments, the Corynebacterium glutamicum pyruvate carboxylase polypeptide or variant thereof comprises a proline changed to a serine at position 458.

In various embodiments, the Mycobacterium smegmatis pyruvate carboxylase polypeptide or variant thereof comprises a proline changed to Group 4 amino acid residue at position 448. In various embodiments, the Mycobacterium smegmatis pyruvate carboxylase polypeptide or variant thereof comprises a proline changed to a serine at position 448.

In various embodiments, the Streptomyces coelicolor pyruvate carboxylase polypeptide or variant thereof comprises a proline changed to Group 4 amino acid residue at position 449. In various embodiments, the Streptomyces coelicolor pyruvate carboxylase polypeptide or variant thereof comprises a proline changed to a serine at position 449.

In various embodiments the bacterial dihydrodipicolinate synthase polypeptide or functional variant thereof is chosen from: a Mycobacterium smegmatis dihydrodipicolinate synthase polypeptide or a functional variant thereof; a Streptomyces coelicolor dihydrodipicolinate synthase polypeptide or a functional variant thereof; a Thermobifida fusca dihydrodipicolinate synthase polypeptide or a functional variant thereof; and an Erwinia chrysanthemi dihydrodipicolinate synthase polypeptide or a functional variant thereof. In certain embodiments, the bacterial dihydrodipicolinate synthase polypeptide or functional variant thereof with reduced feedback inhibition is an Escherichia coli dihydrodipicolinate synthase polypeptide or a functional variant thereof. In certain embodiments the bacterial dihydrodipicolinate synthase polypeptide or functional variant thereof has reduced feedback inhibition.

In various embodiments the Erwinia chrysanthemi dihydrodipicolinate synthase polypeptide comprises at least one amino acid change chosen from: an asparagine changed to a Group 2 amino acid residue at position 80; a leucine changed to a Group 6 amino acid residue at position 88; and a histidine changed to a Group 6 amino acid residue at position 18.

In various embodiments the Erwinia chrysanthemi dihydrodipicolinate synthase polypeptide comprises at least one amino acid change chosen from: an asparagine changed to an isoleucine at position 80; a leucine changed to a phenylalanine at position 88; and a histidine changed to a tyrosine at position 118.

In various embodiments, the Streptomyces coelicolor dihydrodipicolinate synthase polypeptide comprises at least one amino acid change chosen from: an asparagine changed to a Group 2 amino acid residue at position 89; a leucine changed to a Group 6 amino acid residue at position 97; and a histidine changed to a Group 6 amino acid residue at position 127.

In various embodiments the Streptomyces coelicolor dihydrodipicolinate synthase polypeptide comprises at least one amino acid change chosen from: an asparagine changed to an isoleucine at position 89; a leucine changed to a phenylalanine at position 97; and a histidine changed to a tyrosine at position 127.

In various embodiments the Mycobacterium smegmatis dihydrodipicolinate synthase polypeptide comprises at least one amino acid change chosen from: an amino acid residue corresponding to tyrosine 90 changed to a Group 2 amino acid residue; an amino acid residue corresponding to leucine 98 changed to a Group 6 amino acid residue; and an amino acid residue corresponding to histidine 128 changed to a Group 6 amino acid residue.

In various embodiments the Mycobacterium smegmatis dihydrodipicolinate synthase polypeptide comprises at least one amino acid change chosen from: an amino acid residue corresponding to tyrosine 90 changed to an isoleucine; an amino acid residue corresponding to leucine 98 changed to a phenylalanine; and an amino acid residue corresponding to histidine 128 changed to a histidine.

In various embodiments the Escherichia coli dihydrodipicolinate synthase polypeptide comprises at least one amino acid change chosen from: an asparagine changed to a Group 2 amino acid residue at position 80; an alanine changed to a Group 2 amino acid residue at position 81; a glutamatate changed to a Group 5 amino acid residue at position 84; a leucine changed to a Group 6 amino acid residue at position 88; and a histidine changed to a Group 6 amino acid at position 118.

In various embodiments the Escherichia coli dihydrodipicolinate synthase polypeptide comprises at least one amino acid change chosen from: an asparagine changed to an isoleucine at position 80; an alanine changed to a valine at position 81; a glutamate changed to a lysine at position 84; a leucine changed to a phenylalanine at position 88; and a histidine changed to a tyrosine at position 118.

In various embodiments the bacterial homoserine dehydrogenase polypeptide is chosen from: (a) a Mycobacterium smegmatis homoserine dehydrogenase polypeptide or functional variant thereof; (b) a Streptomyces coelicolor homoserine dehydrogenase polypeptide or a functional variant thereof; (c) a Thermobifida fusca homoserine dehydrogenase polypeptide or a functional variant thereof; and (d) an Erwinia chrysanthemi homoserine dehydrogenase polypeptide or a functional variant thereof. In certain embodiments, the bacterial homoserine dehydrogenase polypeptide is a homoserine dehydrogenase polypeptide from a coryneform bacteria or a functional variant thereof (e.g., a Corynebacterium glutamicum homoserine dehydrogenase polypeptide or functional variant thereof, or a Brevibacterium lactofermentum homoserine dehydrogenase polypeptide or functional variant thereof). In certain embodiments, the homoserine dehydrogenase polypeptide or functional variant thereof is an Escherichia coli homoserine dehydrogenase polypeptide or a functional variant thereof. In certain embodiments the homoserine dehydrogenase polypeptide or functional variant thereof has reduced feedback inhibition.

In various embodiments the Corynebacterium glutamicum or Brevibacterium lactofermentum homoserine dehydrogenase polypeptide comprises at least one amino acid change chosen from: a leucine change to a Group 6 amino acid residue at position 23; a valine changed to a Group 1 amino acid residue at position 59; a valine changed to another Group 2 amino acid residue at position 104; a glycine changed to Group 3 amino acid residue at position 378; and an alteration that truncates the homoserine dehydrogenase protein after the lysine amino acid residue at position 428.

In various embodiments the Corynebacterium glutamicum or Brevibacterium lactofermentum homoserine dehydrogenase polypeptide comprises at least one amino acid change chosen from: a leucine changed to a phenylalanine at position 23; valine changed to an alanine at position 59; a valine changed to an isoleucine at position 104; and a glycine changed to a glutamic acid at position 378.

In various embodiments the Mycobacterium smegmatis homoserine dehydrogenase polypeptide comprises at least one amino acid change chosen from: a valine change to a Group 6 amino acid residue at position 10; a valine changed to a Group 1 amino acid residue at position 46; and a glycine changed to Group 3 amino acid residue at position 364.

In various embodiments the Mycobacterium smegmatis homoserine dehydrogenase polypeptide comprises at least one amino acid change chosen from: a valine changed to a phenylalanine at position 10; valine changed to an alanine at position 46; and a glycine changed to a glutamic acid at position 378.

In various embodiments the Streptomyces coelicolor homoserine dehydrogenase polypeptide comprises at least one amino acid change chosen from: a leucine change to a Group 6 amino acid residue at position 10; a valine changed to a Group 1 amino acid residue at position 46; a glycine changed to Group 3 amino acid residue at position 362; an alteration that truncates the homoserine dehydrogenase protein after the arginine amino acid residue at position 412. In various embodiments the Streptomyces coelicolor homoserine dehydrogenase polypeptide comprises at least one amino acid change chosen from: a leucine changed to a phenylalanine at position 10; a valine changed to an alanine at position 46; and a glycine changed to a glutamic acid at position 362.

In various embodiments the Thermobifida fusca homoserine dehydrogenase polypeptide comprises at least one amino acid change chosen from: a leucine change to a Group 6 amino acid residue at position 192; a valine changed to a Group 1 amino acid residue at position 228; a glycine changed to Group 3 amino acid residue at position 545. In various embodiments, the Thermobifida fusca homoserine dehydrogenase polypeptide is truncated after the arginine amino acid residue at position 595.

In various embodiments the Thermobifida fusca homoserine dehydrogenase polypeptide comprises at least one amino acid change chosen from: a leucine changed to a phenylalanine at position 192; valine changed to an alanine at position 228; and a glycine changed to a glutamic acid at position 545.

In various embodiments the Escherichia coli homoserine dehydrogenase polypeptide comprises at least one amino acid change in SEQ ID NO:211 chosen from: a glycine changed to a Group 3 amino acid residue at position 330; and a serine changed to a Group 6 amino acid residue at position 352.

In various embodiments the Escherichia coli homoserine dehydrogenase polypeptide comprises at least one amino acid change in SEQ ID NO:211, chosen from: a glycine changed to an aspartate at position 330; and a serine changed to a phenylalanine at position 352.

In various embodiments the bacterial O-homoserine acetyltransferase polypeptide is chosen from: a Mycobacterium smegmatis O-homoserine acetyltransferase polypeptide or functional variant thereof; a Streptomyces coelicolor O-homoserine acetyltransferase polypeptide or a functional variant thereof; a Thermobifida fusca O-homoserine acetyltransferase polypeptide or a functional variant thereof; and an Erwinia chrysanthemi O-homoserine acetyltransferase polypeptide or a functional variant thereof. In certain embodiments, the bacterial O-homoserine acetyltransferase polypeptide is an O-homoserine acetyltransferase polypeptide from Corynebacterium glutamicum or a functional variant thereof. In certain embodiments the O-homoserine acetyltransferase polypeptide or functional variant thereof has reduced feedback inhibition.

In various embodiments the bacterial O-homoserine acetyltransferase polypeptide is a Thermobifida fusca O-homoserine acetyltransferase polypeptide or functional variant thereof; the Thermobifida fusca O-homoserine acetyltransferase polypeptide comprises SEQ ID NO:24 or a variant sequence thereof; the bacterial O-homoserine acetyltransferase polypeptide is a Corynebacterium glutamicum O-homoserine acetyltransferase polypeptide or functional variant thereof; the C. glutamicum O-homoserine acetyltransferase polypeptide comprises SEQ ID NO:212 or a variant sequence thereof; or the bacterial O-homoserine acetyltransferase polypeptide is a Escherichia coli O-homoserine acetyltransferase polypeptide or functional variant thereof; the Escherichia coli O-homoserine acetyltransferase polypeptide comprises SEQ ID NO:213 or a variant sequence thereof.

In various embodiments the bacterial O-acetylhomoserine sulfhydrylase polypeptide is chosen from: (a) a Mycobacterium smegmatis O-acetylhomoserine sulfhydrylase polypeptide or functional variant thereof; (b) a Streptomyces coelicolor O-acetylhomoserine sulfhydrylase polypeptide or a functional variant thereof; and (c) a Thermobifida fusca O-acetylhomoserine sulfhydrylase polypeptide or a functional variant thereof. In certain embodiments, the bacterial O-acetylhomoserine sulfhydrylase polypeptide is an O-acetylhomoserine sulfhydrylase polypeptide from Corynebacterium glutamicum or a functional variant thereof. In certain embodiments the O-acetylhomoserine sulfhydrylase polypeptide or functional variant thereof has reduced feedback inhibition.

In various embodiments the bacterial methionine adenosyltransferase polypeptide is chosen from: a Mycobacterium smegmatis methionine adenosyltransferase polypeptide or functional variant thereof; a Streptomyces coelicolor methionine adenosyltransferase polypeptide or a functional variant thereof; a Thermobifida fusca methionine adenosyltransferase polypeptide or a functional variant thereof; and an Erwinia chrysanthemi methionine adenosyltransferase polypeptide or a functional variant thereof. In certain embodiments, the bacterial methionine adenosyltransferase polypeptide is a methionine adenosyltransferase polypeptide from Corynebacterium glutamicum or a functional variant thereof. In certain embodiments, the bacterial methionine adenosyltransferase polypeptide is a methionine adenosyltransferase polypeptide from Escherichia coli or a functional variant thereof. In certain embodiments the methionine adenosyltransferase polypeptide or functional variant thereof has reduced feedback inhibition.

In various embodiments the Mycobacterium smegmatis methionine adenosyltransferase polypeptide comprises a valine change to a Group 3 amino acid residue at position 196. In various embodiments the Mycobacterium smegmatis methionine adenosyltransferase polypeptide comprises a valine change to a glutamic acid residue at position 196.

In various embodiments the Streptomyces coelicolor methionine adenosyltransferase polypeptide comprises a valine change to a Group 3 amino acid residue at position 195. In various embodiments the Streptomyces coelicolor methionine adenosyltransferase polypeptide comprises a valine change to a glutamic acid residue at position 195. In various embodiments the Thermobifida fusca methionine adenosyltransferase polypeptide comprises a valine change to a Group 3 amino acid residue at position 195. In various embodiments the Thermobifida fusca methionine adenosyltransferase polypeptide comprises a valine change to a glutamic acid residue at position 195.

In various embodiments the Erwinia chrysanthemi methionine adenosyltransferase polypeptide comprises a valine change to a Group 3 amino acid residue at position 185. In various embodiments the Erwinia chrysanthemi methionine adenosyltransferase polypeptide comprises a valine change to a glutamic acid residue at position 185.

In various embodiments the Corynebacterium glutamicum methionine adenosyltransferase polypeptide comprises a valine change to a Group 3 amino acid residue at position 200. In various embodiments the Corynebacterium glutamicum methionine adenosyltransferase polypeptide comprises a valine change to a glutamic acid residue at position 200.

In various embodiments the Escherichia coli methionine adenosyltransferase polypeptide comprises a valine change to a Group 3 amino acid residue at position 185. In various embodiments the Escherichia coli methionine adenosyltransferase polypeptide comprises a valine change to a glutamic acid residue at position 185.

In various embodiments the cobalamin-dependent methionine synthesis polypeptide (MetH) is a Mycobacterium smegmatis cobalamin-dependent methionine synthesis polypeptide or a functional variant thereof; a Streptomyces coelicolor cobalamin-dependent methionine synthesis polypeptide or a functional variant thereof; a Thermobifida fusca cobalamin-dependent methionine synthesis polypeptide or a functional variant thereof; an Erwinia chrysanthemi cobalamin-dependent methionine synthesis polypeptide or a functional variant thereof; an Escherichia coli cobalamin-dependent methionine synthesis polypeptide or a functional variant thereof; or a Corynebacterium glutamicum cobalamin-dependent methionine synthesis polypeptide or a functional variant thereof).

In various embodiments cobalamin-independent methionine synthesis polypeptide (MetE) is a Mycobacterium smegmatis cobalamin-independent methionine synthesis polypeptide or a functional variant thereof; a Streptomyces coelicolor cobalamin-independent methionine synthesis polypeptide or a functional variant thereof; a Thermobifida fusca cobalamin-independent methionine synthesis polypeptide or a functional variant thereof; an Erwinia chrysanthemi cobalamin-independent methionine synthesis polypeptide or a functional variant thereof; an Escherichia coli cobalamin-independent methionine synthesis polypeptide or a functional variant thereof; or a Corynebacterium glutamicum cobalamin-independent methionine synthesis polypeptide or a functional variant thereof).

In various embodiments the bacterium further comprises a nucleic acid molecule encoding a bacterial dihydrodipicolinate synthase polypeptide or a functional variant thereof.

In various embodiments the bacterial dihydrodipicolinate synthase polypeptide or a functional variant thereof is chosen from: a Mycobacterium smegmatis dihydrodipicolinate synthase polypeptide or a functional variant thereof; a Streptomyces coelicolor dihydrodipicolinate synthase polypeptide or a functional variant thereof; a Thermobifida fusca dihydrodipicolinate synthase polypeptide or a functional variant thereof; an Erwinia chrysanthemi dihydrodipicolinate synthase polypeptide or a functional variant thereof; an Escherichia coli dihydrodipicolinate synthase polypeptide or a functional variant thereof; and a Corynebacterium glutamicum dihydrodipicolinate synthase polypeptide or a functional variant thereof. In certain embodiments the dihydrodipicolinate synthase polypeptide or functional variant thereof has reduced feedback inhibition.

In various embodiments the bacterium further comprises at least one of: (a) a nucleic acid molecule (e.g., a recombinant nucleic acid molecule) encoding a bacterial homoserine dehydrogenase polypeptide or a functional variant thereof; (b) a nucleic acid molecule (e.g., a recombinant nucleic acid molecule) encoding a bacterial O-homoserine acetyltransferase polypeptide or a functional variant thereof; (c) a nucleic acid molecule (e.g., a recombinant nucleic acid molecule) encoding a O-acetylhomoserine sulfhydrylase polypeptide or a functional variant thereof. In certain embodiments one or more of the polypeptides or functional variants thereof has reduced feedback inhibition.

In various embodiments the bacterial homoserine dehydrogenase polypeptide is chosen from: a Mycobacterium smegmatis homoserine dehydrogenase polypeptide or functional variant thereof; a Streptomyces coelicolor homoserine dehydrogenase polypeptide or a functional variant thereof; a Thermobifida fusca homoserine dehydrogenase polypeptide or a functional variant thereof; an Escherichia coli homoserine dehydrogenase polypeptide or a functional variant thereof; a Corynebacterium glutamicum homoserine dehydrogenase polypeptide or a functional variant thereof; and an Erwinia chrysanthemi homoserine dehydrogenase polypeptide or a functional variant thereof. In certain embodiments the homoserine dehydrogenase polypeptide or functional variant thereof has reduced feedback inhibition.

In various embodiments the bacterial O-homoserine acetyltransferase polypeptide is chosen from: a Mycobacterium smegmatis O-homoserine acetyltransferase polypeptide or functional variant thereof; a Streptomyces coelicolor O-homoserine acetyltransferase polypeptide or a functional variant thereof; a Thermobifida fusca O-homoserine acetyltransferase polypeptide or a functional variant thereof; an Erwinia chrysanthemi O-homoserine acetyltransferase polypeptide or a functional variant thereof; an Escherichia coli O-homoserine acetyltransferase polypeptide or a functional variant thereof; and a Corynebacterium glutamicum O-homoserine acetyltransferase polypeptide or a functional variant thereof. In certain embodiments the O-homoserine acetyltransferase polypeptide or functional variant thereof has reduced feedback inhibition.

In various embodiments the bacterial O-acetylhomoserine sulfhydrylase polypeptide is chosen from: a Mycobacterium smegmatis O-acetylhomoserine sulfhydrylase or functional variant thereof; a Streptomyces coelicolor O-acetylhomoserine sulfhydrylase polypeptide or a functional variant thereof; a Thermobifida fusca O-acetylhomoserine sulfhydrylase polypeptide or a functional variant thereof; and a Corynebacterium glutamicum O-acetylhomoserine sulfhydrylase polypeptide or a functional variant thereof. In certain embodiments the O-acetylhomoserine sulfhydrylase polypeptide or functional variant thereof has reduced feedback inhibition.

In various embodiments the bacterium further comprises a nucleic acid molecule (e.g., a recombinant nucleic acid molecule) encoding a bacterial methionine adenosyltransferase polypeptide (e.g., a Mycobacterium smegmatis methionine adenosyltransferase polypeptide or functional variant thereof; a Streptomyces coelicolor methionine adenosyltransferase polypeptide or a functional variant thereof; a Thermobifida fusca methionine adenosyltransferase polypeptide or a functional variant thereof; an Erwinia chrysanthemi methionine adenosyltransferase polypeptide or a functional variant thereof; an Escherichia coli methionine adenosyltransferase polypeptide or a functional variant thereof; or a Corynebacterium glutamicum methionine adenosyltransferase polypeptide or a functional variant thereof).

In various embodiments the bacterium further comprises a nucleic acid molecule (e.g., a recombinant nucleic acid molecule) encoding a cobalamin-dependent methionine synthesis polypeptide (MetH) (e.g., a Mycobacterium smegmatis cobalamin-dependent methionine synthesis polypeptide or functional variant thereof; a Streptomyces coelicolor cobalamin-dependent methionine synthesis polypeptide or a functional variant thereof; a Thermobifida fusca cobalamin-dependent methionine synthesis polypeptide or a functional variant thereof; an Erwinia chrysanthemi cobalamin-dependent methionine synthesis polypeptide or a functional variant thereof; an Escherichia coli methionine cobalamin-dependent methionine synthesis polypeptide or a functional variant thereof; or a Corynebacterium glutamicum cobalamin-dependent methionine synthesis polypeptide or a functional variant thereof).

In various embodiments the bacterium further comprises a nucleic acid molecule (e.g., a recombinant nucleic acid molecule) encoding a cobalamin-independent methionine synthesis polypeptide (MetE) (e.g., a Mycobacterium smegmatis cobalamin-independent methionine synthesis polypeptide or functional variant thereof; a Streptomyces coelicolor cobalamin-dependent methionine synthesis polypeptide or a functional variant thereof; a Thermobifida fusca cobalamin-independent methionine synthesis polypeptide or a functional variant thereof; an Erwinia chrysanthemi cobalamin-independent methionine synthesis polypeptide or a functional variant thereof; an Escherichia coli methionine cobalamin-independent methionine synthesis polypeptide or a functional variant thereof; or a Corynebacterium glutamicum cobalamin-independent methionine synthesis polypeptide or a functional variant thereof).

In various embodiments the bacterial glycine dehydrogenase (decarboxylating) polypeptide is chosen from: (a) an E. coli glycine dehydrogenase (decarboxylating) polypeptide or functional variant thereof; (b) a B. halodurans glycine dehydrogenase (decarboxylating) polypeptide or a functional variant thereof; (c) a T. fusca glycine dehydrogenase (decarboxylating) polypeptide or a functional variant thereof; (d) an E. carotovora glycine dehydrogenase (decarboxylating) polypeptide or a functional variant thereof; and (e) an S. coelicolor glycine dehydrogenase (decarboxylating) polypeptide or a functional variant thereof.

In various embodiments the bacterial H polypeptide (involved in the glycine cleavage system) is chosen from: (a) an E. coli H polypeptide (involved in the glycine cleavage system) or functional variant thereof; (b) a B. halodurans H polypeptide (involved in the glycine cleavage system) or a functional variant thereof; (c) a T. fusca H polypeptide (involved in the glycine cleavage system) or a functional variant thereof; (d) an E. carotovora H polypeptide (involved in the glycine cleavage system) or a functional variant thereof; and (e) an S. coelicolor H polypeptide (involved in the glycine cleavage system) or a functional variant thereof.

In various embodiments the bacterial aminomethyl transferase polypeptide is chosen from: (a) an E. coli aminomethyl transferase polypeptide or functional variant thereof; (b) a B. halodurans aminomethyl transferase polypeptide or a functional variant thereof; (c) a T. fusca aminomethyl transferase polypeptide or a functional variant thereof; (d) an E. carotovora aminomethyl transferase polypeptide or a functional variant thereof; and (e) an S. coelicolor aminomethyl transferase polypeptide or a functional variant thereof. In certain embodiments, the bacterial aminomethyl transferase polypeptide is an aminomethyl transferase polypeptide from Corynebacterium glutamicum or a functional variant thereof.

In various embodiments the bacterial dihydrolipoamide dehydrogenase polypeptide is chosen from: (a) an E. coli dihydrolipoamide dehydrogenase polypeptide or functional variant thereof; (b) a B. halodurans dihydrolipoamide dehydrogenase polypeptide or a functional variant thereof; (c) a T. fusca dihydrolipoamide dehydrogenase polypeptide or a functional variant thereof; (d) an E. carotovora dihydrolipoamide dehydrogenase polypeptide or a functional variant thereof; and (e) an S. coelicolor dihydrolipoamide dehydrogenase polypeptide or a functional variant thereof. In certain embodiments, the bacterial dihydrolipoamide dehydrogenase polypeptide is a dihydrolipoamide dehydrogenase polypeptide from Corynebacterium glutamicum or a functional variant thereof.

In various embodiments the bacterial lipoic acid synthase polypeptide is chosen from: (a) an E. coli lipoic acid synthase polypeptide or functional variant thereof; (b) a B. halodurans lipoic acid synthase polypeptide or a functional variant thereof; (c) a T. fusca lipoic acid synthase polypeptide or a functional variant thereof; (d) an E. carotovora lipoic acid synthase polypeptide or a functional variant thereof; and (e) an S. coelicolor lipoic acid synthase polypeptide or a functional variant thereof. In certain embodiments, the bacterial lipoic acid synthase polypeptide is a lipoic acid synthase polypeptide from Corynebacterium glutamicum or a functional variant thereof.

In various embodiments the bacterial lipoyl-[acyl-carrier-protein]-protein-N-lipoyltransferase polypeptide is chosen from: (a) an E. coli lipoyl-[acyl-carrier-protein]-protein-N-lipoyltransferase polypeptide or functional variant thereof; (b) a T. fusca lipoyl-[acyl-carrier-protein]-protein-N-lipoyltransferase polypeptide or a functional variant thereof; (c) an E. carotovora lipoyl-[acyl-carrier-protein]-protein-N-lipoyltransferase polypeptide or a functional variant thereof; and (d) an S. coelicolor lipoyl-[acyl-carrier-protein]-protein-N-lipoyltransferase polypeptide or a functional variant thereof. In certain embodiments, the bacterial lipoyl-[acyl-carrier-protein]-protein-N-lipoyltransferase polypeptide is a lipoyl-[acyl-carrier-protein]-protein-N-lipoyltransferase polypeptide from Corynebacterium glutamicum or a functional variant thereof.

In various embodiments the bacterial lipoate-protein ligase A polypeptide is chosen from: (a) an E. coli lipoate-protein ligase A polypeptide or functional variant thereof; (b) a B. halodurans lipoate-protein ligase A polypeptide or a functional variant thereof; and (c) an S. coelicolor lipoate-protein ligase A polypeptide or a functional variant thereof. In certain embodiments, the bacterial lipoate-protein ligase A polypeptide is a lipoate-protein ligase A polypeptide from Corynebacterium glutamicum or a functional variant thereof.

In various embodiments, the bacterial fructose 1,6 bisphosphatase polypeptide is chosen from: (a) an E. coli fructose 1,6 bisphosphatase polypeptide or functional variant thereof; (b) a B. halodurans fructose 1,6 bisphosphatase polypeptide or a functional variant thereof; (c) an S. coelicolor fructose 1,6 bisphosphatase polypeptide or a functional variant thereof, (d) a C. acetobutylicum fructose 1,6 bisphosphatase polypeptide or a functional variant thereof, (e) an E. carotovora fructose 1,6 bisphosphatase polypeptide or a functional variant thereof, (f) an M. Smegmatis fructose 1,6 bisphosphatase polypeptide or a functional variant thereof, and (g) a T. fusca fructose 1,6 bisphosphatase polypeptide or a functional variant thereof. In certain embodiments, the bacterial fructose 1,6 bisphosphatase polypeptide is a fructose 1,6 bisphosphatase polypeptide from Corynebacterium glutamicum or a functional variant thereof.

In various embodiments, glucose 6 phosphate dehydrogenase polypeptide is chosen from: (a) an E. coli glucose 6 phosphate dehydrogenase polypeptide or functional variant thereof; (b) an S. coelicolor glucose 6 phosphate dehydrogenase polypeptide or a functional variant thereof, (c) an E. carotovora glucose 6 phosphate dehydrogenase polypeptide or a functional variant thereof, (d) an M. Smegmatis glucose 6 phosphate dehydrogenase polypeptide or a functional variant thereof, and (e) a T. fusca glucose 6 phosphate dehydrogenase polypeptide or a functional variant thereof. In certain embodiments, the bacterial glucose 6 phosphate dehydrogenase polypeptide is a glucose 6 phosphate dehydrogenase polypeptide from Corynebacterium glutamicum or a functional variant thereof.

In various embodiments, the bacterial glucose-6-phosphate isomerase polypeptide is chosen from: (a) an E. coli glucose-6-phosphate isomerase polypeptide or functional variant thereof; (b) a B. halodurans glucose-6-phosphate isomerase polypeptide or a functional variant thereof; (c) an S. coelicolor glucose-6-phosphate isomerase polypeptide or a functional variant thereof, (d) a C. acetobutylicum glucose-6-phosphate isomerase polypeptide or a functional variant thereof, (e) an E. carotovora glucose-6-phosphate isomerase polypeptide or a functional variant thereof, (f) an M. Smegmatis glucose-6-phosphate isomerase polypeptide or a functional variant thereof, and (g) a T. fusca glucose-6-phosphate isomerase polypeptide or a functional variant thereof. In certain embodiments, the bacterial glucose-6-phosphate isomerase polypeptide is a glucose-6-phosphate isomerase polypeptide from Corynebacterium glutamicum or a functional variant thereof.

In various embodiments, the bacterial NCgl2640 polypeptide is chosen from: (a) an E. coli NCgl2640 polypeptide or functional variant thereof; (b) an S. coelicolor NCgl2640 polypeptide or a functional variant thereof, and (c) a T. fusca NCgl2640 polypeptide or a functional variant thereof. In certain embodiments, the bacterial NCgl2640 polypeptide is an NCgl2640 polypeptide polypeptide from Corynebacterium glutamicum or a functional variant thereof.

Also featured is a coryneform bacterium or a bacterium of the family Enterobacteriaceae such as an Escherichia coli bacterium comprising at least two of: (a) a nucleic acid molecule encoding a bacterial homoserine dehydrogenase polypeptide or a functional variant thereof, (b) a nucleic acid molecule encoding a bacterial O-homoserine acetyltransferase polypeptide or a functional variant thereof; and (c) a nucleic acid molecule encoding a bacterial O-acetylhomoserine sulfhydrylase polypeptide or a functional variant thereof. In certain embodiments one or more of the bacterial polypetides or functional variants thereof has reduced feedback inhibition

In various embodiments, the bacterium has reduced activity of one or more of the following polypeptides, relative to a control: (a) a phosphoenolpyruvate carboxykinase polypeptide; and (b) an mcbR gene product polypeptide, e.g., the bacterium comprises a mutation in an endogenous pck gene or an endogenous mcbR gene, e.g., the bacterium comprises a mutation in an endogenous pck gene and an endogenous mcbR gene.

Also described is a method of producing an amino acid or a related metabolite, the method comprising: cultivating (i.e., culturing in a culture medium) a bacterium (e.g., a bacterium described herein) under conditions that allow the amino acid the metabolite to be produced, and collecting a composition that comprises the amino acid or related metabolite from the culture (the composition can be essentially cell free culture medium in which the cells have been cultured or can contain cells or can contain cell debris, e.g., lysed cells or can be essentially cells). The method can further include fractionating at least a portion of the collected composition (or culture) to obtain a fraction enriched in the amino acid or the metabolite.

The fraction can be further treated to create a composition that is at least 10%, 20%, 30%, 40%, 50%, 60%, 70%, 80%, 90%, 95%, 98% or 99% by weight the amino acid or related metabolite.

Also described is a method for producing an amino acid (e.g., methionine, lysine, threonine, isoleucine, S-adenosyl methionine), the method comprising: cultivating a bacterium described herein under conditions that allow the amino acid to be produced, and collecting the culture. The culture can be fractionated (e.g., to remove cells and/or to obtain fractions enriched in the amino acid).

Further featured is a method for the preparation of an amino acid or metabolite or a product containing an amino acid or metabolite, the method comprising two or more of the following steps:
(a) cultivating a bacterium (e.g., a bacterium described herein) under conditions that allow the amino acid or metabolite to be produced;
(b) collecting a composition that comprises at least a portion of the amino acid or metabolite
(c) concentrating of the collected composition to enrich for the amino acid or metabolite; and
(d) optionally, adding of one or more substances to obtain a desired product.

In the case of animal feed products containing an amino acid or metabolite the substances that can be added include, but are not limited to, e.g., conventional organic or inorganic auxiliary substances or carriers, such as gelatin, cellulose derivatives (e.g., cellulose ethers), silicas, silicates, stearates, grits, brans, meals, starches, gums, alginates sugars or others, and/or mixed and stabilized with conventional thickeners or binders.

In various embodiments, the composition that is collected lacks bacterial cells. In various embodiments, the composition that is collected contains less than 10%, 5%, 1%, 0.5% of the bacterial cells that result from cultivating the bacterium. In various embodiments, the composition comprises at least 1% (e.g., at least 1%, 5%, 10%, 20%, 40%, 50%, 75%, 80%, 90%, 95%, or to 100%) of the bacterial cells that result from cultivating the bacterium.

Described here in are Enterobacteriaceae or coryneform bacterium comprising at least one isolated nucleic acid molecule selected from the group consisting of:

(a) a nucleic acid molecule comprising a sequence encoding a bacterial sulfate ABC transporter ATP-binding polypeptide or a functional variant thereof;

(b) a nucleic acid molecule comprising a sequence encoding a bacterial sulfate transport system permease W polypeptide or a functional variant thereof;

(c) a nucleic acid molecule comprising a sequence encoding a bacterial sulfate, thiosulfate transport system permease T polypeptide or a functional variant thereof;

(d) a nucleic acid molecule comprising a sequence encoding a bacterial sulfate adenylyltransferase subunit 1 polypeptide or a functional variant thereof;

(e) a nucleic acid molecule comprising a sequence encoding a bacterial sulfate adenylyltransferase subunit 2 polypeptide or a functional variant thereof;

(f) a nucleic acid molecule comprising a sequence encoding a bacterial adenylylsulfate kinase polypeptide or a functional variant thereof;

(g) a nucleic acid molecule comprising a sequence encoding a bacterial phosphoadenosine phosphosulfate reductase polypeptide or a functional variant thereof;

(h) a nucleic acid molecule comprising a sequence encoding a bacterial sulfite reductase alpha subunit polypeptide or a functional variant thereof;

(i) a nucleic acid molecule comprising a sequence encoding a bacterial sulfite reductase hemopolypeptide beta-component polypeptide or a functional variant thereof;

(j) a nucleic acid molecule comprising a sequence encoding a bacterial sulfite reductase (NADPH), flavopolypeptide beta subunit polypeptide or a functional variant thereof;

(k) a nucleic acid molecule comprising a sequence encoding a bacterial adenylyl-sulphate reductase alpha subunit polypeptide or a functional variant thereof;

(l) a nucleic acid molecule comprising a sequence encoding a bacterial phosphoglycerate dehydrogenase polypeptide or a functional variant thereof;

(m) a nucleic acid molecule comprising a sequence encoding a bacterial phosphoserine transaminase polypeptide or a functional variant thereof;

(n) a nucleic acid molecule comprising a sequence encoding a bacterial phosphoserine phosphatase polypeptide or a functional variant thereof;

(o) a nucleic acid molecule comprising a sequence encoding a bacterial serine O-acetyltransferase polypeptide or a functional variant thereof;

(p) a nucleic acid molecule comprising a sequence encoding a bacterial cysteine synthase A polypeptide or a functional variant thereof;

(q) a nucleic acid molecule comprising a sequence encoding a bacterial cysteine synthase B polypeptide or a functional variant thereof;

(r) a nucleic acid molecule comprising a sequence encoding a bacterial ABC-type vitamin B12 transporter permease component polypeptide or a functional variant thereof;

(s) a nucleic acid molecule comprising a sequence encoding a bacterial ABC-type vitamin B12 transporter ATPase component polypeptide or a functional variant thereof;

(t) a nucleic acid molecule comprising a sequence encoding a bacterial ABC-type cobalamin/Fe3+-siderophore transport system polypeptide or a functional variant thereof;

(u) a nucleic acid molecule comprising a sequence encoding a bacterial adenosyltransferase polypeptide or a functional variant thereof;

(v) a nucleic acid molecule comprising a sequence encoding a bacterial GTP cyclohydrolase I polypeptide or a functional variant thereof;

(w) a nucleic acid molecule comprising a sequence encoding a bacterial phoA, psiA, or psiF gene product polypeptide or a functional variant thereof;

(x) a nucleic acid molecule comprising a sequence encoding a bacterial dihydroneopterin aldolase polypeptide or a functional variant thereof;

(y) a nucleic acid molecule comprising a sequence encoding a bacterial 7,8-dihydro-6-hydroxymethylpterin-pyrophosphokinase polypeptide or a functional variant thereof;

(z) a nucleic acid molecule comprising a sequence encoding a bacterial dihydropteroate synthase polypeptide or a functional variant thereof;

(aa) a nucleic acid molecule comprising a sequence encoding a bacterial dihydrofolate synthetase polypeptide or a functional variant thereof;

(ab) a nucleic acid molecule comprising a sequence encoding a bacterial dihydrofolate reductase polypeptide or a functional variant thereof;

(ac) a nucleic acid molecule comprising a sequence encoding a bacterial folylpolyglutamate synthetase polypeptide or a functional variant thereof;

(ad) a nucleic acid molecule comprising a sequence encoding a putative bacterial methionine (APC transporter superfamily) permease (YjeH) polypeptide or a functional variant thereof;

(ae) a nucleic acid molecule comprising a sequence encoding a bacterial transcriptional activator of MetE/H polypeptide or a functional variant thereof;

(af) a nucleic acid molecule comprising a sequence encoding a bacterial 6-phosphogluconate dehydrogenase polypeptide or a functional variant thereof;

(ag) a nucleic acid molecule comprising a sequence encoding a bacterial S-methylmethionine homocysteine methyltransferase polypeptide or a functional variant thereof;

(ah) a nucleic acid molecule comprising a sequence encoding a bacterial S-adenosylhomocysteine hydrolase polypeptide or a functional variant thereof;

(ai) a nucleic acid molecule comprising a sequence encoding a bacterial site-specific DNA methylase polypeptide or a functional variant thereof;

(aj) a nucleic acid molecule comprising a sequence encoding a bacterial methionine export sytem protein 1 polypeptide or a functional variant thereof;

(ak) a nucleic acid molecule comprising a sequence encoding a bacterial methionine export sytem protein 2 polypeptide or a functional variant thereof;

(al) a nucleic acid molecule comprising a sequence encoding a bacterial ABC transport system ATP-binding protein (MetN) polypeptide or a functional variant thereof;

(am) a nucleic acid molecule comprising a sequence encoding a bacterial ABC transport system permease protein (MetP) polypeptide or a functional variant thereof;

(an) a nucleic acid molecule comprising a sequence encoding a bacterial ABC transport system substrate-binding protein (MetQ) polypeptide or a functional variant thereof;

(ao) a nucleic acid molecule comprising a sequence encoding a bacterial aspartokinase polypeptide or a functional variant thereof;

(ap) a nucleic acid molecule comprising a sequence encoding a bacterial aspartate semialdehyde dehydrogenase or a functional variant thereof;

(aq) a nucleic acid molecule comprising a sequence encoding a bacterial homoserine dehydrogenase polypeptide or a functional variant thereof;

(ar) a nucleic acid molecule comprising a sequence encoding a bacterial O-homoserine acetyl transferase polypeptide or a functional variant thereof;

(as) a nucleic acid molecule comprising a sequence encoding a bacterial O-acetylhomoserine sulfhydrylase polypeptide or a functional variant thereof;

(at) a nucleic acid molecule comprising a sequence encoding a bacterial cobalamin-dependent methionine synthase polypeptide or a functional variant thereof;

(au) a nucleic acid molecule comprising a sequence encoding a bacterial cobalamin-independent methionine synthase polypeptide or a functional variant thereof;

(av) a nucleic acid molecule comprising a sequence encoding a bacterial homoserine kinase polypeptide or a functional variant thereof;

(aw) a nucleic acid molecule comprising a sequence encoding a bacterial methionine adenosyltransferase polypeptide or a functional variant thereof;

(ax) a nucleic acid molecule comprising a sequence encoding a bacterial O-succinylhomoserine (thio)-lyase polypeptide or a functional variant thereof;

(ay) a nucleic acid molecule comprising a sequence encoding a bacterial cystathionine beta-lyase polypeptide or a functional variant thereof;

(az) a nucleic acid molecule comprising a sequence encoding a bacterial 5,10-methylenetetrahydrofolate reductase polypeptide or a functional variant thereof;

(ba) a nucleic acid molecule comprising a sequence encoding a bacterial dihydrodipicolinate synthase polypeptide or a functional variant thereof;

(bb) a nucleic acid molecule comprising a sequence encoding a bacterial pyruvate carboxylase polypeptide or a functional variant thereof;

(bc) a nucleic acid molecule comprising a sequence encoding a bacterial glutamate dehydrogenase polypeptide or a functional variant thereof;

(bd) a nucleic acid molecule comprising a sequence encoding a bacterial diaminopimelate dehydrogenase polypeptide or a functional variant thereof;

(be) a nucleic acid molecule comprising a sequence encoding a bacterial methionine and cysteine biosynthesis repressor (McbR) polypeptide or a functional variant thereof;

(bf) a nucleic acid molecule comprising a sequence encoding a bacterial lysine exporter protein polypeptide or a functional variant thereof;

(bg) a nucleic acid molecule comprising a sequence encoding a bacterial phosphoenolpyruvate carboxykinase polypeptide or a functional variant thereof;

(bh) a nucleic acid molecule comprising a sequence encoding a bacterial phosphoenolpyruvate carboxylase polypeptide or a functional variant thereof;

(bi) a nucleic acid molecule comprising a sequence encoding a bacterial glycine dehydrogenase (decarboxylating) polypeptide or a functional variant thereof;

(bj) a nucleic acid molecule comprising a sequence encoding a bacterial H polypeptide (involved in the glycine cleavage system) or a functional variant thereof;

(bk) a nucleic acid molecule comprising a sequence encoding a bacterial aminomethyl transferase polypeptide or a functional variant thereof;

(bl) a nucleic acid molecule comprising a sequence encoding a bacterial dihydrolipoamide dehydrogenase polypeptide or a functional variant thereof;

(bm) a nucleic acid molecule comprising a sequence encoding a bacterial lipoate-protein ligase A polypeptide or a functional variant thereof;

(bn) a nucleic acid molecule comprising a sequence encoding a bacterial lipoic acid synthase polypeptide or a functional variant thereof;

(bo) a nucleic acid molecule comprising a sequence encoding a bacterial lipoyl-[acyl-carrier-protein]-protein-N-lipoyltransferase polypeptide or a functional variant thereof;

(bp) a nucleic acid molecule comprising a sequence encoding a bacterial fructose 1,6 bisphosphatase polypeptide or a functional variant thereof;

(bq) a nucleic acid molecule comprising a sequence encoding a bacterial glucose 6 phosphate dehydrogenase polypeptide or a functional variant thereof;

(br) a nucleic acid molecule comprising a sequence encoding a glucose-6-phosphate isomerase polypeptide or a functional variant thereof; and

(bs) a nucleic acid molecule comprising a sequence encoding a bacterial NCgl2640 polypeptide or a functional variant thereof; and

all combinations and subcombinations of (a)-(bs).

Also described herein are bacterium wherein: the bacterium comprises at least two of nucleic acid molecules (a)-(bs); the bacterium comprises at least three of nucleic acid molecules (a)-(bs); the bacterium comprises at least four of nucleic acid molecules (a)-(bs); the bacterium comprises at least five of nucleic acid molecules (a)-(bs); at least one of the polypeptides is heterologous to the bacterium; at least two of the polypeptides are heterologous to the bacterium; the bacterium is an Escherichia coli bacterium; the bacterium is a Corynebacterium glutamicum bacterium; the polypeptide (i.e., the polypeptide of any of (a)-(bs)) is selected from an Enterobacteriaceae polypeptide, an Actinomycete polypeptide, or a variant thereof; the polypeptide (i.e., the polypeptide of any of (a)-(bs)) is a polypeptide of one of the following Actinomycetes species: Mycobacterium smegmatis, Streptomyces coelicolor, Thermobifida fusca, Amycolatopsis mediterranei, Nocardia farcinica, and coryneform bacteria, including Corynebacterium glutamicum and Corynebacterium diphtheriae; the polypeptide (i.e., the polypeptide of any of (a)-(bs)) is from one or more of Mycobacterium smegmatis, Streptomyces coelicolor and Thermobifida fusca; the Enterobacteriaceae or coryneform bacterium (host strain) comprising the nucleic acid molecule is C. glutamicum; the Enterobacteriaceae or coryneform bacterium (host strain) comprising the nucleic acid molecule is Erwinia chysanthemi or Escherichia coli.

Also described are any of the forgoing bacterium wherein the bacterium has reduced activity or expression of one or more of the following polypeptides relative to the bacterium prior to any genetic modifications: a dihydrodipicolinate synthase polypeptide; an mcbR gene product polypeptide; a homoserine dehydrogenase polypeptide, a homoserine kinase polypeptide, a methionine adenosyltransferase polypeptide, a homoserine O-acetyltransferase polypeptide, a phosphoenolpyruvate carboxykinase polypeptide, an adenosyl transferase polypeptide, a diaminopimelate dehydrogenase polypeptide, an ABC transport system ATP-binding protein polypeptide, an ABC transport system permease protein polypeptide, glucose-6-phosphate isomerase, an NCgl2640 polypeptide, and an ABC transport system substrate-binding protein polypeptide. The bacterium can have reduced activity of any of the various combinations and sub-combinations of these polypeptides.

Also described are any of the forgoing bacterium wherein: the bacterium comprises (a) and at least one of: (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (b) and at least one of (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (c) and at least one of (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (d) and at least one of (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (e) and at least one of (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (f) and at least one of (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (g) and at least one of (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (h) and at least one of (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (i) and at least one of (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (j) and at least one of (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (k) and at least one of (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (l) and at least one of (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (m) and at least one of (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (n) and at least one of (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); bacterium comprises (o) and at least one of (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (p) and at least one of (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (q) and at least one of (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (r) and at least one of (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (s) and at least one of (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (t) and at least one of (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (u) and at least one of (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (v) and at least one of (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (w) and at least one of (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (x) and at least one of (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (y) and at least one of (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (z) and at least one of (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (aa) and at least one of (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ab) and at least one of (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ac) and at least one of (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq); (br), and (bs); the bacterium comprises (ad) and at least one of (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ae) and at least one of (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (af) and at least one of (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ag) and at least one of (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ah) and at least one of (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ai) and at least one of (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (aj) and at least one of (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ak) and at least one of (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (al) and at least one of (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (am) and at least one of (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (an) and at least one of (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ao) and at least one of (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ap) and at least one of (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (aq) and at least one of (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ar) and at least one of (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (as) and at least one of (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (at) and at least one of (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (au) and at least one of (av), (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (av) and at least one of (aw), (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (aw) and at least one of (ax), (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ax) and at least one of (ay), (az), (ba), (bb), (bc), (bd), (be), (bf), (bg), (bh), (bi), (bj), (bk), (bl), (bm), (bn), (bo), (bp), (bq), (br), and (bs); the bacterium comprises (ay) and at least one of (az), (ba), (bb), (bc), (bd), (be), (bf), (bg), (bh), (bi), (bj), (bk), (bl), (bm), (bn), (bo), (bp), (bq), (br), and (bs); the bacterium comprises (az) and at least one of (ba), (bb), (bc), (bd), (be), (bf), (bg), (bh), (bi), (bj), (bk), (bl), (bm), (bn), (bo), (bp), (bq), (br), and (bs); the bacterium comprises (ba) and at least one of (bb), (bc), (bd), (be), (bf), (bg), (bh), (bi), (bj), (bk), (bl), (bm), (bn), (bo), (bp), (bq), (br), and (bs); the bacterium comprises (bb) and at least one of (bc), (bd), (be), (bf), (bg), (bh), (bi), (bj), (bk), (bl), (bm), (bn), (bo), (bp), (bq), (br), and (bs); the bacterium comprises (bc) and at least one of (bd), (be), (bf), (bg), (bh), (bi), (bj), (bk), (bl), (bm), (bn), (bo), (bp), (bq), (br), and (bs); the bacterium comprises (bd) and at least one of (be), (bf), (bg), (bh), (bi), (bj), (bk), (bl), (bm), (bn), (bo), (bp), (bq), (br), and (bs); the bacterium comprises (be) and at least one of (bf), (bg), (bh), (bi), (bj), (bk), (bl), (bm), (bn), (bo), (bp), (bq), (br), and (bs); the bacterium comprises (bf) and at least one of (bg), (bh), (bi), (bj), (bk), (bl), (bm), (bn), (bo), (bp), (bq), (br), and (bs); the bacterium comprises (bg) and at least one of (bh), (bi), (bj), (bk), (bl), (bm), (bn), (bo), (bp), (bq), (br), and (bs); the bacterium comprises (bh) and at least one of (bi), (bj), (bk), (bl), (bm), (bn), (bo), (bp), (bq), (br), and (bs); the bacterium comprises (bi) and at least one of (bj), (bk), (bl), (bm), (bn), (bo), (bp), (bq), (br), and (bs); the bacterium comprises (bj) and at least one of (bk), (bl), (bm), (bn), (bo), (bp), (bq), (br), and (bs); the bacterium comprises (bk) and at least one of (bl), (bm), (bn), (bo), (bp), (bq), (br), and (bs); the bacterium comprises (bl) and at least one of (bm), (bn), (bo), (bp), (bq), (br), and (bs); the bacterium comprises (bm) and at least one of (bn), (bo), (bp), (bq), (br), and (bs); the bacterium comprises (bn) and and at least one of (bo), (bp), (bq), (br), and (bs); the bacterium comprises (bo) and and at least one of (bp), (bq), (br), and (bs); the bacterium comprises (bp) and and at least one of (bq), (br), and (bs); the bacterium comprises (bq) and at least one of (br), and (bs); the bacterium comprises (br) and (bs); the bacterium comprises (aj) and (ak).

Also described are bacterium wherein: the bacterium comprises (r), (s) and (t); the bacterium comprises (a), (b) and (c); the bacterium comprises (d) and (e); the bacterium comprises (i) and (j); the bacterium comprises (l) and (o); the bacterium comprises (p) and (q); the bacterium comprises (bi), (bj), and (bk); the bacterium comprises (bi), (bj), (bk) and (bl); the bacterium comprises (bi), (bj), (bk) and at least one of: (1) (bm) or (2) (bn) and (o); and the bacterium comprises (bi), (bj), (bk) (bl) and at least one of: (1) (bm) or (2) (bn) and (bo).

Also described is: a bacterium comprising at least one isolated nucleic acid molecule selected from the group consisting of (a)-(an) and at least one isolated nucleic acid molecule selected from the group consisting of (ao)-(bs); a bacterium comprising at least one isolated nucleic acid molecule selected from the group consisting of (a)-(an) and at least two isolated nucleic acid molecules selected from the group consisting of (ao)-(bs); a bacterium comprising at least two isolated nucleic acid molecules selected from the group consisting of (a)-(an) and at least one isolated nucleic acid molecule selected from the group consisting of (ao)-(bs); a bacterium comprising at least two isolated nucleic acid molecules selected from the group consisting of (a)-(an) and at least two isolated nucleic acid molecules selected from the group consisting of (ao)-(bs); and a bacterium comprising an isolated nucleic acid molecule encoding a variant aspartokinase with reduced feedback inhibition, a variant homoserine dehydrogenase with reduced feedback inhibition, and/or a variant O-acetylhomoserine sulfhydrylase with reduced feedback inhibition.

Also described herein are methods for producing an amino acid or a related metabolite, comprising: cultivating (culturing) any of the forgoing bacterium under conditions that allow the amino acid or the related metabolite to be produced, and collecting a composition (culture medium, cells or a combination of cells and culture medium) that comprises the amino acid or related metabolite from the culture. The methods can further include: fractionating at least a portion of the culture to obtain a fraction that is enriched in the amino acid or the metabolite compared to culture that has not been fractionated.

Also described is a method for producing S-adenosylmethionine, the method comprising: cultivating a bacterium described herein under conditions that allow S-adenosylmethionine to be produced, and collecting a composition that comprises the S-adenosylmethionine from the culture. The method can include: fractionating at least a portion of the culture to obtain a fraction enriched in S-adenosylmethionine.

Also described is a method for producing methionine, the method comprising: cultivating a bacterium described herein under conditions that allow methionine to be produced, and collecting a composition that comprises the methionine from the culture. The method can include: fractionating at least a portion of the culture to obtain a fraction enriched in methionine.

Also described is a method for producing cysteine, the method comprising: cultivating a bacterium described herein under conditions that allow cysteine to be produced, and collecting a composition that comprises the cysteine from the culture. The method can include: fractionating at least a portion of the culture to obtain a fraction enriched in cysteine.

Also described is a method for producing lysine, the method comprising: cultivating a bacterium described herein under conditions that allow lysine to be produced, and collecting a composition that comprises the lysine from the culture. The method can include: fractionating at least a portion of the culture to obtain a fraction enriched in lysine.

Also described is a method for producing threonine or a related metabolite, the method comprising: cultivating a bacterium described herein under conditions that allow threonine or a related metabolite to be produced, and collecting a composition that comprises the threonine or a related metabolite from the culture. The method can include: fractionating at least a portion of the culture to obtain a fraction enriched in threonine or a related metabolite.

Also described is a method for producing isoleucine or a related metabolite, the method comprising: cultivating a bacterium described herein under conditions that allow isoleucine or a related metabolite to be produced, and collecting a composition that comprises the isoleucine or a related metabolite from the culture. The method can include: fractionating at least a portion of the culture to obtain a fraction enriched in isoleucine or a related metabolite.

Also described is a method for the preparation of animal feed additives containing one or more amino acids selected from the group consisting of methionine, S-adenosymethionine, cysteine, lysine, threonine, and isoleucine comprising: (a) cultivating a bacterium described herein under conditions that allow the selected amino acid(s) to be produced; (b) collecting a composition that comprises at least a portion of the selected amino acid(s) that result from cultivating the bacterium; (c) concentrating the collected composition to enrich the selected amino acid(s); and (d) optionally, adding one or more substances to obtain the desired feed (e.g., animal feed) additive. In various situations: the bacterium is an Escherichia coli or a coryneform bacterium; the bacterium is Corynebacterium glutamicum; the selected amino acid is methionine.

Also disclosed is a An Enterobacteriaceae or coryneform bacterium: comprising at least one isolated nucleic acid molecule selected from the group consisting of (a)-(an) and at least one isolated nucleic acid molecule selected from the group consisting of (ao)-(bs); comprising at least one isolated nucleic acid molecule selected from the group consisting of (a)-(an) and at least two isolated nucleic acid molecules selected from the group consisting of (ao)-(bs); comprising at least two isolated nucleic acid molecules selected from the group consisting of (a)-(an) and at least one isolated nucleic acid molecule selected from the group consisting of (ao)-(bs); comprising at least two isolated nucleic acid molecules selected from the group consisting of (a)-(an) and at least two isolated nucleic acid molecules selected from the group consisting of (ao)-(bs).

Also described are bacterium comprising: an isolated nucleic acid molecule encoding a variant aspartokinase with reduced feedback inhibition, a variant homoserine dehydrogenase with reduced feedback inhibition or a variant O-acetylhomoserine sulfhydrylase with reduced feedback inhibition (e.g., a bacterium wherein the variant aspartokinase with reduced feedback inhibition, the variant homoserine dehydrogenase with reduced feedback inhibition, or the variant O-acetylhomoserine sulfhydrylase with reduced feedback inhibition is heterologous to the host cell). Other examples include: a bacterium having a mutation in homoserine kinase that reduces or eliminates its expression or activity; a bacterium having a mutation in methionine/cysteine biosynthesis repression that reduces or eliminates its expression or activity (e.g., a bacterium having a mutation in the methionine and cysteine biosynthesis repressor (McbR)); a bacterium having a mutation in methionine adenosyltransferase that reduces its expression or activity; a bacterium that comprises (aj) and (ak); a bacterium that comprises (r), (s) and (t); and a bacterium that comprises (a), (b) and (c).

A “functional variant” protein is a protein that is capable of catalyzing the biosynthetic reaction catalyzed by the wild-type protein in the case where the protein is an enzyme, or providing the same biological function of the wild-type protein when that protein is not catalytic. For instance, a functional variant of a protein that normally regulates the transcription of one or more genes would still regulate the transcription of the same gene(s) when transformed into a bacterium. A functional variant can have the same level of activity as the wild-type protein or it can have increased or descreased activity. In certain embodiments, a functional variant protein is at least partially or entirely resistant to feedback inhibition by a product or an intermediate of an amino acid biosynthetic pathway. In certain embodiments, the variant has fewer than 20, 15, 10, 9, 8, 7, 6, 5, 4, 3, or 2 amino acid changes compared to the wild-type protein. In certain embodiments, the amino acid changes are conservative changes. A variant sequence is a nucleotide or amino acid sequence corresponding to a variant polypeptide, e.g., a functional variant polypeptide.

An amino acid that is “corresponding” to an amino acid in a reference sequence occupies a site that is homologous to the site in the reference sequence. Corresponding amino acids can be identified by alignment of related sequences. Amino acid sequences can be compared to protein sequences available in public databases using algorithms such as BLAST, FASTA, ClustalW, which are well known to those skilled in the art.

As used herein, a “heterologous” nucleic acid or protein is meant to encompass a nucleic acid or protein, or functional variant of a nucleic acid or protein, of an organism (species) other than the host organism (species) used for the production of members of the aspartic acid family of amino acids and related metabolites. In certain embodiments, when the host organism is a coryneform bacteria the heterologous gene will not be obtained from E. coli. In other embodiments, when the host organism is E. coli the heterologous gene will not be obtained from a coryneform bacteria.

“Gene”, as used herein, includes coding, promoter, operator, enhancer, terminator, co-transcribed (e.g., sequences from an operon), and other regulatory sequences associated with a particular coding sequence.

As used herein, a “homologous” nucleic acid or protein is meant to encompass a nucleic acid or protein, or functional variant of a nucleic acid or protein, of an organism that is the same species as the host organism used for the production of members of the aspartic acid family of amino acids and related metabolites.

A “recombinant nucleic acid molecule” is a nucleic acid molecule that is not present in its natural context. For example, a nucleic acid molecule which exactly encodes an E. coli polypeptide is recombinant when it is inserted into the E. coli genome at a location that is other than the wild-type location for the gene encoding the polypeptide. A recombinant nucleic acid molecule also includes a nucleic acid molecule consisting of a non-wild type promoter and a wild-type polypeptide coding sequence inserted into the genome of a bacterium at either the wild-type location of the gene encoding the polypeptide or at some other location.

As known to those skilled in the art, certain substitutions of one amino acid for another may be tolerated at one or more amino acid residues of a wild-type enzyme without eliminating the activity or function of the enzyme. As used herein, the term “conservative substitution” refers to the exchange of one amino acid for another in the same conservative substitution grouping in a protein sequence. Conservative amino acid substitutions are known in the art and are generally based on the relative similarity of the amino acid side-chain substituents, for example, their hydrophobicity, hydrophilicity, charge, size, and the like. In one embodiment, conservative substitutions typically include substitutions within the following groups: Group 1: glycine, alanine, and proline; Group 2: valine, isoleucine, leucine, and methionine; Group 3: aspartic acid, glutamic acid, asparagine, glutamine; Group 4: serine, threonine, and cysteine; Group 5: lysine, arginine, and histidine; Group 6: phenylalanine, tyrosine, and tryptophan. Each group provides a listing of amino acids that may be substituted in a protein sequence for any one of the other amino acids in that particular group.

There are several criteria used to establish groupings of amino acids for conservative substitution. For example, the importance of the hydropathic amino acid index in conferring interactive biological function on a protein is generally understood in the art (Kyte and Doolittle, Mol. Biol. 157:105-132 (1982). It is known that certain amino acids may be substituted for other amino acids having a similar hydropathic index or score and still retain a similar biological activity. Amino acid hydrophilicity is also used as a criterion for the establishment of conservative amino acid groupings (see, e.g., U.S. Pat. No. 4,554,101).

Information relating to the substitution of one amino acid for another is generally known in the art (see, e.g., Introduction to Protein Architecture: The Structural Biology of Proteins, Lesk, A. M., Oxford University Press; ISBN: 0198504748; Introduction to Protein Structure, Branden, C.-I., Tooze, J., Karolinska Institute, Stockholm, Sweden (Jan. 15, 1999); and Protein Structure Prediction: Methods and Protocols (Methods in Molecular Biology), Webster, D.M. (Editor), August 2000, Humana Press, ISBN: 0896036375).

In some embodiments, the nucleic acid and/or protein sequences of a heterologous sequence and/or host strain gene will be compared, and the homology can be determined. Homology comparisons can be used, for example, to identify corresponding amino acids. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. For example, the percent identity between two nucleotide sequences can be determined using the algorithm of Needleman and Wunsch ((1970) J. Mol Biol. 48:444-453) algorithm which has been incorporated into the GAP program in the GCG software package, using either a Blosum 62 matrix and a gap weight of 12, a gap extend penalty of 4, and a frameshift gap penalty of 5.

Generally, to determine the percent identity of two nucleic acid or protein sequences, the sequences are aligned for optimal comparison purposes (e.g., gaps can be introduced in one or both of a first and a second nucleic acid or amino acid sequence for optimal alignment and non-homologous sequences can be disregarded for comparison purposes). The length of a test sequence aligned for comparison purposes can be at least 30%, 40%, 50%, 60%, 70%, 80%, 90%, 100% of the length of the reference sequence. The nucleotides or amino acids at corresponding nucleotide or amino acid positions are then compared. When a position in the first sequence is occupied by the same nucleotide or amino acid as the corresponding position in the second sequence, then the molecules are identical at that position (as used herein “identity” is equivalent to “homology”).

The protein sequences described herein can be used as a “query sequence” to perform a search against a database of non-redundant sequences, for example. Such searches can be performed using the BLASTP and TBLASTN programs (version 2.0) of Altschul, et al. (1990) J. Mol Biol. 215:403-10. BLAST protein searches can be performed with the BLASTP program, using, for example, the Blosum 62 matrix, a wordlength of 3, and a gap existence cost of 11 and a gap extension penalty of 1. Software for performing BLAST analyses is publicly available through the National Center for Biotechnology Information, and default paramenter can be used. Sequences described herein can also be used as query sequences in TBLASTN searches, using specific or default parameters.

The nucleic acid sequences described herein can be used as a “query sequence” to perform a search against a database of non-redundant sequences, for example. Such searches can be performed using the BLASTN and BLASTX programs (version 2.0) of Altschul, et al. (1990) J. Mol Biol. 215:403-10. BLAST nucleotide searches can be performed with the BLASTN program, score=100, wordlength=11 to evaluate identity at the nucleic acid level. BLAST protein searches can be performed with the BLASTX program, score=50, wordlength=3 to evaluate identity at the protein level. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., BLASTX and BLASTN) can be used. Alignment of nucleotide sequences for comparison can also be conducted, e.g., by the local homology algorithm of Smith & Waterman, Adv. Appl. Math. 2:482 (1981), by the homology alignment algorithm of Needleman & Wunsch, J. Mol Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Nucleic acid sequences can be analyzed for hybridization properties. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1 % SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one, two, three, four or more washes in 0.2×SSC, 0.1% SDS at 65° C.) very high stringency hybridization conditions are 0.5M sodium phosphate, 7% SDS at 65° C., followed by one or more washes at 0.2×SSC, 1% SDS at 65° C. Very high stringency conditions (at least 4 or more washes) are the preferred conditions and the ones that should be used unless otherwise specified.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of the methionine biosynthetic pathway in bacteria.

FIG. 2 is a diagram of the cysteine and serine biosynthetic pathway in bacteria.

FIG. 3 is a diagram of the sulfate assimilation pathway in bacteria.

FIG. 4a is a diagram of the folate biosynthetic pathway in bacteria.

FIG. 4b is a diagram of the glycine cleavage system in bacteria

FIG. 5 is a restriction map of plasmid MB3961 (vector backbone plasmid).

FIG. 6 is a restriction map of plasmid MB4094 (vector backbone plasmid).

FIG. 7 is a restriction map of plasmid MB4083 (hom-thrB deletion construct).

FIG. 8 is a restriction map of plasmid MB4084 (thrB deletion construct).

FIG. 9 is a restriction map of plasmid MB4165 (mcbR deletion construct).

FIG. 10 is a restriction map of plasmid MB4169 (hom-thrB deletion/gpd-M. smegmatis lysC(T311I)-asd replacement construct).

FIG. 11 is a restriction map of plasmid MB4192 (hom-thrB deletion/gpd-S. coelicolor hom(G362E) replacement construct.

FIG. 12 is a restriction map of plasmid MB4276 (pck deletion/gpd-M. smegmatis lysC(T311I)-asd replacement construct).

FIG. 13 is a restriction map of plasmid MB4286 (mcbR deletion/trcRBS-T. fusca metA replacement construct).

FIG. 14A is a restriction map of plasmid MB4287 (mcbR deletion/trcRBS-C. glutamicum metA (K233A)-metB replacement construct).

FIG. 14B is a depiction of the nucleotide sequence of the DNA sequence in MB4278 (trcRBS-C. glutamicum metAYH) that spans from the trcRBS promoter to the stop of the metH gene.

FIG. 15 is a graph depicting the results of an assay to determine in vitro O-acetyltransferase activity of C. glutamicum MetA from two C. glutamicum strains, MA-442 and MA-449, in the presence and absence of IPTG.

FIG. 16 is a graph depicting the results of an assay to determine sensitivity of MetA in C. glutamicum strain MA-442 to inhibition by methionine and S-AM.

FIG. 17 is a graph depicting the results of an assay to determine the in vitro O-acetyltransferase activity of T. fusca MetA expressed in C. glutamicum strains MA-456, MA570, MA-578, and MA-479. Rate is a measure of the change in OD412 divided by time per nanograms of protein.

FIG. 18 is a graph depicting the results of an assay to determine in vitro MetY activity of T. fusca MetY expressed in C. glutamicum strains MA-456 and MA-570. Rate is defined as the change in OD412 divided by time per nanograms of protein.

FIG. 19 is a graph depicting the results of an assay to determine lysine production in C. glutamicum and B. lactofermentum strains expressing heterologous wild-type and mutant lysC variants.

FIG. 20 is a graph depicting results from an assay to determine lysine and homoserine production in C. glutamicum strain, MA-0331 in the presence and absence of the S. coelicolor hom G362E variant.

FIG. 21 is a graph depicting results from any assay to determine asparate concentrations in C. glutamicum strains MA-0331 and MA-0463 in the presence and absence of E chrysanthemi ppc.

FIG. 22 is a graph depicting results from an assay to determine lysine production in C. glutamicum strains MA-0331 and MA-0463 transformed with heterologous wild-type dapA genes.

FIG. 23 is a graph depicting results from an assay to determine metabolite levels in C. glutamicum strain MA-1378 and its parent strains.

FIG. 24 is a graph depicting results from an assay to determine homoserine and O-acetylhomoserine levels in C. glutamicum strains MA-0428, MA-0579, MA-1351, MA-1559 grown in the presence or absence of IPTG. IPTG induces expression of the episomal plasmid borne T. fusca metA gene.

FIG. 25 is a graph depicting results from an assay to determine metabolite levels in C. glutamicum strain MA-1559 and its parent strains.

FIG. 26 is a graph depicting methionine concentrations in broths from fermentations of two C. glutamicum strains, MA-622, and MA-699, which express a MetA K233A mutant polypeptide. Production by cells cultured in the presence and absence of IPTG is depicted.

FIG. 27 is a graph depicting methionine concentrations in broths from fermentations of two C. glutamicum strains, MA-622 and MA-699, expressing a MetY D231 A mutant polypeptide. Production by cells cultured in the presence and absence of IPTG is depicted.

FIG. 28 is a graph depicting methionine concentrations in broths from fermentations of two C. glutamicum strains, MA-622 and MA-699, expressing a C. glutamicum MetY G232A mutant polypeptide. Production by cells cultured in the presence and absence of IPTG is depicted.

FIG. 29 is a graph depicting results from an assay to determine metabolite levels in C. glutamicum strains MA-1906, MA-2028, MA-1907, and MA-2025. Strains were grown in the presence and absence of IPTG.

FIG. 30 is a graph depicting results from an assay to determine metabolite levels in C. glutamicum strains MA-1667 and MA-1743. Strains were grown in the presence and absence of IPTG.

FIG. 31 is a graph depicting results from an assay to determine metabolite levels in C. glutamicum strains MA-0569, MA-1688, MA-1421, and MA-1790. Strains were grown in the absence and/or presence of IPTG.

FIG. 32 is a graph depicting results from an assay to determine metabolite levels in C. glutamicum strain MA-1668 and its parent strains.

FIG. 33 is a table providing the sequences of certain useful polypeptides and nucleic acid molecules.

FIG. 34 is a table providing the sequences of certain additional useful polypeptides and nucleic acid molecules.

### DETAILED DESCRIPTION

Genetically modified bacteria that harbor nucleic acid sequences encoding proteins that improve fermentative production of methionine and methionine-related intermediate compounds and other amino acids and metabolites are described herein. In particular, nucleic acid molecules, polypeptides and bacteria relevant to the production of methionine, S-adenosyl-methionine, homoserine, O-acetyl homoserine, homocysteine, and cystathionine and other compounds are described. The nucleic acids encode metabolic pathway proteins that modulate the biosynthesis of these amino acids, intermediates, and related metabolites either directly (e.g., via enzymatic conversion of intermediates) or indirectly (e.g., via transcriptional regulation of enzyme expression, regulation of amino acid export, or regulation of metabolite uptake). The nucleic acid sequences encoding the proteins can be derived from bacterial species other than the host organism and such sequences and proteins are referred to as heterologous to the host. Other nucleic acids and encoded proteins are derived from the same species as the host organism and such sequences and proteins are referred to as homologous to the host. In some circumstances a host organism is genetically modified to contain both homologous and heterologous nucleic acid sequences. Methods for producing genetically modified bacteria are described as are methods for producing amino acids and metabolites, including method for the production of amino acids for use in animal feed additives. The introduction of a nucleic acid sequence encoding a heterologous or homologous polypeptide can lead to increased yields of one or more amino acids and/or intermediates. In addition, modification of the sequences of certain bacterial proteins involved in amino acid production can lead to increased yields of amino acids and/or intermediates. For example, a mutation in a coding sequence for a polypeptide can lead to decreased or increased activity of a polypeptide (e.g, decreased or increased enzymatic activity).

Regulated (e.g., reduced or increased) expression of modified or unmodified (e.g., wild type) bacterial proteins can likewise enhance amino acid production. The methods and compositions described herein apply to bacterial proteins that regulate the production of amino acids and related metabolites, (e.g., proteins involved in the metabolism or export of methionine, serine, homoserine, cysteine, cystathionine, folate, vitamin B12, homocysteine, and sulfur), and nucleic acids encoding these proteins. These proteins include enzymes that catalyze the conversion of intermediates of amino acid biosynthetic pathways to other intermediates and/or end products, proteins that directly regulate the expression and/or function of such enzymes, and proteins that regulate the uptake of metabolites utilized in the biosynthetic pathways. Target proteins for manipulation include those enzymes that are subject to various types of regulation such as repression, attenuation, or feedback-inhibition. Information regarding amino acid biosynthetic pathways in bacterial species, the proteins involved in these pathways, links to sequences of these proteins, and other related resources for identifying proteins for manipulation and/or expression as described herein are described in Bono et al., Genome Research, 8:203-210, 1998. Strategies to manipulate the efficiency of amino acid biosynthesis for commercial production include, but are not limited to, overexpression (e.g., due to increased gene dosage, modification of (including replacement of) expression control sequences or alterations in regulatory proteins), underexpression (e.g., due to gene disruption or replacement or the use of anti-sense technologies), and conditional expression of specific genes, as well as genetic modification to optimize the activity of proteins. Underexpression or reduced activity of a selected polypeptide can arise from producing less mRNA encoding the selected polypeptide (reduced transcription), producing less polypeptide, even where mRNA production is not reduced (e.g., reduced translation) or from altering the sequence encoding the polypeptide so that inactive or less active polypeptide is produced.

It is possible to reduce the sensitivity of polypeptides to inhibitory stimuli, e.g., feedback inhibition due to the presence of biosynthetic pathway end products and intermediates. For example, strains used for commercial production of lysine derived from either coryneform bacteria or Escherichia coli typically display relative insensitivity to feedback inhibition by lysine. Useful coryneform bacterial strains are also relatively resistant to inhibition by threonine. Novel methods and compositions described herein result in enhanced amino acid production.

Biosynthesis of Methionine

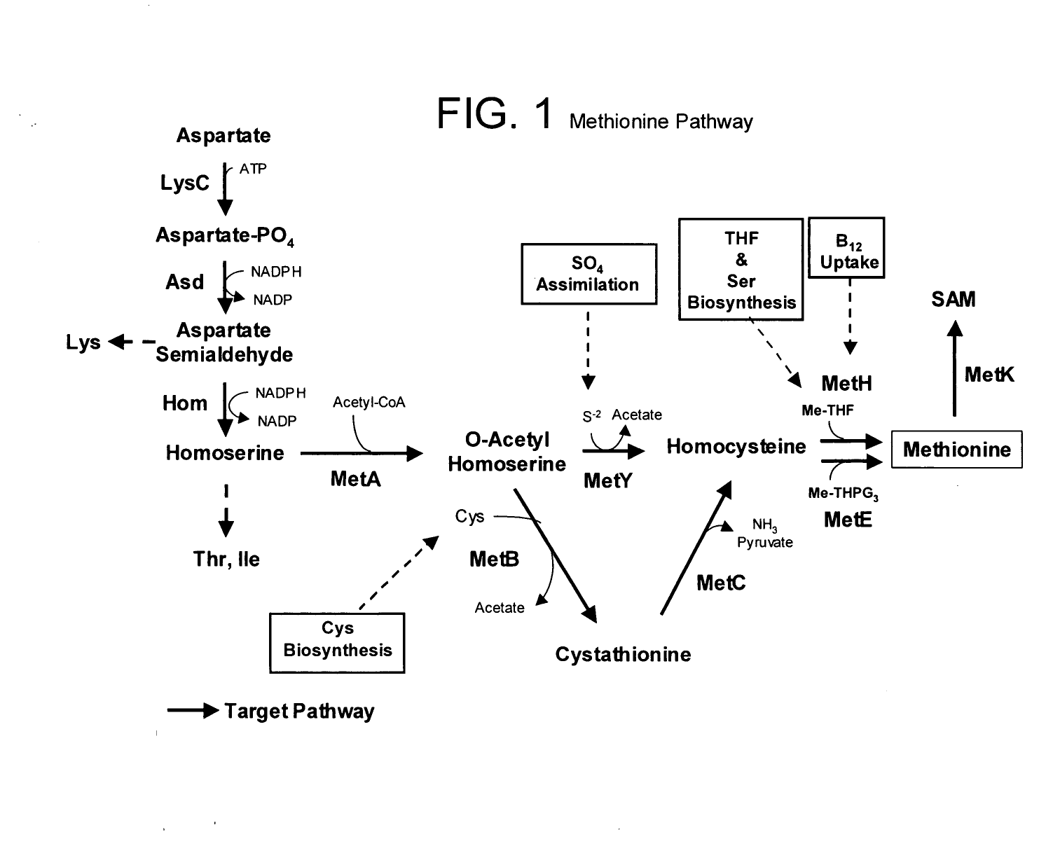

The biosynthesis of methionine and other aspartic acid family amino acids (and intermediates) starting from the conversion of aspartate is diagrammed in FIG. 1. A list of enzymes in the methionine biosynthesis pathway is provided in Table 1. Overexpression and/or deregulation of each of these enzymes can enhance production of methionine. Overexpression of biosynthetic enzymes can be achieved, for example, by increasing copy number of the gene of interest and/or operably linking the gene to a promoter optimal for expression, e.g., a strong or conditional promoter.

TABLE 1
Genes and Enzymes in the Methionine Biosynthesis Pathway

| Gene | Enzyme | Step |
|---|---|---|
| lysC | Aspartokinase (EC 2.7.2.4) | Converts Aspartate to Aspartate Phosphate |
| asd | Aspartic | Converts Aspartate Phosphate to Converts |
| | semialdehydedehydrogenase (EC | Aspartate Semialdehyde |
| | 1.2.1.11) | |
| hom | Homoserine dehydrogenase (EC | Converts Aspartate Semialdehyde to |
| | 1.1.1.3) | Homoserine |
| metA | O-homoserine acetyltransferase | Converts Homoserine to O-Acetyl |
| | (EC 2.3.1.31) | Homoserine |
| metY | O-acetylhomoserine | Converts O-Acetyl Homoserine to |
| | sulfhydrylase (EC 2.5.1.49) | Homocysteine. |
| metH | Cobalamin-Dependent | Cobalamin dependent conversion of |
| | Methionine Synthase (EC | Homocysteine to Methionine |
| | 2.1.1.13) | |
| metB | O-succinyl(acetyl)homoserine | Converts O-Acetyl Homoserine to Cystathionine |
| | (thio)-lyase (cystathionine | |
| | gamma-lyase) (EC 2.5.1.48) | |
| metC | Cystathionine beta-lyase (EC | Converts Cystathionine to Homocysteine |
| | 4.4.1.8) | |
| metE | Cobalamin-Independent | Cobalamin independent conversion of Homocysteine |
| | Methionine Synthase (EC | to Methionine |
| | 2.1.1.14) | |

Methionine Biosynthesis Precursors and Cofactors

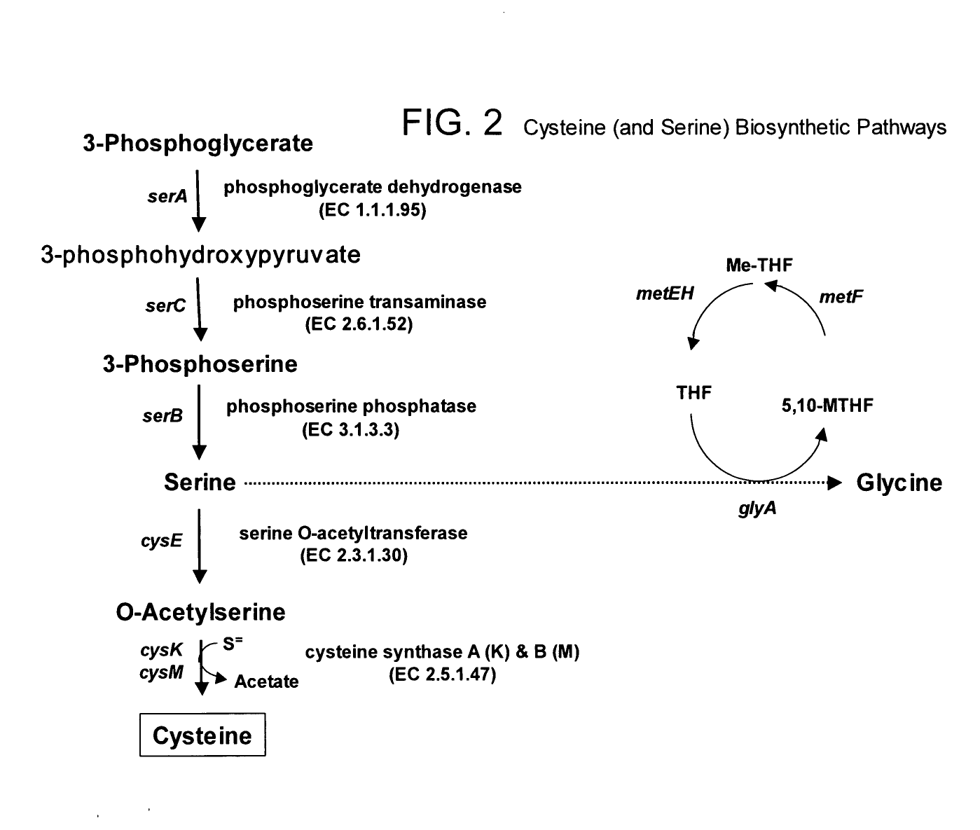

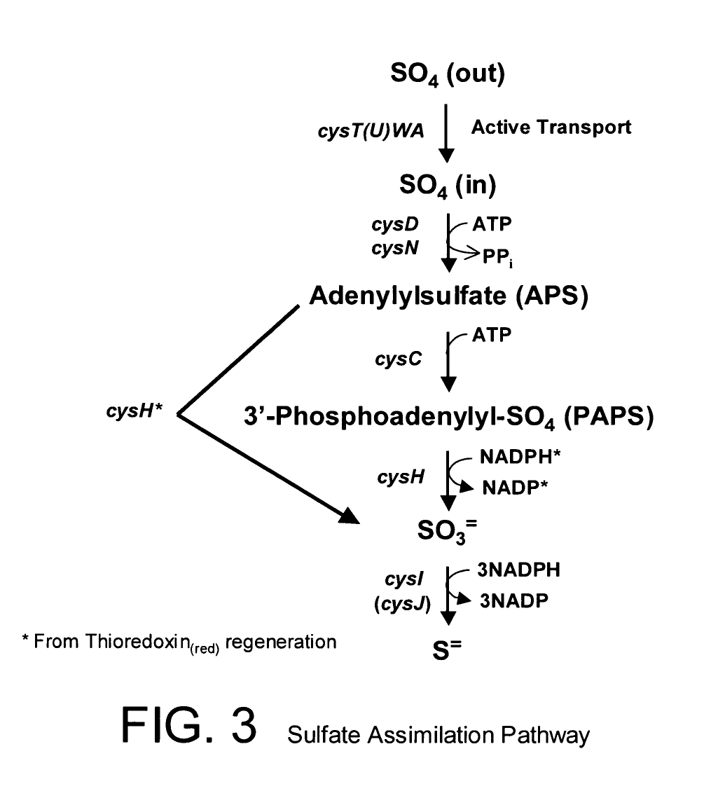

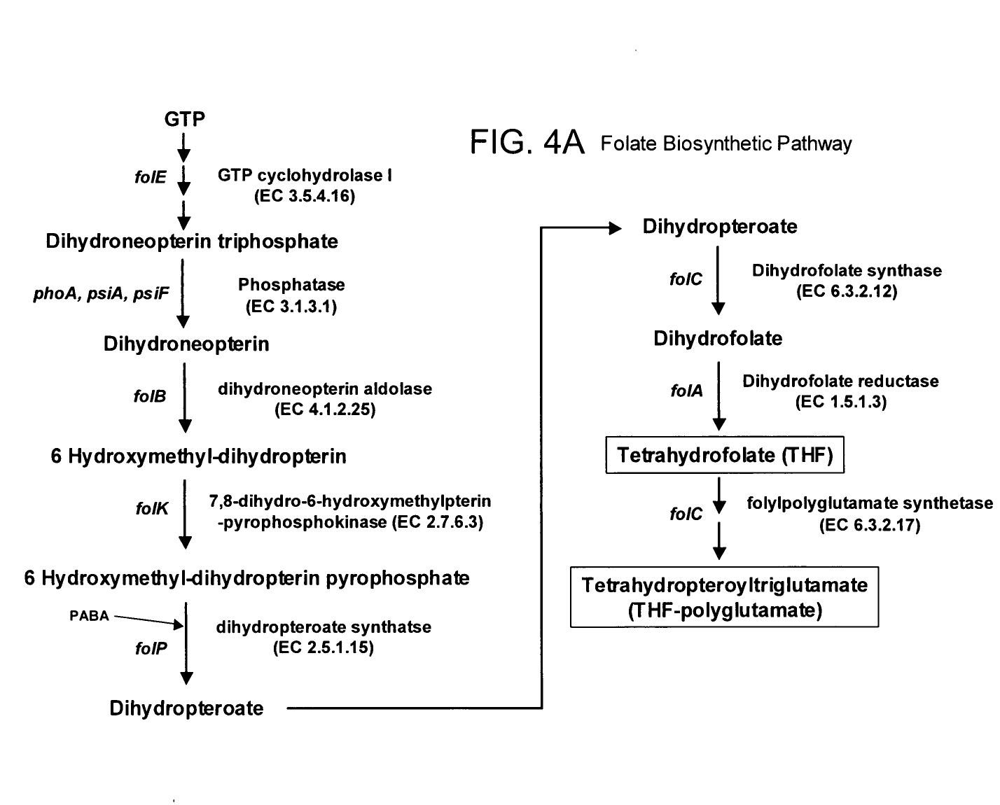

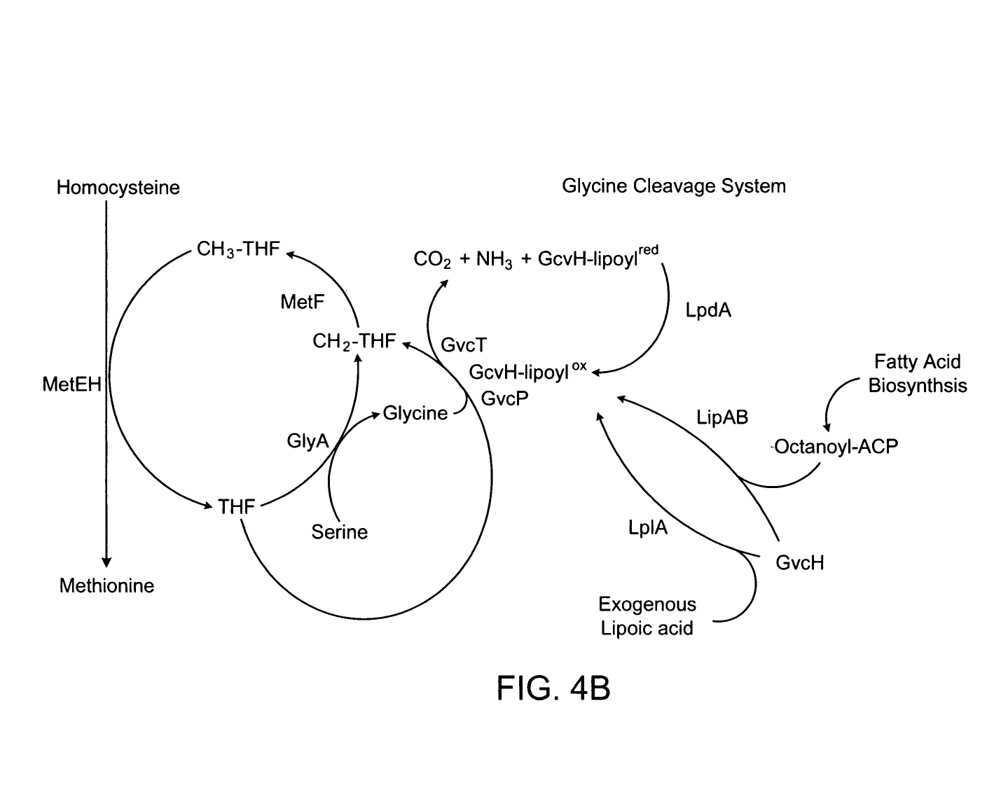

The biochemical pathways that yield the precursors and cofactors used in the methionine pathway are also important for determining the level of methionine production, as illustrated in FIG. 1. Precursor pathways include, for example, serine and cysteine biosynthesis (FIG. 2), sulfate assimilation (FIG. 3), folate biosynthesis (FIG. 4), and vitamin B12 uptake.

Serine and Cysteine Biosynthesis

Cysteine is a co-factor in the conversion of O-succinyl homoserine or O-acetyl homoserine to cystathionine by cystathionine gamma-synthase (MetB), as shown in FIG. 1. Table 2 lists the proteins that act in the pathway in which D-3-phosphoglycerate is converted to cysteine and the reactions they catalyze (see FIG. 2).

TABLE 2
Conversion of D-3-Phosphoglycerate to Cysteine

| Gene | Protein | Step |
|---|---|---|
| serA | D-3-Phosphoglycerate | D-3-Phosphoglycerate to |
| | dehydrogenase (EC 1.1.1.95) | D-3-Phosphohydroxypyruvate |
| serC | D-3-Phosphoserine | D-3-Phosphohydroxypurvate to |
| | transaminase | D-3-Phosphoserine |
| | (EC 2.6.1.52) | |
| serB | D-3-Phosphoserine | D-3-Phosphoserine to Serine |
| | phosphatase | |
| | (EC 3.1.3.3) | |
| cysE | Serine-O-acetyltransferase | Serine to |
| | (EC 2.3.1.30) | O-Acetylserine |
| cysK | Cysteine Synthase A & B | O-Acetylserine to |
| cysM | (EC 2.5.1.47) | Cysteine |

Phosphoglycerate Dehydrogenase

Phosphoglycerate dehydrogenase (SerA) converts 3-phosphoglycerate to 3-phosphohydroxypyruvate, a precursor in the cysteine biosynthesis pathway. Cysteine can be converted to cystathionine, which is a precursor to methionine. Thus, increased SerA expression or activity can increase methionine or S-adenosyl L-methionine production. In addition, phosphohydroxypyruvate is a precursor of serine, which is required to regenerate methyltetrahydrofolate, which is required to convert homocysteine to methionine. Thus, increased SerA expression or activity may increase methionine production by generating methyltetrahydrofolate.

Phosphoserine Transaminase

Phosphoserine transaminase (SerC) converts phosphohydroxypyruvate to 3-phosphoserine, a precursor in the cysteine biosynthesis pathway. Cysteine can be converted to cystathionine, which is a precursor to methionine. Thus, increased SerC expression or activity can increase methionine or S-adenosyl L-methionine production. In addition, phosphohydroxypyruvate is a precursor of serine, which is required to regenerate methyltetrahydrofolate, which is required to convert homocysteine to methionine. Thus, increased SerC expression or activity may increase methionine or S-adenosyl L-methionine production by generating methyltetrahydrofolate.

Phosphoserine Phosphatase

Phosphoserine phosphatase (SerB) converts phosphoserine to the amino acid serine, a precursor in the cysteine biosynthesis pathway. Cysteine can be converted to cystathionine, which is a precursor to methionine. Thus, increased SerB expression or activity can increase methionine or S-adenosyl L-methionine production. In addition, phosphohydroxypyruvate is a precursor of serine, which is required to regenerate methyltetrahydrofolate, which is required to convert homocysteine to methionine. Thus, increased SerB expression or activity may increase methionine or S-adenosyl L-methionine production by generating methyltetrahydrofolate.

Serine O-Acetyltransferase

Serine O-acetyltransferase (CysE) catalyzes the conversion of serine into O-acetylserine, a precursor in the cysteine biosynthesis pathway. Cysteine can be converted to cystathionine, which is a precursor to methionine. Thus, increased CysE expression or activity can increase methionine or S-adenosyl L-methionine production.

Cysteine Synthase A and Cysteine Synthase B

Cysteine synthase A (CysK) and cysteine synthase B (CysM) catalyze the 5 conversion of O-acetylserine into cysteine. Cysteine can be converted to cystathionine which is a precursor to methionine. Thus, increased CysK and/or CysM expression or activity can increase methionine or S-adenosyl L-methionine production.

Sulfate Assimilation

Sulfate (SO₄) assimilation is important to the production of sulfide (S²⁻) which acts as an oxiding agent in the conversion of O-Acetyl homoserine to Homocysteine (See FIG. 1). Table 3 lists proteins that function in SO₄ assimilation and the conversion steps to sulfide (see FIG. 3).

TABLE 3
Assimilation of SO₄ and its Conversion to S⁻²

| Gene | Protein | Step |
|---|---|---|
| cysA | Sulfate ABC transporter ATP- | Transport of extracellular SO₄ |
| | binding protein (permease A | |
| | protein; EC 3.6.3.25); | |
| cysW | Sulfate transport system | |
| | permease W protein; and | |
| cysT | Sulfate, thiosulfate transport | |
| | system permease T protein | |
| cysN | Sulfate adenylyltransferase | Conversion of SO₄ to |
| | subunit 1 (EC 2.7.7.4); | Adenylylsulfate |
| cysD | Sulfate adenylyltransferase | |
| | subunit 2 (EC 2.7.7.4) | |
| cysC | Adenylylsulfate kinase (EC | Conversion of |
| | 2.7.1.25) | Adenylylsulfate to 3′- |
| | | Phosphoadenylyl-SO₄ (PAPS) |
| cysH | Adenylylsulfate reductase, (EC | Conversion of |
| | 1.8.99.2) | Adenylylsulfate to SO₃²⁻ |
| cysH | Phosphoadenosine | Conversion of 3′- |
| | phosphosulfate reductase (EC | Phosphoadenylyl-SO4 |
| | 1.8.4.8) | (PAPS) to SO₃²⁻ |
| cysI | Sulfite reductase alpha subunit | Conversion of SO₃²⁻ to S²⁻ |
| | or hemoprotein beta-component | |
| | (EC 1.8.1.2); | |
| cysJ | Sulfite reductase (NADPH), | |
| | flavoprotein beta subunit (EC | |
| | 1.8.1.2) | |

Sulfate Assimilation
ABC transporter ATP-binding protein (permease A protein)
Sulfate transport system permease W protein
Sulfate, thiosulfate transport system permease T protein

Sulfate ABC transporter ATP-binding protein (CysA), sulfate transport system permease W protein (CysW), and sulfate, thiosulfate transport system permease T protein (CysT) function in the transport of extracellular SO₄ into the cell. SO₄ is a precursor to S²⁻, which serves as an oxidizing agent for the conversion of O-acetylhomoserine to homocysteine by MetY. Increasing production of homocysteine can lead to increased production of methionine. Thus, increased CysA, CysW, and/or CysT expression or activity can increase methionine or S-adenosyl-L-methionine production.

Sulfate Adenylyltransferase Subunit 1 and 2

Sulfate adenylyltransferase subunit 1 (CysN) and sulfate adenylyltransferase subunit 2 (CysD) convert SO₄ to adenylylsulfate, which serves as a precursor in S²⁻ production. S²⁻ serves as an oxidizing agent for the conversion of O-acetylhomoserine to homocysteine by MetY. Increasing production of homocysteine can lead to increased production of methionine. Thus, increased CysN and/or CysD expression or activity can increase methionine or S-adenosyl-L-methionine production.

Adenylylsulfate Kinase

Adenylsulfate kinase (CysC) phosphorylates adenylylsulfate thereby converting it to 3′-phosphoadenylyl-sulfate, which serves as a precursor to the production of S²⁻ which serves as an oxidizing agent for the conversion of O-acetylhomoserine to homocysteine by MetY. Thus, increased CysC expression or activity can increase methionine or S-adenosyl-L-methionine production.

Adenylylsulfate Reductase (Assimilatory-Type)

Adenylylsulfate reductase (CysH) serves to produce SO₃²⁻ from the reduction of adenylylsulfate. SO₃²⁻ serves as a precursor for S²⁻ formation, and S²⁻ serves as an oxidizing agent for the conversion of O-acetylhomoserine to homocysteine by MetY. Thus, increased CysH expression or activity can increase methionine or S-adenosyl-L-methionine production.

Phosphoadenosine Phosphosulfate Reductase

Phosphoadenosine phosphosulfate reductase (CysH) activity serves to produce SO₃²⁻ from the reduction of 3′-phosphoadenylyl-sulfate by NADPH. SO₃²⁻ serves as a precursor for S²⁻ formation, and S²⁻ is an oxidizing agent for the conversion of O-acetylhomoserine to homocysteine by MetY. Thus, increased CysH expression or activity can increase methionine or S-adenosyl-L-methionine production.

Sulfite Reductase (Alpha Subunit or Hemoprotein Beta-Component, CysI) and Sulfite Reductase (NADPH), Flavoprotein Beta Subunit, CysJ)

The sulfite reductases CysI and CysJ convert SO₃²⁻ to S²⁻ which serves as an oxidizing agent for the conversion of O-acetylhomoserineto homocysteine by MetY. Thus, increased CysI and/or CysJ expression or activity can increase methionine or S-adenosyl-L-methionine production.

Folate Biosynthesis

In enterobacteria, 5-methyltetrahydrofolate, which is produced in the folate biosynthetic pathway, acts as a methyl group donor to homocysteine thereby converting it to methionine (see FIG. 1). Table 4 lists proteins that function in the folate biosynthetic pathway (see FIG. 4).

TABLE 4
Folate Biosynthetic Pathway

| Gene | Protein | Step |
|---|---|---|
| folE | GTP cyclohydrolase I (EC | Conversion of GTP to |
| | 3.5.4.16) | Dihydroneopterin triphosphate |
| phoA (also | Phosphatase (EC 3.6.1.) | Conversion of Dihydroneopterin |
| psiA and psiF) | | triphosphate to Dihydroneopterin |
| Folb (also | Dihydroneopterin aldolase (EC | Conversion of Dihydroneopterin to 3′-6 |
| ygiG) | 4.1.2.25) | Hydroxymethyl-dihydropterin |
| folK | 7,8-dihydro-6- | Conversion of 3′-6 |
| | hydroxymethylpterin- | Hydroxymethyl-dihydropterin to 6 |
| | pyrophosphokinase (EC 2.7.6.3) | Hydroxymethyl-dihydropterin |
| | | pyrophosphate |
| folP (also | Dihydropteroate synthase (FolP, | Conversion of 6 Hydroxymethyl- |
| dphS) | DhpS; | dihydropterin pyrophosphate to |
| | EC 2.5.1.15) | Dihydropteroate |
| | Dihydrofolate synthetase (FolC, | Conversion of Dihydropteroate to |
| | DedC; | Dihydrofolate |
| | EC 6.3.2.12) | |
| folA (also | Dihydrofolate reductase (FolA, | Conversion of Dihydrofolate to |
| tmrA) | TmrAEC 1.5.1.3) | Tetrahydrofolate |
| FolC | Folylpolyglutamate synthetase | Conversion of Tetrahydrofolate to |
| | (Dihydrofolate synthetaseEC | Tetrahydropteroyltriglutamate |
| | 6.3.2.17) | |

Folate Biosynthesis
GTP Cyclohydrolase I

GTP cyclohydrolase I (FolE) catalyzes the conversion of GTP to dihydroneopterin triphosphate a precursor in the biosynthesis of tetrahydrofolate (THF) and tetrahydropteroyltriglutamate (THFPG₃). THF and THFPG₃ are essential cofactors in the conversion of homocysteine to methionine by MetH or MetE, respectively. Thus, increased FolE expression or activity can increase methionine or S-adenosyl L-methionine production.

Phosphatase (PhoA, PsiA, PsiF)

Phosphatase(s) (PhoA, PsiA, PsiF) convert dihydroneopterin triphosphate to dihydroneopterin, a precursor in the biosynthesis of tetrahydrofolate (THF) and tetrahydropteroyltriglutamate (THFPG₃). THF and THFPG3 are essential cofactors in the conversion of homocysteine to methionine by MetH or MetE, respectively. Thus, increased PhoA, PsiA, and/or PsiF expression or activity can increase methionine or S-adenosyl L-methionine production.

Dihydroneopterin Aldolase

Dihydroneopterin aldolase (FolB) catalyzes the conversion of dihydroneopterin to 6-hydroxymethyl-dihydropterin, a precursor in the biosynthesis of tetrahydrofolate (THF) and tetrahydropteroyltriglutamate (THFPG₃). THF and THFPG₃ are essential cofactors in the conversion of homocysteine to methionine by MetH or MetE, respectively. Thus, increased FolB expression or activity can increase methionine or S-adenosyl L-methionine production.

7,8-dihydro-6-hydroxymethylpterin-pyrophosphokinase

7,8-dihydro-6-hydroxymethylpterin-pyrophosphokinase (FolK) catalyzes the conversion of 6-hydroxymethyl-dihydropterin to 6-hydroxymethyl-dihyropterin pyrophosphate, a precursor in the biosynthesis of tetrahydrofolate (THF) and tetrahydropteroyltriglutamate (THFPG₃). THF and THFPG₃ are essential cofactors in the conversion of homocysteine to methionine by MetH or MetE, respectively. Thus, increased FolK expression or activity can increase methionine or S-adenosyl L-methionine production.

Dihydropteroate Synthase

Dihydropteroate synthase (FOlP) converts 6-hydroxymethyl-dihyropterin pyrophosphate to dihydropteroate, a precursor in the biosynthesis of tetrahydrofolate (THF) and tetrahydropteroyltriglutamate (THFPG₃). THF and THFPG₃ are essential cofactors in the conversion of homocysteine to methionine by MetH or MetE, respectively. Thus, increased FolP expression or activity can increase methionine or S-adenosyl L-methionine production.

Dihydrofolate Synthase

Dihydrofolate synthase (FolC) catalyzes the conversion of dihydropteroate to dihydrofolate, a precursor in the biosynthesis of tetrahydrofolate (THF) and tetrahydropteroyltriglutamate (THFPG₃). THF and THFPG₃ are essential cofactors in the conversion of homocysteine to methionine by MetH or MetE, respectively. Thus, increased FoIC expression or activity can increase methionine or S-adenosyl L-methionine production.

Dihydrofolate Reductase

Dihydrofolate reductase (FolA) catalyzes the conversion of dihydrofolate to tetrahydrofolate (THF), a precursor to THFPG₃. THF and THFPG₃ are essential cofactors in the conversion of homocysteine to methionine by MetH or MetE, respectively. Thus, increased FolA expression or activity can increase methionine or S-adenosyl L-methionine production.

Folylpolyglutamate Synthetase

Folylpolyglutamate synthetase (FolC), which is also a dihydrofolate synthase (as described above), catalyzes the conversion of tetrahydrofolate to tetrahydropteroyltriglutamate (THFPG₃), which is an essential cofactor in the conversion of homocysteine to methionine by MetE. Thus, increased FolC expression or activity can increase methionine or S-adenosyl L-methionine production.

B12 Uptake & Metabolism

Vitamin B12 (cyanocobalamin) serves as a precursor to methylcobalamin, which is a cofactor required by MetH for the conversion of homocysteine to methionine. Proteins in the B12 uptake pathway include the btu genes listed in Table 5a. PduO catalyzes an adenosyltransferase reaction that yields adenosylcobalamin, which is required by some other vitamin B12-dependent enzymes, but not MetH. Reduced PduO levels or activity may enhance intracellular methylcobalamin levels and hence the availability of methylcobalamin to overexpressed MetH and hence methionine production. Increased expression of one or more of BtuC, BtuD and BtuF (e.g., increased production of BtuC, BtuD and BtuF) may increase methionine production.

TABLE 5a
B12 Uptake & Metabolism

| Gene | Protein |
|---|---|
| btuC | ABC-type vitamin B12 transporter permease |
| | (BtuC) |
| btuD | ABC-type vitamin B12 transporter ATPase |
| | (BtuD) EC 3.6.3.33 |
| btuF | ABC-type cobalamin/Fe³⁺ siderophore |
| | transporter (BtuF) |
| pduO | Adenosyltransferase (PduO) EC 4.2.1.28 |

Vitamin B12 Uptake

Vitamin B12 (cyanocobalamin) serves as a precursor to methylcobalamin, which is an essential cofactor in MetH catalyzed methylation of homocysteine to yield methionine. The following enzymes function in the uptake of vitamin B12 and related compounds from the bacterial environment.
ABC-type vitamin B12 transporter, permease component (BtuC)
ABC-type vitamin B12 transporter, ATPase component (BtuD)
ABC-type cobalamin/Fe³⁺ siderophore transport system (BtuF)

BtuC, BtuD, and BtuF, function in intracellular import of B12 and related compounds. Vitamin B12 serves as a precursor to methylcobalamin, which is a cofactor in the MetH catalyzed methylation of homocysteine to yield methionine. Thus, increased BtuC, BtuD, and/or BtuF expression or activity can increase methionine or S-adenosyl L-methionine production.

Cobalamin Adenosyltransferase

PduO catalyzes an adenosyltransferase reaction required to generate adenosylcobalamin from vitamin B12 (cyanocobalamin). Adenosylcobalamin is required by some vitamin B12-dependent enzymes, but not MetH (which requires methylcobalamin). Reduced levels or activity of PduO may increase the levels of methylcobalamin, due to increased availability of its precursor vitamin B12. As methylcobalamin is essential for MetH catalyzed conversion of homocysteine to methionine, increased levels of methylcobalamin may enhance methionine or S-adenosyl L-methionine production.

Glycine Cleavage System

Methyltetrahydrofolate provides the methyl group for the conversion of homocysteine to methionine catalyzed by MetH or MetE. Regeneration of methyltetrahydrofolate involves serine hydroxymethyltransferase (GlyA), tetrahydrofolate and serine and yields methylenetetrahydrofolate and glycine. In C. glutamicum fermentations glycine accumulates at levels near equimolar to methionine. However, in E. coli and many other bacteria (and plants and animals) glycine can serve as a substrate for additional regeneration of methytetrahydrofolate via the multi-enzyme glycine-cleavage system. Thus, expressing/overexpressing one or more of the genes required for the glycine-cleavage system may facilitate use of the excess glycine to regenerate methyltetrahydrofolate and thus may enhance methionine production. The proteins in the glycine cleavage system include the proteins listed in Table 5b.

TABLE 5b
Glycine Cleavage System

| Gene | Enzyme | Step |
|---|---|---|
| GcvP | Glycine dehydrogenase | Pyridoxal-phosphate |
| | (decarboxylating) | dependent decarboxylation |
| | | of glycine and transfer of |
| | | aminomethyl to gcvH |
| GcvH | H-protein; contains covalently | Carrier of aminomethyl |
| | attached lipoyl cofactor that functions | intermediate |
| | as carrier of an aminomethyl moiety | |
| GcvT | Aminomethyl transferase | Transfer of aminomethyl |
| | | C1 to tetrahydrofolate and |
| | | release of NH3 |
| LpdA | Dihydrolipoamide dehydrogenase | Reoxidizes GcvH lipoyl |
| | | prosthetic group |
| LplA | Lipoate-protein ligase A | Lipoylation of GcvH (and |
| | | other proteins) |
| LipA | Lipoic acid synthase | Synthesis of lipoic acid |
| LipB | lipoyl-[acyl-carrier-protein]-protein- | Transfer of lipoic acid to |
| | N-lipoyltransferase | GcvH (and other proteins) |
| The glycine-cleavage (GCV) system is a multi-enzyme complex that catalyzes the reversible oxidation of glycine, yielding carbon dioxide, methylenetetrahydrofolate, ammonia and a reduced pyridine nucleotide. The system is composed of P-(gcvP), H-(gcvH), T-(gcvT) and L-(lpdA) proteins. The H-protein contains a covalently attached lipoyl cofactor that functions as carrier of the glycine-derived aminomethyl moiety. The | | |
| # generation and attachment of the lipoyl cofactor to GcvH is facilitated by either LplA or LipA and LipB as listed in Table 5B. Although C. glutamicum lacks gcvP, gcvH and gcvT homologs it possesses homologs of proteins which may function in reoxidizing, generation and attachment of the lipoyl cofactor to GcvH. | | |

Additional Polypeptides

Additional biosynthetic, regulatory and transport polypeptides which can be used in combination with those described above are detailed below. Genetically engineered strains containing combinations of nucleic acid molecules encoding the various polypeptides can exhibit improved production of one or more amino acids or intermediates.

As noted above, pathways for precursors and co-factors used in methionine biosynthesis are important for determining the level of methionine production, and thus increasing expression and/or activity of any of the polypeptides that influence the supply of methionine pathway precursors and cofactors can lead to increased production of methionine and related amino acids and metabolites.

Exemplary polypeptides which can be used to enhance production of methionine, other aspartate family amino acids and metabolites and their corresponding SEQ ID NOs are provided in Table 6. The sequences that can be expressed in a host strain are not limited to those listed in Table 6. Thus, proteins having the same activity (i.e., homologs) from other species can be used as can variants of the listed polypeptides and their homologs.

TABLE 6
Examples of polypeptides involved in the production of methionone
and other aspartate family amino acids and metabolites

| | Bacterial | EC |
|---|---|---|
| Polypeptide | Gene | Number |
| Sulfate ABC transporter ATP-binding protein (permease A protein) | cysA | 3.6.3.25 |
| Sulfate transport system permease W protein | cysW | |
| Sulfate, thiosulfate transport system permease T protein | cysT | |
| Sulfate adenylyltransferase subunit 1 | cysN | 2.7.7.4 |
| Sulfate adenylyltransferase subunit 2 | cysD | 2.7.7.4 |
| Adenylylsulfate kinase | cysC | 2.7.1.25 |
| Phosphoadenosine phosphosulfate reductase | cysH | 1.8.1.48 |
| Sulfite reductase (alpha subunit or hemoprotein beta-component) | cysI | 1.8.1.2 |
| Sulfite reductase (NADPH), flavoprotein beta subunit in Ec (not found in | cysJ | 1.8.1.2 |
| Cg) | | |
| Adenylylsulphate reductase (assimilatory-type) | cysH | 1.8.99.2 |
| Phosphoglycerate dehydrogenase | serA | 1.1.1.95 |
| Phosphoserine transaminase | serC | 2.6.1.52 |
| Phosphoserine phosphatase | serB | 3.1.3.3 |
| Seine O-acetyltransferase | cysE | 2.3.1.30 |
| Cysteine synthase A | cysK | 2.5.1.47 |
| Cysteine synthase B | cysM | 2.5.1.47 |
| ABC-type vitamin B12 transporter, permease component | btuC | |
| ABC-type vitamin B12 transporter, ATPase component | btuD | 3.6.3.33 |
| ABC-type cobalamin/Fe3+-siderophore transport system | btuF | |
| Adenosyltransferase | pduO | |
| GTP cyclohydrolase I | folE | 4.2.1.28 |
| Phosphatase | phoA, psiA, | 3.5.4.16 |
| | psiF | |
| Dihydroneopterin aldolase | folB, ygiG | 3.1.3.1 |
| | | (3.6.1.—) |
| 7,8-dihydro-6-hydroxymethylpterin-pyrophosphokinase | folK | 4.1.2.25 |
| Dihydropteroate synthase | folP, dhpS | 2.7.6.3 |
| Dihydrofolate synthetase | folC, dedC | 2.5.1.15 |
| Dihydrofolate reductase | folA, tmrA, | 6.3.2.12 |
| | tmr | |
| Folylpolyglutamate synthetase (same as DHFS above) | folC, dedC | 1.5.1.3 |
| Putative methionine permease | yjeH | 6.3.2.17 |
| Transcriptional activator (of MetE/H) | metR | |
| 6-phosphogluconate dehydrogenase | gnd | |
| S-methylmethionine:homocysteine methyltransferase | mmuM | 1.1.1.44 |
| S-adenosylhomocysteme hydrolase | sahH | 2.1.1.10 |
| Site-specific DNA methylase | cglIM | 3.3.1.1 |
| Methionine export system protein 1 | brnF | 2.1.1.37 |
| Methionine export system protein 2 | brnE | |
| Aspartokinase | lysC | |
| Aspartate semialdehyde dehydrogenase | asd | 2.7.2.4 |
| Homoserine dehydrogenase | hom | 1.2.1.11 |
| O-homoserine acetyl transferase | metA | 1.1.1.3 |
| O-acetylhomoserine sulthydrylase | metY | 2.3.1.31 |
| Cobalamin-dependent methionine synthase | metH | 2.5.1.49 |
| Cobalamin-independent methionine synthase | metE | 2.1.1.13 |
| Homoserine kinase | thrB | 2.1.1.14 |
| Methionine adenosyltransferase | metK | 2.7.1.39 |
| O-succinyl(acetyl)lhomoserine (thio)-lyase | metB | 2.5.1.6 |
| Cystathionine beta-lyase | metC | 2.5.1.48 |
| 5,10-Methylenetetrahydrofolate reductase | metF | 4.4.1.8 |
| Dihydrodipicolinate synthase | dapA | 1.7.99.5 |
| Pyruvate carboxylase | pyc | 4.2.1.52 |
| Glutamate dehydrogenase | gdh | |
| Diaminopimelate dehydrogenase | ddh | |
| Methionine and cysteine biosynthesis repressor | mcbR | 1.4.1.16 |
| Lysine exporter protein | lysE | |
| Phosphoenolpyruvate carboxykinase | pck | |
| Phosphoenolpyruvate carboxylase | ppc | 4.1.1.49 |
| ABC transport system ATP-binding protein | metN | 4.1.1.31 |
| ABC transport system permease protein | metP/metP | |
| ABC transport system substrate-binding protein | metQ | |
| Glycine dehydrogenase (decarboxylating) | GcvP | 1.4.4.2 |
| H-protein; contains covalently attached lipoyl cofactor that functions as | GcvH | |
| carrier of an aminomethyl moeity | | |
| Aminomethyl transferase | GcvT | 2.1.2.10 |
| Dihydrolipoamide dehydrogenase | LpdA | 1.8.1.4 |
| Lipoate-protein ligase A | LplA | 6.3.2.— |
| Lipoic acid synthase | LipA | 2.8.1.— |
| lipoyl-[acyl-carrier-protein]-protein-N-lipoyltransferase | LipB | 2.3.1.— |
| fructose 1,6 bisphosphatase | fbp | 3.1.3.11 |
| glucose 6 phosphate dehydrogenase | g6pd | 1.1.1.49 |
| glucose-6-phosphate isomerase | pgi | 5.3.1.9 |
| NCgl2640 polypeptide | NCgl2640 | |

Methionine Biosynthesis Pathway

The enzymes in the methionine biosynhesis pathway and the steps they catalyze are described below (see also FIG. 1). Increasing the activity or expression of these enzymes can lead to increased methionine production. As described in detail below, some of the enzymes in the pathway can be mutated to reduce feedback inhitibion and thereby increase their activity.

Homoserine Dehydrogenase

Homoserine dehydrogenase (Hom) catalyzes the conversion of aspartate semialdehyde to homoserine. Hom is feedback-inhibited by threonine and repressed by methionine in coryneform bacteria. It is thought that this enzyme has greater affinity for aspartate semialdehyde than does the competing dihydrodipicolinate synthase (DapA) reaction in the lysine branch, but slight carbon “spillage” down the threonine pathway may still block Hom activity. Feedback-resistant variants of Hom, overexpression of hom, and/or deregulated transcription of hom, or a combination of any of these approaches, can enhance methionine, threonine, isoleucine, or S-adenosyl-L-methionine production. Decreased Hom activity can enhance lysine production. Bifunctional enzymes with homoserine dehydrogenase activity, such as enzymes encoded by E. coli metL (aspartokinase II-homoserine dehydrogenase II) and thrA (aspartokinase I-homoserine dehydrogenase I), can also be used to enhance amino acid production.

Homoserine O-Acetyltransferase

Homoserine O-acetyltransferase (MetA) acts at the first committed step in methionine biosynthesis (Park, S. et al., Mol. Cells 8:286-294, 1998). The MetA enzyme catalyzes the conversion of homoserine to O-acetyl-homoserine. MetA is strongly regulated by end products of the methionine biosynthetic pathway. In E. coli, allosteric regulation occurs by both S-AM and methionine, apparently at two separate allosteric sites. Moreover, MetJ and S-AM cause transcriptional repression of metA. In coryneform bacteria, MetA may be allosterically inhibited by methionine and S-AM, similarly to E. coli. MetA synthesis can be repressed by methionine alone. In addition, trifluoromethionine-resistance has been associated with metA in early studies. Reduction of negative regulation by S-AM and methionine can enhance methionine or S-adenosyl-L-methionine production. Increased MetA activity can enhance production of aspartate-derived amino acids such as methionine and S-AM, whereas decreased MetA activity can promote the formation of amino acids such as threonine and isoleucine.

O-Acetylhomoserine Sulfhydrylase

O-Acetylhomoserine sulfhydrylase (MetY) catalyzes the conversion of O-acetyl homoserine to homocysteine. MetY may be repressed by methionine in coryneform bacteria, with a 99% reduction in enzyme activity when grown in the presence of 0.5 mM methionine. In addition, enzyme activity is inhibited by methionine, homoserine, and O-acetylserine. It is possible that S-AM also modulates MetY activity. Deregulated MetY can enhance methionine or S-AM production.

Homoserine Kinase

Homoserine kinase is encoded by thrB gene, which is part of the hom-thrB operon. ThrB phosphorylates homoserine. Threonine inhibition of homoserine kinase has been observed in several species. Some studies suggest that phosphorylation of homoserine by homoserine kinase may limit threonine biosynthesis under some conditions. Increased ThrB activity can enhance production of aspartate-derived amino acids such as isoleucine and threonine, whereas decreased ThrB activity can promote the formation of amino acids including, but not limited to, lysine and methionine.

Methionine Adenosyltransferase

Methionine adenosyltransferase converts methionine to S-adenosyl-L-methionine (S-AM). Down-regulating methionine adenosyltransferase (MetK) can enhance production of methionine by inhibiting conversion to S-AM. Enhancing expression of metK or activity of MetK can maximize production of S-AM.

O-Succinylhomoserine (thio)-lyase/O-acetylhomoserine (thio)-lyase

O-Succinylhomoserine (thio)-lyase (MetB; also known as cystathionine gamma-synthase) catalyzes the conversion of O-succinyl homoserine or O-acetyl homoserine to cystathionine. Increasing expression or activity of MetB can lead to increased methionine or S-AM.

Cystathionine beta-lyase

Cystathionine beta-lyase (MetC) can convert cystathionine to homocysteine. Increasing production of homocysteine can lead to increased production of methionine. Thus, increased MetC expression or activity can increase methionine or S-adenosyl-L-methionine production.

5-Methyltetrahydrofolate Homocysteine Methyltransferase

5-Methyltetrahydrofolate homocysteine methyltransferase (MetH) catalyzes the conversion of homocysteine to methionine. This reaction is dependent on cobalamin (vitamin B12). Increasing MetH expression or activity can lead to increased production of methionine or S-adenosyl-L-methionine.

5-Methyltetrahydropteroyltriglutamate-homocysteine methyltransferase

5-Methyltetrahydropteroyltriglutamate-homocysteine methyltransferase (MetE) also catalyzes the conversion of homocysteine to methionine. Increasing MetE expression or activity can lead to increased production of methionine or S-adenosyl-L-methionine.

5,10-Methylenetetrahydrofolate Reductase

5,10-Methylenetetrahydrofolate reductase (MetF) catalyzes the reduction of methylenetetrahydrofolate to methyltetrahydrofolate, a cofactor for homocysteine methylation to methionine. Increasing expression or activity of MetF can lead to increased methionine or S-adenosyl-L-methionine production.

S-methylmethionine:Homocysteine Methyltransferase

S-methylmethionine:homocysteine methyltransferase (Mmum) catalyzes the transmethylation of homocysteine by S-methylmethionine to yield to yield methionine. Increasing the activity and/or expression of Mmum can therefore increase methionine or S-adenosyl L-methionine biosynthesis.

S-adenosylhomocysteine Hydrolase

S-adenosylhomocysteine hydrolase (SahH) catalyzes the reversible cleavage of S-adenosylhomocysteine, the side product of SAM-mediated methylation reactions, into adenosine and homocysteine, a precursor to methionine. Increasing the activity and/or expression of SahH can therefore increase methionine production. Overexpression of SahH can lead to the accumulation of other aspartate-derived amino acids such as lysine.

Site-Specific DNA Methylase

The site-specific DNA methylase (CglM) transfers the methyl group from S-adenosyl-L-methionine to DNA, resulting in the formation of S-adenosyl-L-homocysteine. Depending on the genetic context, either increasing or decreasing the expression of the site-specific DNA methylase can increase methionine or S-adenosyl-L-methionine production.

Proteins Involved in Supplying Metabolic Precursors and Reducing Equivalents Required for the Biosynthesis of Aspartate-Derived Amino Acids

Aspartokinases and Aspartate Semialdehyde Dehydrogenase

Aspartokinases (also referred to as aspartate kinases) are enzymes that catalyze the first committed step in the biosynthesis of aspartic acid family amino acids. The level and activity of aspartokinases are typically regulated by one or more end products of the pathway (lysine or lysine plus threonine depending upon the bacterial species), both through feedback inhibition (also referred to as allosteric regulation) and transcriptional control (also called repression). Bacterial homologs of coryneform and E. coli aspartokinases can be used to enhance amino acid production. Coryneform and E. coli aspartokinases can be expressed in heterologous organisms to enhance amino acid production. In Coryneform bacteria, aspartokinase is encoded by the lysC locus. The lysC locus contains two overlapping genes, lysC alpha and lysC beta. LysC alpha and lysC beta code for the 47- and 18-kD subunits of aspartokinase, respectively. A third open-reading frame is adjacent to the lysC locus, and encodes aspartate semialdehyde dehydrogenase (asd). The asd start codon begins 24 base-pairs downstream from the end of the lysC open-reading frame, is expressed as part of the lysC operon.

The primary sequence of aspartokinase proteins and the structure of the lysC loci are conserved across several members of the order Actinomycetales. Examples of organisms that encode both an aspartokinase and an aspartate semialdehyde dehydrogenase that are highly related to the proteins from coryneform bacteria include Mycobacterium smegmatis, Amycolatopsis mediterranei, Streptomyces coelicolor A3(2), and Thermobifida fusca. In some instances these organisms contain the lysC and asd genes arranged as in coryneform bacteria. Table 7 displays the percent identity of proteins from these Actinomycetes to the C. glutamicum aspartokinase and aspartate semialdehyde dehydrogenase proteins.

TABLE 7
Percent Identity of Heterologous Aspartokinase and Aspartate
Semialdehyde Dehydrogenase Proteins to C. glutamicum Proteins

| | Aspartokinase | Aspartate Semialdehyde |
|---|---|---|
| | (% Identity to | Dehydrogenase |
| | C. glutamicum | (% Identity to |
| Organism | LysC) | C. glutamicum Asd) |
| Mycobacterium smegmatis | 73 | 68 |
| Amycolatopsis mediterranei | 73 | 62 |
| Streptomyces coelicolor | 64 | 50 |
| Thermobifida fusca | 64 | 48 |

Isolates of source strains such as Mycobacterium smegmatis, Amycolatopsis mediterranei, Streptomyces coelicolor, and Thermobifida fusca are available. The lysC operons can be amplified from genomic DNA prepared from each source strain, and the resulting PCR product can be ligated into an E. coli/C. glutamicum shuttle vector. The homolog of the aspartokinase enzyme from the source strain can then be introduced into a host strain and expressed.

In coryneform bacteria there is concerted feedback inhibition of aspartokinase by lysine and threonine. This is in contrast to E. coli, where there are three distinct aspartokinases that are independently allosterically regulated by lysine, threonine, or methionine. Homologs of the E. coli aspartokinase III (and other isoenzymes) can be used as an alternative source of deregulated aspartokinase proteins. Expression of these enzymes in coryneform bacteria may decrease the complexity of pathway regulation. For example, the aspartokinase III genes are feedback-inhibited only by lysine instead of lysine and threonine. Therefore, the advantages of expressing feedback-resistant alleles of aspartokinase III alleles include: (1) the increased likelihood of complete deregulation; and (2) the possible removal of the need for constructing either “leaky” mutations in hom or threonine auxotrophs that need to be supplemented. These features can result in decreased feedback inhibition by lysine.

Genes encoding aspartokinase III isoenzymes can be isolated from bacteria that are more distantly related to Corynebacteria than the Actinomycetes described above. For example, the E. chysanthemi and S. oneidensis gene products are 77% and 60% identical to the E. coli lysC protein, respectively (and 26% and 35% identical to C. glutamicum LysC). The genes coding for aspartokinase III, or functional variants therof, from the non-Escherichia bacteria, Erwinia chrysanthemi and Shewanella oneidensis can be amplified and ligated into the appropriate shuttle vector for expression in C. glutamicum.

Dihydrodipicolinate Synthases

Dihydrodipicolinate synthase, encoded by dapA, is the branch point enzyme that commits carbon to lysine biosynthesis rather than threonine/methionine production. DapA converts aspartate-β-semialdehyde to 2,3-dihydrodipicolinate. DapA overexpression has been shown to result in increased lysine production in both E. coli and coryneform bacteria. In E. coli, DapA is allosterically regulated by lysine, whereas existing evidence suggests that C. glutamicum regulation occurs at the level of gene expression. Dihydrodipicolinate synthase proteins are not as well conserved amongst Actinomycetes as compared to LysC proteins.

Both wild-type and deregulated DapA proteins that are homologous to the C. glutamicum protein or the E. coli DapA protein can be expressed to enhance lysine production. Candidate organisms that can be sources of dapA genes are shown in Table 8. The known sequence from M. tuberculosis or M. leprae can be used to identify homologous genes from M. smegmatis.

TABLE 8
Percent Identity of Dihydrodipicolinate Synthase Proteins.

| | % Identity to | % Identity to |
|---|---|---|
| Organism | C. glutamicum DapA | E. coli DapA |
| Corynebacterium glutamicum | 100 | 34 |
| Mycobacterium tuberculosis | 59 | 33 |
| H37Rv* | | |
| Streptomyces coelicolor | 53 | 33 |
| Thermobifida fusca | 48 | 33 |
| Erwinia chrysanthemi | 34 | 81 |

*Can be used for cloning of the M. smegmatis dapA gene.

Amino acid substitutions that relieve feedback inhibition of E. coli DapA by lysine have been described. Examples of such substitutions are listed in Table 5. Some of the residues that can be altered to relieve feedback inhibition are conserved in all of the candidate DapA proteins (e.g. Leu 88, His 118). This sequence conservation suggests that similar substitutions in the proteins from Actinomycetes may further enhance protein function in the presence of normally inhibitory levels of lysine. Site-directed mutagenesis can be employed to engineer deregulated DapA variants.

DapA isolates can be tested for increased lysine production using methods described above. For instance, one could distribute a culture of a lysine-requiring bacterium on a growth medium lacking lysine. A population of dapA mutants obtained by site-directed mutagenesis could then be introduced (through transformation or conjugation) into a wild-type coryneform strain, and subsequently spread onto the agar plate containing the distributed lysine auxotroph. A feedback-resistant dapa mutant would overproduce lysine which would be excreted into the growth medium and satisfy the growth requirement of the auxotroph previously distributed on the agar plate. Therefore a halo of growth of the lysine auxotroph around a dapa mutation-containing colony would indicate the presence of the desired feedback-resistant mutation.

In order to increase the production of aspartate-derived amino acids that use homoserine as a biosynthetic intermediate, it may be useful to decrease DapA activity. Diaminopimelate is essential for viability in some bacteria, including corynebacteria. Therefore, strain construction may require the introduction of a “leaky” dapa allele, meaning an allele that allows for growth without allowing for any excess carbon flow into the lysine biosynthetic pathway.

TABLE 9
Amino Acid Substitutions in Dihydrodipicolinate Synthase That Release
Feedback Inhibition.

| | Amino Acid Substitution (using E. coli |
|---|---|
| Organism | DapA amino acid # as reference |
| Glycine max | Asn 80 [symbol] Ile |
| Nicotiana sylvestris | |
| Escherichia coli | Ala 81 [symbol] Val |
| Zea mays | Glu 84 [symbol] Lys |
| Methylobacillus glycogens | Leu 88 [symbol] Phe |
| Escherichia coli | His 118 [symbol] Tyr |

Pyruvate and Phosphoenolpyruvate Carboxylases

Pyruvate carboxylase (Pyc) and phosphoenolpyruvate carboxylase (Ppc) catalyze the synthesis of oxaloacetic acid (OAA), the citric acid cycle intermediate that feeds directly into lysine biosynthesis. These anaplerotic reactions have been associated with improved yields of several amino acids, including lysine, and are obviously important to maximize OAA formation. In addition, a variant of the C. glutamicum Pyc protein containing a P458S substitution, has been shown to have increased activity, as demonstrated by increased lysine production. Proline 458 is a highly conserved amino acid position across a broad range of pyruvate carboxylases, including proteins from the Actinomycetes S. coelicolor (amino acid residue 449) and M. smegmatis (amino acid residue 448). Similar amino acid substitutions in these proteins may enhance anaplerotic activity. A third gene, PEP carboxykinase (pck), expresses an enzyme that catalyzes the formation of phosphoenolpyruvate from OAA (for gluconeogenesis), and thus functionally competes with pyc and ppc. Enhancing expression of pyc and ppc can maximize OAA formation. Reducing or eliminating pck activity can also improve OAA formation.

6-Phosphogluconate Dehydrogenase (Gnd)

6-phosphogluconate dehydrogenase catalyzes the oxidation and decarboxylation of 6-phosphogluconate to D-ribulose-5-phosphate. This reaction also regenerates NADPH, which is required for a variety of reductive biosyntheses, including the formation of aspartate-derived amino acids. Enhancing expression of gnd or activity of Gnd can improve the production of aspartate-derived amino acids, including methionine.

Fructose 1,6 Bisphophatase (fbp)

Fructose 1,6 bisphophatase is a hydrolase which catalyses the reaction of D-fructose 1,6-bisphosphate+H₂O→D-fructose 6-phosphate+phosphate. Fructose 1,6 bisphophatase activity can enhance flux through the pentose phosphate pathway which is a major metabolic pathway of NADPH production. As stated above, NADPH is required for a variety of reductive biosyntheses, including the formation of aspartate-derived amino acids. fbp overexpression has been reported to result in increased lysine production in C. glutamicum (Becker et al. Appl Environ Micrbiol. 2005 71:8587-96). Thus, enhancing expression of fbp or activity of fructose 1,6 bisphophatase can improve the production of aspartate-derived amino acids, including methionine.

Glucose 6 Phosphate Dehydrogenase (g6pd)

Glucose 6 phosphate dehydrogenase functions as part of the pentose phosphate pathway and catalyses the reaction of D-glucose 6-phosphate+NADP⁺→D-glucono-1,5-lactone 6-phosphate+NADPH+H⁺. Thus, enhancing the expression of g6pd or the activity of glucose 6 phosphate dehydrogenase increases NADPH levels and can improve the production of aspartate-derived amino acids, including methionine.

Glucose-6-phosphate Isomerase (pgi) glucose-6-phosphate isomerase functions during glycolysis and converts D-glucose 6-phosphate to D-fructose 6-phosphate. Thus, reduction or elimination of pgi activity inhibits glucose catabolism via the Embden-Meyerhof Pathway (glycolysis). pgi deletion mutants in C. glutamicum exhibit increased flux through the alternative glucose catabolism pathway (the pentose phosphate pathway), increased NADPH production and increased lysine production (Marx et al. 2003 J Biotechnol 104:185-97). Thus, reducing or eliminating expression of pgi or activity of glucose-6-phosphate isomerase increases NADPH levels and can improve the production of aspartate-derived amino acids, including methionine.

Glutamate Dehydrogenase

The enzyme glutamate dehydrogenase, encoded by the gdh gene, catalyses the reductive amination of α-ketoglutarate to yield glutamic acid. In coryneform bacteria, this reaction requires NADPH. In some instances, increasing expression or activity of glutamate dehydrogenase can lead to increased lysine, threonine, isoleucine, valine, proline, or tryptophan. In other cases, reduced activity can result in increased production of aspartate-derived amino acids, either due to the increased availability of NADPH reducing equivalents or the decreased carbon drain of tricarboxylic pathway intermediates.

Diaminopimelate Dehydrogenase

Diaminopimelate dehydrogenase, encoded by the ddh gene in coryneform bacteria, catalyzes the the NADPH-dependent reduction of ammonia and L-2-amino-6-oxopimelate to form meso-2,6-diaminopimelate, the direct precursor of L-lysine in the alternative pathway of lysine biosynthesis. Overexpression of diaminopimelate dehydrogenase can increase lysine production. Decreased activity could result in enhanced production of homoserine-derived amino acids such as methionine.

Regulatory Proteins

McbR Gene Product

The mcbR gene product of C. glutamicum was identified as a putative transcriptional repressor of the TetR-family and may be involved in the regulation of the metabolic network directing the synthesis of methionine in C. glutamicum (Rey et al., J Biotechnol. 103(1):51-65, 2003). The mcbR gene product represses expression of metY, metK, cysK, cysI, hom, pyk, ssuD, and possibly other genes. It is possible that McbR represses expression in combination with small molecules such as S-adenosylhomocysteine, S-AM or methionine. To date specific alleles of McbR that prevent binding of either S-adenosylhomocysteine, S-AM or methionine have not been identified. Reducing expression of McbR, and/or preventing regulation of McbR by S-adenosylhomocysteine, S-AM or methionine can enhance amino acid production.

McbR is involved in the regulation of sulfur containing amino acids (e.g., cysteine, methionine). Reduced McbR expression or activity can also enhance production of any of the aspartate family of amino acids that are derived from homoserine (e.g., homoserine, O-acetyl-L-homoserine, O-succinyl-L-homoserine, cystathionine, L-homocysteine, L-methionine, S-adenosyl-L-methionine (S-AM), O-phospho-L-homoserine, threonine, 2-oxobutanoate, (S)-2-aceto-2-hydroxybutanoate, (S)-2-hydroxy-3-methyl-3-oxopentanoate, (R)-2,3-Dihydroxy-3-methylpentanoate, (R)-2-oxo-3-methylpentanoate, and L-isoleucine).

MetR Gene Product

The MetR gene product is a transcriptional activator of the MetE and MetH genes in E. coli. Increasing expression of the MetR gene product can lead to increased expression of MetE and MetH gene products and thereby increase methionine biosynthesis.

Ncgl2640 Gene Product

The Ncgl2640 gene product shows some homology to the glutamate-cysteine ligase family 2. The archetype enzyme of this family catalyzes the first step in de novo glutathione biosynthesis. Mampel et al. (Appl Microbiol Biotechnol. 200568:228-36.) observed transposon insertion inactivation of NCgl2640 in C. glutamicum correlates with increased methionine production and relief of L-methionine repression of cysteine synthase, o-acetylhomoserine sulfhydrolase (mety) and sulfite reductase. Thus decreasing or eliminating expression of Ncgl2640 or activity of the Ncgl264 gene product can improve the production of aspartate-derived amino acids, including methionine.

Efflux Proteins

A substantial number of bacterial genes encode membrane transport proteins. A subset of these membrane transport protein mediate efflux of amino acids from the cell. For example, Corynebacterium glutamicum express a threonine efflux protein. Loss of activity of this protein leads to a high intracellular accumulation of threonine (Simic et al., J Bacteriol. 183(18):5317-5324, 2001). Modulating expression or activity of efflux proteins can lead to increased production of various amino acids and related metabolites. Useful efflux proteins include proteins of the drug/metabolite transporter family.

Detergent Sensitivity Rescuer

Detergent sensitivity rescuer (dtsR1), encoding a protein related to the alpha subunit of acetyl CoA carboxylase, is a surfactant resistance gene. Increasing expression or activity of DtsR1 can lead to increased production of lysine. Increased expression may also lead to increased production of other aspartate-derived amino acids.

Lysine Exporter Protein

Lysine exporter protein (LysE) is a specific lysine translocator that mediates efflux of lysine from the cell. In C. glutamicum with a deletion in the lysE gene, L-lysine can reach an intracellular concentration of more than 1M. (Erdmann, A., et al. J Gen Microbiol. 139,:3115-3122, 1993). Overexpression or increased activity of this exporter protein can enhance lysine production. Decreased LysE activity can enhance the production of non-lysine, aspartate-derived amino acids.

YjeH

yjeH encodes an E. coli protein involved in the transport of methionine. Increased expression of YjeH can enhance methionine production. Increased expression of YjeH can also lead to enhanced production of methionine pathway intermediates.

BrnFE

BrnFE is a two-component export system comprised of the BmF (AzlC) and BmE (AzlD) polypeptides. Overexpression of BrnFE (i.e., overexpression of BmF and BmE) can lead to the enhanced export of branched-chain amino acids, including isoleucine. Increased expression of BrnFE can also enhance methionine production.

MetD

MetD is a high affinity methionine uptake systrem of the ABC-type transporter family and is comprised of MetNPQ. MetN is the ATP-binding protein, MetP is the permease protein (metI is a likely functional equivalent), and MetQ is the substrate-binding protein. Reduced expression or inactivation of the MetD uptake system can reduce methionine uptake, which can result in increased methionine production.

Bacterial Host Strains

Suitable host species for the production of amino acids include bacteria of the family Enterobacteriaceae such as an Escherichia coli bacteria and strains of the genus Corynebacterium. The list below contains examples of species and strains that can be used as host strains for the expression of heterologous and/or homologous genes and for the production of amino acids and related intermediates and metabolites.

Escherichia coli W3110 F⁻IN(rrnD-rrnE)1 λ⁻(E. coli Genetic Stock Center)

Corynebacterium glutamicum ATCC (American Type Culture Collection) 13032

Corynebacterium glutamicum ATCC 21526

Corynebacterium glutamicum ATCC 21543

Corynebacterium glutamicum ATCC 21608

Corynebacterium acetoglutamicum ATCC 15806

Corynebacterium acetoglutamicum ATCC 21491

Corynebacterium acetoglutamicum NRRL B-11473

Corynebacterium acetoglutamicum NRRL B-11475

Corynebacterium acetoacidophilum ATCC 13870

Corynebacterium melassecola ATCC 17965

Corynebacterium thermoaminogenes FERM BP-1539

Brevibacterium lactis

Brevibacterium lactofermentum ATCC 13869

Brevibacterium lactofermentum NRRL B-11470

Brevibacterium lactofermentum NRRL B-11471

Brevibacterium lactofermentum ATCC 21799

Brevibacterium lactofermentum ATCC 31269

Brevibacterium flavum ATCC 14067

Brevibacterium flavum ATCC 21269

Brevibacterium flavum NRRL B-11472

Brevibacterium flavum NRRL B-11474

Brevibacterium flavum ATCC 21475

Brevibacterium divaricatum ATCC 14020

Bacterial Strains for use as a Source of Genes

Suitable species and strains from which nucleic acid sequences can be obtained include, but are not limited to those listed below

Amycolatopsis mediterranei

Bacillus halodurans

Bacillus sphaericus

Clostridium acetobutylicum

Corynebacterium diptheriae

Corynebacterium glutamicum

Escherichia coli

Erwinia chrysanthemi (e.g., ATCC 11663)

Erwinia Carotovora

Lactobacillus plantarum (e.g. ATCC 8014)

Mycobacterium avium

Mycobacterium bovis

Mycobacterium leprae

Mycobacterium smegmatis (e.g. ATCC 700084)

Mycobacterium tuberculosis (e.g. Mycobacterium tuberculosis H37Rv)

Nocardia farcinica

Shewanella oneidensis

Streptomyces coelicolor (e.g. Streptomyces coelicolor A3(2))

Thermobifida fusca (e.g. ATCC 27730)

Isolation of Bacterial Genes

Bacterial genes for expression in host strains can be isolated by methods known in the art. See, for example, Sambrook, J., and Russell, D. W. (Molecular Cloning: A Laboratory Manual, 3nd Ed., Cold Spring Harbor Laboratory Press, Cold Spring Harbor, N.Y., 2001) for methods of construction of recombinant nucleic acids. Genomic DNA from source strains can be prepared using known methods (see, e.g., Saito, H. and, Miura, K. Biochim Biophys Acta. 72:619-629, 1963) and genes can be amplified from genomic DNA using PCR (U.S. Pat. Nos. 4,683,195 and 4,683,202, Saiki, et al. Science 230:350-1354, 1985).

DNA primers to be used for the amplification reaction are those complementary to both 3′-terminals of a double stranded DNA containing an entire region or a partial region of a gene of interest. When only a partial region of a gene is amplified, it is necessary to use such DNA fragments as primers to perform screening of a DNA fragment containing the entire region from a chromosomal DNA library. When the entire region gene is amplified, a PCR reaction solution including DNA fragments containing the amplified gene is subjected to agarose gel electrophoresis, and then a DNA fragment is extracted and cloned into a vector appropriate for expression in bacterial systems.

DNA primers for PCR may be adequately prepared on the basis of, for example, a sequence known in the source strain (Richaud, F. et al., J. Bacteriol. 297,1986). For example, primers that can amplify a region comprising the nucleotide bases coding for the heterologous gene of interest can be used. Synthesis of the primers can be performed by an ordinary method such as a phosphoamidite method (see Tetrahed Lett. 22:1859,1981) by using a commercially available DNA synthesizer (for example, DNA Synthesizer Model 380B produced by Applied Biosystems Inc.). Further, the PCR can be performed by using a commercially available PCR apparatus and Taq DNA polymerase, or other polymerases that display higher fidelity, in accordance with a method designated by the supplier.

Construction of Variant Alleles

Many enzymes that regulate amino acid production are subject to allosteric feedback inhibition by biosynthetic pathway intermediates or end products. Useful variants of these enzymes can be generated by substitution of residues responsible for feedback inhibition.

Standard site-directed mutagenesis techniques can be used to construct variants that are less sensitive to allosteric regulation. After cloning a PCR-amplified gene or genes into appropriate shuttle vectors, oligonucleotide-mediated site-directed mutagenesis is use to provide modified alleles that encode specific amino acid substitutions. Vectors containing either wild-type genes or modified alleles can be transformed into C. glutamicum, or another suitable host strain, alongside control vectors. The resulting transformants can be screened, for example, for amino acid productivity, increased resistance of an enzyme to feedback inhibition, or other criteria known to those skilled in the art to identify the variant alleles of most interest. Assays to measure amino acid productivity and/or enzyme activity can be used to confirm the screening results and select useful variant alleles. Techniques such as high pressure liquid chromatography (HPLC) and HPLC-mass spectrometry (MS) assays to quantify levels of methionine and related metabolites are known to those skilled in the art.

Methods for generating random amino acid substitutions within a coding sequence, through methods such as mutagenic PCR, can be used (e.g., to generate variants for screening for reduced feedback inhibition, or for introducing further variation into enhanced variant sequences). For example, PCR can be performed using the GeneMorph® PCR mutagenesis kit (Stratagene, La Jolla, Calif.) according to manufacturer's instructions to achieve medium and high range mutation frequencies. Other methods are also known in the art.

Evaluation of enzymes can be carried out in the presence of additional enzymes that are endogenous to the host strain. In certain instances, it will be helpful to have reagents to specifically assess the functionality of a biosynthetic protein that is not endogenous to the organism (e.g., an episomally expressed protein). Phenotypic assays for feedback inhibition or enzyme assays can be used to confirm function of wild-type and variants of biosynthetic enzymes. The function of cloned genes can be confirmed by complementation of genetically characterized mutants of the host organism (e.g., the host E. coli or C. glutamicum bacterium). Many of the E. coli strains are publicly available from the E. coli Genetic Stock Center, which has a list of available strains on its site on the world wide web. C. glutamicum mutants have also been described.

Expression of Genes

Bacterial genes can be expressed in host bacterial strains using methods known in the art. In some cases, overexpression of a bacterial gene (e.g., a heterologous and/or variant gene) will enhance amino acid production by the host strain. Overexpression of a gene can be achieved in a variety of ways. For example, multiple copies of the gene can be expressed, or the promoter, regulatory elements, and/or ribosome binding site upstream of a gene (e.g., a variant allele of a gene, or an endogenous gene) can be modified for optimal expression in the host strain. In addition, the presence of even one additional copy of the gene can achieve increased expression, even where the host strain already harbors one or more copies of the corresponding gene native to the host species. The gene can be operably linked to a strong constitutive promoter or an inducible promoter (e.g., trc, lac) and induced under conditions that facilitate maximal amino acid production. Methods to enhance stability of the mRNA are known to those skilled in the art and can be used to ensure consistently high levels of expressed proteins. See, for example, Keasling, J., Trends in Biotechnology 17:452-460, 1999. Optimization of media and culture conditions may also enhance expression of the gene.

Methods for facilitating expression of genes in bacteria have been described. See, for example, Guerrero, C, et al., Gene 138(1-2): 35-41, 1994; Eikmanns, B. J., et al. Gene 102(1): 93-8, 1991; Schwarzer, A., and Puhler, A. Biotechnol. 9(1): 84-7, 1991; Labarre, J., et al., J Bacteriol. 175(4): 1001-7, 1993; Malumbres, M., et al. Gene 134(1):15-24, 1993; Jensen, P. R., and Hammer, K. Biotechnol Bioeng. 158(2-3): 191-5, 1998; Makrides, S. C. Microbiol Rev. 60(3): 512-38, 1996; Tsuchiya et al. Bio/Technology 6:428-431,1988; U.S. Pat. No. 5,965,931; U.S. Pat. No. 4,601,893; and U.S. Pat. No. 5,175,108.

A gene of interest (e.g., a heterologous or variant gene) should be operably linked to an appropriate promoter, such as a native or host strain-derived promoter, a phage promoter, one of the well-characterized E. coli promoters (e.g. tac, trp, phoA, araBAD, or variants thereof etc.). Other suitable promoters are also available. In one embodiment, the heterologous gene is operably linked to a promoter that permits expression of the heterologous gene at levels at least 2-fold, 5-fold, or 10-fold higher than levels of the endogenous homolog in the host strain. Plasmid vectors that aid the process of gene amplification by integration into the chromosome can be used. See, for example, Reinscheid et al. (Appl. Environ Microbiol. 60: 126-132,1994). In this method, the complete gene is cloned in a plasmid vector that can replicate in a host (typically E. coli), but not in C. glutamicum. These vectors include, for example, pSUP301 (Simon et al., Bio/Technol. 1, 784-79,1983), pK18mob or pK19mob (Schfer et al., Gene 145:69-73, 1994), PGEM-T (Promega Corp., Madison, Wisc., USA), pCR2.1-TOPO (Shuman J Biol Chem. 269:32678-84, 1994; U.S. Pat. No. 5,487,993), pCR.RTM.Blunt (Invitrogen, Groningen, Holland; Bernard et al., J Mol Biol., 234:534-541,1993), pEM1 (Schrumpf et al. J Bacteriol. 173:4510-4516, 1991) or pBGS8 (Spratt et al., Gene 41:337-342, 1996). The plasmid vector that contains the gene to be amplified is then transferred into the desired strain of C. glutamicum by conjugation or transformation. The method of conjugation is described, for example, by Schfer et al. (Appl Environ Microbiol. 60:756-759,1994). Methods for transformation are described, for example, by Thierbach et al. (Appl Microbiol Biotechnol. 29:356-362,1988), Dunican and Shivnan (Bio/Technol. 7:1067-1070,1989) and Tauch et al. (FEMS Microbiol Lett. 123:343-347,1994). After homologous recombination by means of a genetic cross over event, the resulting strain contains the desired gene integrated in the host genome.

An appropriate expression plasmid can also contain at least one selectable marker. A selectable marker can be a nucleotide sequence that confers antibiotic resistance in a host cell. These selectable markers include ampicillin, cefazolin, augmentin, cefoxitin, ceftazidime, ceftiofur, cephalothin, enrofloxicin, kanamycin, spectinomycin, streptomycin, tetracycline, ticarcillin, tilmicosin, or chloramphenicol resistance genes. Additional selectable markers include genes that can complement nutritional auxotrophies present in a particular host strain (e.g. leucine, alanine, or hornoserine auxotrophies).

In one embodiment, a replicative vector is used for expression of the heterologous gene. An exemplary replicative vector can include the following: a) a selectable marker, e.g., an antibiotic marker, such as kanR (from pACYC184); b) an origin of replication in E. coli, such as the P15a ori (from pACYC184); c) an origin of replication in C. glutamicum such as that found in pBL1; d) a promoter segment, with or without an accompanying repressor gene; and e) a terminator segment. The promoter segment can be a lac, trc, trcRBS, tac, or λP_{L}/λP_{R} (from E. coli), or phoA, gpd, rplM, rpsJ (from C. glutamicum). The repressor gene can be lacI or cI857, for lac, trc, trcRBS, tac and λP_{L}/λP_{R}, respectively. The terminator segment can be from E. coli rrnB (from ptrc99a), the T7 terminator (from pET26), or a terminator segment from C. glutamicum.

In another embodiment, an integrative vector is used for expression of the heterologous gene. An exemplary integrative vector can include: a selectable marker, e.g., an antibiotic marker, such as kanR (from pACYC 184); b) an origin of replication in E. coli, such as the P15a ori (from pACYC 184); c) and d) two segments of the C. glutamicum genome that flank the segment to be replaced, such as the pck or horn genes; e) the sacB gene from B. subtilis; f) a promoter segment to control expression of the heterologous gene, with or without an accompanying repressor gene; and g) a terminator segment. The promoter segment can be lac, trc, trcRBS, tac, or λP_{L}/λP_{R} (from E. coli), or phoA, gpd, rplM, rpsJ (from C. glutamicum). The repressor genes can be lacI or cI, for lac, trc, trcRBS, tac and λP_{L}/λP_{R}, respectively. The terminator segment can be from E. coli rrnB (from ptrc99a), the T7 terminator (from pET26), or a terminator segment from C. glutamicum. The possible integrative or replicative plasmids, or reagents used to construct these plasmids, are not limited to those described herein. Other plasmids are familiar to those in the art.

For use of terminator segments from C. glutamicum, the terminator and flanking sequences can be supplied by a single gene segment. In this case, the above elements will be arranged in the following sequence on the plasmid: marker; origin of replication; a segment of the C. glutamicum genome that flanks the segment to be replaced; promoter; C. glutamicum terminator; sacB gene. The sacB gene can also be placed between the origin of replication and the C. glutamicum flanking segment. Integration and excision results in the insertion of only the promoter, terminator, and the gene of interest.

A multiple cloning site can be positioned in one of several possible locations between the plasmid elements described above in order to facilitate insertion of the particular genes of interest (e.g., lysC, etc.) into the plasmid. For both replicative and integrative vectors, the addition of an origin of conjugative transfer, such as RP4 mob, can facilitate gene transfer between E. coli and C. glutamicum.

In one embodiment, a bacterial gene is expressed in a host strain with an episomal plasmid. Suitable plasmids include those that replicate in the chosen host strain, such as a coryneform bacterium. Many known plasmid vectors, such as e.g. pZ1 (Menkel et al., Applied Environ Microbiol. 64:549-554, 1989), pEKE×1 (Eikmanns et al., Gene 102:93-98,1991) or pHS2-1 (Sonnen et al., Gene 107:69-74, 1991) are based on the cryptic plasmids pHM1519, pBL1 or pGA1. Other plasmid vectors that can be used include those based on pCG4 (U.S. Pat. No. 4,489,160), or pNG2 (Serwold-Davis et al., FEMS Microbiol Lett. 66:119-124,1990), or pAG1 (U.S. Pat. No. 5,158,891). Alternatively, the gene or genes may be integrated into chromosome of a host microorganism by a method using transduction, transposon (Berg, D. E. and Berg, C. M., Bio/Technol. 1:417,1983), Mu phage (Japanese Patent Application Laid-open No. 2-109985) or homologous or non-homologous recombination (Experiments in Molecular Genetics, Cold Spring Harbor Lab., 1972).

In addition, it may be advantageous for the production of amino acids to enhance one or more enzymes of the particular biosynthesis pathway, of glycolysis, of anaplerosis, or of amino acid export, using more than one gene or using a gene in combination with other biosynthetic pathway genes.

It also may be advantageous to simultaneously attenuate the expression of particular gene products to maximize production of a particular amino acid. For example, attenuation of metK expression or MetK activity can enhance methionine production by prevention conversion of methionine to S-AM.

Methods of introducing nucleic acids into host cells are known in the art. See, for example, Sambrook, J., and Russell, D. W. Molecular Cloning: A Laboratory Manual, 3^{nd} Ed., Cold Spring Harbor Laboratory Press, Cold Spring Harbor, N.Y., 2001. Suitable methods include transformation using calcium chloride (Mandel, M. and Higa, A. J. Mol Biol. 53:159, 1970) and electroporation (Rest, M. E. van der, et al. Appl Microbiol. Biotechnol. 52:541-545, 1999), or conjugation.

Cultivation of Bacteria

The bacteria containing gene(s) of interest (e.g., heterologous genes, variant genes encoding enzymes with reduced feedback inhibition) can be cultured continuously or by a batch fermentation process (batch culture). Other commercially used process variations known to those skilled in the art include fed batch (feed process) or repeated fed batch process (repetitive feed process). A summary of known culture methods is described in the textbook by Chmiel (Bioprozesstechnik 1. Einfuhrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used fulfills the requirements of the particular host strains. General descriptions of culture media suitable for various microorganisms can be found in the book “Manual of Methods for General Bacteriology” of the American Society for Bacteriology (Washington D.C., USA, 1981), although those skilled in the art will recognize that the composition of the culture medium is often modified beyond simple growth requirements in order to maximize product formation.

Sugars and carbohydrates, such as e.g., glucose, sucrose, lactose, fructose, maltose, starch and cellulose; oils and fats, such as e.g. soy oil, sunflower oil, groundnut oil and coconut fat; fatty acids, such as e.g. palmitic acid, stearic acid and linoleic acid; alcohols, such as e.g. glycerol and ethanol; and organic acids, such as e.g. acetic acid, can be used as the source of carbon, either individually or as a mixture.

Organic nitrogen-containing compounds, such as peptones, yeast extract, meat extract, malt extract, corn steep liquor, soy protein hydrolysate, soya bean flour and urea, or inorganic compounds, such as ammonium sulfate, ammonium chloride, ammonium phosphate, ammonium carbonate and ammonium nitrate, can be used as the source of nitrogen. The. sources of nitrogen can be used individually or as a mixture.

Phosphoric acid, potassium dihydrogen phosphate, dipotassium hydrogen phosphate, or the corresponding sodium-containing salts can be used as the source of phosphorus.

Organic and inorganic sulfur-containing compounds, such as, for example, sulfates, thiosulfates, sulfites, reduced sources such as H₂S, sulfides, derivatives of sulfides, methyl mercaptan, thioglycolytes, thiocyanates, and thiourea, can be used as sulfur sources for the preparation of sulfur-containing amino acids.

The culture medium can also include salts of metals, e.g., magnesium sulfate or iron sulfate, which are necessary for growth. Essential growth substances, such as amino acids and vitamins (e.g. cobalamin), can be employed in addition to the above-mentioned substances. Suitable precursors can moreover be added to the culture medium. The starting substances mentioned can be added to the culture as a single batch, or can be fed in during the culture at multiple points in time.

Basic compounds, such as sodium hydroxide, potassium hydroxide, calcium carbonate, ammonia or aqueous ammonia, or acid compounds, such as phosphoric acid or sulfuric acid, can be employed in a suitable manner to control the pH. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is typically between 20-45° C. and preferably 25-40° C. Culturing is continued until a maximum of the desired product has formed, usually within 10 hours to 160 hours.

The fermentation broths obtained in this way, can contain a dry weight of 2.5 to 25 wt. % of the amino acid of interest. It also can be advantageous if the fermentation is conducted in such that the growth and metabolism of the production microorganism is limited by the rate of carbohydrate addtion for some portion of the fermentation cycle, preferably at least for 30% of the duration of the fermentation. For example, the concentration of utilizable sugar in the fermentation medium is maintained at ≦3 g/l during this period.

The fermentation broth can then be further processed. All or some of the biomass can be removed from the fermentation broth by any solid-liquid separation method, such as centrifugation, filtration, decanting or a combination thereof, or it can be left completely in the broth. Water is then removed from the broth by known methods, such as with the aid of a multiple-effect evaporator, thin film evaporator, falling film evaporator, or by reverse osmosis. The concentrated fermentation broth can then be worked up by methods of freeze drying, spray drying, fluidized bed drying, or by other processes to give a preferably free-flowing, finely divided powder.

The free-flowing, finely divided powder can then in turn by converted by suitable compacting or granulating processes into a coarse-grained, readily free-flowing, storable and largely dust-free product. In the granulation or compacting it can be advantageous to use conventional organic or inorganic auxiliary substances or carriers, such as starch, gelatin, cellulose derivatives or similar substances, such as are conventionally used as binders, gelling agents or thickeners in foodstuffs or feedstuffs processing, or further substances, such as, for example, silicas, silicates or stearates.

Alternatively, however, the product can be absorbed on to an organic or inorganic carrier substance which is known and conventional in feedstuffs processing, for example, silicas, silicates, grits, brans, meals, starches, sugars or others, and/or mixed and stabilized with conventional thickeners or binders.

Finally, the product can be brought into a state in which it is stable to digestion by animal stomachs, in particular the stomach of ruminants, by coating processes using film-forming agents, such as, for example, metal carbonates, silicas, silicates, alginates, stearates, starches, gums and cellulose ethers, as described in DE-C-4100920.

If the biomass is separated off during the process, further inorganic solids, for example, those added during the fermentation, are generally removed.

In one aspect of the invention, the biomass can be separated off to the extent of up to 70%, preferably up to 80%, preferably up to 90%, preferably up to 95%, and particularly preferably up to 100%. In another aspect of the invention, up to 20% of the biomass, preferably up to 15%, preferably up to 10%, preferably up to 5%, particularly preferably no biomass is separated off.

Organic substances which are formed or added and are present in the solution of the fermentation broth can be retained or separated by suitable processes. These organic substances include organic by-products that are optionally produced, in addition to the desired amino acid or metabolite, and optionally discharged by the microorganisms employed in the fermentation. These include L-amino acids chosen from the group consisting of L-lysine, L-valine, L-threonine, L-alanine, L-methionine, L-isoleucine, or L-tryptophan. They include vitamins chosen from the group consisting of vitamin B1 (thiamine), vitamin B2 (riboflavin), vitamin B5 (pantothenic acid), vitamin B6 (pyridoxine), vitamin B12 (cyanocobalamin), nicotinic acid/nicotinamide and vitamin E (tocopherol). They also include organic acids that carry one to three carboxyl groups, such as, acetic acid, lactic acid, citric acid, malic acid or fumaric acid. Finally, they also include sugars, for example, trehalose. These compounds are optionally desired if they improve the nutritional value of the product.

These organic substances, including L- and/or D-amino acid and/or the racemic mixture D,L-amino acid, can also be added, depending on requirements, as a concentrate or pure substance in solid or liquid form during a suitable process step. These organic substances mentioned can be added individually or as mixtures to the resulting or concentrated fermentation broth, or also during the drying or granulation process. It is likewise possible to add an organic substance or a mixture of several organic substances to the fermentation broth and a further organic substance or a further mixture of several organic substances during a later process step, for example granulation. The product described above can be used as a feed additive, i.e. feed additive, for animal nutrition. For methods of preparing amino acids for use as feed additives, see, e.g., WO 02/18613, the contents of which are herein incorporated by reference.

Variant Polypeptides

As described in greater detail below, variant polypeptides, for example, polypeptides having one or more amino acid alterations that reduce or eliminate feedback inhibition are useful for the production of amino acids and other metabolites. Examples of variant polypeptides are described below.

6-Phosphogluconate dehydrogenase (gnd) 6-phosphogluconate dehydrogenase catalyzes the oxidation and decarboxylation of 6-phosphogluconate to D-ribulose-5-phosphate. This reaction also regenerates NADPH, which is required for a variety of reductive biosynthesis, including the formation of aspartate-derived amino acids. Gnd is feedback-inhibited by allosterically inhibited by intracellular metabolites such as ATP. Examples of Gnd point mutations effective for decreasing feedback are listed for a number of bacterial species, in Table 10.

TABLE 10
Amino Acid Substitutions in the 6-phosphogluconate dehydrogenase gene
(gnd) that alleviate allosteric regulation

| Organism | Amino Acid Substitution |
|---|---|
| C. glutamicum | S361→ F* |
| E. coli | S344→ F |
| S. coelicolor | S348→ F |
| E. chrysanthemi | S344→ F |
| M. smegmatis | S355→ F |

*described in Ohnishi et al. FEMS Microbiology Letters 242:265.

Homoserine Dehydrogenase (Hom)

Targeted amino acid substitutions can be generated either to decrease, but not eliminate, Hom activity or to relieve Hom from feedback inhibition by threonine. Mutations that result in decreased Hom activity are referred to as “leaky” Hom mutations. In the C. glutamicum homoserine dehydrogenase, amino acid residues have been identified that can be mutated to either enhance or decrease Hom activity. Several of these specific amino acids are well-conserved in Hom proteins in other Actinomycetes (see Table 11).

TABLE 11
Amino acid substitutions that result in either “leaky” Hom alleles or
Hom proteins relieved of feedback inhibition by threonine.

| | Corresponding amino acid residue from | | |
|---|---|---|---|
| | heterologous homoserine dehydrogenase | | |
| C. glutamicum residue | M. smegmatis | S. coelicolor | T. fusca |
| Leaky Hom alleles | | | |
| L23F | V10 | L10 | L192 |
| V59A | V46 | V46 | V228 |
| V104I | I90 | I91 | I274 |
| Deregulated Hom | | | |
| alleles | | | |
| G378E | G364 | G362 | G545 |
| K428 truncation | N/a | R412 truncation | R595 |
| | | | truncation |
| hom^{dr}* | N/a | R412 (delete bp | R595 |
| | | 1937 → frameshift | (delete bp |
| | | mutation) | 1785 → |
| | | | frameshift |
| | | | mutation) |

*The hom^{dr} mutation is described on page 11 of WO 93/09225. This mutation is a single base pair deletion at 1964 bp that disrupts the hom^{dr} reading frame at codon 429. This results in a frame shift mutation that induces approximately ten amino acid changes and a premature termination, or truncation, i.e., deletion of approximately the last seven amino acid residues of the polypeptide.

It is believed that this single base deletion in the carboxy terminus of the hom dr gene radically alters the protein sequence of the carboxyl terminus of the enzyme, changing its conformation in such a way that the interaction of threonine with a binding site is prevented.

Aspartokinase (lysC)

Lysine analogs (e.g. S-(2-aminoethyl)cysteine (AEC)) or high concentrations of lysine (and/or threonine) can be used to identify strains with enhanced production of lysine. A significant portion of the known lysine-resistant strains from both C. glutamicum and E. coli contain mutations at the lysC locus. Importantly, specific amino acid substitutions that confer increased resistance to AEC have been identified, and these substitutions map to well-conserved residues. Specific amino acid substitutions that result in increased lysine productivity, at least in wild-type strains, include, but are not limited to, those listed in Table 12. In many instances, several useful substitutions have been identified at a particular residue. Furthermore, in various examples, strains have been identified that contain more than one lysC mutation. Sequence alignment confirms that the residues previously associated with feedback-resistance (i.e. AEC-resistance) are conserved in a variety of aspartokinase proteins from distantly related bacteria.

TABLE 12
Amino Acid Substitutions That Release
Aspartokinase Feedback Inhibition.

| Organism | Amino Acid Substitution |
|---|---|
| Corynebacterium glutamicum (corresponding | Ala 279 [symbol] Pro |
| amino acids can be identified in | |
| related coryneform bacteria as well) | |
| Corynebacterium glutamicum (corresponding | Ser 301 [symbol] Tyr |
| amino acids can be identified in | |
| related coryneform bacteria as well) | |
| Corynebacterium glutamicum (corresponding | Thr 311 [symbol] Ile |
| amino acids can be identified in | |
| related coryneform bacteria as well) | |
| Corynebacterium glutamicum (corresponding | Gly 345 [symbol] Asp |
| amino acids can be identified in | |
| related coryneform bacteria as well) | |
| Escherichia coli (many substitutions identified | Gly 323 [symbol] Asp |
| between amino acids 318-325 and 345-352) | |
| Escherichia coli (many substitutions identified | Leu 325 [symbol] Phe |
| between amino acids 318-325 and 345-352) | |
| Escherichia coli (many substitutions identified | Ser 345 [symbol] Ile |
| between amino acids 318-325 and 345-352) | |
| Escherichia coli (many substitutions identified | Val 347 [symbol] Met |
| between amino acids 318-325 and 345-352) | |

Standard site-directed mutagenesis techniques can be used to construct aspartokinase variants that are not subject to allosteric regulation. After cloning PCR-amplified lysC or aspartokinase III genes into appropriate shuttle vectors, oligonucleotide-mediated site-directed mutagenesis is use to provide modified alleles that encode substitutions. Vectors containing either wild-type genes or modified alleles can be be transformed into C. glutamicum alongside control vectors. The resulting transformants can be screened, for example, for lysine productivity, increased resistance to AEC, relative cross-feeding of lysine auxotrophs, or other methods known to those skilled in the art to identify the mutant alleles of most interest. Assays to measure lysine productivity and/or enzyme activity can be used to confirm the screening results and select useful mutant alleles. Techniques such as high pressure liquid chromatography (HPLC) and HPLC-mass spectrometry (MS) assays to quantify levels of members of the aspartic acid family of amino acids and related metabolites are known to those skilled in the art.

Methods for random generating amino acid substitutions within the lysC coding sequence, through methods such as mutagenenic PCR, can be used. These methods are familiar to those skilled in the art; for example, PCR can be performed using the GeneMorph PCR mutagenesis kit (Stratagene, La Jolla, Calif.) according to manufacturer's instructions to achieve medium and high range mutation frequencies.

Evaluation of the heterologous enzymes can be carried out in the presence of the proteins that are endogenous to the host strain. In certain instances, it will be helpful to have reagents to specifically assess the functionality of the heterologous biosynthetic proteins. Phenotypic assays for AEC resistance or enzyme assays can be used to confirm function of wild-type and modified variants of heterologous aspartokinases. The function of cloned heterologous genes can be confirmed by complementation of genetically characterized mutants of E. coli or C. glutamicum. Many of the E. coli strains are publicly available from the E. coli Genetic Stock Center (http://cgsc.biology.yale.edu/top.html). C. glutamicum mutants have also been described.

Methionine Adenosyltransferase

Targeted amino acid substitutions can be generated to decrease, but not eliminate, MetK activity. Mutations that result in decreased MetK activity are referred to as “leaky” MetK mutations. In the C. glutamicum and E. coli MetK polypeptides, amino acid residues have been identified that can be mutated to decrease MetK activity. These specific amino acids are well-conserved in MetK proteins in other Actinomycetes and E. chrysanthemi (see Table 13).

TABLE13
Amino acid substitutions that result in “leaky” MetK alleles.

| Leaky MetK | Corresponding amino acid | | | | |
|---|---|---|---|---|---|
| allele | residue from heterologous MetK | | | | |
| C. glutamicum | M. | E. | S. | | |
| residue | smegmatis | chrysanthemi | coelicolor | T. fusca | E. coli |
| V200E | V196E | V185E | V195E | V195E | V185E |

### EXAMPLES

Described below are methods for constructing vectors for expressing the polypeptides described herein as well as methods for construction variant polypeptides.

### Example 1

### Construction of Vectors for Expression of Genes for Enhancing Production of Aspartate-Derived Amino Acids

Plasmids were generated for expression of genes relevant to the production of aspartate-derived amino acids. Many of the target genes are shown in FIG. 1. These plasmids, which may either replicate autonomously or integrate into the host C. glutamicum chromosome, were introduced into strains of corynebacteria by electroporation as described (see Follettie, M. T., et al. J. Bacteriol. 167:695-702, 1993). All plasmids contain the kanR gene that confers resistance to the antibiotic kanamycin. Transformants were selected on media containing kanamycin (25 mg/L).

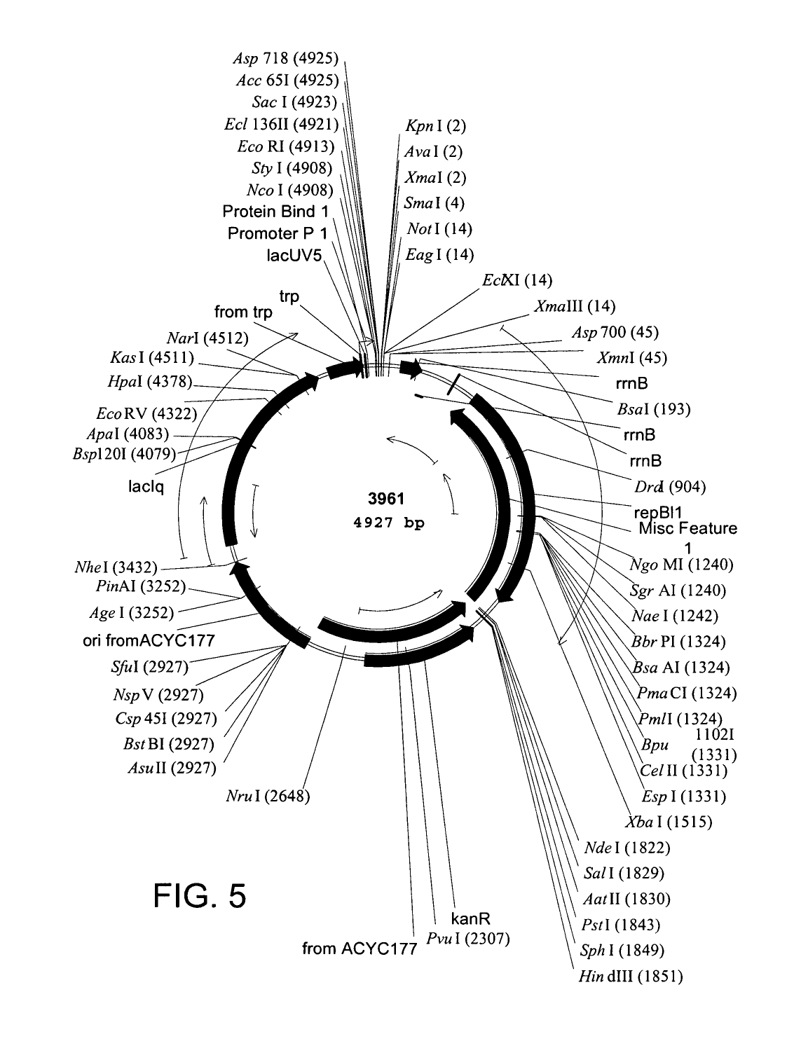

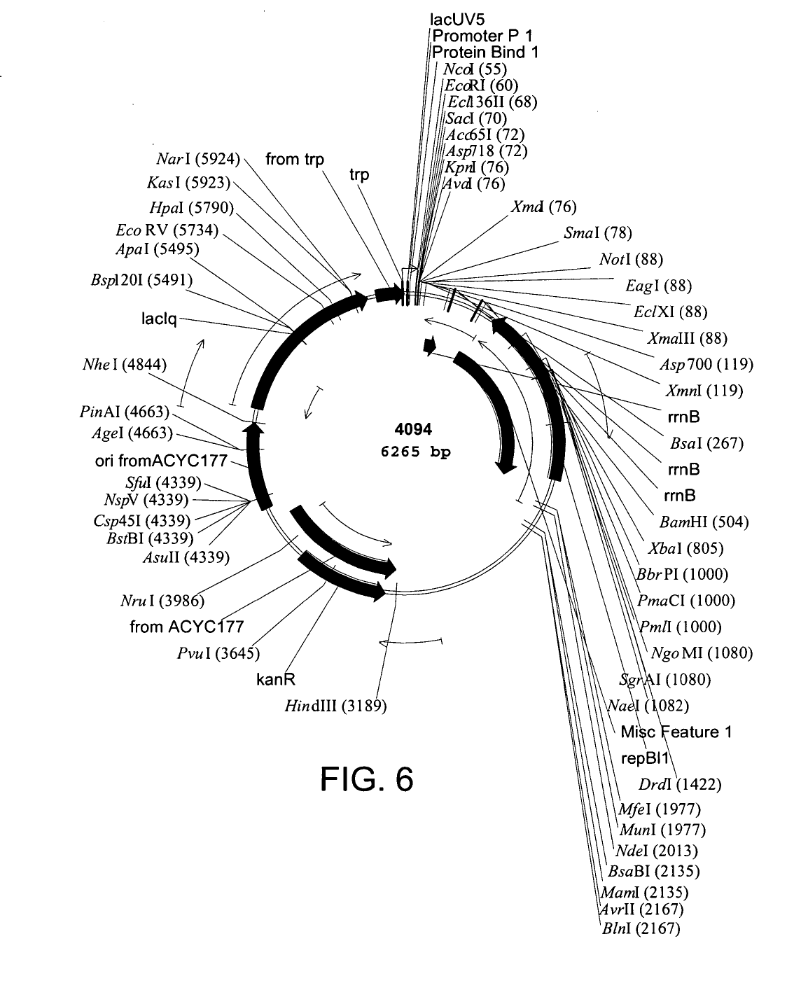

For expression from episomal plasmids, vectors were constructed using derivatives of the cryptic C. glutamicum low-copy pBL1 plasmid (see Santamaria et al. J. Gen. Microbiol. 130:2237-2246, 1984). Episomal plasmids contain sequences that encode a replicase, which enables replication of the plasmid within C. glutamicum; therefore, these plasmids can be propagated without integration into the chromosome. Plasmids MB3961 and MB4094 were the vector backbones used to construct episomal expression plasmids described herein (see FIGS. 5 and 6). Plasmid MB4094 contains an improved origin of replication, relative to MB3961, for use in corynebacteria; therefore, this backbone was used for most studies. Both MB3961 and MB4094 contain regulatory sequences from pTrc99A (see Amann et al., Gene 69:301-315, 1988). The 3′ portion of the lacIq-trc IPTG-inducible promoter cassette resides within the polylinker in such a way that genes of interest can be inserted as fragments containing NcoI-NotI compatible overhangs, with the NcoI site adjacent to the start site of the gene of interest (additional polylinker sites such as KpnI can also be used instead of the NotI site). In addition, useful promoters such as a modified trc promoter (trcRBS) and the C. glutamicum gpd, rplM, and rpsJ promoters can be inserted into the MB3961 and MB4094 backbones on convenient restriction fragments, including NheI-NcoI fragments. The trcRBS promoter contains a modified ribosomal-binding site that was shown to enhance levels of expressed proteins. The sequences of promoters employed in these studies for expression of genes are found in Table 14.

TABLE 14

Promoters used to control expression of genes in
corynebacteria.

| | | SEQ ID |
|---|---|---|
| Promoter | Sequence | NO: |
| LacIq-trc | ctagctacgttgacaccatcgaatggtgcaaa | 297 |
| | acctttcgcggtatggcatgatagcgcccgga | |
| | agagagtcaattcagggtggtgaatgtgaaac | |
| | cagtaacgttatacgatgtcgcagagtatgcc | |
| | ggtgtctcttatcagaccgtttcccgcgtggt | |
| | gaaccaggccagccacgtttctgcgaaaacgc | |
| | gggaaaaagtggaagcggcgatggcggagctg | |
| | aattacattcccaaccgcgtggcacaacaact | |
| | ggcgggcaaacagtcgttgctgattggcgttg | |
| | ccacctccagtctggccctgcacgcgccgtcg | |
| | caaattgtcgcggcgattaaatctcgcgccga | |
| | tcaactgggtgccagcgtggtggtgtcgatgg | |
| | tagaacgaagcggcgtcgaagcctgtaaagcg | |
| | gcggtgcacaatcttctcgcgcaacgcgtcag | |
| | tgggctgatcattaactatccgctggatgacc | |
| | aggatgccattgctgtggaagctgcctgcact | |
| | aatgttccggcgttatttcttgatgtctctga | |
| | ccagacacccatcaacagtattattttctccc | |
| | atgaagacggtacgcgactgggcgtggagcat | |
| | ctggtcgcattgggtcaccagcaaatcgcgct | |
| | gttagcgggcccattaagttctgtctcggcgc | |
| | gtctgcgtctggctggctggcataaatatctc | |
| | actcgcaatcaaattcagccgatagcggaacg | |
| | ggaaggcgactggagtgccatgtccggttttc | |
| | aacaaaccatgcaaatgctgaatgagggcatc | |
| | gttcccactgcgatgctggttgccaacgatca | |
| | gatggcgctgggcgcaatgcgcgccattaccg | |
| | agtccgggctgcgcgttggtgcggatatctcg | |
| | gtagtgggatacgacgataccgaagacagctc | |
| | atgttatatcccgccgttaaccaccatcaaac | |
| | aggattttcgcctgctggggcaaaccagcgtg | |
| | gaccgcttgctgcaactctctcagggccaggc | |
| | ggtgaagggcaatcagctgttgcccgtctcac | |
| | tggtgaaaagaaaaaccaccctggcgcccaat | |
| | acgcaaaccgcctctccccgcgcgttggccga | |
| | ttcattaatgcagctggcacgacaggtttccc | |
| | gactggaaagcgggcagtgagcgcaacgcaat | |
| | taatgtgagttagcgcgaattgatctggtttg | |
| | acagcttatcatcgactgcacggtgcaccaat | |
| | gcttctggcgtcaggcagccatcggaagctgt | |
| | ggtatggctgtgcaggtcgtaaatcactgcat | |
| | aattcgtgtcgctcaaggcgcactcccgttct | |
| | ggataatgttttttgcgccgacatcataacgg | |
| | ttctggcaaatattctgaaatgagctgttgac | |
| | aattaatcatccggctcgtataatgtgtggaa | |
| | ttgtgagcggataacaatttcacacaggaaac | |
| | agac | |
| LacIq- | ctagctacgttgacaccatcgaatggtgcaaa | 298 |
| trcRBS | acctttcgcggtatggcatgatagcgcccgga | |
| | agagagtcaattcagggtggtgaatgtgaaac | |
| | cagtaacgttatacgatgtcgcagagtatgcc | |
| | ggtgtctcttatcagaccgtttcccgcgtggt | |
| | gaaccaggccagccacgtttctgcgaaaacgc | |
| | gggaaaaagtggaagcggcgatggcggagctg | |
| | aattacattcccaaccgcgtggcacaacaact | |
| | ggcgggcaaacagtcgttgctgattggcgttg | |
| | ccacctccagtctggccctgcacgcgccgtcg | |
| | caaattgtcgcggcgattaaatctcgcgccga | |
| | tcaactgggtgccagcgtggtggtgtcgatgg | |
| | tagaacgaagcggcgtcgaagcctgtaaagcg | |
| | gcggtgcacaatcttctcgcgcaacgcgtcag | |
| | tgggctgatcattaactatccgctggatgacc | |
| | aggatgccattgctgtggaagctgcctgcact | |
| | aatgttccggcgttatttcttgatgtctctga | |
| | ccagacacccatcaacagtattattttctccc | |
| | atgaagacggtacgcgactgggcttggagcat | |
| | ctggtcgcattgggtcaccagcaaatcgcgct | |
| | gttagcgggcccattaagttctgtctcggcgc | |
| | gtctgcgtctggctggctggcataaatatctc | |
| | actcgcaatcaaattcagccgatagcggaacg | |
| | ggaaggcgactggagtgccatgtccggttttc | |
| | aacaaaccatgcaaatgctgaatgagggcatc | |
| | gttcccactgcgatgctggttgccaacgatca | |
| | gatggcgctgggcgcaatgcgcgccattaccg | |
| | agtccgggctgcgcgttggtgcggatatctcg | |
| | gtagtgggatacgacgataccgaagacagctc | |
| | atgttatatcccgccgttaaccaccatcaaac | |
| | aggattttcgcctgctggggcaaaccagcgtg | |
| | gaccgcttgctgcaactctctcagggccaggc | |
| | ggtgaagggcaatcagctgttgcccgtctcac | |
| | tggtgaaaagaaaaaccaccctggcgcccaat | |
| | acgcaaaccgcctctccccgcgcgttggccga | |
| | ttcattaatgcagctggcacgacaggtttccc | |
| | gactggaaagcgggcagtgagcgcaacgcaat | |
| | taatgtgagttagcgcgaattgatctggtttg | |
| | acagcttatcatcgactgcacggtgcaccaat | |
| | gcttctggcgtcaggcagccatcggaagctgt | |
| | ggtatggctgtgcaggtCgtaaatcactgcat | |
| | aattcgtgtcgctcaaggcgcactcccgttct | |
| | ggataatgttttttgcgccgacatcataacgg | |
| | ttctggcaaatattctgaaatgagctgttgac | |
| | aattaatcatccggctcgtataatgtgtggaa | |
| | ttgtgagcggataacaatttcacacaggaaac | |
| | agagaattcaaaggaggacaac | |
| C. | ctagcctaaaaacgaccgagcctattgggatt | 299 |
| glutamicum | accattgaagccagtgtgagttgcatcacatt | |
| gpd | ggcttcaaatctgagactttaatttgtggatt | |
| | cacgggggtgtaatgtagttcataattaaccc | |
| | cattcgggggagcagatcgtagtgcgaacgat | |
| | ttcaggttcgttccctgcaaaaactatttagc | |
| | gcaagtgttggaaatgcccccgtttggggtca | |
| | atgtccatttttgaatgtgtctgtatgatttt | |
| | gcatctgctgcgaaatctttgtttccccgcta | |
| | aagttgaggacaggttgacacggagttgactc | |
| | gacgaattatccaatgtgagtaggtttggtgc | |
| | gtgagttggaaaaattcgccatactcgccctt | |
| | gggttctgtcagctcaagaattcttgagtgac | |
| | cgatgctctgattgacctaactgcttgacaca | |
| | ttgcatttcctacaatctttagaggagacaca | |
| | ac | |
| C. | ctagcggggttgctgcactttttaaaaaggca | 300 |
| glutamicum | aaaaatagcgaaaacacaccccaggtttttcc | |
| rplM | cgtaaccccgctaggctatgcaatttcggttt | |
| | aacccagtttttcaaagaaggtcactagcttt | |
| | tccgctggtcaccttctttttggtttttcaac | |
| | gcagagatagtacactttactctttgtgtgtg | |
| | gagtcaaacctcccctttaaggggtgcgcttg | |
| | gacagcaggacaaattcgggtcaccaccggcc | |
| | gccgaatttagcttccttccgaacatattcct | |
| | ggctggcagttctagaccgactaattcaagga | |
| | gtcattc | |
| C. | ctagctatttcagtgcggggcagtgaaagtaa | 301 |
| glutamicum | aaacgcaactttcttacagaacagggttgtct | |
| rpsJ | ttcagacgactatgtggttaactacttgggct | |
| | gctttaacacggcgtgaattaaccatgccagt | |
| | tggtaaggcaaacatgacaccttcaattggag | |
| | tcgaggcgcatgaaaatgcacttcaacttcag | |
| | ggggtatccactgaagccgggtgactggtgaa | |
| | ggcggaaccggagaaggggcatggcaaataaa | |
| | cagcggcagttacgttagggcctagatcacgc | |
| | attttggtcccttccgatttccctgacttcat | |
| | tgttgggttcatcgtggagcgttttatttgta | |
| | cagcgcccgtgatccaatgtcagaagcatttg | |
| | acaggtcaggttaaacactggcgttgcgcccg | |
| | agccccaagcccggacaacgttatagagaaag | |
| | aatgaagcgaattcccaccgcttttccaaaat | |
| | ggaagatgtgggacgagcgaggaagaggataa | |
| | gc | |

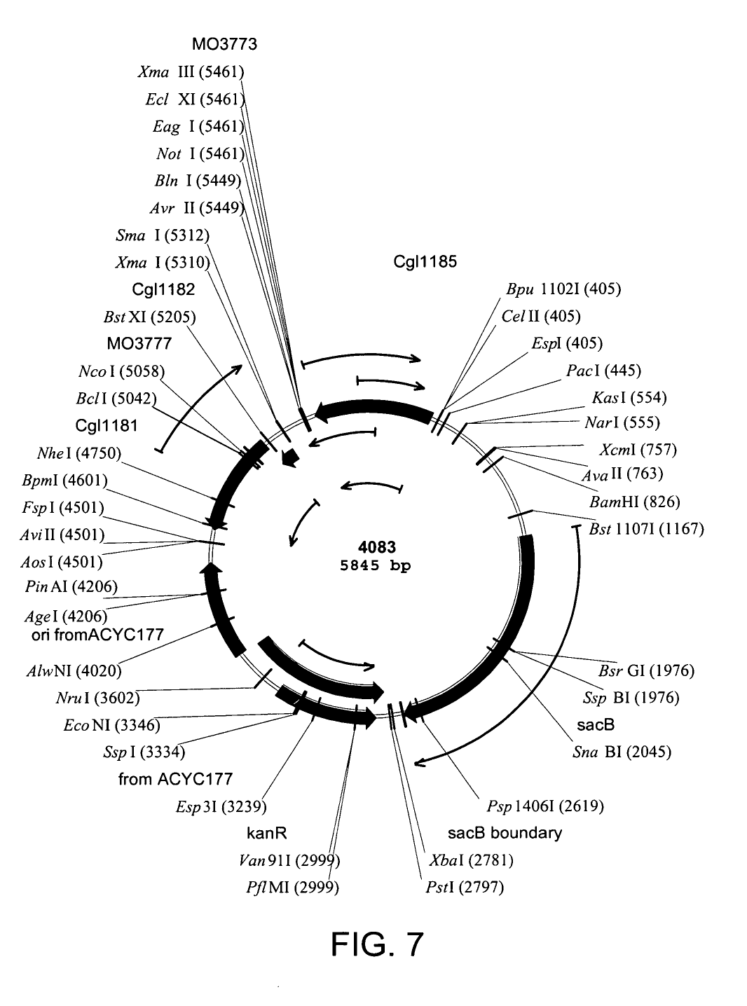

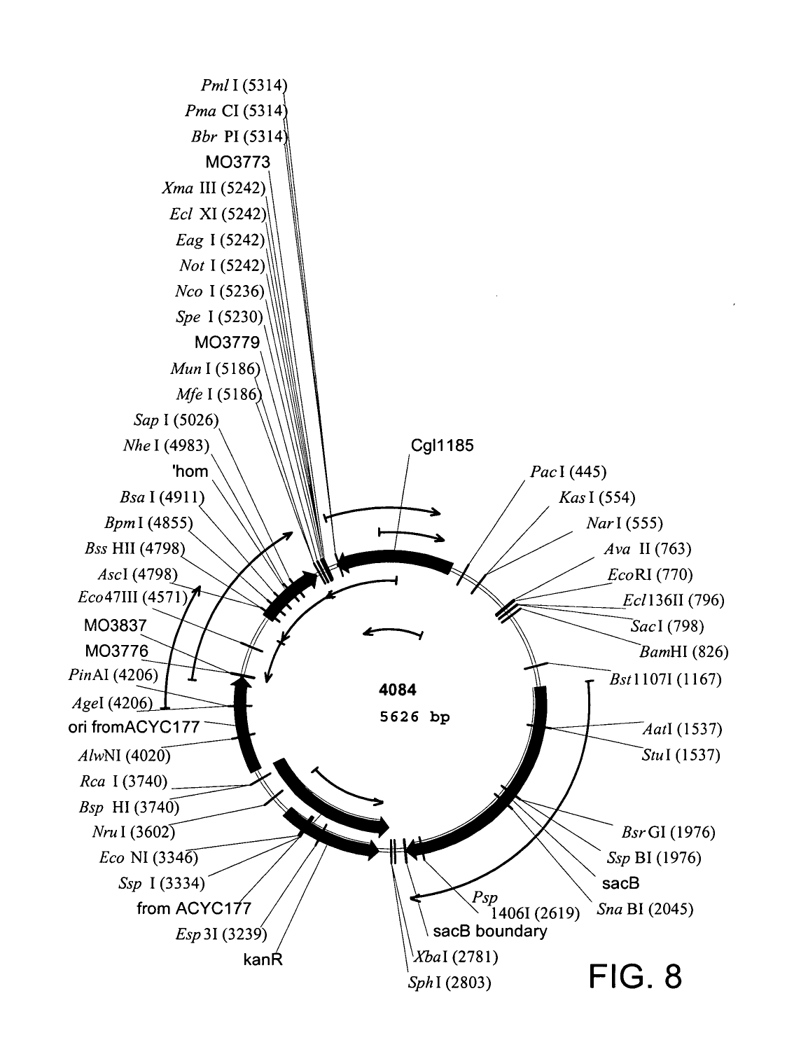

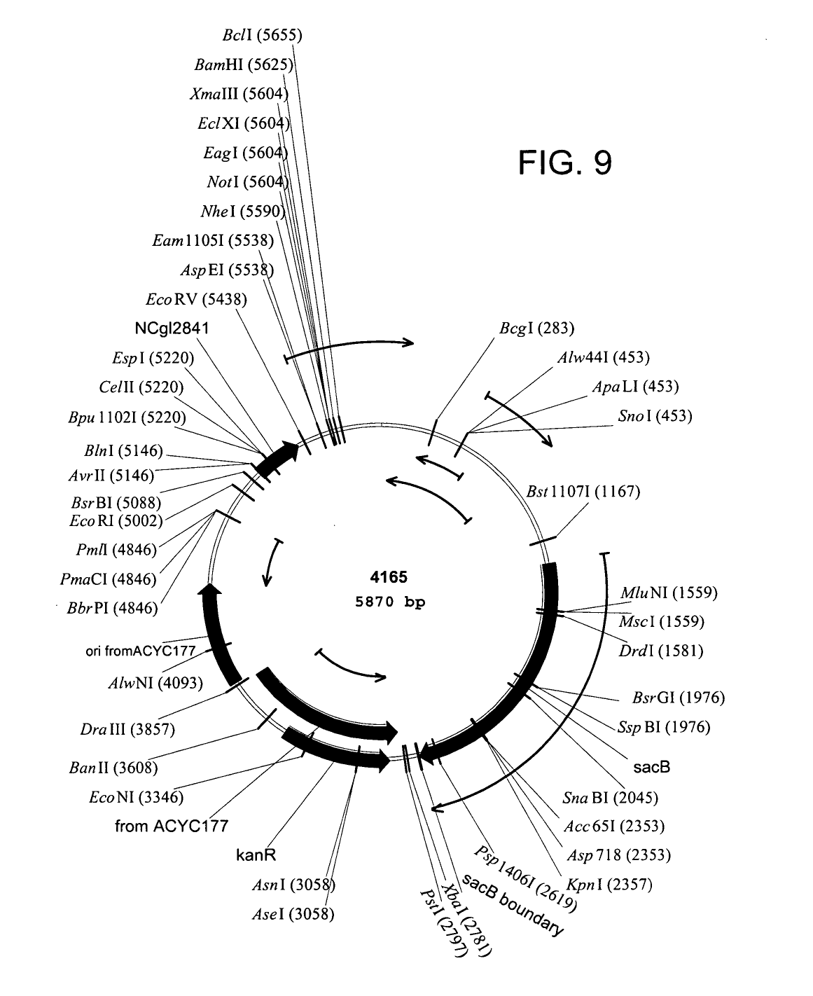

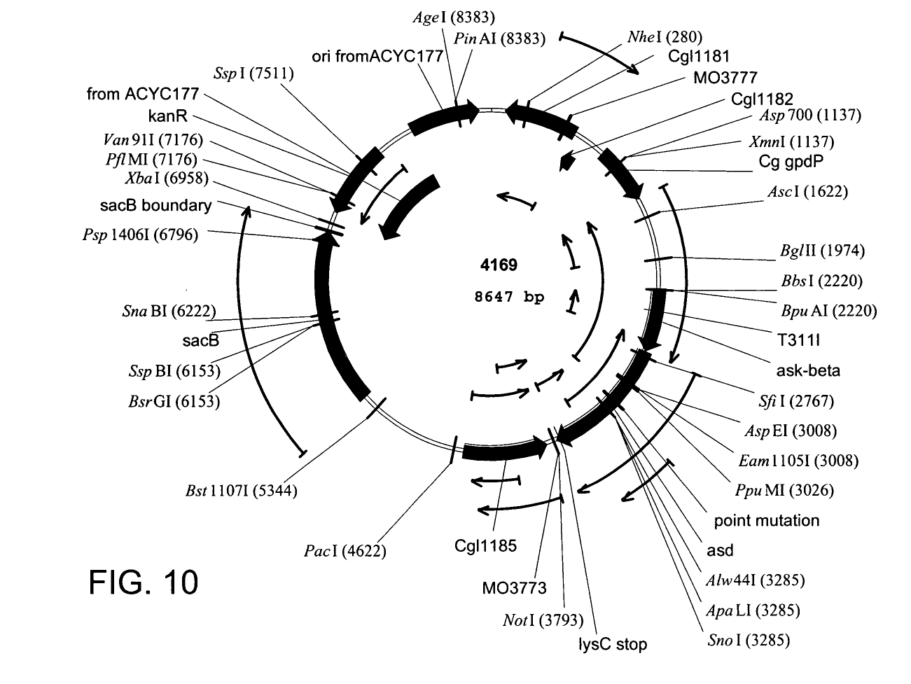

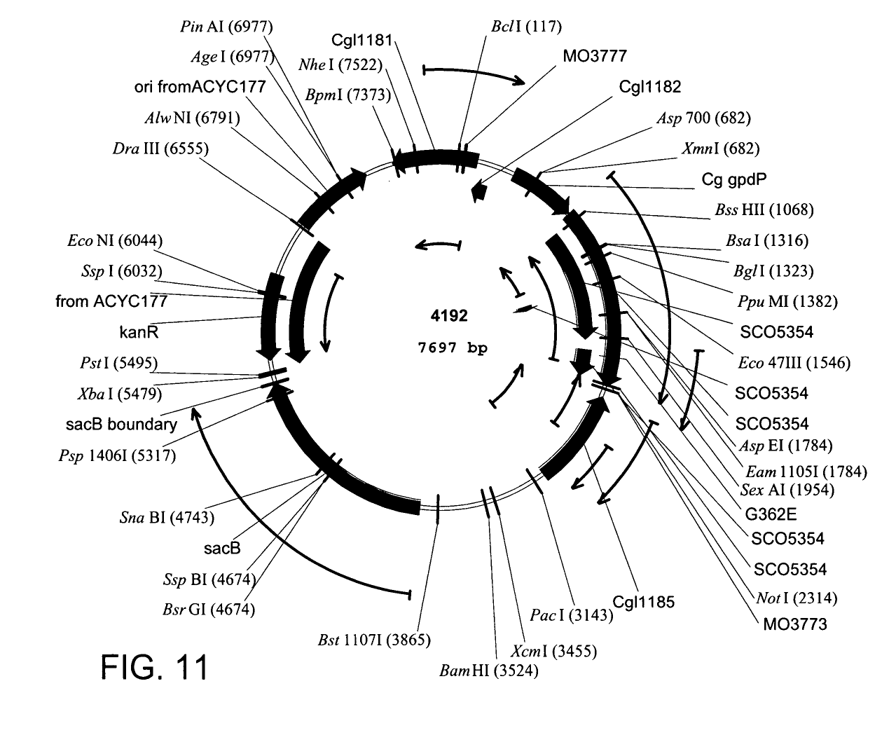

Plasmids were also designed to inactivate native C. glutamicum genes by gene deletion. In some instances, these constructs both delete native genes and insert heterologous genes into the host chromosome at the locus of the deletion event. Table 14 lists the endogenous gene that was deleted and the heterologous genes that were introduced, if any. Deletion plasmids contain nucleotide sequences homologous to regions upstream and downstream of the gene that is the target for the deletion event; in some instances these sequences include small amounts of coding sequence of the gene that is to be inactivated. These flanking sequences are used to facilitate homologous recombination. Single cross-over events target the plasmid into the host chromosome at sites upstream or downstream of the gene to be deleted. Deletion plasmids also contain the sacB gene, encoding the levansucrase gene from Bacillus subtilis. Transformants containing integrated plasmids were streaked to BHI medium lacking kanamycin. After day, colonies were streaked onto BHI medium containing 10% sucrose. This protocol selects for strains in which the sacB gene has been excised, since it polymerizes sucrose to form levan that is toxic to C. glutamicum (see Jager, W., et al. J. Bacteriol. 174:5462-5465, 1992). During growth of transformants upon medium containing sucrose, sacB allows for positive selection for recombination events, resulting in either a clean deletion event or removal of all portions of the integrating plasmid except for the cassette that regulates the inducible expression of a particular gene of interest (see Jager, W., et al. J. Bacteriol. 174:5462-5465, 1992). PCR, together with growth on diagnostic media, was used to verify that expected recombination events have occurred in sucrose-resistant colonies. FIGS. 7-14A display deletion plasmids described herein.

TABLE 15
Plasmids used for deletion of C. glutamicum genes, sometimes in
conjunction with insertion of expression cassettes.

| Plasmid | Native gene(s) deleted | Element inserted at locus |
|---|---|---|
| MB4083 | hom-thrB | None |
| MB4084 | thrB | None |
| MB4165 | mcbR | None |
| MB4169 | hom-thrB | gpd-M. smegmatis |
| | | lysC(T311I)-asd |
| MB4192 | hom-thrB | gpd-S. coelicolor |
| | | hom (G362E) |
| MB4276 | pck | gpd-M. smegmatis |
| | | lysC(T311I)-asd |
| MB4286 | mcbR | trcRBS-T. fusca metA |
| MB4287 | mcbR | trcRBS-C. glutamicum metA |
| | | (K233A)-metB |

### Example 2

### Isolation of Genes for Enhancing Production of Aspartate-Derived Amino Acids

Wild-type alleles of aspartokinase alpha (lysC-alpha) and beta (lysC-beta) and aspartate semialdehyde dehydrogenase (asd) from Mycobacterium smegmatis (homologs of lysC/asd in Corynebacterium glutamicum); genes encoding aspartokinase-asd (lysC-asd), dapA, and hom from Streptomyces coelicolor; metA and metYA from Thermobifida fusca; and dapA and ppc from Erwinia chrysanthemi were obtained by PCR amplification using genomic DNA isolated from each organism. In addition, in some cases the corresponding wild-type allele for each gene was isolated from C. glutamicum. Amplicons were subsequently cloned into pBluescriptSK II⁻ for sequence verification; in particular instances, site-directed mutagenesis to create the activated alleles was also performed in these vectors. Genomic DNA was isolated from M. smegmatis grown in BHI medium for 72 h at 37° C. using QIAGEN Genomic-tips according to the recommendations of the manufacturer kits (Qiagen, Valencia, Calif.). For the isolation of genomic DNA from S. coelicolor, the Salting Out Procedure (as described in Practical Streptomyces Genetics, pp. 169-170, Kieser, T., et. al., John Innes Foundation, Norwich, England 2000) was used on cells grown in TYE media (ATCC medium 1877 ISP Medium 1) for 7 days at 25° C.

To isolate genomic DNA from T. fusca, cells were grown in TYG media (ATCC medium 741) for 5 days at 50° C. The 100 ml culture was spun down (5000 rpm for 10 min at 4° C.) and washed twice with 40 ml 10 mM Tris, 20 mM EDTA pH 8.0. The cell pellet was brought up in a final volume of 40 ml of 10 mMTris, 20 mM EDTA pH 8.0. This suspension was passed through a Microfluidizer (Microfluidics Corporation, Newton Mass.) for 10 cycles and collected. The apparatus was rinsed with an additional 20 ml of buffer and collected. The final volume of lysed cells was 60 ml. DNA was precipitated from the suspension of lysed cells by isopropanol precipitation, and the pellet was resuspended in 2 ml TE pH 8.0. The sample was extracted with phenol/chloroform and the DNA precipitated once again with isopropanol. To isolate DNA from E. chrysanthemi, genomic DNA was prepared as described for E. coli (Qiagen genomic protocol) using a Genomic Tip 500/G.

For PCR amplification of the M. smegmatis lysC-asd operon, primers were designed according to sequence upstream of the lysC gene and sequence near the stop of asd. The upstream primer is 5′-CCGTGAGCTGCTCGGATGTGACG-3′(SEQ ID NO:302), the downstream primer is 5′-TCAGAGGTCGGCGGCCAACAGTTCTGC-3′ (SEQ ID NO:303). The genes were amplified using Pfu Turbo (Stratagene, La Jolla, Calif.) in a reaction mixture containing 10 μl 10× Cloned Pfu buffer, 8 μl dNTP mix (2.5 mM each), 2 μl each primer (20 uM), 1 μl Pfu Turbo, 10 ng genomic DNA and water in a final reaction volume of 100 μl. The reaction conditions were 94° C. for 2 min, followed by 28 cycles of 94° C. for 30 sec, 60° C. for 30 sec, 72° C. for 9 min. The reaction was completed with a final extension at 72° C. for 4 min, and the reaction was then cooled to 4° C. The resulting product was purified by the Qiagen gel extraction protocol followed by blunt end ligation into the Smal site of pBluescript SK II-. Ligations were transformed into E. coli DH5α and selected by blue/white screening. Positive transformants were treated to isolate plasmid DNA by Qiagen methods and sequenced. MB3902 was the resulting plasmid containing the expected insert.

Primer pairs for amplifying S. coelicolor genes are: 5′-ACCGCACTTTCCCGAGTGAC-3′ (SEQ ID NO:304) and 5′-TCATCGTCCGCTCTTCCCCT-3′ (lysC-asd) (SEQ ID NO:305); 5′-ATGGCTCCGACCTCCACTCC-3′ (SEQ ID NO:306) and 5′-CGTGCAGAAGCAGTTGTCGT-3′ (dapA) (SEQ ID NO:307); and 5′-TGAGGTCCGAGGGAGGGAAA-3′ (SEQ ID NO:308) and 5′-TTACTCTCCTTCAACCCGCA-3′ (hom) (SEQ ID NO:309). The primer pair for amplifying the metYA operon from T. fusca is 5′-CATCGACTACGCCCGTGTGA-3′ (SEQ ID NO:310) and 5′-TGGCTGTTCTTCACCGCACC-3′ (SEQ ID NO:311). Primer pairs for amplifying E. chrysanthemi genes are: 5′-TTGACCTGACGCTTATAGCG-3′ (SEQ ID NO:312) and 5′-CCTGTACAAAATGTTGGGAG-3′ (dapA) (SEQ ID NO:313); and 5′-ATGAATGAACAATATTCCGCCA-3′ (SEQ ID NO:314) and 5′-TTAGCCGGTATTGCGCATCC-3′ (ppc) (SEQ ID NO:315).

Amplification of genes was done by similar methods as above or by using the TripleMaster PCR System from Eppendorf (Eppendorf, Hamburg, Germany). Blunt end ligations were performed to clone amplicons into the Smal site of pBluescript SK II-. The resulting plasmids were MB3947 (S. coelicolor lysC-asd), MB3950 (S. coelicolor dapA), MB4066 (S. coelicolor hom), MB4062 (T. fusca metYA), MB3995 (E. chrysanthemi dapA), and MB4077 (E. chrysanthemi ppc). These plasmids were used for sequence verification of inserts and subsequent cloning into expression vectors; a subset of these vectors was also subjected to site-directed mutagenesis to generate deregulated alleles of specific genes.

### Example 3

### Targeted Substitutions to Enhance the Activity of Genes Involved in the Production of Aspartate-Derived Amino Acids

Site-directed mutagenesis was performed on several of the pBluescript SK II-plasmids containing the heterologous genes described in Example 2. Site-directed mutagenesis was performed using the QuikChange Site-Directed Mutagenesis Kit from Stratagene. For heterologous aspartokinase (lysC/ask) genes, substitution mutations were constructed that correspond to the T311I, S301Y, A279P, and G345D amino acid substitutions in the C. glutamicum protein. These substitutions may decrease feedback inhibition by the combination of lysine and threonine. In all instances, the mutated lysC/ask alleles were expressed in an operon with the heterologous asd gene. Oligonucleotides employed to construct M. smegmatis feedback resistant lysC alleles were: 5′-GGCAAGACCGACATCATATTCACGTGTGCGCGTG-3′ (SEQ ID NO:316) and 5′-CACGCGCACACGTGAATATGATGTCGGTCTTGCC-3′ (T311I) (SEQ ID NO:317); 5′-GGTGCTGCAGAACATCTACAAGATCGAGGACGGCAA-3′ (SEQ ID NO:318) and 5′-TTGCCGTCCTCGATCTTGTAGATGTTCTGCAGCACC-3′ (S301Y) (SEQ ID NO:319); 5′-GACGTTCCCGGCTACGCCGCCAAGGTGTTCCGC-3′ (SEQ ID NO:320) and 5′-GCGGAACACCTTGGCGGCGTAGCCGGGAACGTC-3′ (A279P) (SEQ ID NO:321); and 5′-GTACGACGACCACATCGACAAGGTGTCGCTGATCG-3′ (SEQ ID NO:322); and 5′-CGATCAGCGACACCTTGTCGATGTGGTCGTCGTAC-3′ (G345D) (SEQ ID NO:323). Oligonucleotides employed to construct S. coelicolor feedback resistant lysC alleles were:

(SEQ ID NO:324)

5′CGGGCCTGACGGACATCRTCTTCACGCTCCCCAAG-3′
and

(SEQ ID NO:325)

5′-CTTGGGGAGCGTGAAGAYGATGTCCGTCAGGCCCG-3′
(S314I/S314V);
and

(SEQ ID NO:326)

5′-GTCGTGCAGAACGTGTACGCCGCCTCCACGGGC-3′
and

(SEQ ID NO:327)

5′-GCCCGTGGAGGCGGCGTACACGTTCTGCACGAC-3′
(S304Y).

Site-directed mutagenesis can be performed to generate deregulated alleles of additional proteins relevant to the production of aspartate-derived amino acids. For example, mutations can be generated that correspond to the V59A, G378E, or carboxy-terminal truncations of the C. glutamicum hom gene. The Transformer Site-Directed Mutagenesis Kit (BD Biosciences Clontech) was used to generate the S. coelicolor hom (G362E) substitution. Oligonucleotides 5′-GTCGACGCGTCTTAAGGCATGCAAGC-3′(SEQ ID NO:328) and 5′-CGACAAACCGGAAGTGCTCGCCC-3′ (SEQ ID NO:329) were utilized to construct the mutation. Site-directed mutagenesis was also employed to generate specific alleles of the T. fusca and C. glutamicum metA and metY genes (see examples 5 and 6 of the instant specification). Similar strategies can be used to construct deregulated alleles of additional pathway proteins. For example, oligonucleotides 5′-TTCATCGAACAGCGCTCGCACCTGCTGACCGCC-3′ (SEQ ID NO:330) and 5′-GGCGGTCAGCAGGTGCGAGCGCTGTTCGATGAA-3′ (SEQ ID NO:331)can be used to generate a substitution in the S. coelicolor pyc gene that corresponds to the C. glutamicumpyc P458S mutation. Site-directed mutagenesis can also be utilized to introduce substitutions that correspond to deregulated dapA alleles described above.

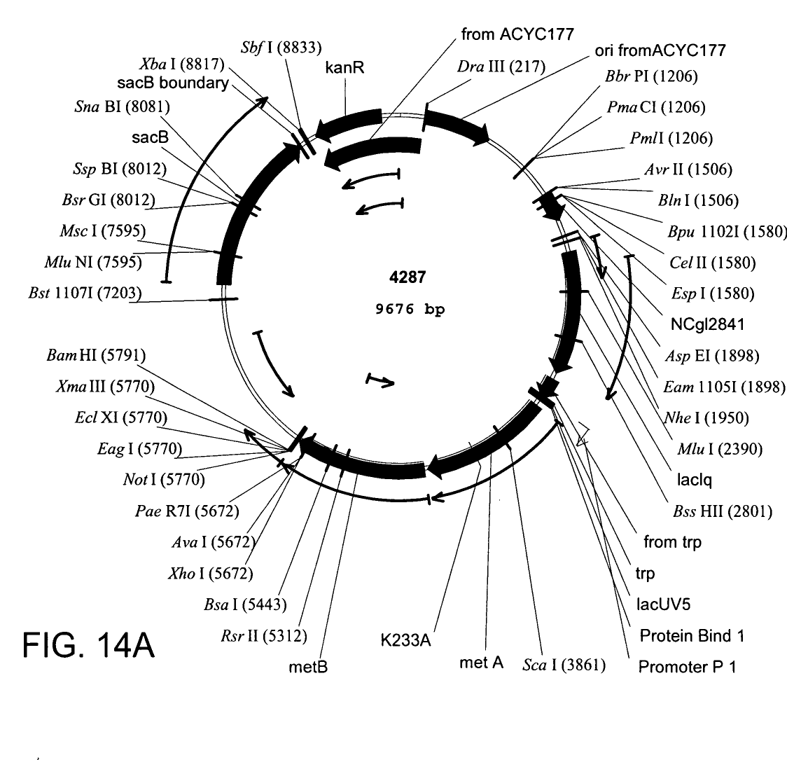

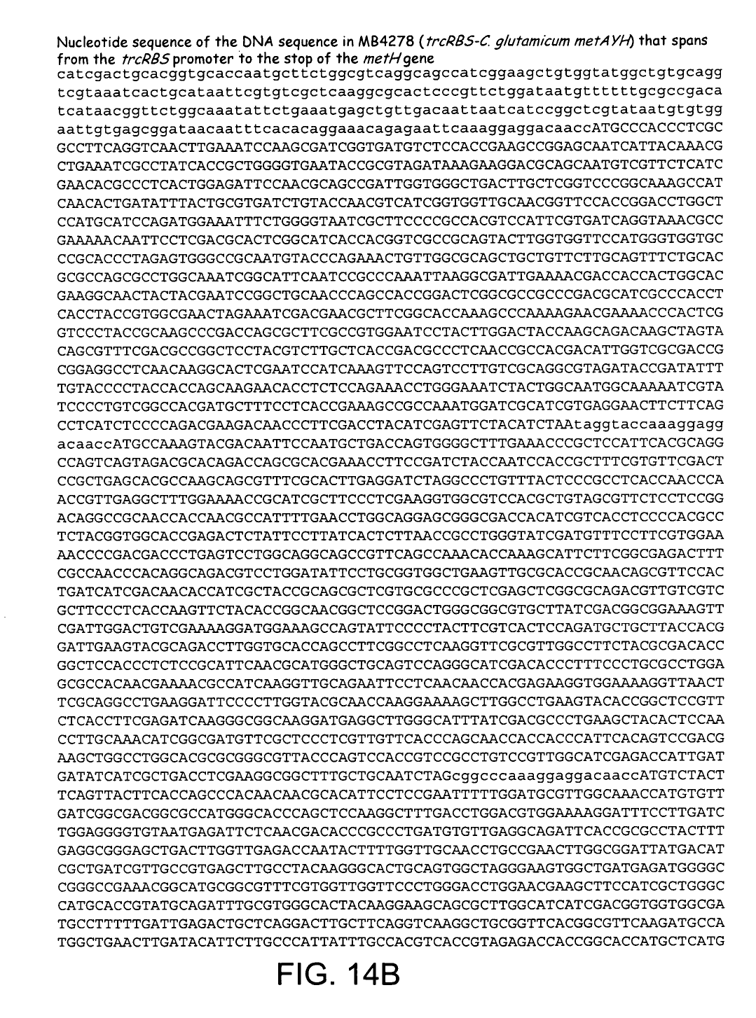

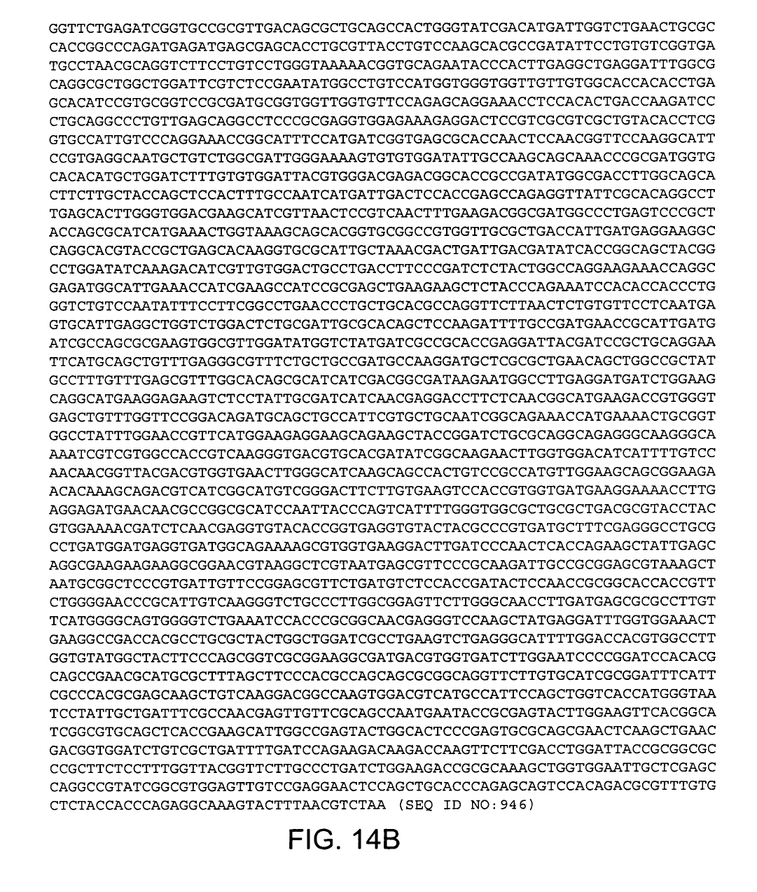

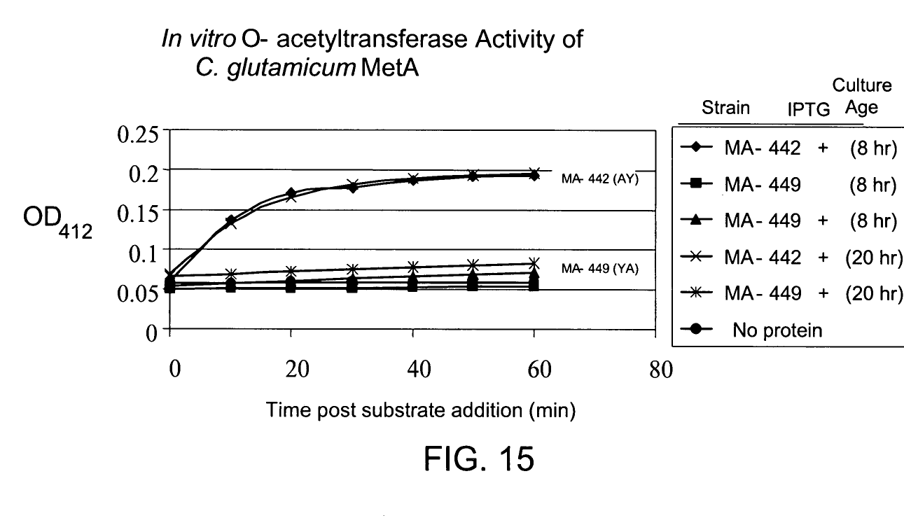

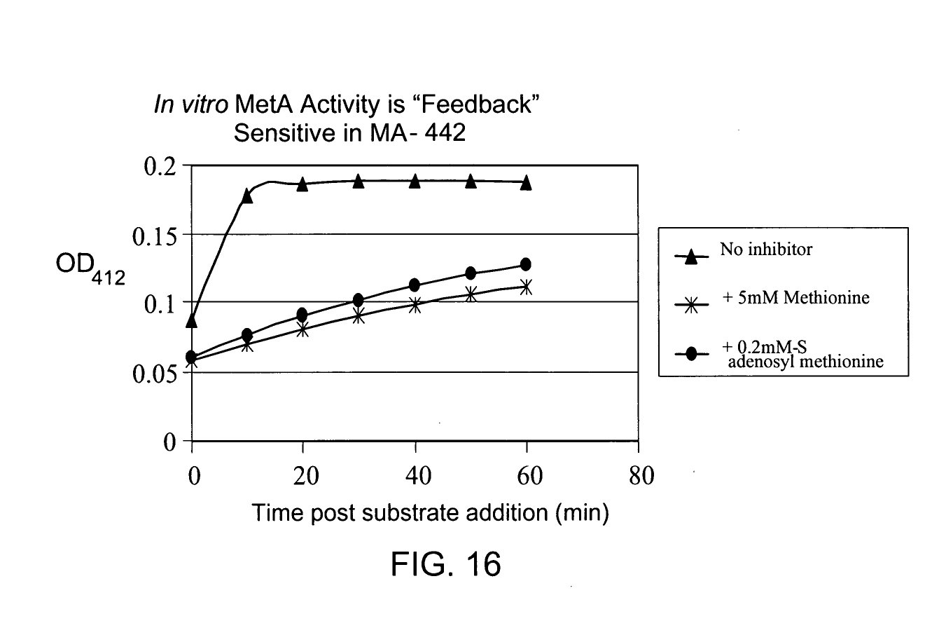

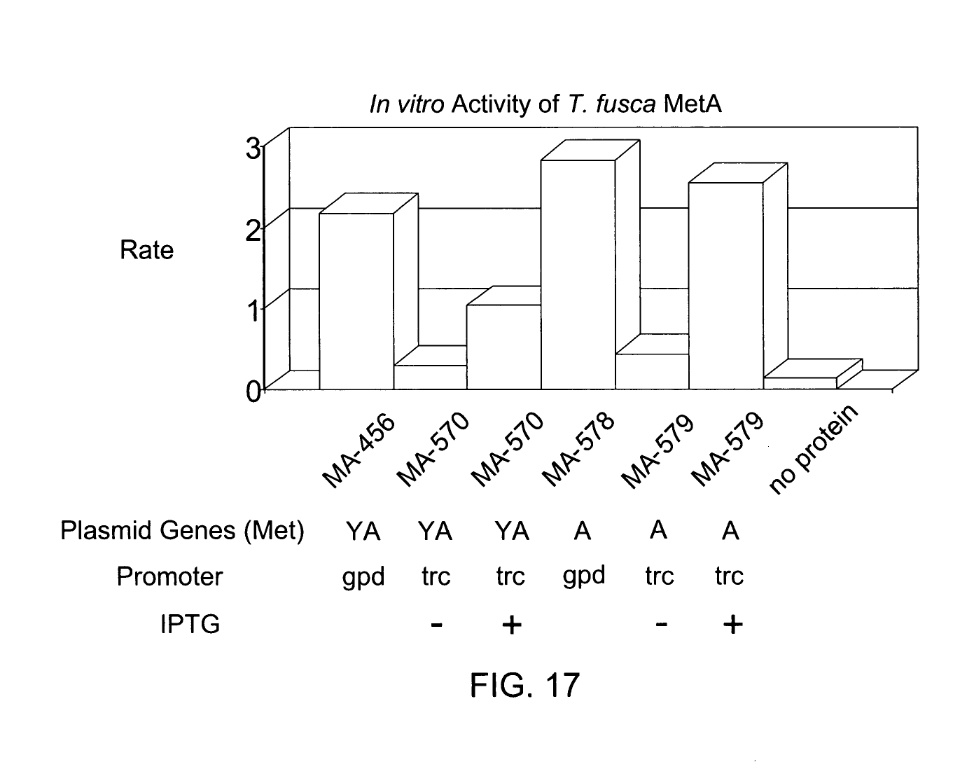

Wild-type and deregulated alleles of heterologous (and C. glutamicum) genes were then cloned into vectors suitable for expression. In general, PCR was employed using oligonucleotides to facilitate cloning of genes as a NcoI-NotI fragment. DNA sequence analysis was performed to verify that mutations were not introduced during rounds of amplification. In some instances, synthetic operons were constructed in order to express two or more genes, heterologous or endogenous, from the same promoter. As an example, plasmid MB4278 was generated to express the C. glutamicum metA, metY, and metH genes from the trcRBS promoter. FIG. 14B displays the DNA sequence in MB4278 that spans from the trcRBS promoter to the stop of the metH gene; the gene order in this construct is metAYH. The open reading frames in FIG. 14B are shown in uppercase. Note that the construct was engineered such that each open reading frame is preceded by an identical stretch of DNA. This conserved sequence serves as a ribosomal-binding sequence that promotes efficient translation of C. glutamicum proteins. Similar intergenic sequences were used to construct additional synthetic operons.

### Example 4

### Isolation of Additional Threonine-Insensitive Mutants of Homoserine Dehydrogenase

The hom gene cloned from S. coelicolor in Example 2 is subjected to error prone PCR using the GeneMorph® Random Mutagenesis kit obtained from Stratagene. Under the conditions specified in this kit, oligonucleotide primers 5′-CACACGAAGACACCATGATGCGTACGCGTCCGCT-3′ (contains a BbsI site and cleavage yields a NcoI compatible overhang) (SEQ ID NO:332) and 5′-ATAAGAATGCGGCCGCTTACTCTCCTTCAACCCGCA-3′ (contains a NotI site) (SEQ ID NO:333) are used to amplify the hom gene from plasmid MB4066. The resulting mutant population is digested with BbsI and NotI, ligated into NcoI/NotI digested episomal plasmid containing the trcRBS promoter in the MB4094 plasmid backbone, and transformed into C. glutamicum ATCC 13032. The transformed cells are plated on agar plates containing a defined medium for corynebacteria (see Guillouet, S., et al. Appl. Environ. Microbiol. 65:3100-3107, 1999) containing kanamycin (25 mg/L), 20 mg/L of AHV (alpha-amino, beta-hydroxyvaleric acid; a threonine analog) and 0.01 mM IPTG. After 72 h at 30° C., the resulting transformants are subsequently screened for homoserine excretion by replica plating to a defined medium agar plate supplemented with threonine, which was previously spread with ˜10⁶ cells of indicator C. glutamicum strain MA-331 (hom-thrBΔ). Putative feedback-resistant mutants are identified by a halo of growth of the indicator strain surrounding the replica-plated transformants. From each of these colonies, the hom gene is PCR amplified using the above primer pair, the amplicon is digested as above, and ligated into the episomal plasmid described above. Each of these putative hom mutants is subsequently re-transformed into C. glutamicum ATCC 13032 and plated on minimal medium agar plates containing 25 mg/L kanamycin and 0.01 mM IPTG. One colony from each transformation is replica plated to defined medium for corynebacteria containing 10, 20, 50, and 100 mg/L of AHV, and sorted based on the highest level of resistance to the threonine analog. Representatives from each group are grown in minimal medium to an OD of 2.0, the cells harvested by centrifugation, and homoserine dehydrogenase activity assayed in the presence and absence of 20 mM threonine as referenced in Chassagnole, C., et al., Biochem. J. 356:415-423, 2001. The hom gene is PCR amplified from those cultures showing feedback-resistance and sequenced. The resulting plasmids are used to generate expression plasmids to enhance amino acid production.

### Example 5

### Isolation of Feedback-Resistant Mutants of Homoserine O-Acetyltransferase (metA) and O-Acetylhomoserine Sulfhydrylase (metY)

The heterologous metA gene cloned from T. fusca is subjected to error prone PCR using the GeneMorph® Random Mutagenesis kit obtained from Stratagene. Under the conditions specified in this kit, oligonucleotide primers 5′-CACACACCTGCCACACATGAGTCACGACACCACCCCTCC-3′ (contains a BspMI site and cleavage yields a NcoI compatible overhang) (SEQ ID NO:334) and 5′-ATAAGAATGCGGCCGCTTACTGCGCCAGCAGTTCTT-3′ (contains a NotI site) (SEQ ID NO:335) are used to amplify the metA gene from plasmid MB4062. The resulting mutant amplicon is digested and ligated into the NcoI/NotI digested episomal plasmid described in Example 4, and then transformed into C. glutamicum strain MA-428. MA-428 is a derivative of ATCC 13032 that has been transformed with integrating plasmid MB4192. After selection for recombination events, the resulting strain MA-428 is deleted for hom-thrB in a manner that results in insertion of a deregulated S. coelicolor hom gene. The transformed MA-428 cells described are plated on minimal medium agar plates containing kanamycin (25 mg/L), 0.01 mM IPTG, and 100 μg/ml or 500 μg/ml of trifluoromethionine (TFM; a methionine analog). After 72 h at 30° C., the resulting transformants are subsequently screened for O-acetylhomoserine excretion by replica plating to a minimal agar plate which was previously spread with ˜10⁶ cells of an indicator strain, S. cerevisiae B-7588 (MATa ura3-52, ura3-58, leu2-3, leu2-112, trpl-289, met2, HIS3+), obtained from ATCC (#204524). Putative feedback-resistant mutants are identified by the excretion of O-acetylhomoserine (OAH), which supports a halo of indicator strain growth surrounding the replica-plated transformants.

From each of these cross-feeding colonies, the metA gene is PCR amplified using the above primer pair, digested with BspMI and NotI, and ligated into the NotI/NcoI digested episomal plasmid described in Example 4. Each of these putative metA mutant alleles is subsequently re-transformed into C. glutamicum ATCC 13032 and plated on minimal medium agar plates containing 25 mg/L kanamycin. One colony from each transformation is replica plated to minimal medium containing 100, 200, 500, and 1000 μg/ml of TFM plus 0.01 mM IPTG, and sorted based on the highest level of resistance to the methionine analog. Representatives from each group are grown in minimal medium to an OD of 2.0, the cells harvested by centrifugation, and homoserine O-acetyltransferase activity is determined by the methods described by Kredich and Tomkins (J. Biol. Chem. 241:4955-4965,1966) in the presence and absence of 20 mM methionine or S-AM. The metA gene is PCR amplified from those cultures showing feedback-resistance and sequenced. The resulting plasmids are used to generate expression plasmids to enhance amino acid production.

In a similar manner, the metY gene from T. fusca is subjected to mutagenic PCR. Oligonucleotide primers 5′-CACAGGTCTCCCATGGCACTGCGTCCTGACAGGAG-3′ (contains a BsaI site and cleavage yields a NcoI compatible overhang) (SEQ ID NO:336) and 5′-ATAAGAATGCGGCCGCTCACTGGTATGCCTTGGCTG-3′ (contains a NotI site) (SEQ ID NO:337) are used for cloning into the episomal plasmid, as described above, and for carrying out the mutagenesis reaction per the specifications of the GeneMorph® Random Mutagenesis kit obtained from Stratagene. The major difference is that the mutated metY population is transformed into a C. glutamicum strain that already produces high levels of O-acetylhomoserine. This strain, MICmet2, is constructed by transforming MA-428 with a modified version of plasmid MB4286 that contains a deregulated T. fusca metA allele described above under the control of the trcRBS promoter. After transformation the sacB selection system enables the deletion of the endogenous mcbR locus and replacement with the deregulated heterologous metA allele.

The T. fusca metY variant transformed MICmet2 strain is spread onto minimal agar plates containing 25 mg/L of kanamycin, 0.25mM IPTG, and an inhibiting concentration of toxic methionine analog(s) (e.g., ethionine, selenomethionine, TFM); the transformants can be grown on these 3 different methionine analogs either individually or in double or triple combination). The metY gene is amplified from those colonies growing on the selection plates, the amplicons are digested and ligated into the episomal plasmid described in Example 4, and the resulting plasmids are transformed into MICmet2. The transformants are grown on minimal medium agar plates containing 25 mg/L of kanamycin. The resulting colonies are replica-plated to agar plates containing a 10-fold range of the toxic methionine analogs ethionine, TFM, and selenomethionine (plus 0.01 mM IPTG), and sorted on the basis of analog sensitivity. Representatives from each group are grown in minimal medium to an OD of 2.0, the cells are harvested by centrifugation, and O-acetylhomoserine sulfhydrylase enzyme activity is determined by a modified version of the methods of Kredich and Tomkins (J. Biol. Chem. 241:4955-4965,1966) (see example 9) in the presence and absence of 20 mM methionine. The metY gene is PCR amplified from those cultures showing feedback-resistance and sequenced. The resulting plasmids are used to generate expression plasmids to enhance amino acid production. An expression plasmid containing the feedback resistant metY and metA variants from T. fusca is constructed as follows. The T. fusca metYA operon is amplified using oligonucleotides 5′-CACACACATGTCACTGCGTCCTGACAGGAGC-3′ (contains a Pcil site and cleavage yields a NcoI compatible overhang (also changes second codon from Ala>Ser)) (SEQ ID NO:338) and 5′-ATAAGAATGCGGCCGCTTACTGCGCCAGCAGTTCTT-3′ (contains a NotI site) (SEQ ID NO:339). The amplicon is digested with PciI and NotI, and the fragment is ligated into the above episomal plasmid that has been treated sequentially treated with NotI, HaeIII methylase, and NcoI. Site directed mutagenesis, performed using the QuikChange Site-Directed Mutagenesis Kit from Stratagene, is used to incorporate the described substitution mutations in T. fusca metA and metY into a single plasmid that expresses the deregulated alleles as an operon. The resulting plasmid is used to enhance amino acid production.

Minimal medium: 10 g glucose, 1 g NH₄H₂PO₄, 0.2 g KCl, 0.2 g MgSO₄-7H₂O, 30 μg biotin, and 1 ml TE per liter of deionized water (pH 7.2). Trace elements solution (TE) comprises: 88 mg Na₂B₄O₇-10H₂O, 37 mg (NH₄)₆Mo₇O₂₇-4H₂O, 8.8 mg ZnSO₄-7H₂O, 270 mg CuSO₄-5H₂O, 7.2 mg MnCl₂-4H₂O, and 970 mg FeCl₃-6H₂O per liter of deionized water. (When needed to support auxotrophic requirements, amino acids and purines are supplemented to 30 mg/L final concentration.)

### Example 6

### Identification of S-AM-Binding Residues in Bacterial Amino Acid Sequences

Many enzymes that regulate amino acid production are subject to allosteric feedback inhibition by S-AM. We hypothesized that variants of these enzymes with resistance to S-AM regulation (e.g., via resistance to S-AM binding or to S-AM-induced allosteric effects) would be resistant to feedback inhibition. S-AM binding motifs have been identified in bacterial DNA methyltransferases (Roth et al., J. Biol. Chem., 273:17333-17342, 1998). Roth et al. identified a highly conserved amino acid motif in EcoRV α-adenine-N⁶-DNA methyltransferase which appeared to be critical for S-AM binding by the enzyme. We searched for related motifs in the amino acid sequences of the following proteins of C. glutamicum: MetA, MetY, McbR, LysC, MetB, MetC, MetE, MetH, and MetK. Putative S-AM binding motifs were identified in MetA, MetY, McbR, LysC, MetB, MetC, MetH, and MetK. We also identified additional residues in metY that are analogous to a S-AM binding motif in a yeast protein. (Pintard et al., Mol. Cell Biol., 20(4): 1370-1381, 2000). Residues of each protein that may be involved in S-AM binding are listed in Table 16.

TABLE 16
Putative residues involved in S-AM binding in C. glutamicum proteins

| | Putative residue involved |
|---|---|
| Protein | in S-AM binding |
| MetA | G231 |
| | K233 |
| | F251 |
| | V253 |
| | D269 |
| MetY | G227 |
| | L229 |
| | D231 |
| | G232 |
| | G233 |
| | F235 |
| | D236 |
| | V239 |
| | F368 |
| | D370 |
| | D383 |
| | G346 |
| | K348 |
| McbR | G92 |
| | K94 |
| | F116 |
| | G118 |
| | D134 |
| LysC | G208 |
| | K210 |
| | F223 |
| | V225 |
| | D236 |
| MetB | G72 |
| | K74 |
| | F90 |
| | I92 |
| | D105 |
| MetC | G296 |
| | K298 |
| | F312 |
| | G314 |
| | D335 |
| MetH | G708 |
| | K710 |
| | F725 |
| | L727 |
| MetK | G263 |
| | K265 |
| | F282 |
| | G284 |
| | D291 |

Alignment of MetA and MetY sequences from other species was used to identify additional putative S-AM-binding residues. These residues are listed in Table 17.

TABLE 17
Putative S-AM binding amino acids in bacterial MetA and MetY proteins

| | | Putative residue | |
|---|---|---|---|
| | | involved in S-AM | Homologous Residue in |
| Protein | Organism | binding | C. glutamicum |
| MetY | T. fusca | G240 | G227 |
| | | D244 | D231 |
| | | F379 | F368 |
| | | D394 | D383 |
| MetY | M. tuberculosis | G231 | G227 |
| | | D235 | D231 |
| | | F367 | F368 |
| | | D382 | D383 |
| MetA | T. fusca | G81 | analogous residue absent in |
| | | | C. glutamicum |
| | | D287 | D269 |
| | | F269 | F251 |
| MetA | E. coli | E252 | D269 |
| MetA | M. leprae | G73 | analogous residue absent in |
| | | | C. glutamicum |
| | | D278 | D269 |
| | | Y260 | D269 |
| MetA | M. tuberculosis | G73 | analogous residue absent in |
| | | | C. glutamicum |
| | | Y260 | F251 |
| | | D278 | D269 |

MetA and MetY genes were cloned from C. glutamicum and T. fusca as described in Example 2. Table 11 lists the plasmids and strains used for the expression of wild-type and mutated alleles of MetA and MetY genes. Tables 18 and 19 list the plasmids used for expression and the oligonucleotides employed for site-directed mutagenesis to generate MetA and MetY variants.

### Example 7

### Preparation of Protein Extracts for MetA and MetY Assays

A single C. glutamicum colony was inoculated into seed culture media (see example 10 below) and grown for 24 hour with agitation at 33° C. The seed culture was diluted 1:20 in production soy media (40 mL) (example 10) and grown 8 hours. Following harvest by centrifugation, the pellet was washed 1× in 1 volume of water. The pellet was resuspended in 250 μl lysis buffer (1 ml HEPES buffer, pH 7.5, 0.5 ml 1M KOH, 10 μl 0.5M EDTA, water to 5 ml), 30 μl protease inhibitor cocktail, and 1 volume of 0.1 mm acid washed glass beads. The mixture was alternately vortexed and held on ice for 15 seconds each for 8 reptitions. After centrifugation for 5′ at 4,000 rpm, the supernatant was removed and re-spun for 20′ at 10,000 rpm. The Bradford assay was used to determine protein concentration in the cleared supernatant.

### Example 8

### Quantifying MetA Activity in C. glutamicum Strains Containing Episomal Plasmids

MetA activity in C. glutamicum expressing endogenous and episomal metA genes was determined. MetA activity was assayed in crude protein extracts using a protocol described by Kredich and Tomkins (J. Biol. Chem.241(21): 4955-4965, 1966). Preparation of protein extracts is described in the Example 7. Briefly, 1 μg of protein extract was added to a microtiter plate. Reaction mix (250 μl; 100 mM tris-HCl pH 7.5, 2 mM 5,5′-Dithiobis(2-nitrobenzoic acid) (DTN), 2 mM sodium EDTA, 2 mM acetyl CoA, 2 mM homoserine) was added to each well of the microtiter plate. In the course of the reactions, MetA activity liberates CoA from acetyl-CoA. A disulfide interchange occurs between the CoA and DTN to produce thionitrobenzoic acid. The production of thionitrobenzoic acid is followed spectrophotometrically. Absorbance at 412 nm was measured every 5 minutes over a period of 30 minutes. A well without protein extract was included as a control. Inhibition of MetA activity was determined by addition of S-adenosyl methionine (S-AM; 0.02 mM, 0.2 mM, 2 mM) and methionine (0.5 mM, 5 mM, 50 mM). Inhibitors were added directly to the reaction mix before it was added to the protein extract.

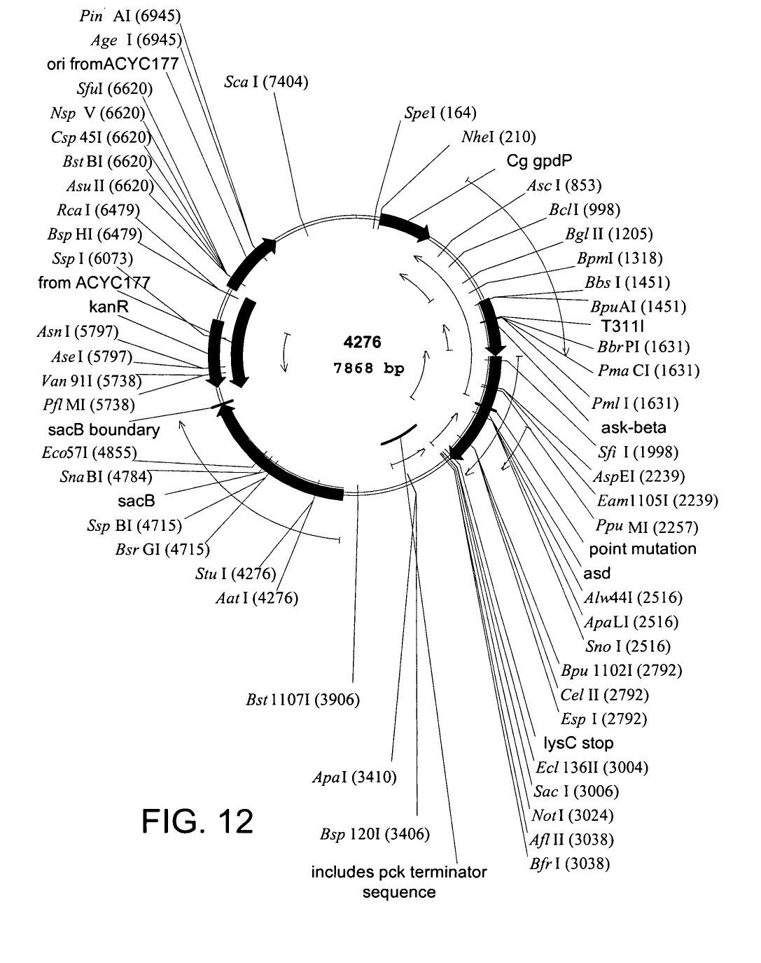

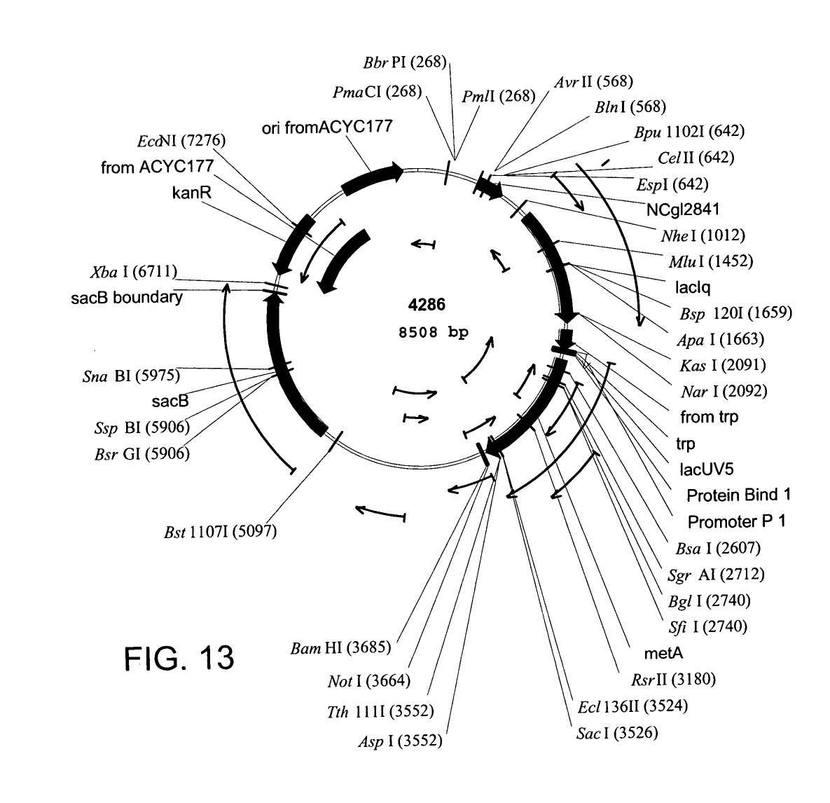

In vitro O-acetyltransferase activity was measured in crude protein extracts derived from C. glutamicum strains MA-442 and MA-449 which contain both endogenous and episomal C. glutamicum MetA and MetY genes. Episomal metA and metY genes were expressed as a synthetic operon; the nucleic acid sequence of the metAY operon is as shown in the metAYH operon of FIG. 12B, only lacking metH sequence. The trcRBS promoter was employed in these episomal plasmids. MA-442 expresses the episomal genes in the order metA-metY. MA-449 expresses the episomal genes in the order metY-metA. Experiments were performed in the presence and absence of IPTG that induces expression of the plasmid borne MetA and MetY genes. FIG. 13 shows a time course of MetA activity. MetA activity was observed only when the genes were in the MetA-MetY (MA-442) configuration in samples from 8 hour and 20 hour cultures. In contrast, MetA activity in extracts from strain MA-449 (MetY-MetA) was not significantly elevated relative to a control sample lacking protein at both 8 hour and 20 hour time points, with and without induction. This data is consistent with Northern blot analysis that showed low expression of metA when the two genes were in the metY-metA orientation.

Next, sensitivity of extracts from strain MA-442 to feedback inhibition was tested. MA-442 extracts were assayed in the presence of 5 mM methionine, 0.2 mM S-AM, or in the absence of additional methionine or S-AM, and MetA activity was assayed as described above. As shown in FIG. 14, MetA activity was reduced in the presence of 5 mM methionine and 0.2 mM S-AM. Thus, reducing allosteric repression of MetA may enhance MetA activity, allowing production of higher levels of methionine. It is possible that allosteric repression would also be observed at much lower levels of methionine or S-AM. Regardless, the levels tested are physiologically relevant levels in strains engineered for the production of amino acids such as methionine. C. glutamicum strains expressing episomal T. fusca MetA (strains MA-578 and MA-579), or both episomal T. fusca MetA and MetY (strains MA-456 and MA-570) were constructed and extracts were prepared from these strains and assayed for MetA activity. The regulatory elements associated with each episomal gene are listed in Table 18. The rate of MetA activity in extracts of each strain was determined by calculating the change in OD₄₁₂ divided by time per ng of protein. The results of these assays are depicted in FIG. 17, which shows that strain MA-578 exhibited a rate of approximately 2.75 units (change in OD₄₁₂/time/ng protein) under inducing conditions, whereas the rate under non-inducing conditions was approximately 1. Strain MA-579 exhibited a rate of approximately 2.5 under inducing conditions and a rate of approximately 0.4 under non-inducing conditions. Strain MA-456, which expresses metA and metY under the control of a constitutive promoter, exhibited a rate of approximately 2.2. Strain MA-570 exhibited a rate of approximately 1 under inducing conditions and a rate of 0.3 under non-inducing conditions. The negative control sample (no protein) exhibited a rate of approximately 0.1. These data show that episomal expression of T. fusca metA in C. glutamicum increases the rate of MetA activity. The increase was similar to the increase observed with episomal expression of C. glutamicum MetA in C. glutamicum.

### Example 9

### Quantifying MetY Activity in C. glutamicum Strains Containing Episomal Plasmids

The in vitro activity of episomal T. fusca MetY was determined in several C. glutamicum strains. MetY activity was assayed in C. glutamicum crude protein extracts using a modified protocol of Kredich and Tomkins (J. Biol. Chem., 241(21): 4955-4965, 1966). Crude protein extracts were prepared as described. Briefly, 900 μl of reaction mix (50 mM Tris pH 7.5, 1 mM EDTA, 1 mM sodium sulfide nonahydrate (Na₂S), 0.2 mM pyridoxal-5-phosphoric acid (PLP) was mixed with 45 μg of protein extract. At time zero, O-acetyl homoserine (OAH; Toronto Research Chemicals Inc) was added to a final concentration of 0.625 mM. 200 μl of the reaction was removed immediately for the zero time point. The remainder of the reaction was incubated at 30° C. Three 200 μl samples were removed at 10 minute intervals. Immediately after removal from 30° C., the reactions were stopped by the addition of 125 μl 1 mM nitrous acid which nitrosates the thiol groups of homocysteine to form S-nitrosothiol. Five minutes later, 30 μl of 0.5% ammonium sulfamate (removes excess nitrous acid) was added and the sample vortexed. Two minutes later, 400 μl of detection solution (1 part 1% HgCl2 in 0.4N HCl, 4 parts 3.44% % sulfanilamide in 0.4N HCl, 2 parts 0.1% 1-naphthylethylenediamine dihydrochloride in 0.4N HCl) was added and the solution vortexed. In the presence of mercuric ion the S-nitrosothiol rapidly decomposes to give nitrous acid, diazotizing the sulfanilamide, which then couples with the naphthylethylenediamine to give a stable azo dye as a chromaphore. After 5 minutes, the solution was transferred to a microtiter dish and the absorbance at 540 nm was measured. A reaction without protein extract was included as a control.

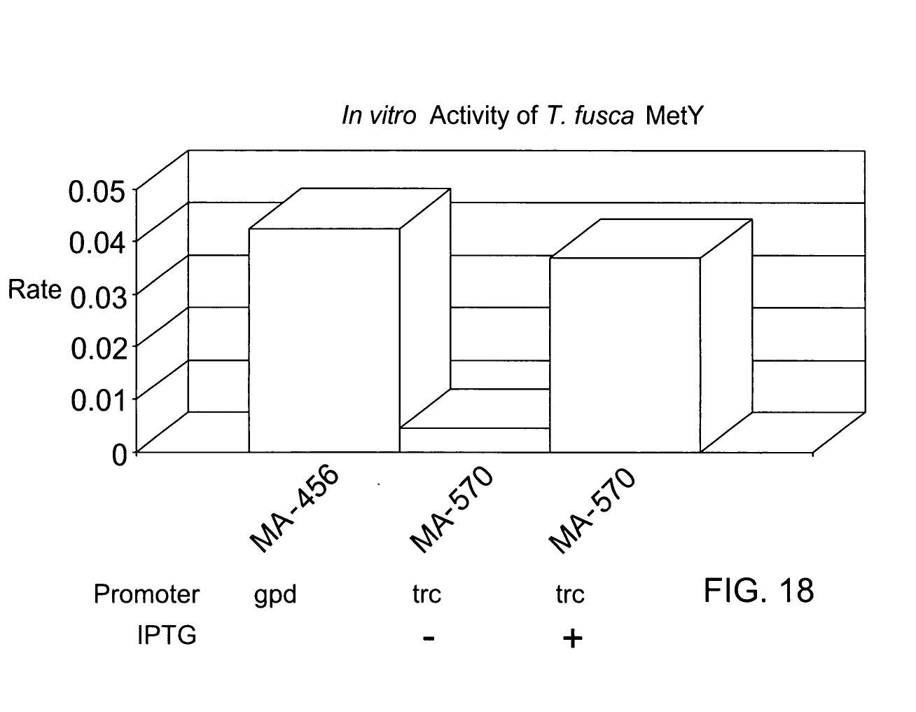

The results of the assays are depicted in FIG. 18. Strain MA-456, which expresses episomal wild type T. fusca metA and metY alleles under the control of a constitutive promoter, exhibited a rate of 0.04. Strain MA-570, which expresses episomal wild type T. fusca metA and metY alleles under the control of an inducible promoter, exhibited a rate of approximately 0.038 under inducing conditions, and a rate of less than 0.01 under non-inducing conditions. Thus, expression of heterologous MetY results in enzyme activity that is significantly elevated over that of the endogenous MetY.

TABLE 18
C. glutamicum strains used to determine activity of MetA and MetY
proteins, and impact of overexpression on production of aspartate-derived amino acids.

| | relevant | | relevant plasmid | episomal | |
|---|---|---|---|---|---|
| Strain | strain | episomal | regulatory | metY | episomal |
| Name | genotype | plasmid | sequence | species | metA species |
| MA-2 | n/a | n/a | n/a | n/a | n/a |
| (ATCC | | | | | |
| 13032) | | | | | |
| MA-422 | ethionine | n/a | n/a | n/a | n/a |
| | resistant | | | | |
| | variant of | | | | |
| | MA-2 | | | | |
| MA-428 | MA-2 | n/a | n/a | n/a | n/a |
| | derivative | | | | |
| | with Δhom- | | | | |
| | ΔthrB::C glutamicum | | | | |
| | gpd promoter- | | | | |
| | S. coelicolor | | | | |
| | hom | | | | |
| | (G362E)^{a} | | | | |
| MA-442 | MA-428 | MB-4135^{b} | lacIQ-TrcRBS | Cg wild- | Cg wild-type |
| | derivative | | | type | |
| MA-449 | MA-428 | MB-4138 | lacIQ-TrcRBS | Cg wild- | Cg wild-type |
| | derivative | | | type | |
| MA-456 | MA-428 | MB-4168 | gpd | Tf wild-type | Tf wild-type |
| | derivative | | | | |
| MA-570 | MA-428 | MB-4199 | lacIQ-TrcRBS | Tf wild-type | Tf wild-type |
| | derivative | | | | |
| MA-578 | MA-428 | MB-4205 | gpd | none | Tf wild-type |
| | derivative | | | | |
| MA-579 | MA-428 | MB-4207 | lacIQ-TrcRBS | none | Tf wild-type |
| | derivative | | | | |
| MA-622 | mcbRΔ | n/a | n/a | n/a | n/a |
| | derivative of | | | | |
| | MA-422 | | | | |
| MA-641 | MA-622 | MB-4136 | gpd | Cg wild- | Cg wild-type |
| | derivative | | | type | |
| MA-699 | MA-622 | n/a | n/a | n/a | n/a |
| | derivative | | | | |
| MA-721 | MA-622 | MB-4236^{b} | lacIQ-TrcRBS | Cg wild- | Cg K233A |
| | derivative | | | type | |
| MA-725 | MA-622 | MB-4238^{b} | lacIQ-TrcRBS | Cg D231A | Cg wild-type |
| | derivative | | | | |
| MA-727 | MA-622 | MB-4239^{b} | lacIQ-TrcRBS | Cg G232A | Cg wild-type |
| | derivative | | | | |

abbreviations - Cg (Coryneform glutamicum), Tf (Thermobifida fusca), lacIQ-TrcRBS (see above) (lacIQ-Trc regulatory sequence from pTrc99A (Amann et al., Gene (1988) 69: 301-315)); gpd (C. glutamicum gpd promoter)
^{a}the endogenous hom(thrA)-thrB locus was replaced with the S. coelicolor hom (G362E) sequence under the C. glutamicum gpd (glyceraldehyde-3-phosphate dehydrogenase) promoter
^{b}in this plasmid the gene order is MetA-MetY. Unless otherwise indicated, in other plasmids the gene order is MetY-MetA

TABLE 19
Plasmids and oligos used for site directed mutagenesis to generate
MetA and MetY variants.

| Plasmid | oligo 1 | oligo 2 | Gene | wt/variant | Organism |
|---|---|---|---|---|---|
| MB4238 | MO4057 | MO4058 | metY | D231A | C. glutamicum |
| n/a | MO4045 | MO4046 | metY | D244A | T. fusca |
| n/a | MO4041 | MO4042 | metA | D287A | T. fusca |
| n/a | MO4049 | MO4050 | metY | D394A | T. fusca |
| n/a | MO4039 | MO4040 | metA | F269A | T. fusca |
| n/a | MO4047 | MO4048 | metY | F379A | T. fusca |
| MB4239 | MO4059 | MO4060 | metY | G232A | C. glutamicum |
| n/a | MO4043 | MO4044 | metY | G240A | T. fusca |
| n/a | MO4037 | MO4038 | metA | G81A | T. fusca |
| MB4236 | MO4051 | MO4052 | metA | K233A | C. glutamicum |
| MB4135 | n/a | n/a | metA | wt | C. glutamicum |
| MB4135 | n/a | n/a | metY | wt | C. glutamicum |
| MB4210 | n/a | n/a | metY | wt | T. fusca |
| MB4210 | n/a | n/a | metA | wt | T. fusca |

TABLE 20

Sequences of oligos used for site-directed mutagenesis to
generate MetA and MetY variants.

| | | |
|---|---|---|
| Oligo name | Oligo Sequence | SEQ ID NO: |

| | | |
|---|---|---|
| M04037 | 5′ GTAGGCCCGGAAGGCCCCGCGCACCCCAGCCCAGGCTGG 3′ | 340 |
| M04038 | 5′ CCAGCCTGGGCTGGGGTGCGCGGGGCCTTCCGGGCCTAC 3′ | 341 |
| M04039 | 5′ CCGATGGCCGGGGGCCGGGCCGCTGTCGAGTCGTACCTG 3′ | 342 |
| M04040 | 5′ CAGGTACGACTCGACAGCGGCCCGGCCCCCGGCCATCGG 3′ | 343 |
| M04041 | 5′ AAACTCGCCCGCCGGTTCGCCGCGGGCAGCTACGTCGTG 3′ | 344 |
| M04042 | 5′ CACGACGTAGCTGCCCGCGGCGAACCGGCGGGCGAGTTT 3′ | 345 |
| M04043 | 5′ CACGGCACCACGATCGCGGCCATCGTGGTGGACGCCGGC 3′ | 346 |
| M04044 | 5′ GCCGGCGTCCACCACGATGGCCGCGATCGTGGTGCCGTG 3′ | 347 |
| M04045 | 5′ ATCGCGGGCATCGTGGTGGCCGCCGGCACCTTCGACTTC 3′ | 348 |
| M04046 | 5′ GAAGTCGAAGGTGCCGGCGGCCACCACGATGCCCGCGAT 3′ | 349 |
| M04047 | 5′ ATCGAGGCCGGACGCGCCGCCGTGGACGGCACCGAACTG 3′ | 350 |
| M04048 | 5′ CAGTTCGGTGCCGTCCACGGCGGCGCGTCCGGCCTCGAT 3′ | 351 |
| M04049 | 5′ CAGCTCGTCAACATCGGTGCCGTGCGCAGCCTCATCGTC 3′ | 352 |
| M04050 | 5′ GACGATGAGGCTGCGCACGGCACCGATGTTGACGAGCTG 3′ | 353 |
| M04051 | 5′ GACGAACGCTTCGGCACCGCAGCCCAAAAGAACGAAAAC 3′ | 354 |
| M04052 | 5′ GTTTTCGTTCTTTTGGGCTGCGGTGCCGAAGCGTTCGTC 3′ | 355 |
| M04057 | 5′ CTGGGCGGCGTGCTTATCGCCGGCGGAAAGTTCGATTGG 3′ | 356 |
| M04058 | 5′ CCAATCGAACTTTCCGCCGGCGATAAGCACGCCGCCCAG 3′ | 357 |
| M04059 | 5′ GGCGGCGTGCTTATCGACGCCGGAAAGTTCGATTGGACT 3′ | 358 |
| M04060 | 5′ AGTCCAATCGAACTTTCCGGCGTCGATAAGCACGCCGCC 3′ | 359 |

### Example 10

### Methods for Producing and Detecting Aspartate-Derived Amino Acids

For shake flask production of aspartate-derived amino acids, each strain was inoculated from an agar plate into 10 ml of Seed Culture Medium in a 125 ml Erlenmeyer flask. The seed culture was incubated at 250 rpm on a shaker for 16 h at 31° C. A culture for monitoring amino acid production was prepared by performing a 1:20 dilution of the seed culture into 10 ml of Batch Production Medium in 125 ml Erlenmeyer flasks. When appropriate, IPTG was added to a set of the cultures to induce expression of the IPTG regulated genes (final concentration 0.25 mM). Methionine fermentations were carried out for 60-66 h at 31° C. with agitation (250 rpm). For the studies reported herein, in nearly all instances, multiple transformants were fermented in parallel, and each transformant was often grown in duplicate. Most reported data points reflect the average of at least two fermentations with a representative transformant, together with control strains that were grown at the same time.

After cultivation, amino acid levels in the resulting broths were determined using liquid chromatography-mass spectrometry (LCMS). Approximately 1 ml of culture was harvested and centrifuged to pellet cells and particulate debris. A fraction of the resulting supernatant was diluted 1:5000 into aqueous 0.1 % formic acid and injected in 10 μL portions onto a reverse phase HPLC column (Waters Atlantis C 18, 2.1×150 mm). Compounds were eluted at a flow rate of 0.350 mL min⁻¹, using a gradient mixture of 0.1% formic acid in acetonitrile (“B”) and 0.1% formic acid in water (“A”), (1% B→50% B over 4 minutes, hold at 50% B for 0.2 minutes, 50% B→1% over 1 minute, hold at 1% for 1.8 minutes). Eluting compounds were detected with a triple-quadropole mass spectrometer using positive electrospray ionization. The instrument was operated in MRM mode to detect amino acids (lysine: 147→84 (15 eV); methionine: 150→104 (12 eV); threonine/homoserine: 120→74 (10 eV); aspartic acid: 134→88 (15 eV); glutamic acid: 148→84 (15 eV); O-acetylhomoserine: 162→102 (12 eV); and homocysteine: 136→90 (15 eV)). On occasion, additional amino acids were quantified using similar methods (e.g. homocystine, glycine, S-adenosylmethionine). Individual amino acids were quantified by comparison with amino acid standards injected under identical conditions. Using this mass spectrometric method it is not possible to distinguish between homoserine and threonine. Therefore, when necessary, samples were also derivatized with a fluorescent label and subjected to liquid chromatography followed by fluorescent detection. This method was used to both resolve homoserine and threonine as well as to confirm concentrations determined using the LCMS method.

Seed Culture Medium for Production Assays

| | | |
|---|---|---|
| Glucose | 100 | g/L |
| Ammonium acetate | 3 | g/L |
| KH₂PO₄ | 1 | g/L |
| MgSO₄-7H₂O | 0.4 | g/L |
| FeSO₄-7H₂O | 10 | mg/L |
| MnSO₄-4H₂O | 10 | mg/L |
| Biotin | 50 | μg/L |
| Thiamine-HCl | 200 | μg/L |
| Soy protein | 15 | ml/L |
| hydrolysate (total nitrogen 7%) | | |
| Yeast extract | 5 | g/L |
| pH 7.5 | | |

Batch Production Medium for Production Assays

| | | |
|---|---|---|
| Glucose | 50 | g/L |
| (NH₄)₂SO₄ | 45 | g/L |
| KH₂PO₄ | 1 | g/L |
| MgSO₄-7H₂O | 0.4 | g/L |
| FeSO₄-7H₂O | 10 | mg/L |
| MnSO₄-4H₂O | 10 | mg/L |
| Biotin | 50 | μg/L |
| Thiamine-HCl | 200 | μg/L |
| Soy protein | 15 | ml/L |
| hydrolysate (total nitrogen 7%) | | |
| CaCO₃ | 50 | g/L |
| Cobalamin | 1 | μg/ml |
| pH 7.5 | | |

(cobalamin addition not necessary when lysine is the target aspartate-derived amino acid)

### Example 11

### Heterologous Wild-Type and Mutant lysC Variants Increase Lysine Production in C. glutamicum and B. lactofermentum.

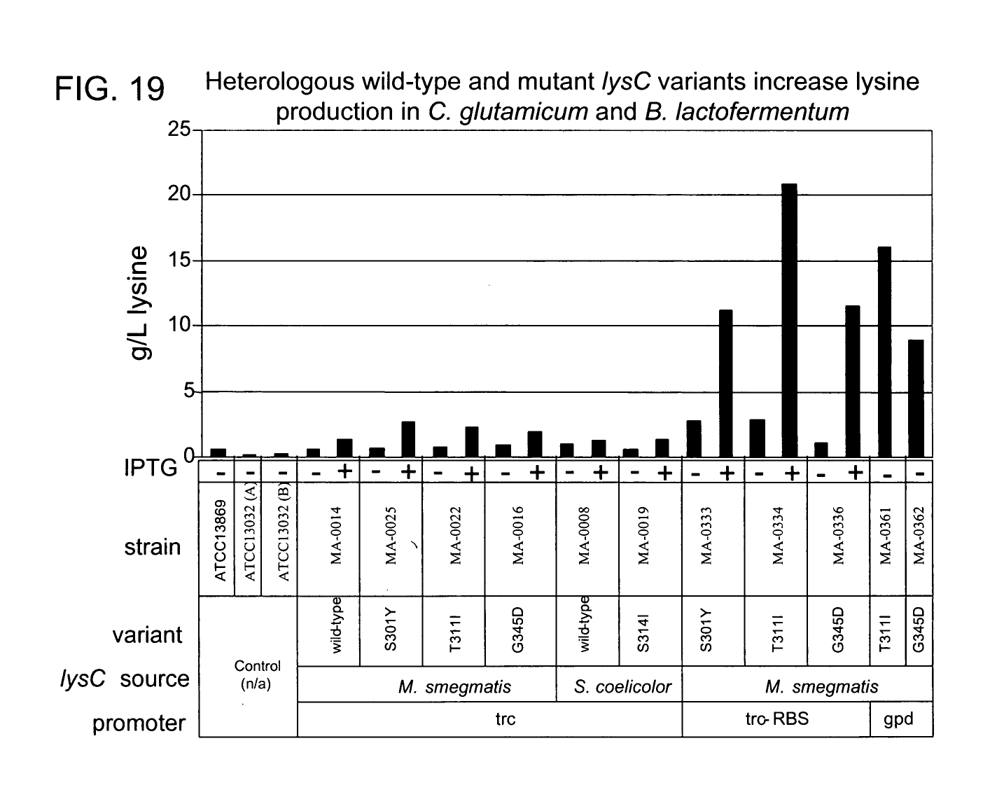

Aspartokinase is often the rate-limiting activity for lysine production in corynebacteria. The primary mechanism for regulating aspartokinase activity is allosteric regulation by the combination of lysine and threonine. Heterologous operons encoding aspartokinases and aspartate semi-aldehyde dehydrogenases were cloned from M. smegmatis and S. coelicolor as described in Example 2. Site-directed mutagenesis was used to generate deregulated alleles (see Example 3), and these modified genes were inserted into vectors suitable for expression in corynebacteria (Example 1). The resulting plasmids, and the wild-type counterparts, were transformed into strains, including wild-type C. glutamicum strain ATCC 13032 and wild-type B. lactofermentum strain ATCC 13869, which were analyzed for lysine production (FIG. 19).

Strains MA-0014, MA-0025, MA-0022, MA-0016, MA-0008 and MA-0019 contain plasmids with the MB3961 backbone (see Example 1). Increased expression, via addition of IPTG to the production medium, of either wild-type or deregulated heterologous lysC-asd operons promoted lysine production. Strain ATCC 13869 is the untransformed control for these strains. The plasmids containing M. smegmatis S301Y, T311I, and G345D alleles were most effective at enhancing lysine production; these alleles were chosen for expression for expression from improved vectors. Improved vectors containing deregulated M. smegmatis alleles were transformed into C. glutamicum (ATCC 13032) to generate strains MA-0333, MA-0334, MA-0336, MA-0361, and MA-0362 (plasmids contain either trcRBS or gpd promoter, MB4094 backbone; see Example 1). Strain ATCC 13032 (A) is the untransformed control for strains MA-0333, MA-0334 and MA-0336. Strain ATCC 13032 (B) is the untransformed control for strains MA-0361 and MA-0362.Strains MA-0333, MA-0334, MA-0336, MA-0361, and MA-0362 all displayed improvement in lysine production. For example, strain MA-0334 produced in excess of 20 g/L lysine from 50 g/L glucose. In addition, the T311I and G345D alleles were shown to be effective when expressed from either the trcRBS or gpd promoter.

### Example 12

### S. coelicolor Hom G362E Variant Increases Carbon Flow to Homoserine in C. glutamicum Strain MA-0331

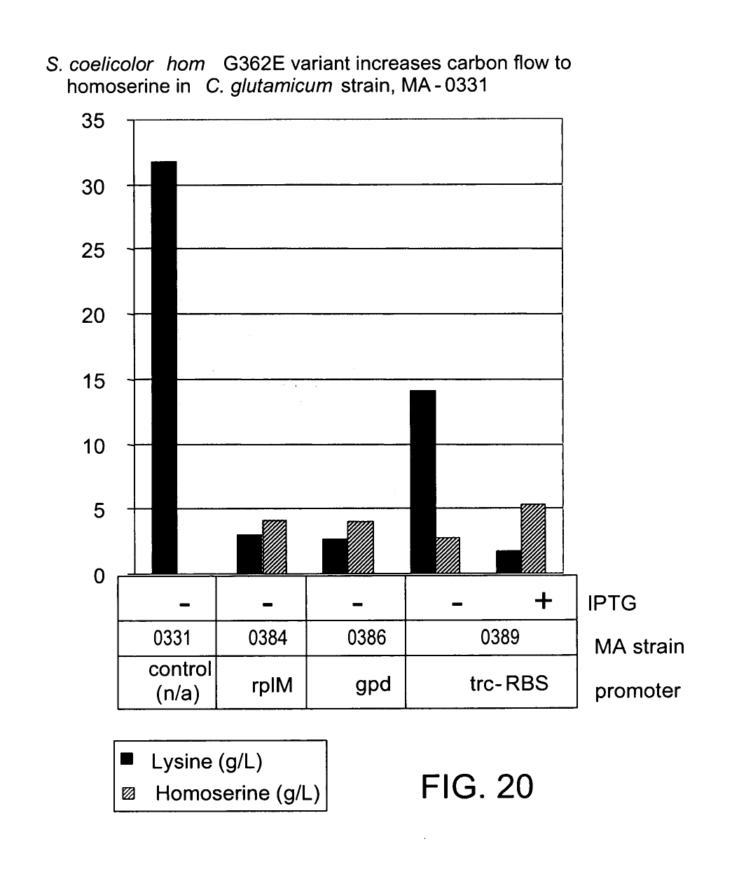

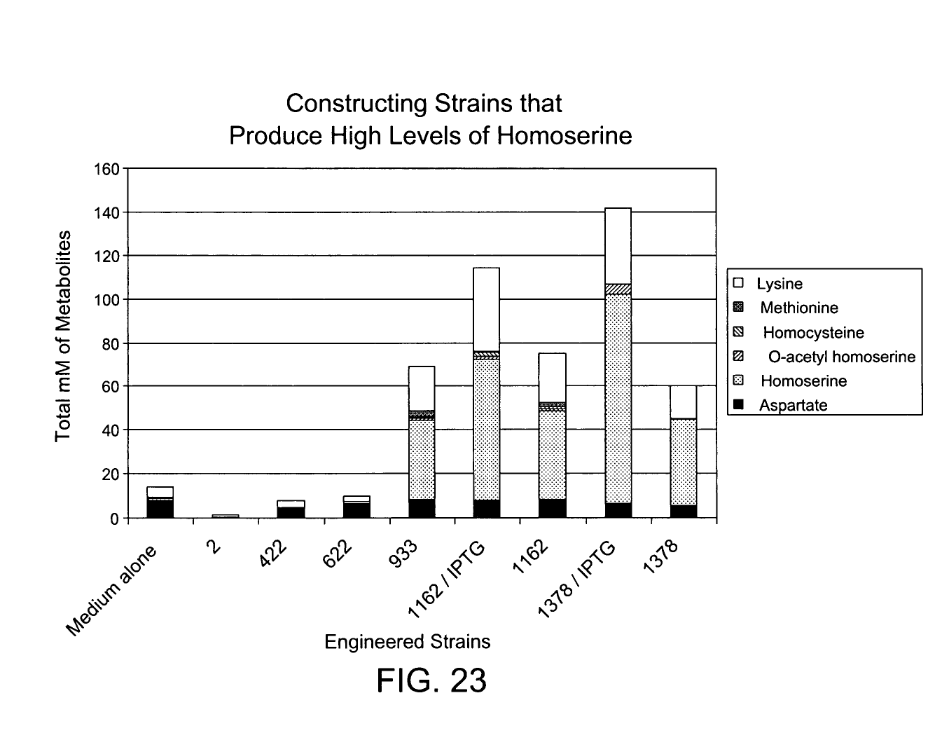

As shown in Example 11, deregulation of aspartokinase increased carbon flow to aspartate-derived amino acids. In principle, aspartokinase activity could be increased by the use of deregulated lysC alleles and/or by elimination of the small molecules that mediate the allosteric regulation (lysine or threonine). FIG. 20 (strain MA-0331) shows that high levels of lysine accumulated in the broth when the hom-thrB locus was inactivated. Hom and thrB encode for homoserine dehydrogenase and homoserine kinase, respectively, two proteins required for the production of threonine. Lysine accumulation was also observed when only the thrB gene was deleted (see strain MA-0933 in FIG. 23 (MA-0933 is one example, though it is not appropriate to directly compare MA-0933 to MA-0331, as these strains are from different genetic backgrounds).

In order to increase carbon flow to methionine pathway intermediates, a putative deregulated variant of the S. coelicolor hom gene was transformed into MA-0331. Similar strategies were used to engineer strains containing only the thrB deletion. Strains MA-0384, MA-0386, and MA-0389 contain the S. coelicolor homG362E variant under the control of the rplM, gpd, and trcRBS promoters, respectively. These plasmids also contain an additional substitution (G43S) that was introduced as part of the site-directed mutagenesis strategy; subsequent experiments suggested that the G43S substitution does not enhance Hom activity. FIG. 18 shows the results from shake flask experiments performed using strains MA-0331, MA-0384, MA-0386, and MA-0389, in whichbroths were analyzed for aspartate-derived amino acids, including lysine and homoserine. Strains expressing the S. coelicolor homG362E gene display a dramatic decrease in lysine production as well as a significant increase in homoserine levels. Broth levels of homoserine were in excess of 5 g/L in strains such as MA-0389. It is possible that significant levels of homoserine still remain within the cell or that some homoserine has been converted to additional products. Overexpression of deregulated lysC and other genes downstream of hom, together with hom, may increase production of homoserine-based amino acids, including methionine (see below).

### Example 13

### Heterologous Phosphoenolpyruvate Carboxylase (Ppc) Enzymes Increase Carbon Flow to Aspartate-Derived Amino Acids

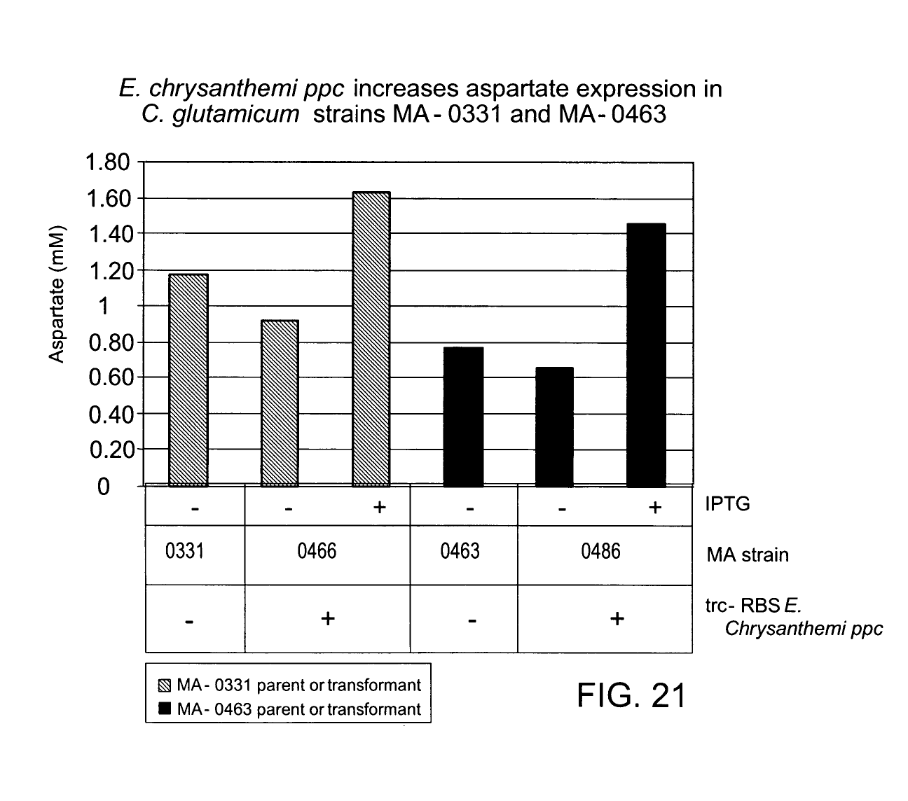

Phosphoenolpyruvate carboxylase (Ppc), together with pyruvate carboxylase (Pyc), catalyze the synthesis of oxaloacetic acid (OAA), the citric acid cycle intermediate that feeds directly into the production of aspartate-derived amino acids. The wild-type E. chrysanthemi ppc gene was cloned into expression vectors under control of the IPTG inducible trcRBS promoter. This plasmid was transformed into high lysine strains MA-0331 and MA-0463 (FIG. 21). Strains were grown in the absence or presence of IPTG and analyzed for production of aspartate-derived amino acids, including aspartate. Strain MA-0331 contains the hom-thrBA mutation, whereas MA-0463 contains the M. smegmatis lysC (T311I)-asd operon integrated at the deleted hom-thrB locus; the lysC-asd operon is under control of the C. glutamicum gpd promoter. FIG. 21 shows that the E. chrysanthemi ppc gene increased the accumulation of aspartate. This difference was even detectable in strains that converted most of the available aspartate into lysine.

### Example 14

### Heterologous Dihydrodipicolinate Synthases (dapA) Enzymes Increase Lysine Production.

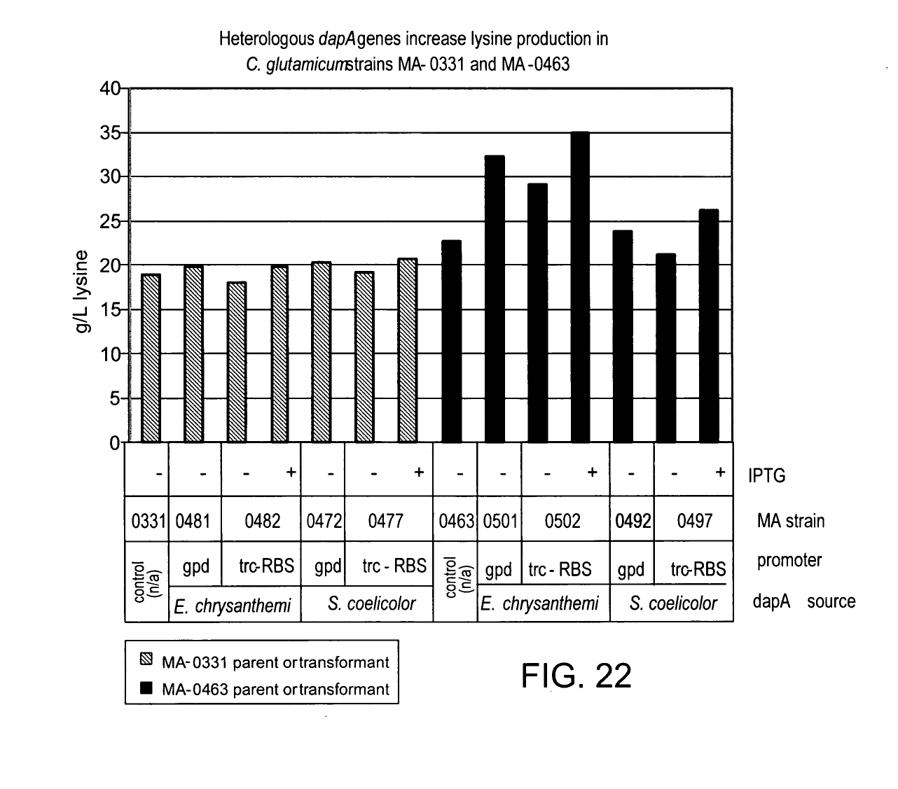

Dihydrodipicolinate synthase is the branch point enzyme that commits carbon to lysine biosynthesis rather than to the production of homoserine-based amino acids. DapA converts aspartate-B-semialdehyde to 2,3-dihydrodipicolinate. The wild-type E. chrysanthemi and S. coelicolor dapA genes were cloned into expression vectors under the control of the trcRBS and gpd promoters. The resulting plasmids were transformed into strains MA-0331 and MA-0463, two strains that had already been engineered to produce high levels of lysine (see Example 13). MA-0463 was engineered for increased expression of the M. smegmatis lysC(T311I)-asd operon. This manipulation is expected to drive production of aspartate-B-semialdehyde, the substrate for the DapA catalyzed reaction. Strains MA-0481, MA-0482, MA-0472, MA-0501, MA-0502, MA-0492, MA-0497 were grown in shake flask, and the broths were analyzed for aspartate-derived amino acids, including lysine. As shown in FIG. 22, increased expression of either the E. chrysanthemi or S. coelicolor dapA gene increases lysine production in the MA-0331 and MA-0463 backgrounds. Strain MA-0502 produced nearly 35 g/L lysine in a 50 g/L glucose process. It may be possible to engineer further lysine improvements by constructing deregulated variants of these heterologous dapA genes.

### Example 15

### Constructing Strains that Produce High Levels of Homoserine

Strains that produce high levels of homoserine-based amino acids can be generated through a combination of genetic engineering and mutagenesis strategies. As an example, five distinct genetic manipulations were performed to construct MA-1378, a strain that produces >10 g/L homoserine (FIG. 23). To generate MA-1378, wild-type C. glutamicum was first mutated using nitrosoguanidine (NTG) mutagenesis (based on the protocol described in “A short course in bacterial genetics.” J. H. Miller. Cold Spring Harbor Laboratory Press. 1992, page 143) followed by selection of colonies that grew on minimal plates containing high levels of ethionine, a toxic methionine analog. The endogenous mcbR locus was then deleted in one of the resulting ethionine-resistant strains (MA-0422) using plasmid MB4154 in order to generate strain MA-0622. McbR is a transcriptional repressor that regulates the expression of several genes required for the production of sulfur-containing amino acids such as methionine (see Rey, D.A., Puhler, A., and Kalinowski, J., J. Biotechnology 103:51-65, 2003). In several instances we observed that inactivation of McbR generated strains with increased levels of homoserine-based amino acids. Plasmid MB4084 was utilized to delete the thrB locus in MA-0622, causing the accumulation of lysine and homoserine; methionine and methionine pathway intermediates also accumulated to a lesser degree. MA-0933 resulted from this manipulation. As described above, it is believed that the lysine and homoserine accumulation was a result of deregulation of lysC, via the lack of threonine production. In order to further optimize carbon flow to aspartate-B-semialdehyde and downstream amino acids, MA-0933 was transformed with an episomal plasmid expressing the M. smegmatis lysC (T311I)-asd operon (strain MA-1162). High homoserine producing strain MA-1162 was then mutagenized with NTG, and colonies were selected on minimal medium plates containing a level of methionine methylsulfonium chloride (MMSC) that is normally inhibitory to growth. MA-1378 was one such MMSC-resistant strain.

### Example 16

### Heterologous Homoserine Acetyltransferases (MetA) Enzymes Increase Carbon Flow to Homoserine-Based Amino Acids

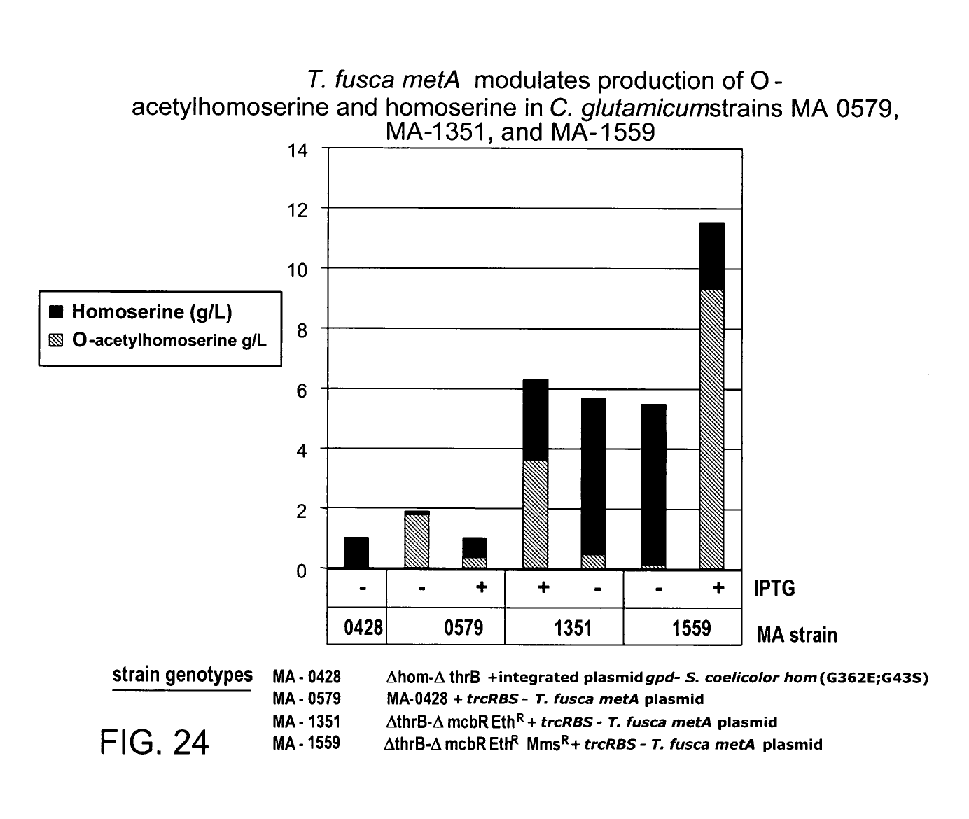

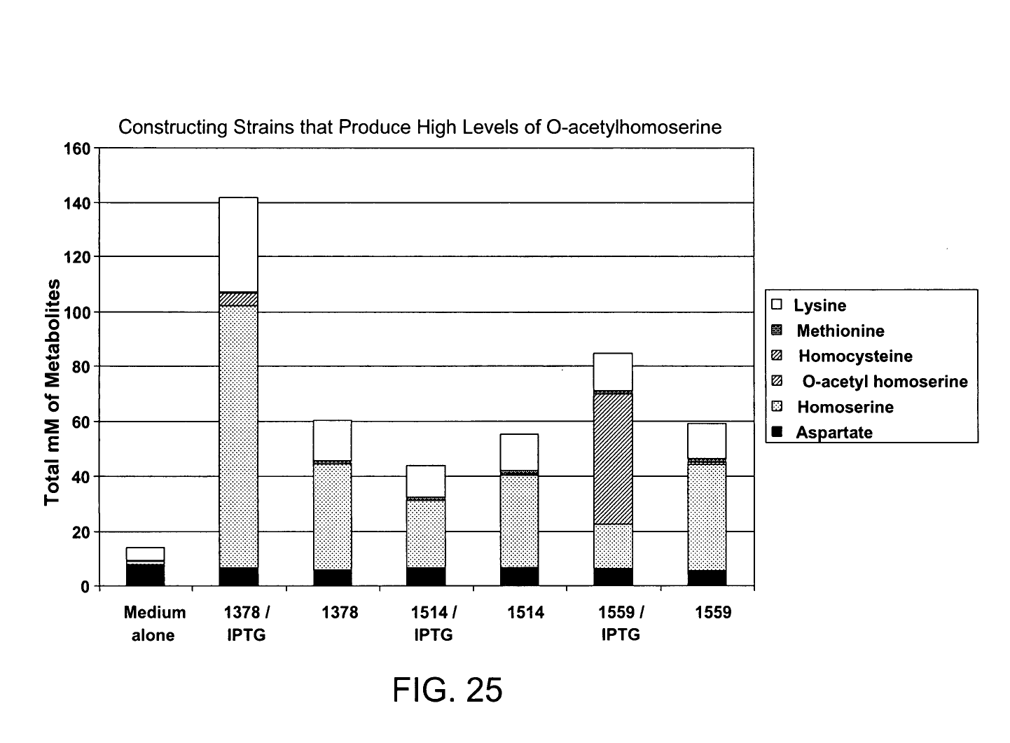

MetA is the commitment step to methionine biosynthesis. The wild-type T. fusca metA gene was cloned into an expression vector under the control of the trcRBS promoter. This plasmid was transformed into high homoserine producing strains to test for elevated MetA activity (FIGS. 24 and 25). MA-0428, MA-0933, and MA-1514 were example high homoserine producing strains. MA-0428 is a wild-type ATCC 13032 derivative that has been engineered with plasmid MB4192 (see Example 1) to delete the hom-thrB locus and integrate the gpd-S. coelicolor hom(G362E) expression cassette. MA-1514 was constructed by using novobiocin to allow for loss of the M. smegmatis lysC(T311 I)-asd operon plasmid from strain MA-1378. This manipulation was performed to allow for transformation with the episomal plasmid containing the T. fusca metA gene and the kanR selectable marker. Strain MA-1559 resulted from the transformation of strain MA-1514 with the T. fusca metA gene under control of the trcRBS promoter. MA-0933 is as described in Example 15. Induction of T. fusca metA expression in each of these high homoserine strains resulted in accumulation of O-acetylhomoserine in culture broths. For example, strain MA-1559 displayed OAH levels in excess of 9 g/L. Additional manipulations can be performed to elicit conversion of OAH to other products, including methionine.

### Example 17A

### Effects of MetA Variants on Methionine Production in C. glutamicum.

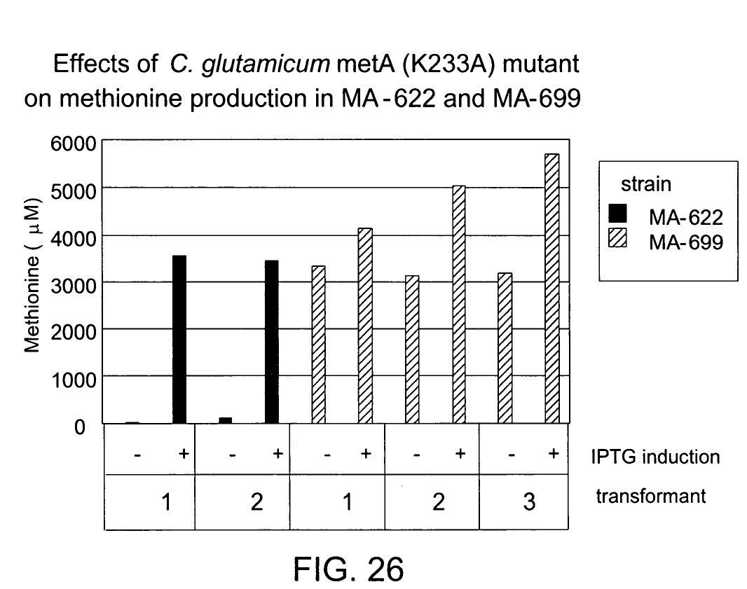

C. glutamicum homoserine acetyltransferase (MetA) variants were generated by site-directed mutagenesis of MetA-encoding DNA (Example 6). C. glutamicum strains MA-0622 and MA-0699 were transformed with a high copy plasmid, MB4236, that encodes MetA with a lysine to alanine mutation at position 233 (MetA (K233A)). This plasmid also contains a wild-type copy of the C. glutamicum metY gene. Strain MA-0699 was constructed by transforming MA-0622 with plasmid MB4192 to delete the hom-thrB locus and integrate the gpd-S. coelicolor hom(G362E) expression cassette. metA and metY are expressed in a synthetic metAY operon under control of a modified version of the trc promoter. The strains were cultured in the presence and absence of IPTG induction, and methionine productivity was assayed. Methionine production from each strain is plotted in FIG. 26. As shown, individual transformants of MA-622 and MA-699, when cultured under inducing conditions, each produced over 3000 μM methionine. MA-699 strains, which express an S. coelicolor hom G362E variant under the control of a constitutive promoter, produced over 3000 μM methionine in the absence of IPTG. IPTG induction resulted in an increased methionine production by 1000-2500 μM. These data show that expression of MetA (K233A) enhances methionine production. Manipulation of methionine biosynthesis at multiple points can further enhance production.

### Example 17B

### Effects of MetY Variants on Methionine Production in C. glutamicum

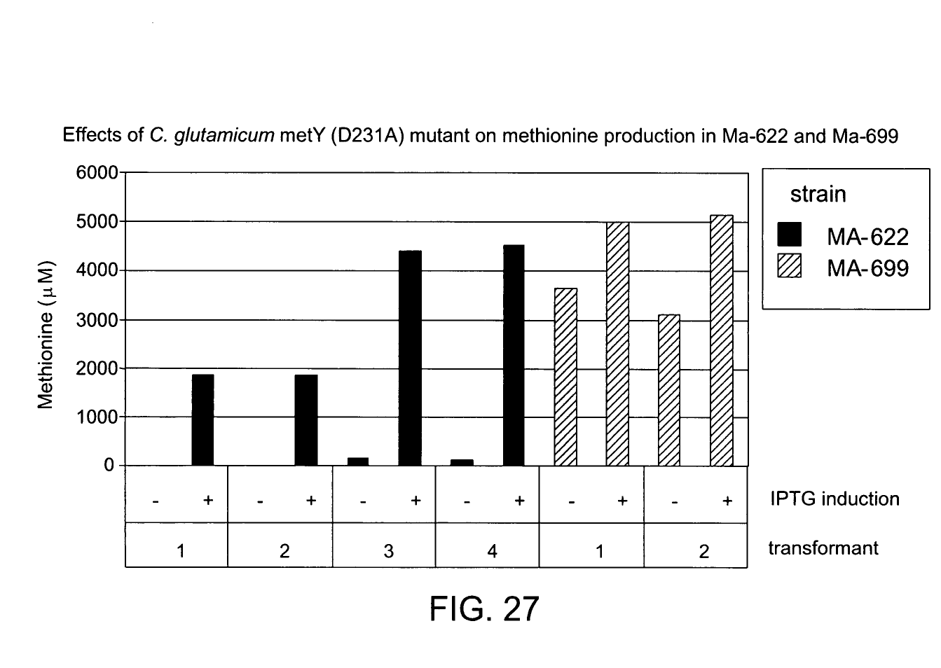

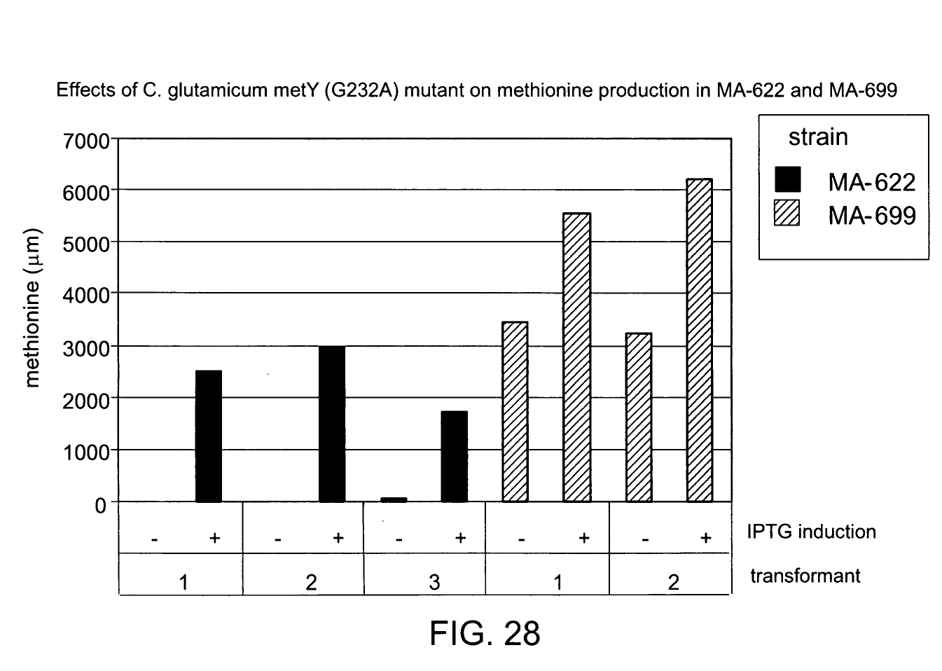

C. glutamicum O-acetylhomoserine sulfhydrylase (MetY) variants were generated by site-directed mutagenesis of MetY-encoding DNA (Example 6). C. glutamicum strain MA-622 and strain MA-699 were transformed with a high copy plasmid, MB4238, that encodes MetY with an aspartate to alanine mutation at position 231 (MetY (D231 A)). This plasmid also contains the wild-type copy of the C. glutamicum metA gene, expressed as in Example 16. The strains were cultured in the presence and absence of IPTG induction, and methionine productivity was assayed. The methionine production from each strain is plotted in FIG. 27. As shown, individual transformants of MA-622, when cultured under conditions in which expression of MetY (D231 A) was induced, each produced over 1800 μM methionine. MA-622 strains showed variation in the levels of methionine produced by individual transformants (i.e., transformants 1 and 2 produced approx. 1800 μM methionine when induced, whereas transformants 3 and 4 produced over 4000 μM methionine when induced). MA-699 strains, which express an S. coelicolor Hom variant, produced approximately 3000 μM methionine in the absence of IPTG. IPTG induction increased methionine production by 1500-2000 μM. These data show that expression of MetY (D231A) enhances methionine production. Methionine production was also enhanced in strain MA-699, relative to MA-622. Expression of MetY (D231 A) in strain MA-699 further enhanced methionine production in that strain.

A second variant allele of metY was expressed in C. glutamicum and assayed for its effect on methionine production. C. glutamicum strain MA-622 and strain MA-699 were transformed with a high copy plasmid, MB4239, that encodes MetY with a glycine to alanine mutation at position 232 (MetY (G232A)). The strains were cultured in the presence and absence of IPTG induction, and methionine productivity was assayed. The methionine production from each strain is plotted in FIG. 26. As shown, individual transformants of MA-622, when cultured under conditions in which expression of MetY (G232A) was induced, each produced over 1700 μM methionine. MA-699 strains produced approximately 3000 μM methionine in the absence of IPTG. IPTG induction resulted in an increased methionine production by 2000-3000 μM. These data show that expression of MetY (G232A) enhances methionine production. Methionine production was also enhanced in strain MA-699, relative to MA-622. Expression of MetY (G232A) in strain MA-699 further enhanced methionine production in that strain.

### Example 18

### Methionine Production in C. glutamicum Strains Expressing MetA and MetY Wild-Type and Mutant Alleles

Methionine production was assayed in five different C. glutamicum strains. Four of these strains express a unique combination of episomal C. glutamicum metA and metY alleles, as listed in Table 14. A fifth strain, MA-622, does not contain episomal metA or metY alleles. The amount of methionine produced by each strain (g/L) is listed in Table 21.

The highest levels of methionine production were observed in strains expressing a combination of either a wild-type metA and a variant metY, or a wild-type metY and a variant metA.

TABLE 21
Methionine production in strains expressing C. glutamicum metA and
metY wild-type and mutant alleles

| | | | | methionine |
|---|---|---|---|---|
| Strain | IPTG | metA allele | metY allele | (g/L) |
| MA-622 | − | None | none | 0.00 |
| MA-641 | − | WT | WT | 0.03 |
| MA-721 | − | K233A | WT | 0.00 |
| MA-721 | + | K233A | WT | 0.53 |
| MA-725 | − | WT | D231A | 0 |
| MA-725 | + | WT | D231A | 0.28 |
| MA-727 | − | WT | G232A | 0 |
| MA-727 | + | WT | G232A | 0.37 |

### Example 19

### Combinations of Genetic Manipulations, Using Both Heterologous and Native Genes, Elicits Production of Aspartate-Derived Amino Acids

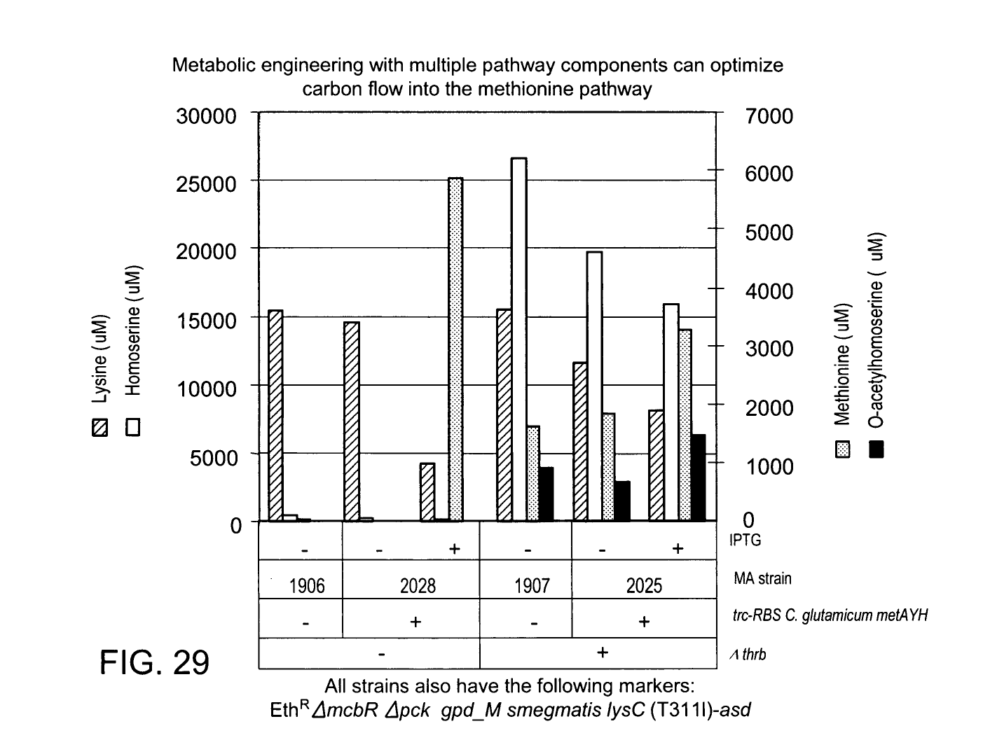

As described above, gene combinations may optimize corynebacteria for the production of aspartate-derived amino acids. Below are examples that show how multiple manipulations can increase the production of methionine. FIG. 29 shows the production of several aspartate-derived amino acids by strains MA-2028 and MA-2025 along with titers from their parent strains MA-1906 and MA-1907, respectively. MA-1906 was constructed by using plasmid MB4276 to delete the native pck locus in MA-0622 and replace pck with a cassette for constitutive expression of the M. smegmatis lysC(T311I)-asd operon. MA-1907 was generated by similar transformation of MB4276 into MA-0933. MA-2028 and MA-2025 were constructed by transformation of the respective parents with MB4278, an episomal plasmid for inducible expression of a synthetic C. glutamicum metAYH operon (see Example 3). Parent strains MA-1906 and MA-1907 produce lysine or lysine and homoserine, respectively; methionine and methionine pathway intermediates are also produced by these strains. The scale for lysine and homoserine is on the left y-axis; the scale for methionine and O-acetylhomoserine is on the right y-axis. With IPTG induction, MA-2028 showed a decrease in lysine levels and an increase in methionine levels. MA-2025 also displayed an IPTG-dependent decrease in lysine production, together with increased production of methionine and O-acetylhomoserine.

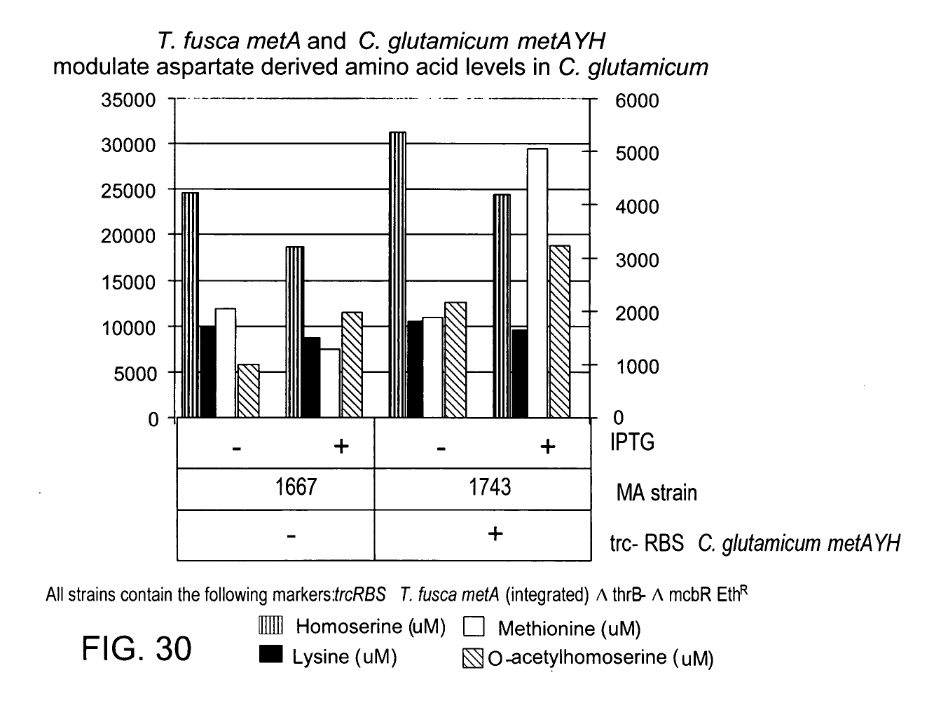

Strain MA-1743 is another example of how combinatorial engineering can be employed to generate strains that produce methionine. MA-1743 was generated by transformation of MA-1667 with metAYH expression plasmid MB4278. MA-1667 was constructed by first engineering strain MA-0422 (see Example 15) with plasmid MB4084 to delete thrB, and next using plasmid MB4286 to both delete the mcbR locus and replace mcbR with an expression cassette containing trcRBS-T. fusca metA. In this example and in other examples where trcRBS has been integrated at single copy, expression does not appear to be as tightly regulated as seen with the episomal plasmids (as judged by amino acid production). This may be due to decreased levels of the laclq inhibitor protein. IPTG induction of strain MA-1743 elicits production of methionine and pathway intermediates, including O-acetylhomoserine (FIG. 30; the scale for lysine and homoserine is on the left y-axis; the scale for methionine and O-acetylhomoserine is on the right y-axis).

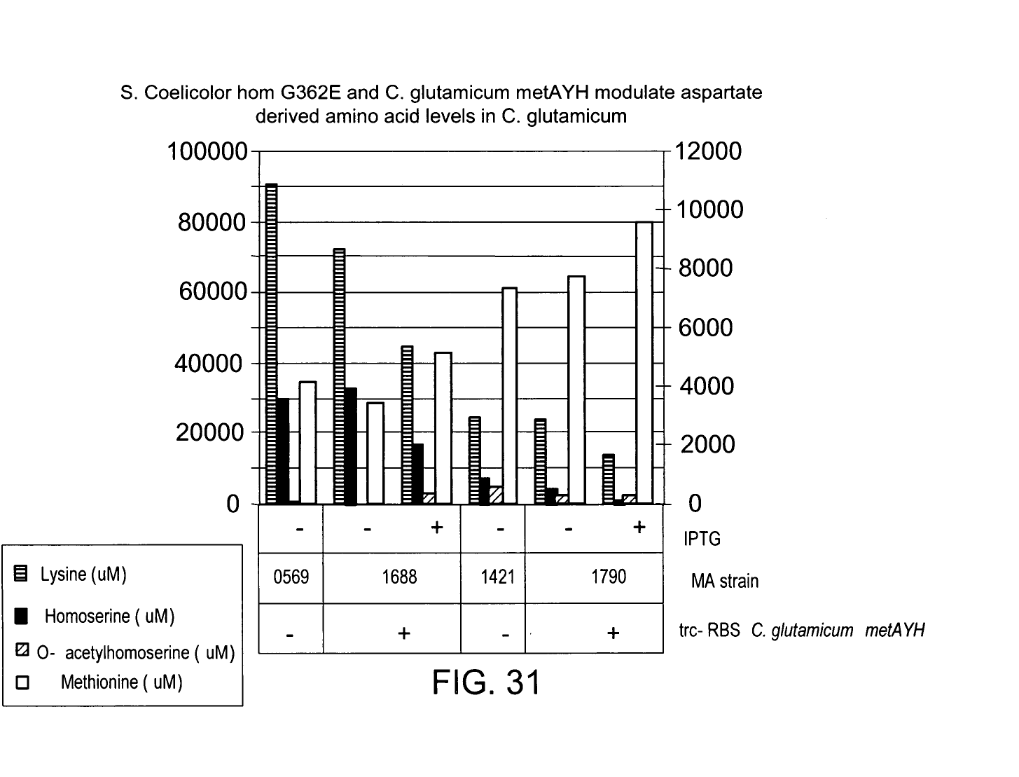

Strains MA-1688 and MA-1790 are two additional strains that were engineered with multiple genes, including the MB4278 metAYH expression plasmid (see FIG. 31; the scale for lysine and homoserine is on the left y-axis; the scale for methionine and O-acetylhomoserine is on the right y-axis). Transforming MA-0569 with MB4278 generated MA-1688. MA-0569 was constructed by sequentially using MB4192 and MB4165 to first delete the hom-thrB locus and integrate the gpd-S. coelicolor hom(G362E) expression cassette and then delete mcbR. MA-1790 construction required several steps. First, a NTG mutant derivative of MA-0428 was identified based on its ability to allow for growth of a Salmonella metE mutant. In brief, a population of mutagenized MA-0428 cells was plated onto a minimal medium containing threonine and a lawn (>10⁶ cells of the Salmonella metE mutant). The Salmonella metE mutant requires methionine for growth. After visual inspection, the corynebacteria colonies (e.g. MA-0600) surrounded by a halo of Salmonella growth were isolated and subjected to shake flask analysis. Strain MA-600 was next mutagenized to ethionine resistance as described above, and one resulting strain was designated MA-0993. The mcbR locus was then deleted from MA-0993 using plasmid MB4165, and MA-1421 was the product of this manipulation. Transformation of MA-1421 with MB4278 generated MA-1790. FIG. 31 shows that IPTG induction stimulates methionine production in both MA-1688 and MA-1790, and decreases in lysine and homoserine titers.

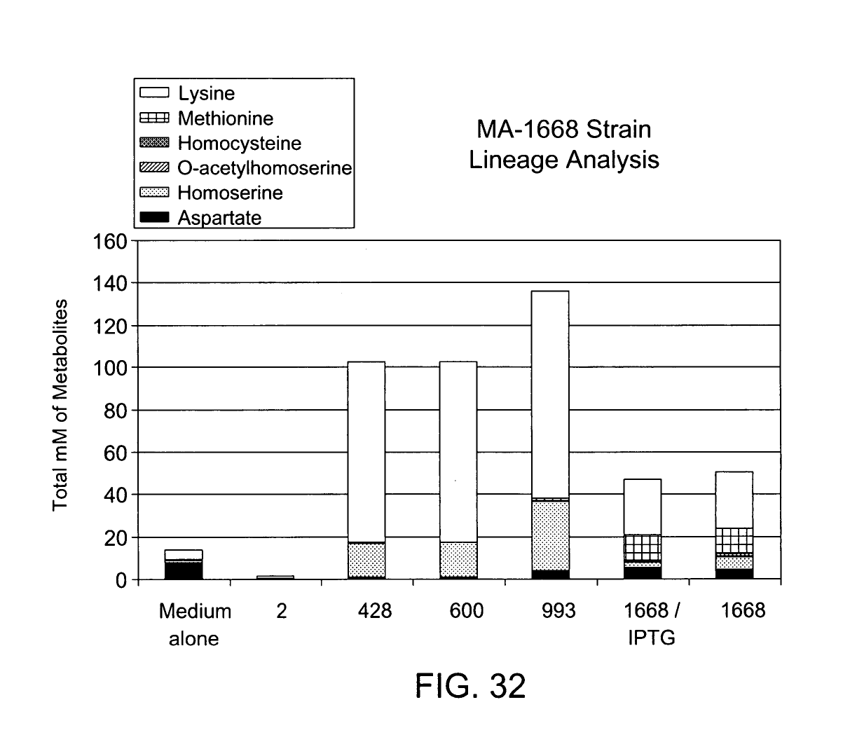

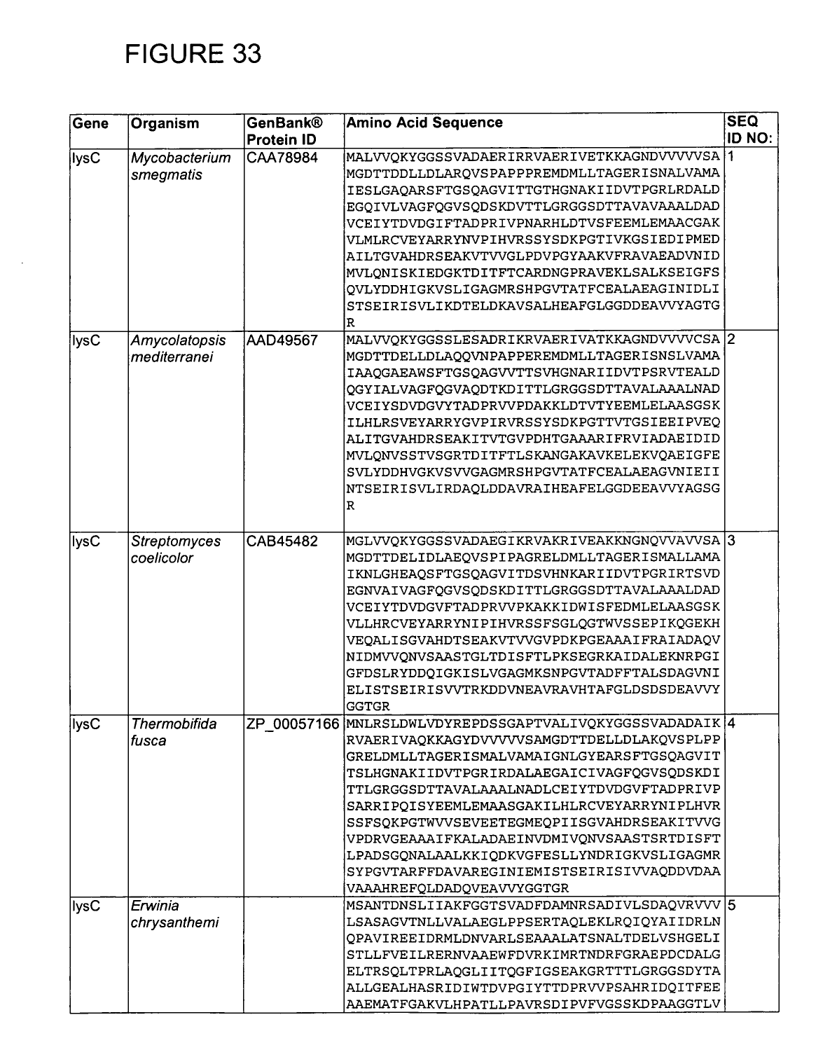

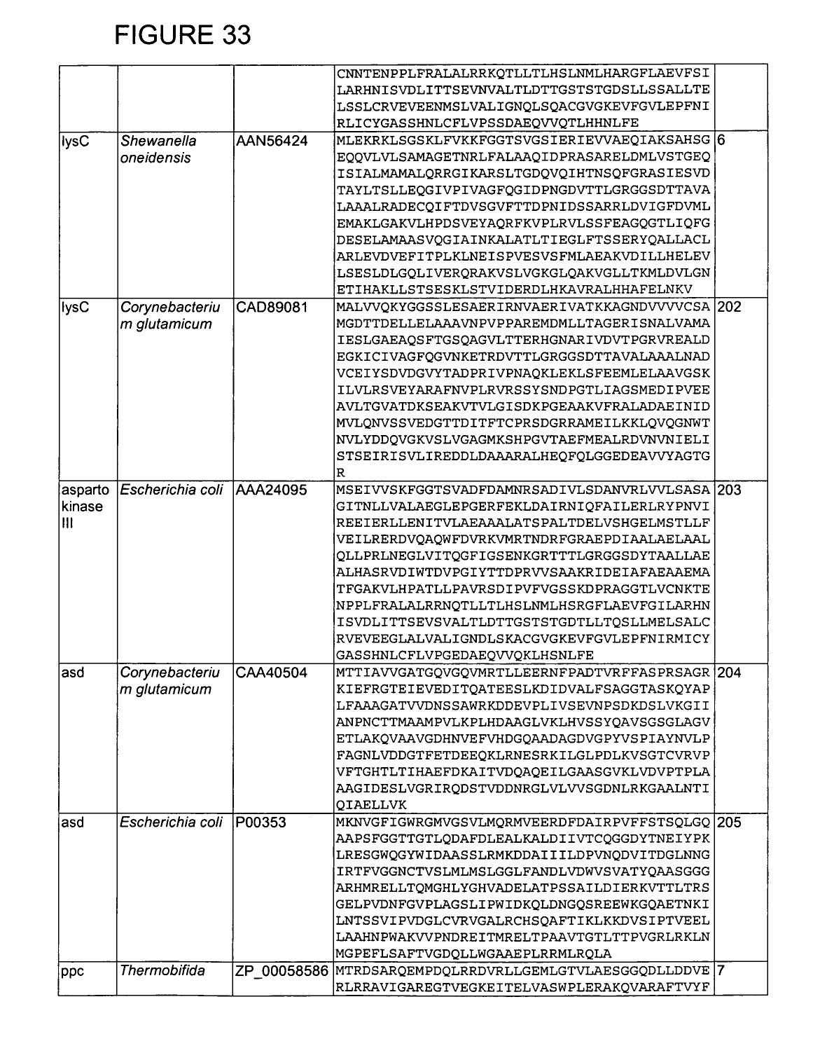

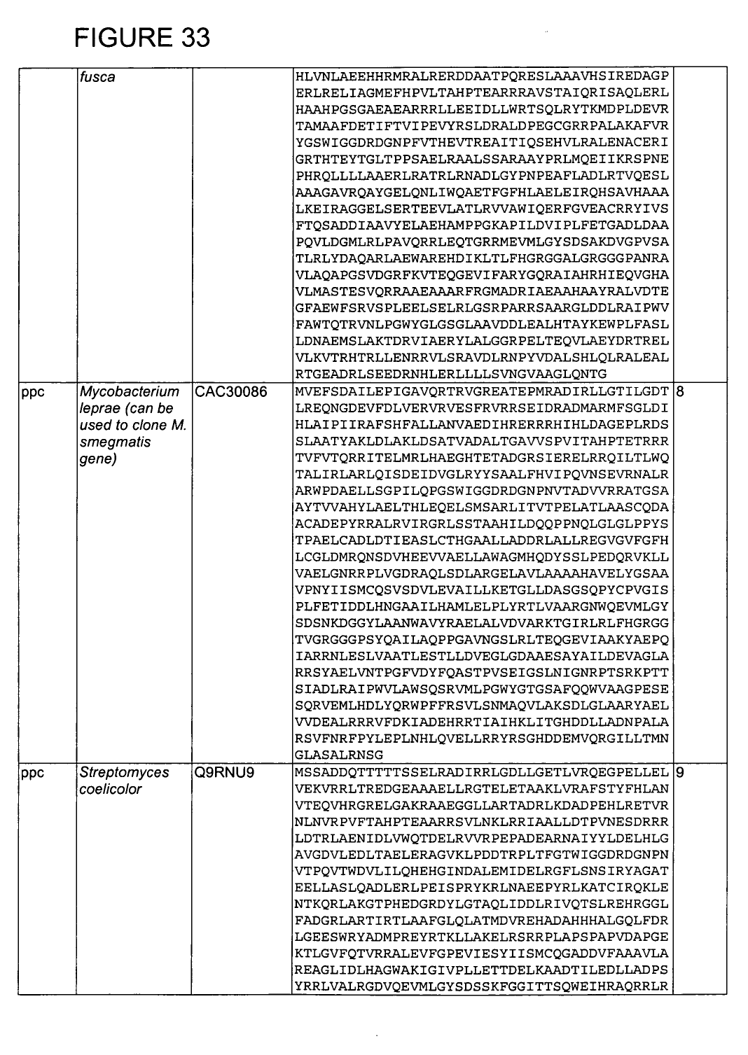

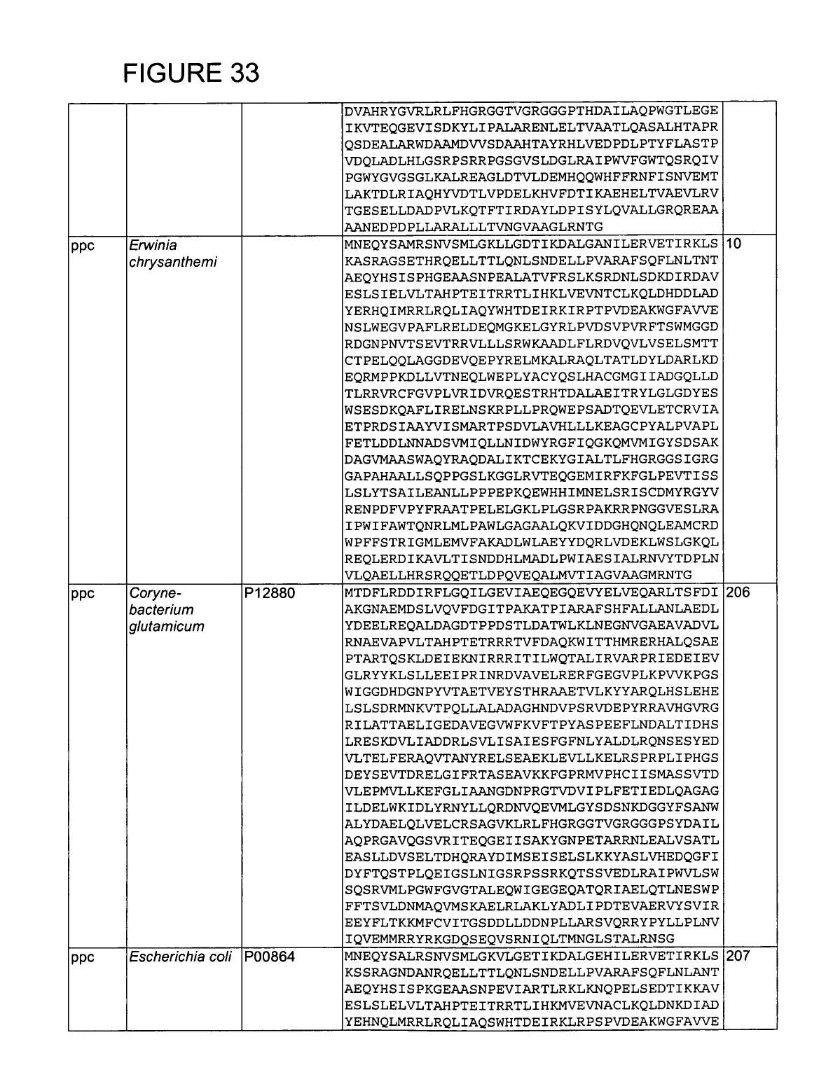

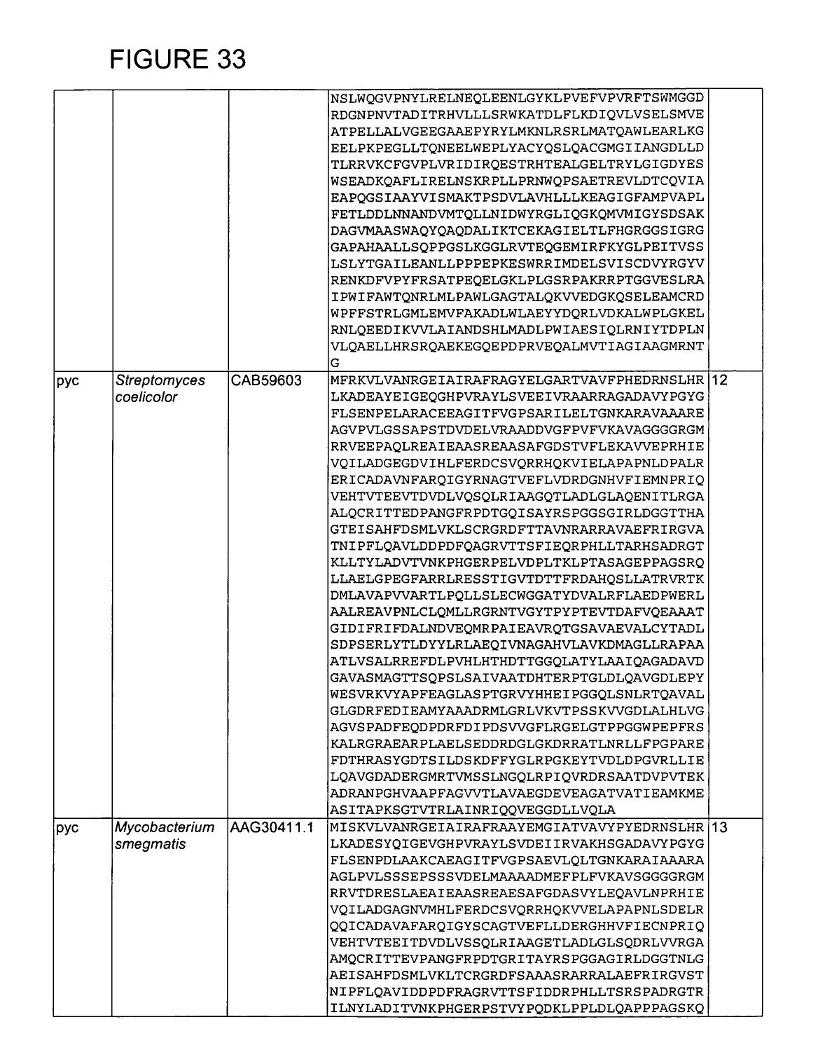

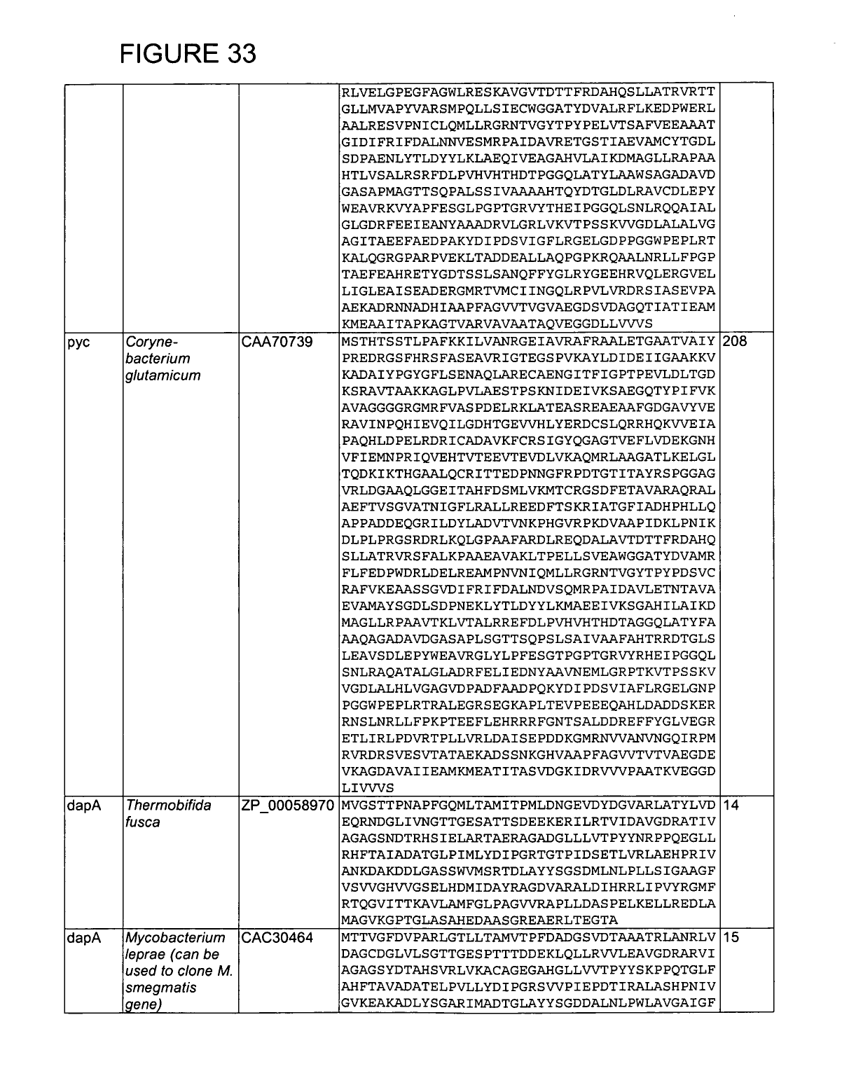

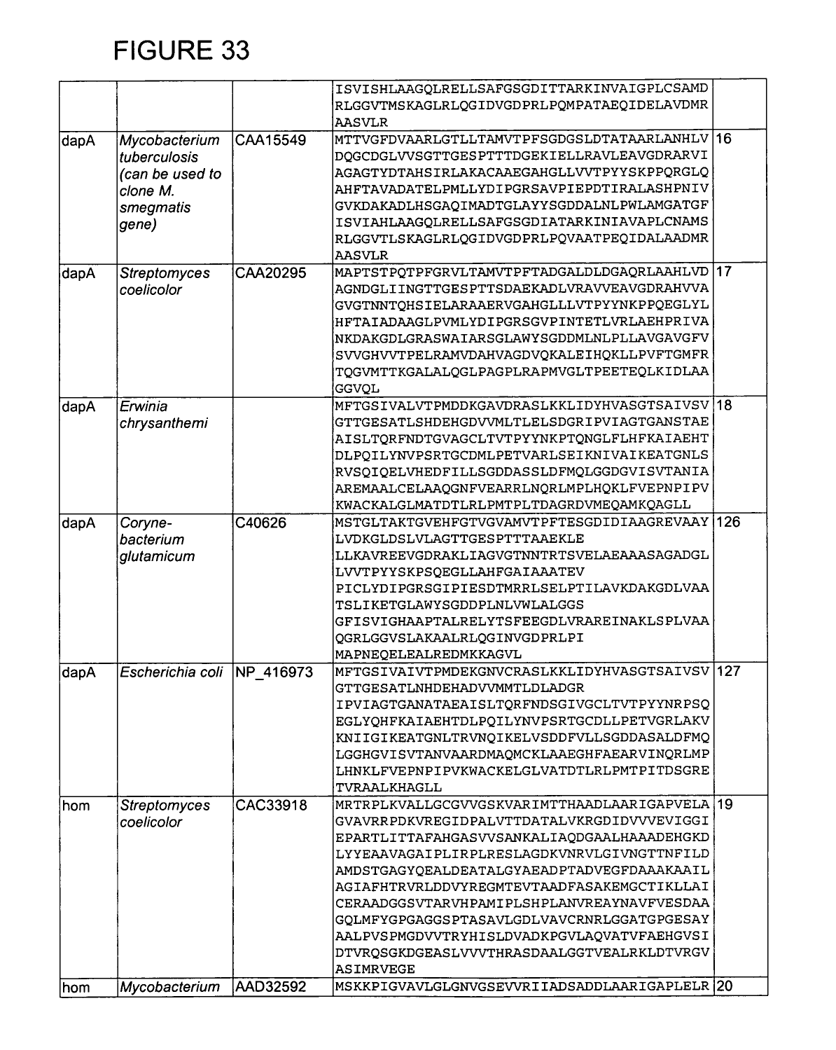

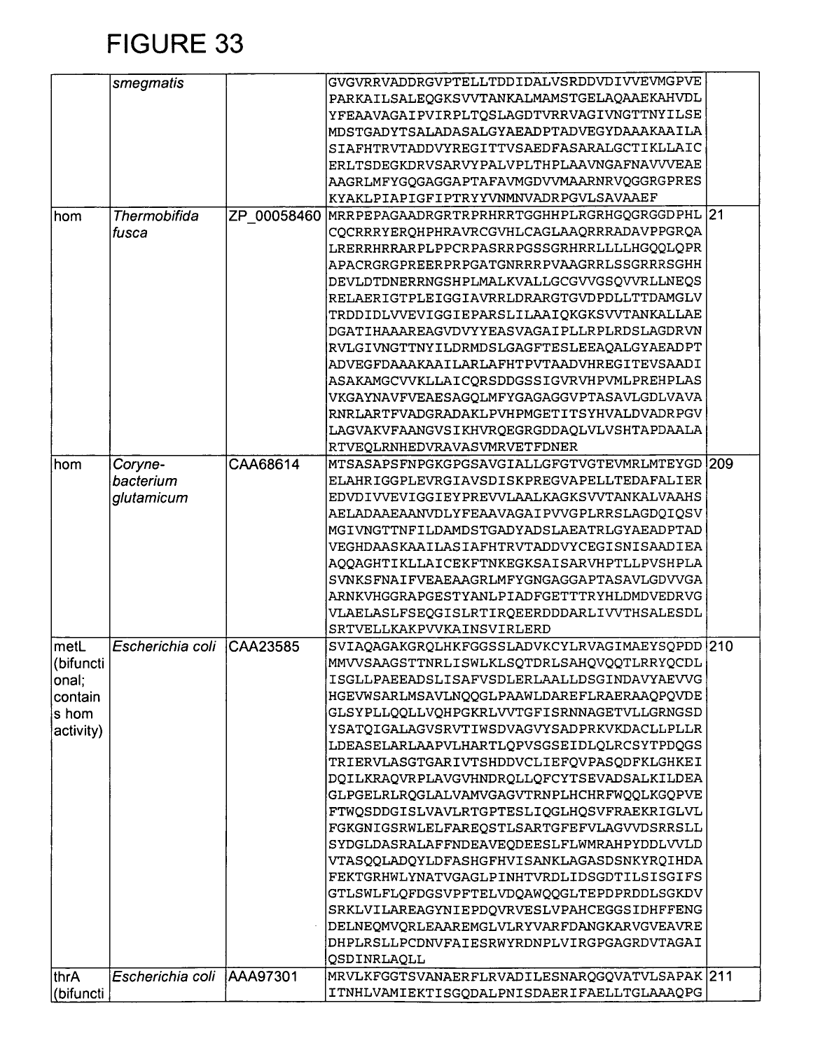

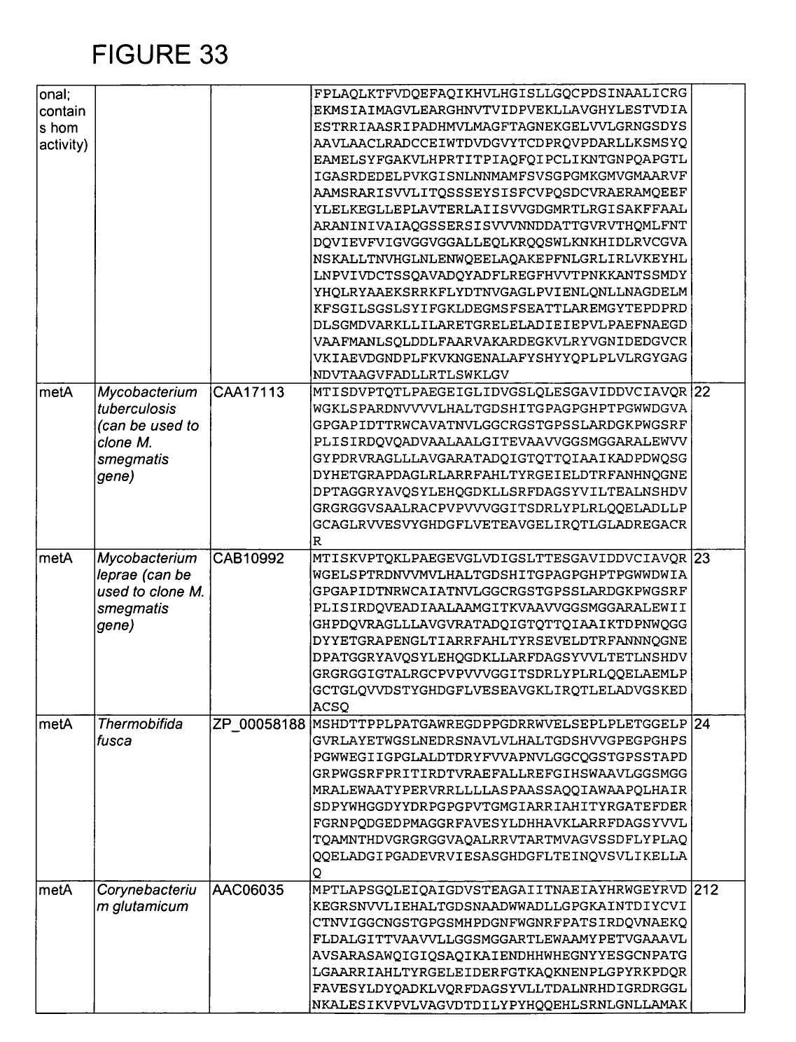

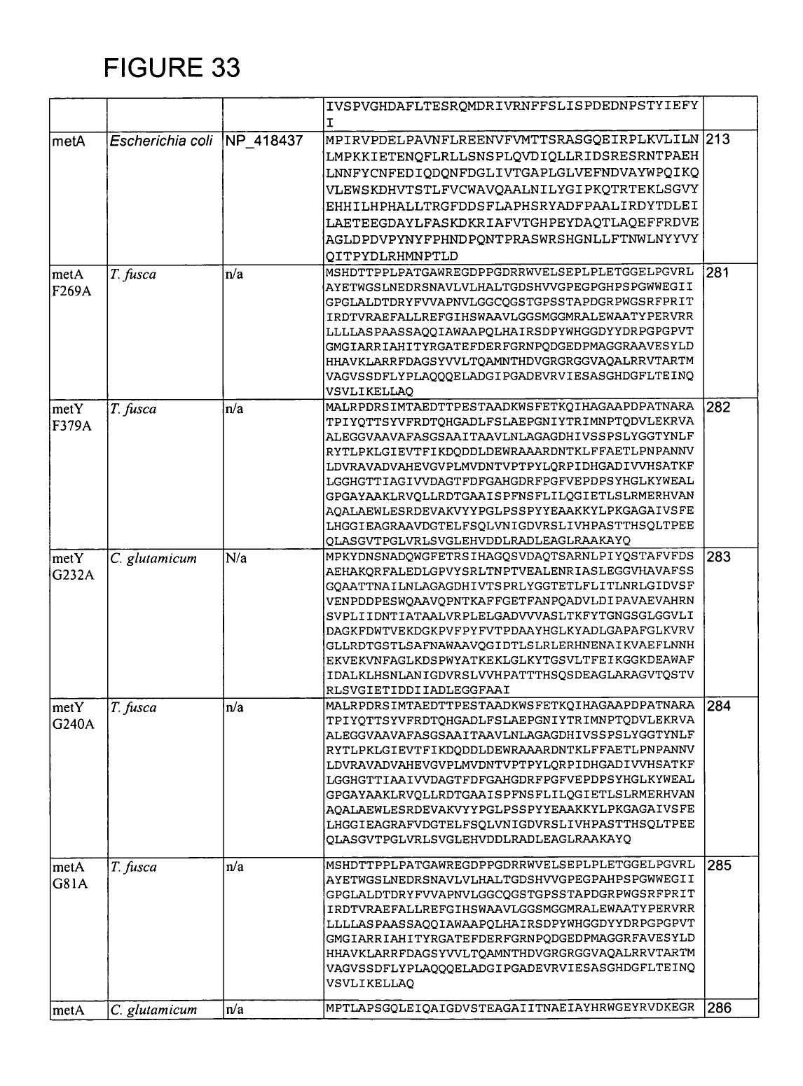

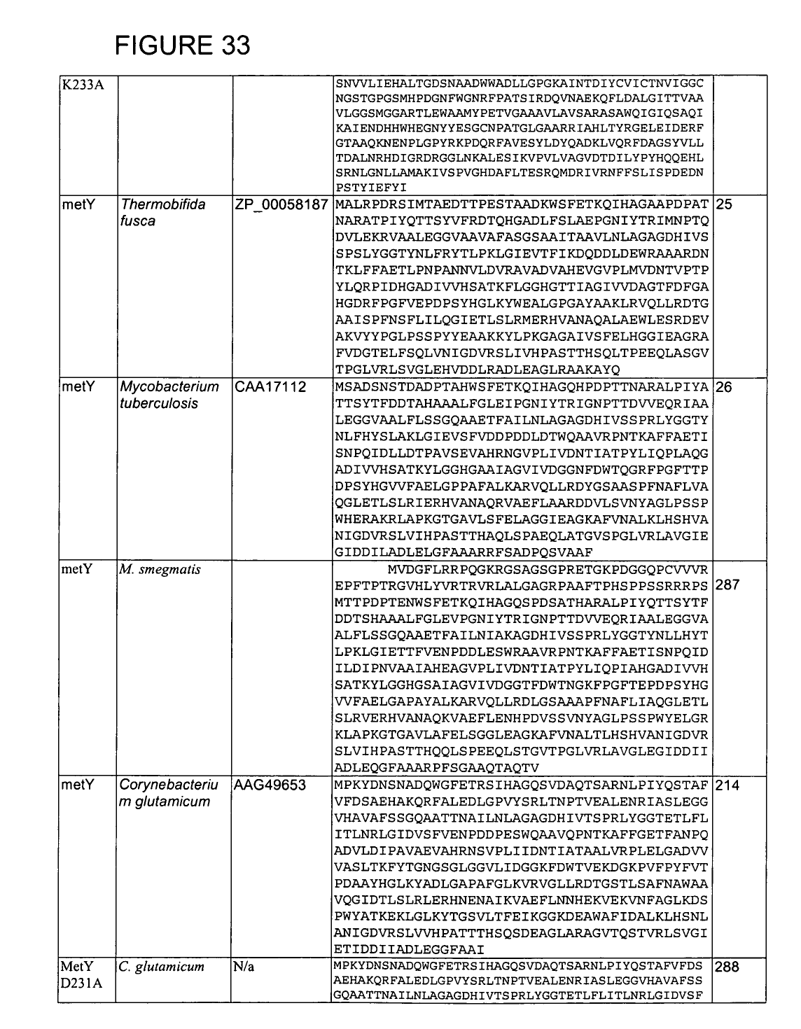

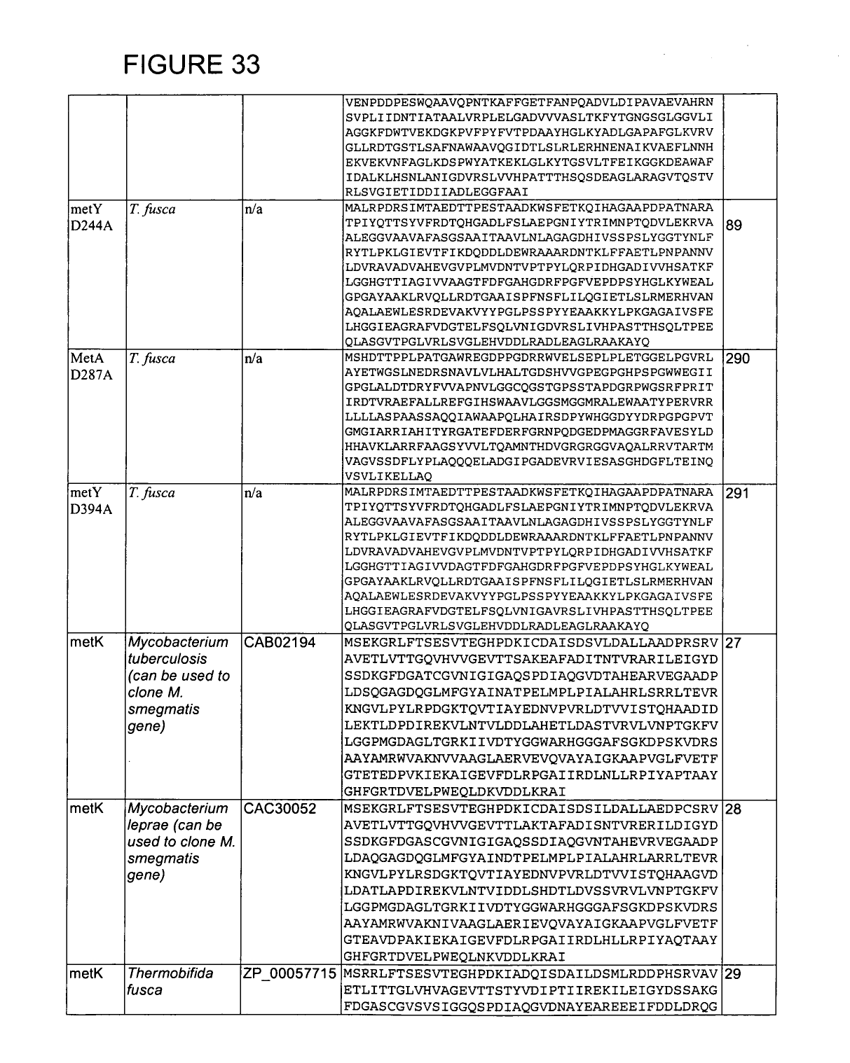

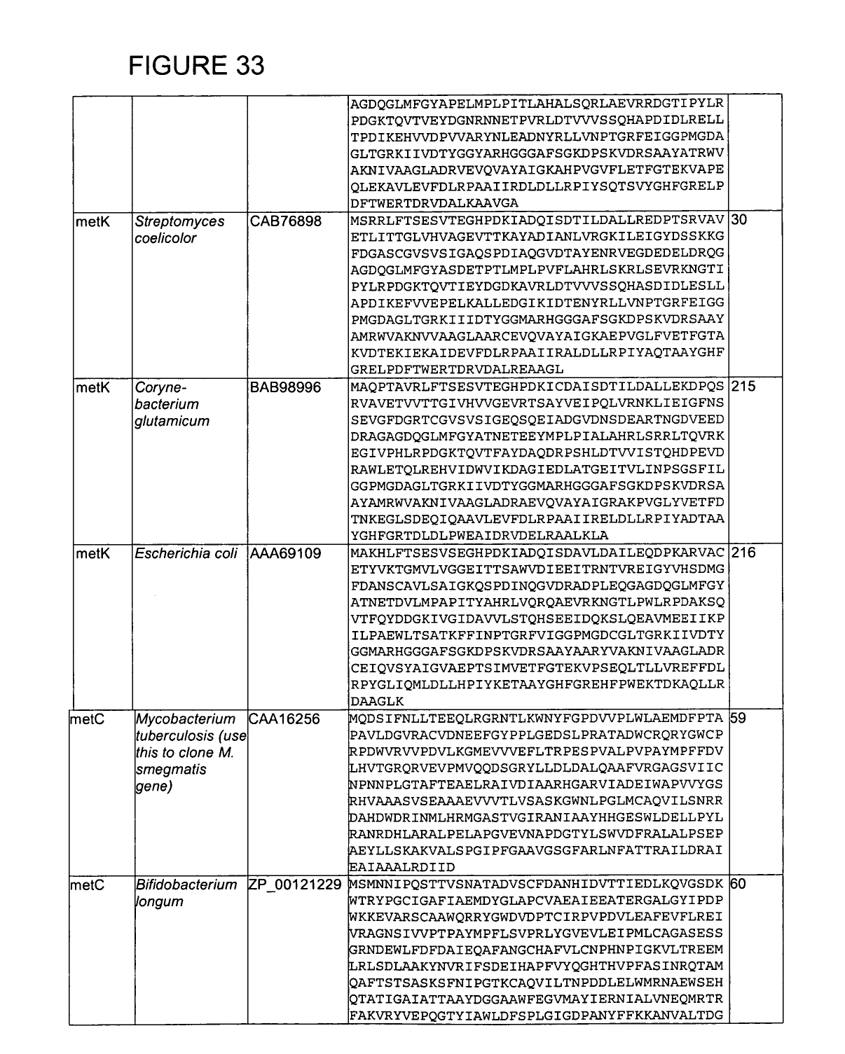

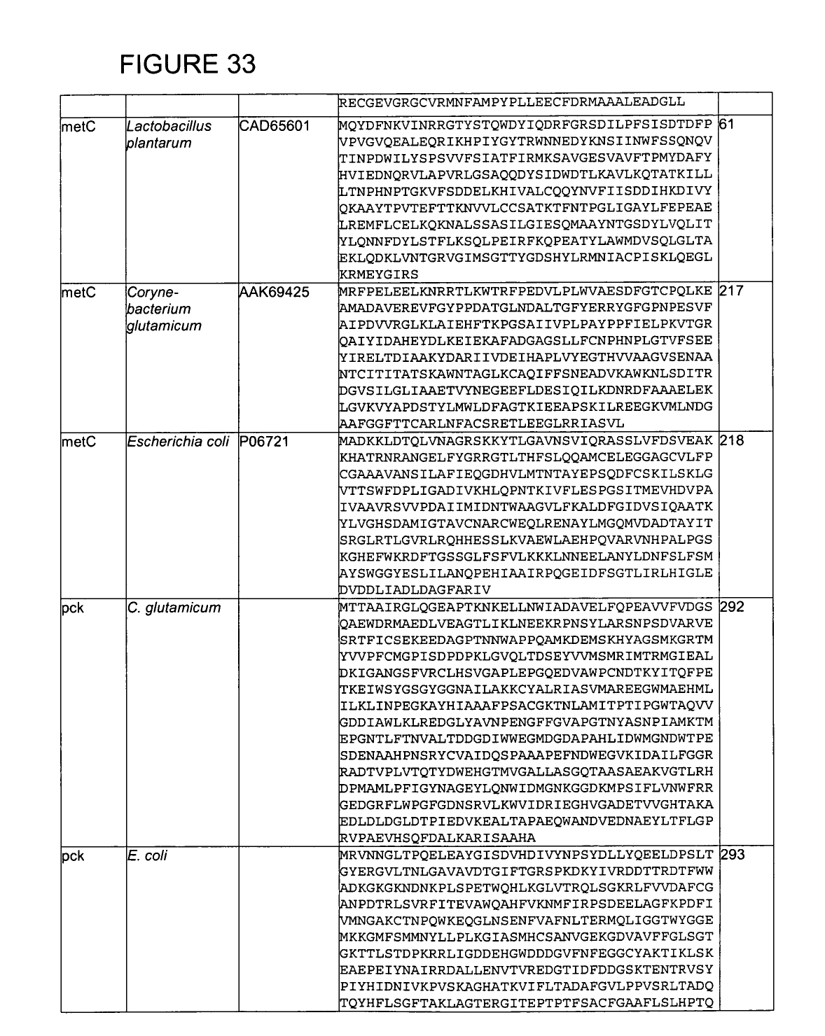

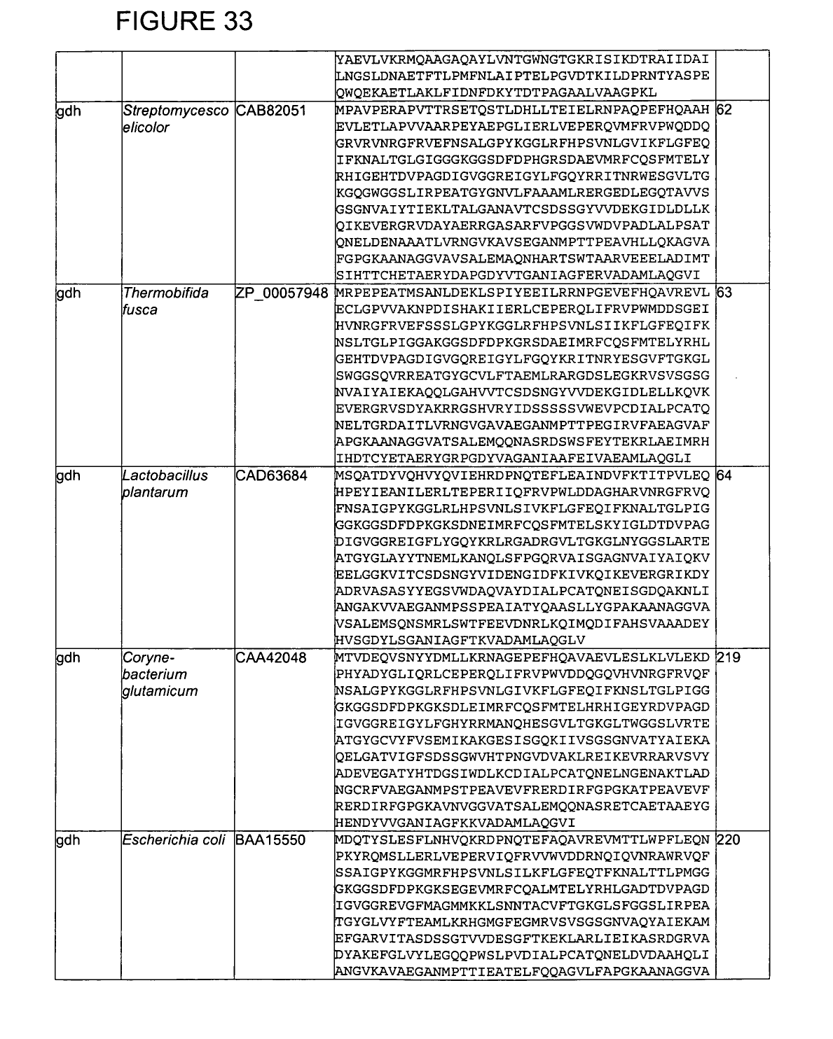

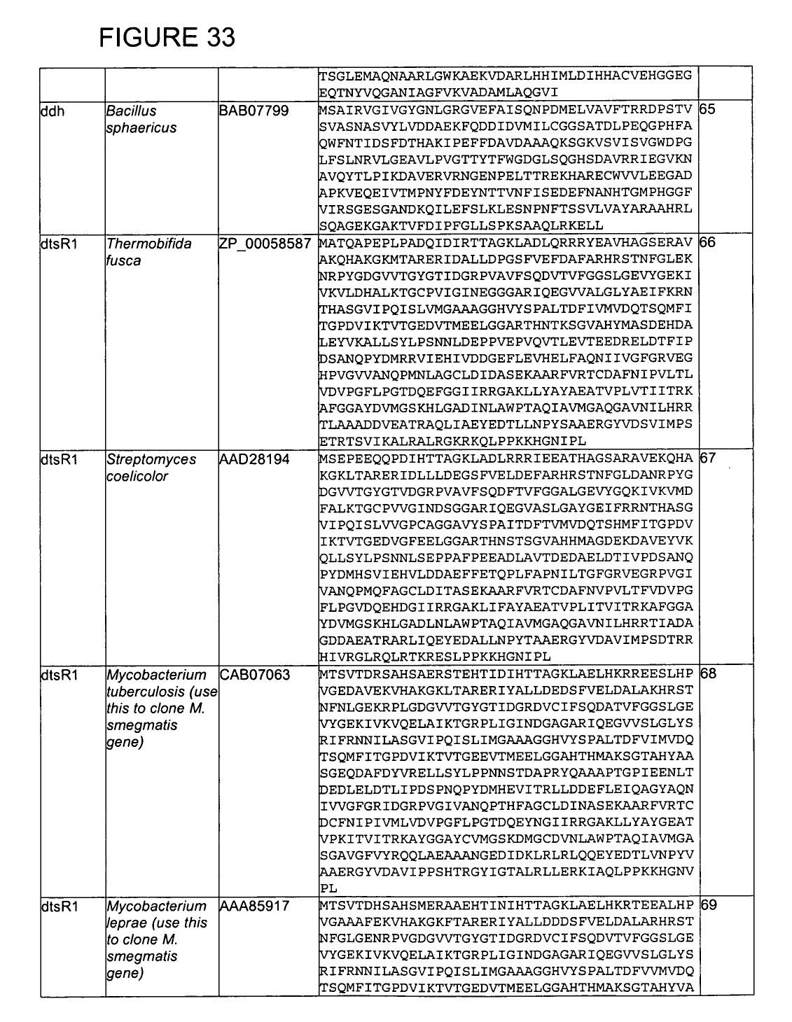

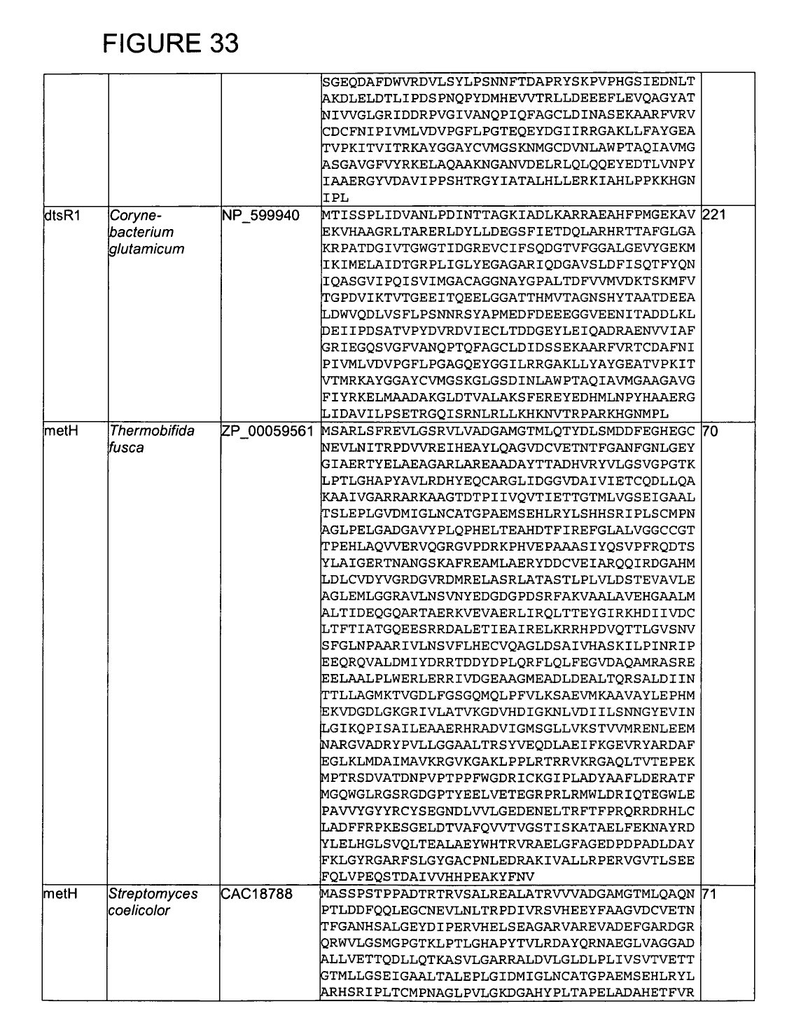

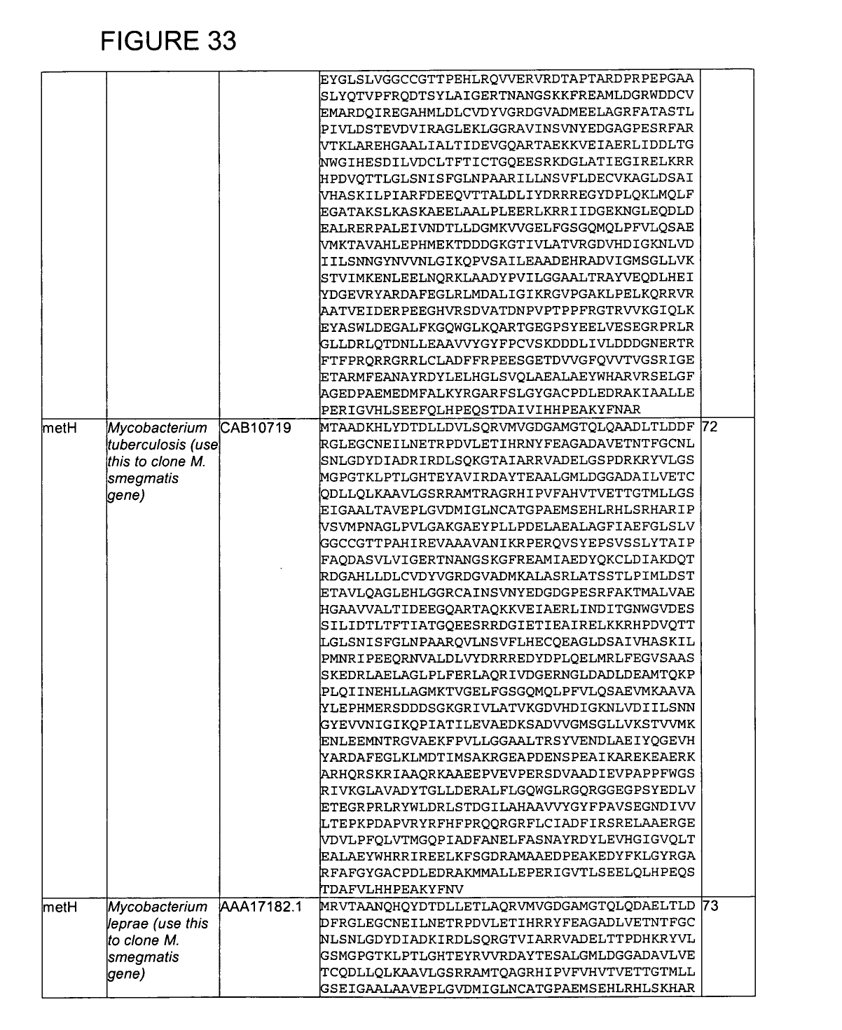

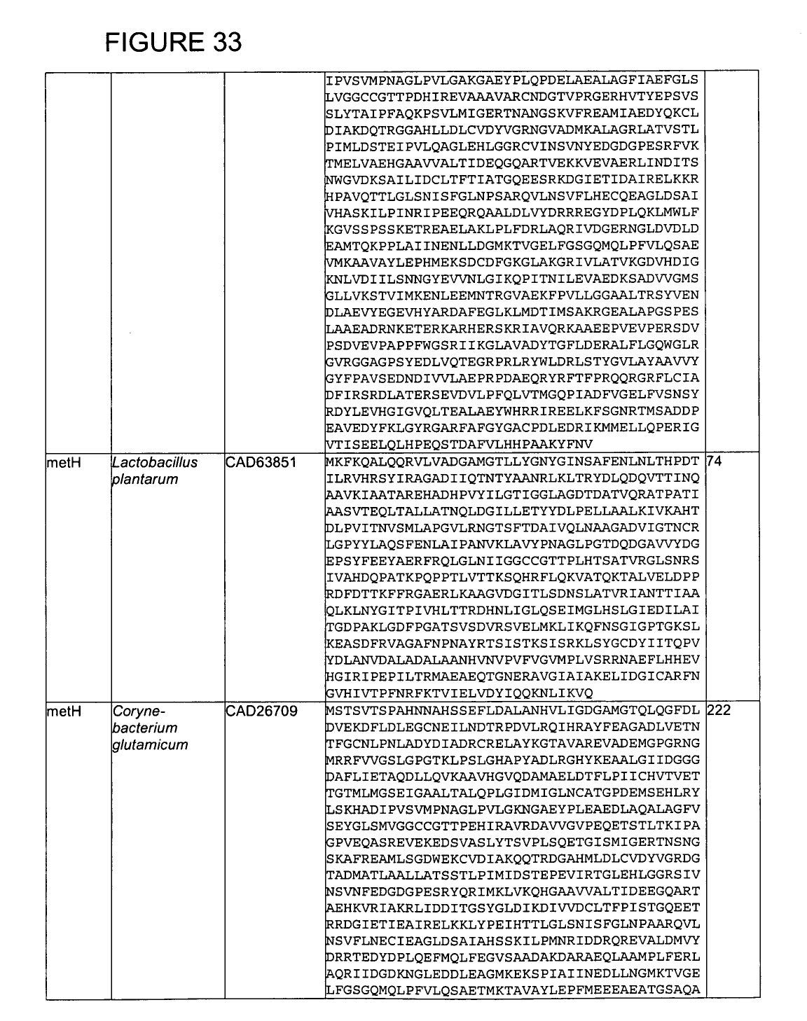

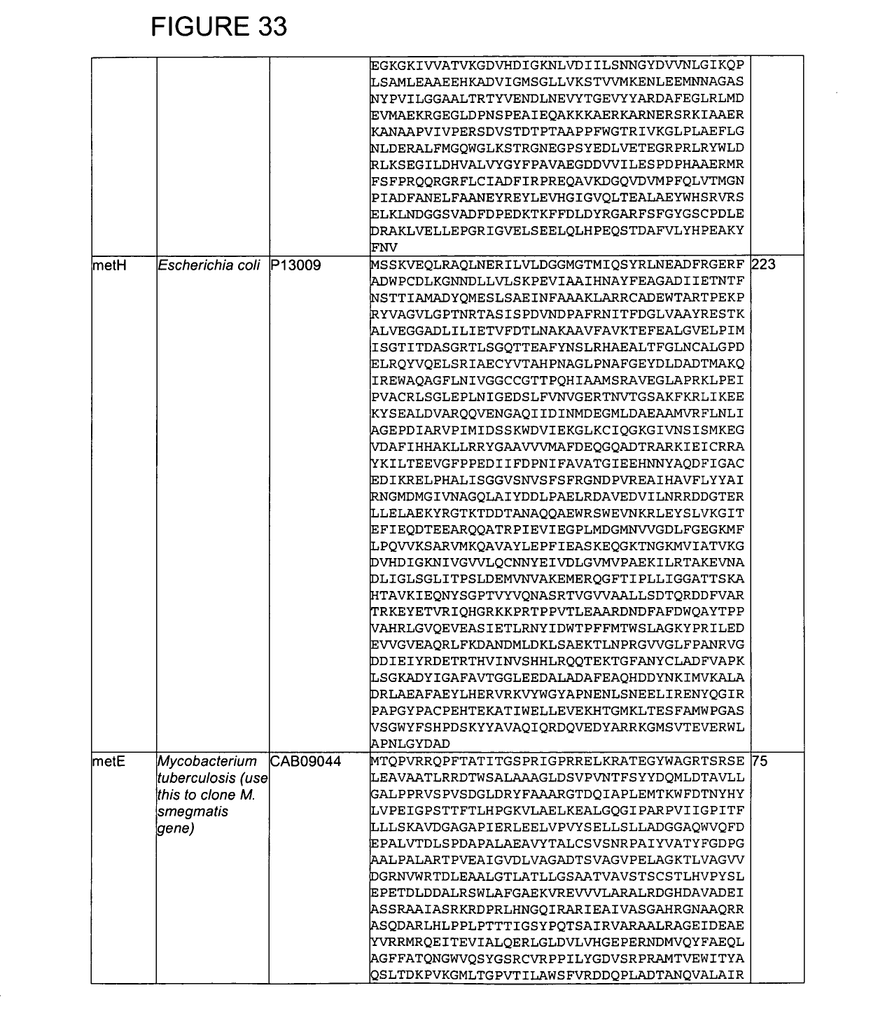

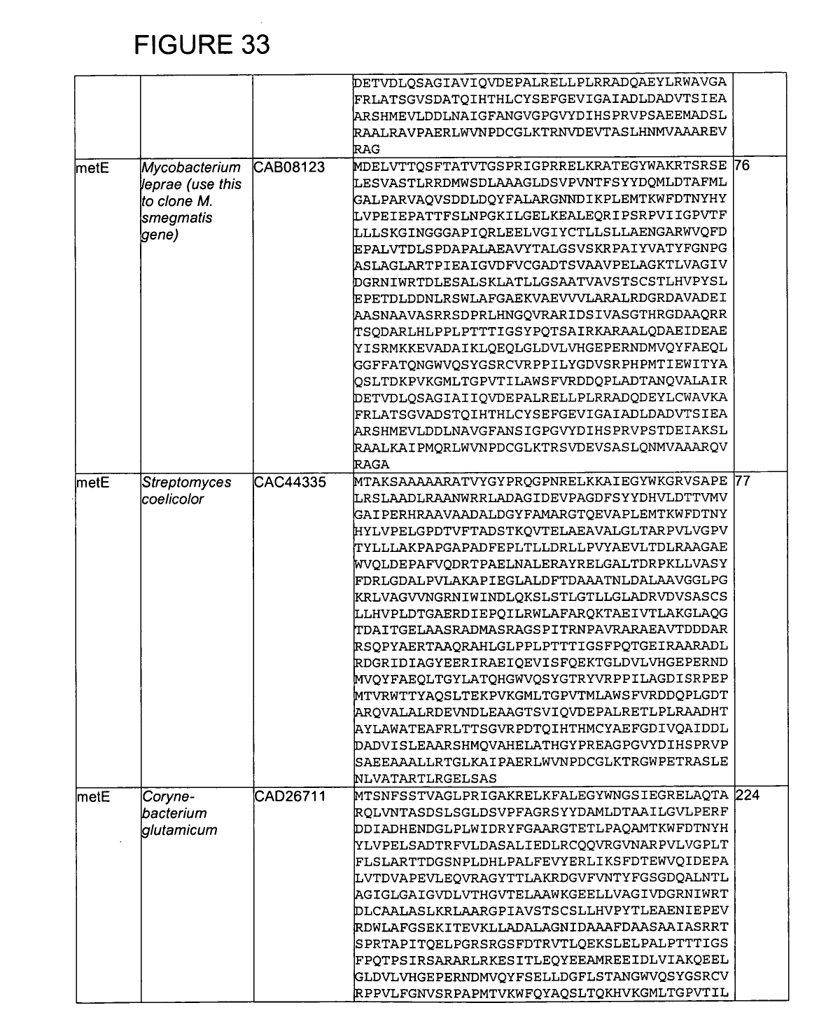

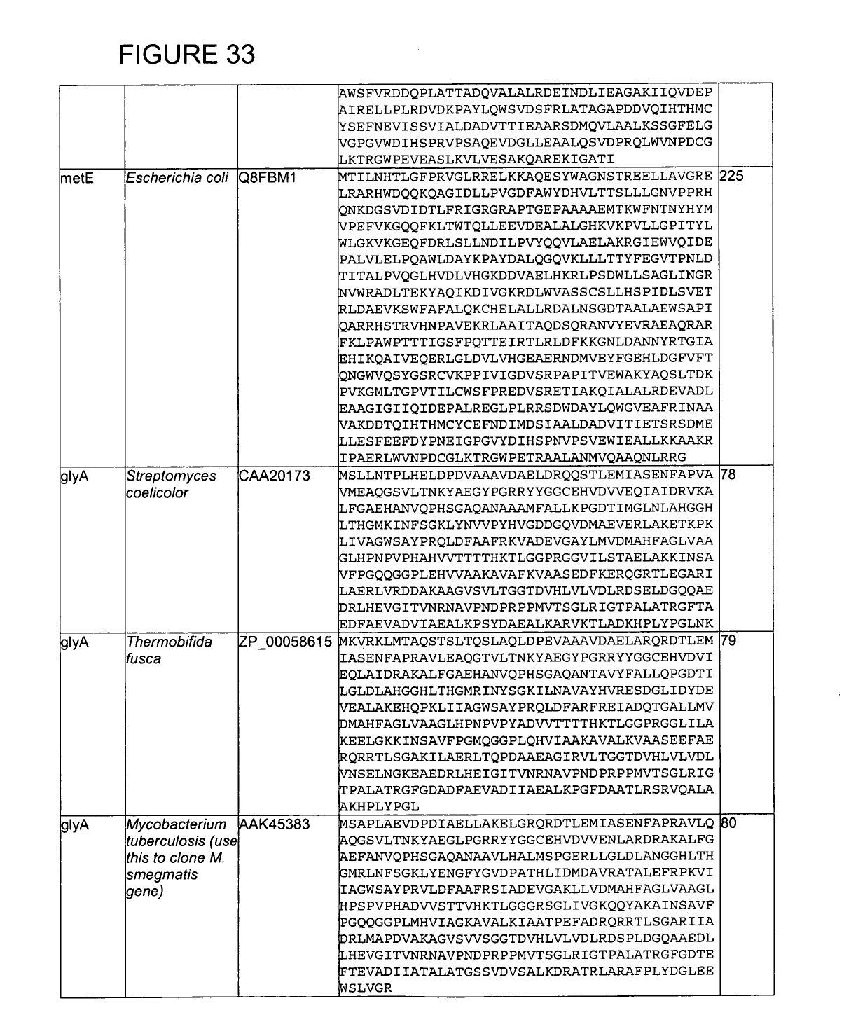

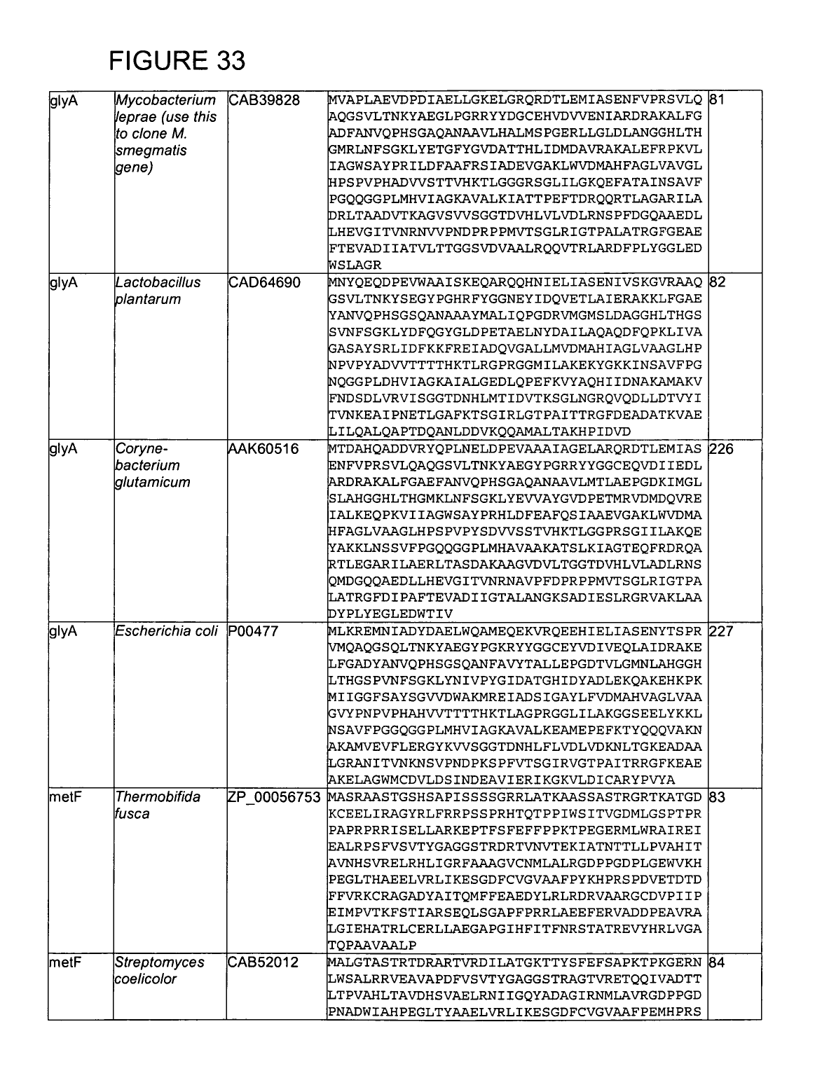

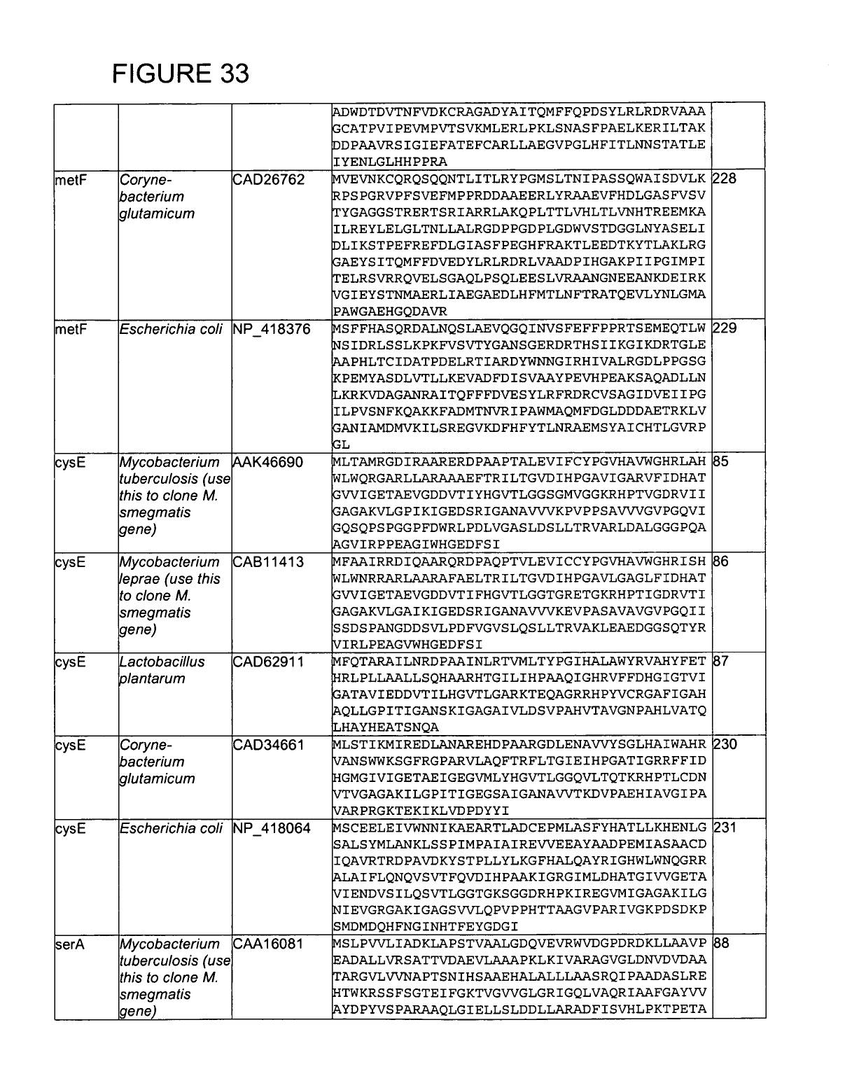

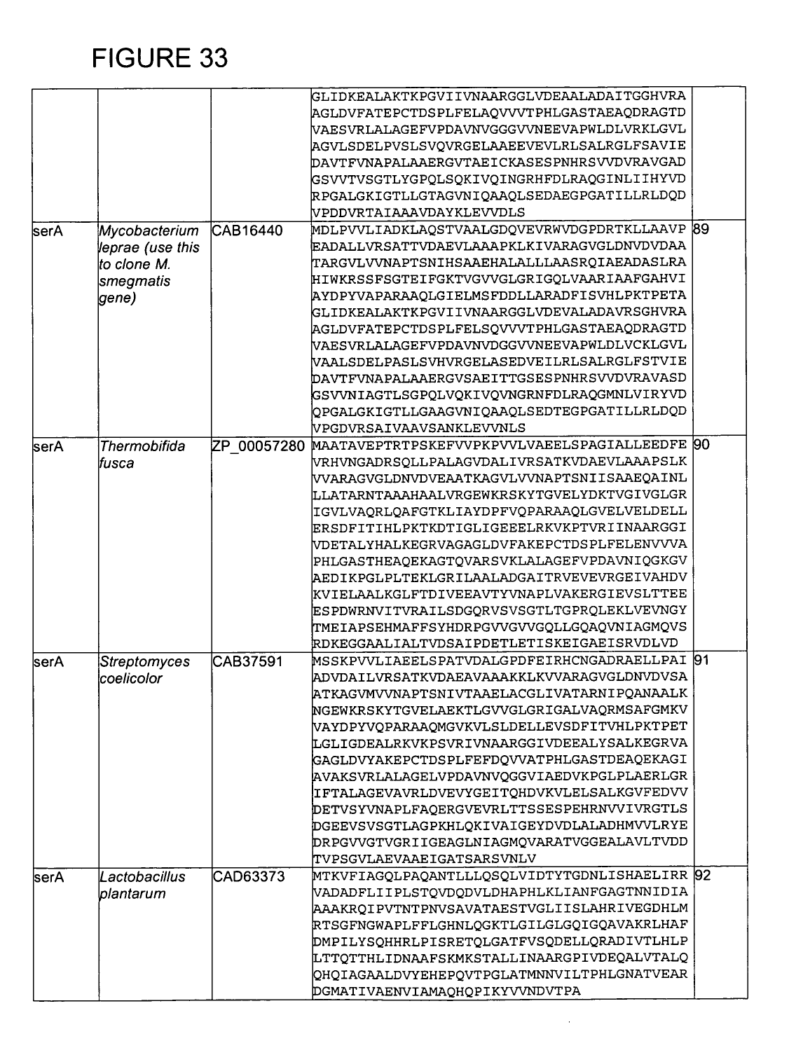

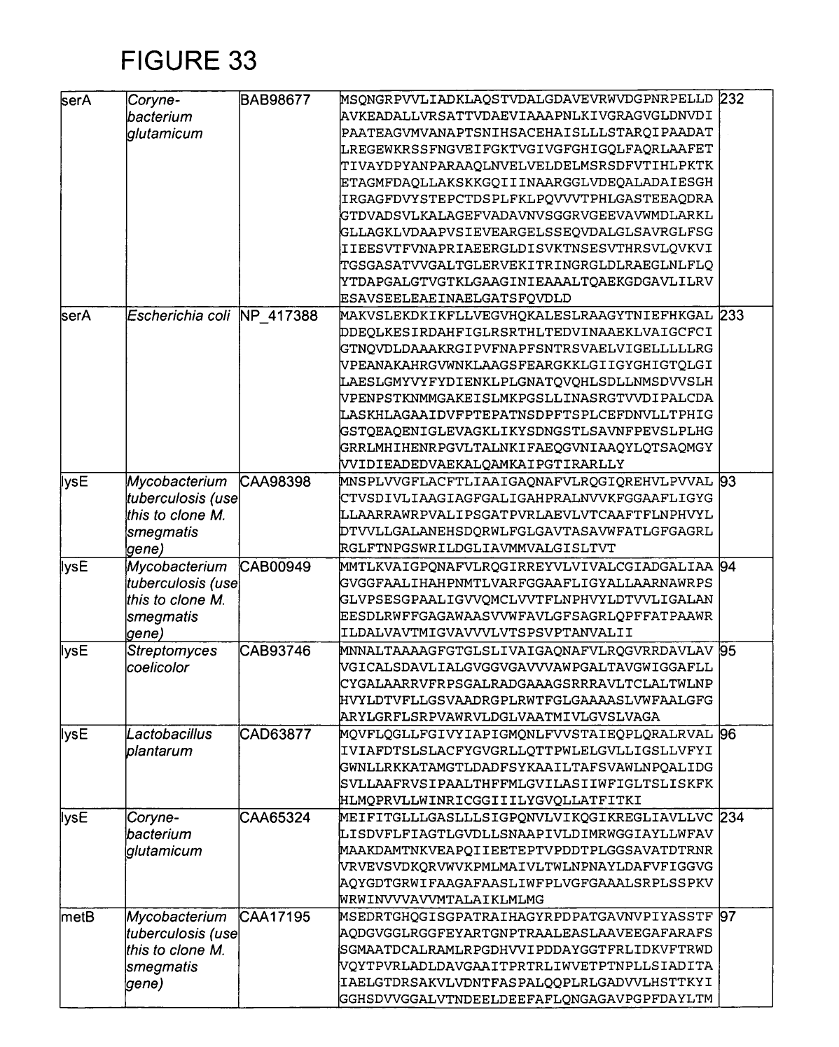

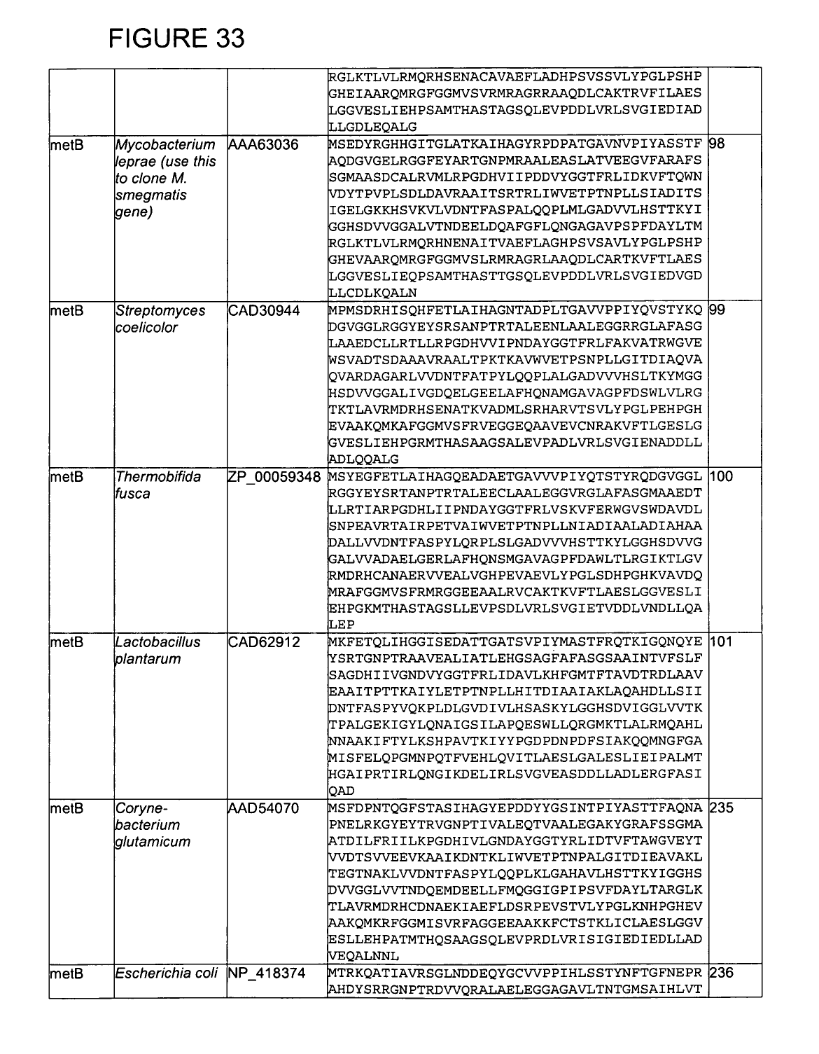

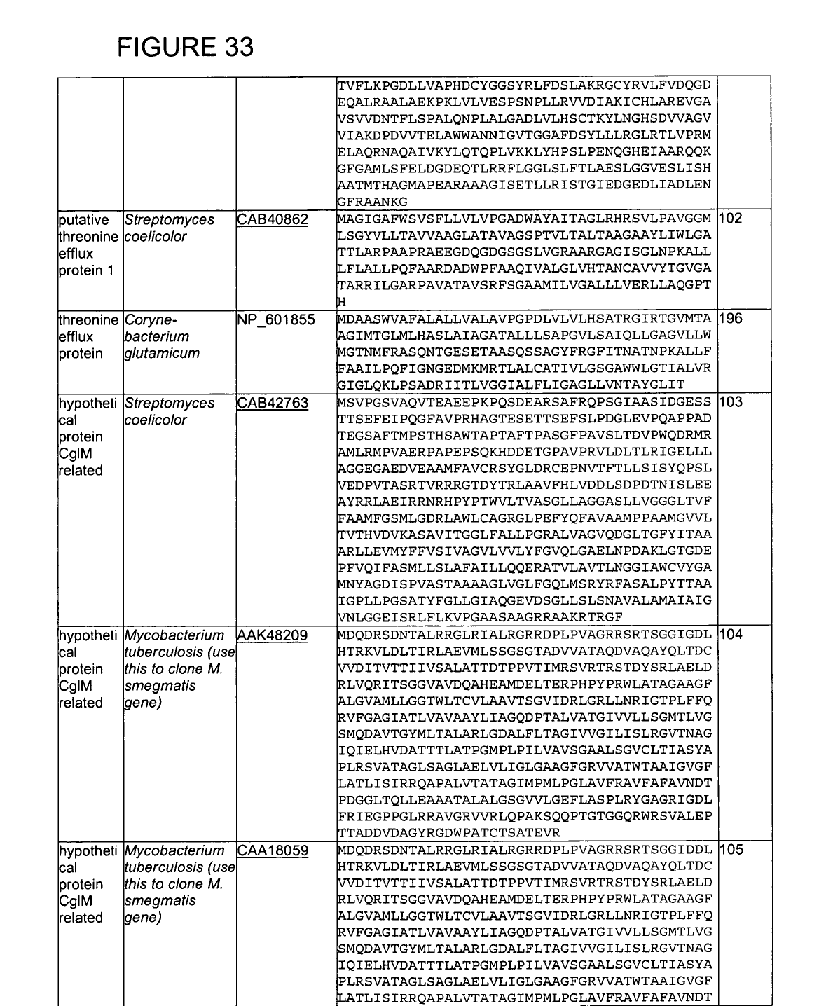

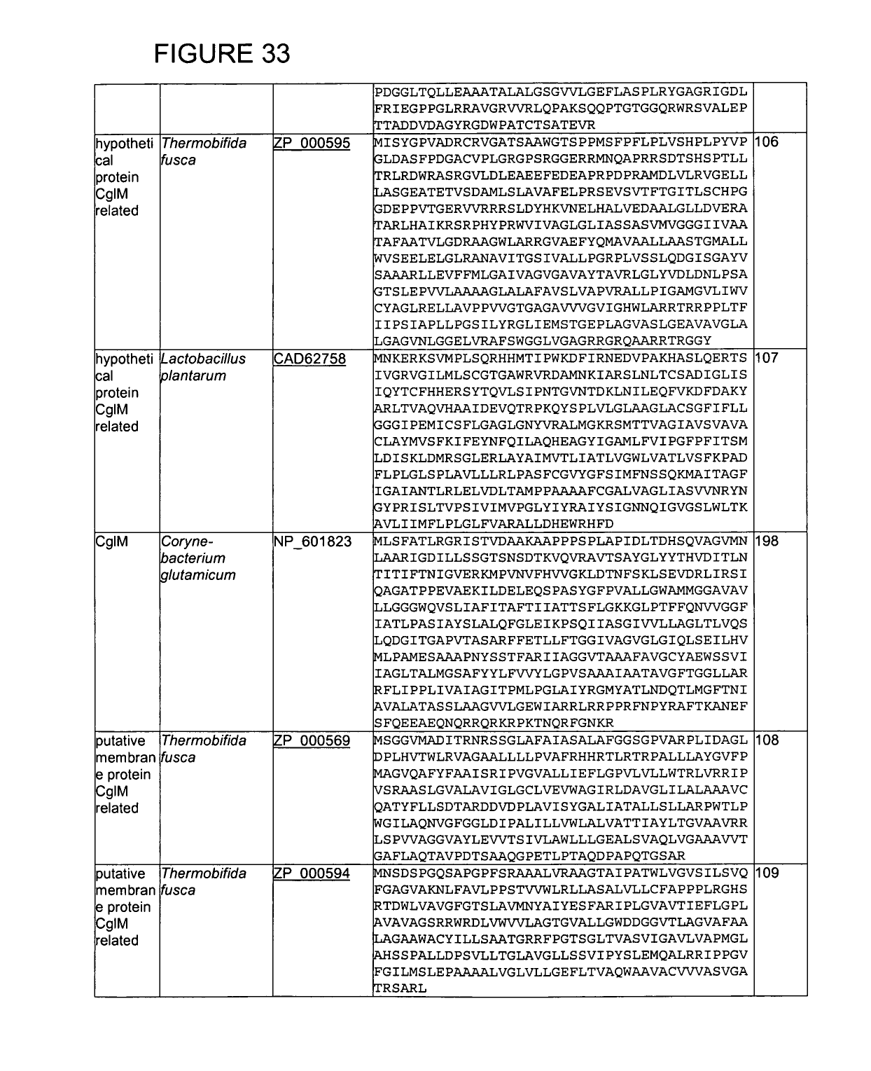

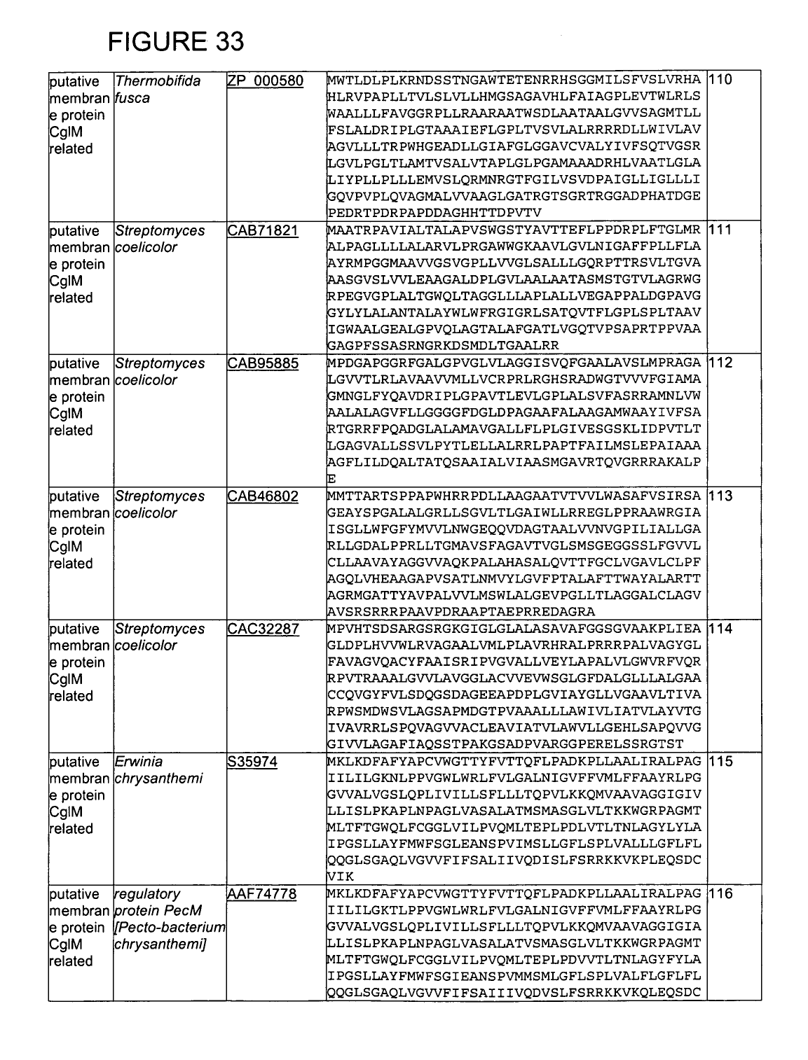

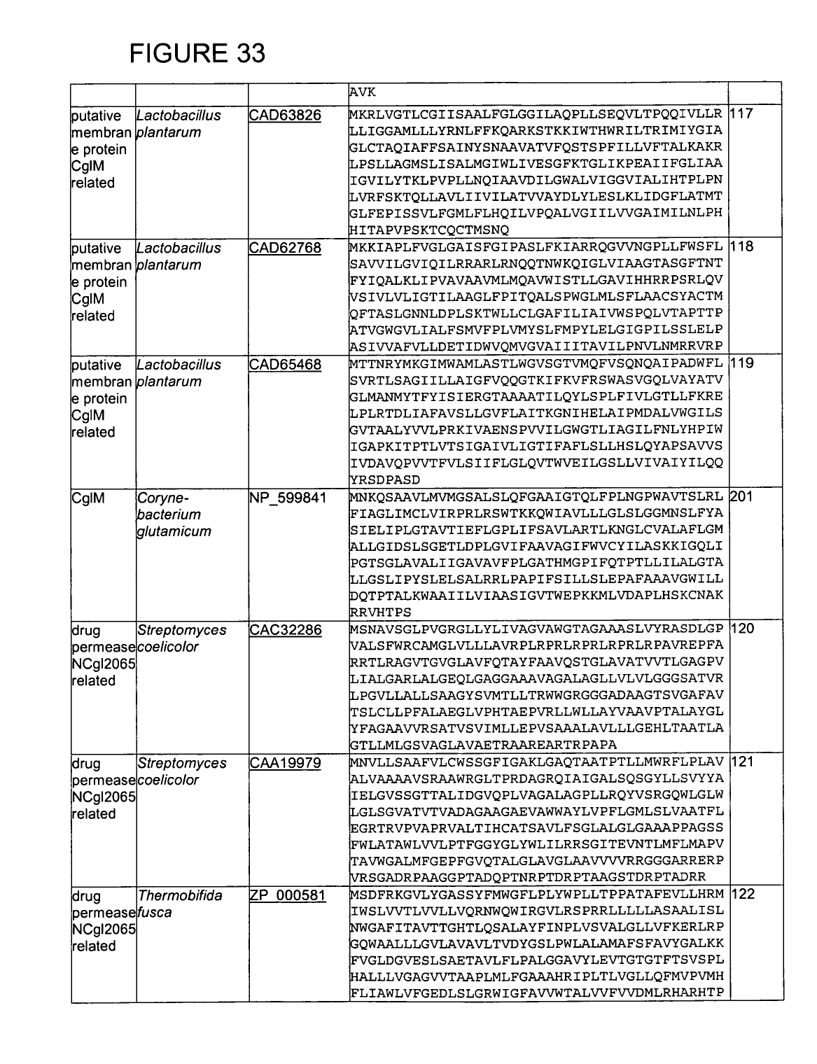

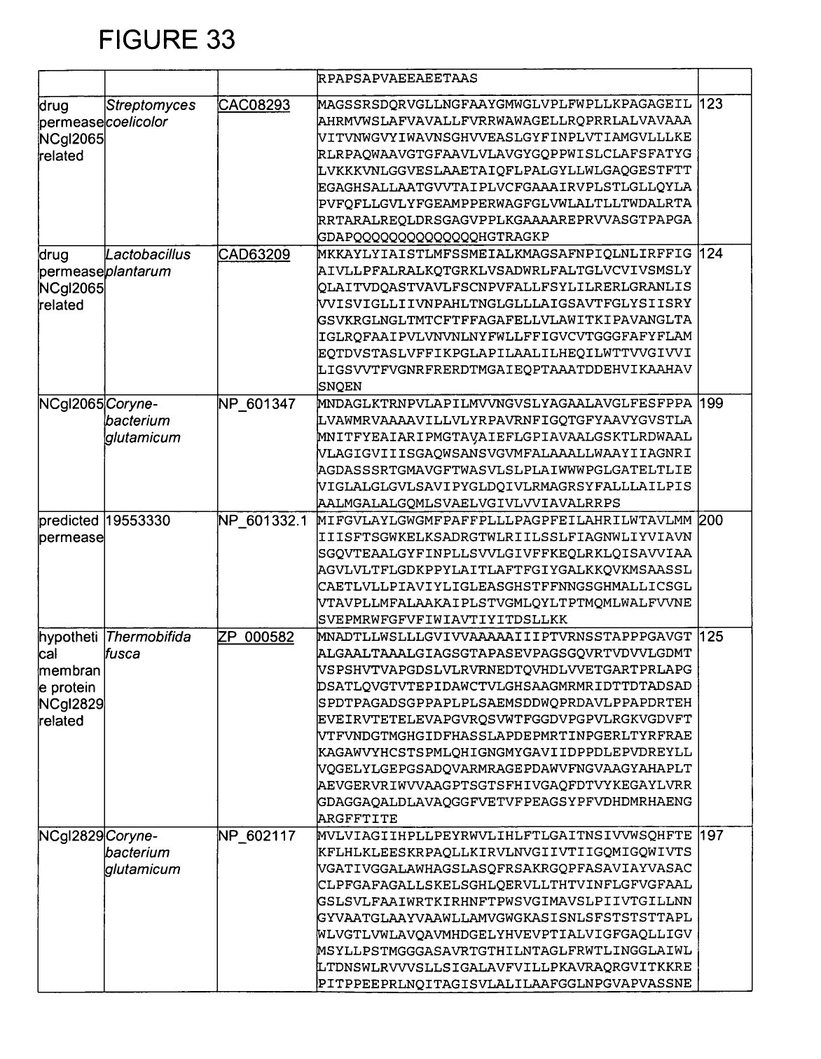

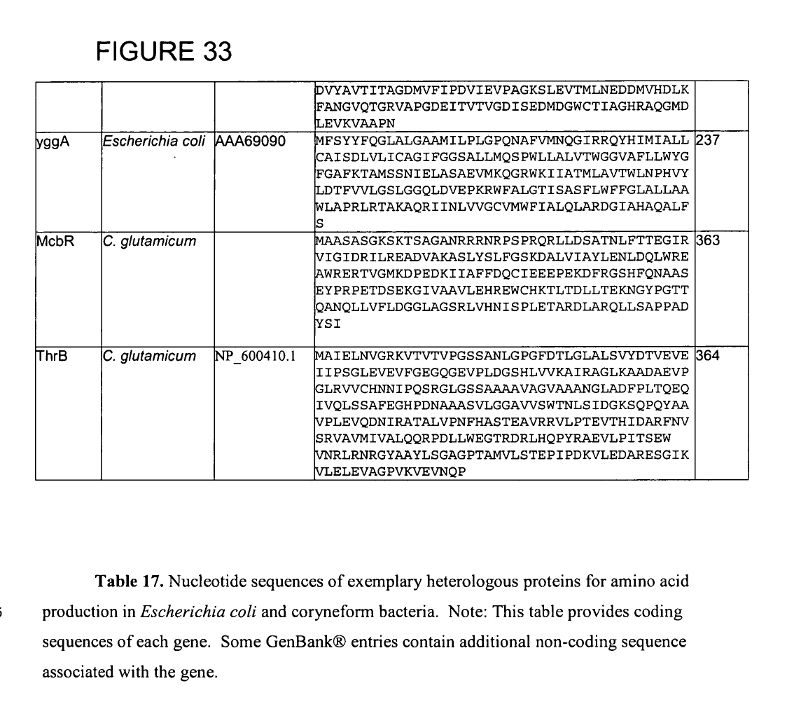

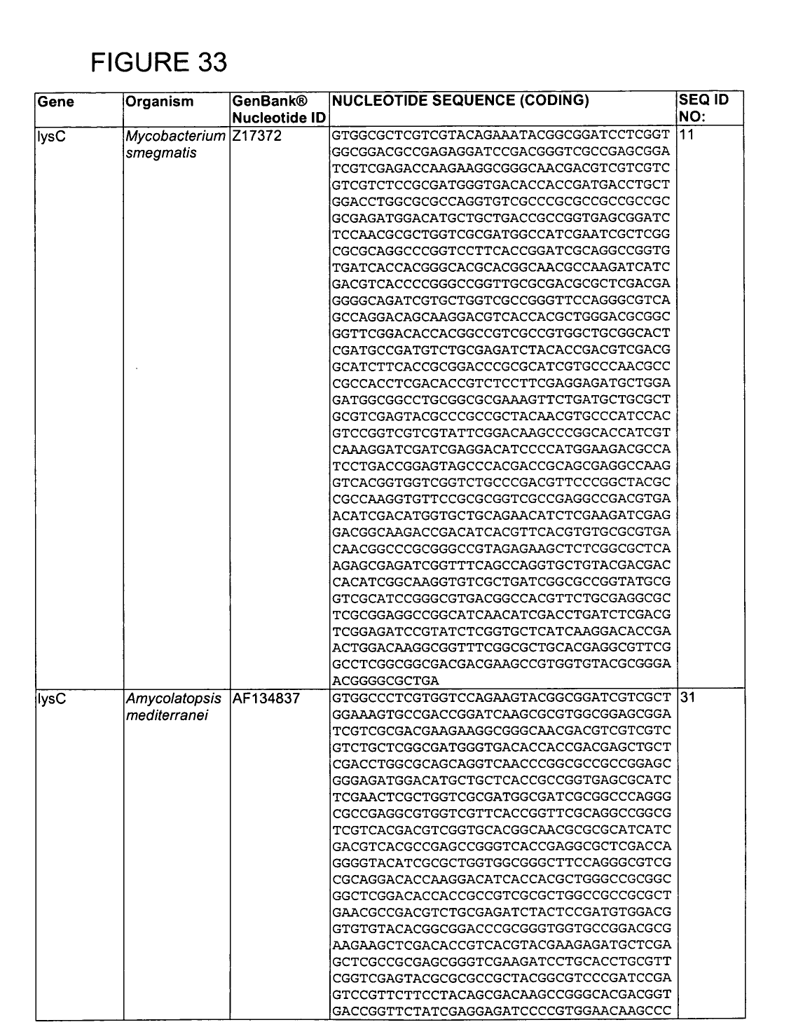

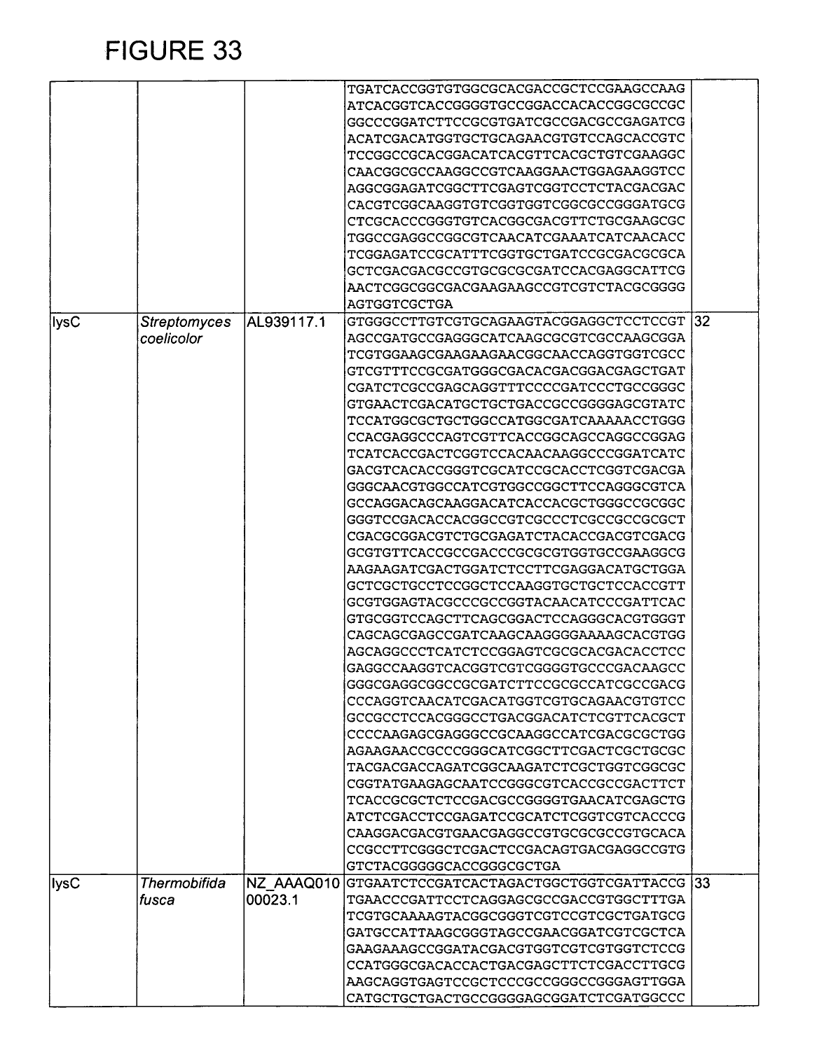

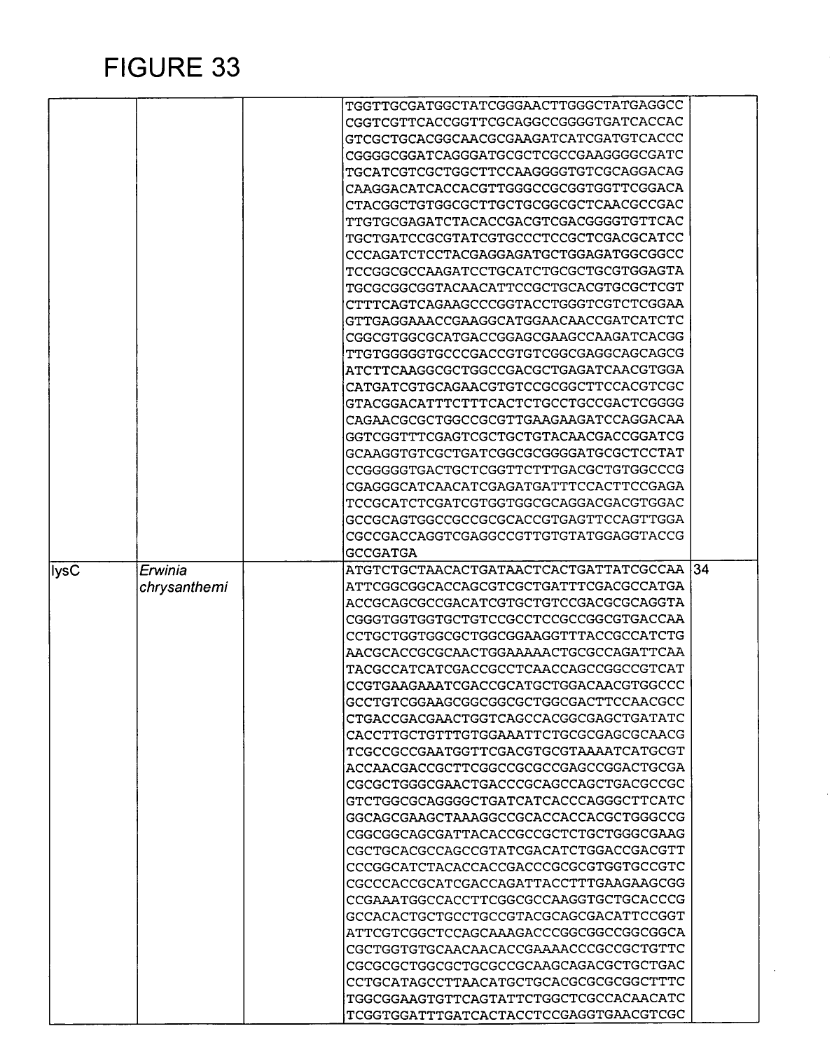

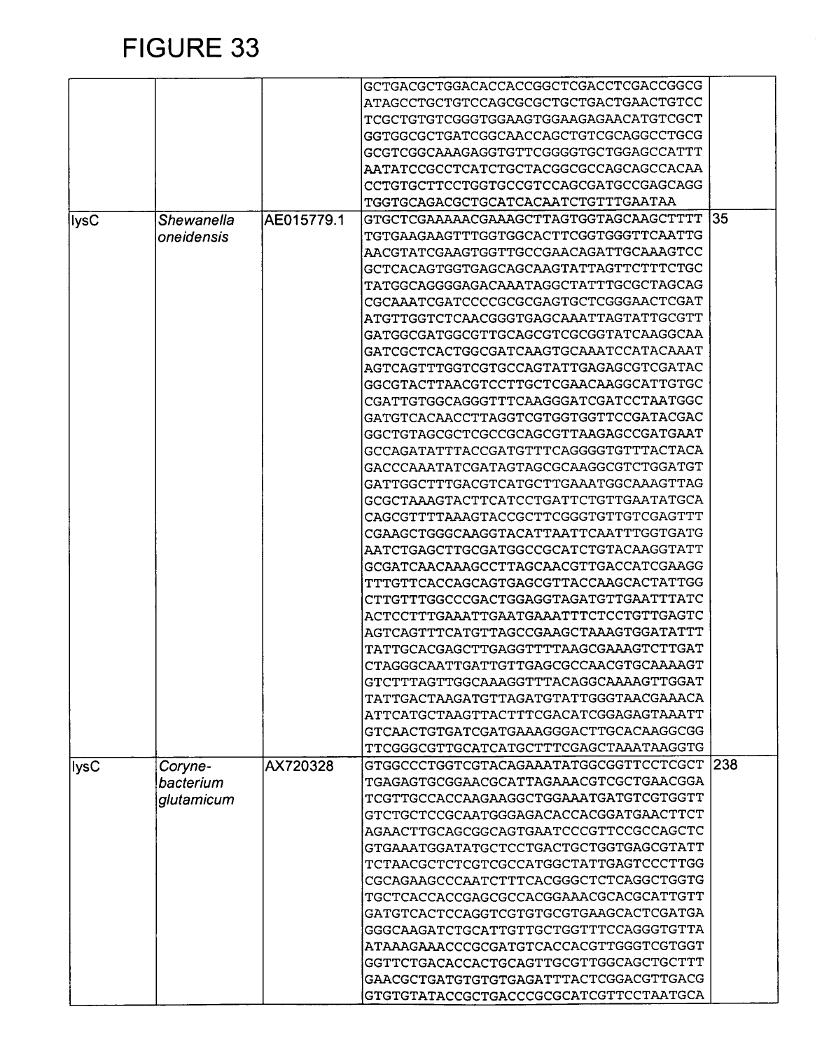

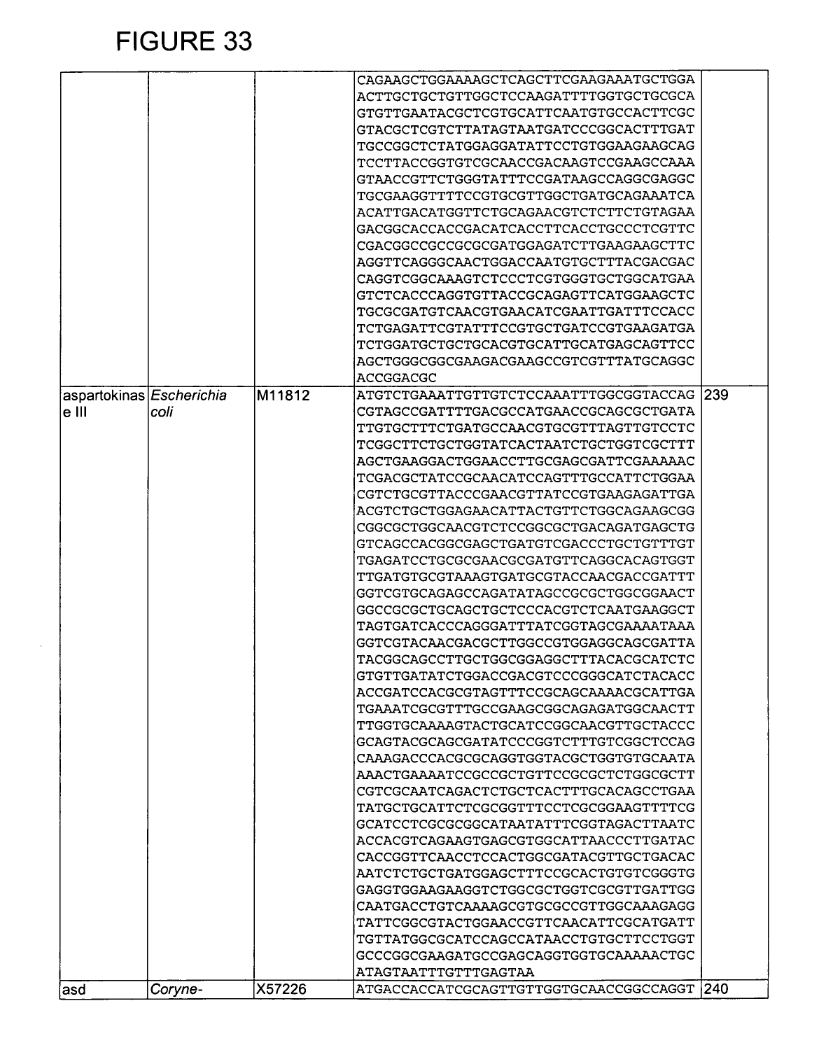

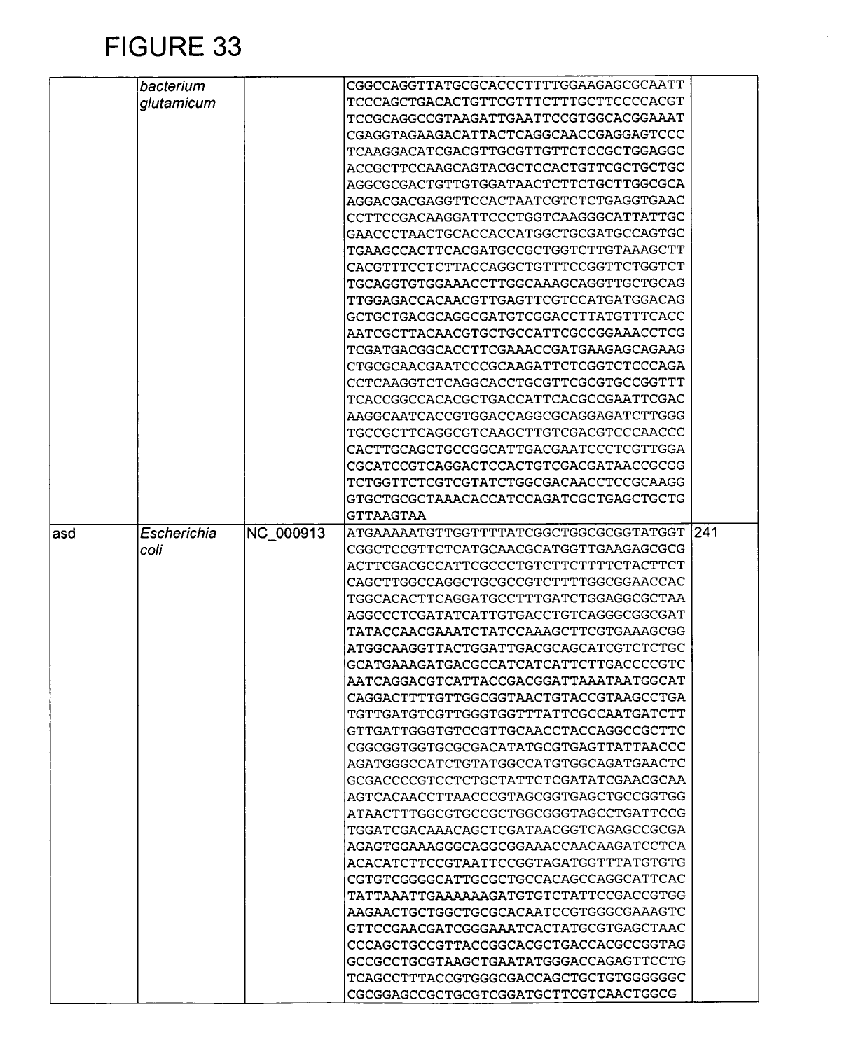

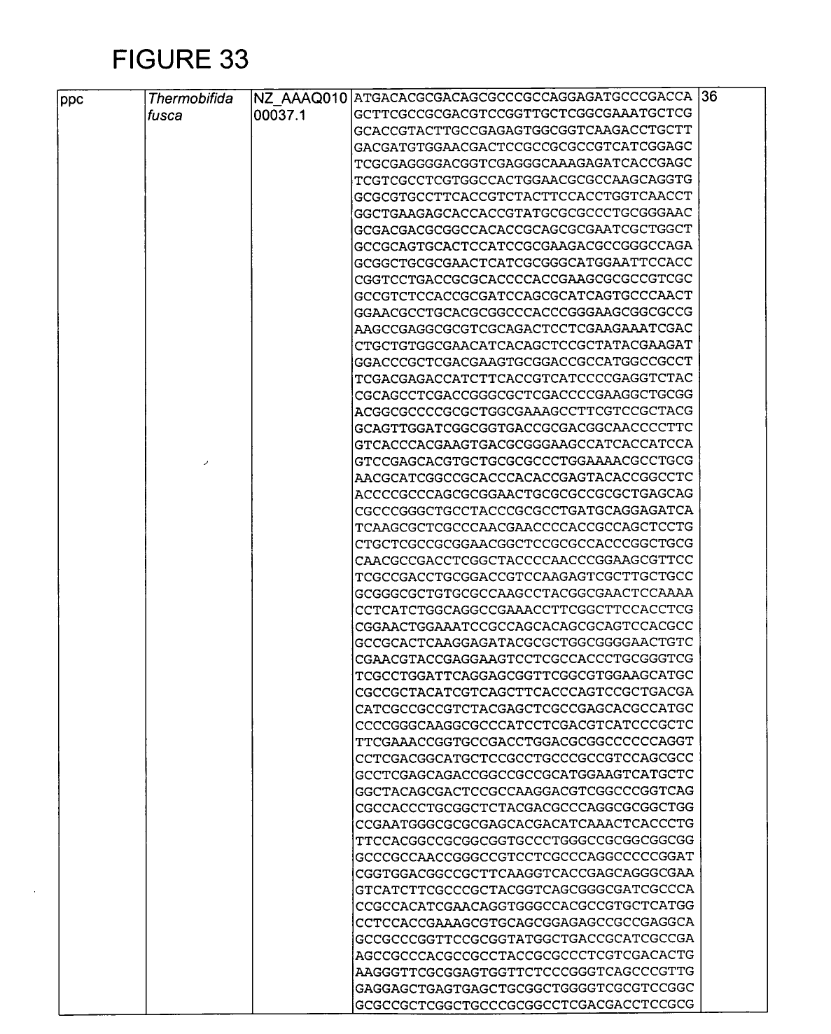

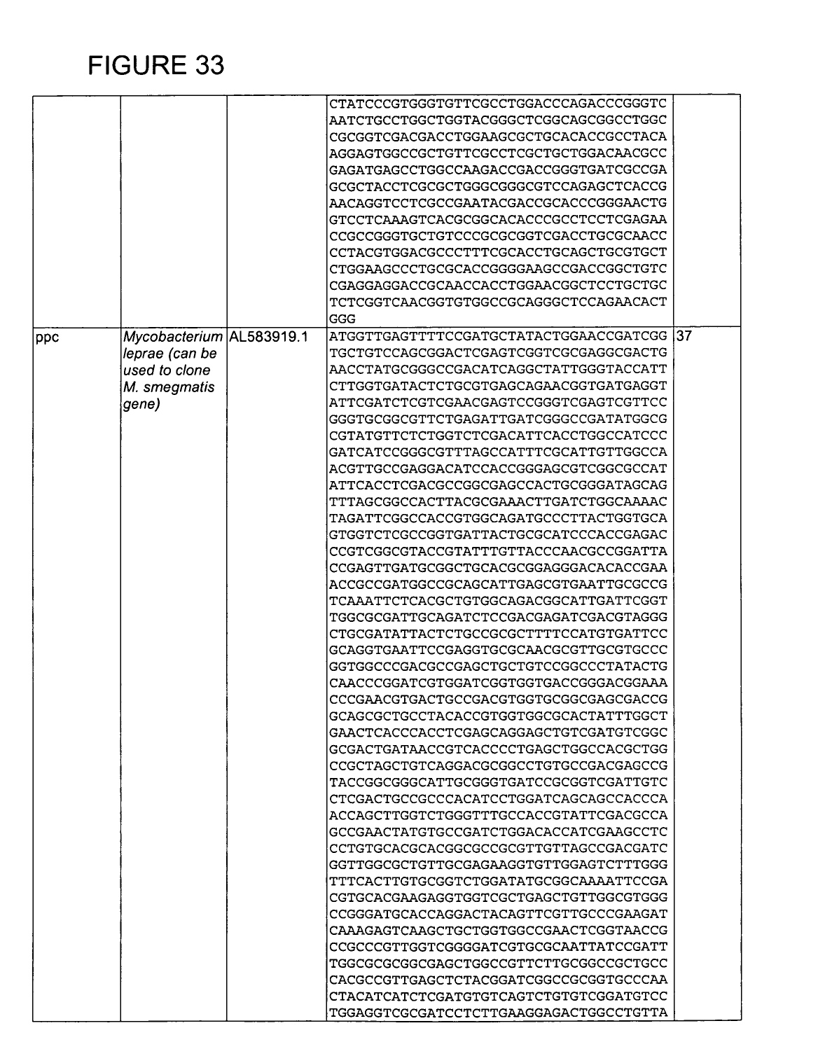

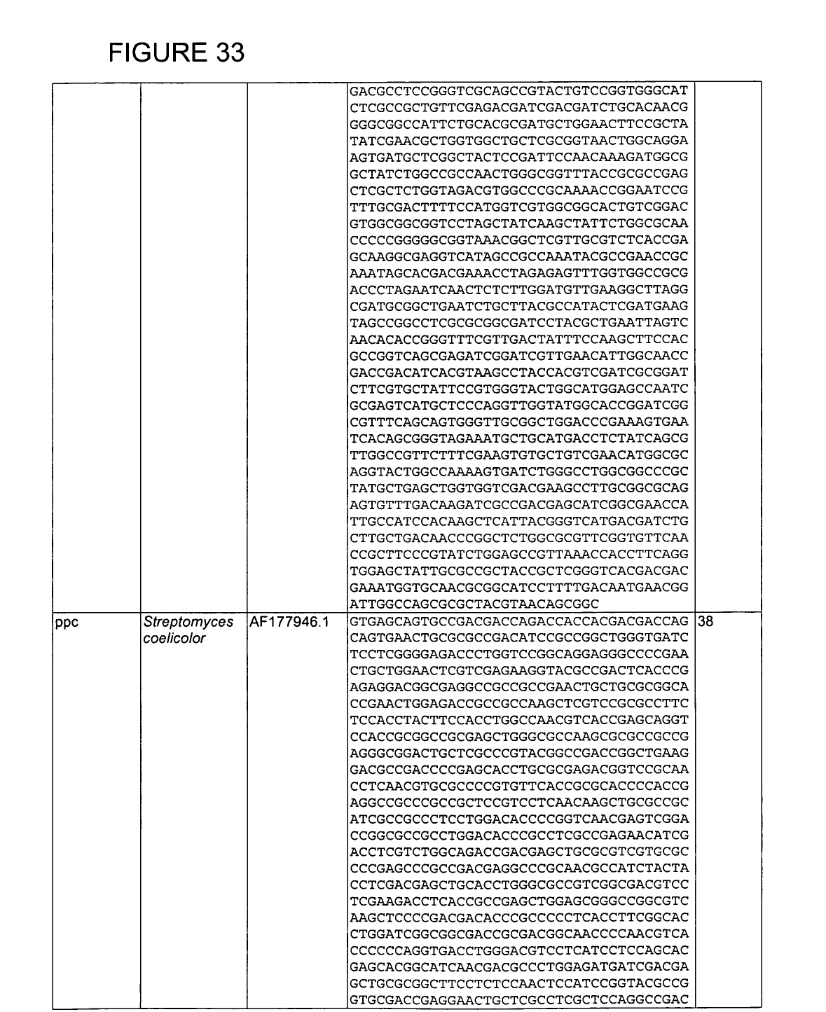

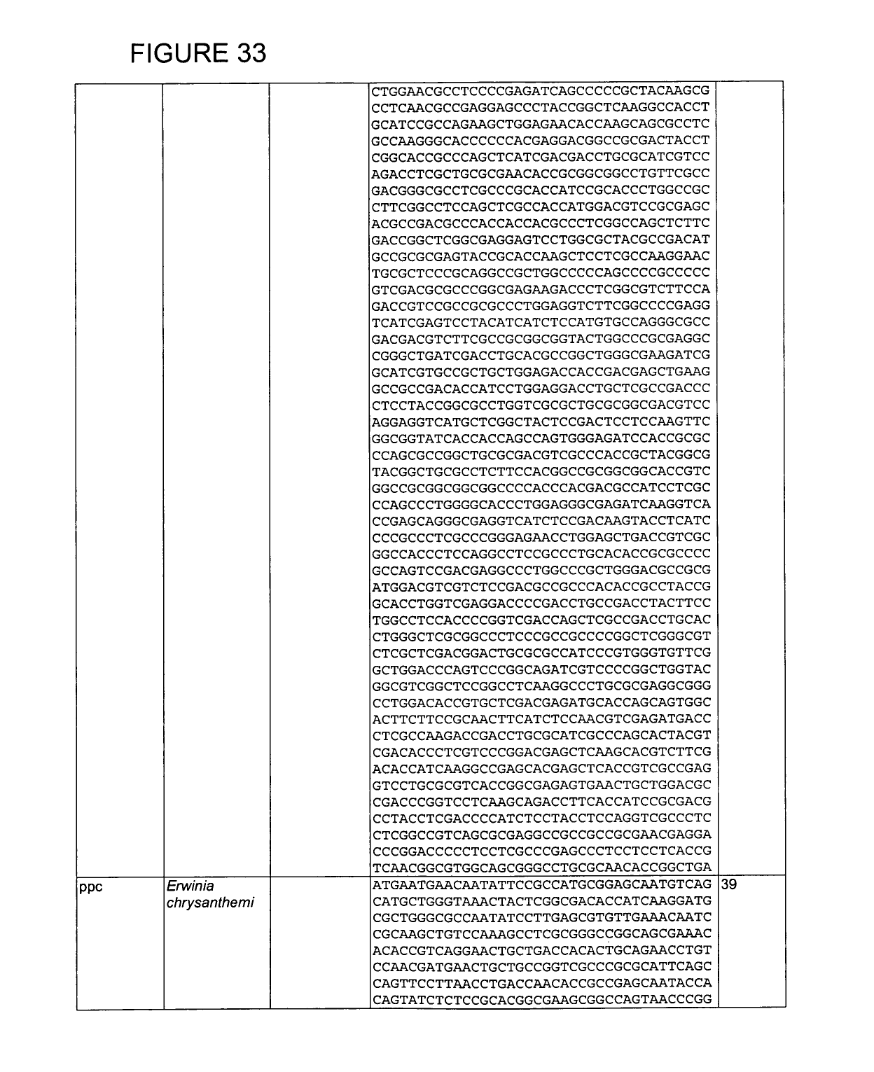

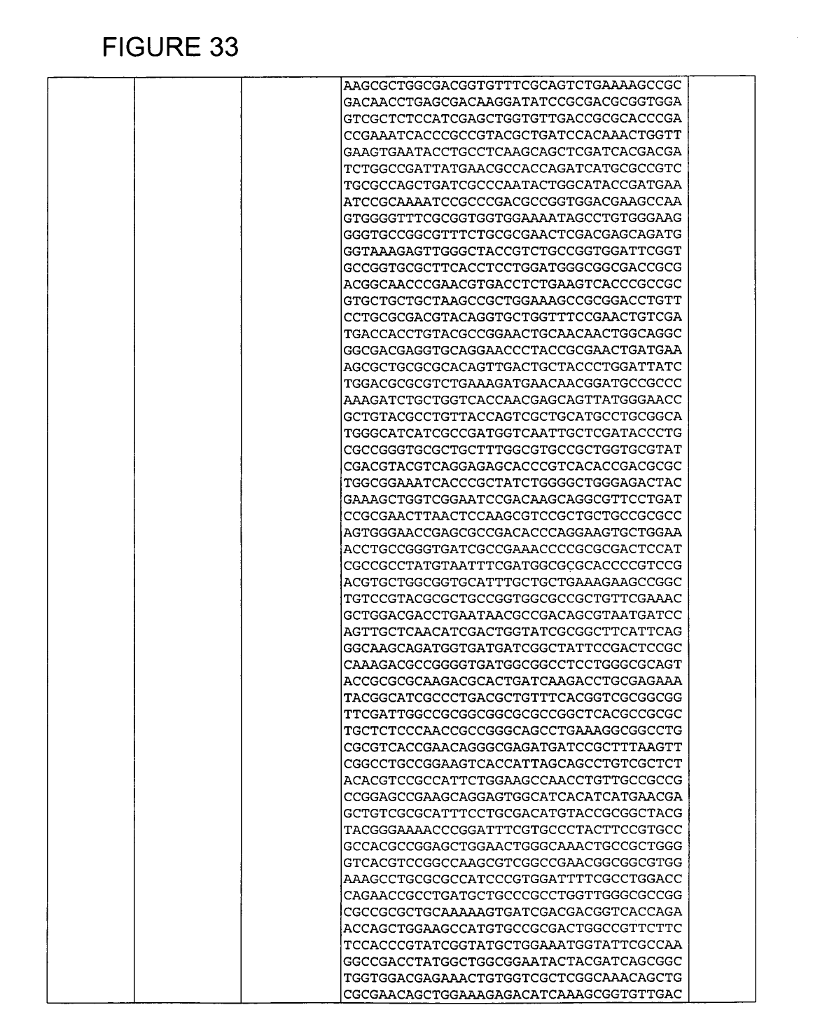

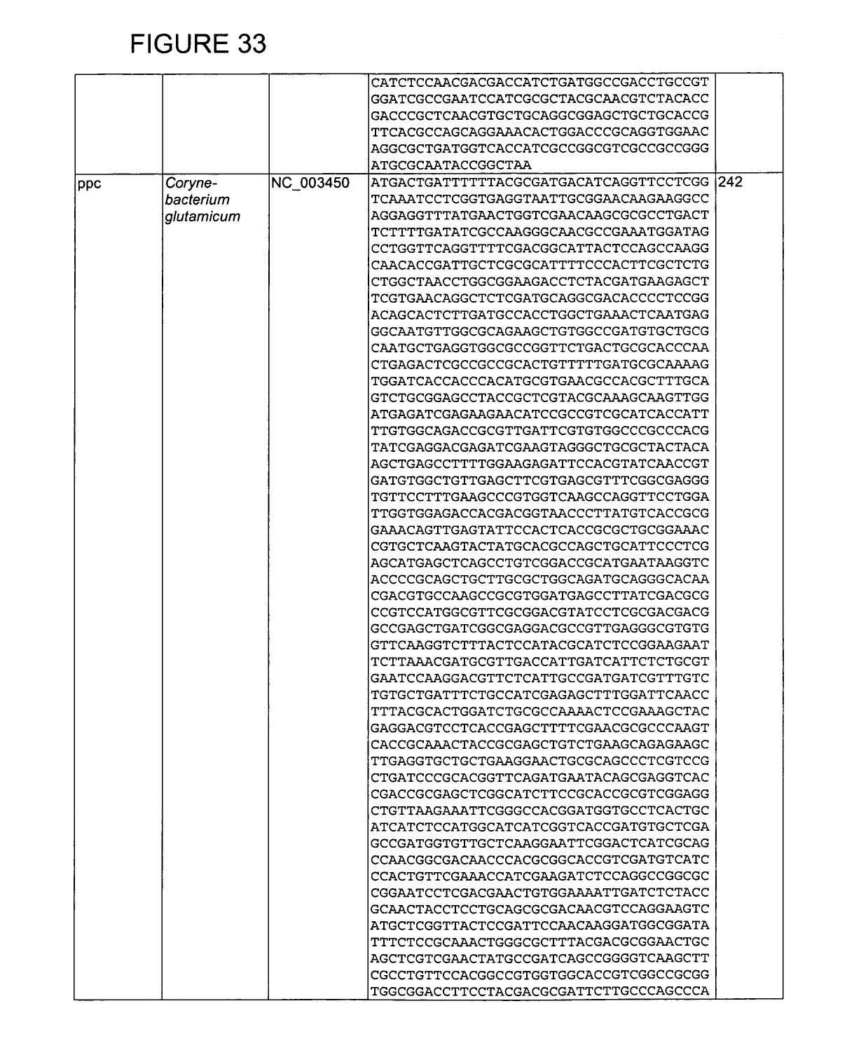

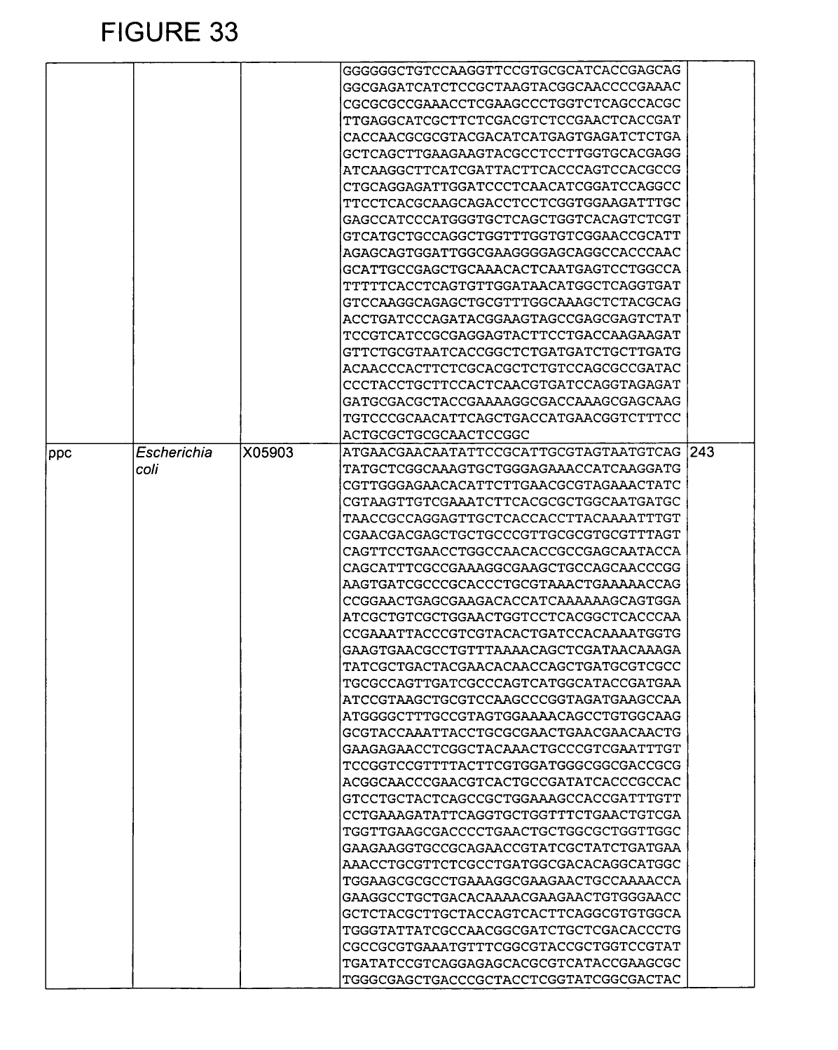

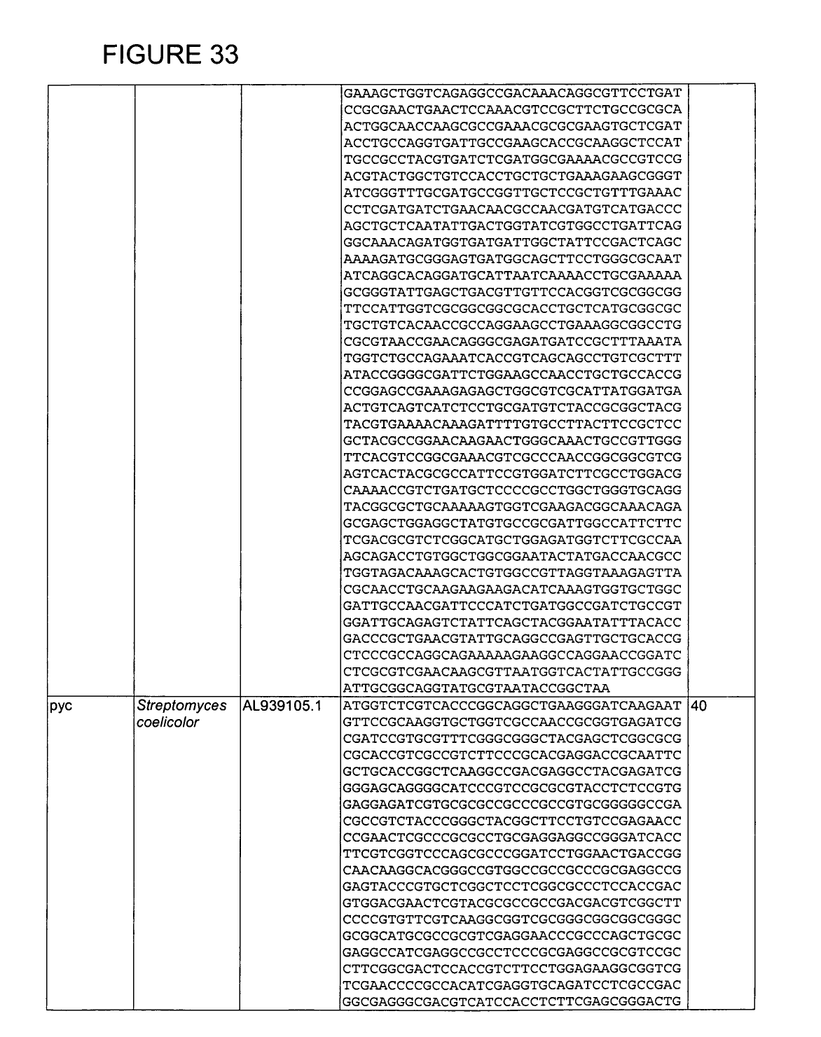

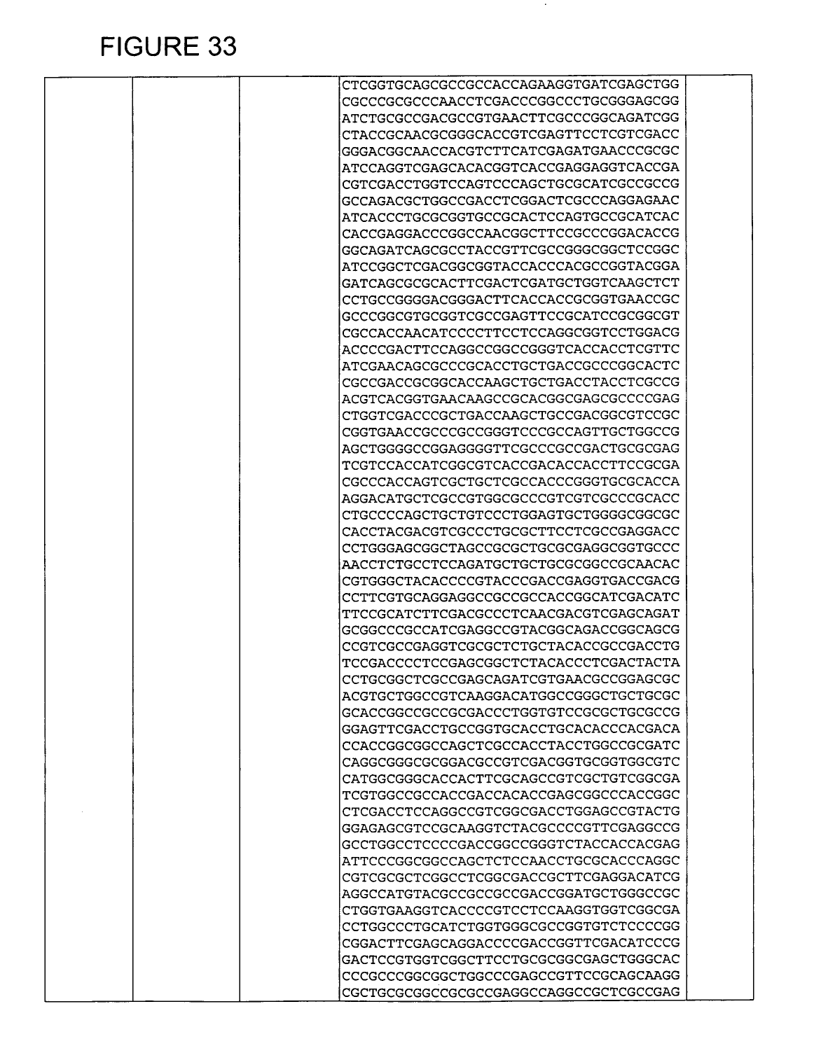

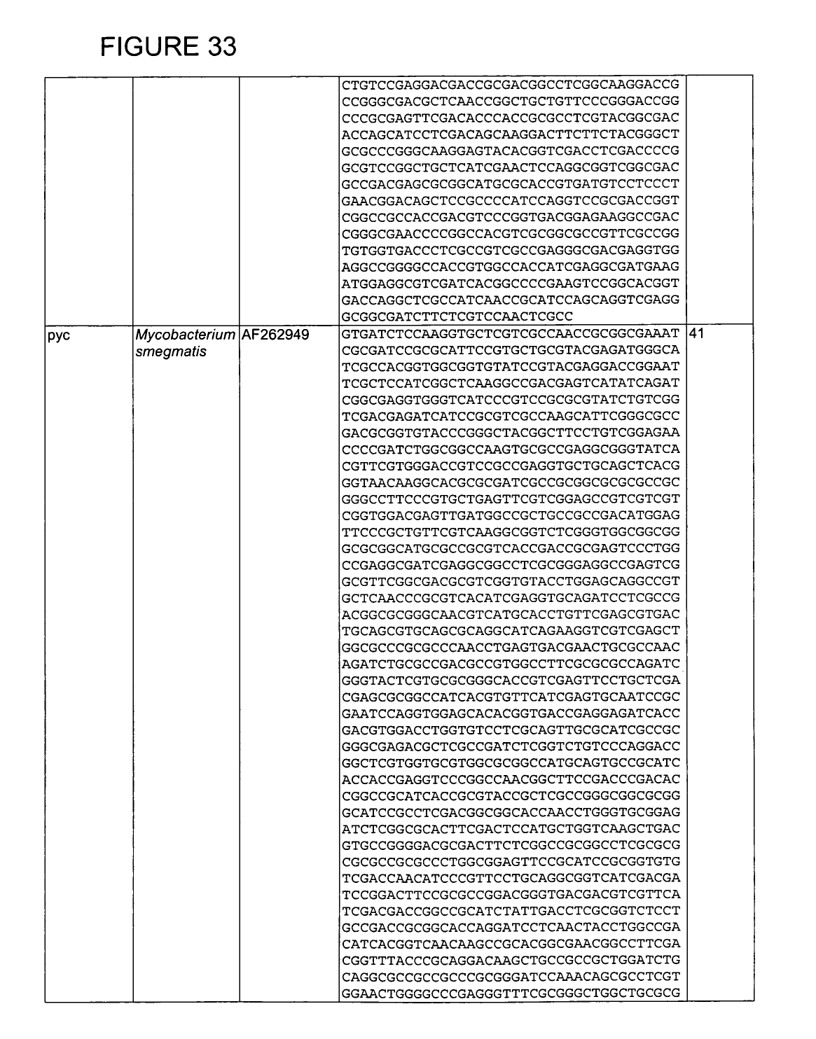

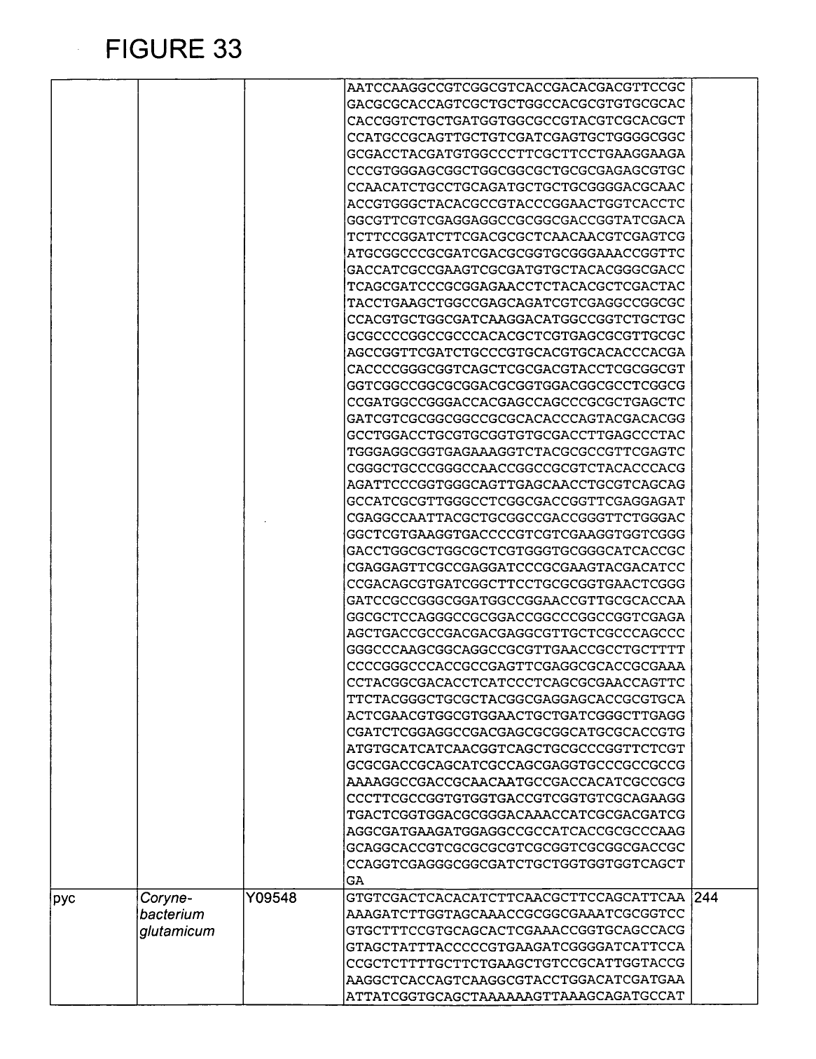

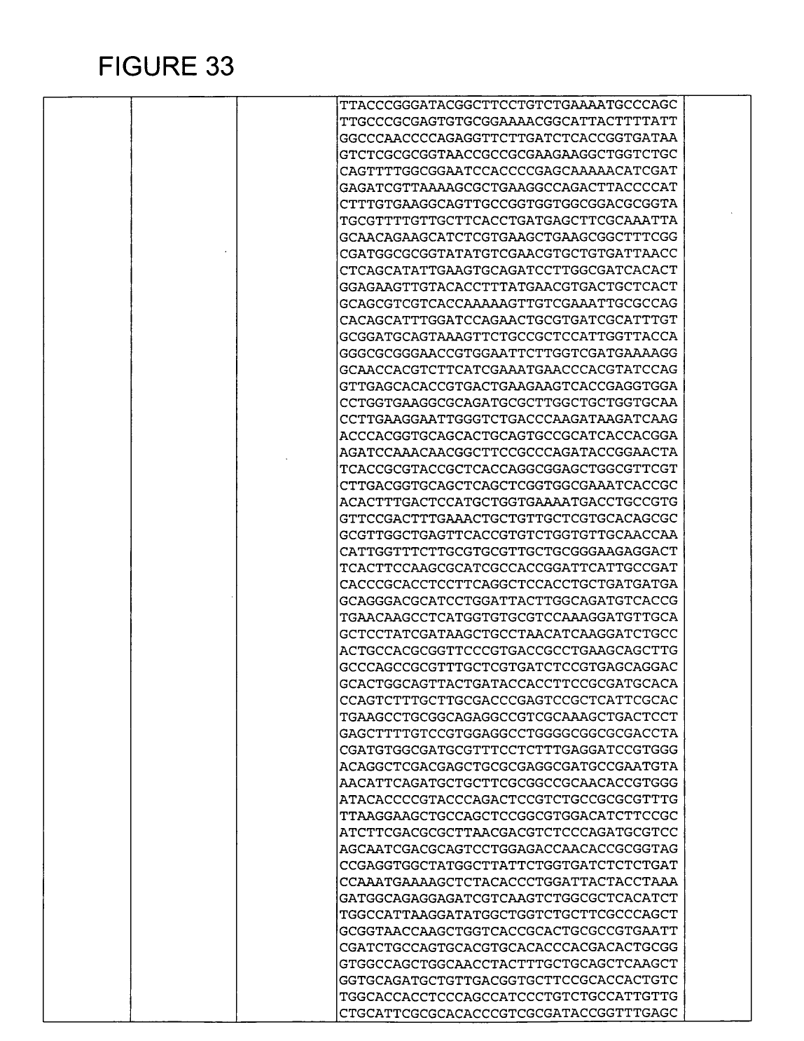

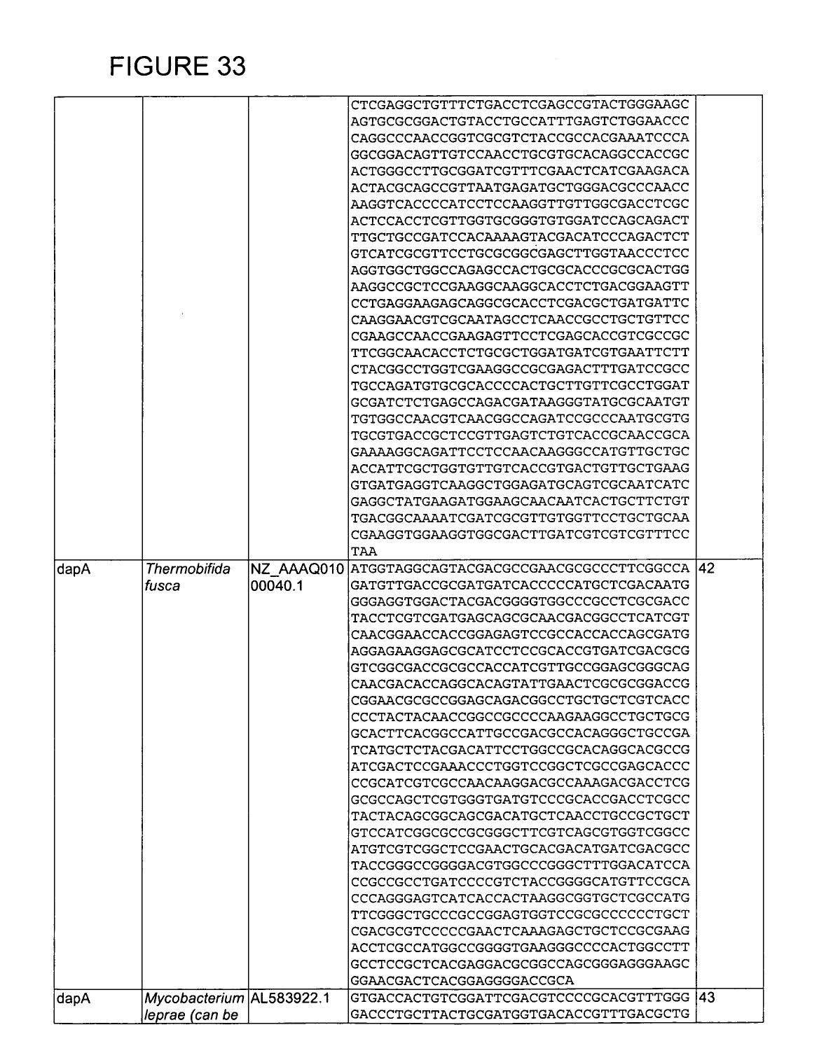

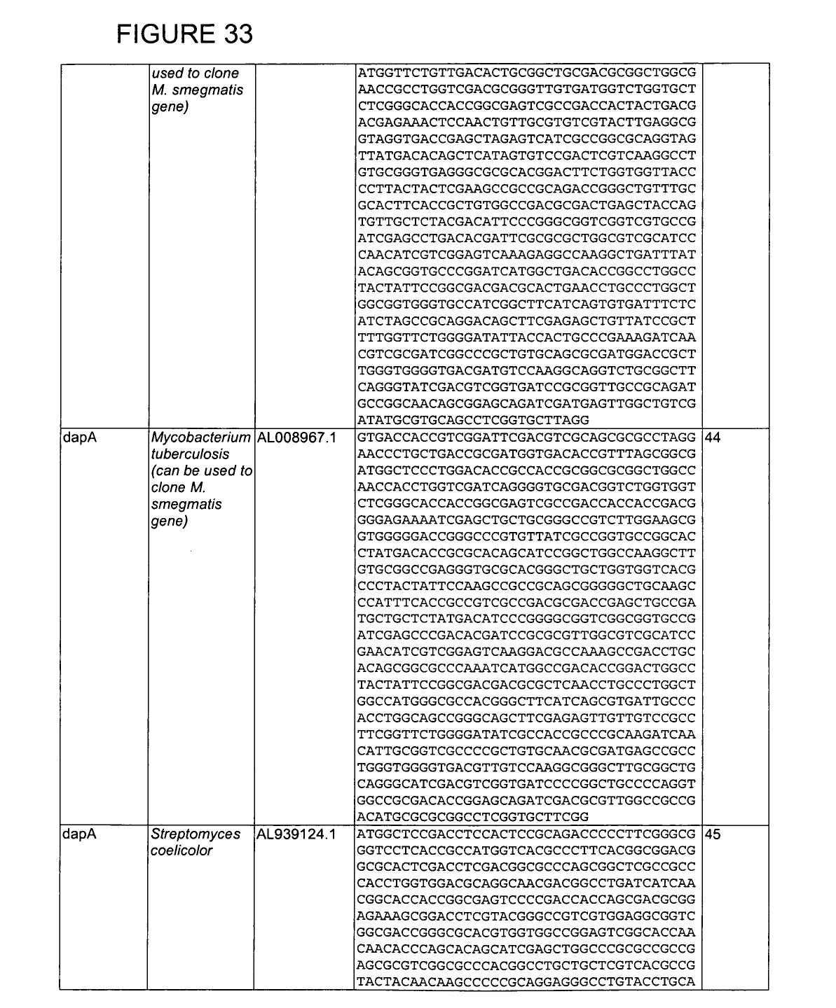

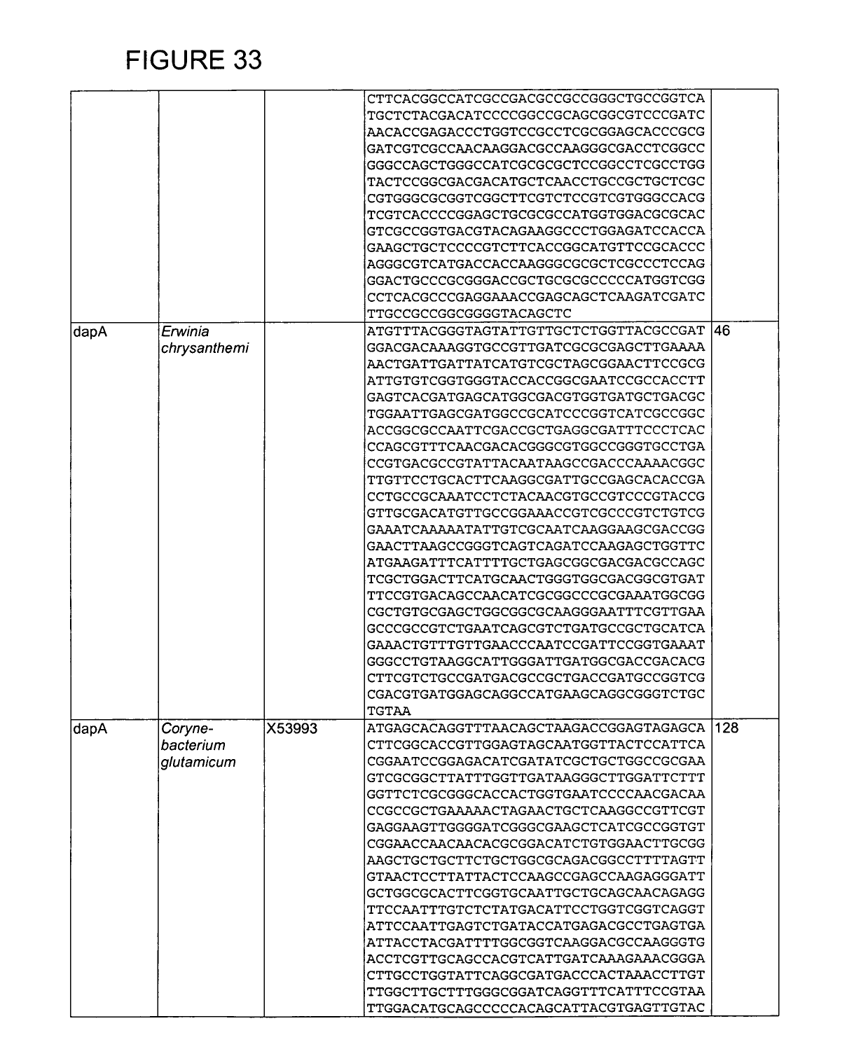

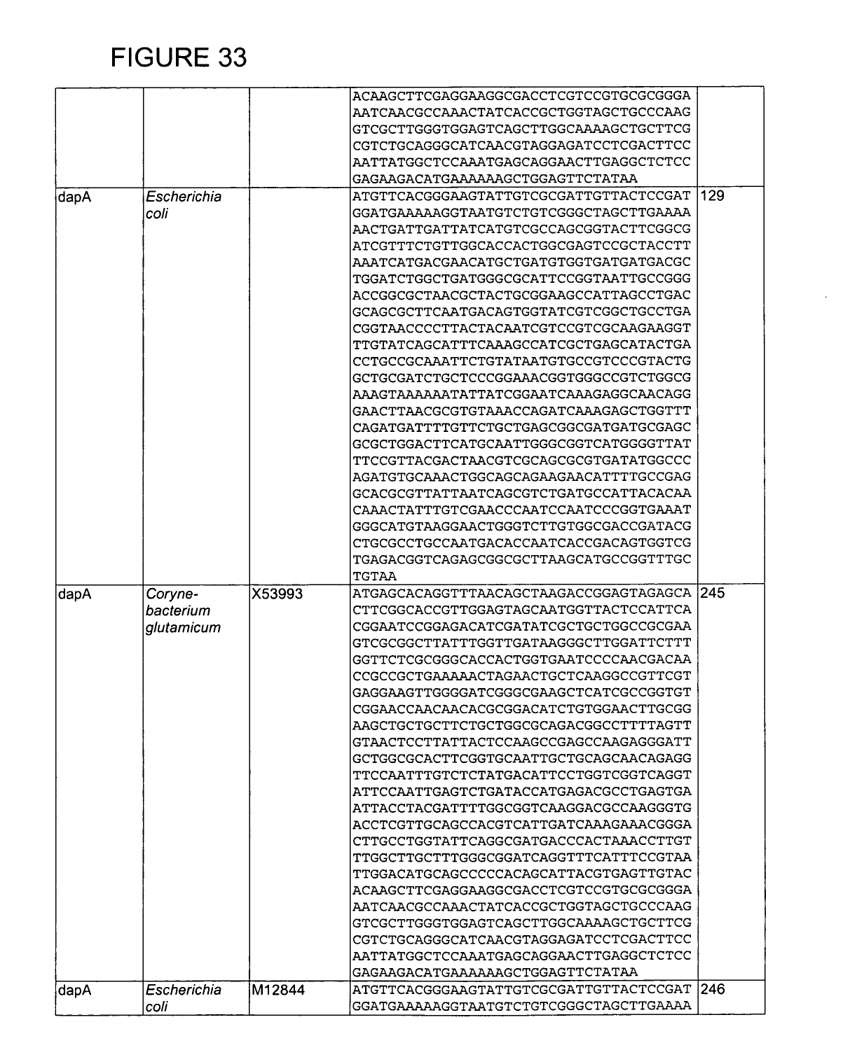

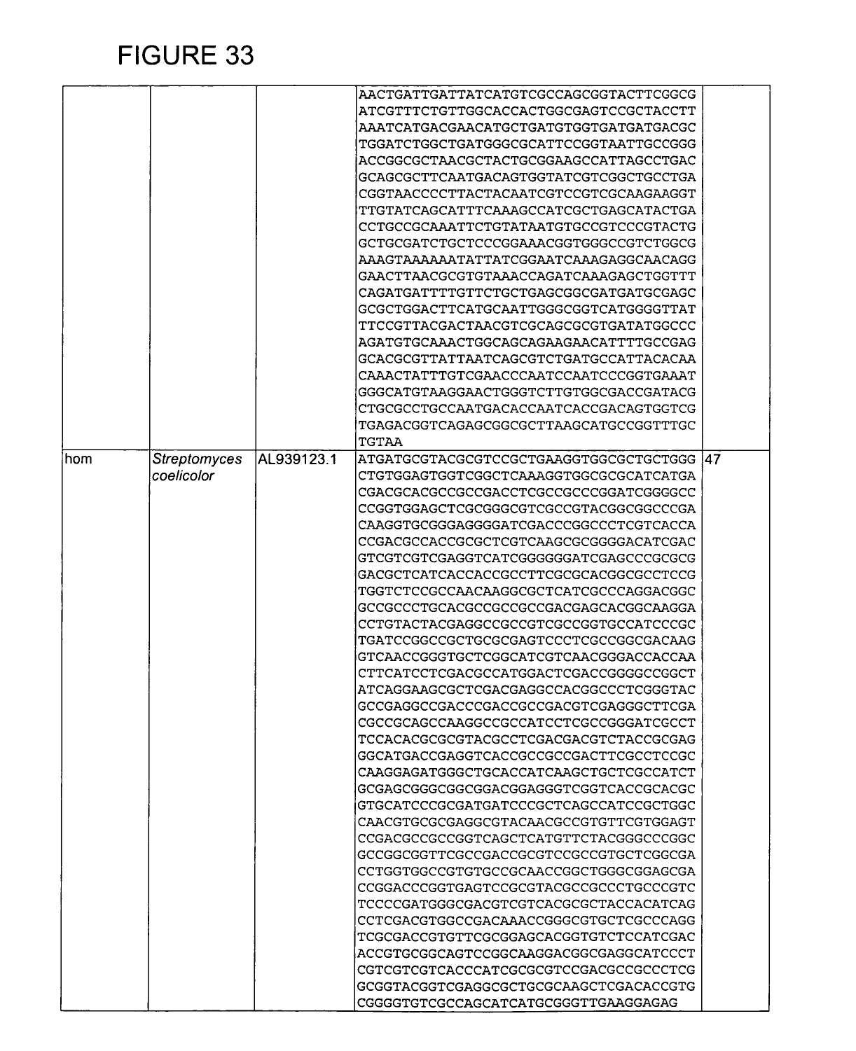

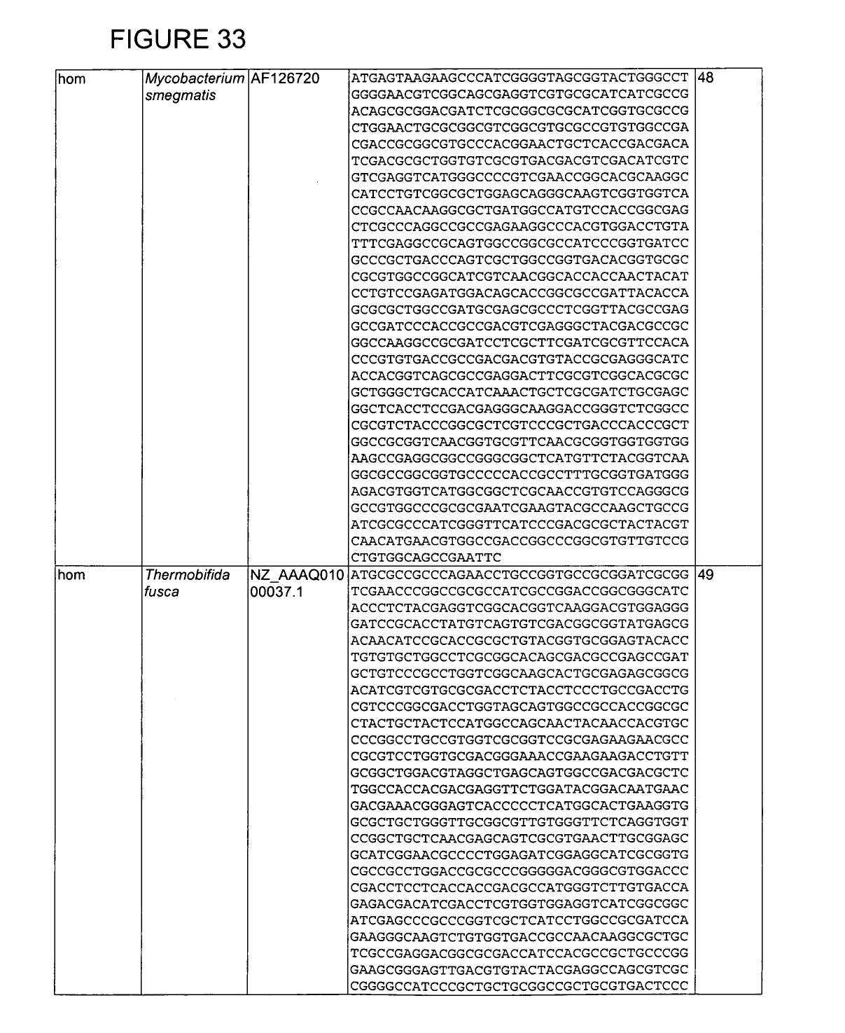

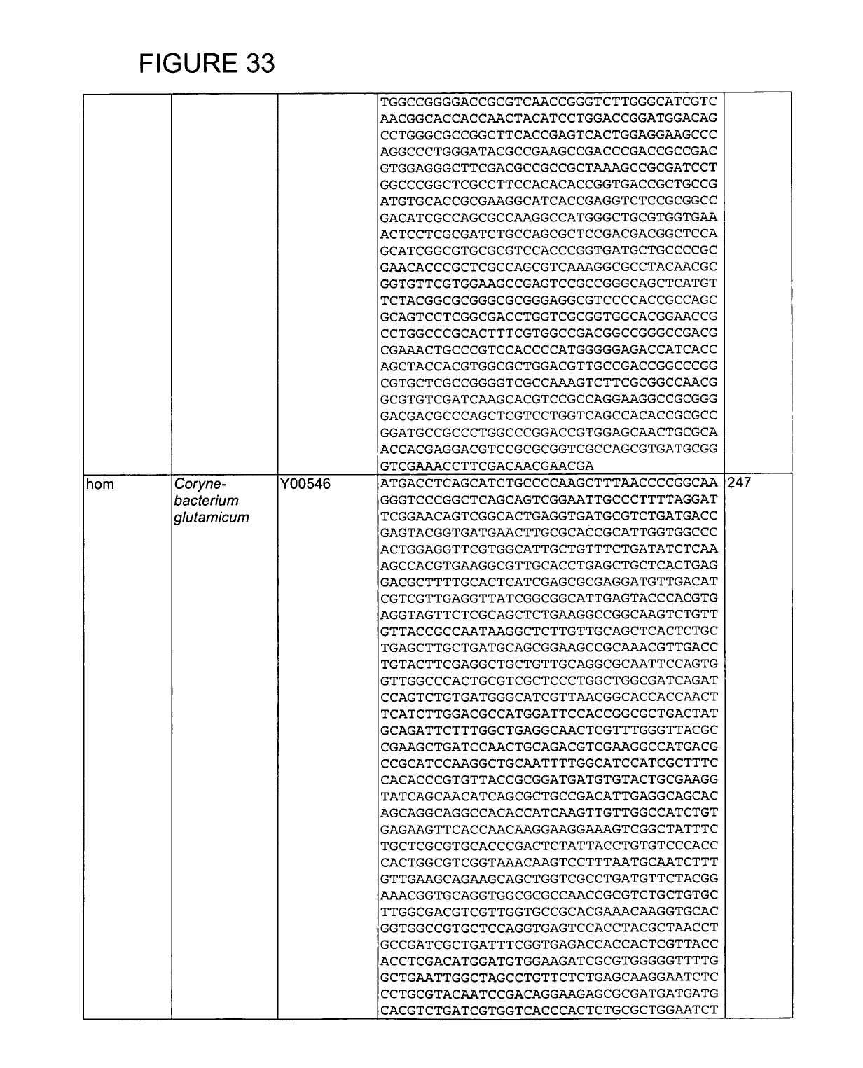

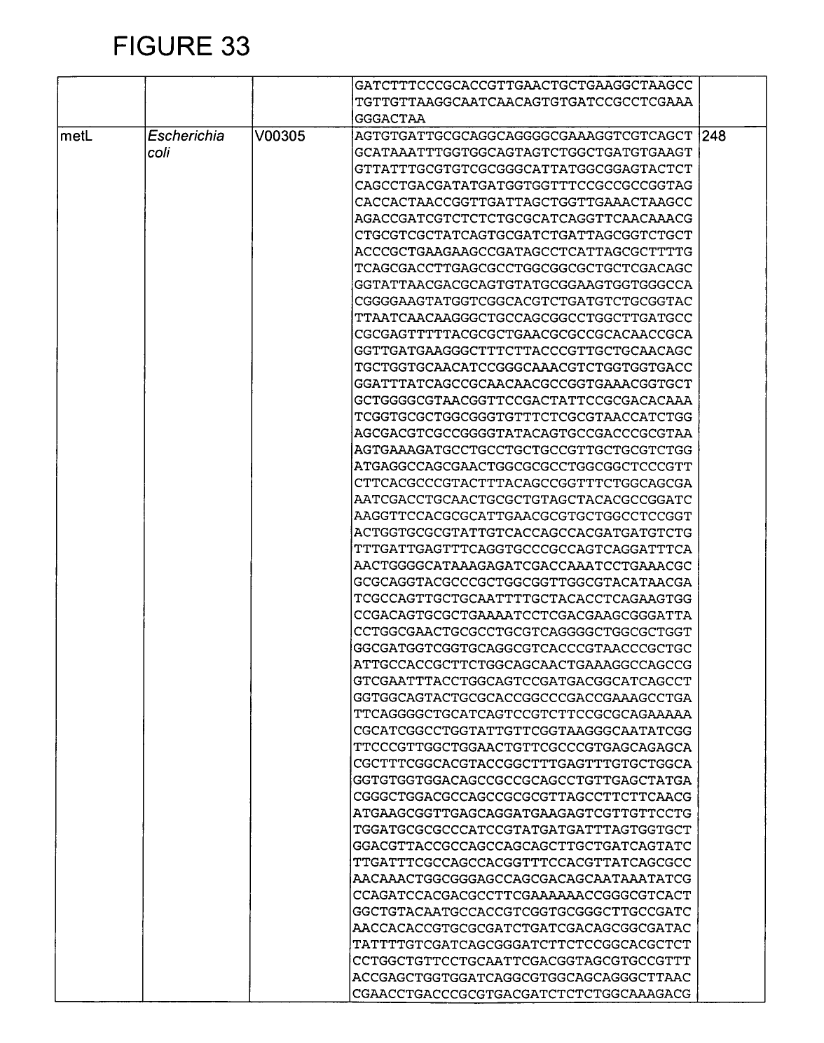

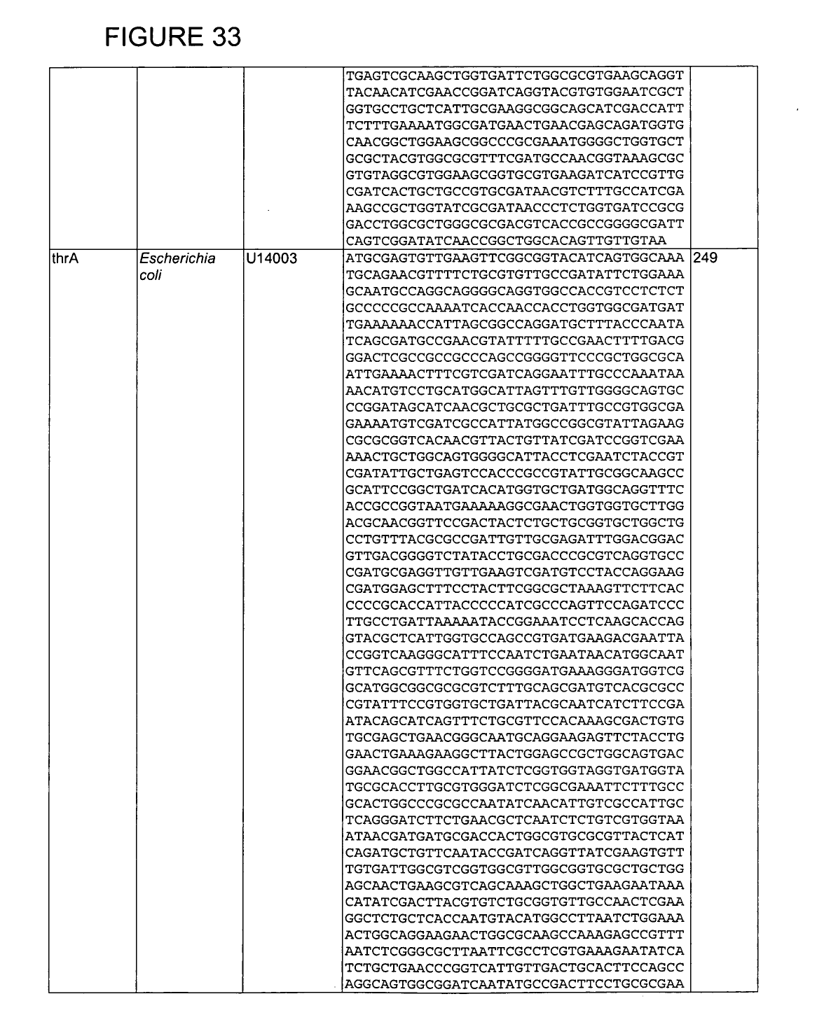

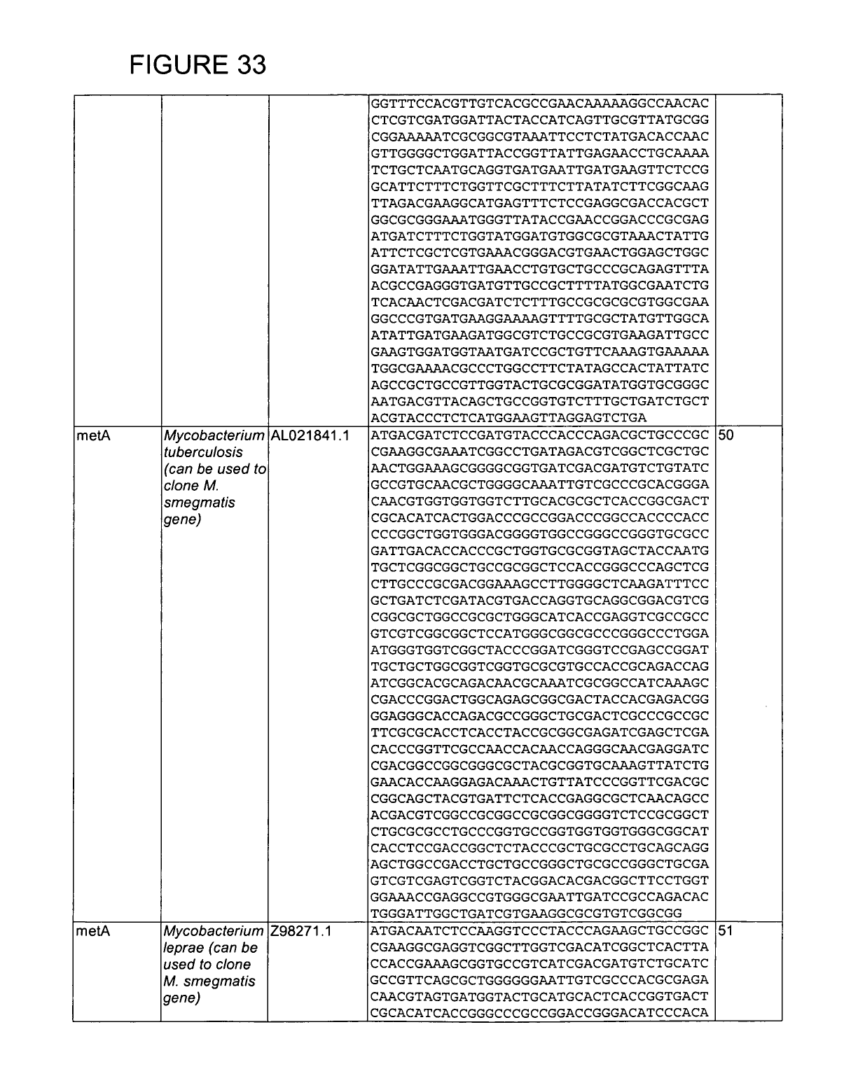

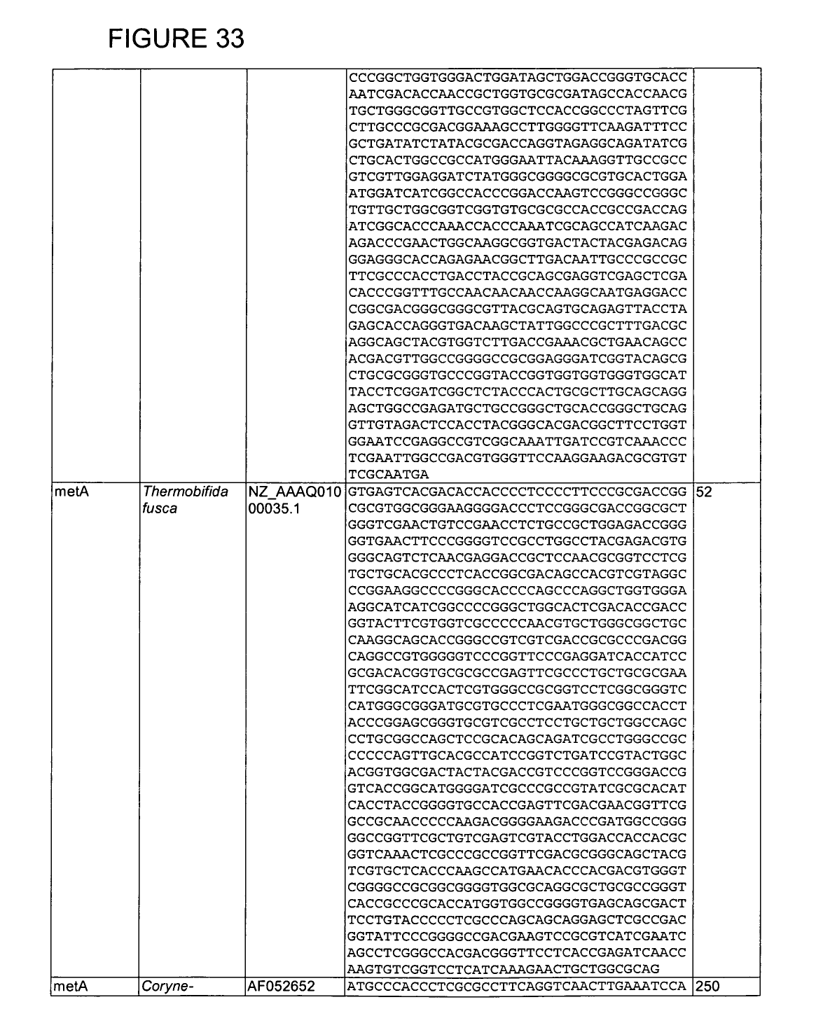

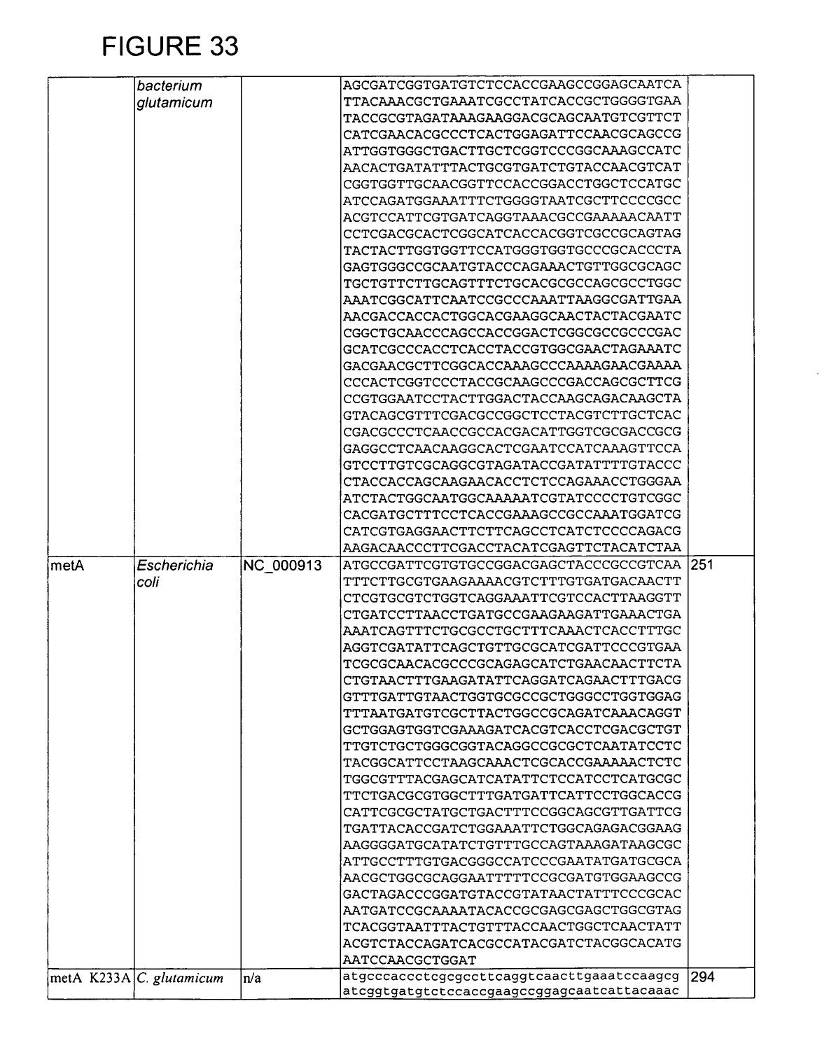

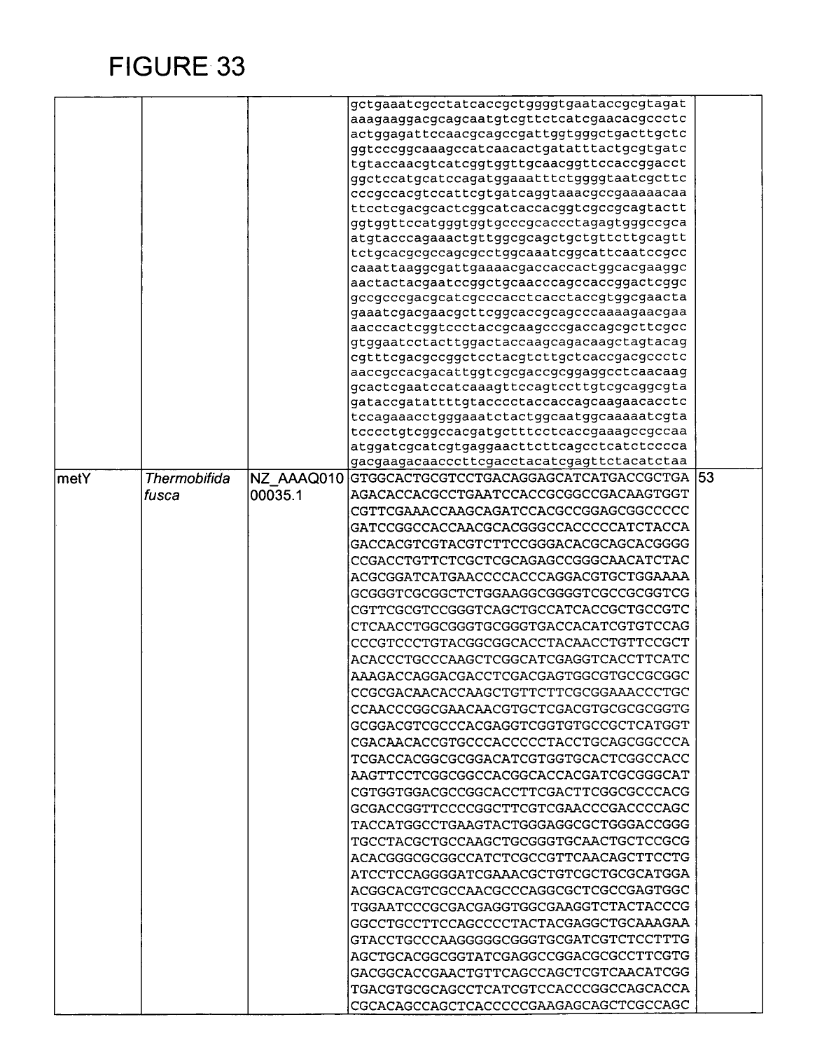

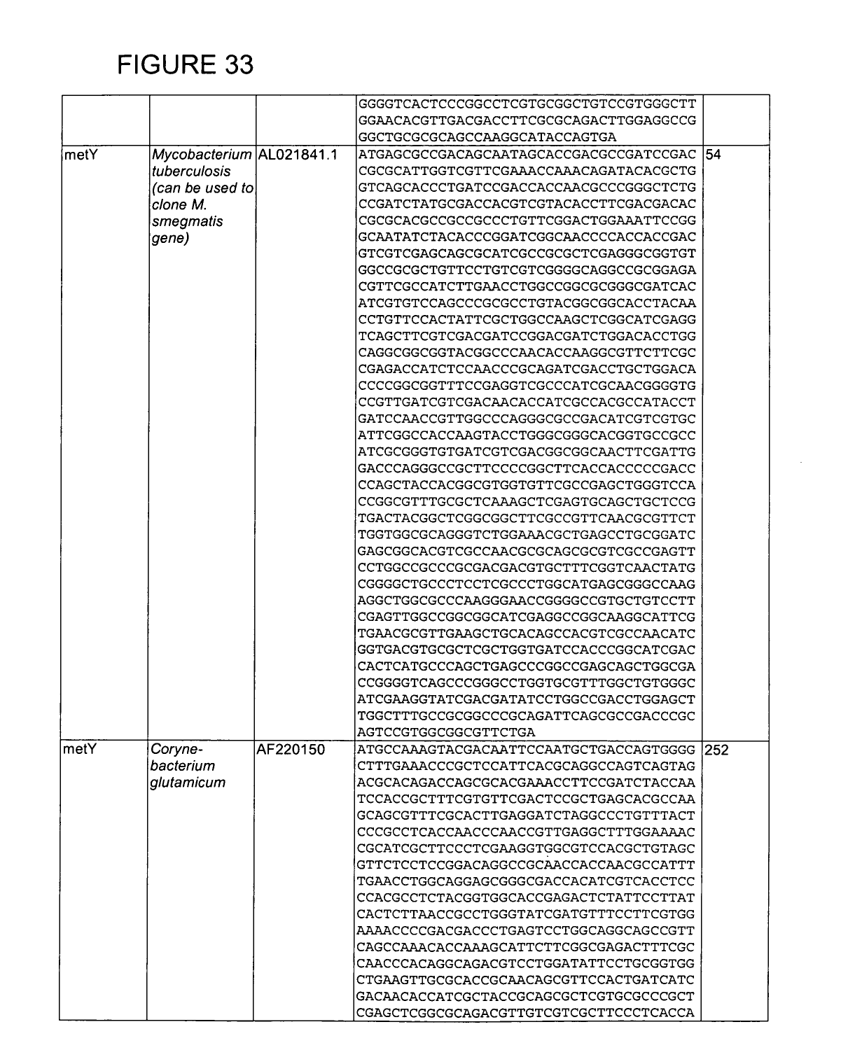

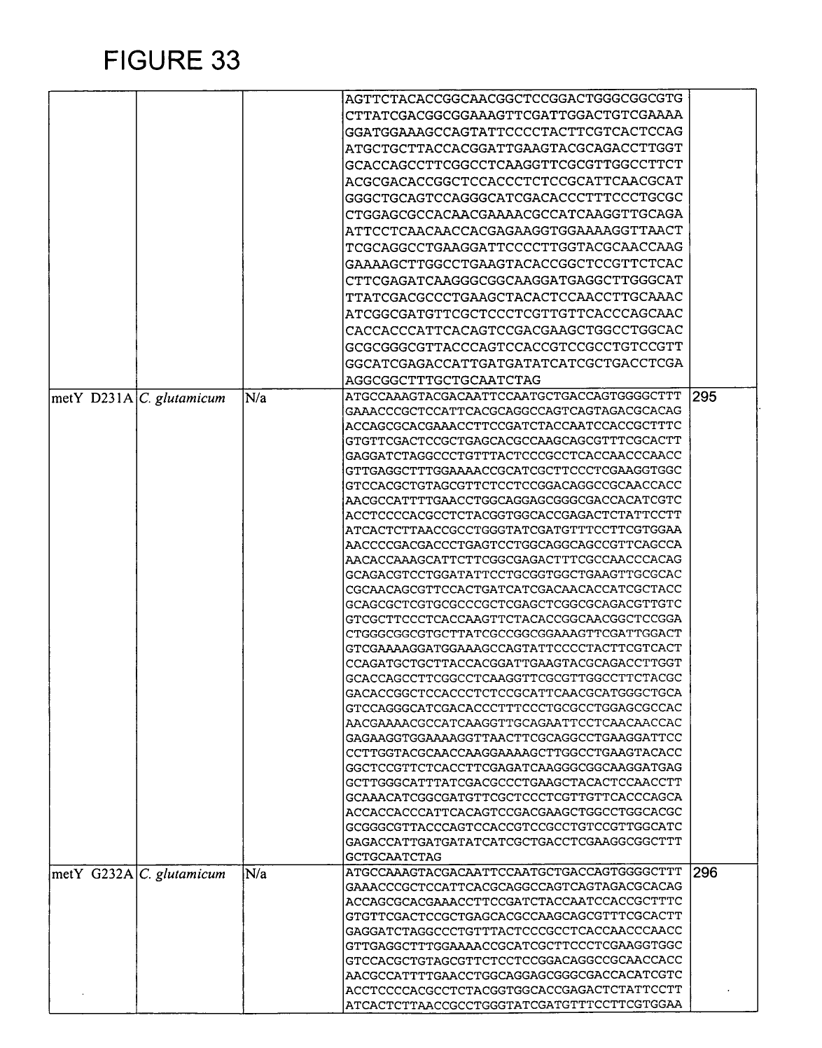

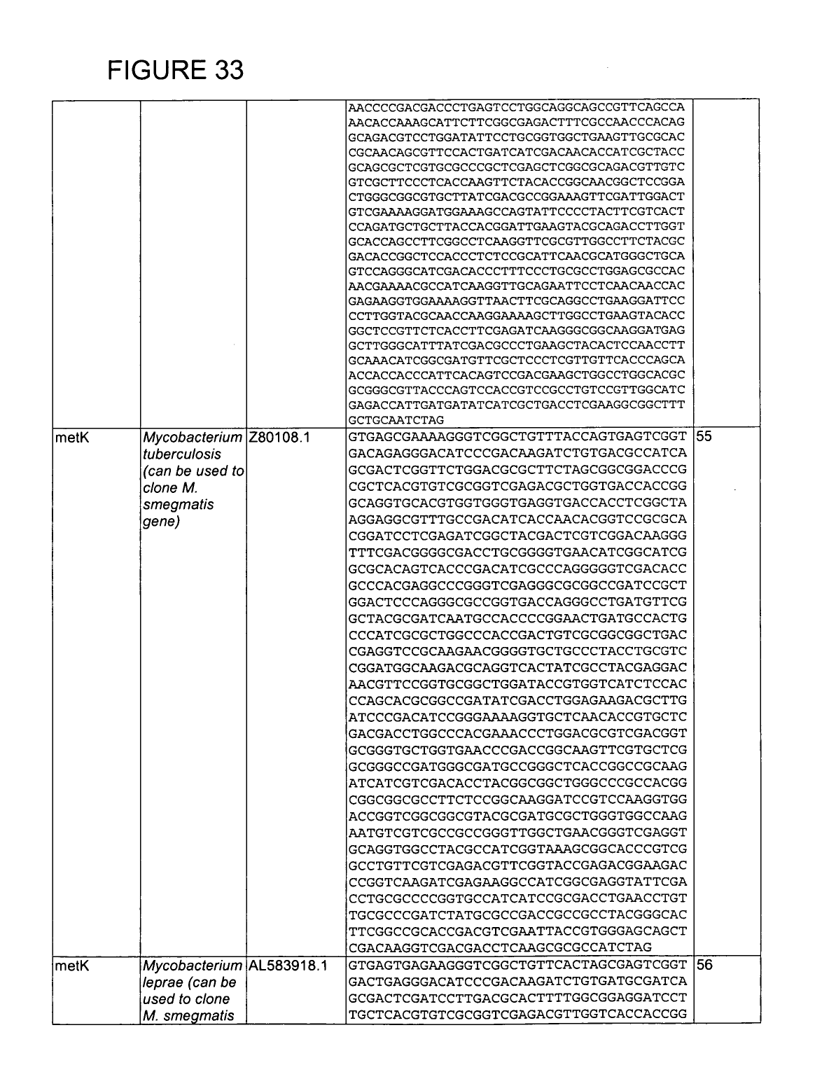

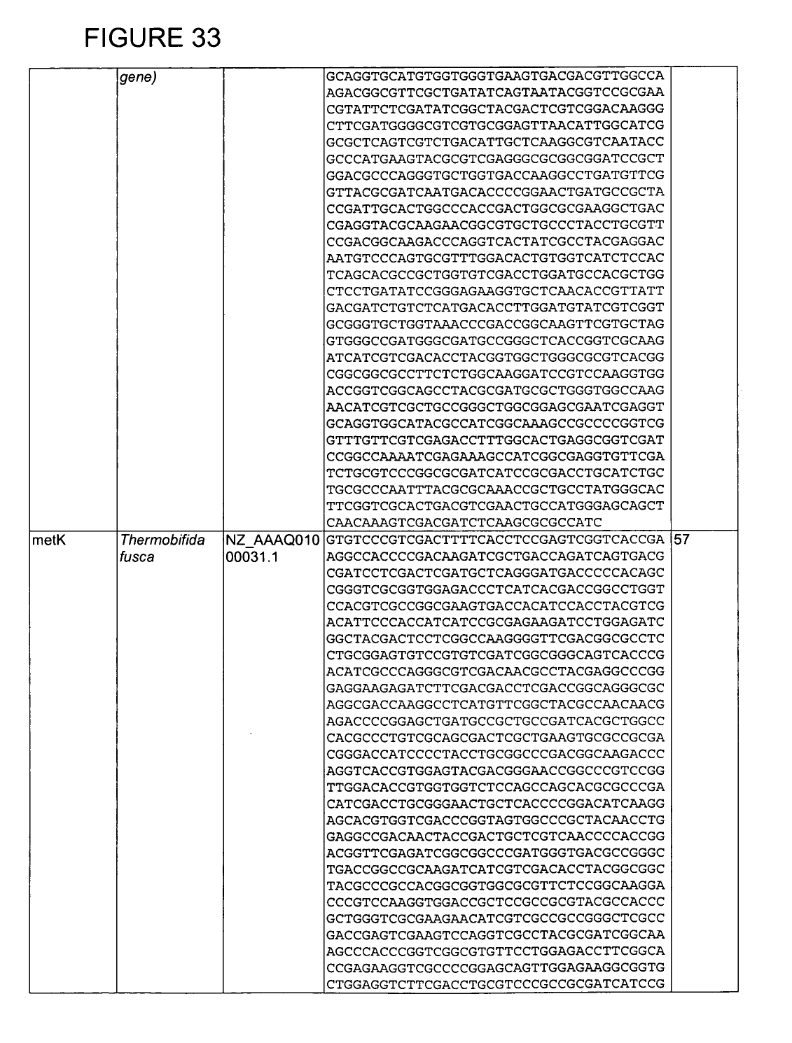

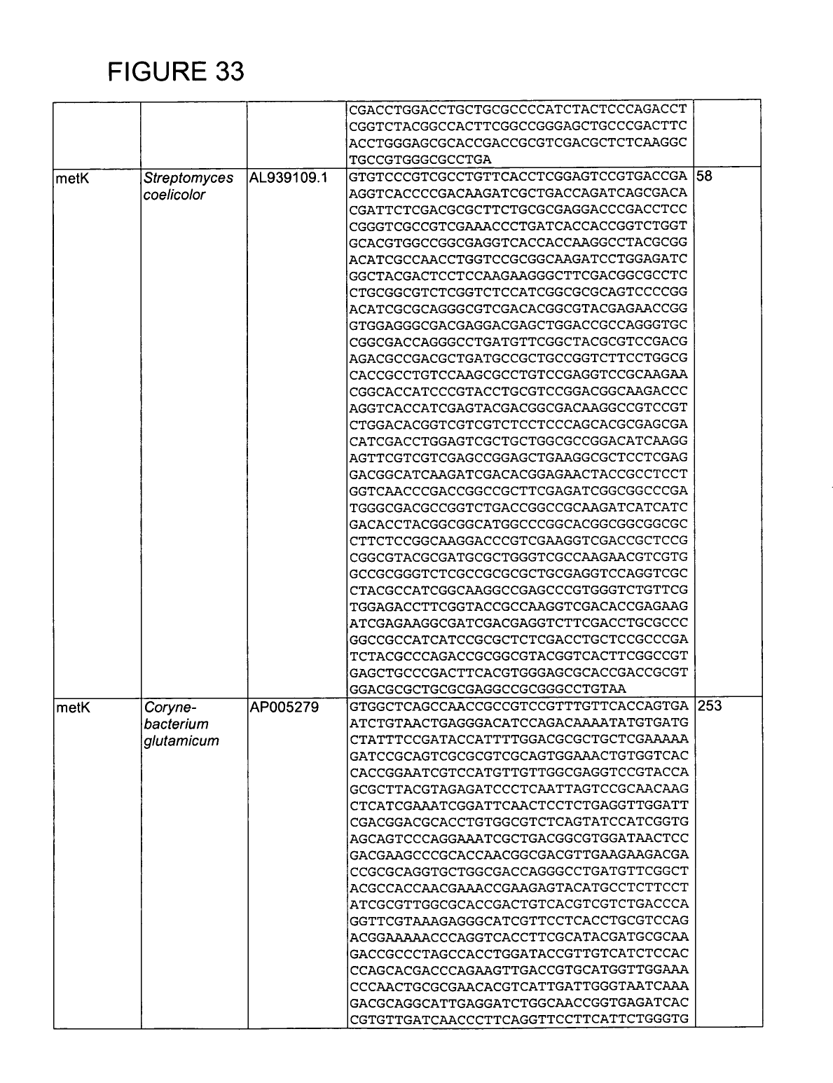

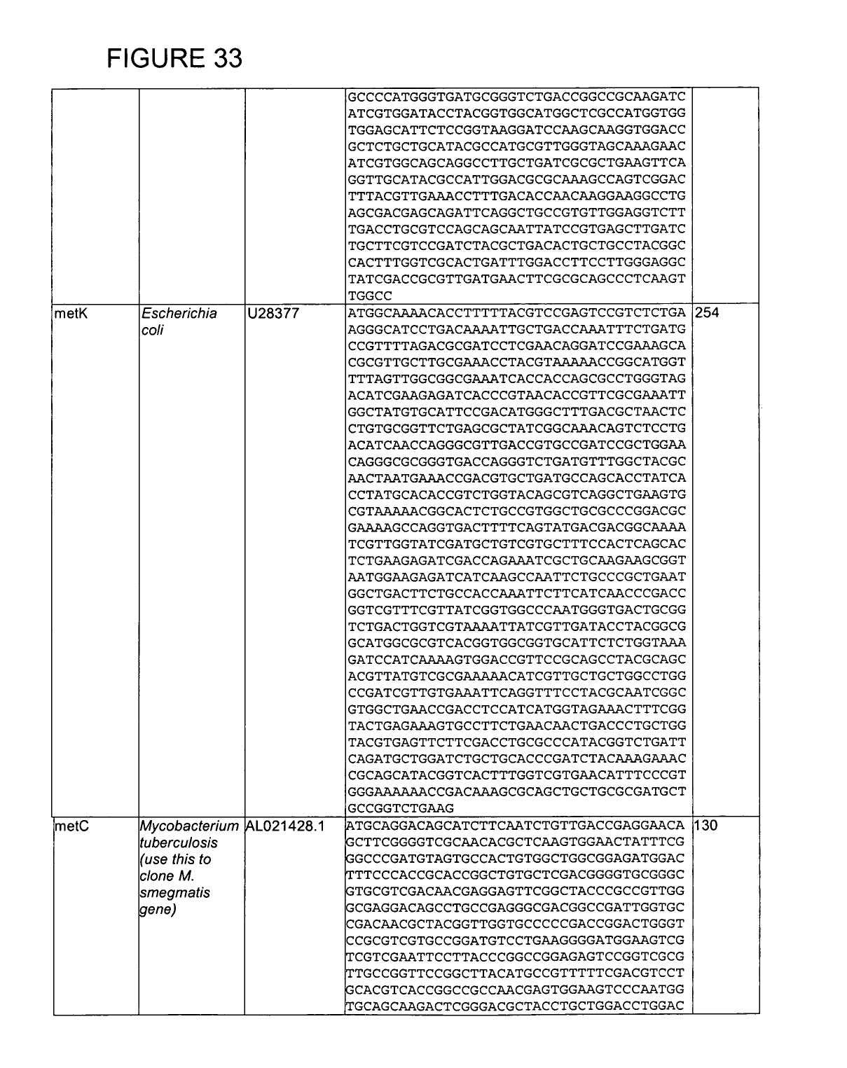

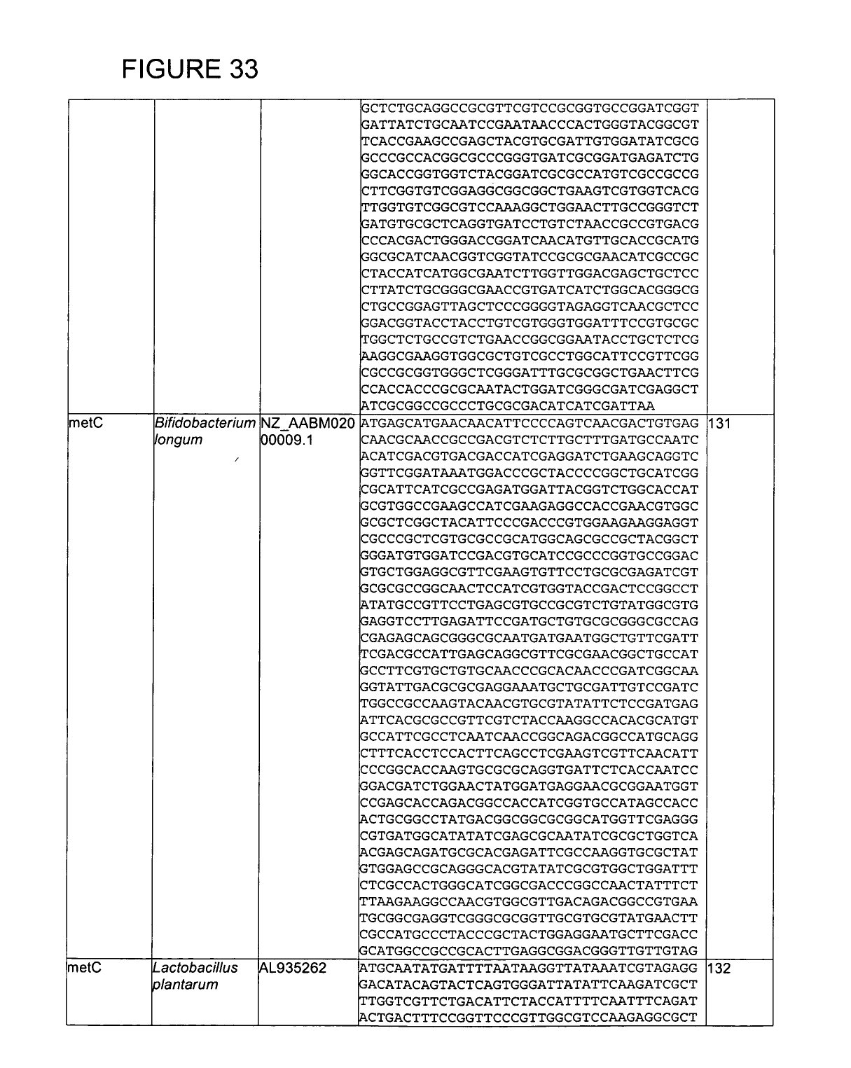

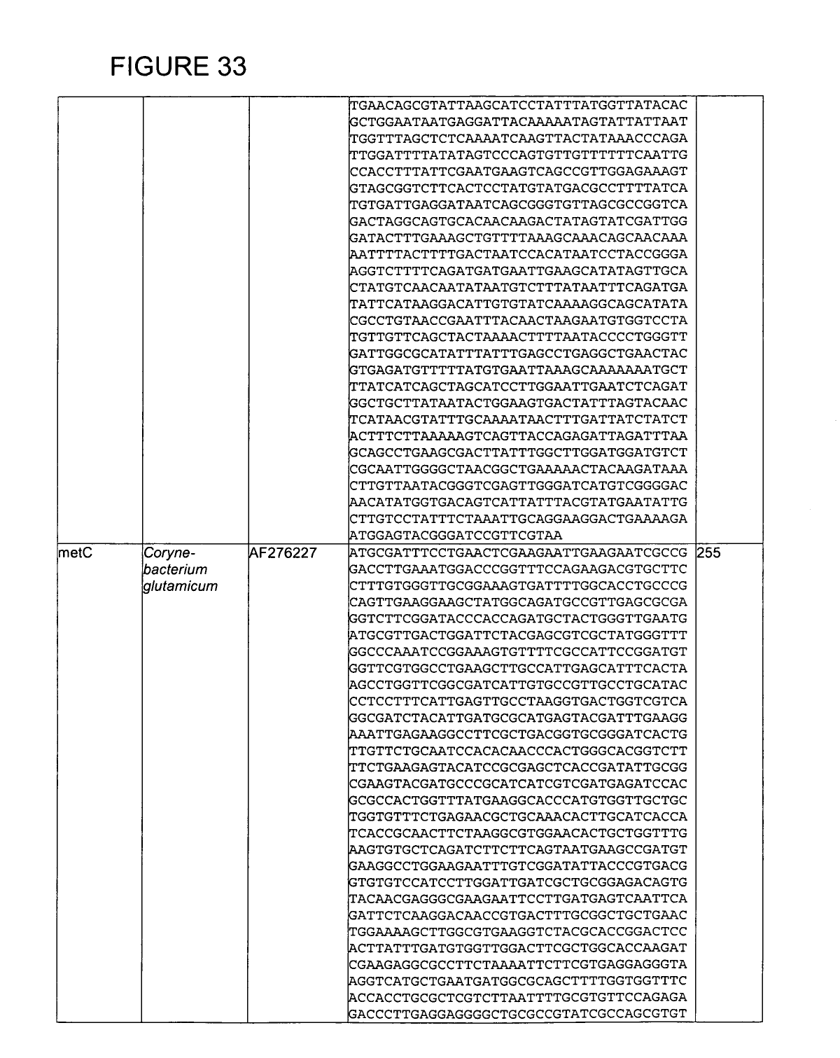

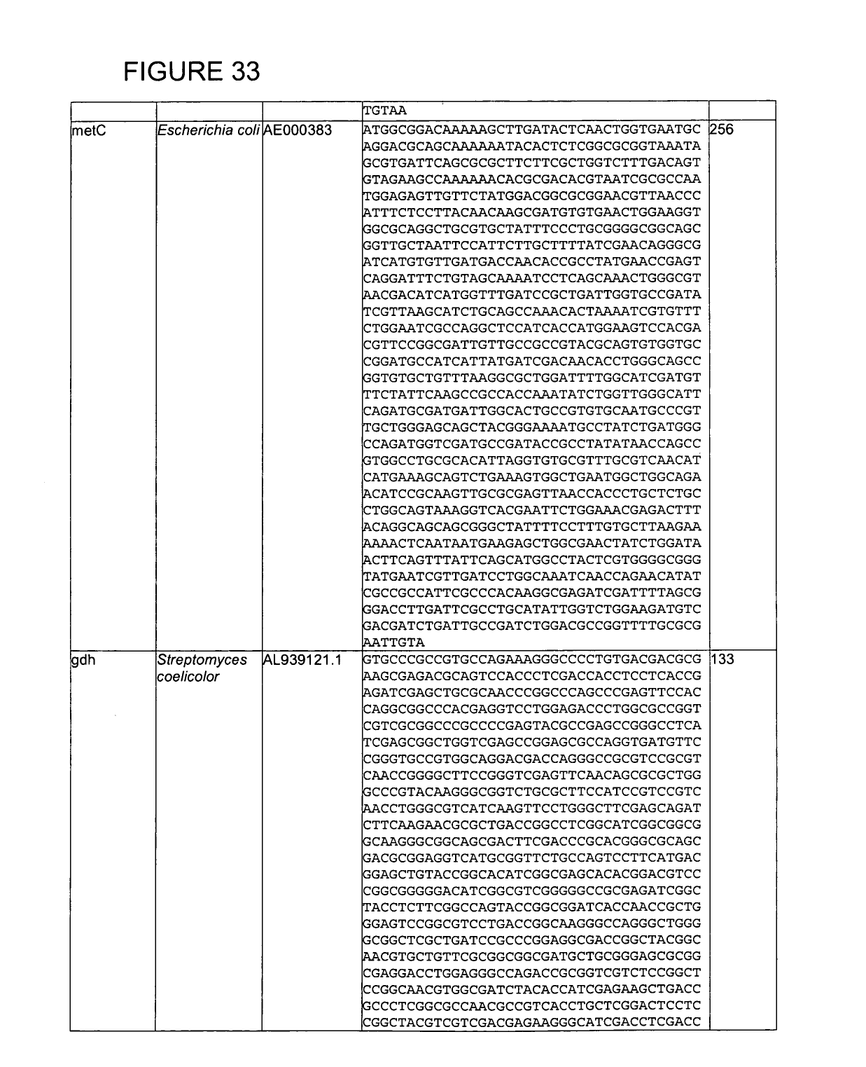

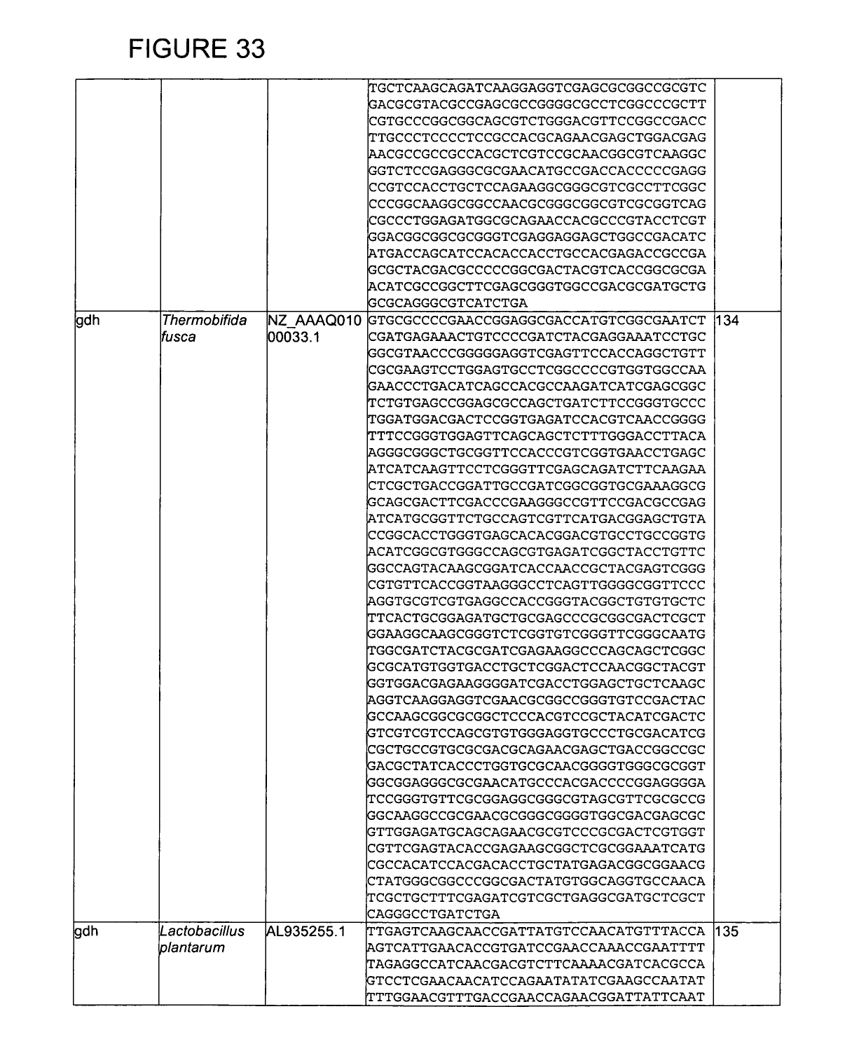

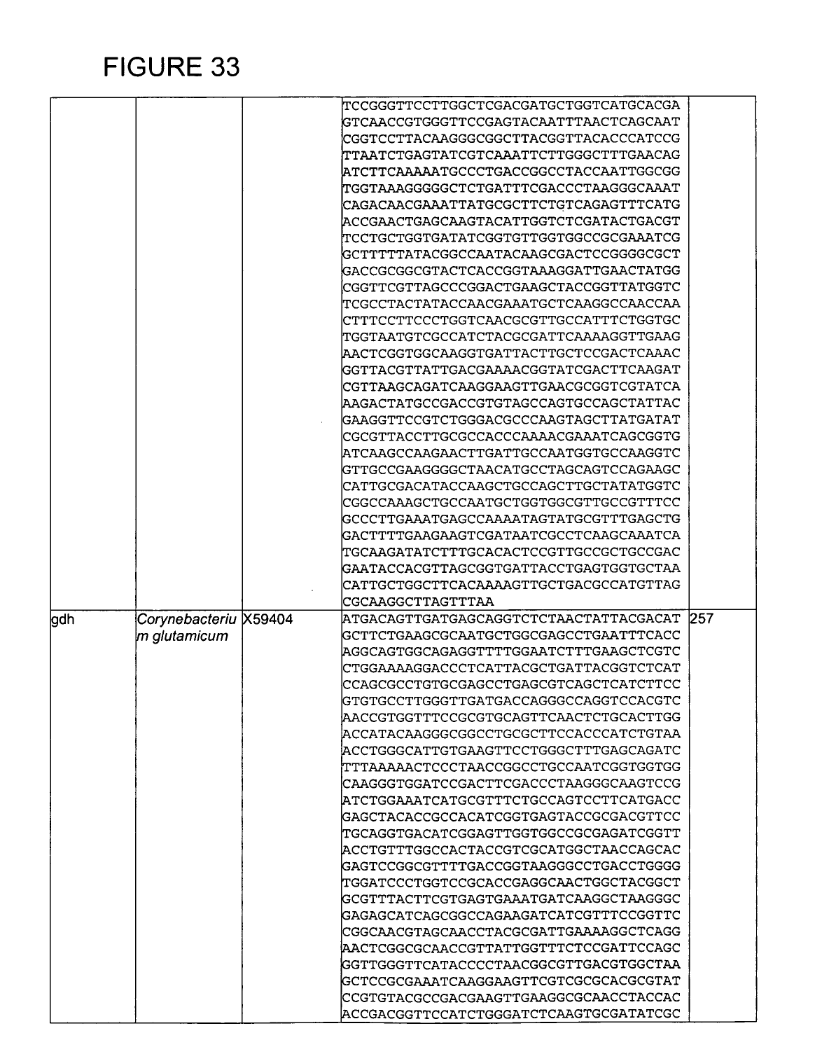

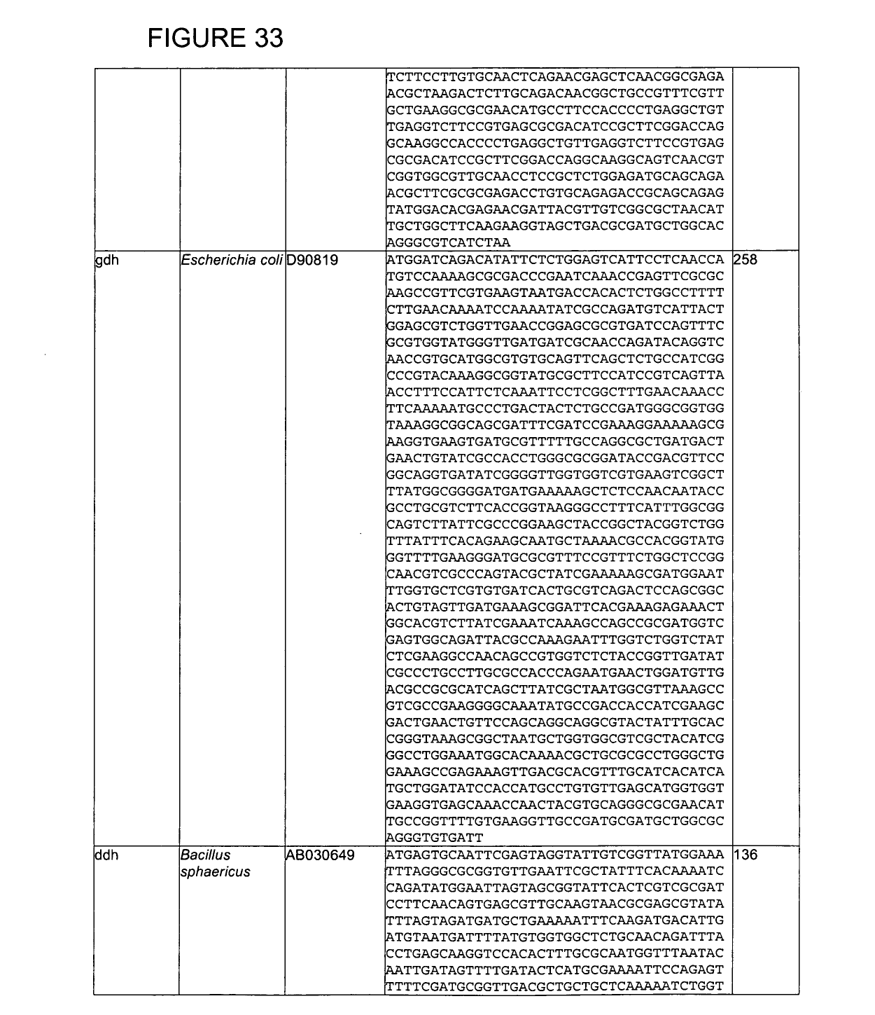

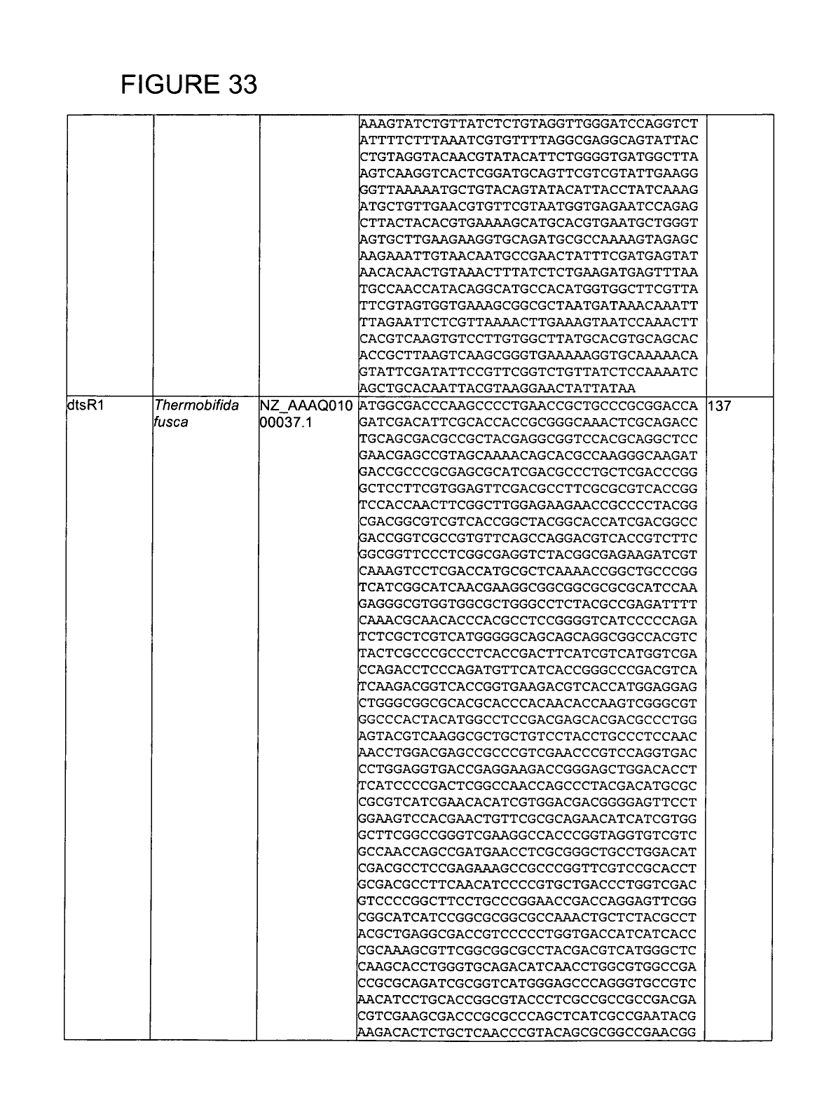

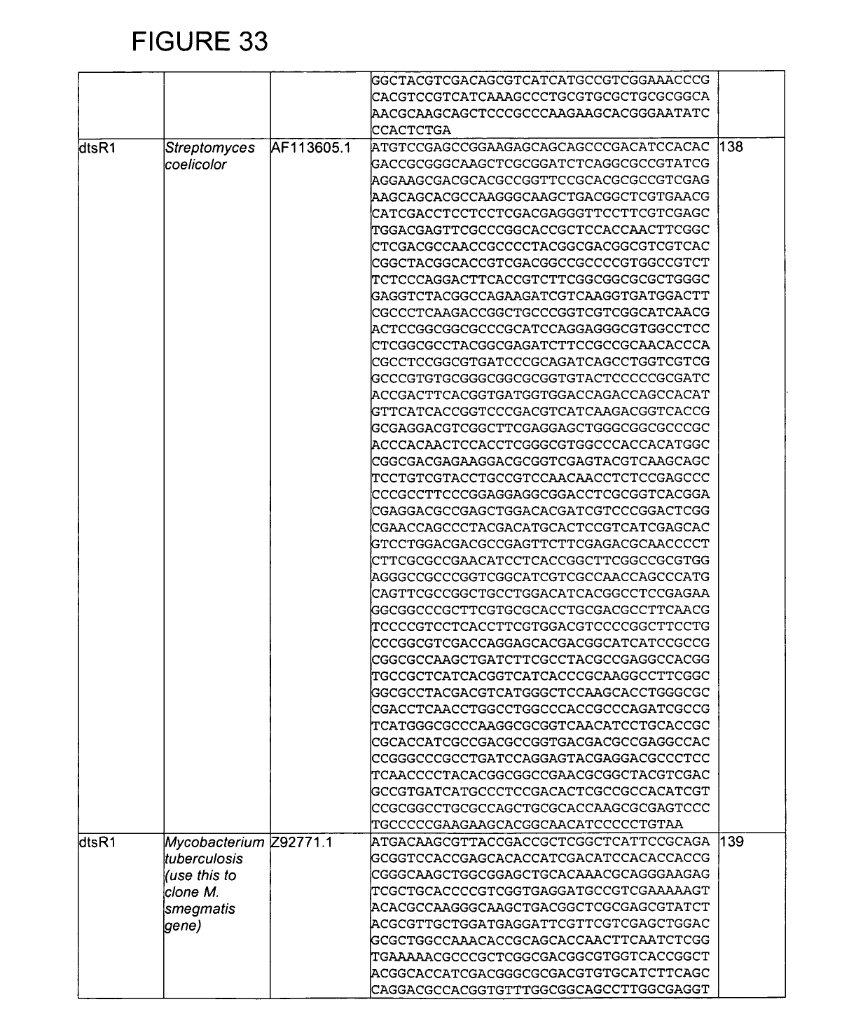

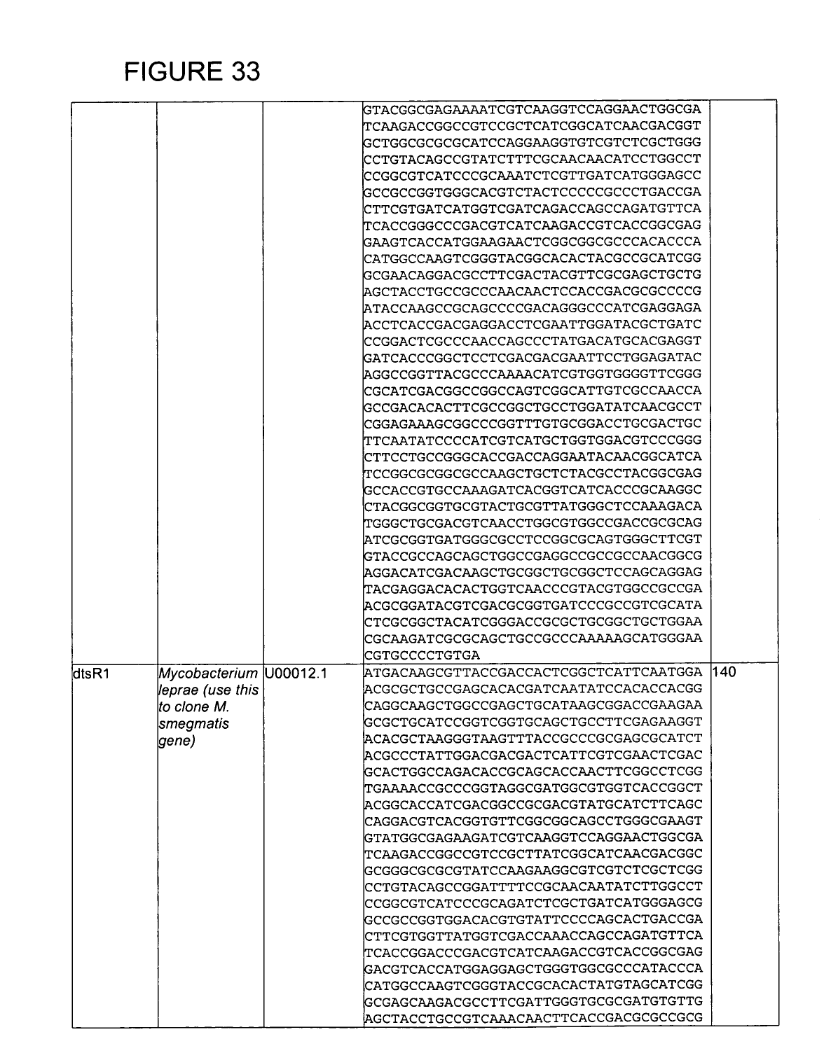

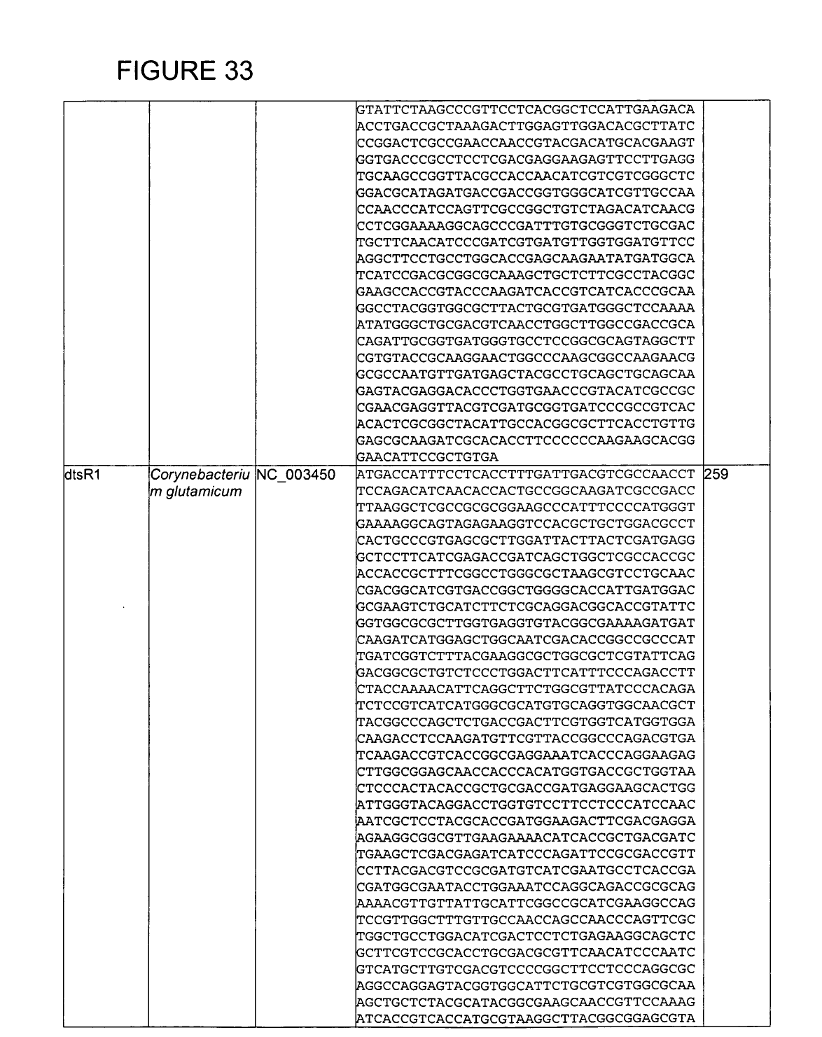

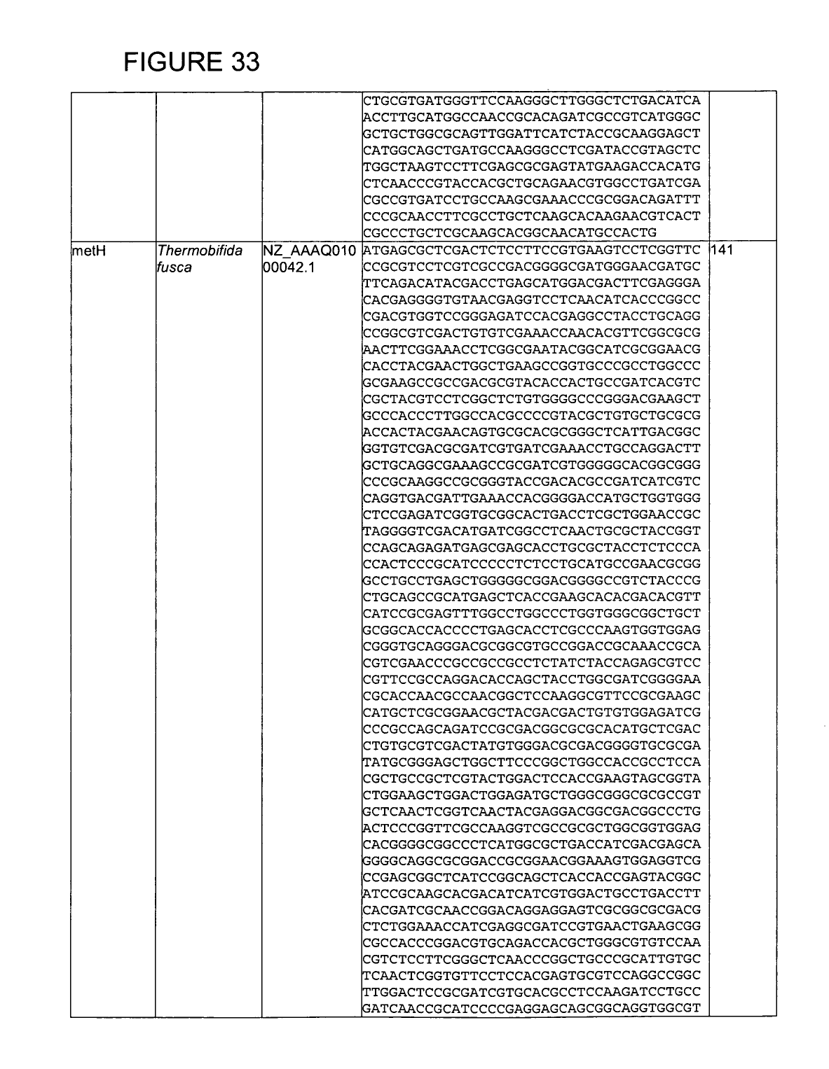

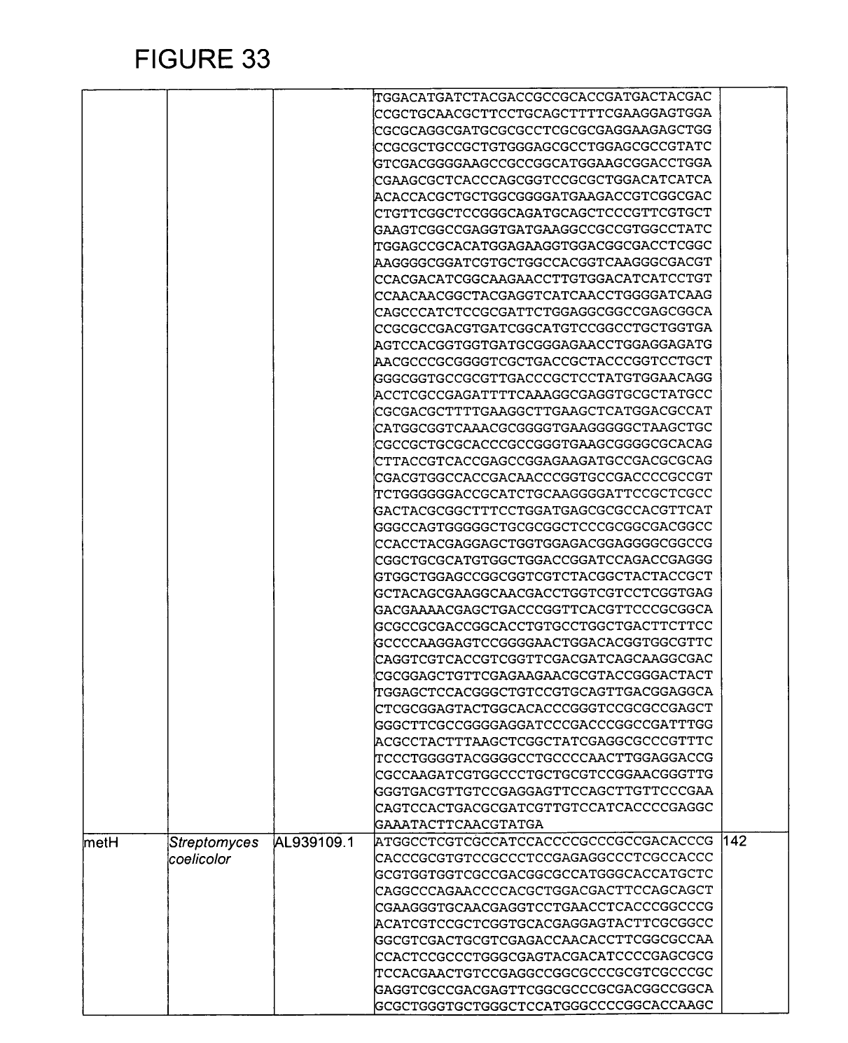

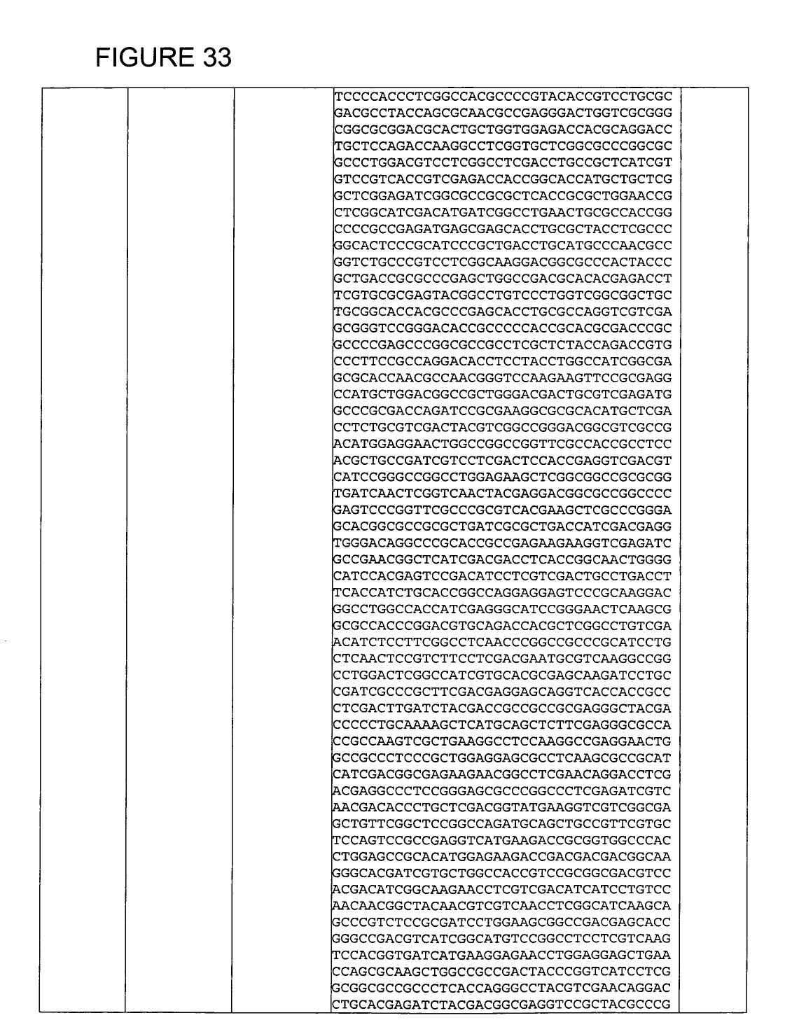

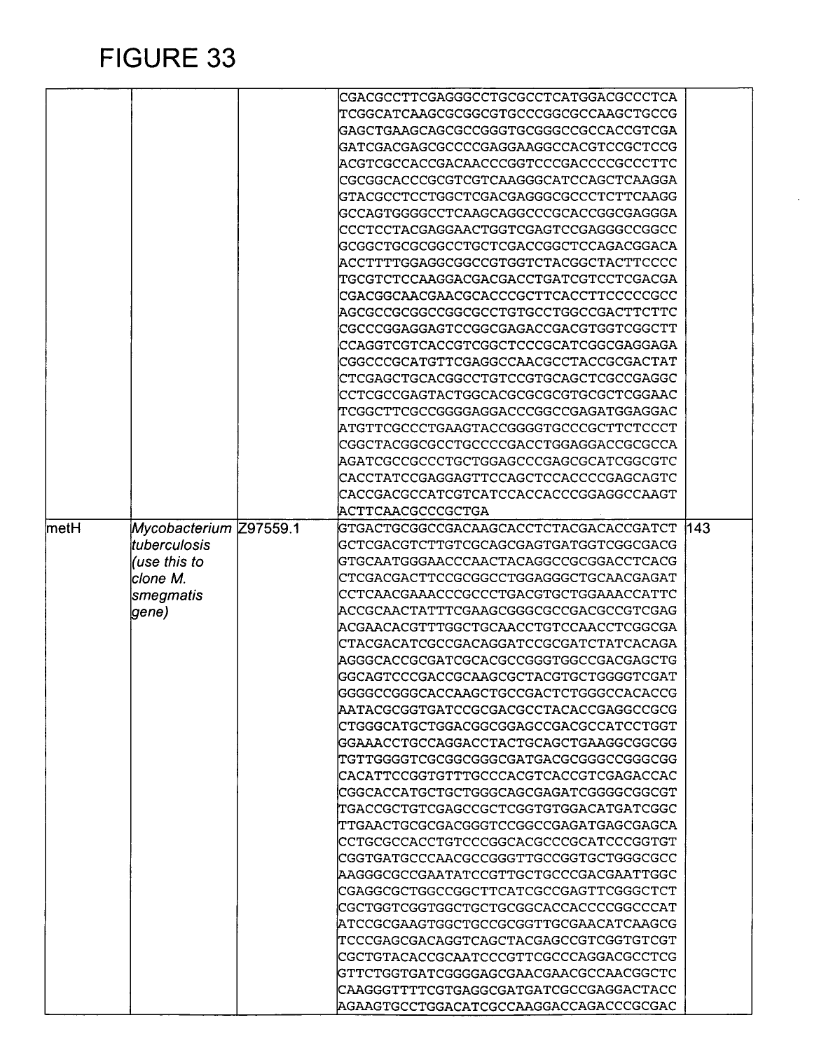

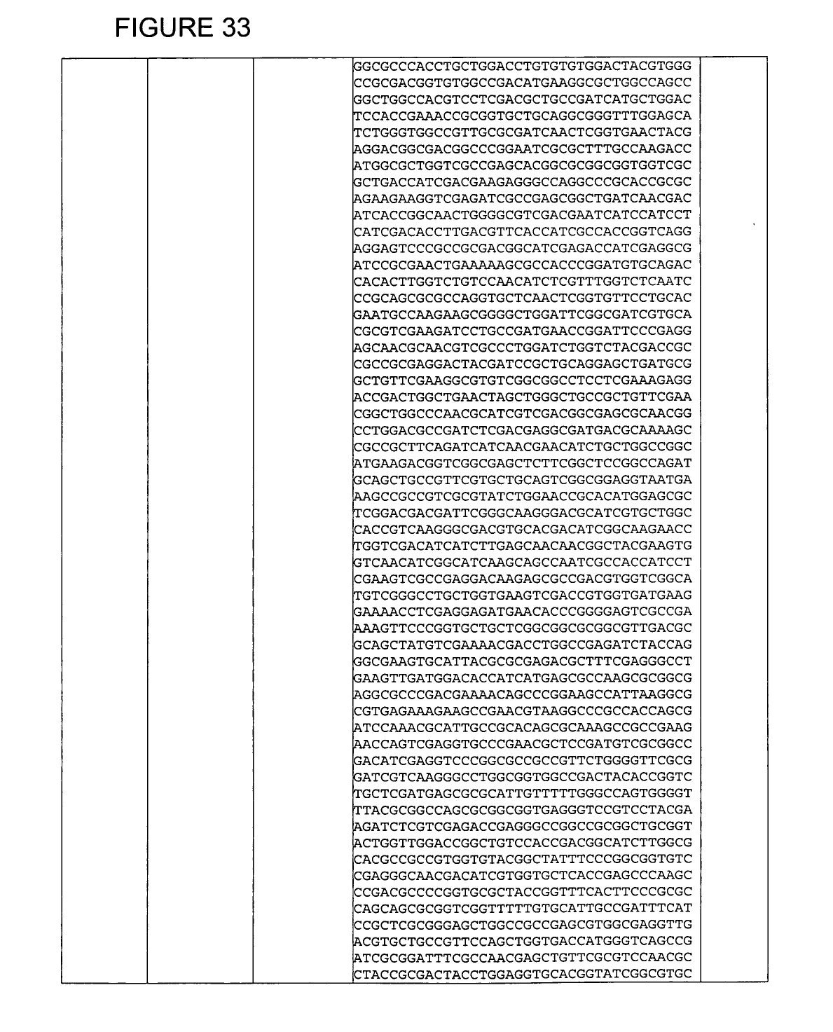

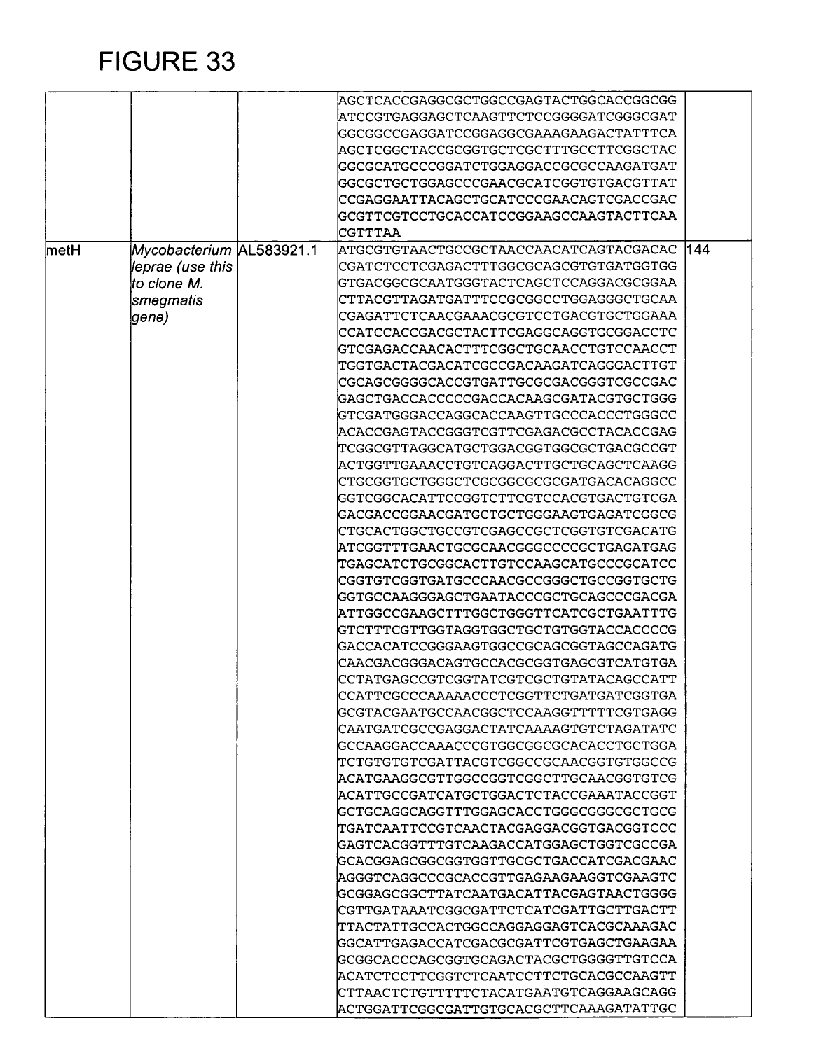

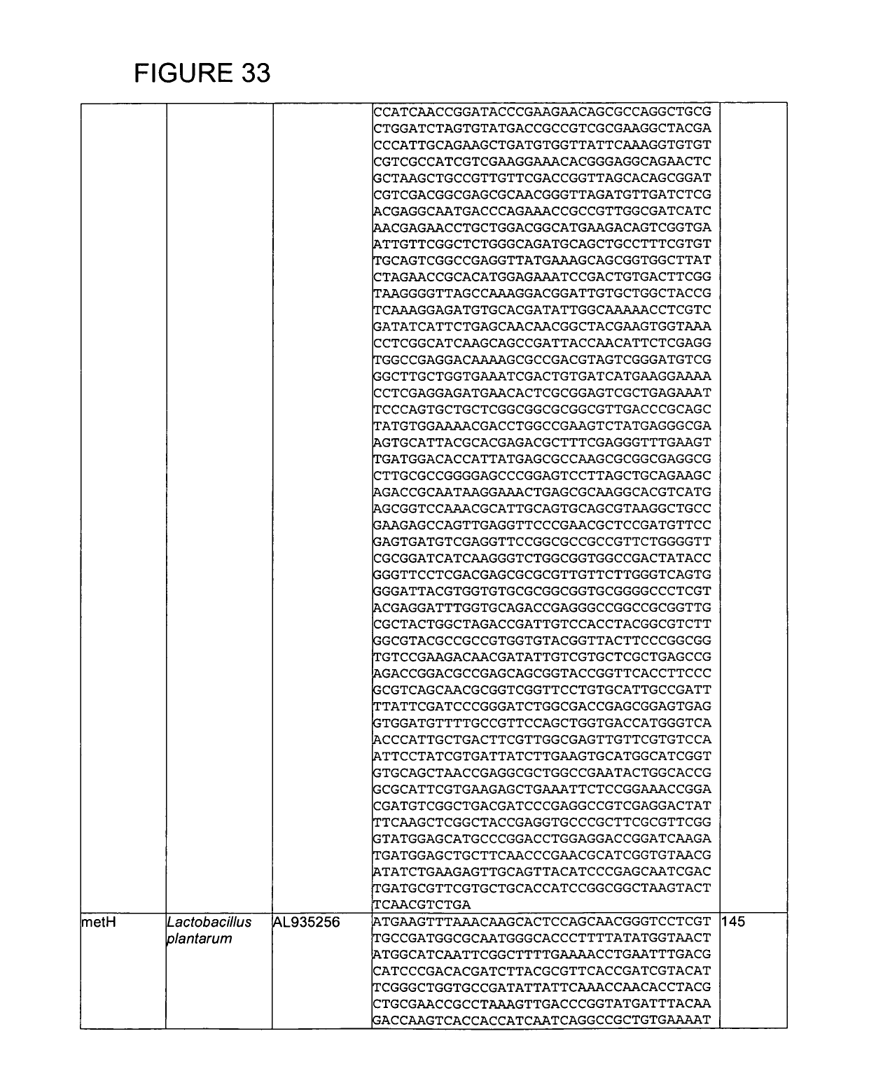

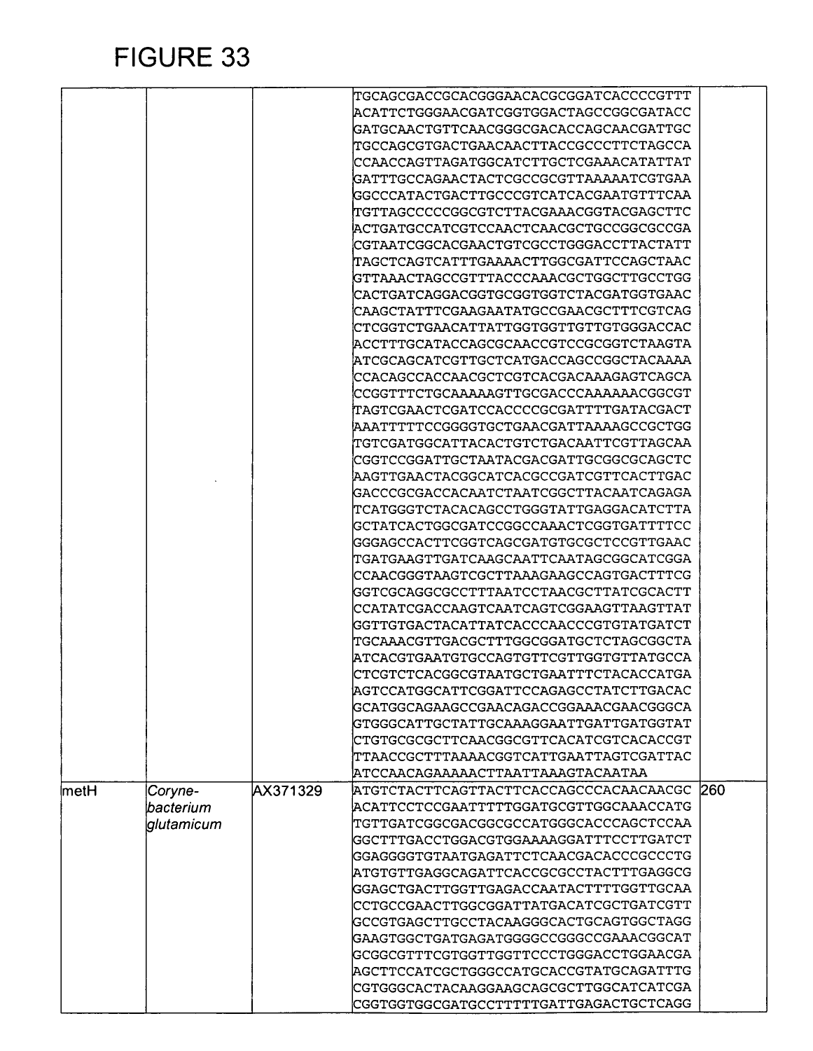

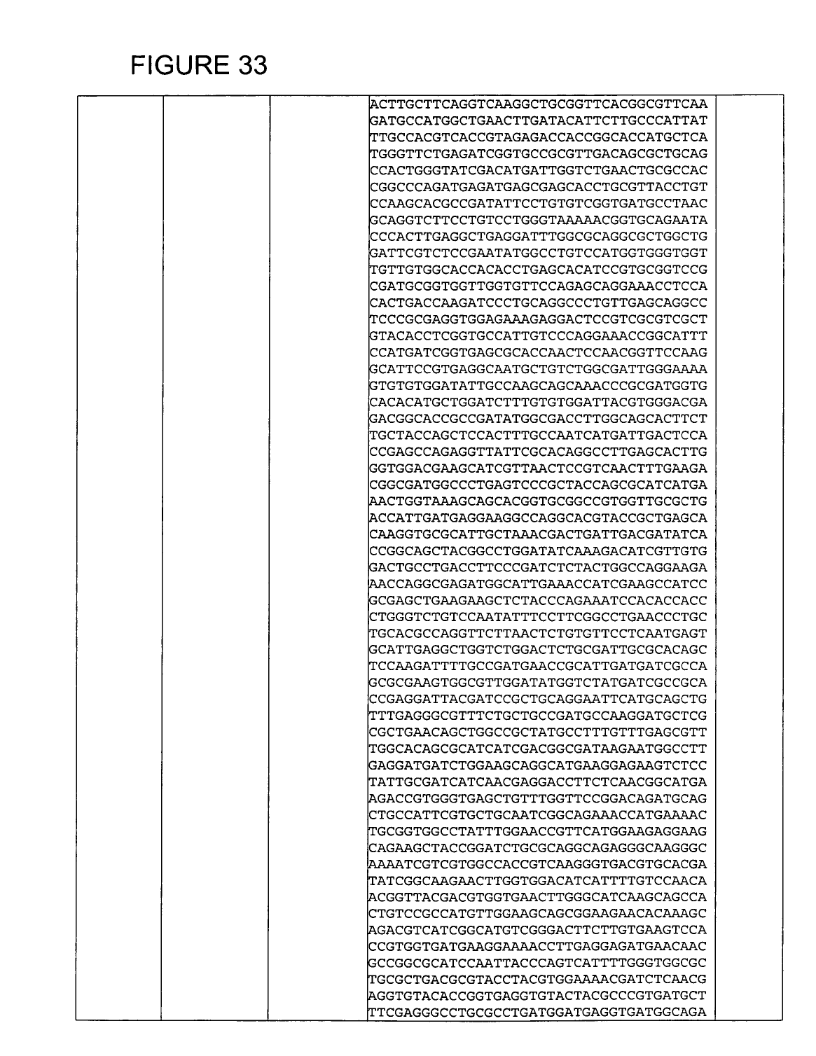

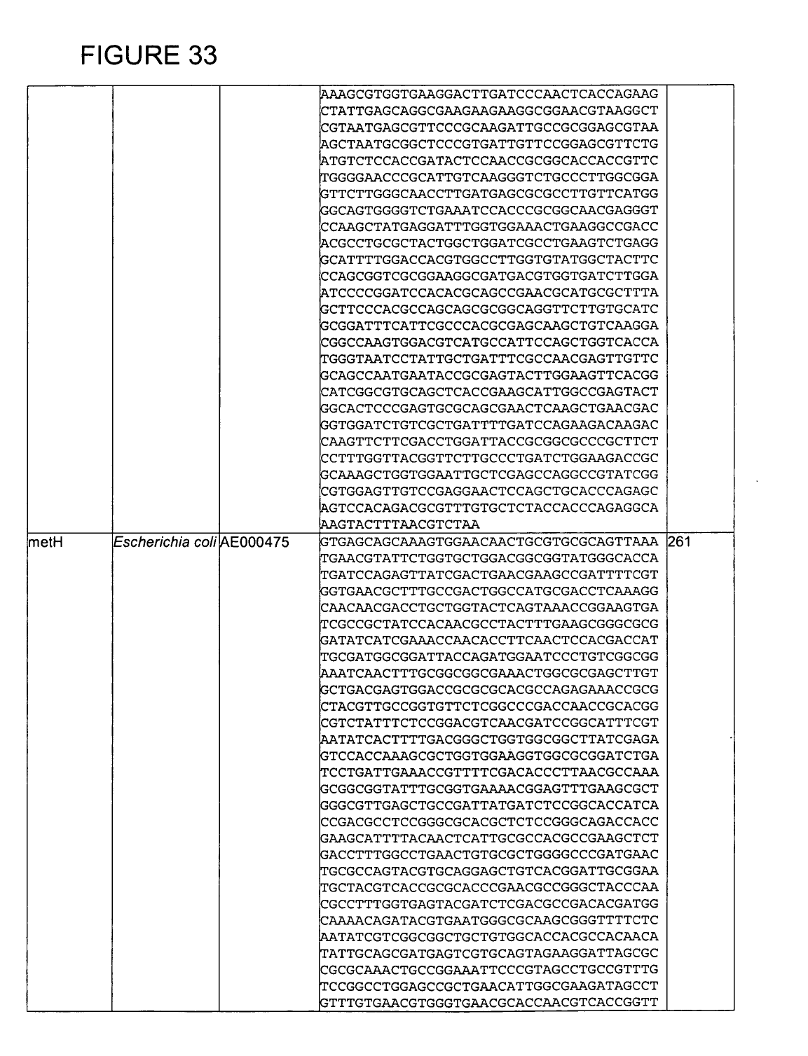

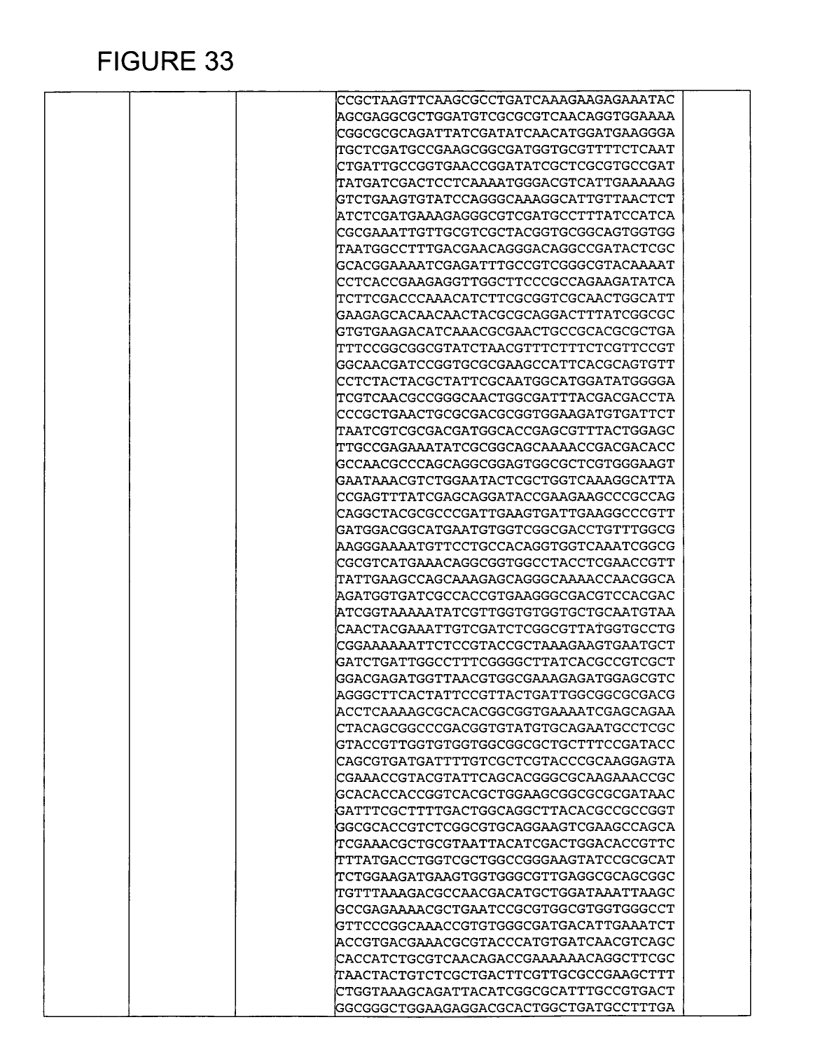

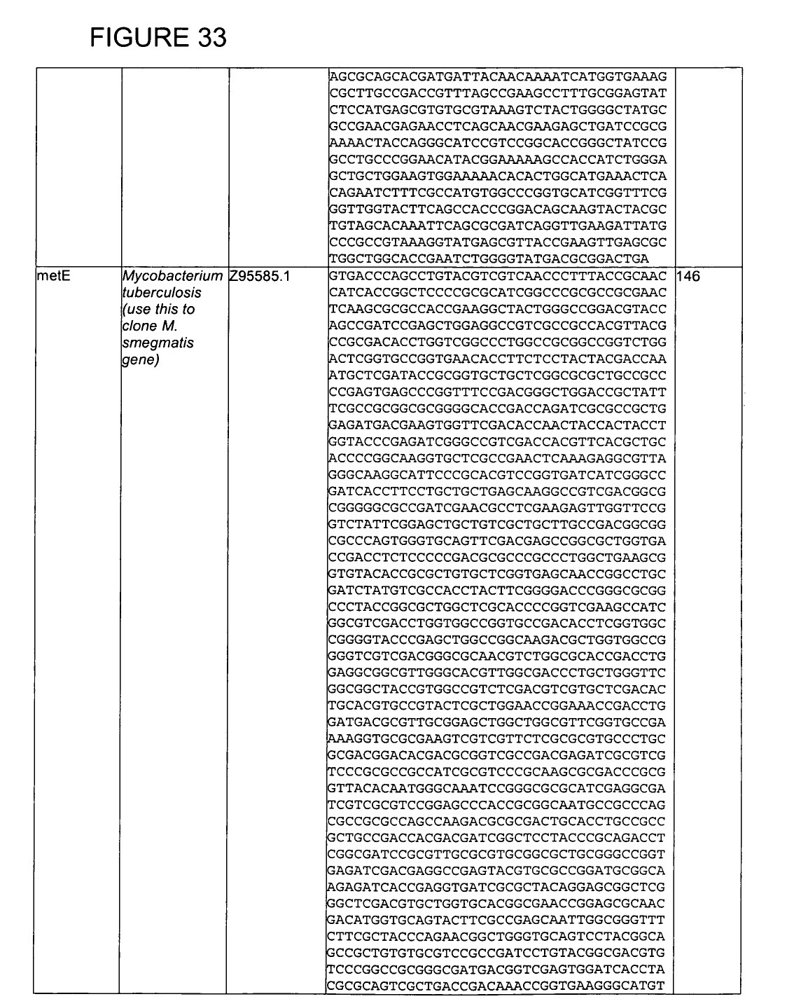

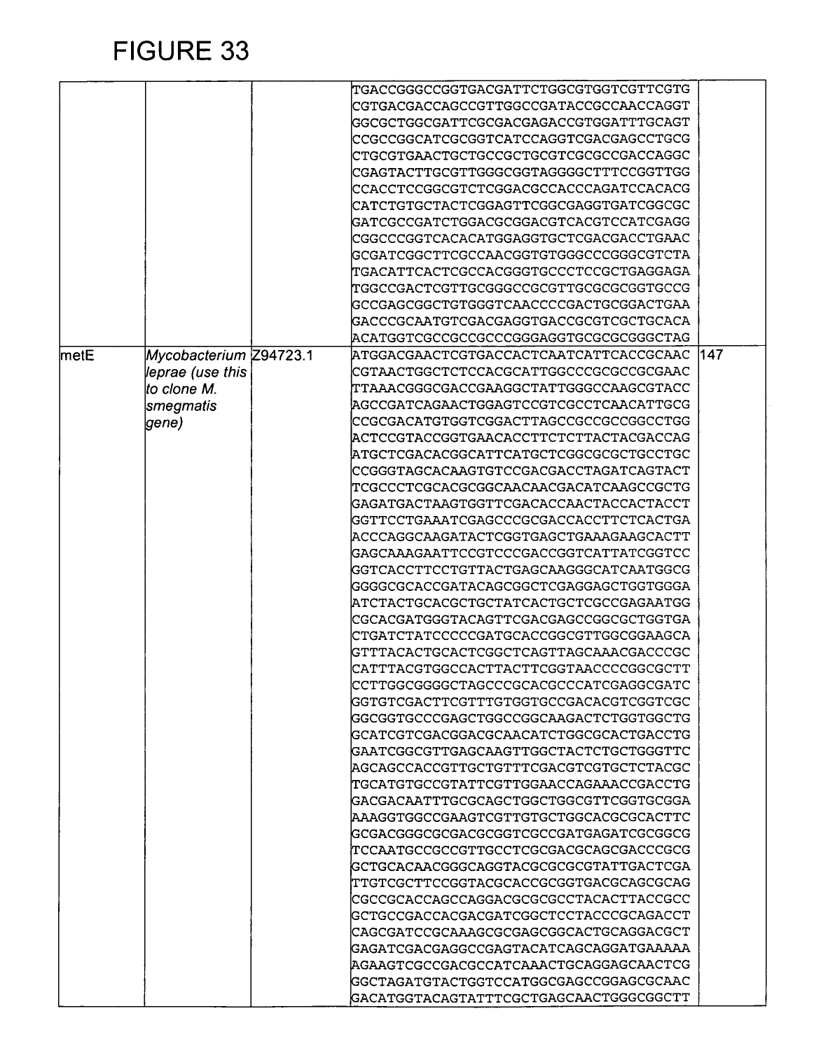

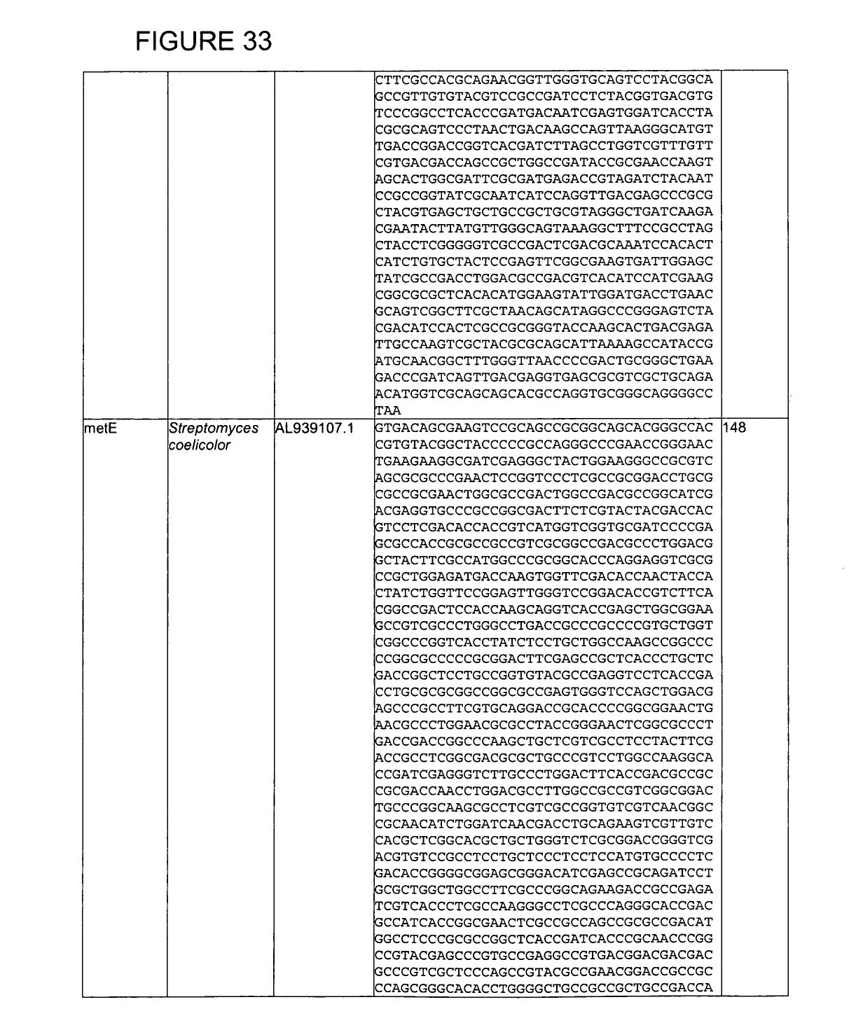

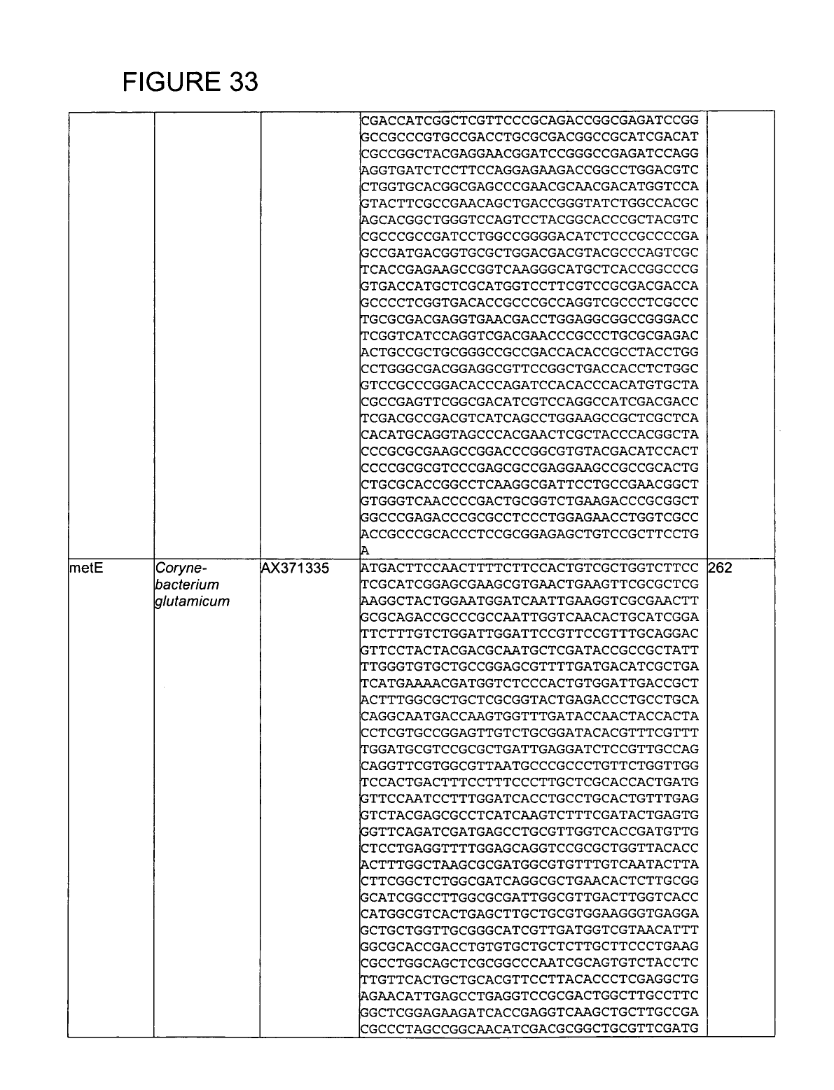

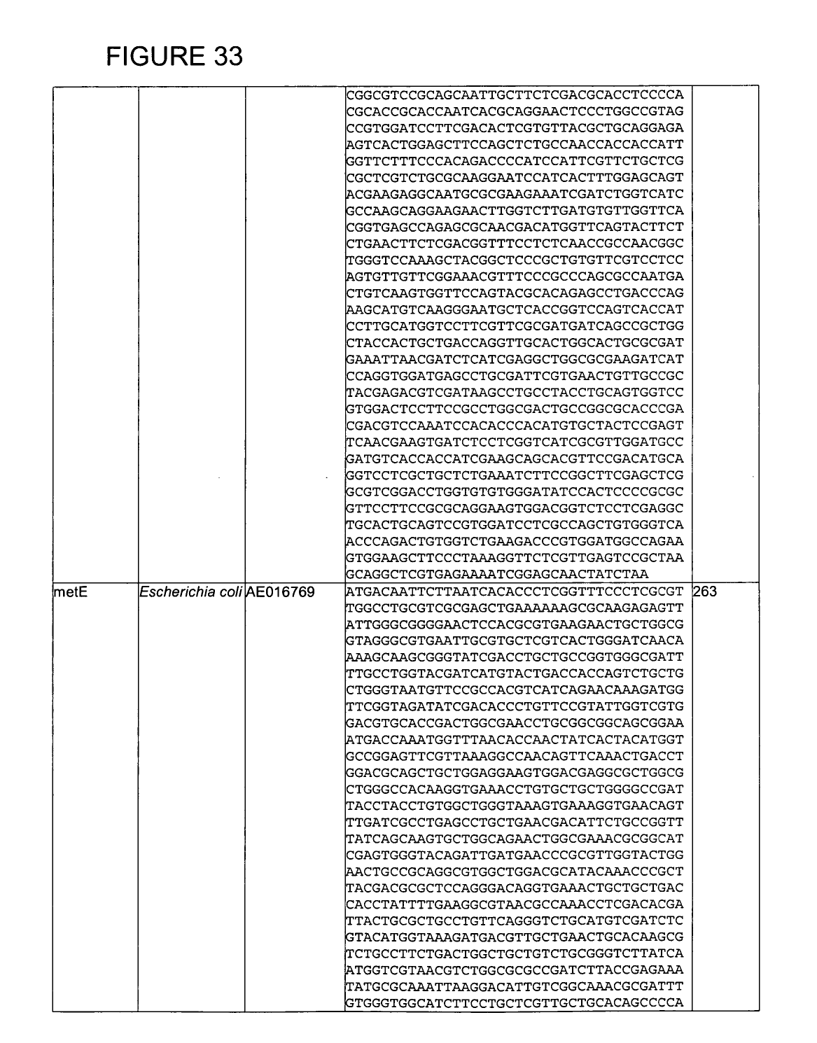

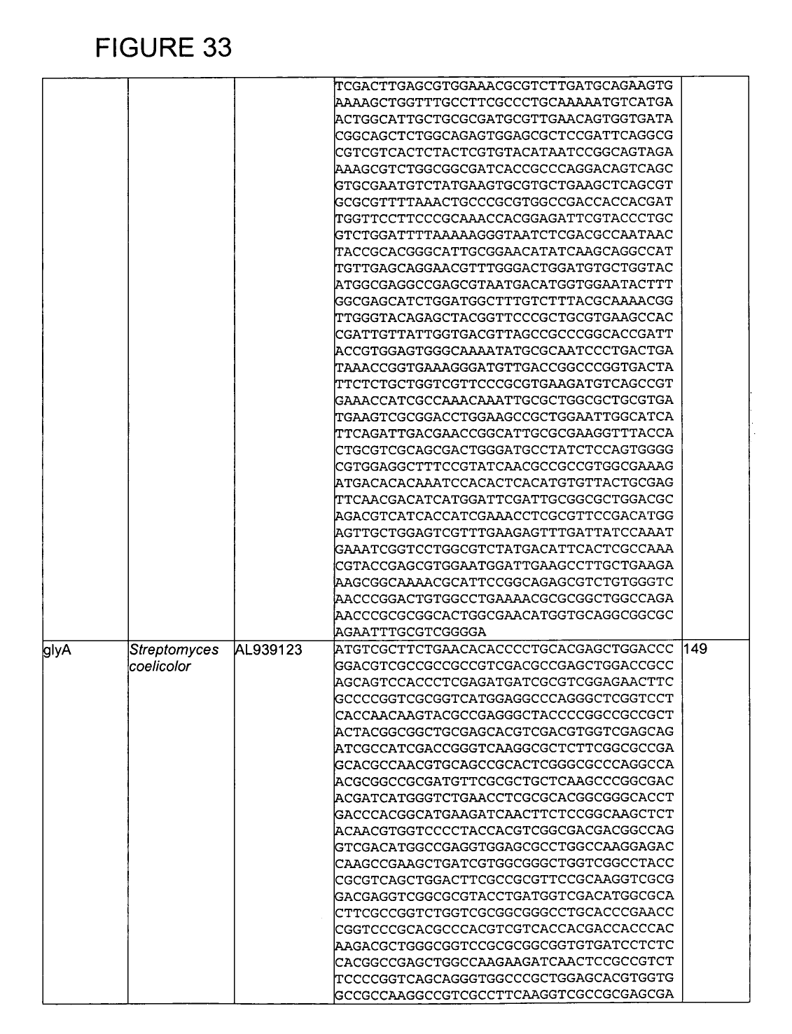

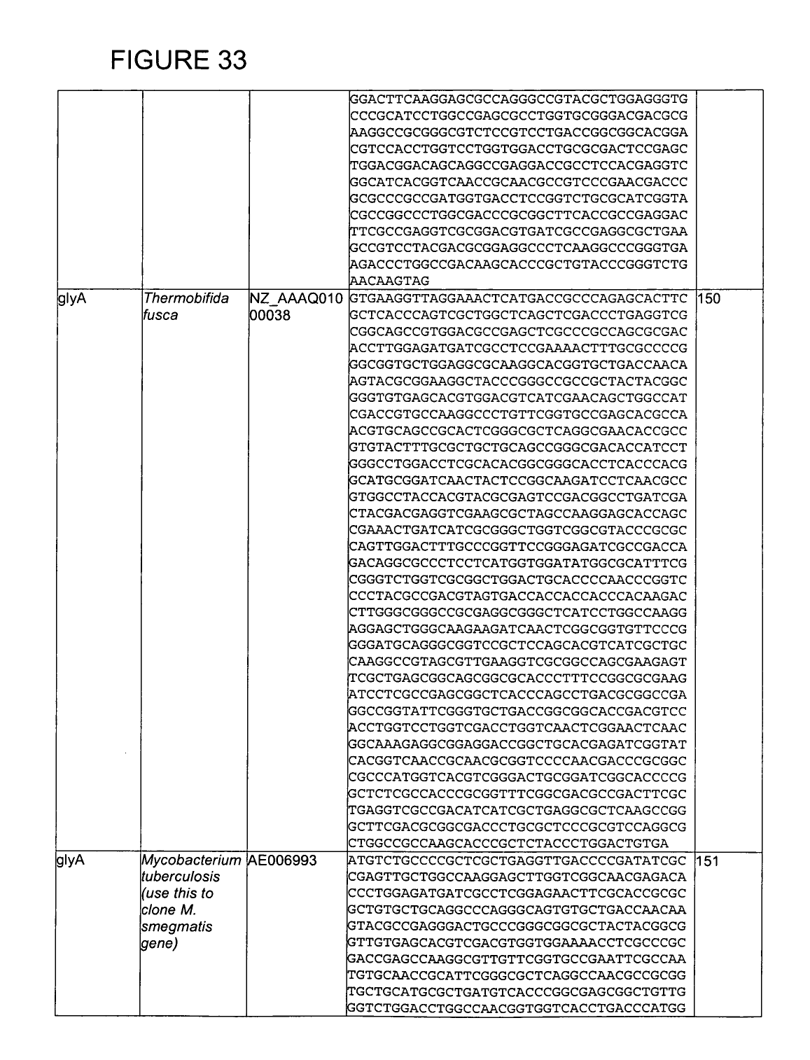

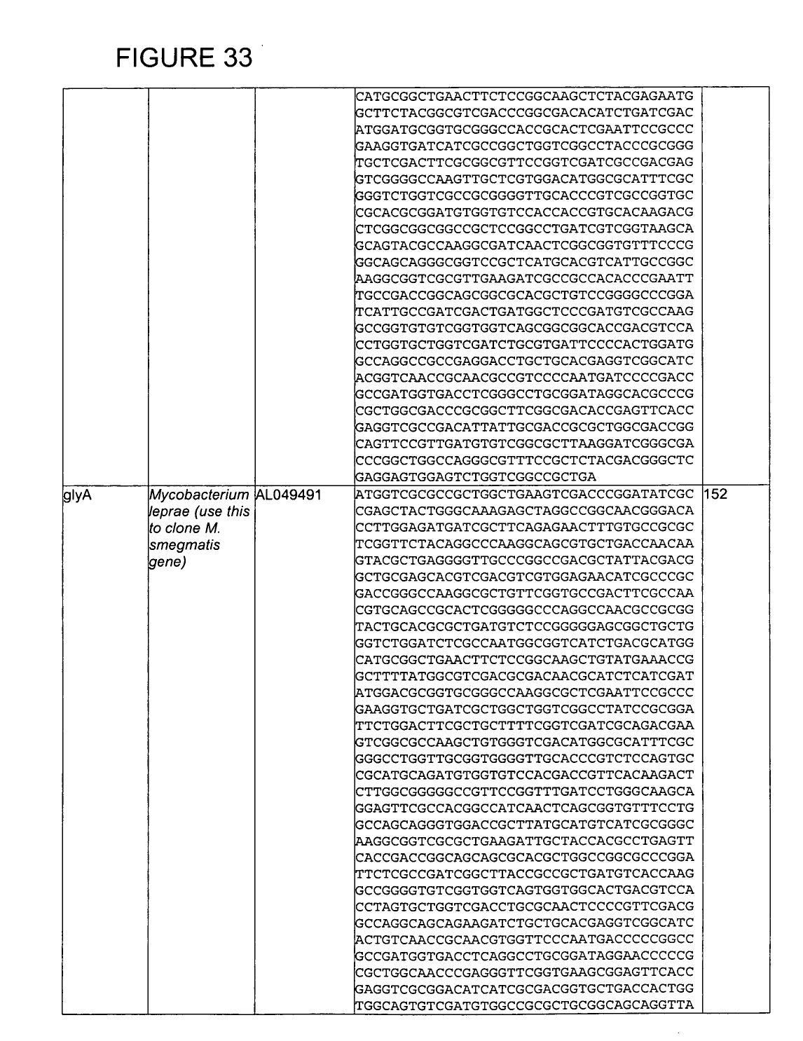

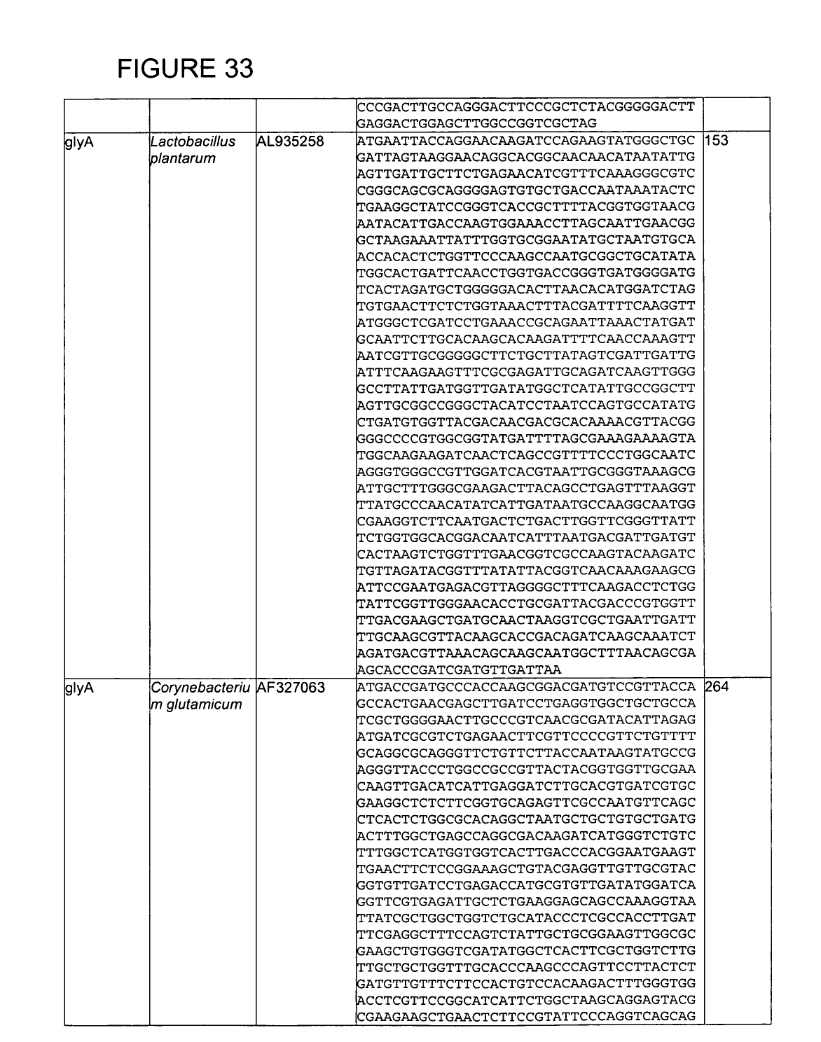

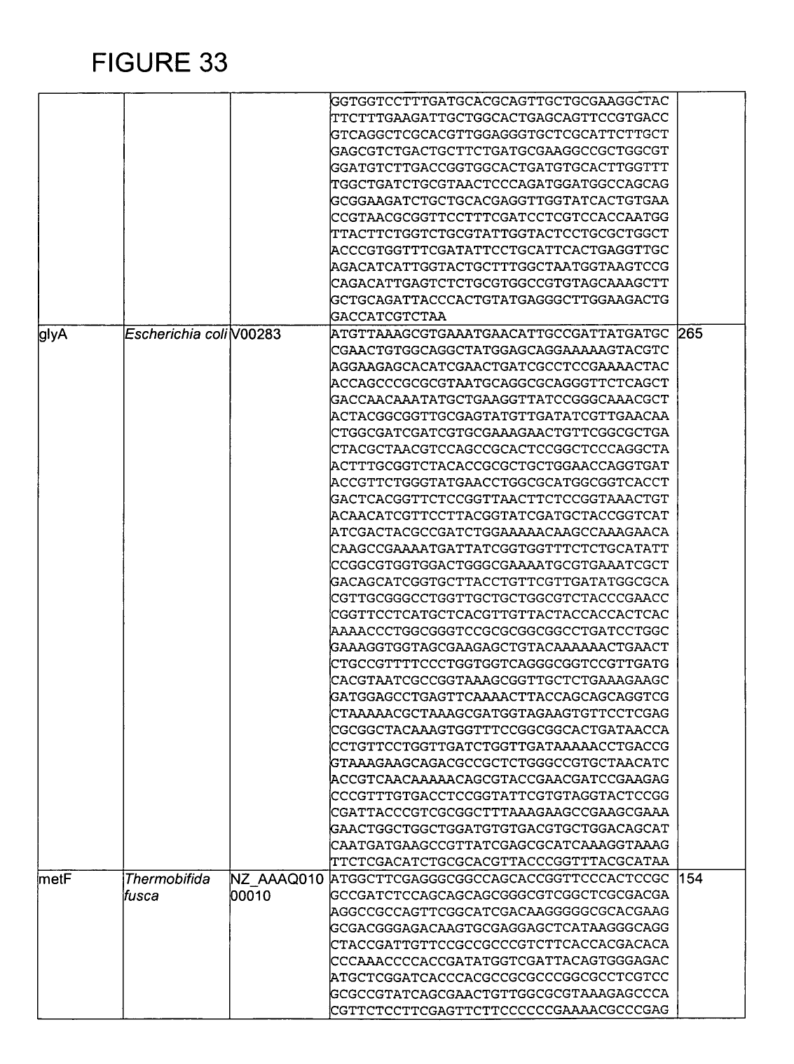

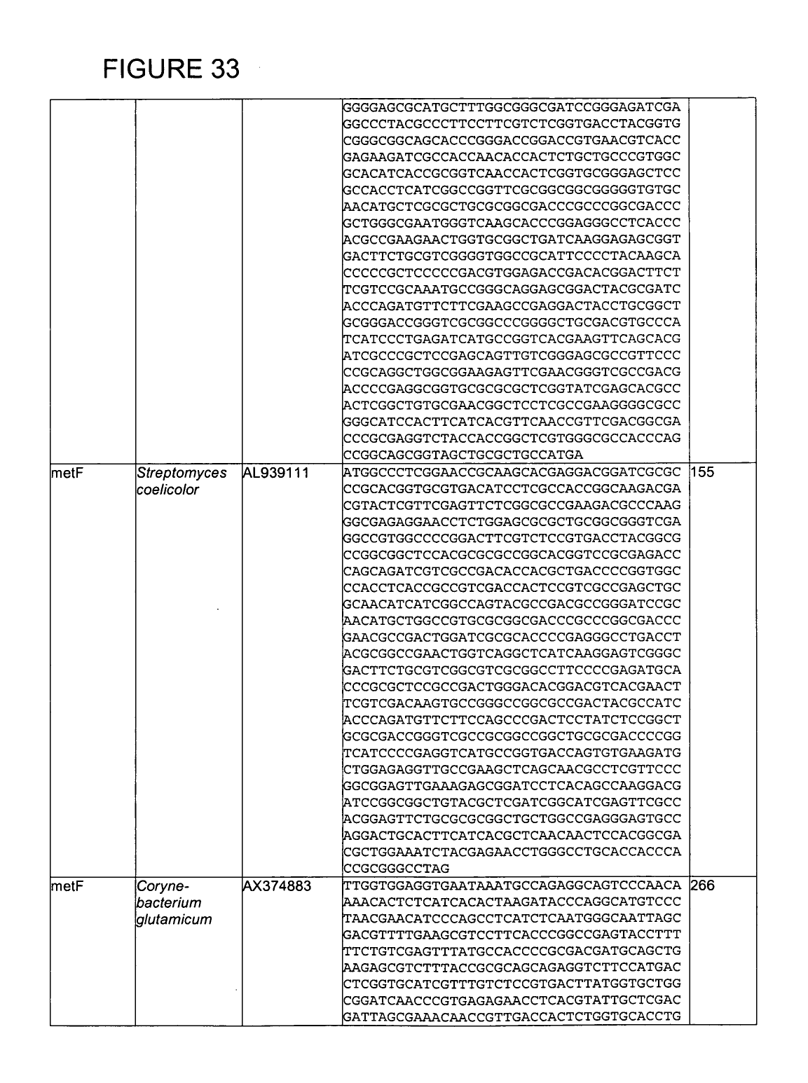

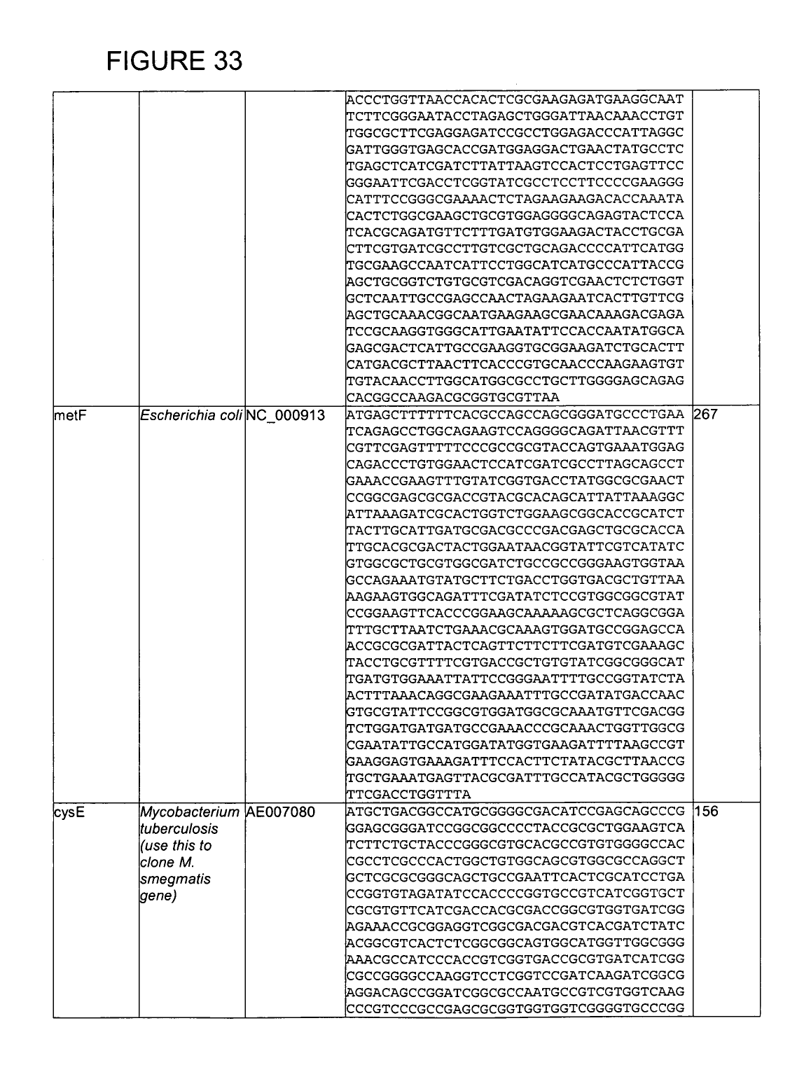

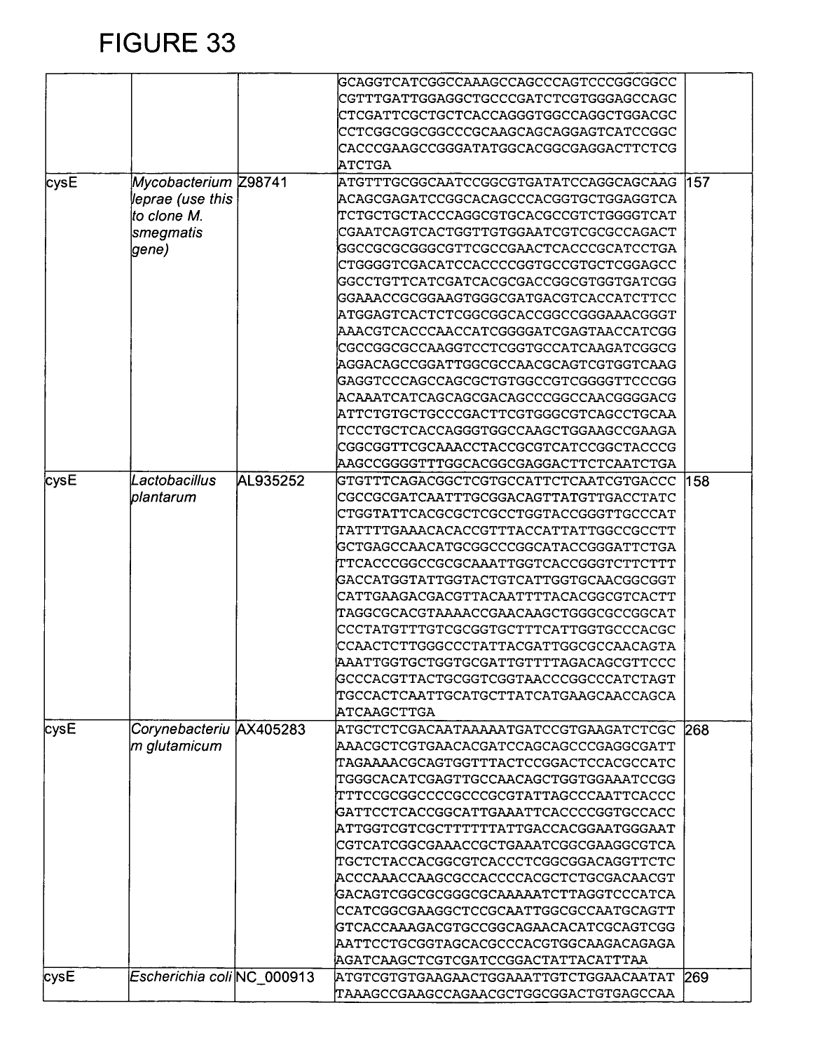

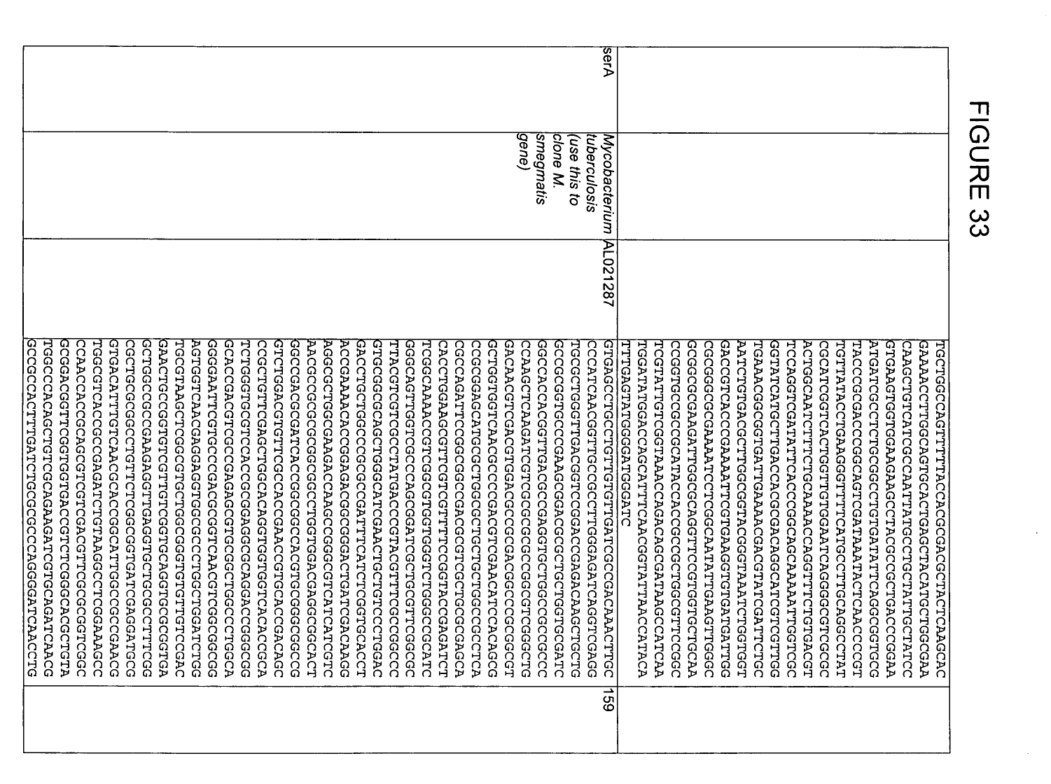

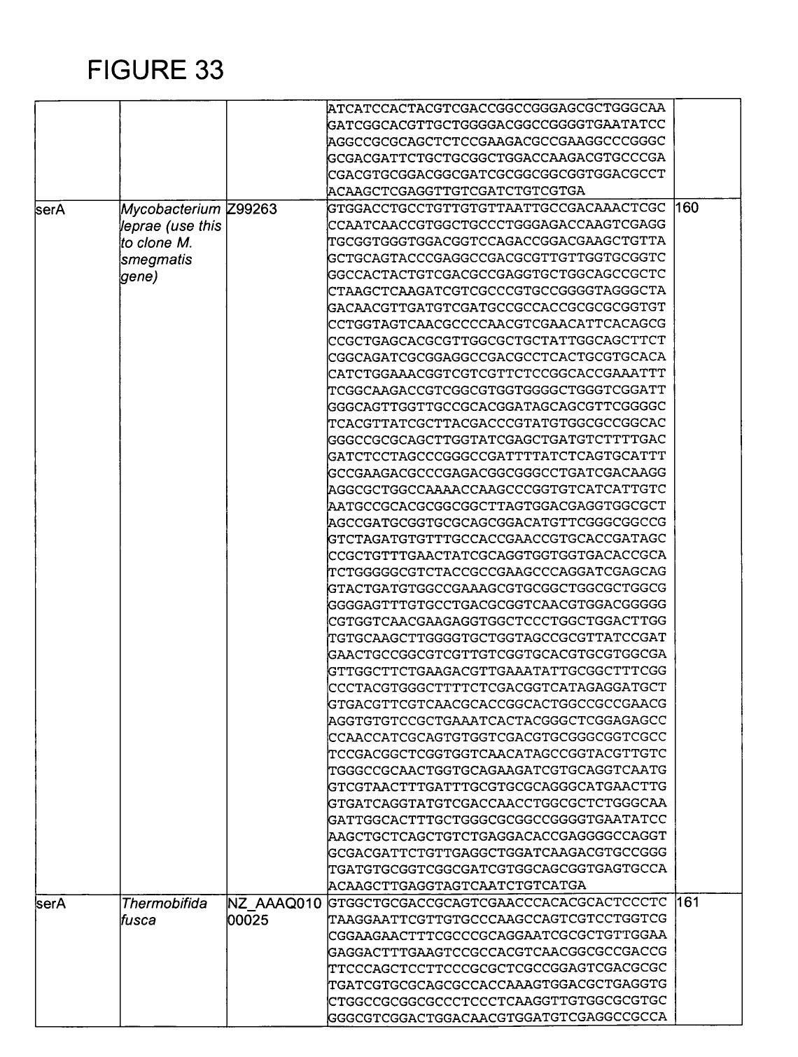

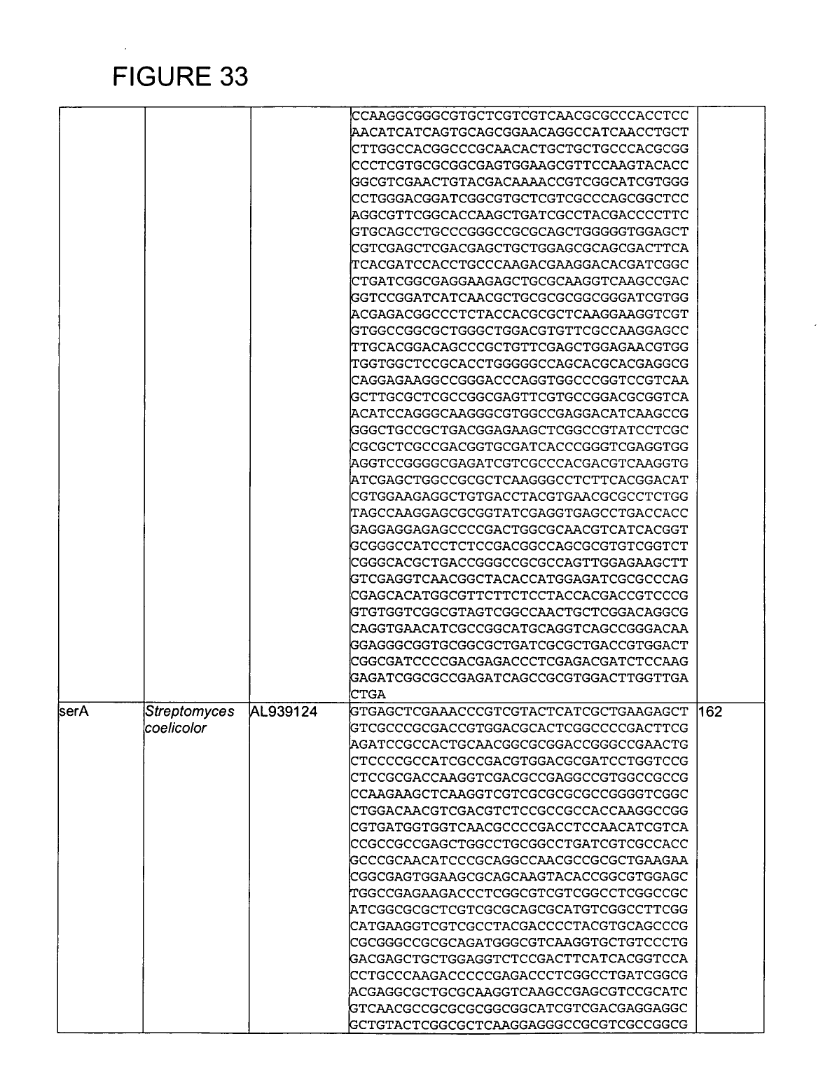

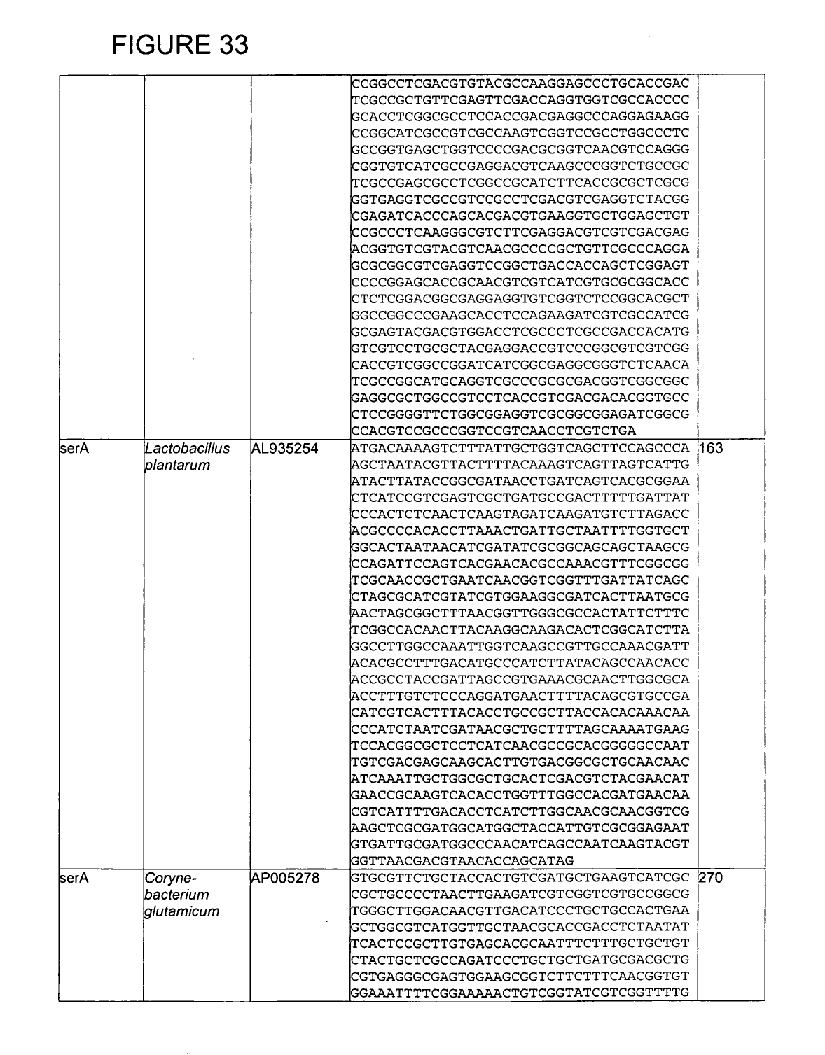

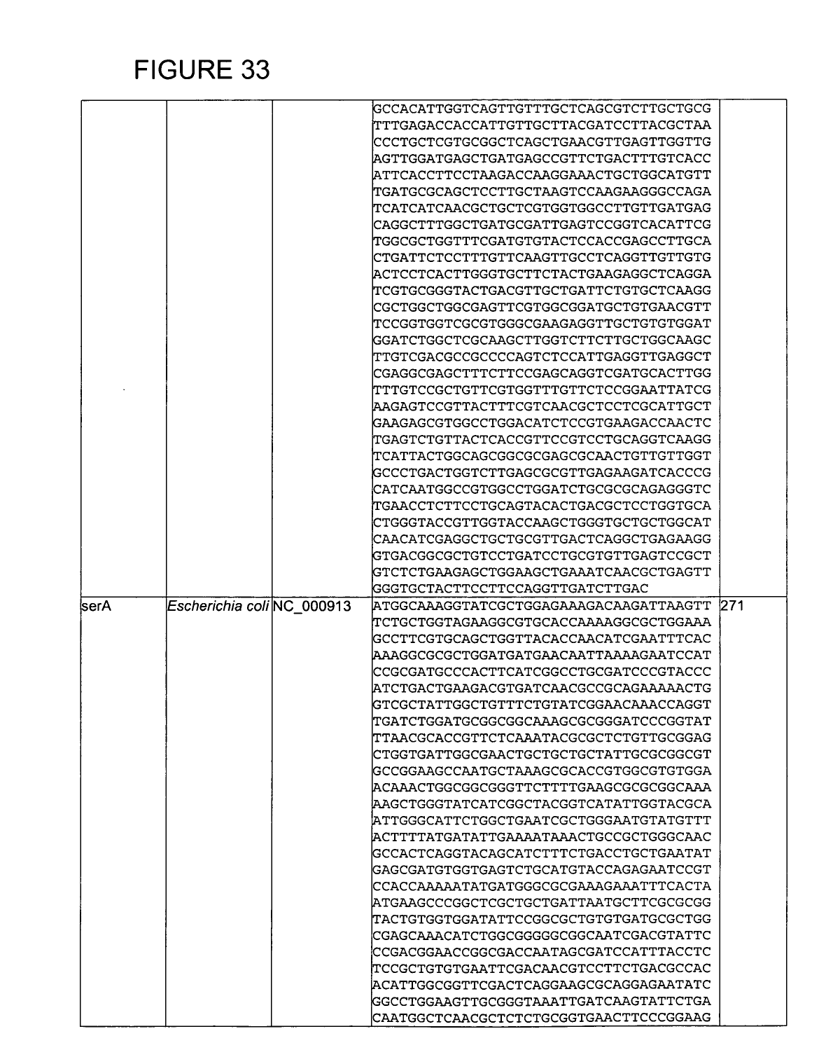

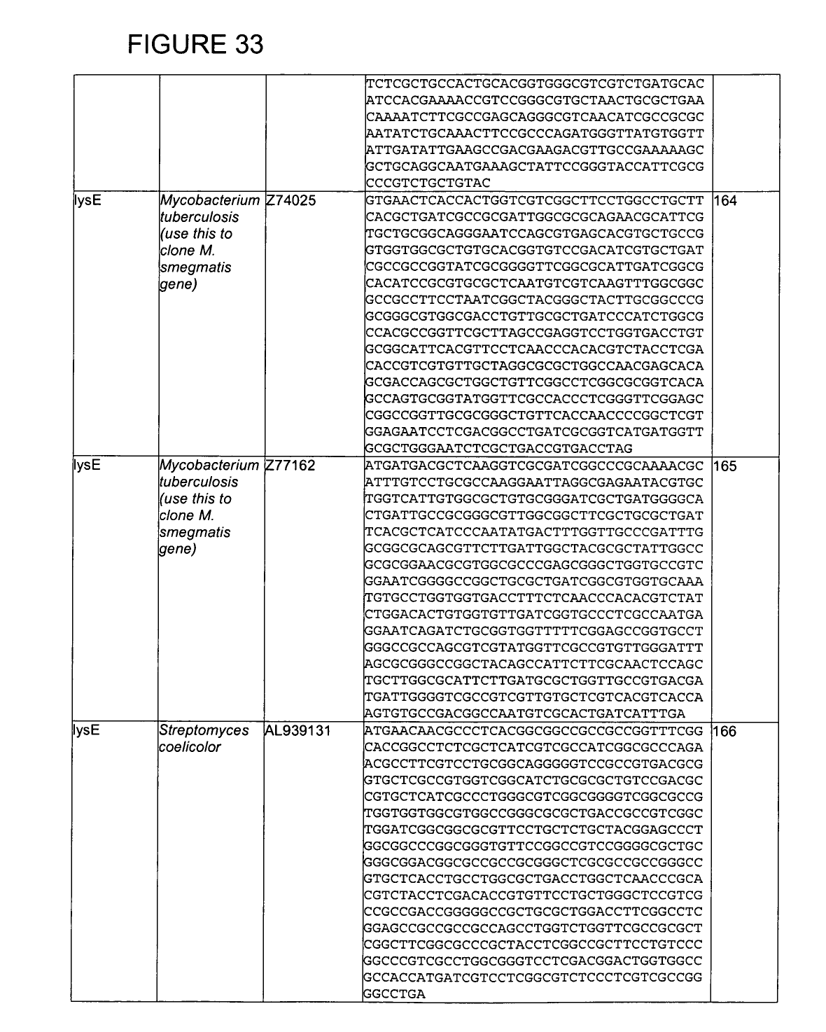

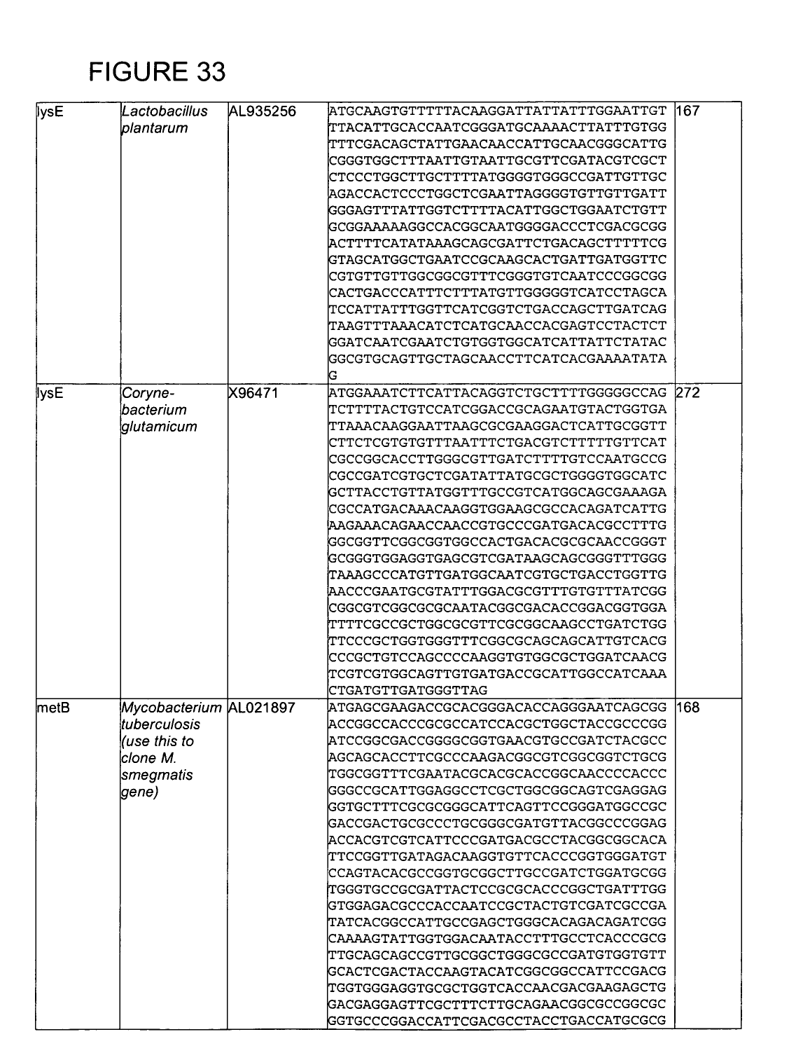

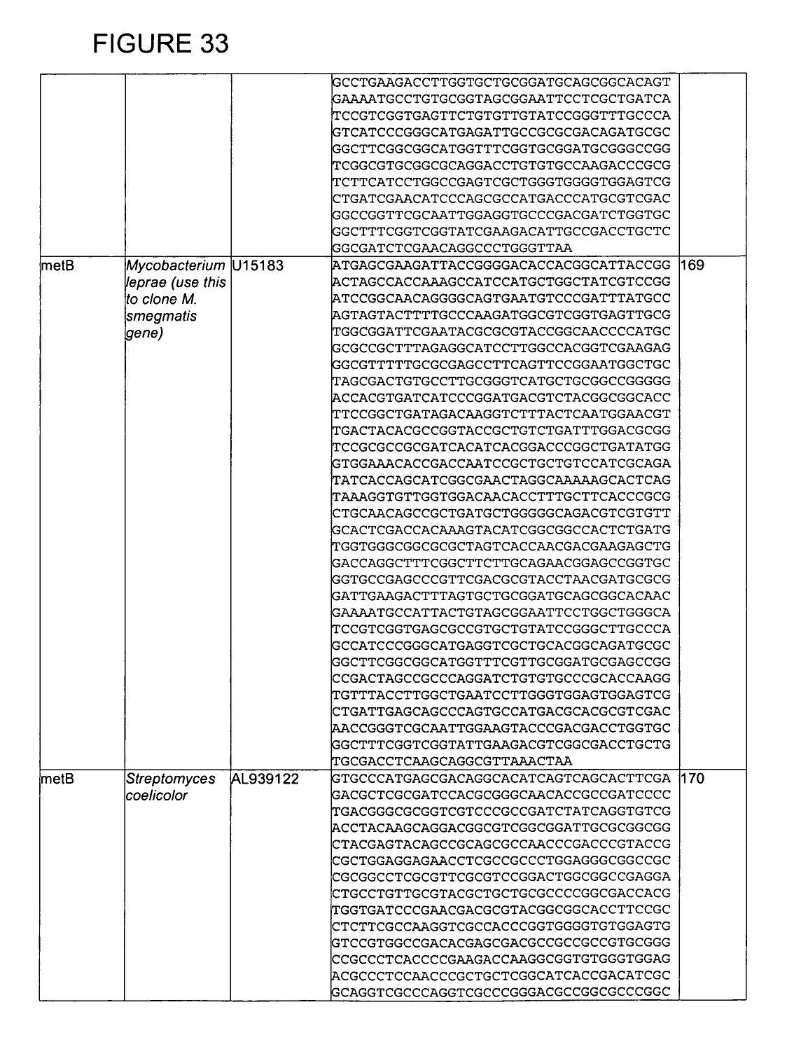

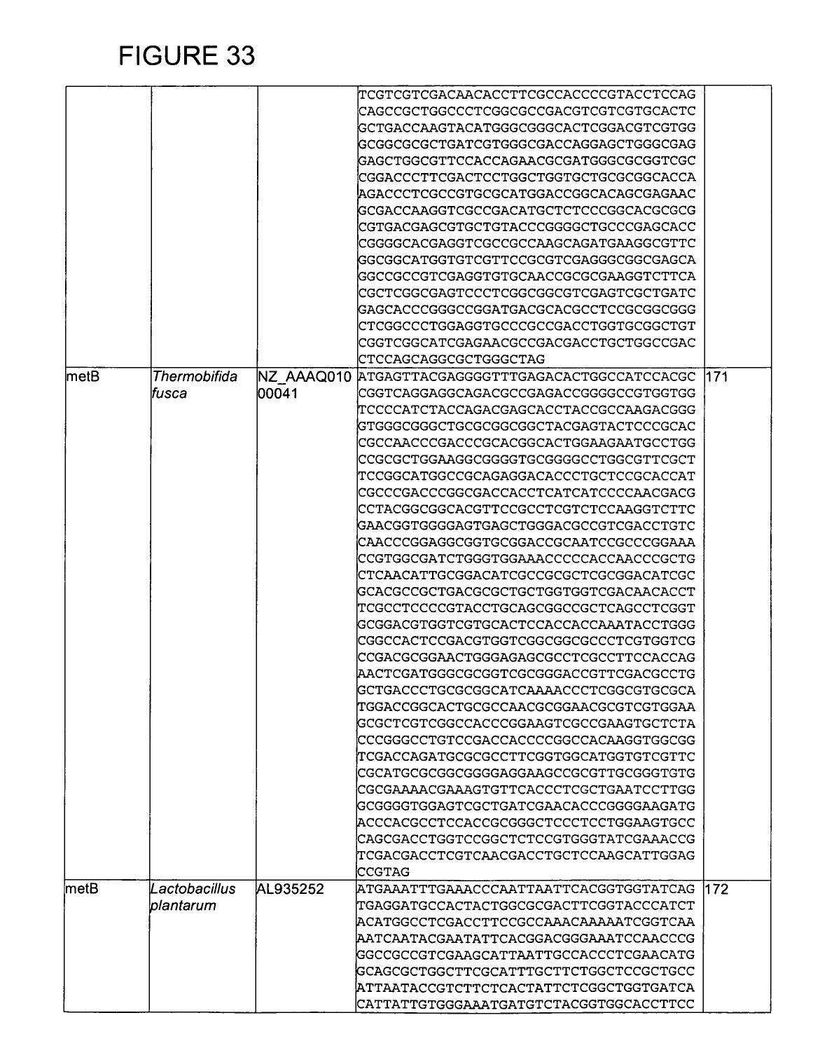

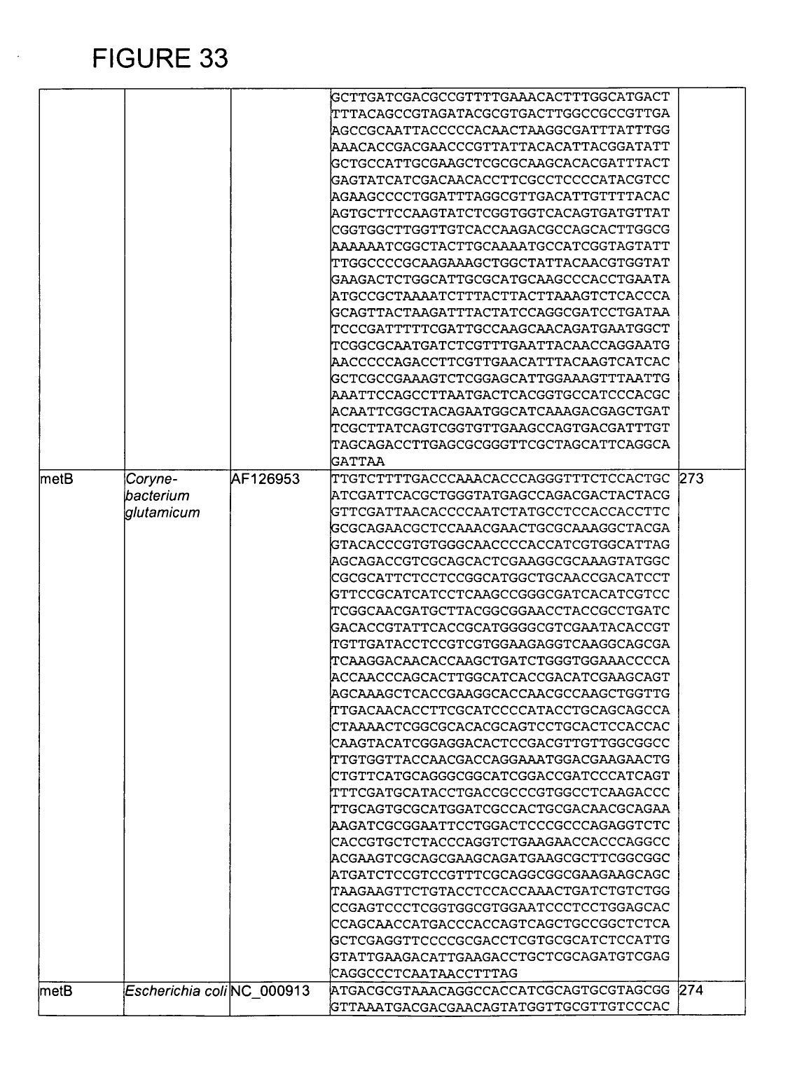

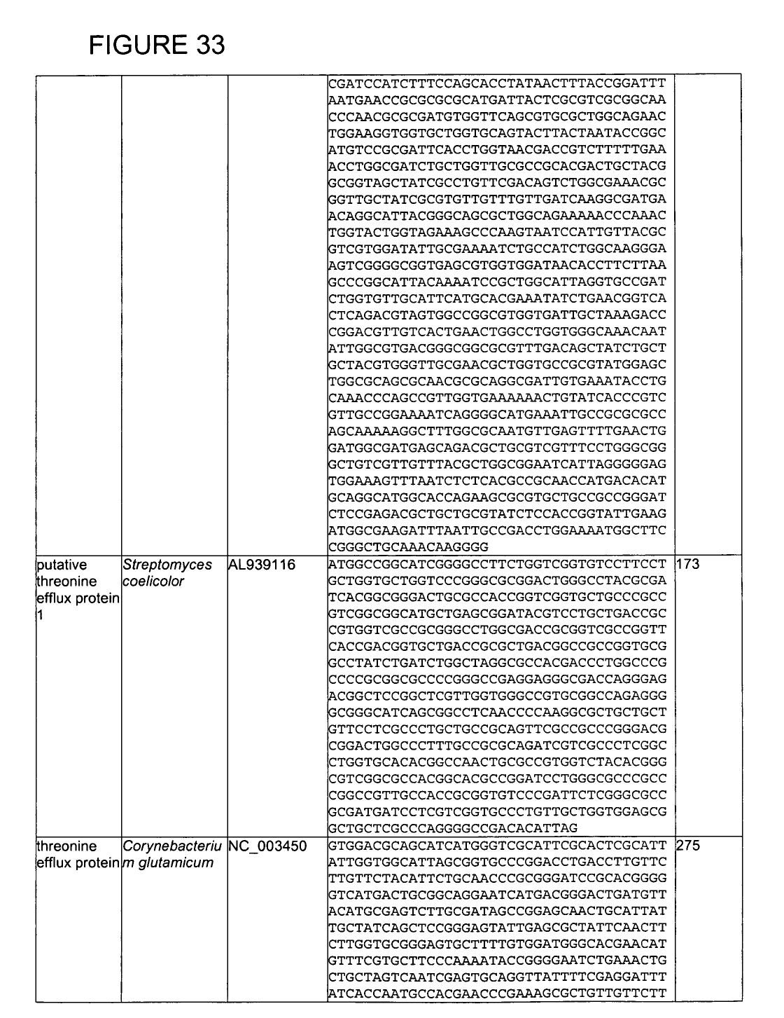

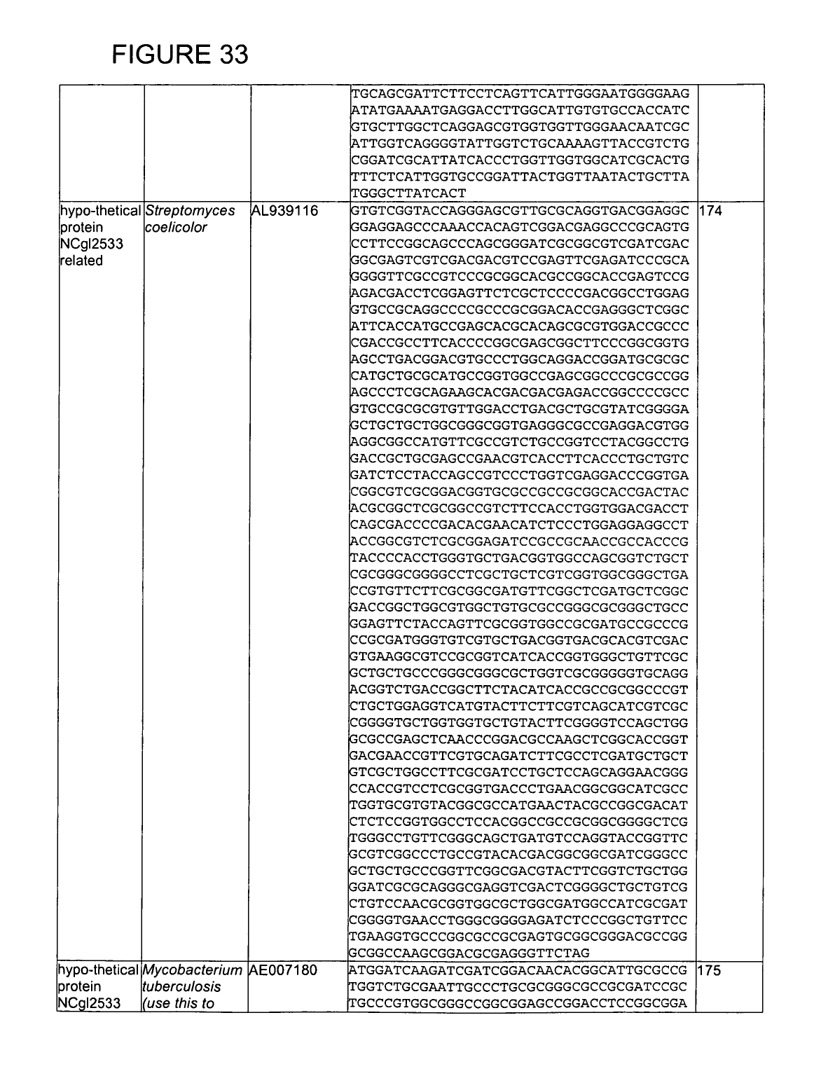

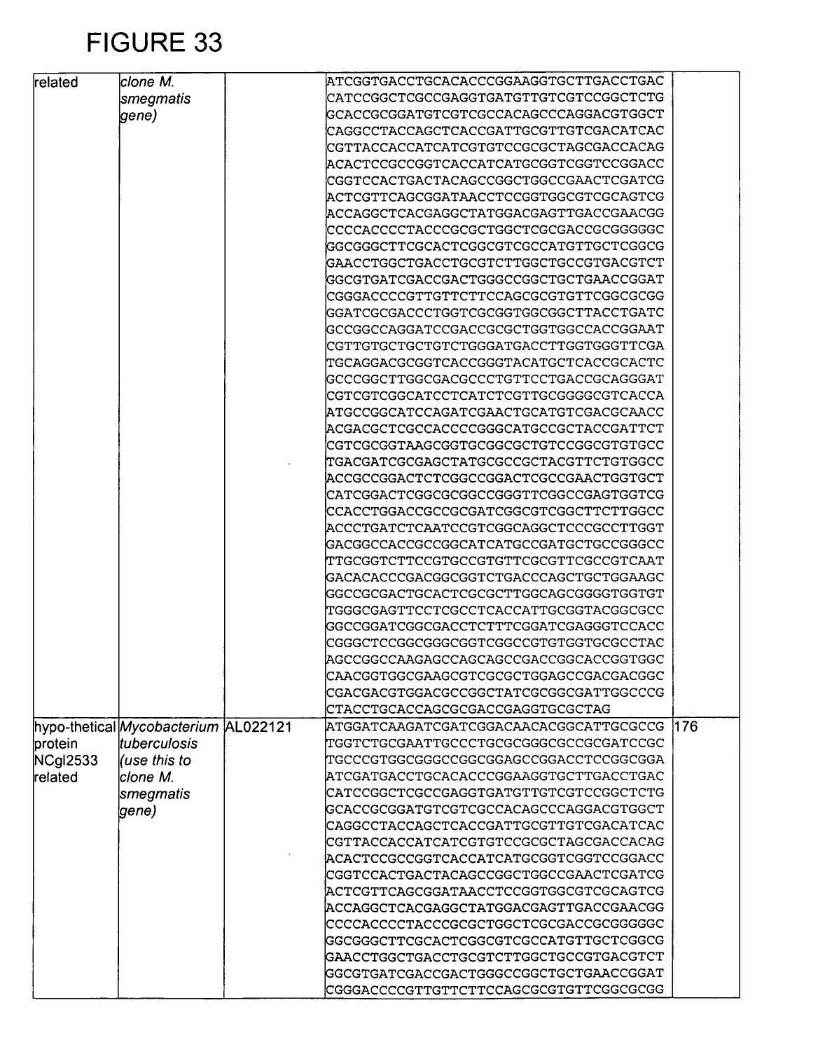

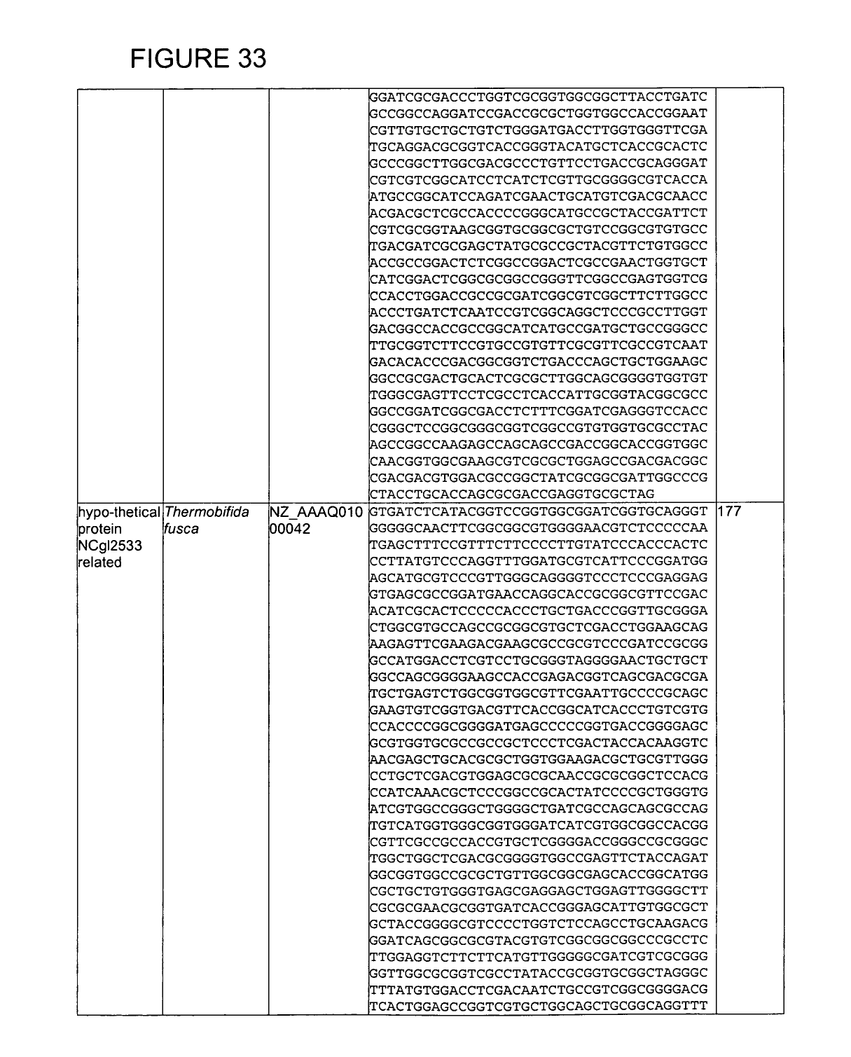

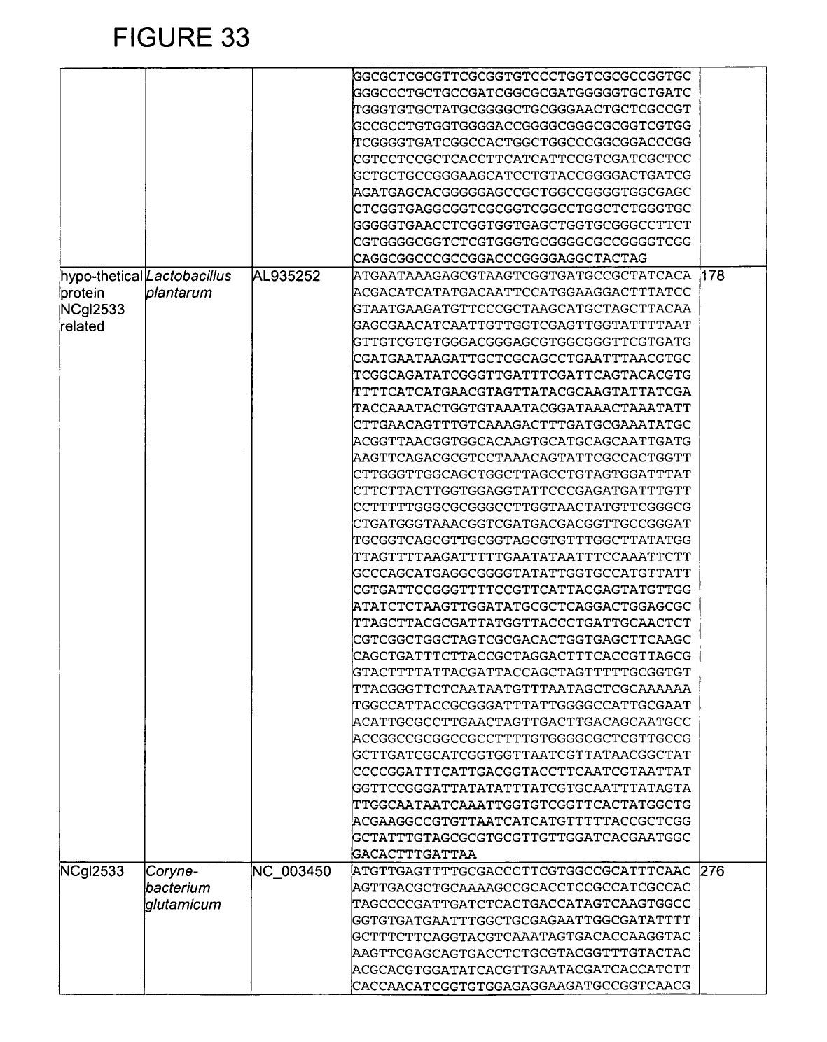

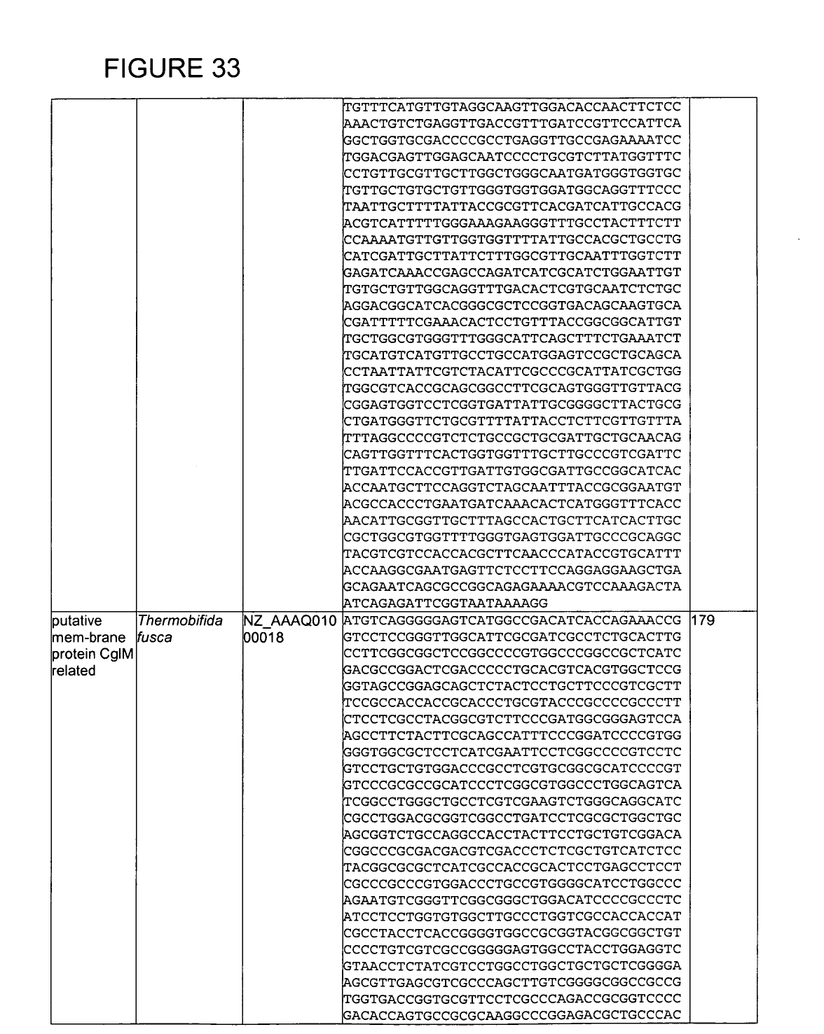

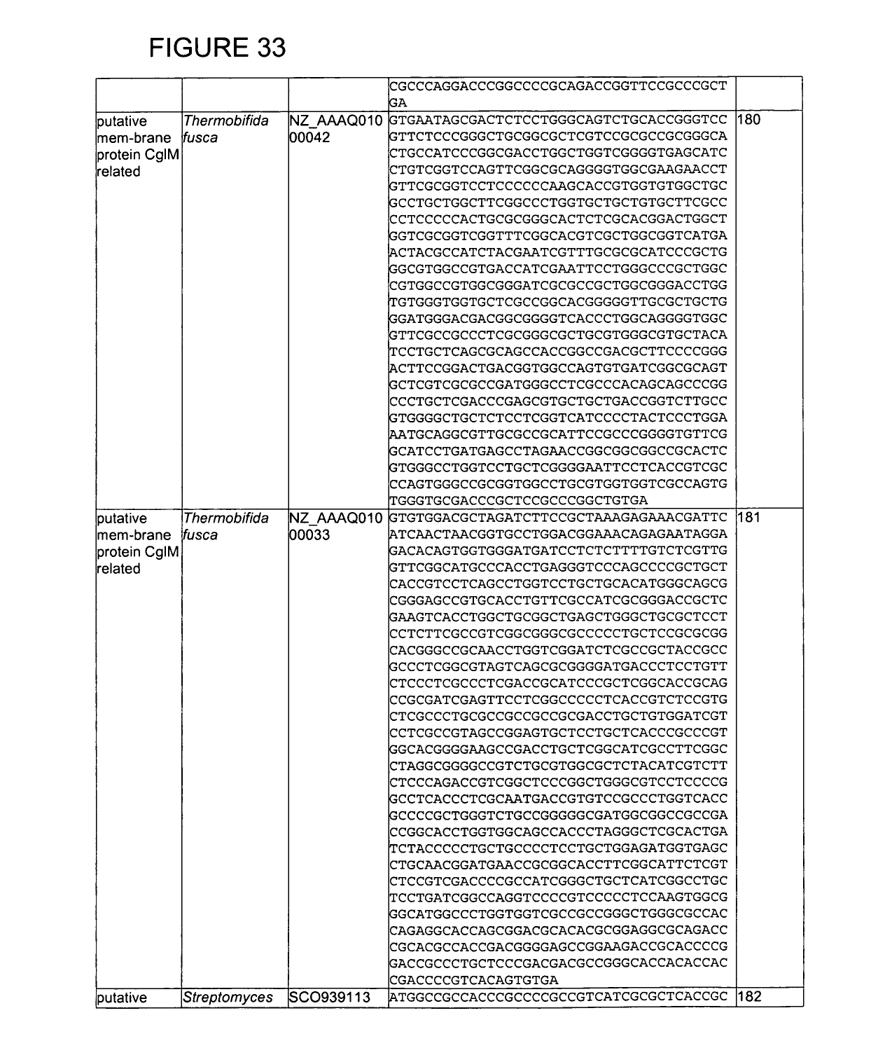

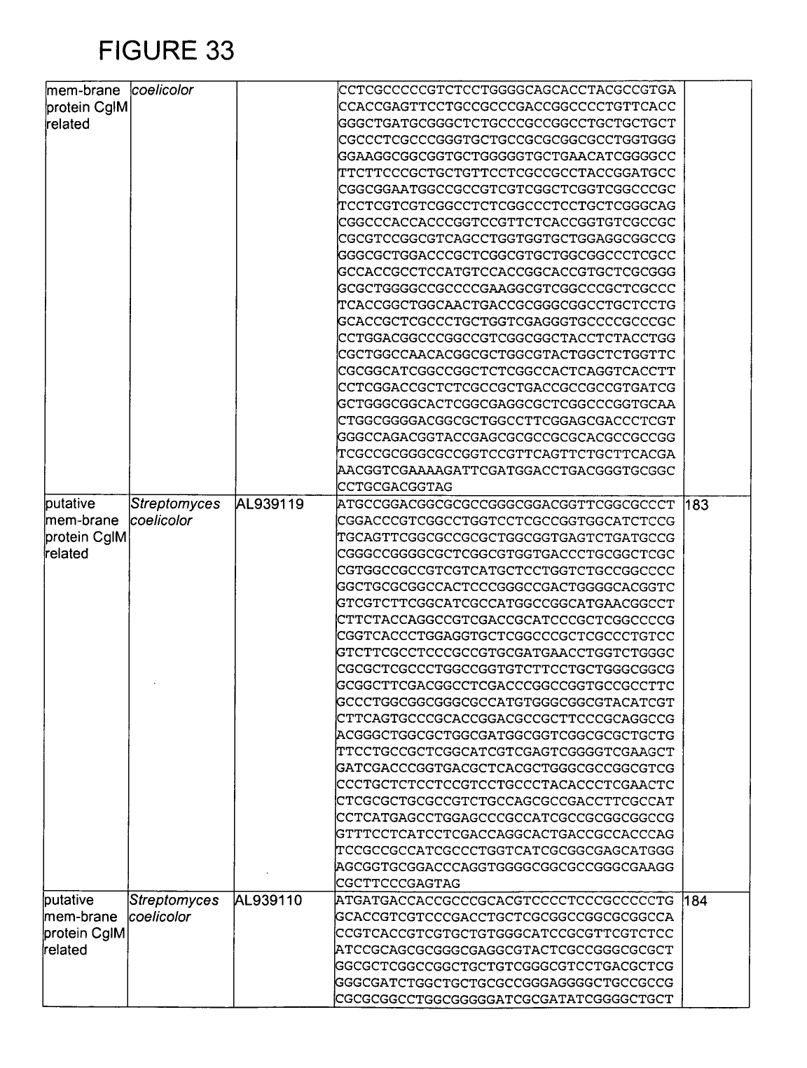

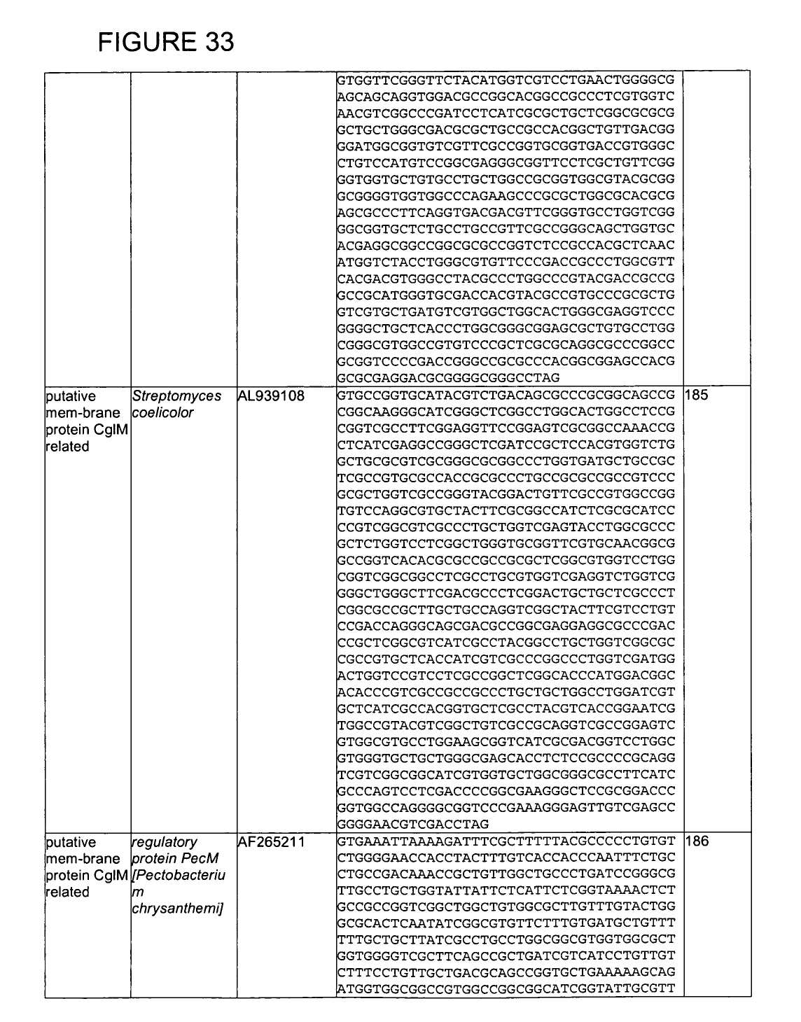

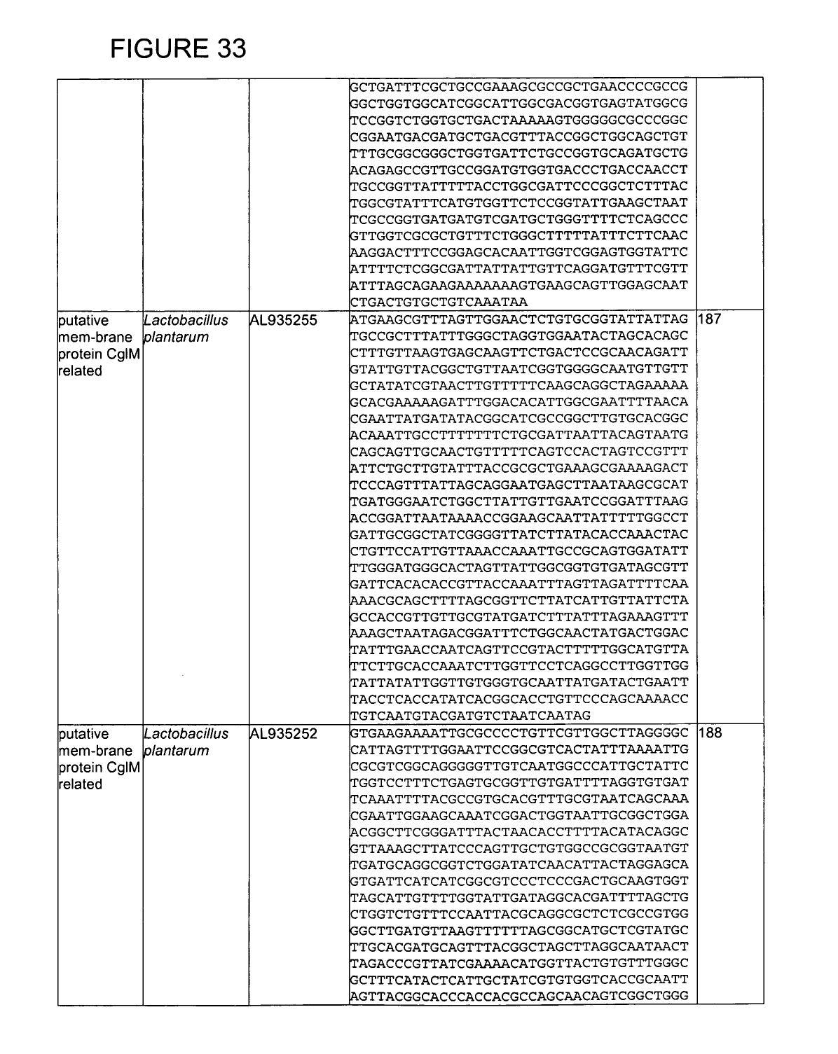

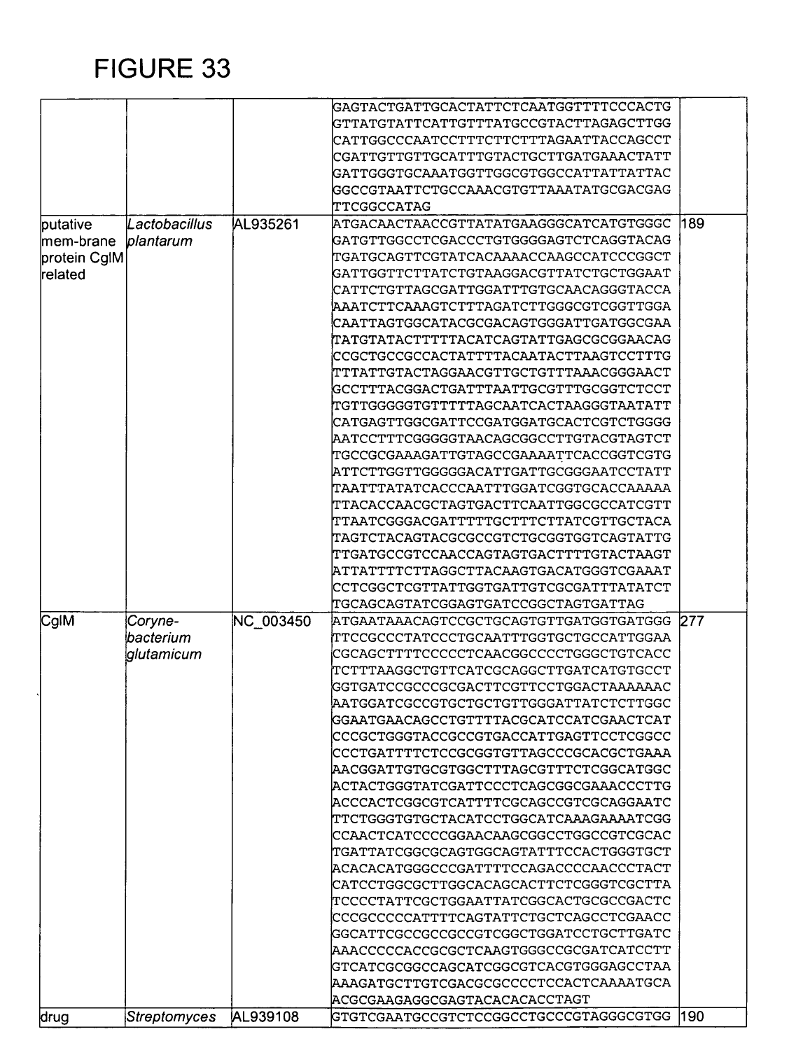

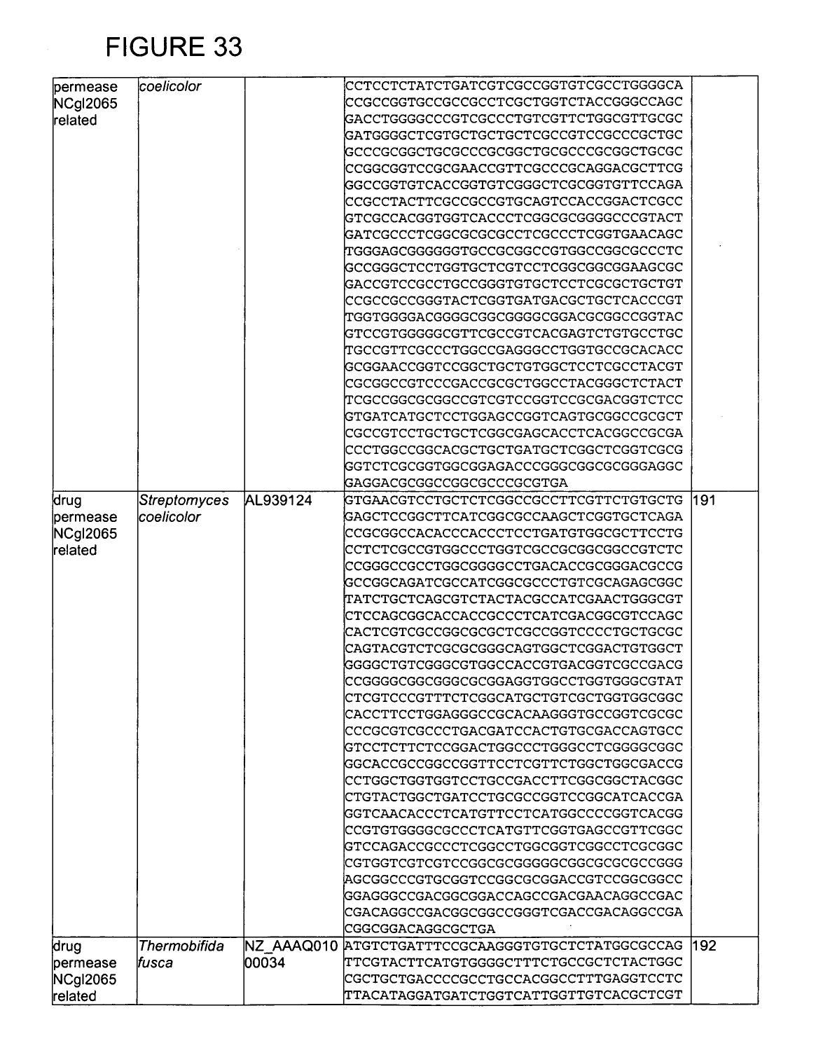

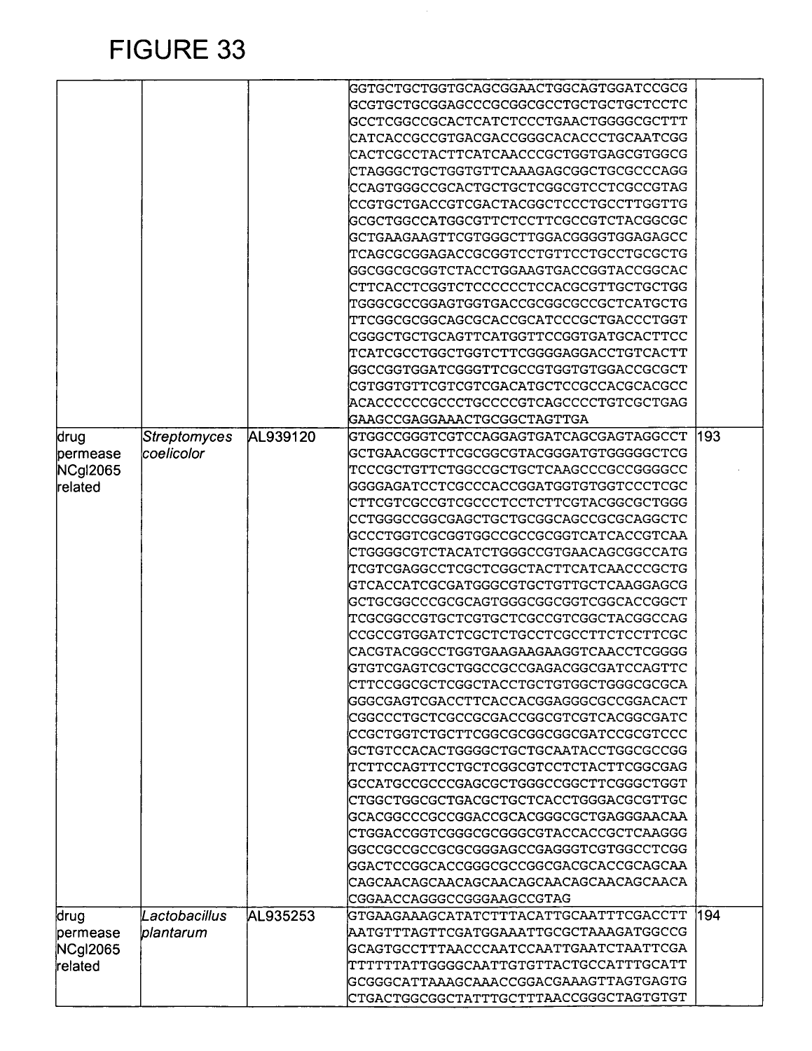

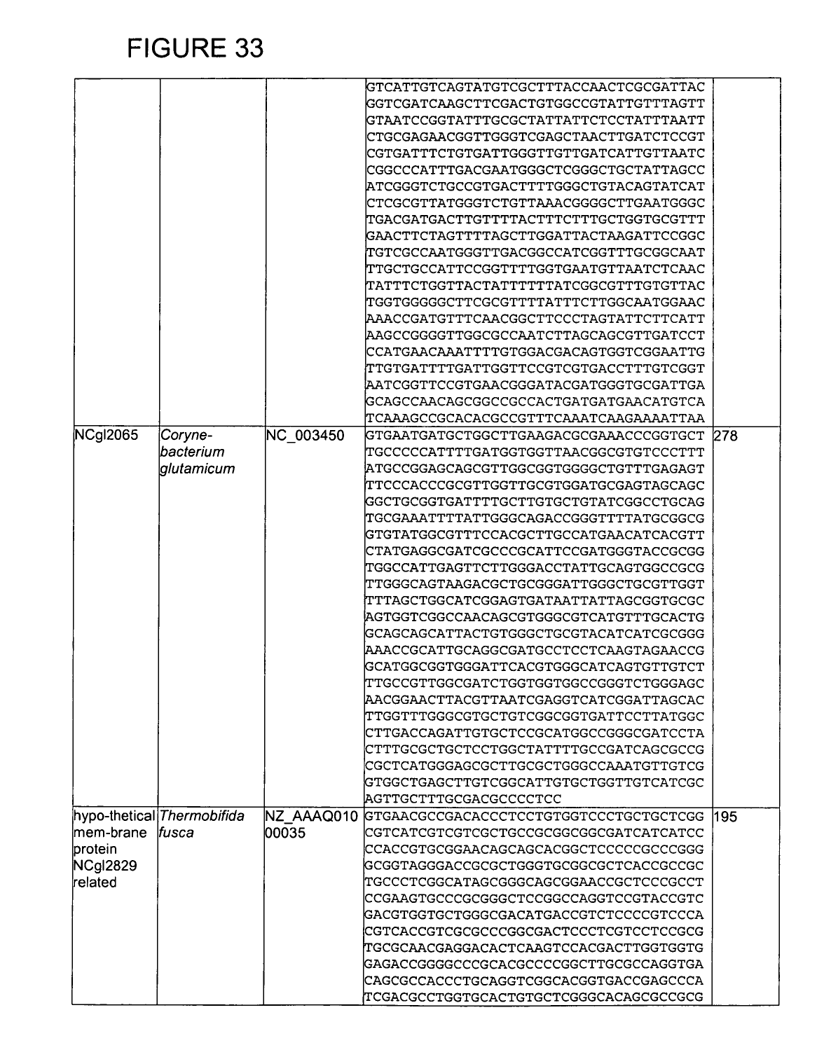

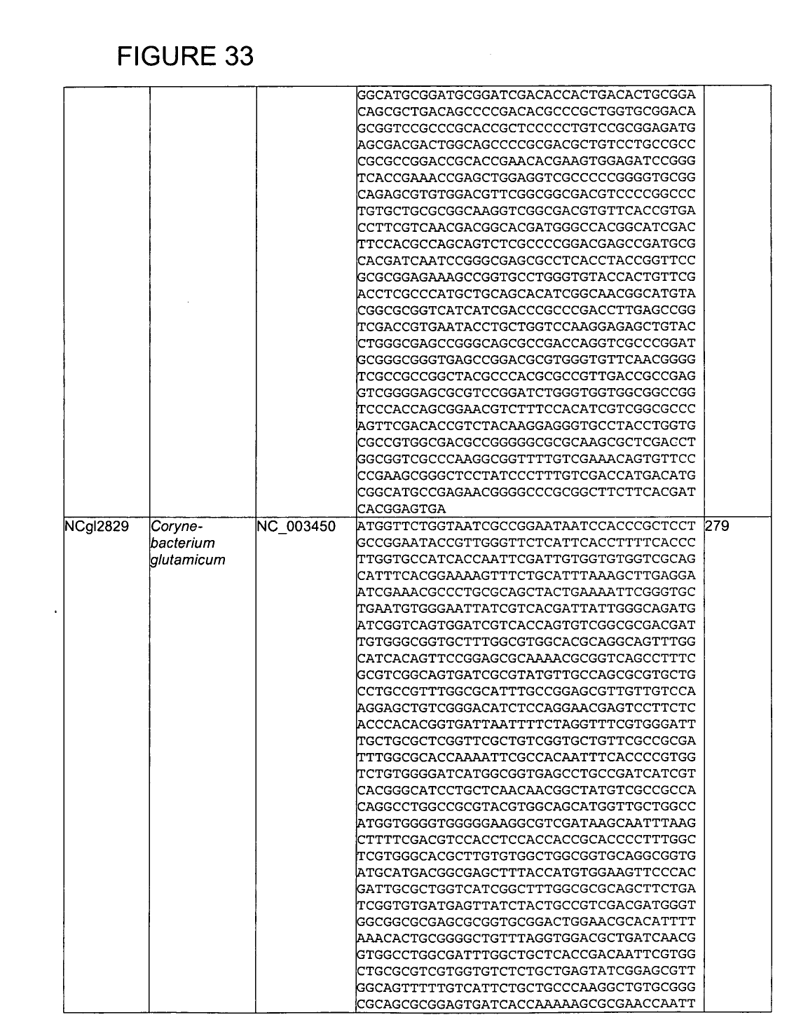

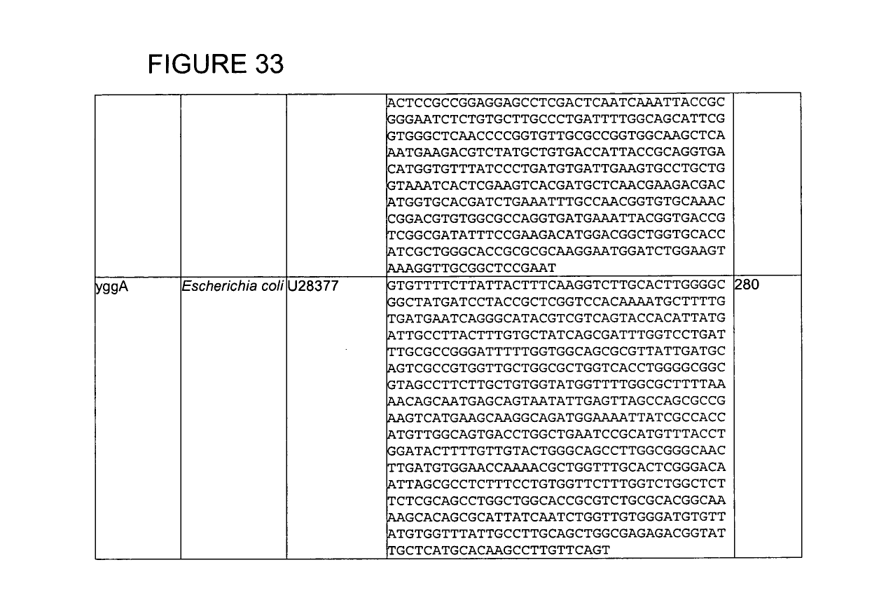

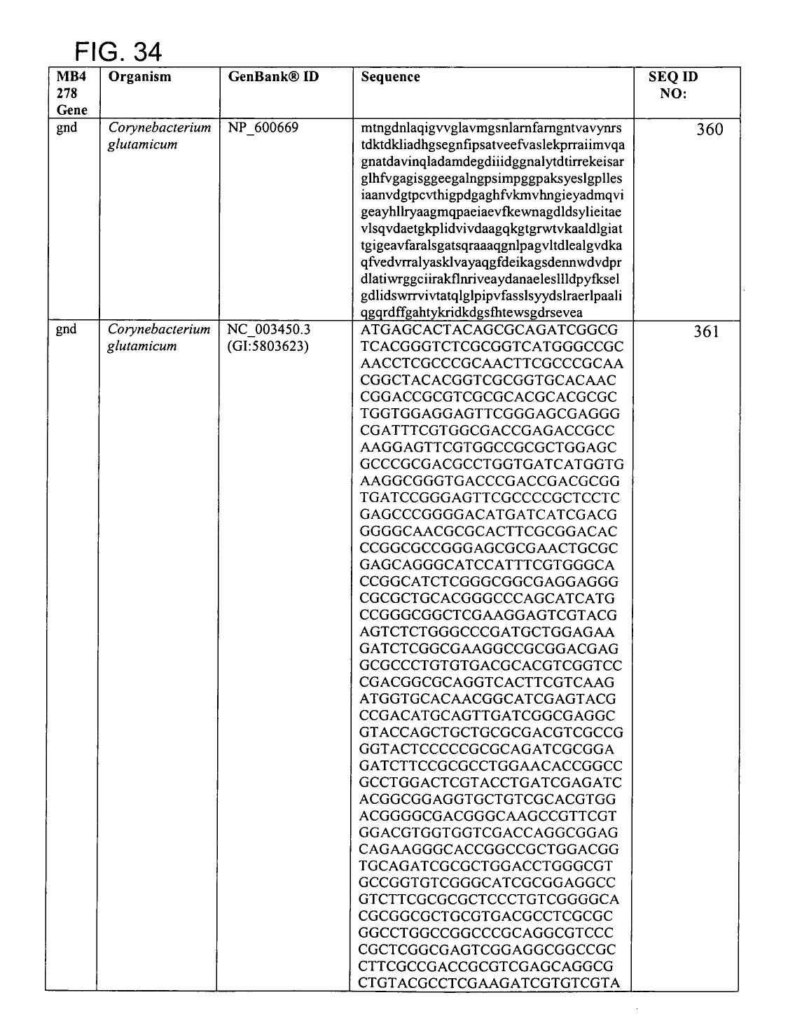

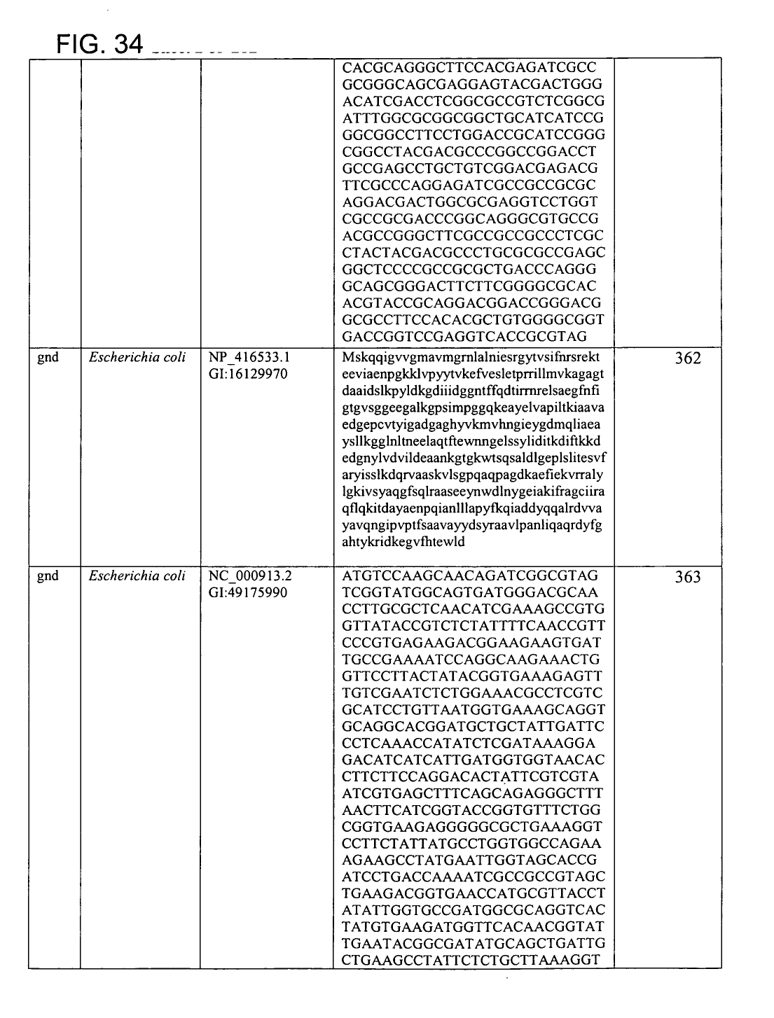

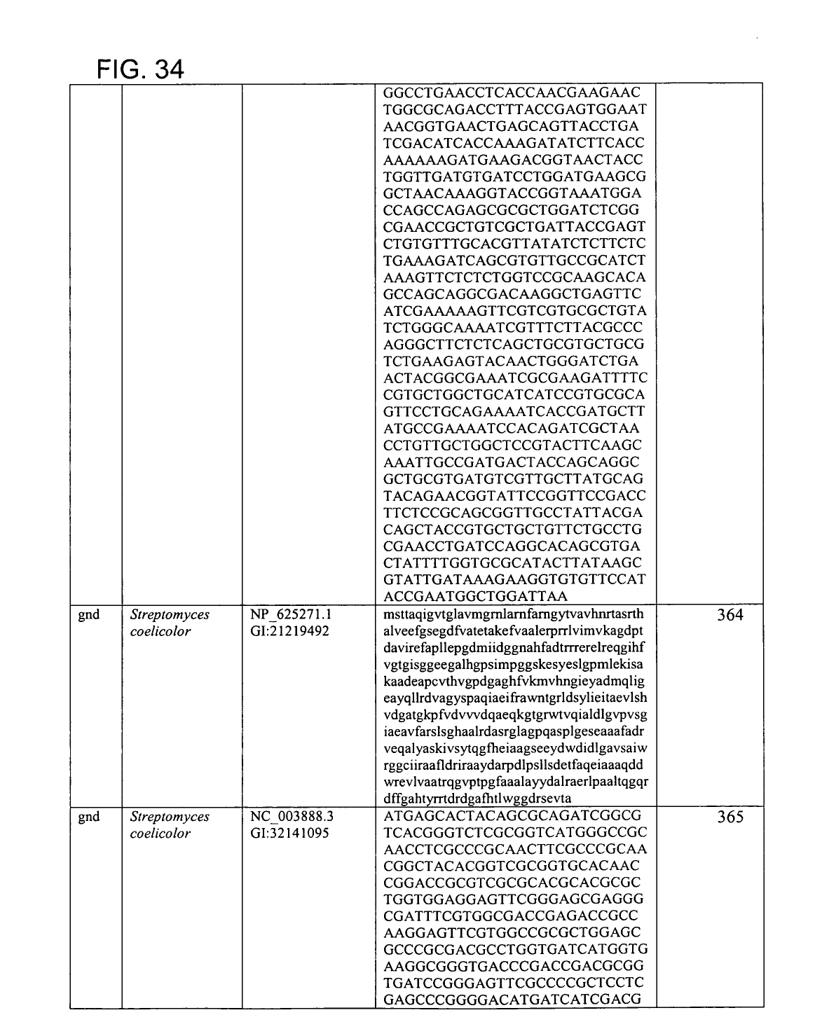

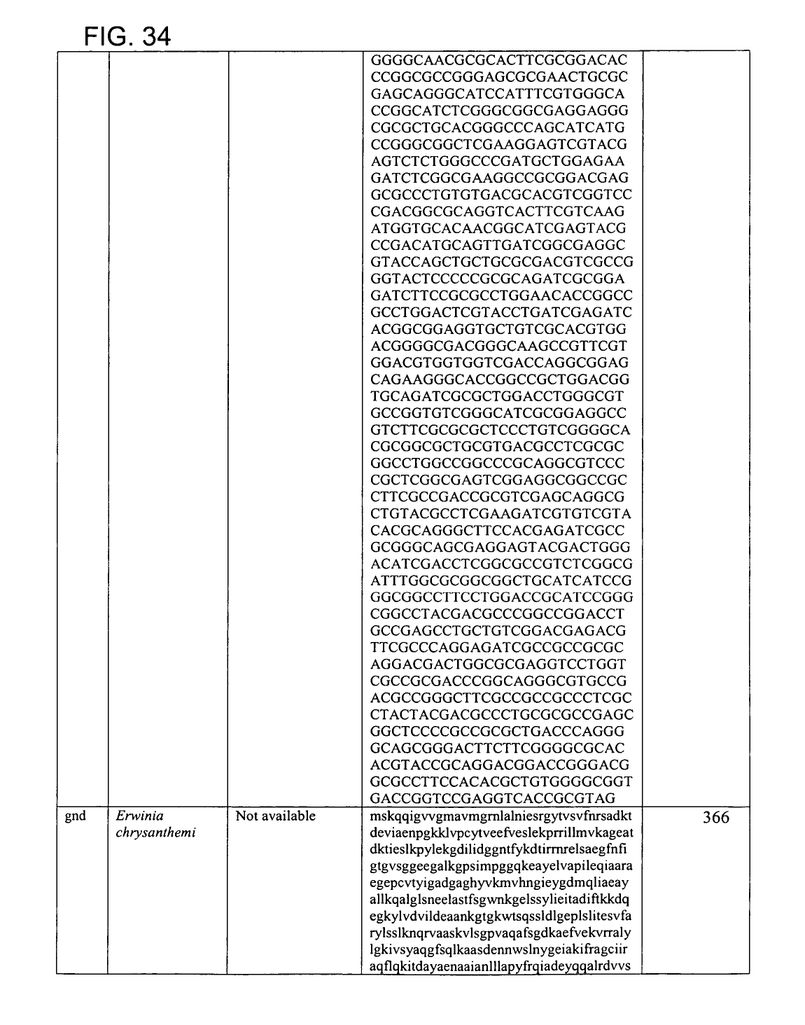

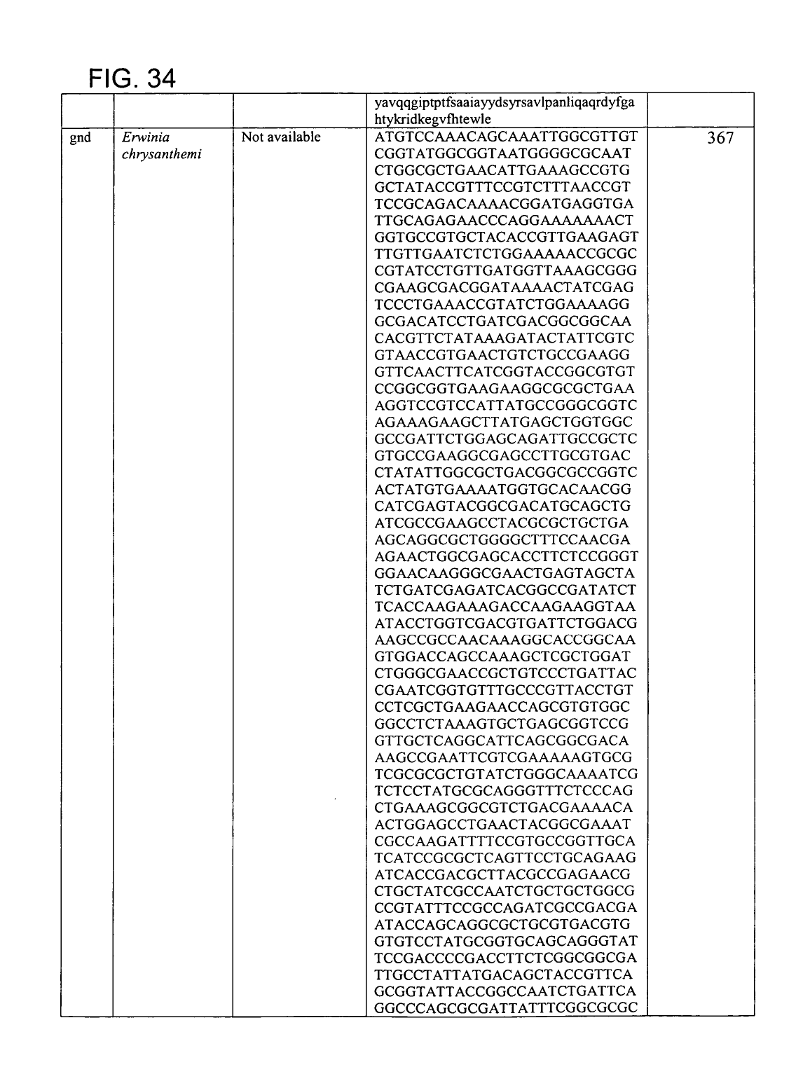

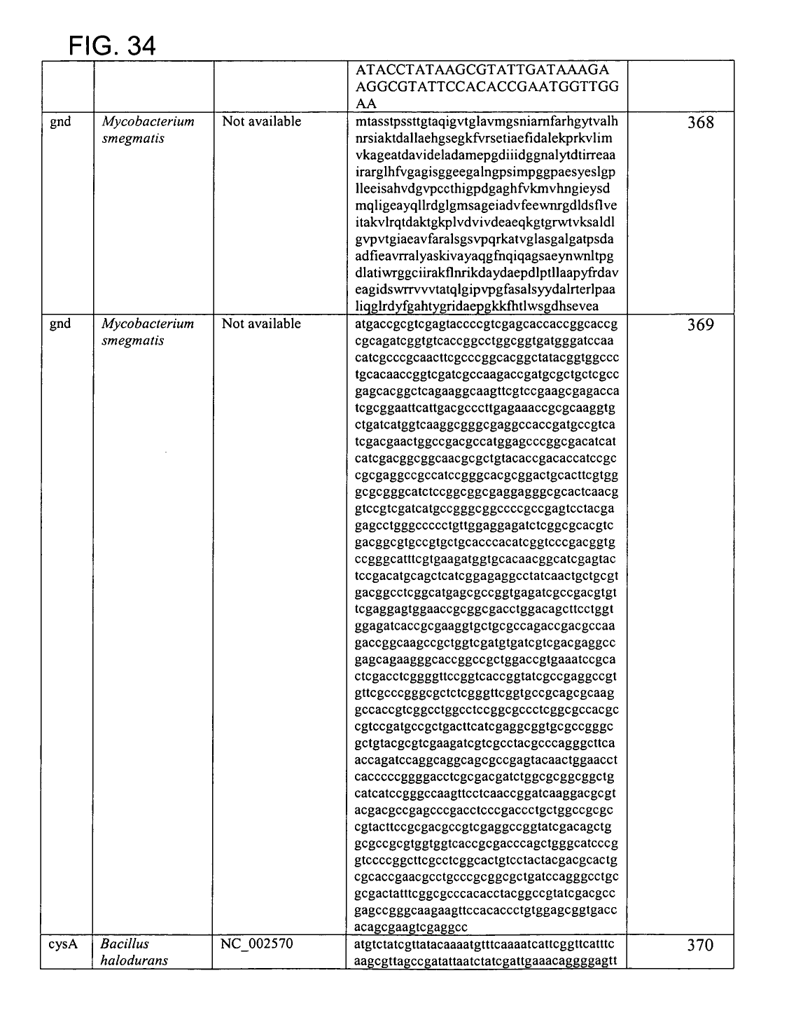

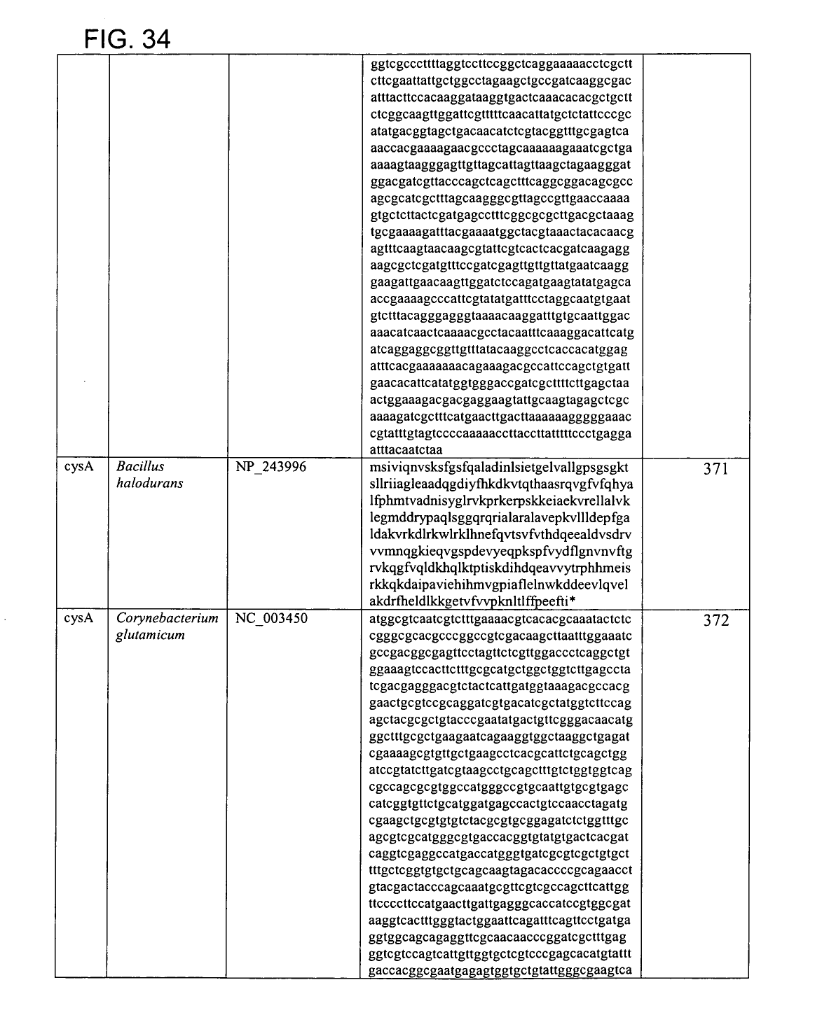

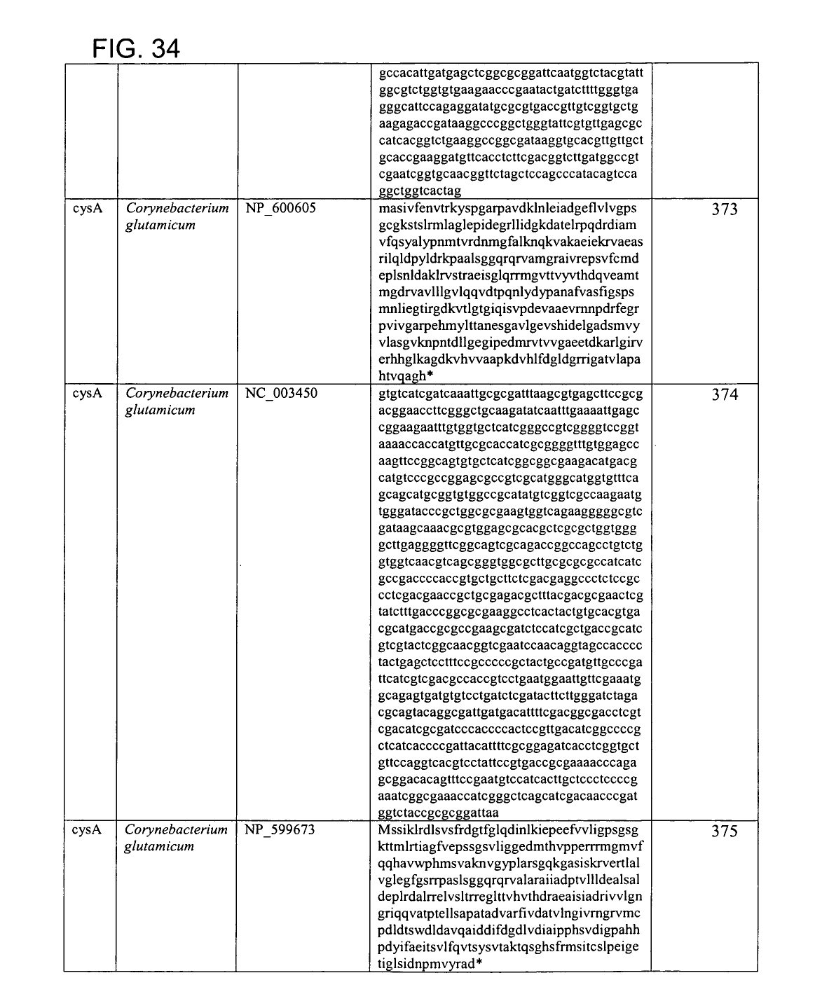

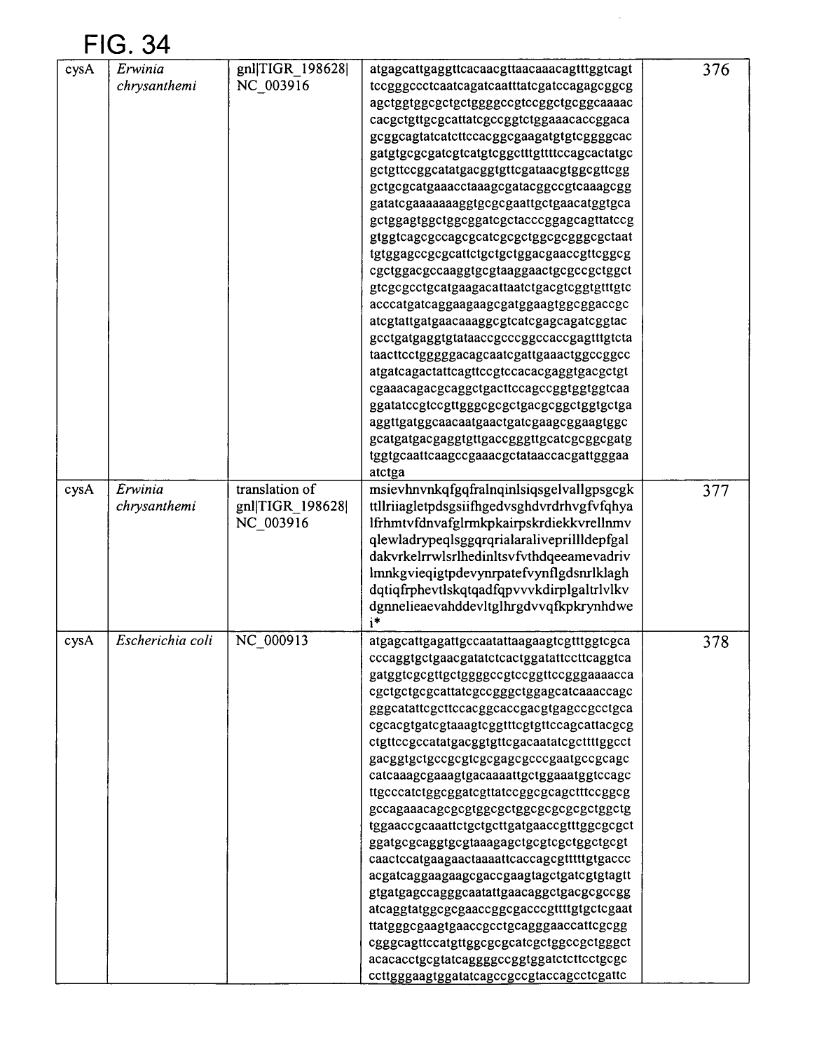

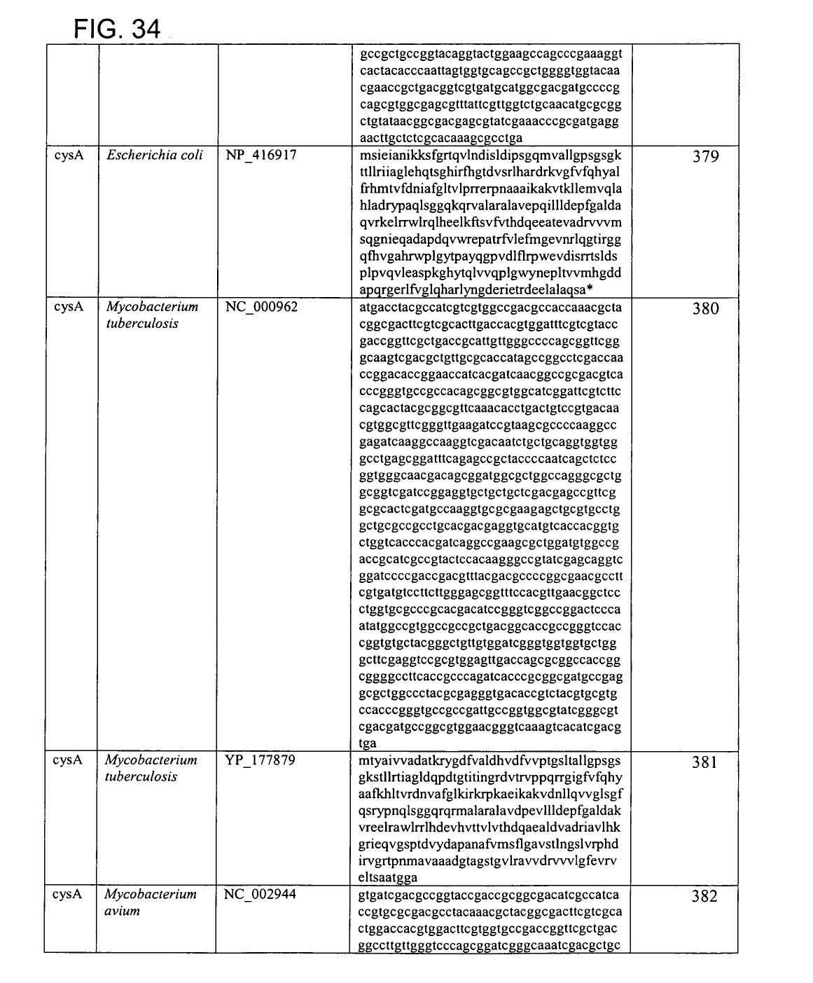

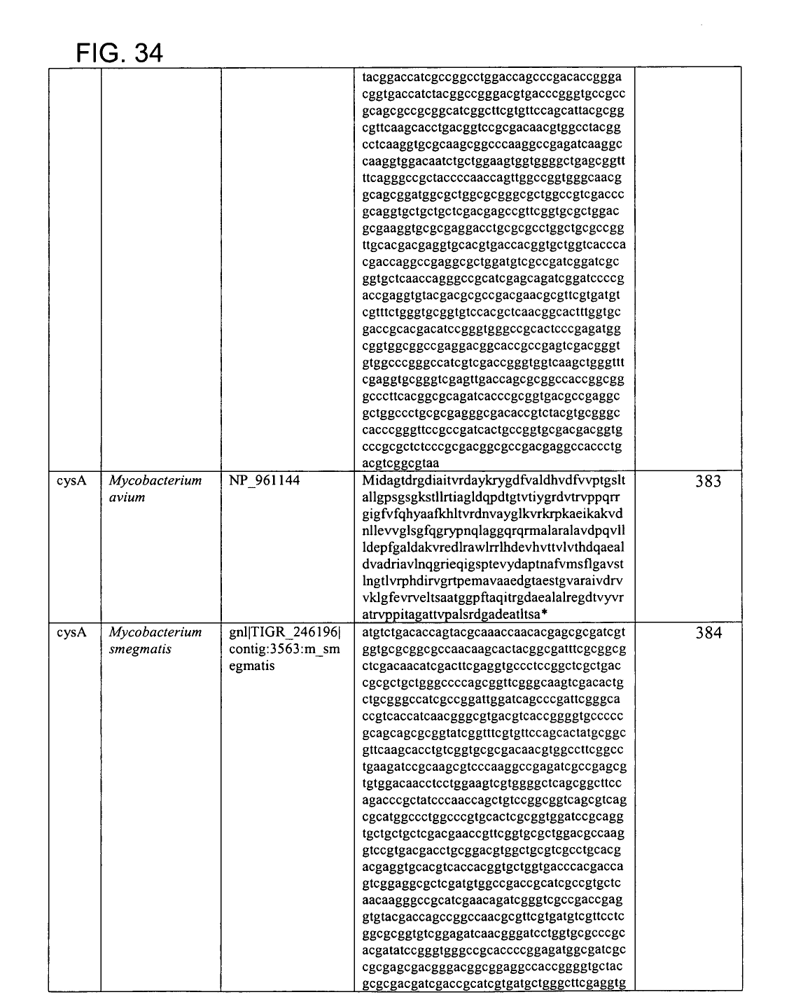

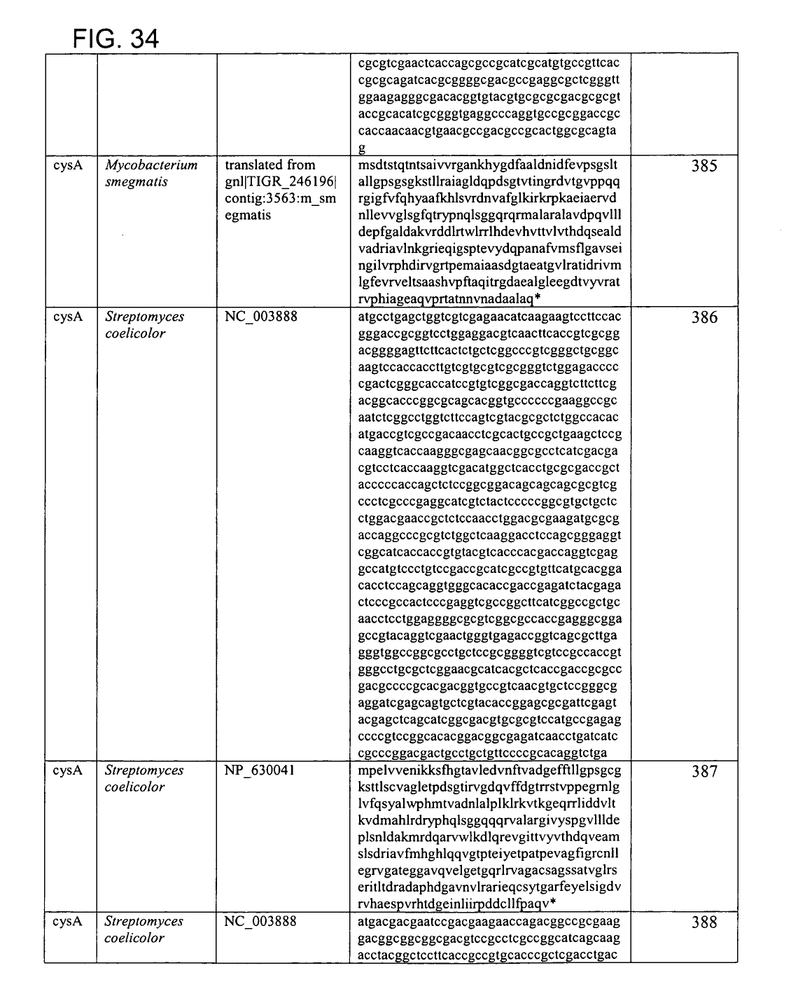

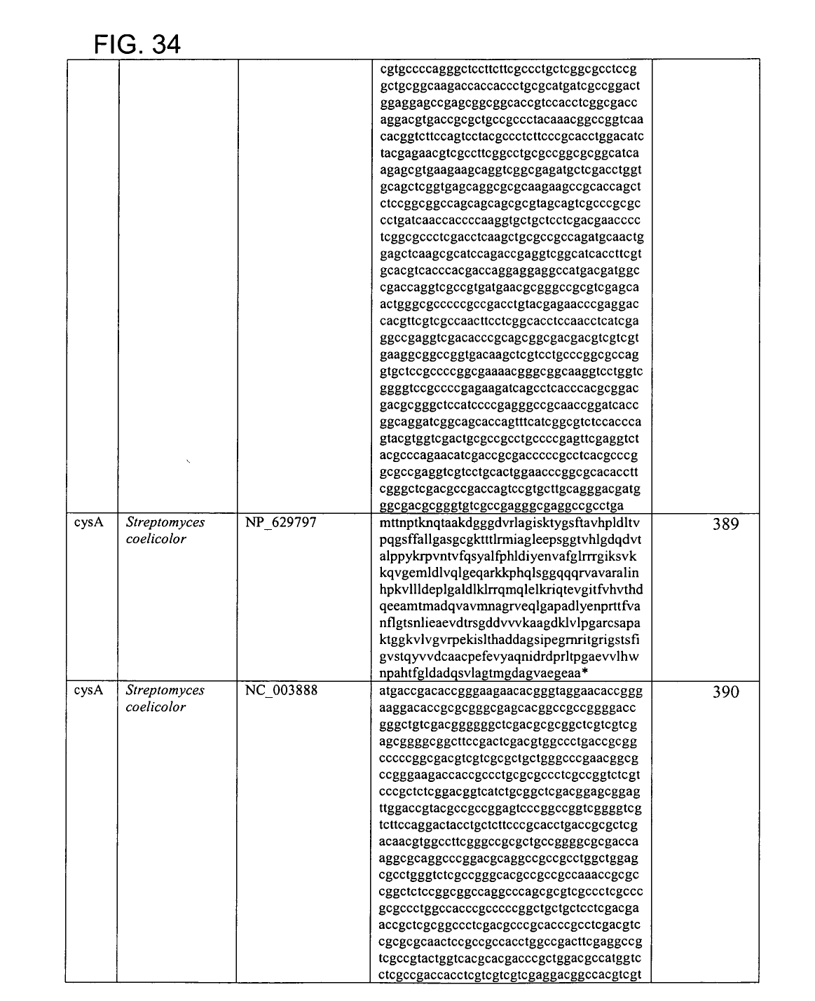

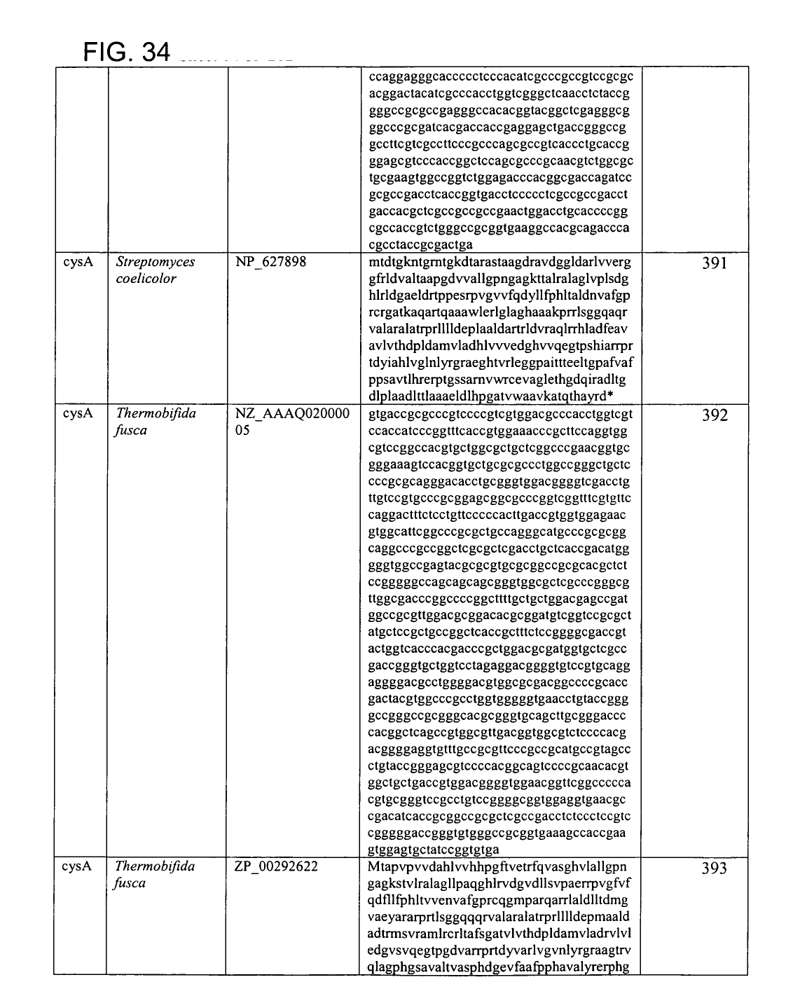

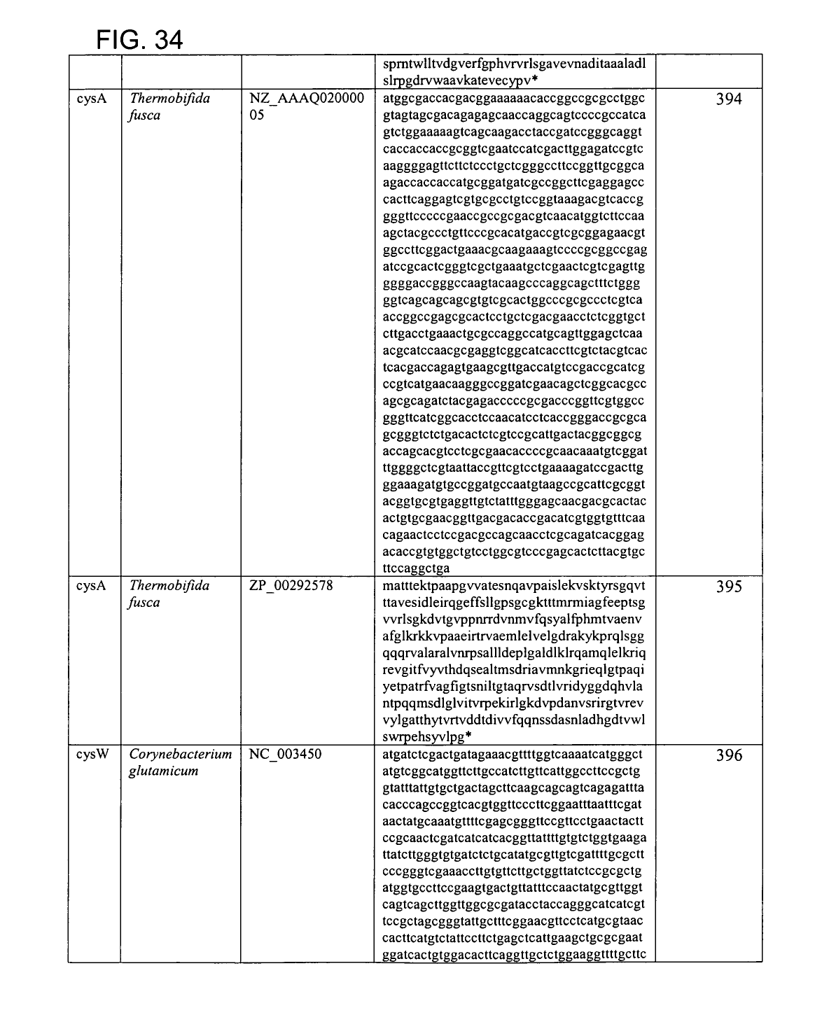

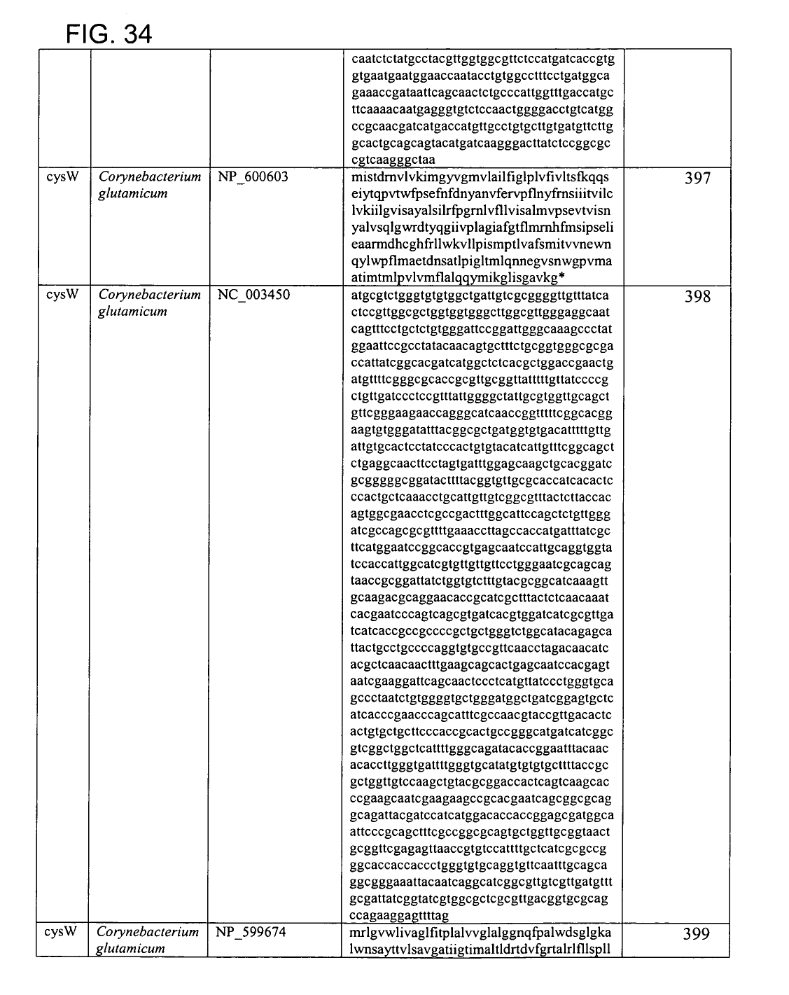

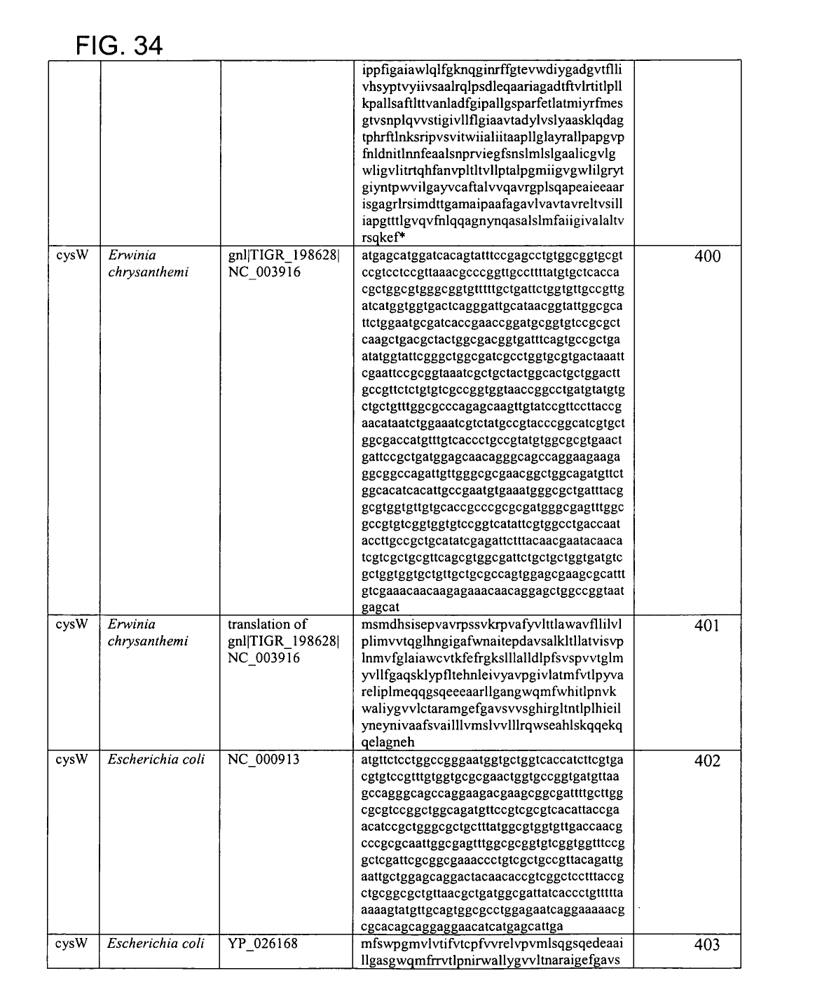

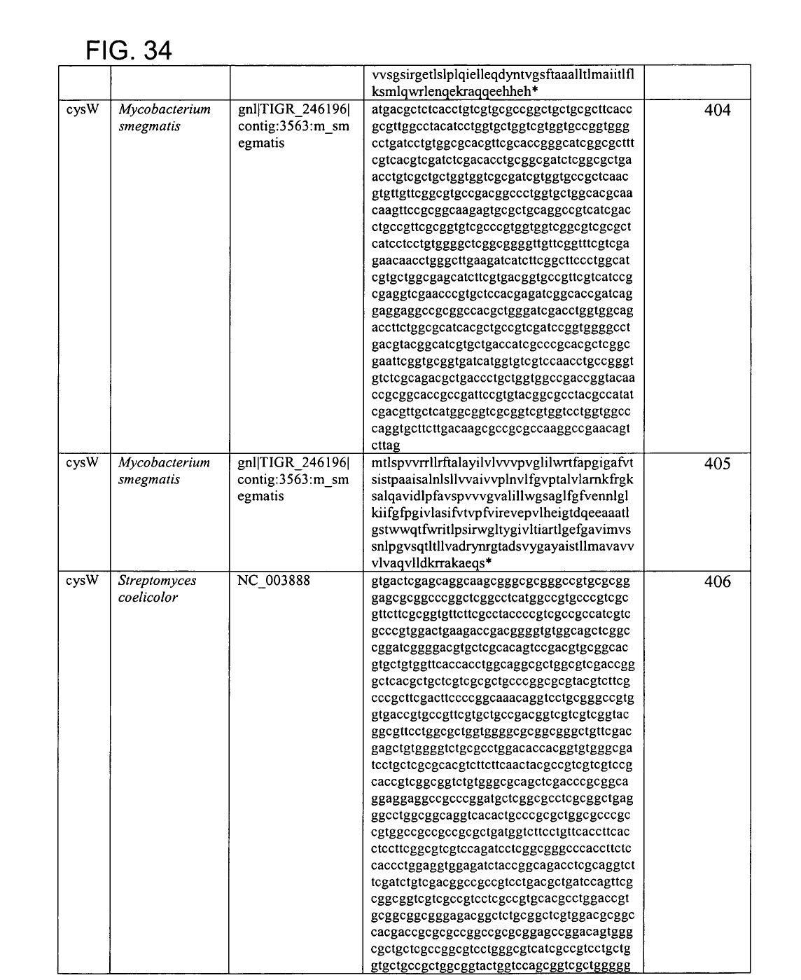

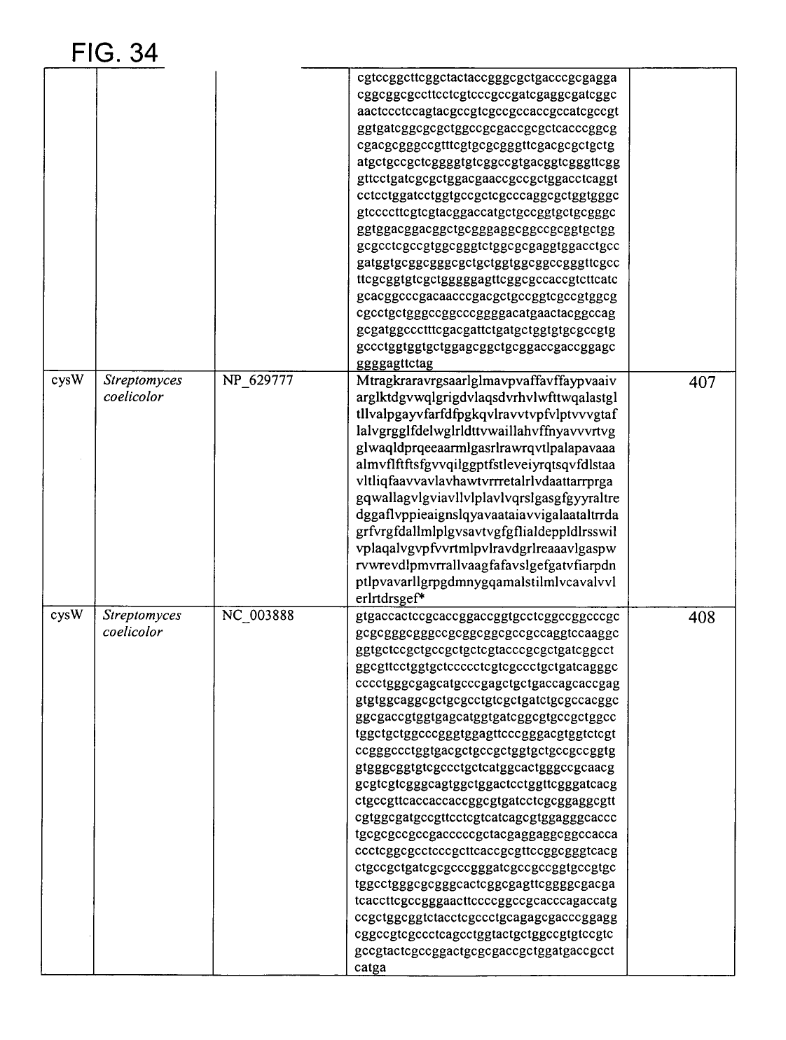

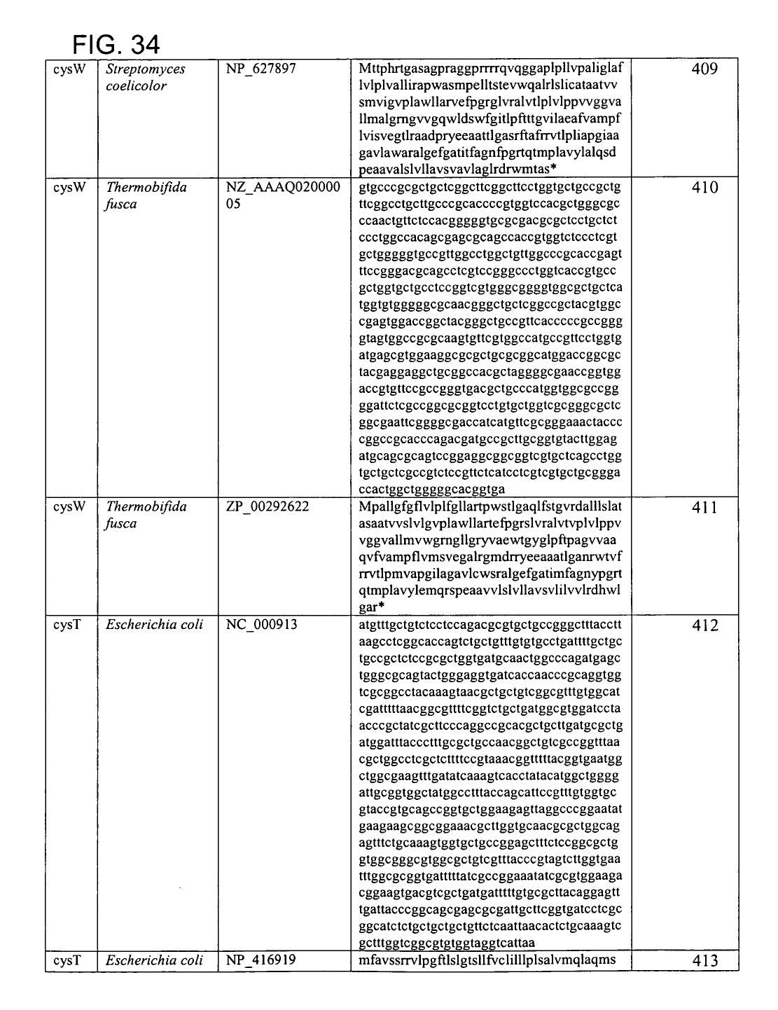

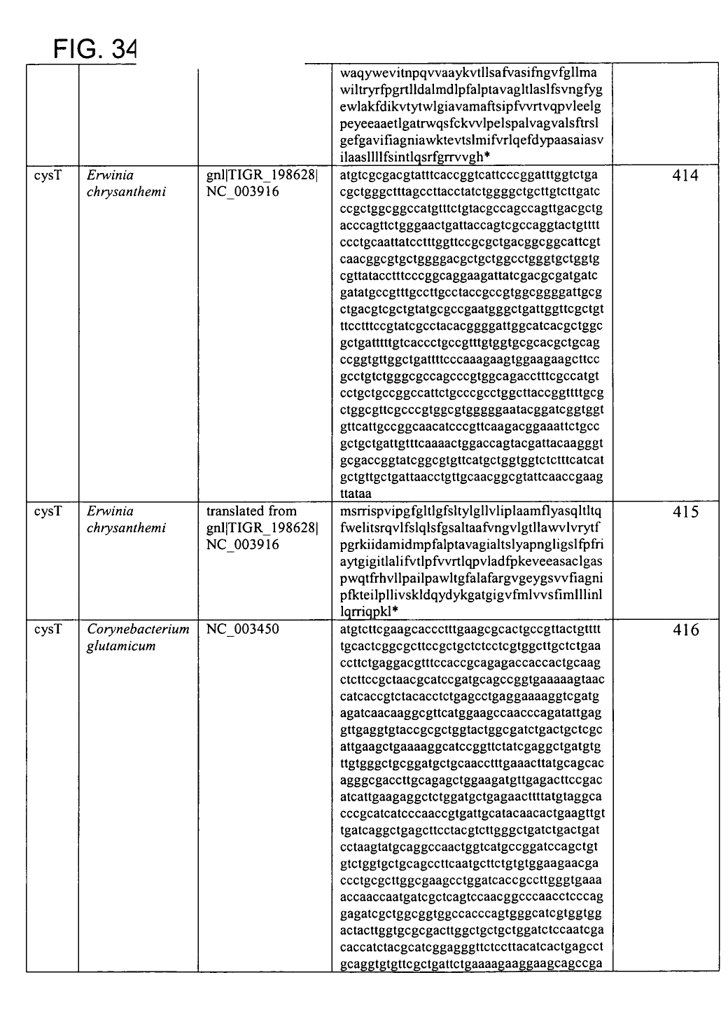

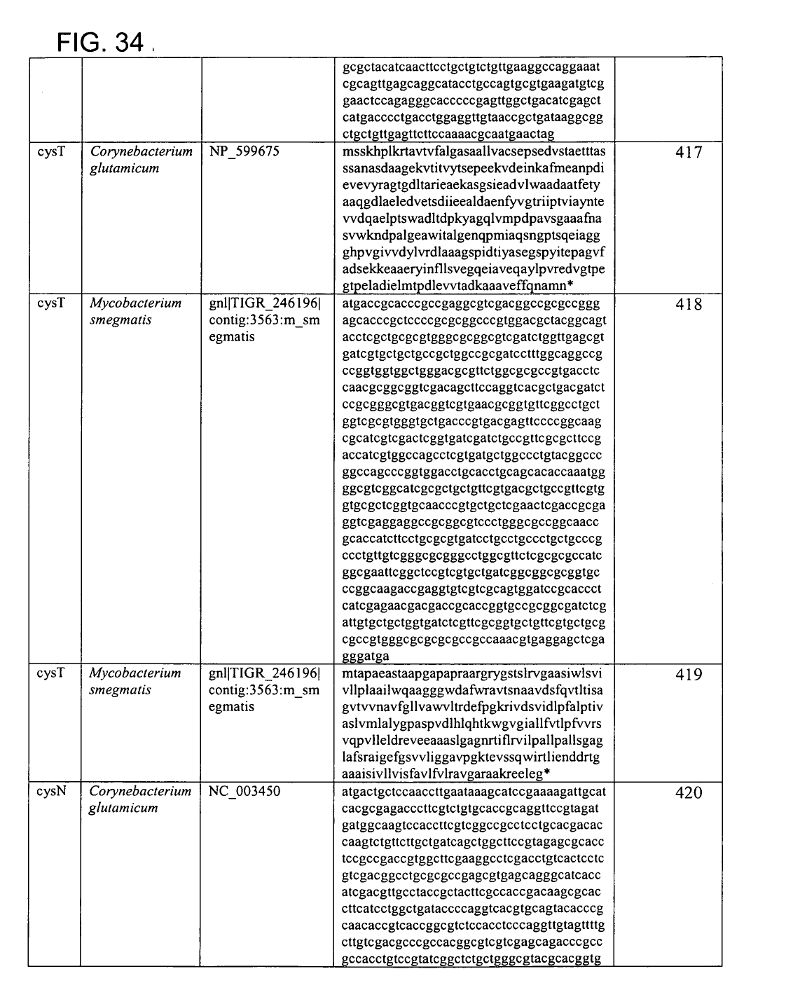

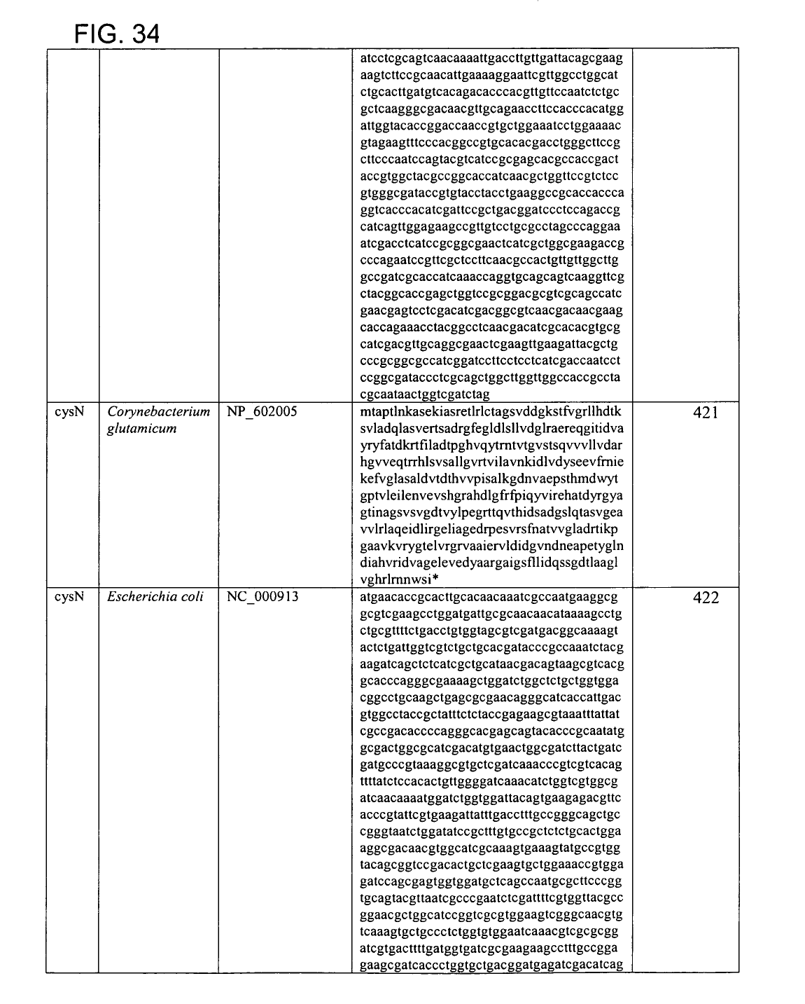

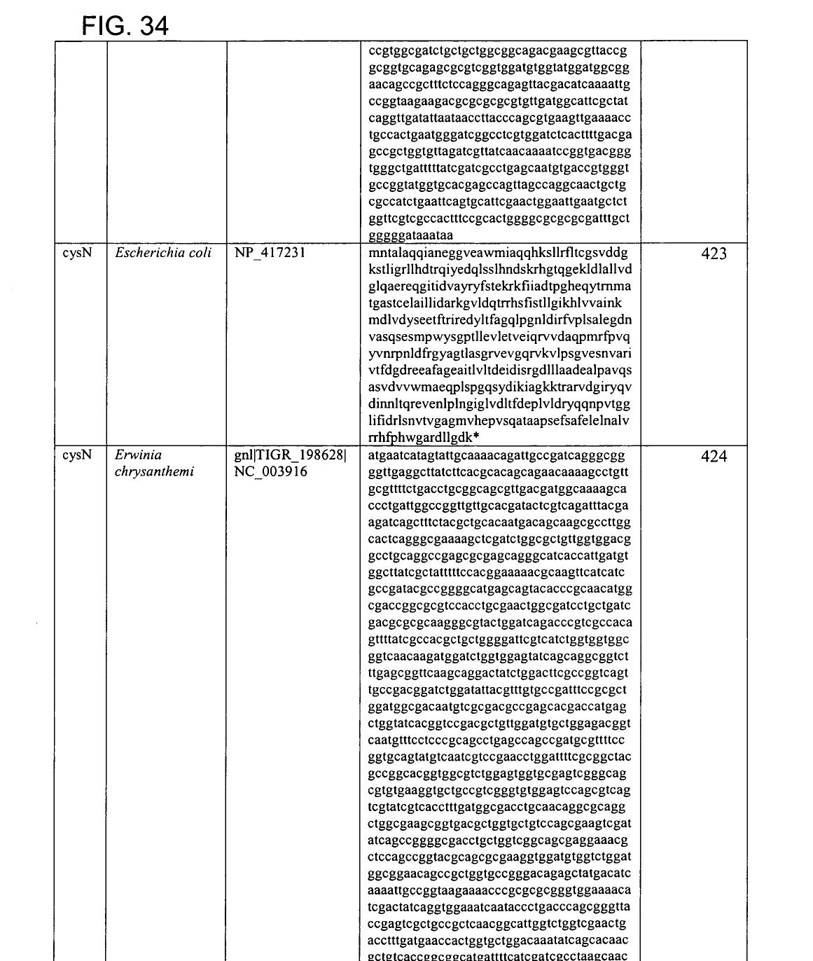

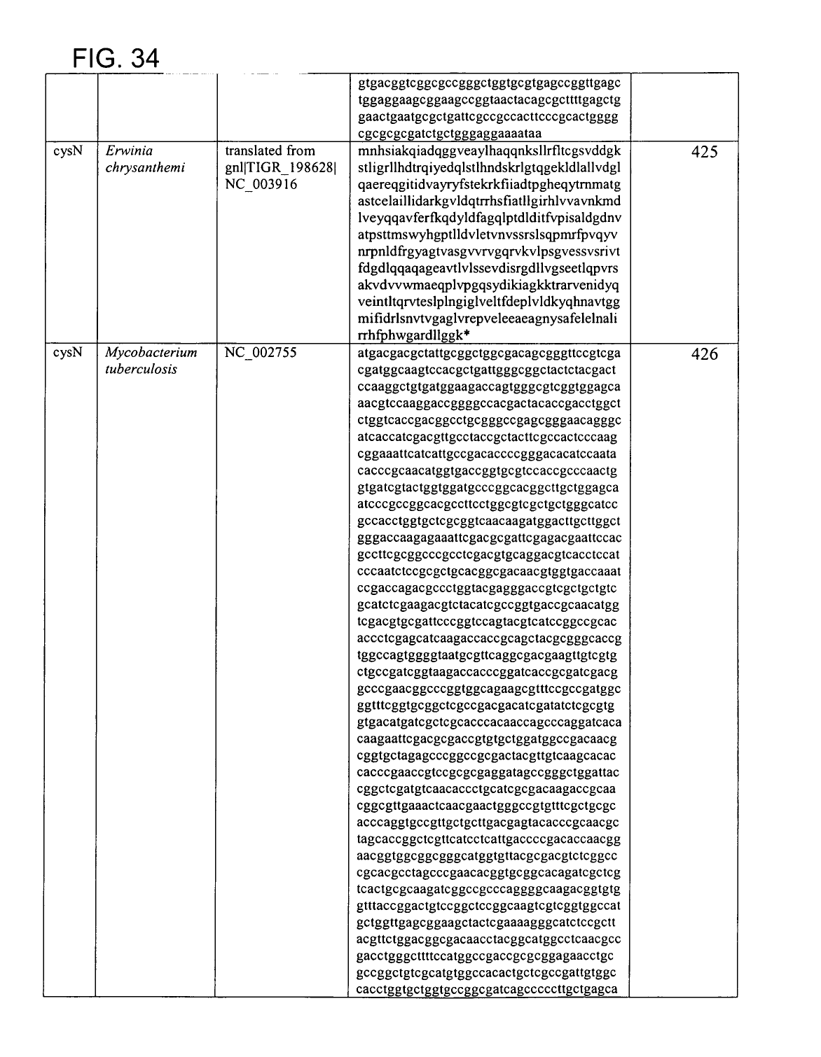

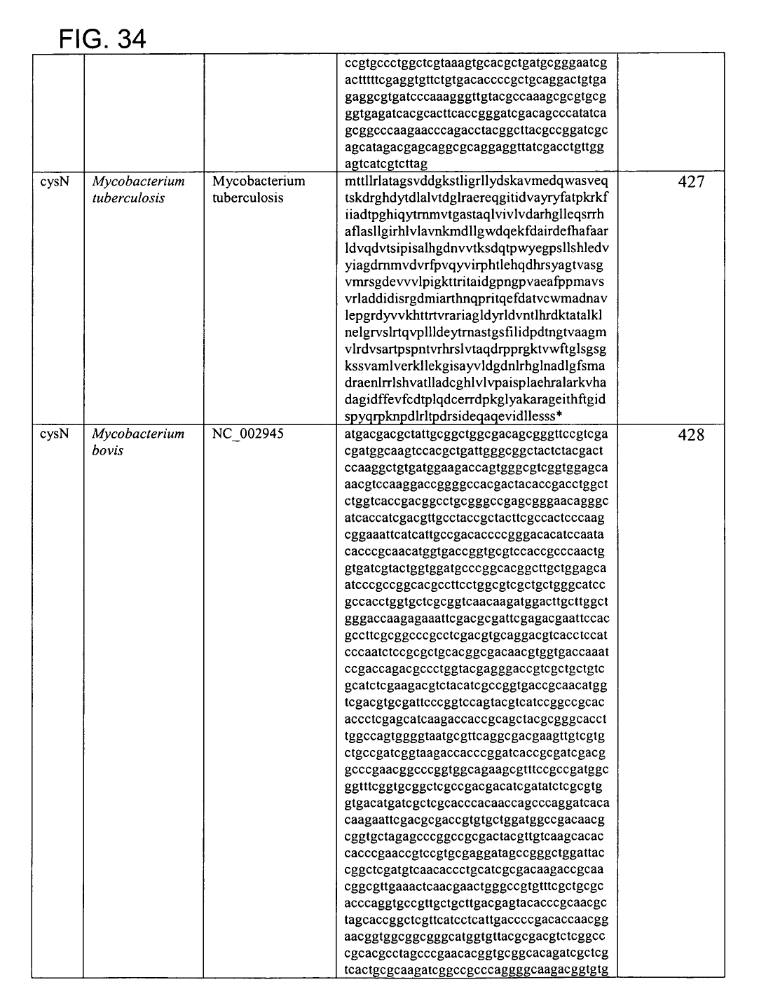

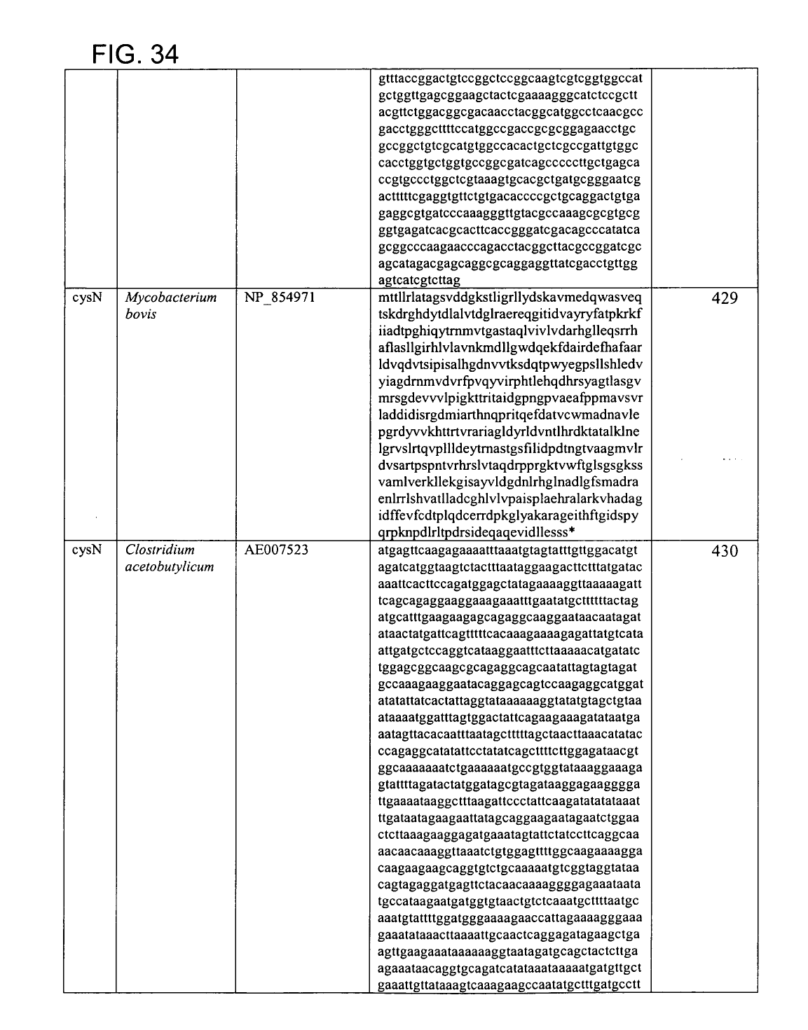

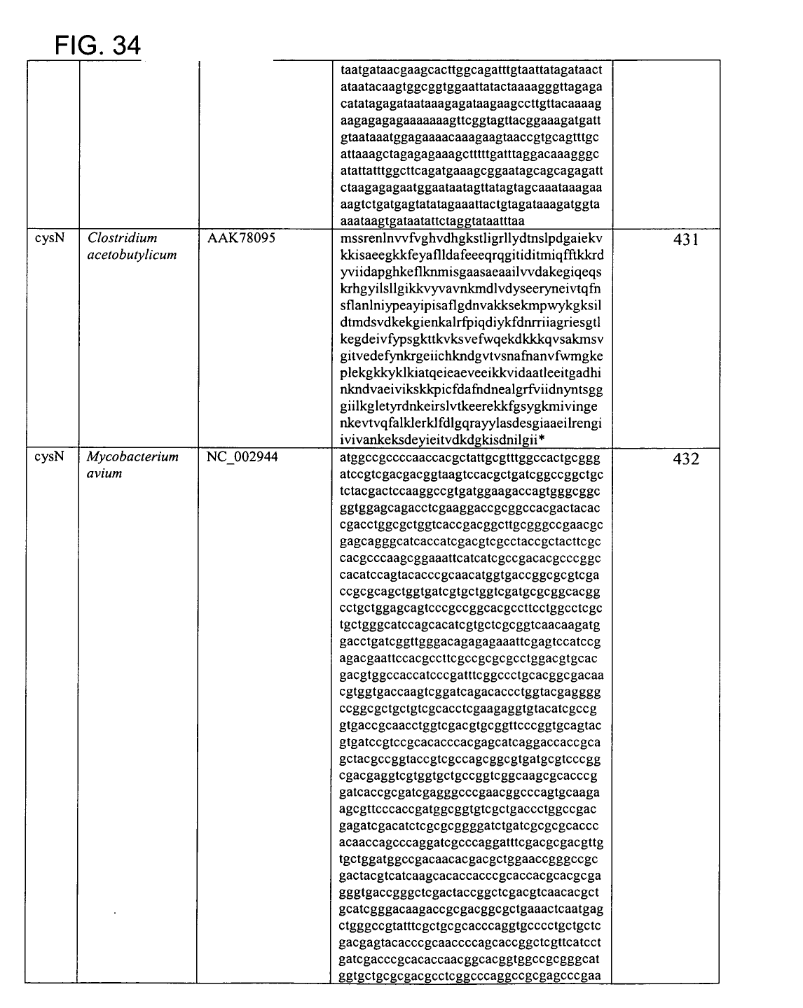

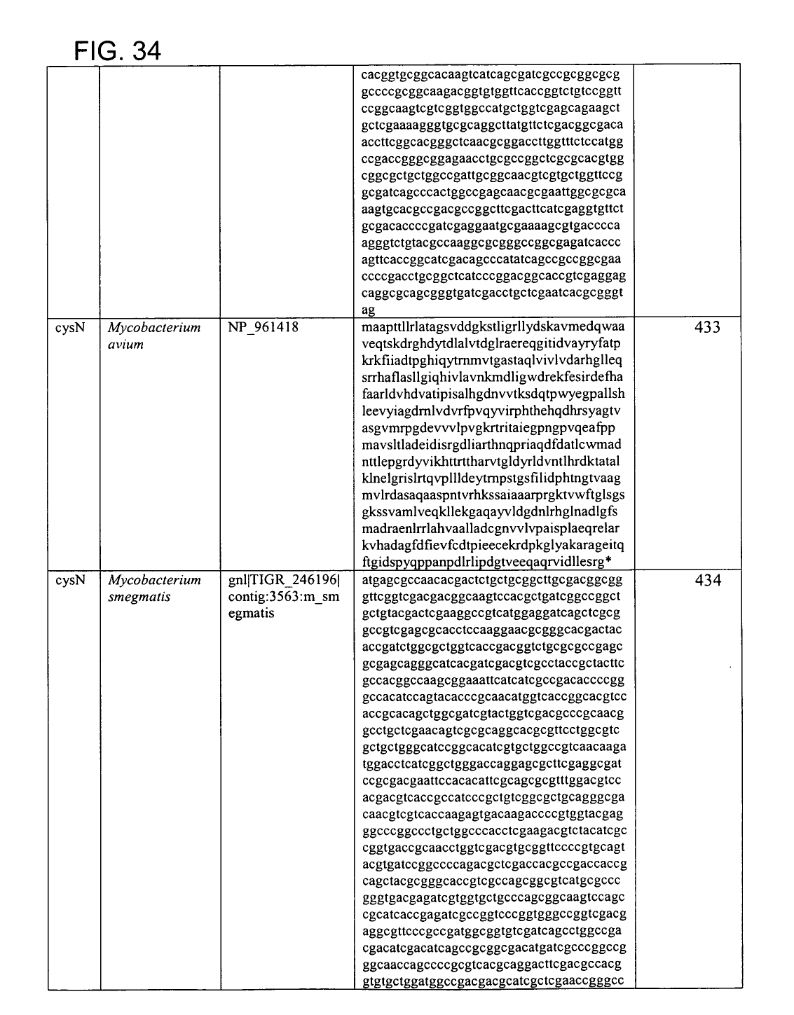

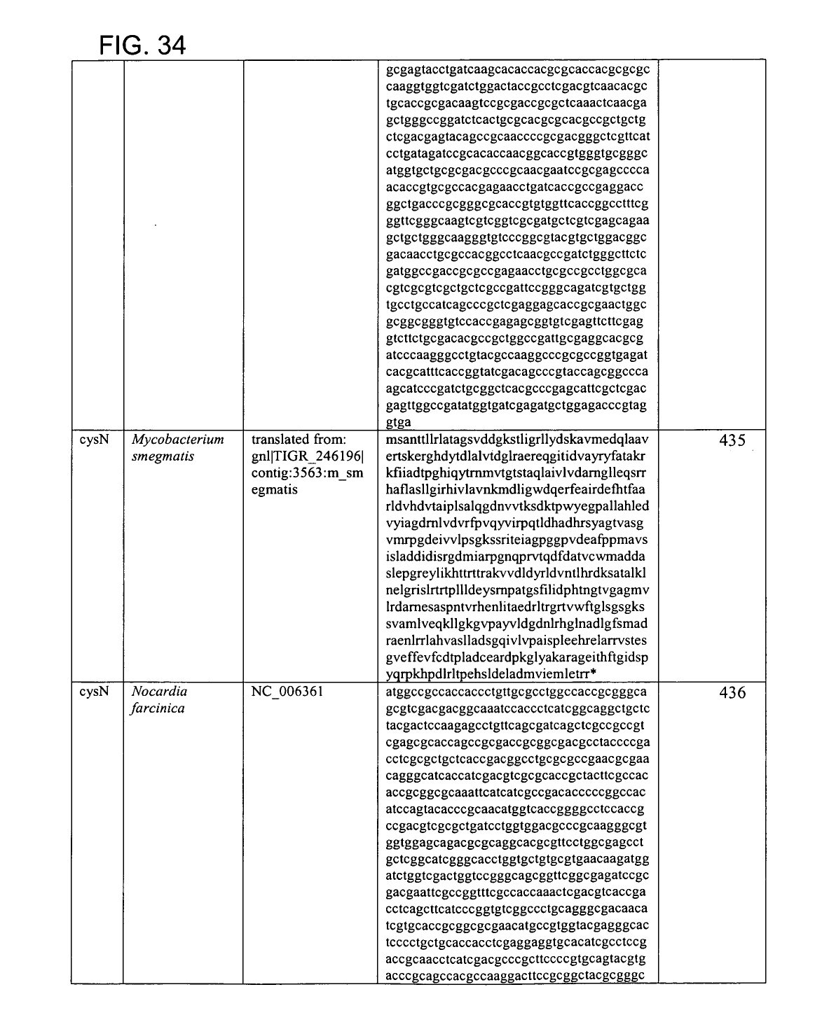

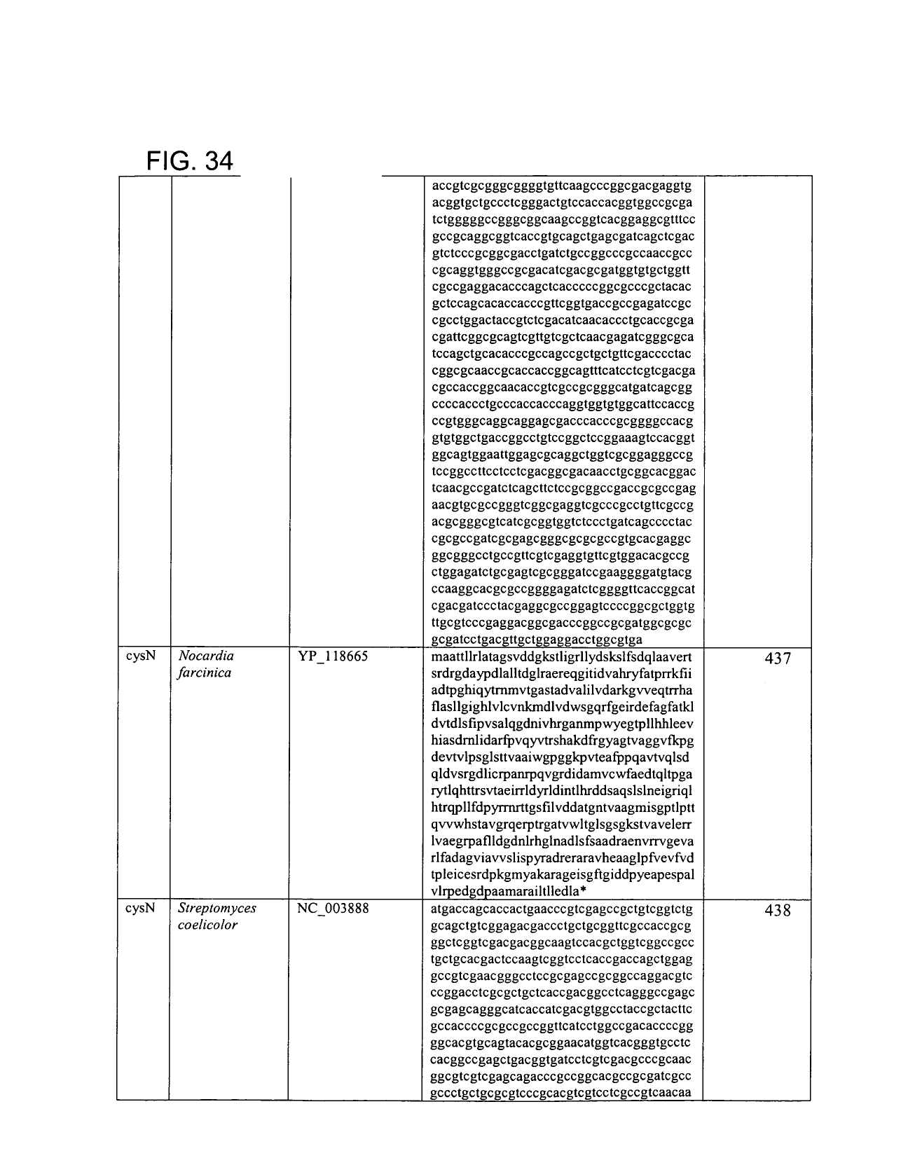

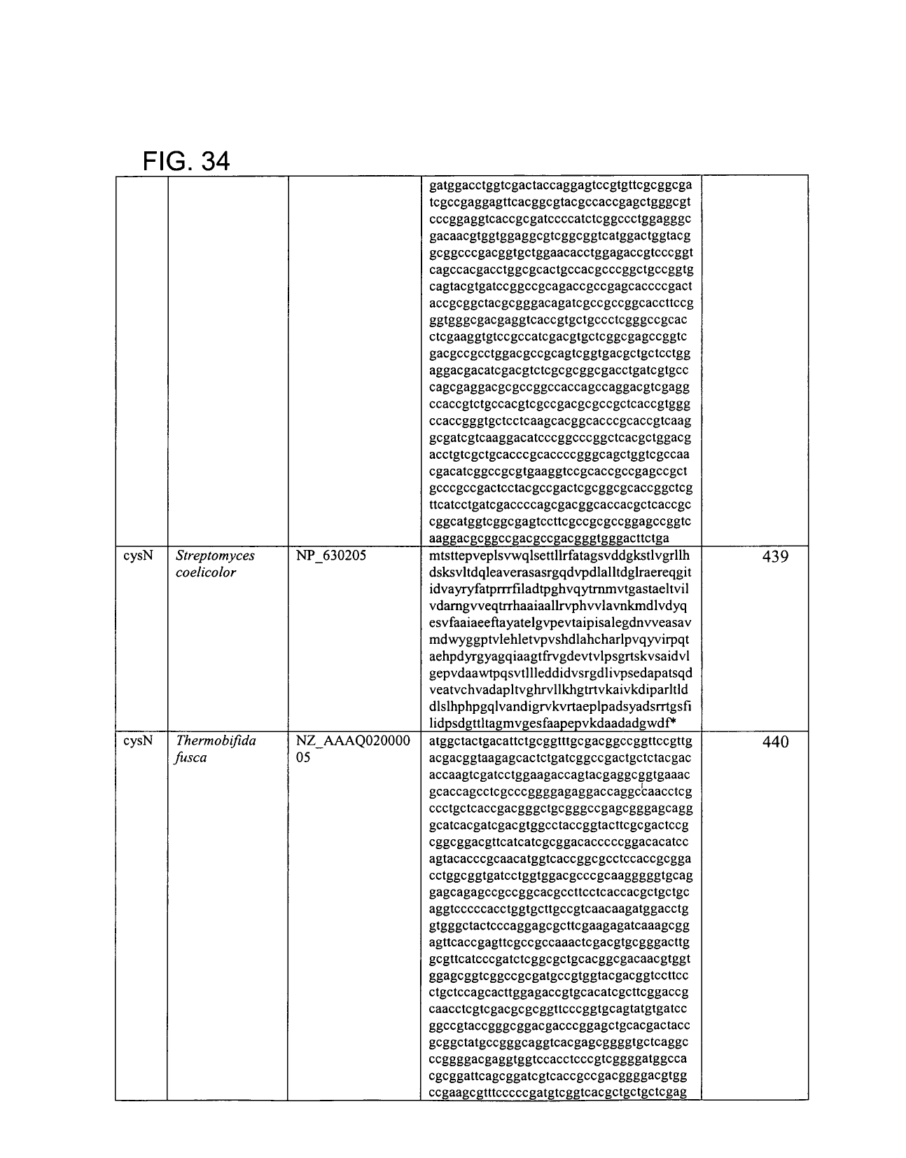

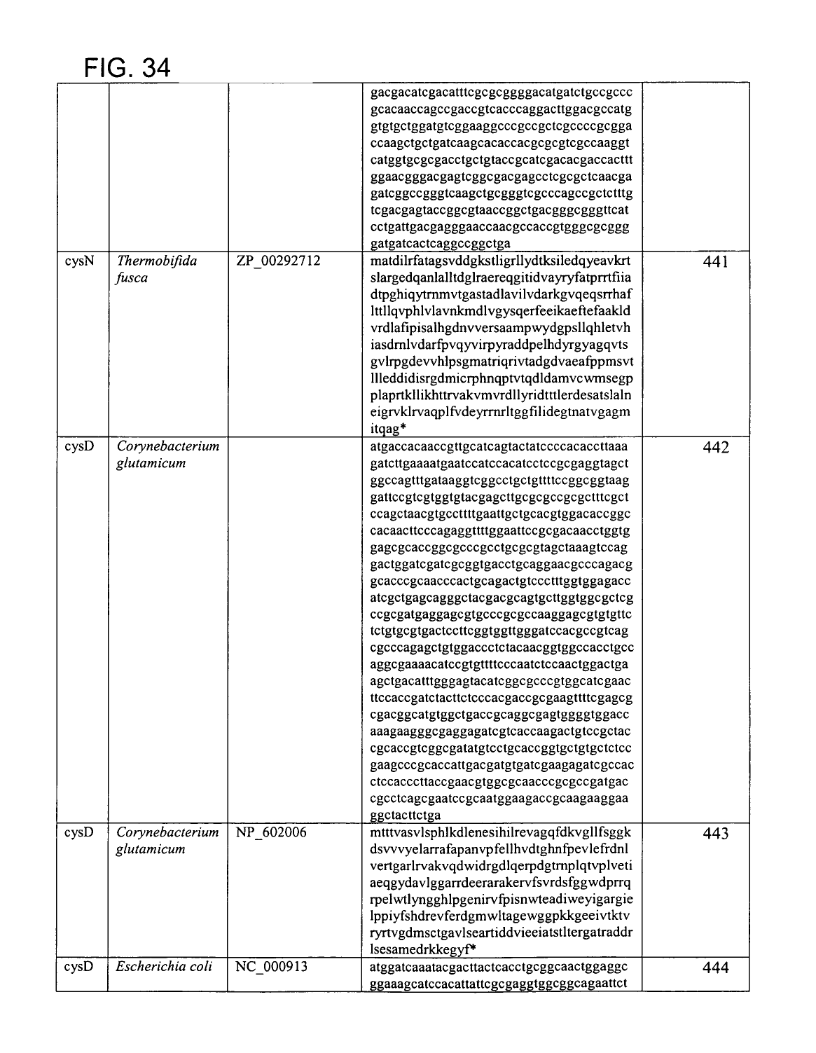

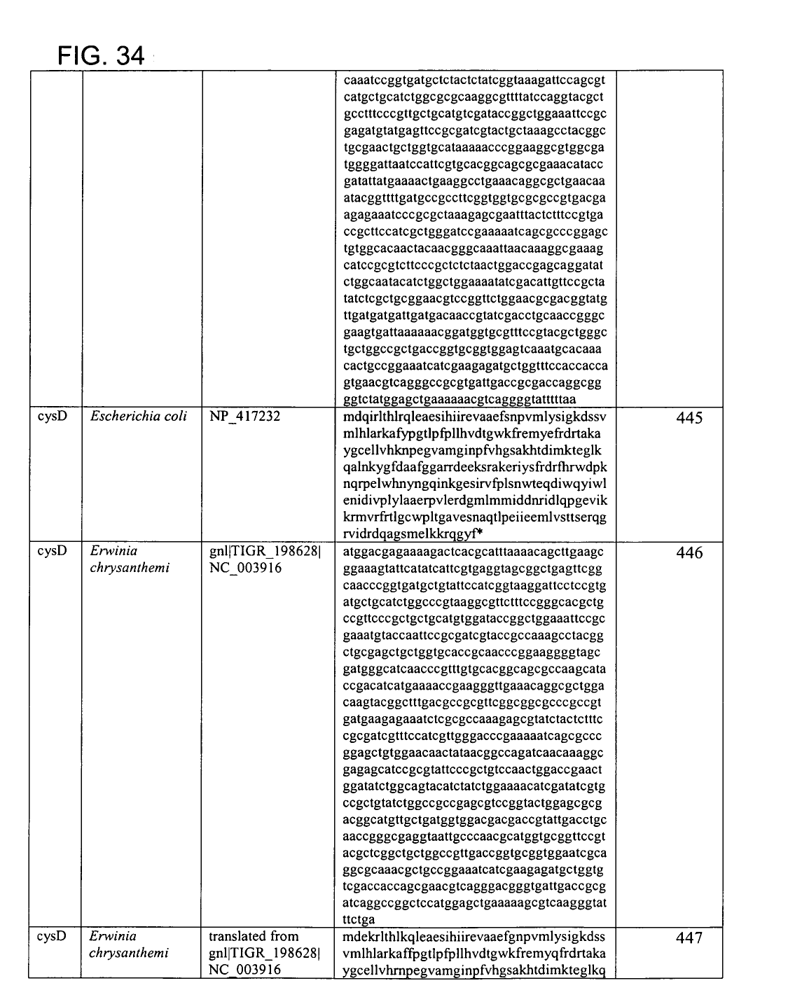

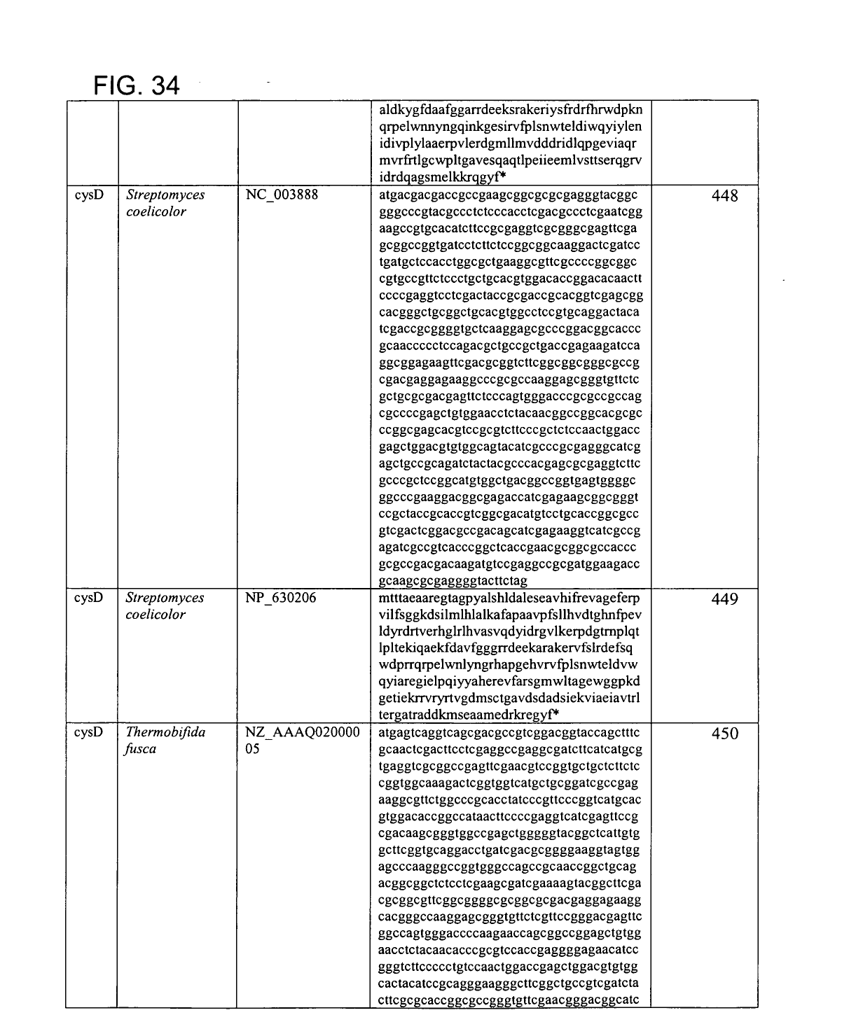

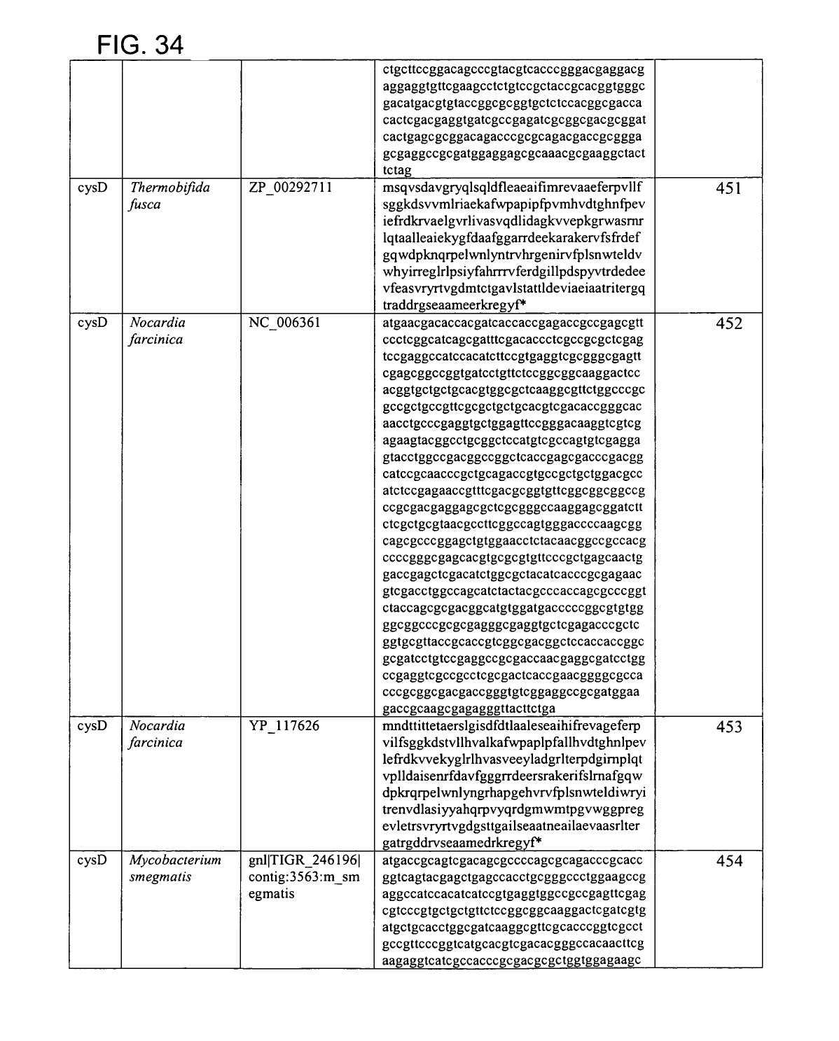

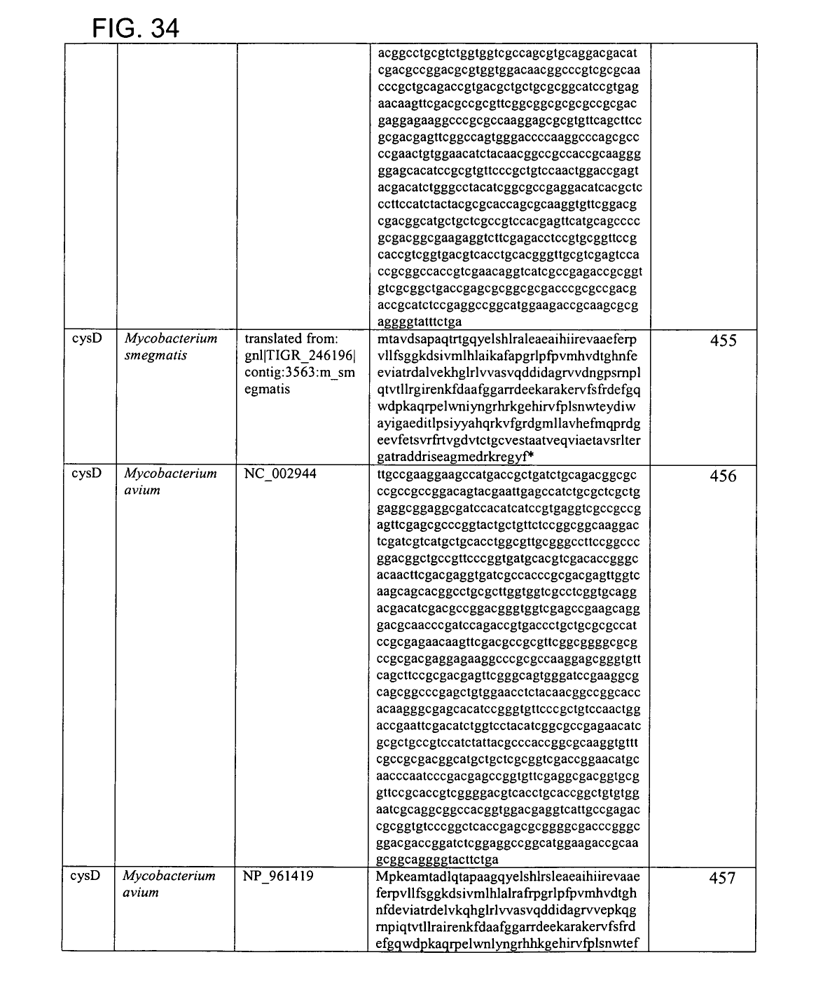

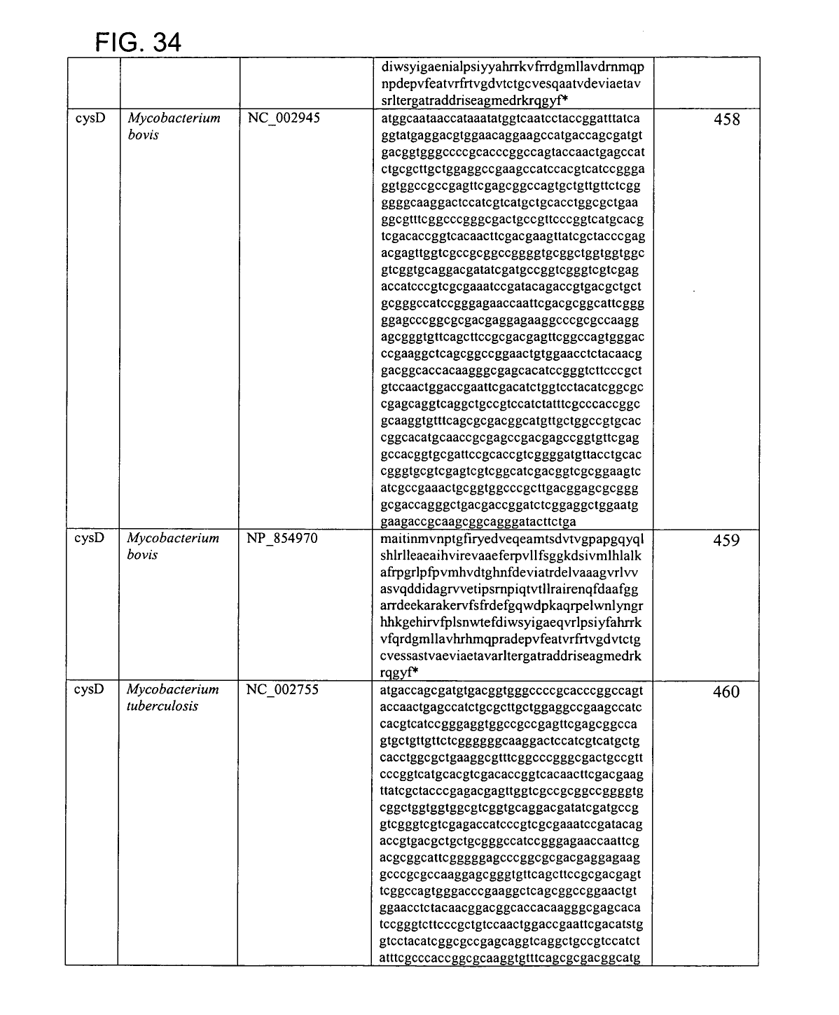

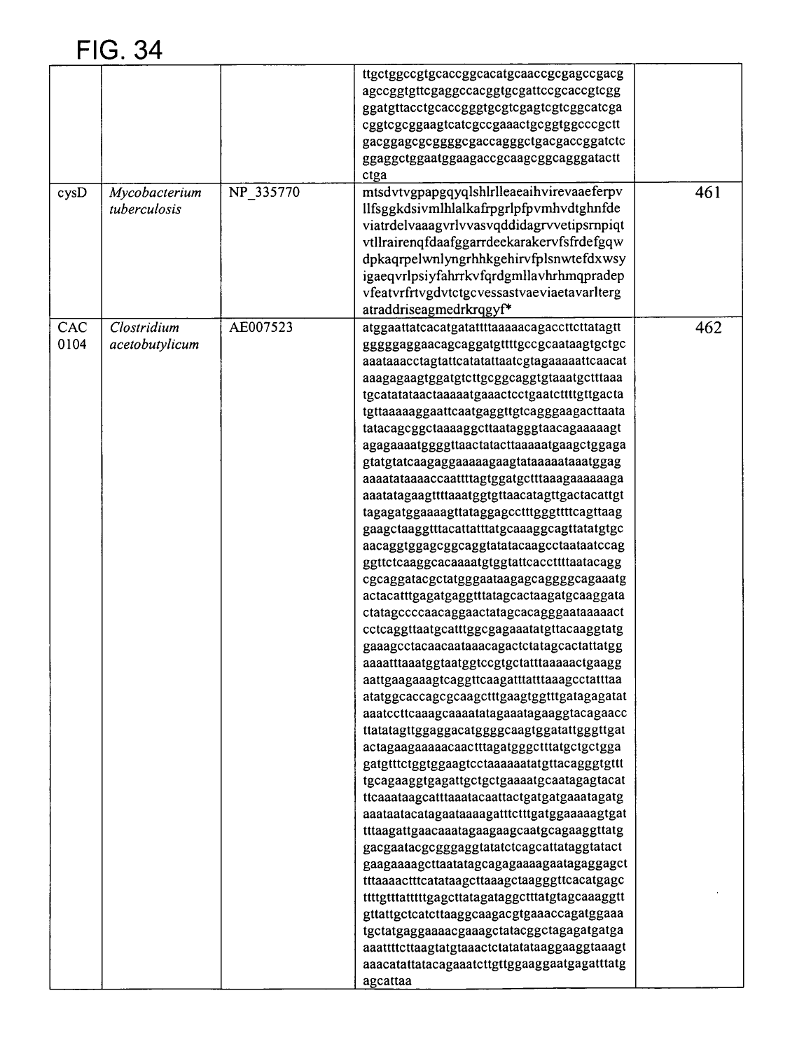

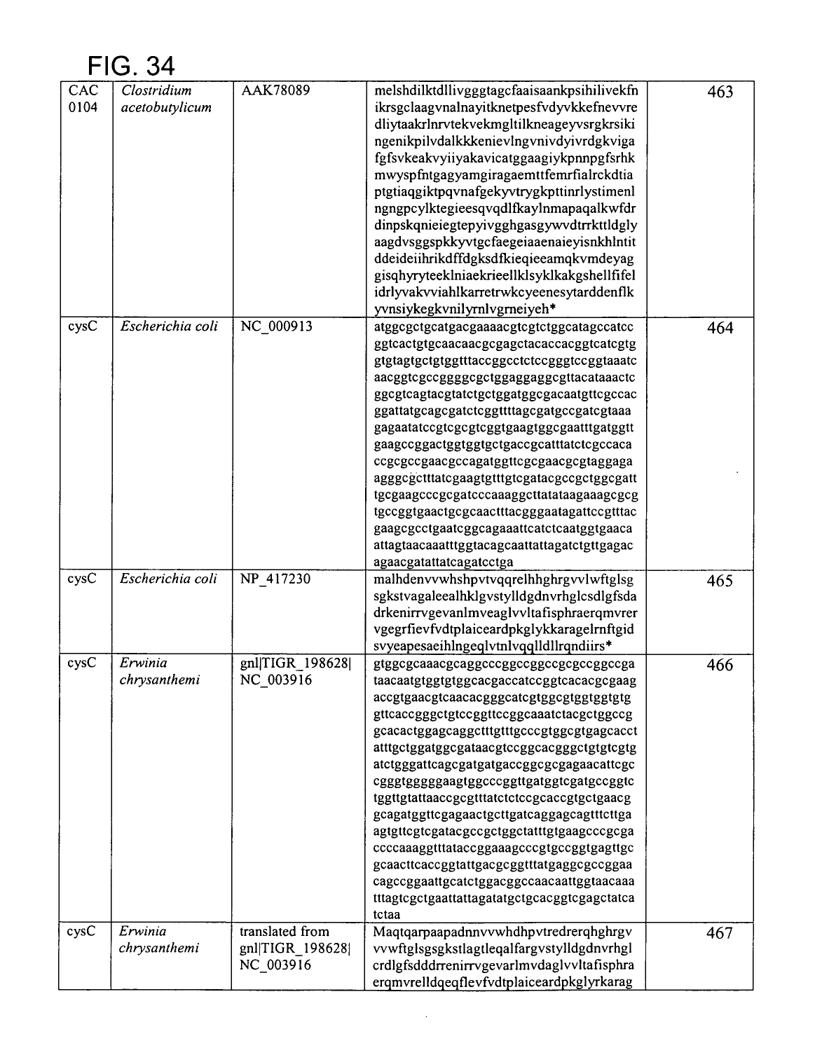

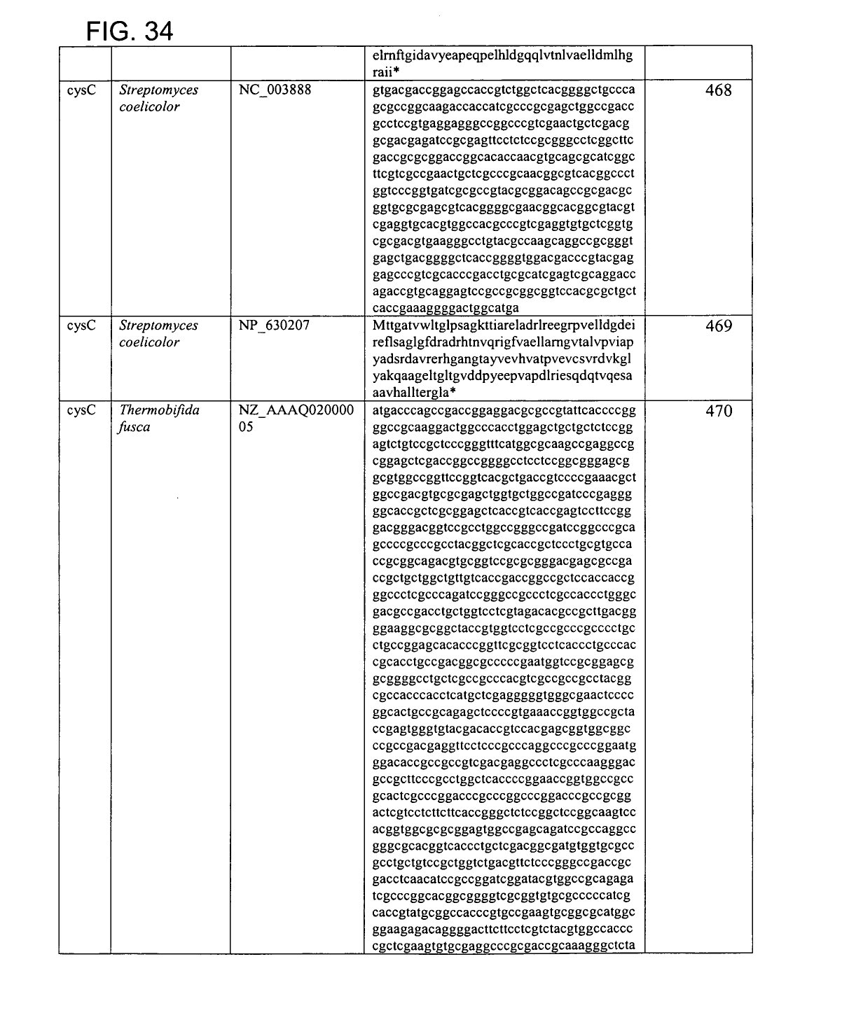

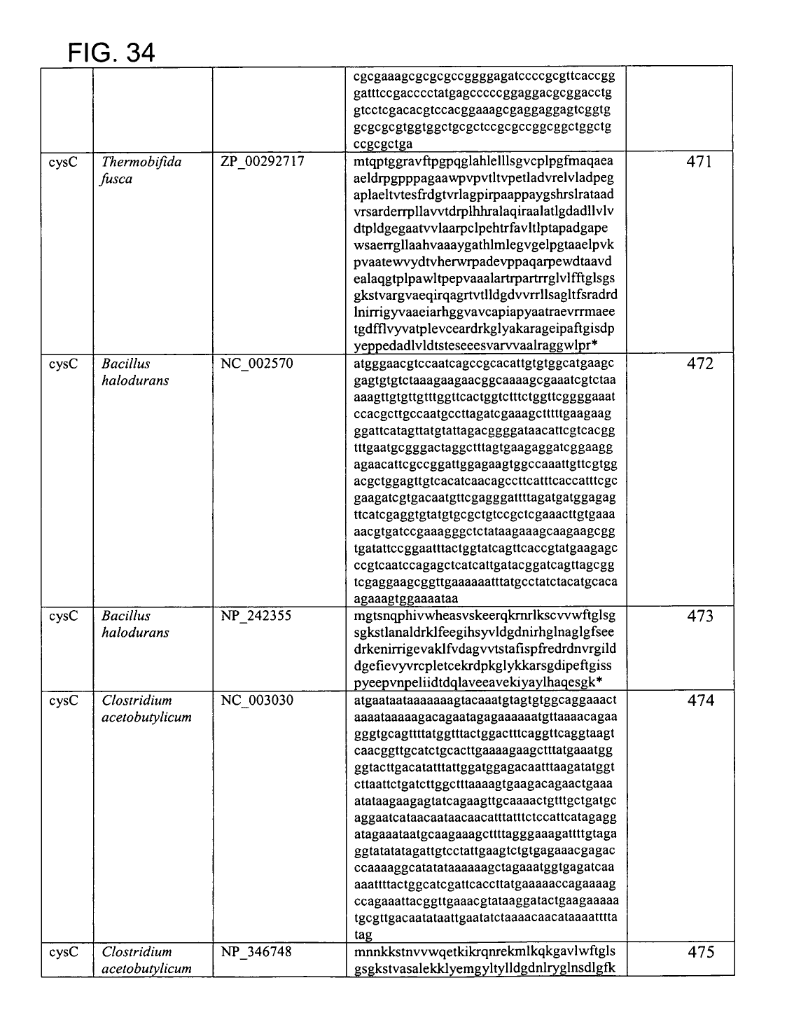

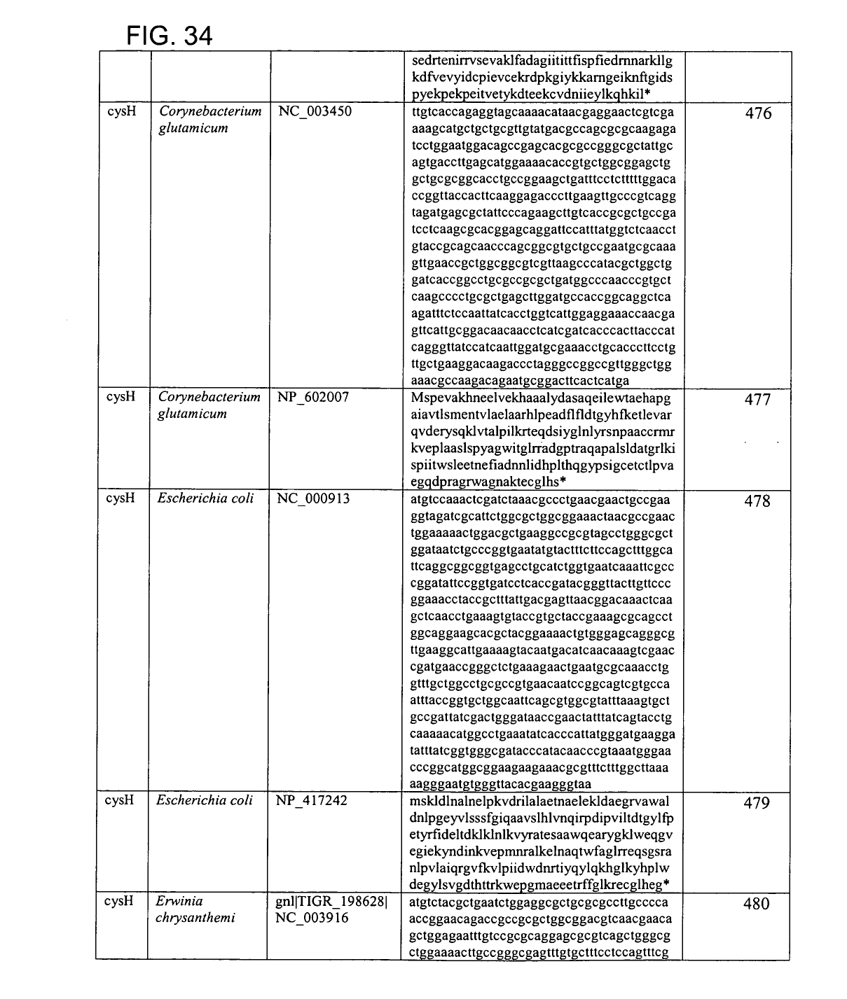

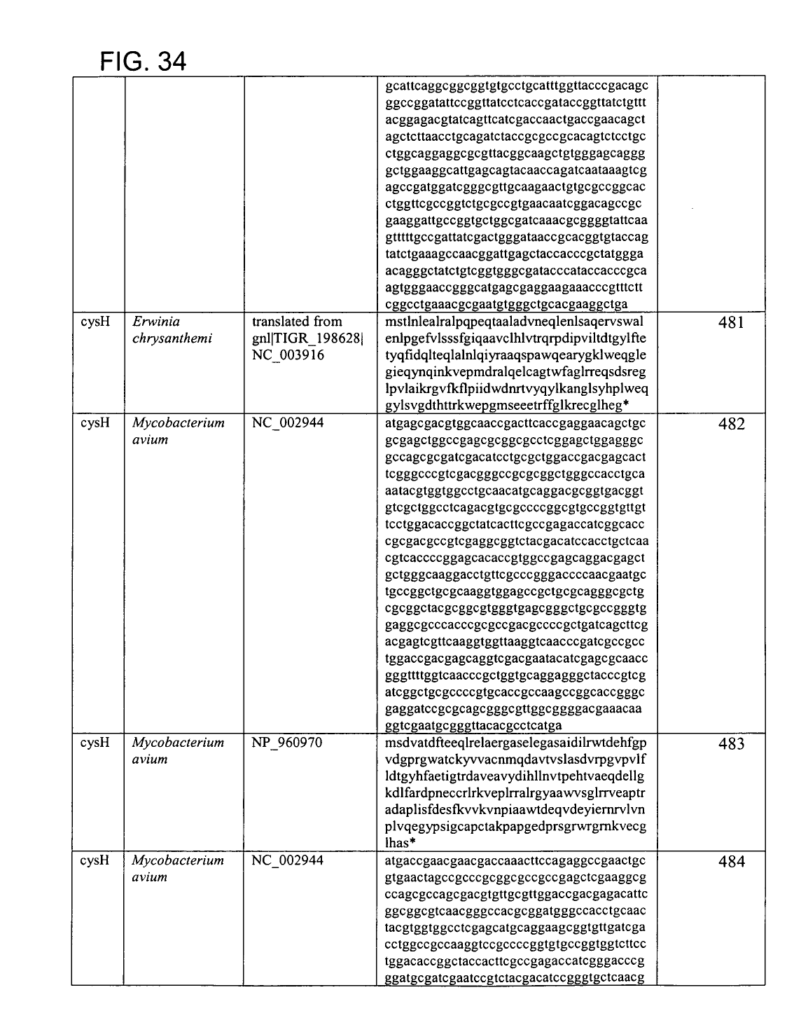

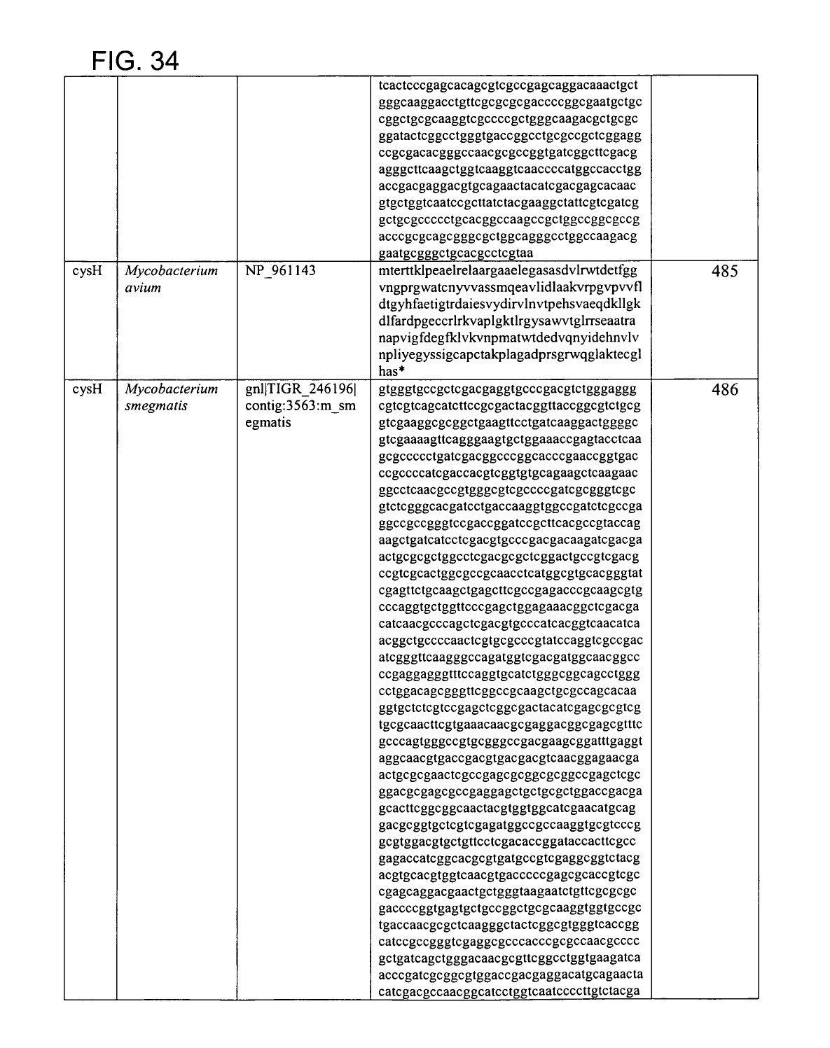

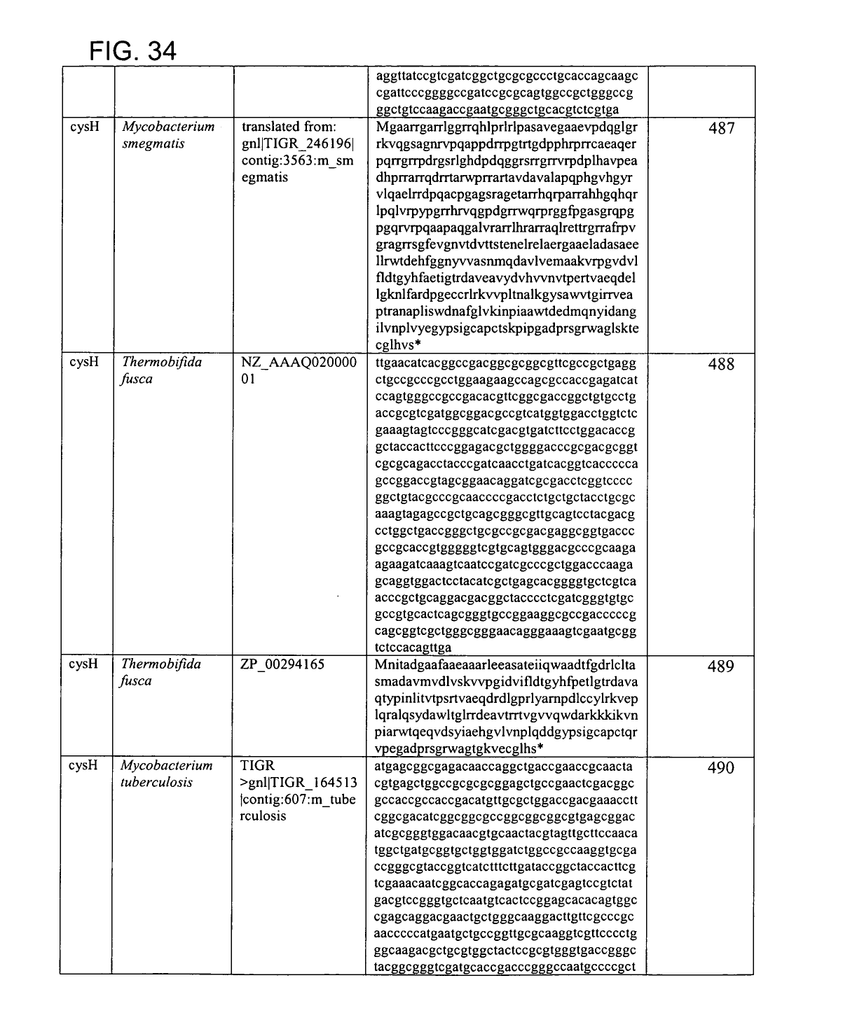

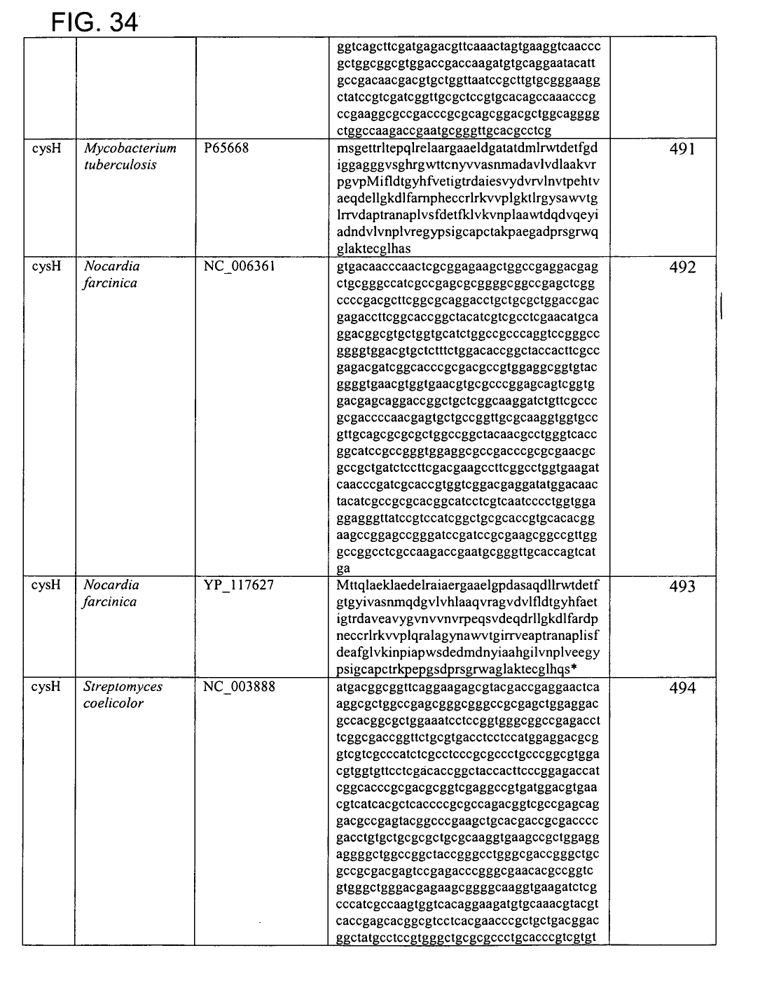

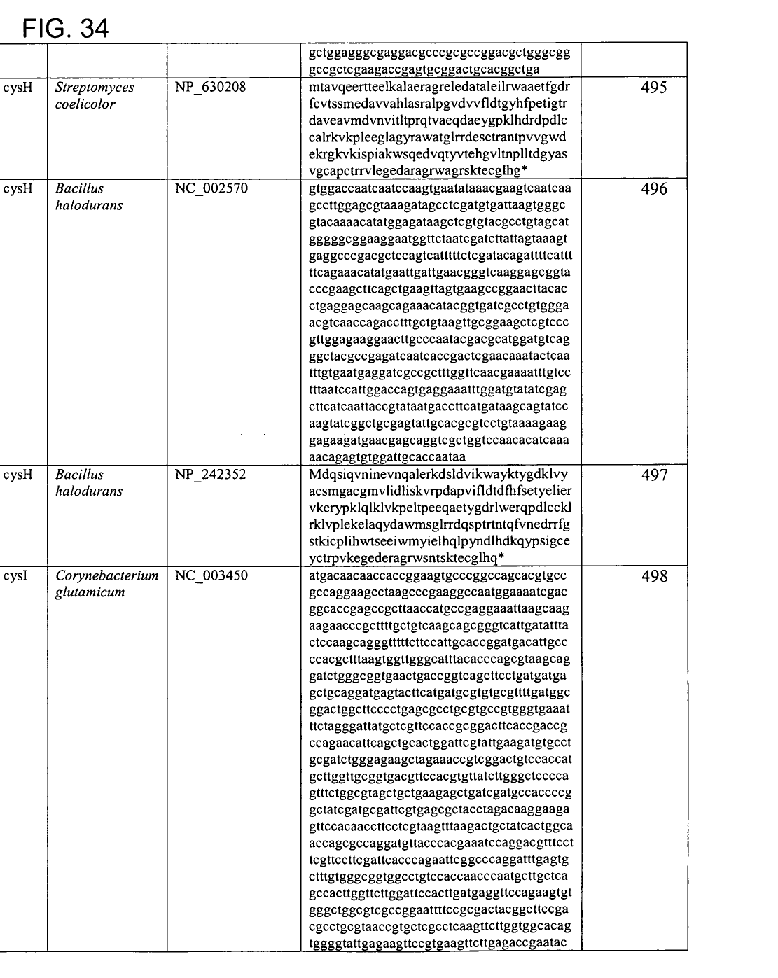

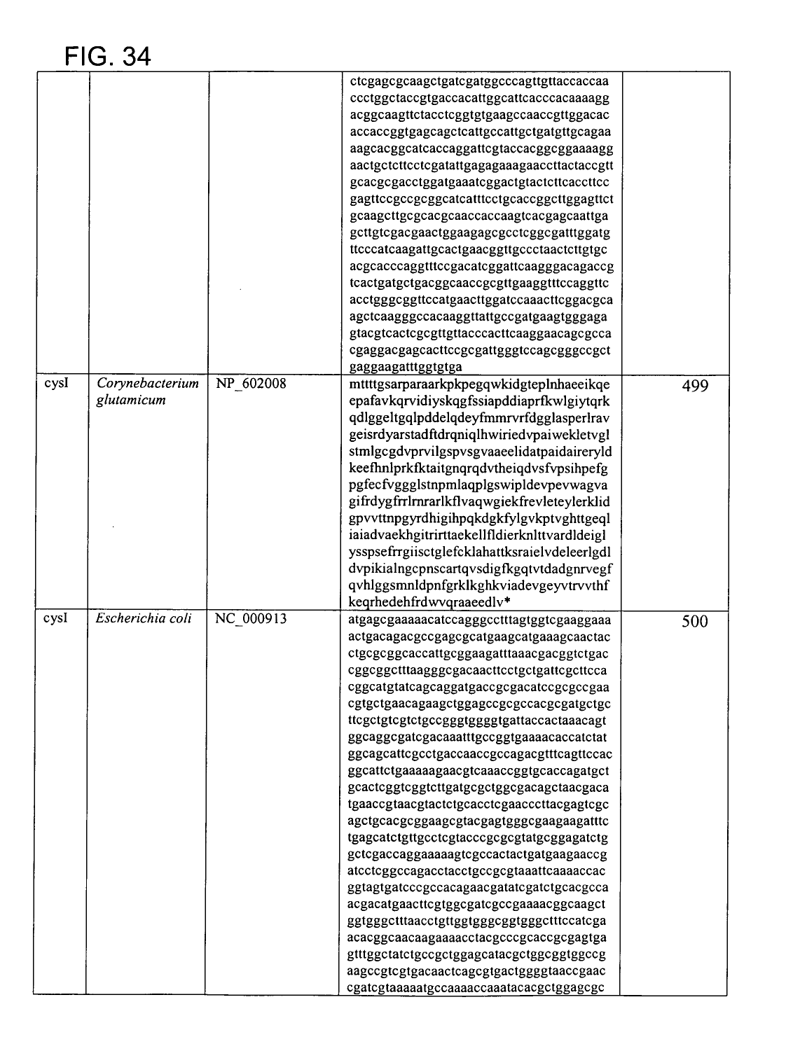

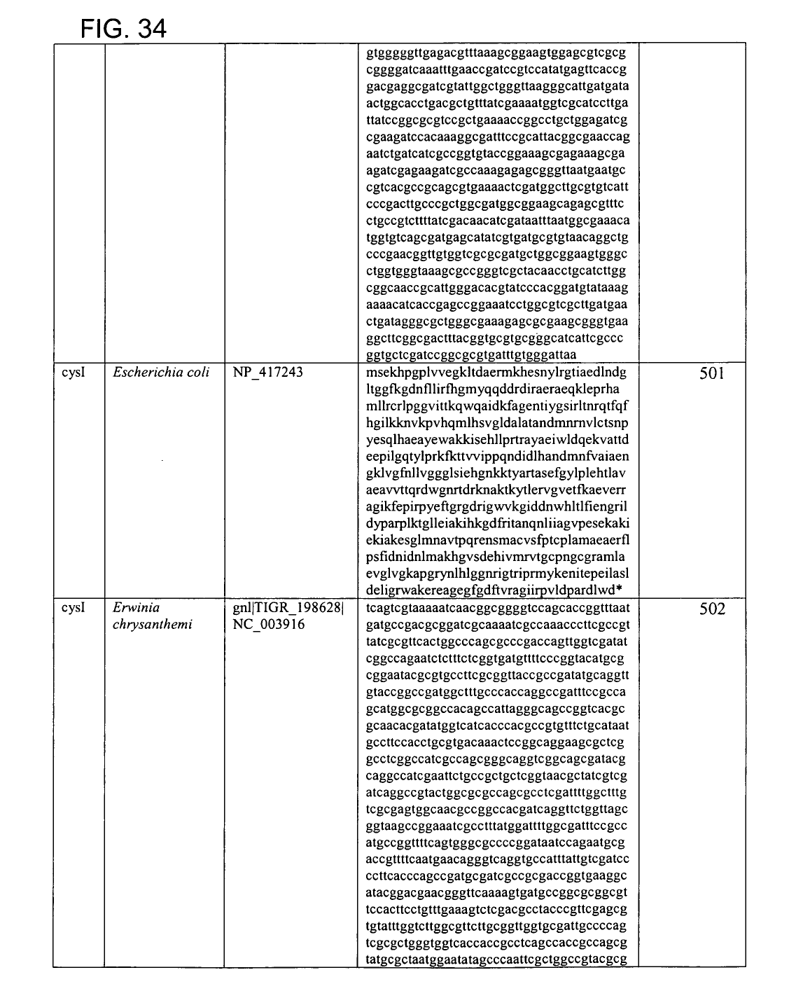

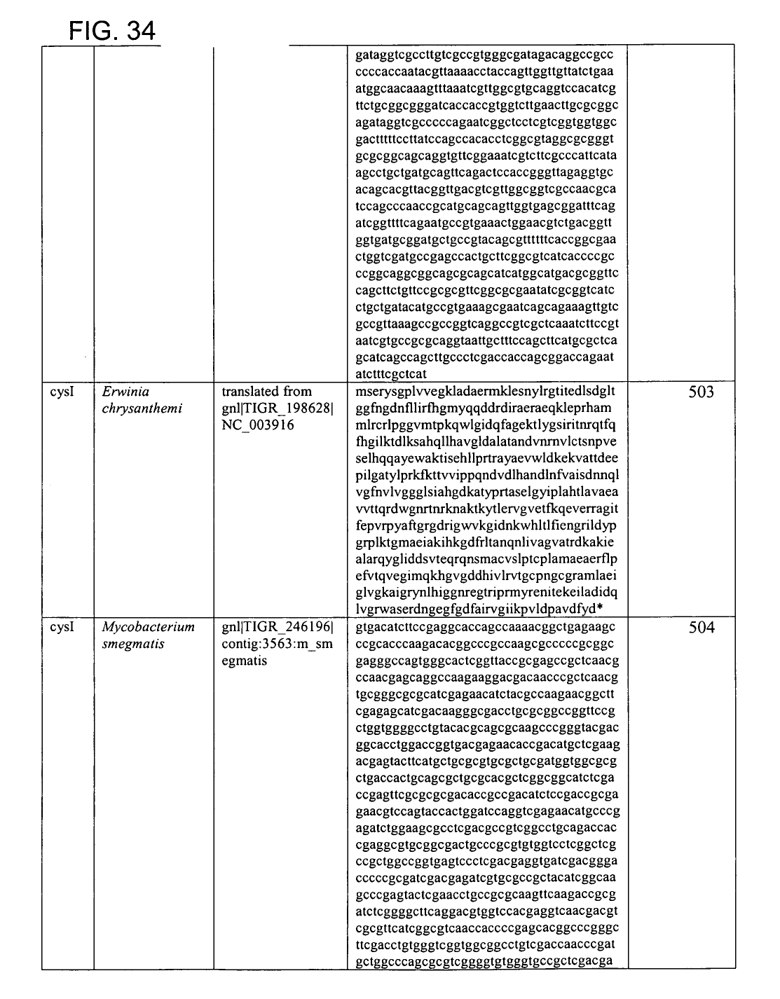

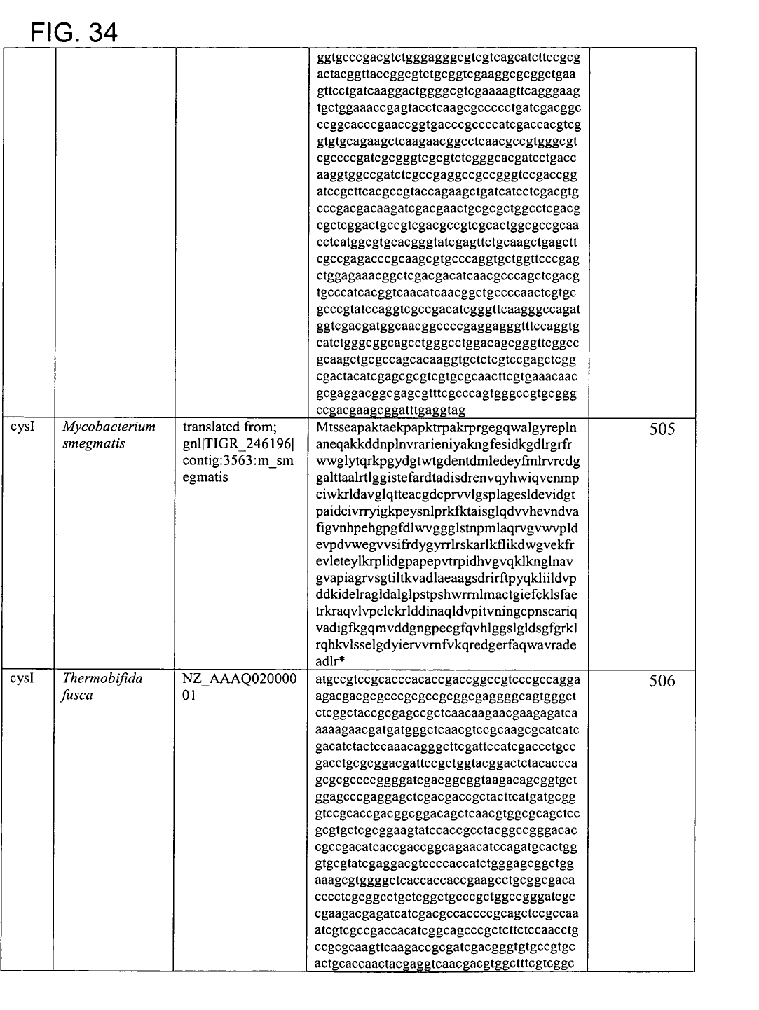

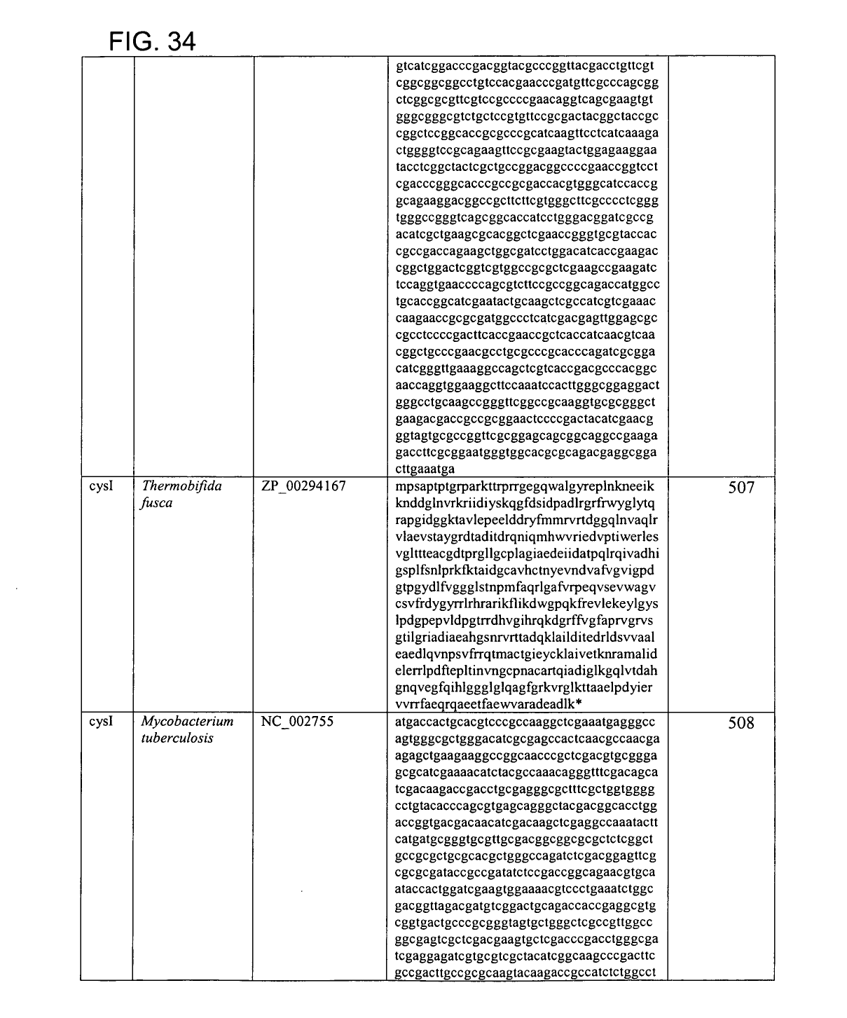

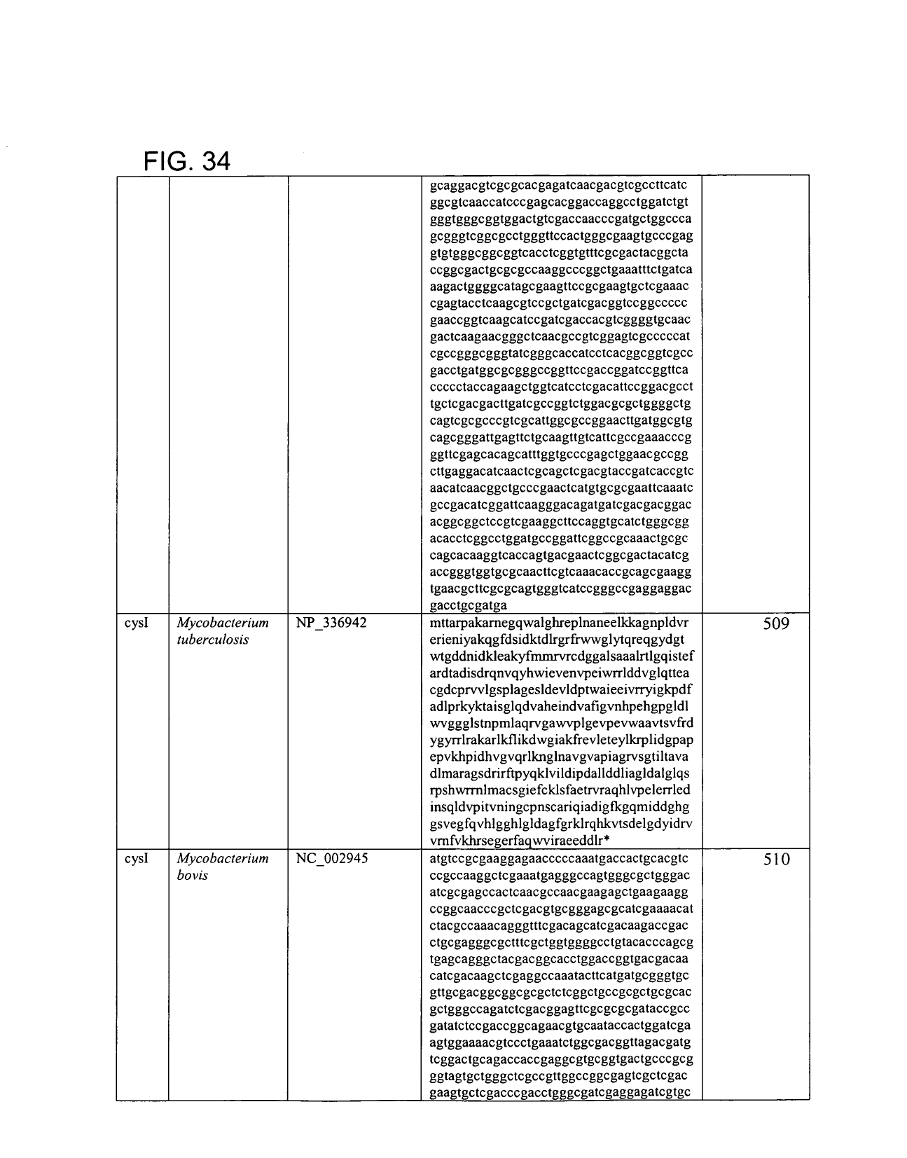

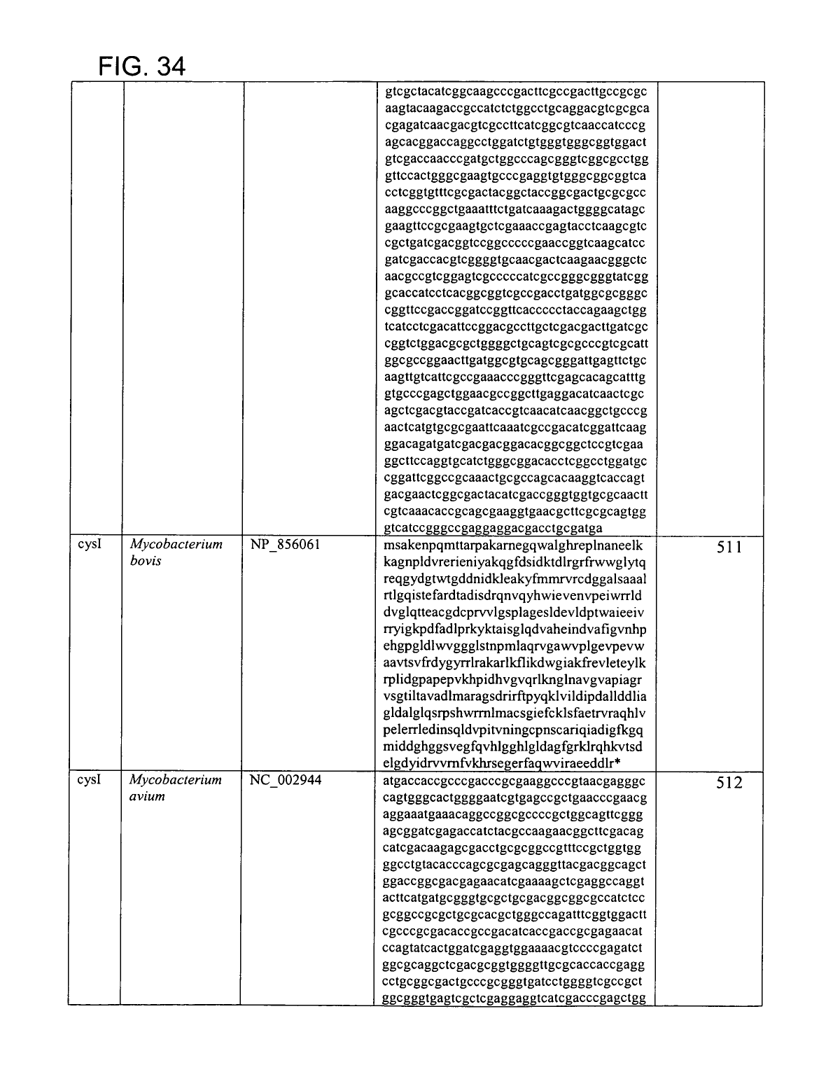

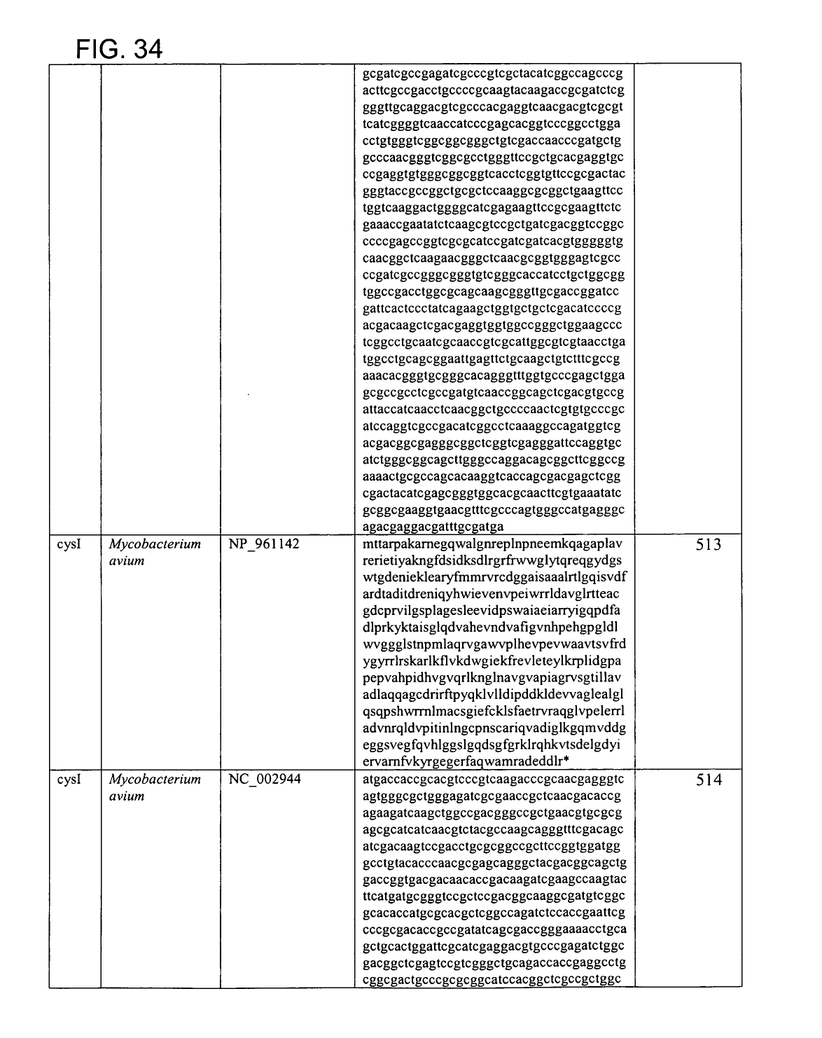

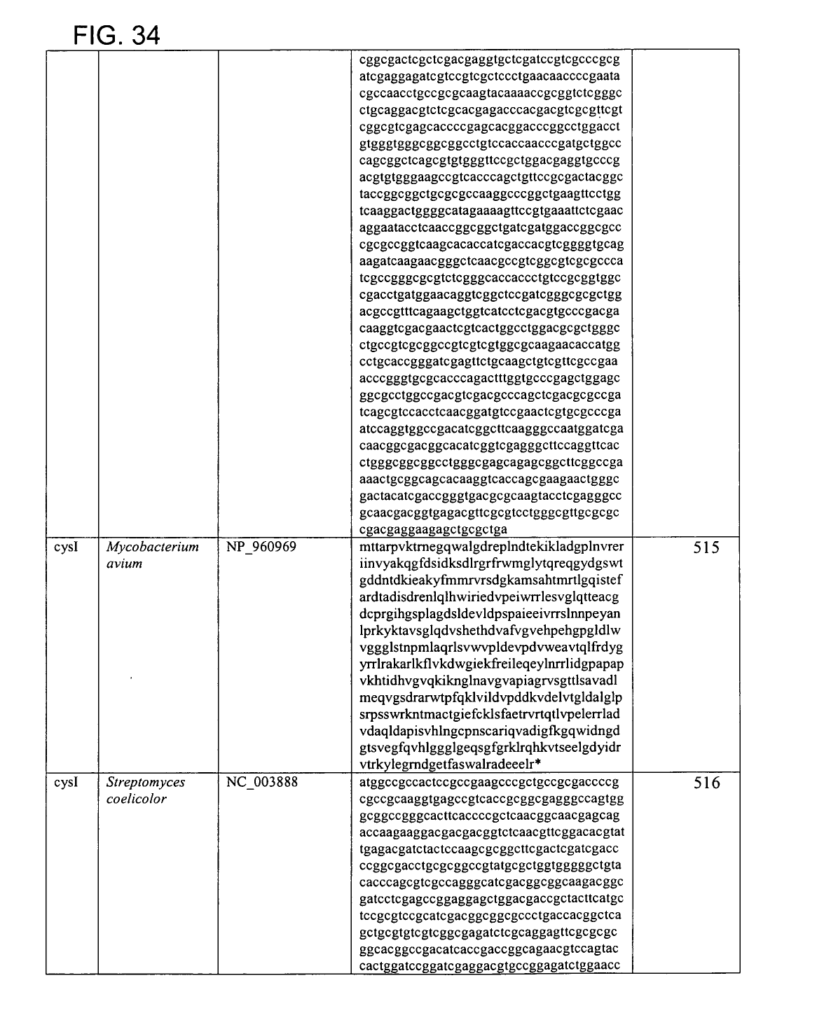

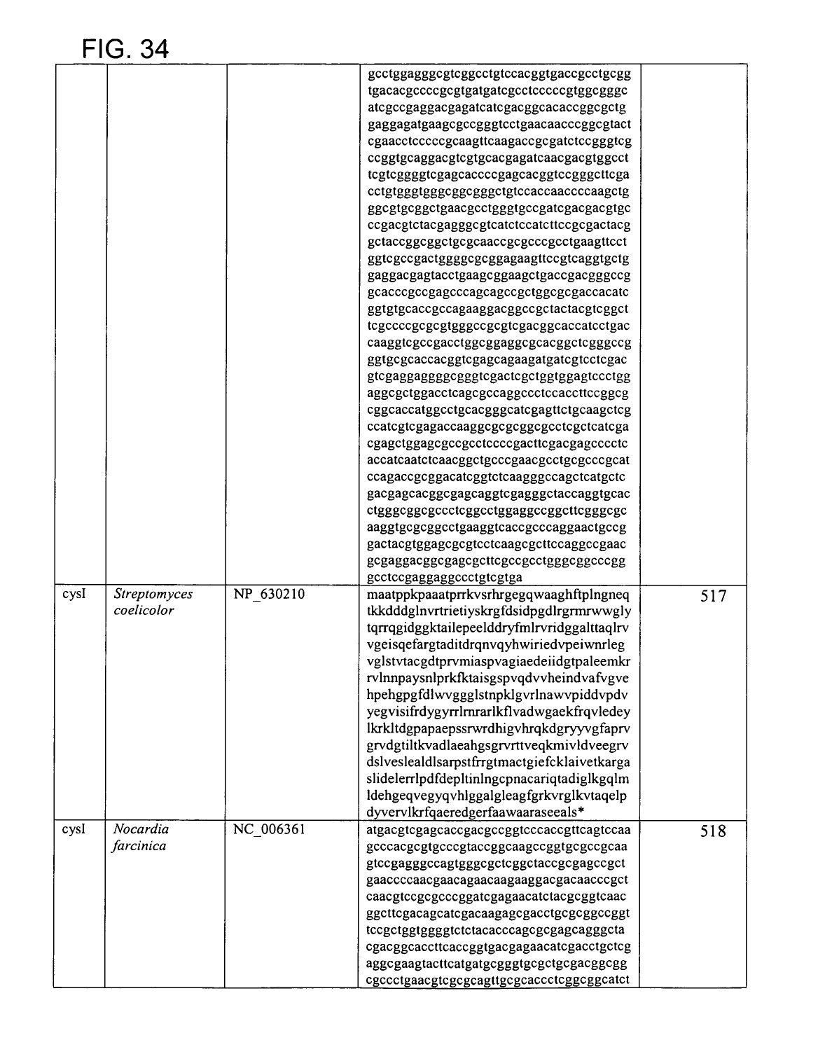

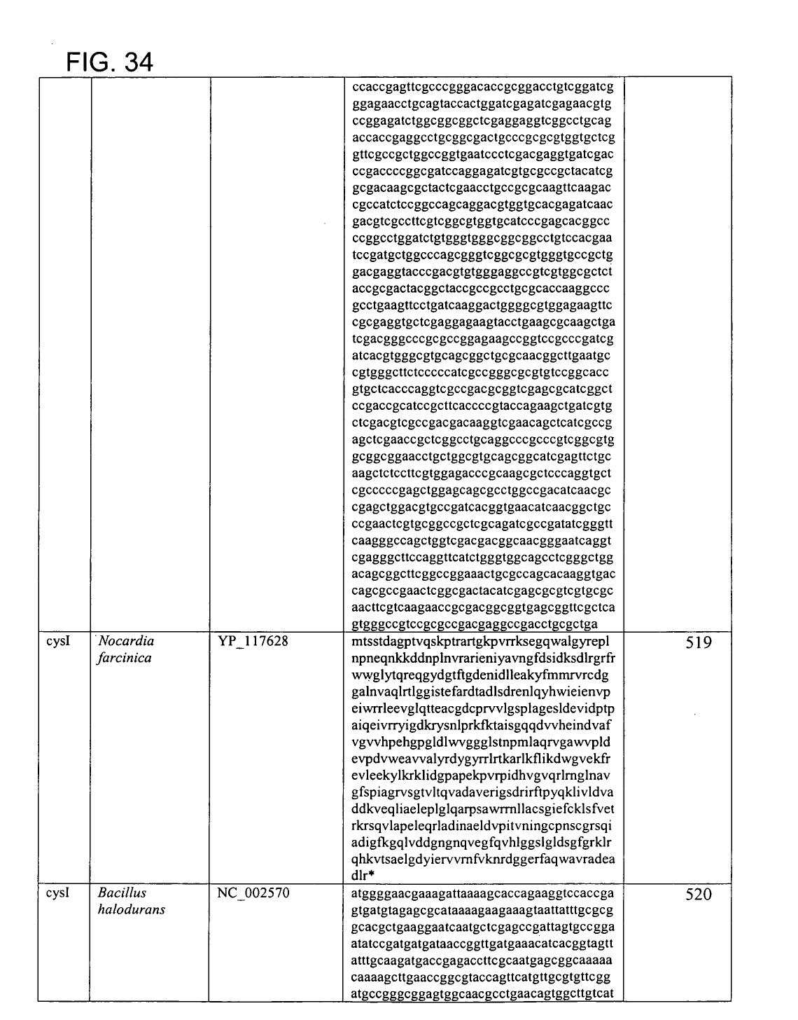

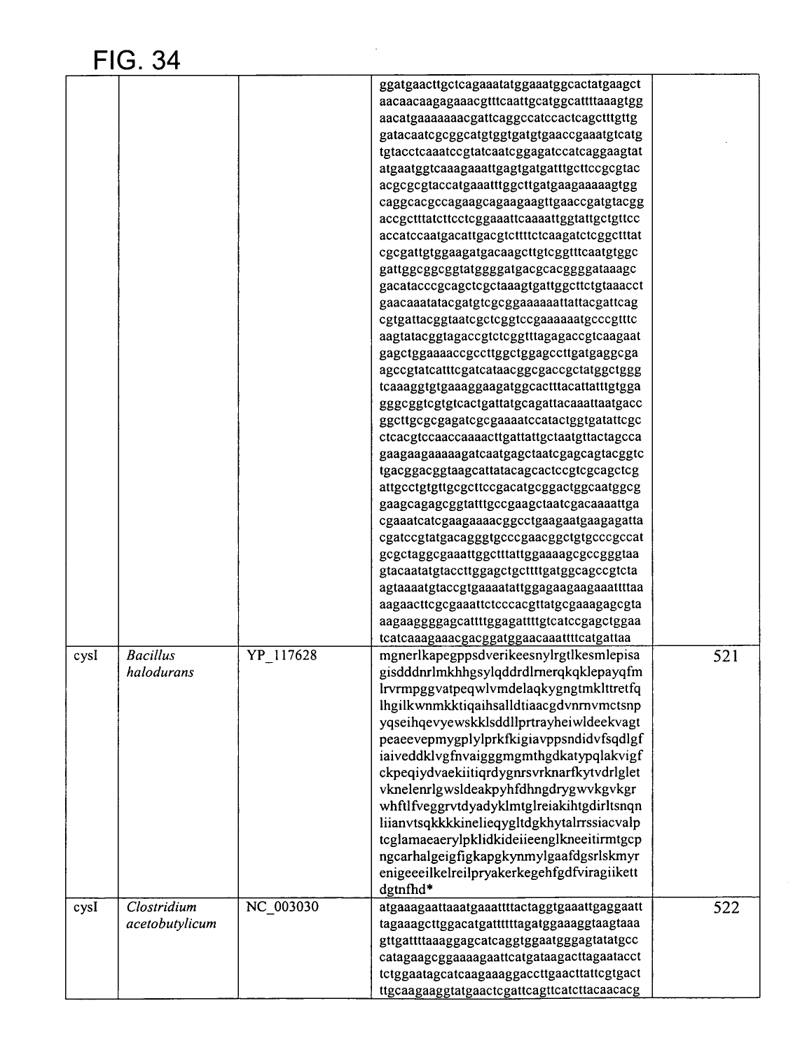

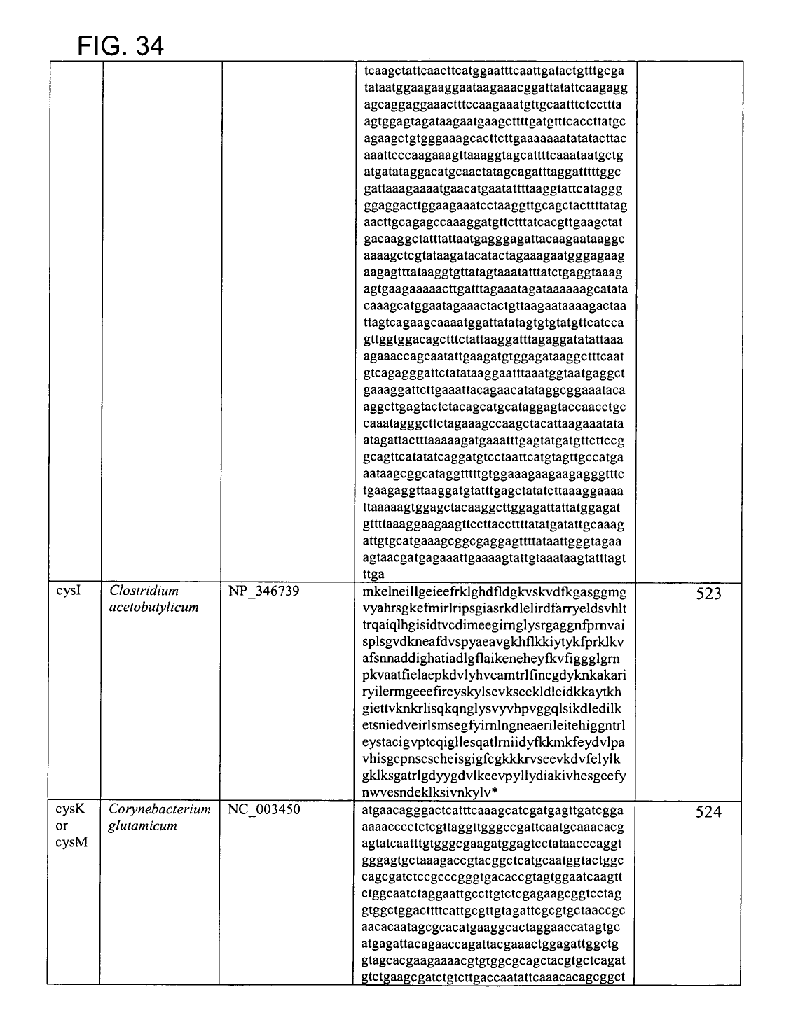

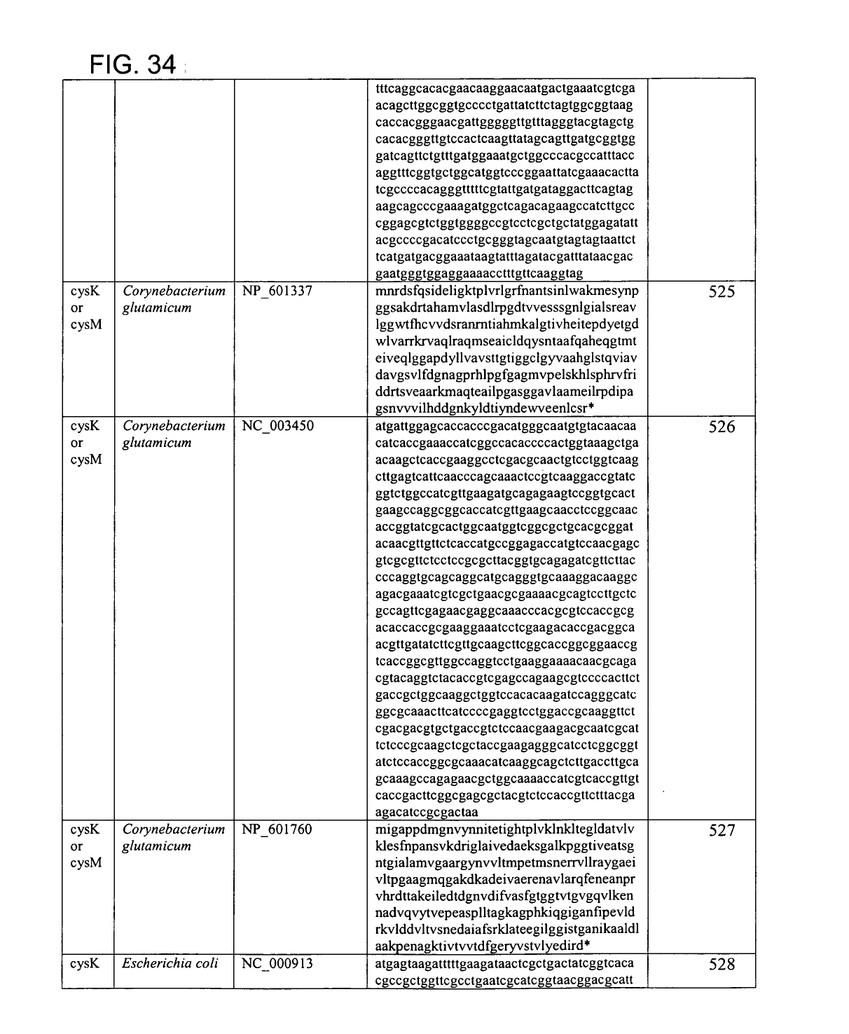

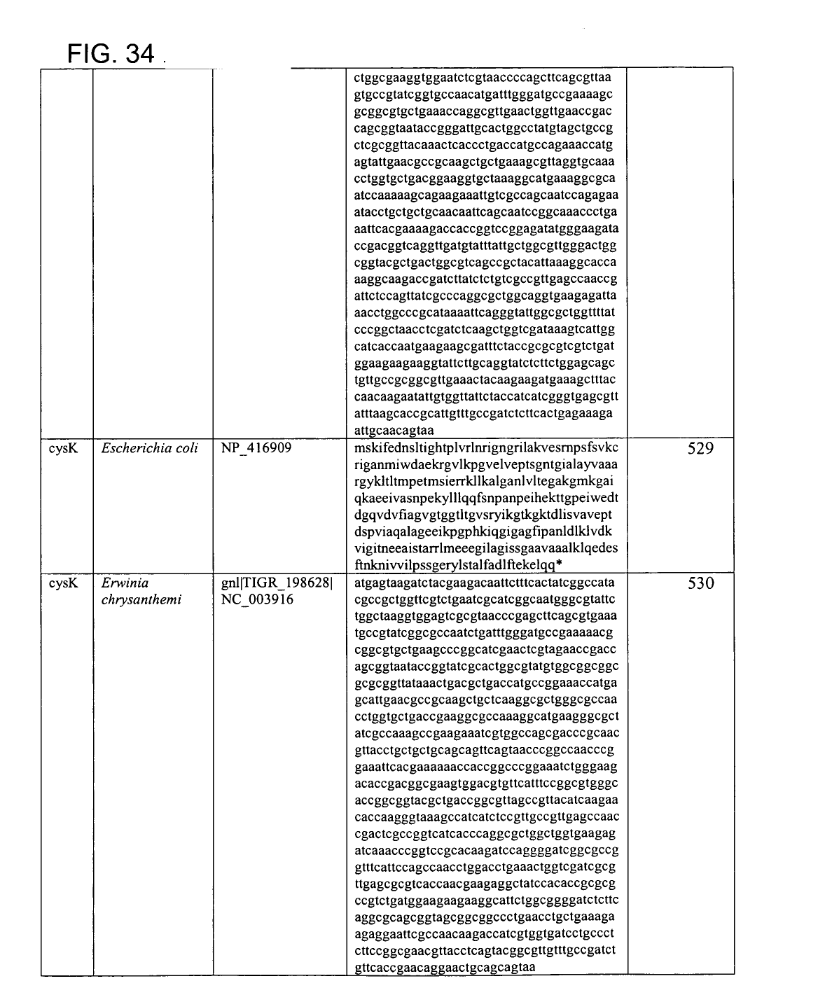

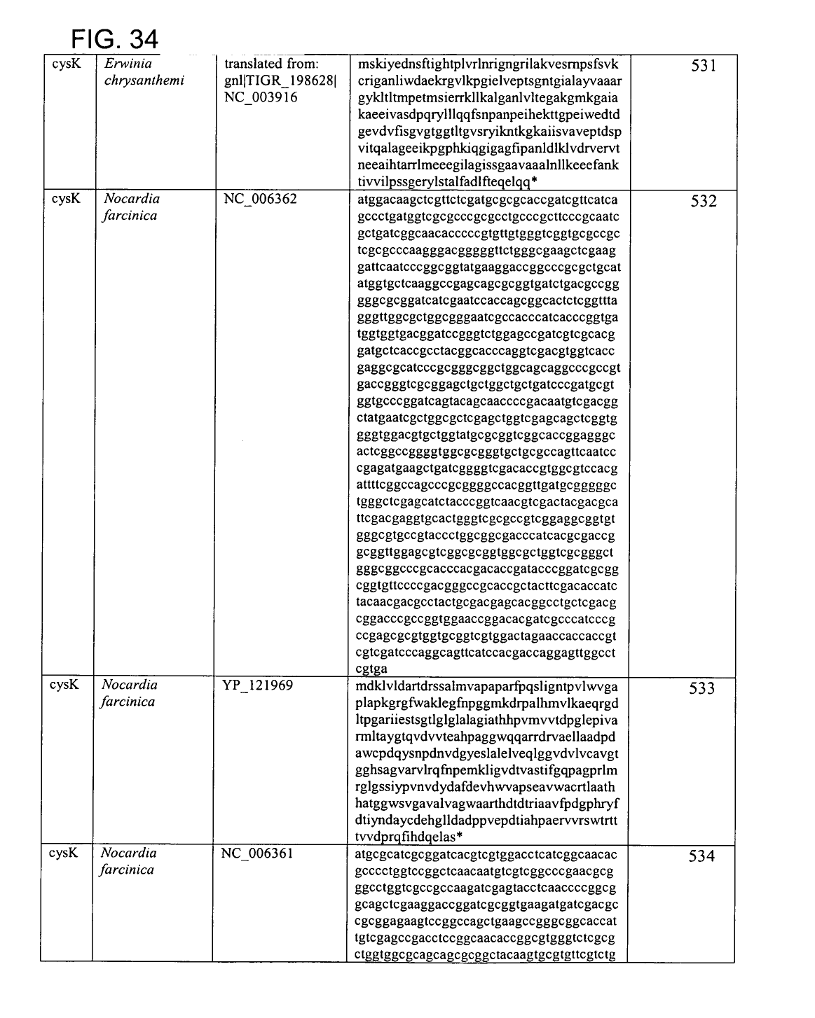

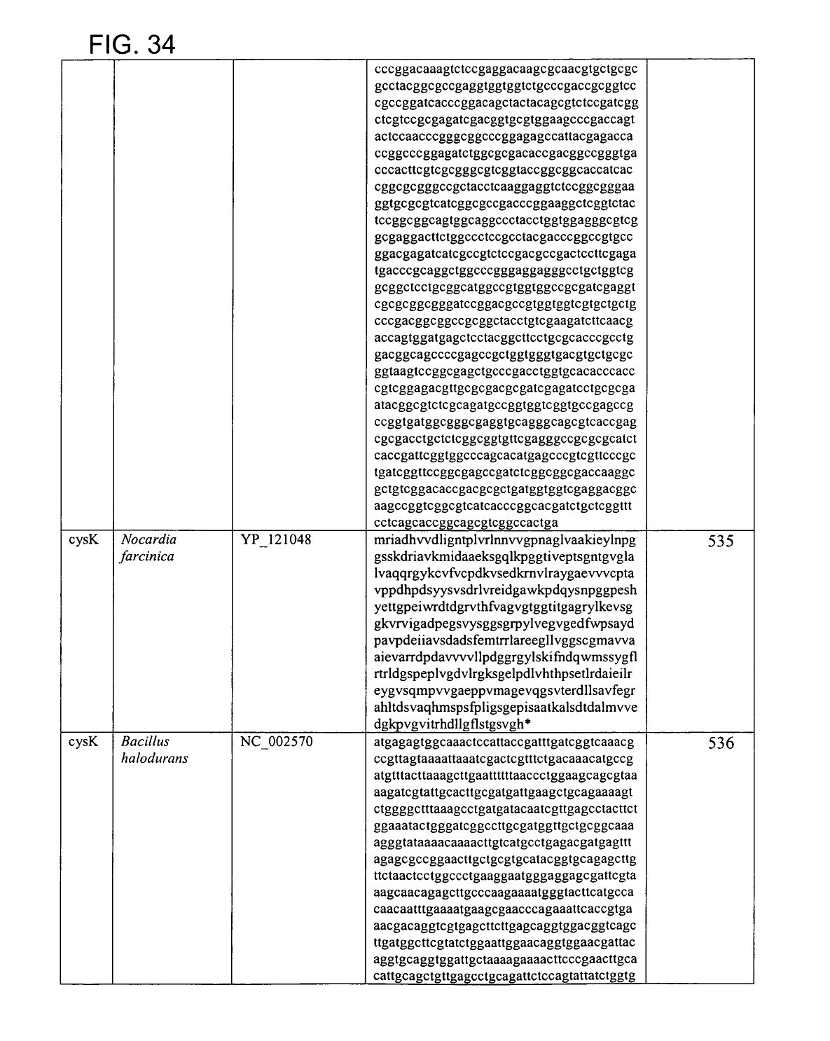

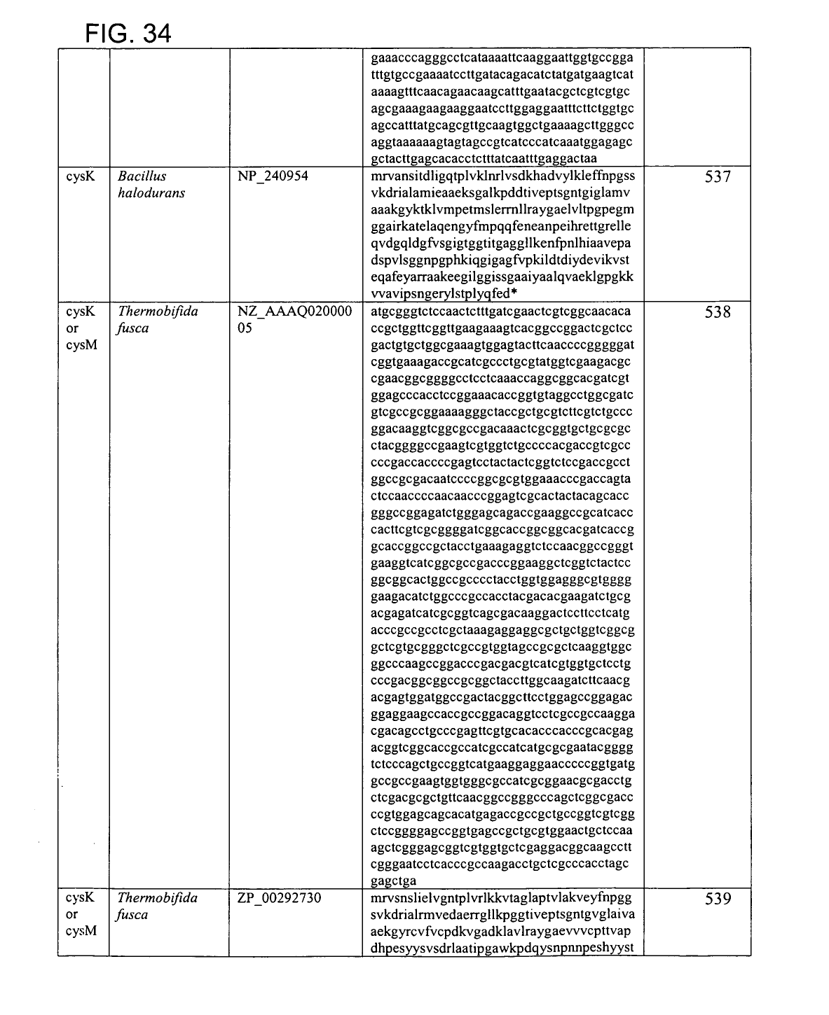

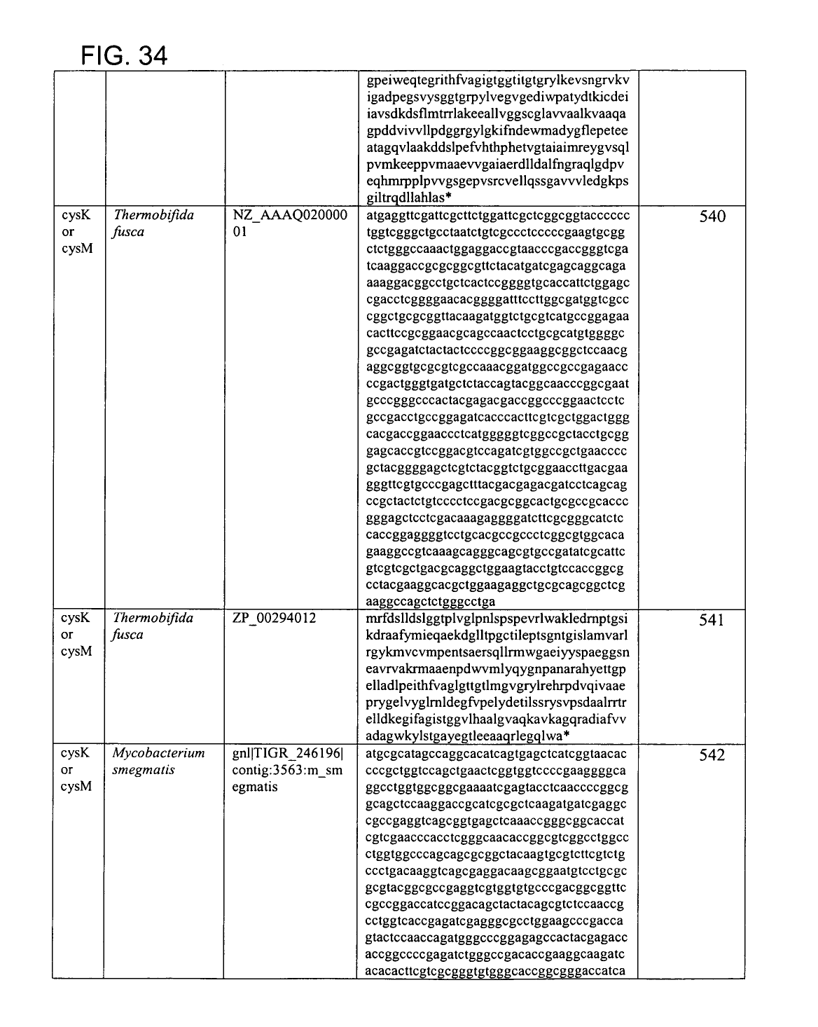

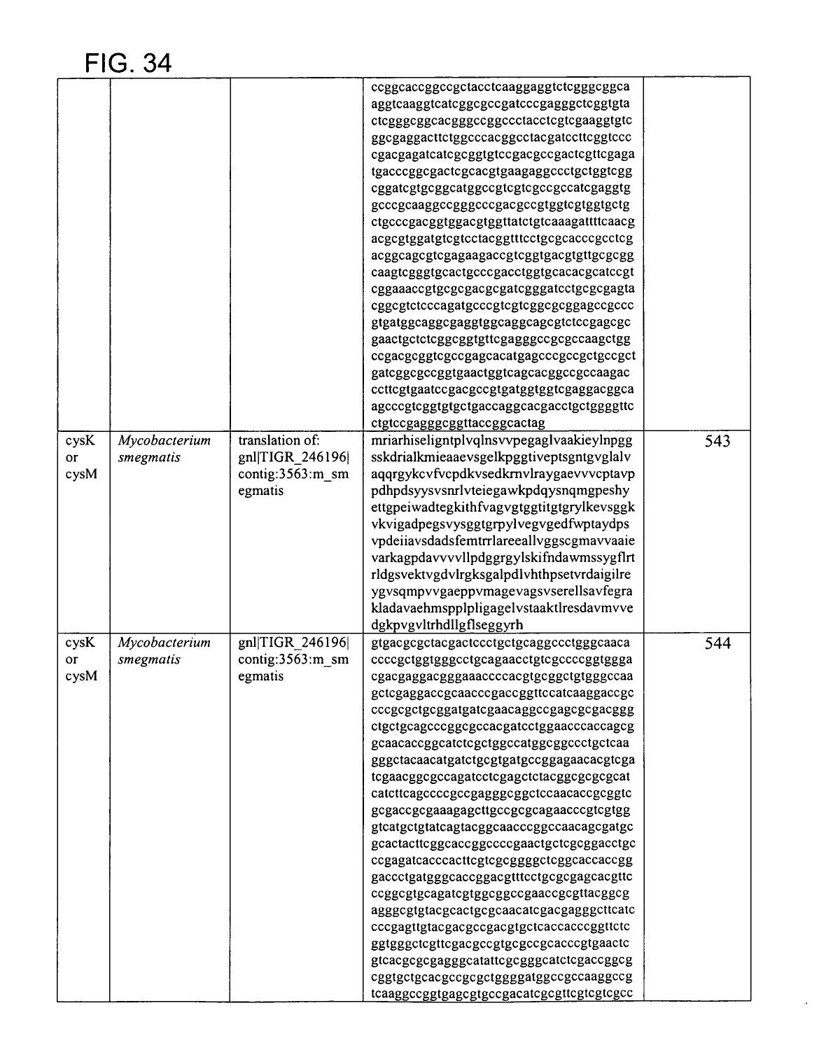

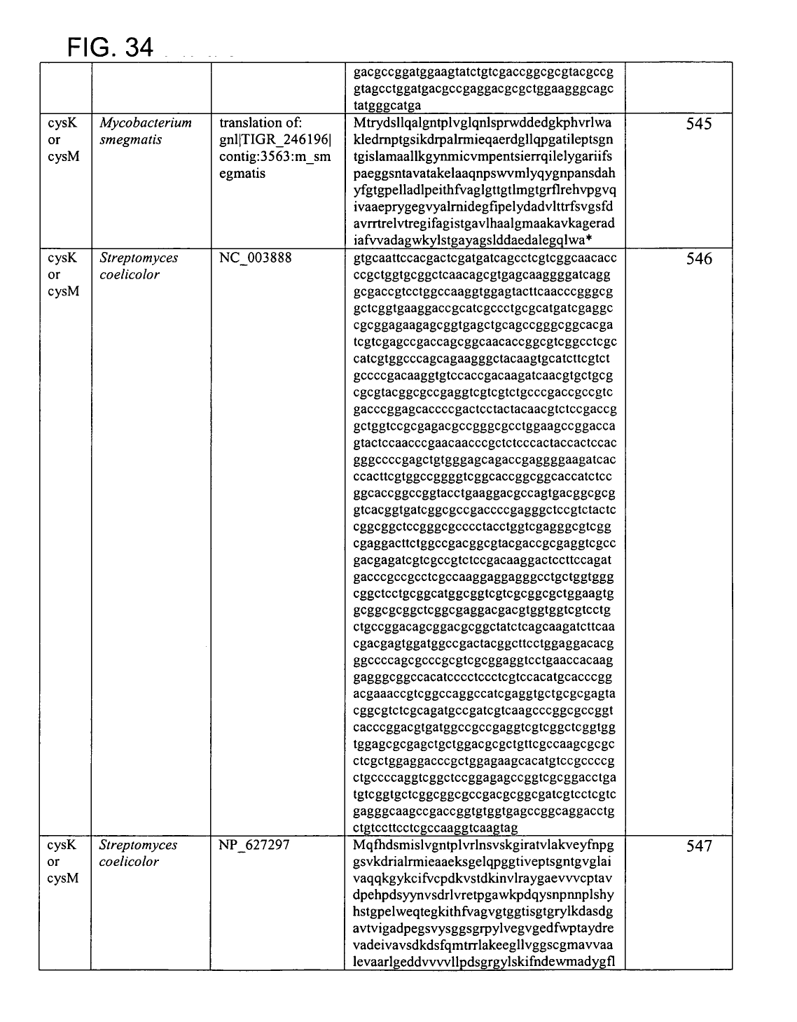

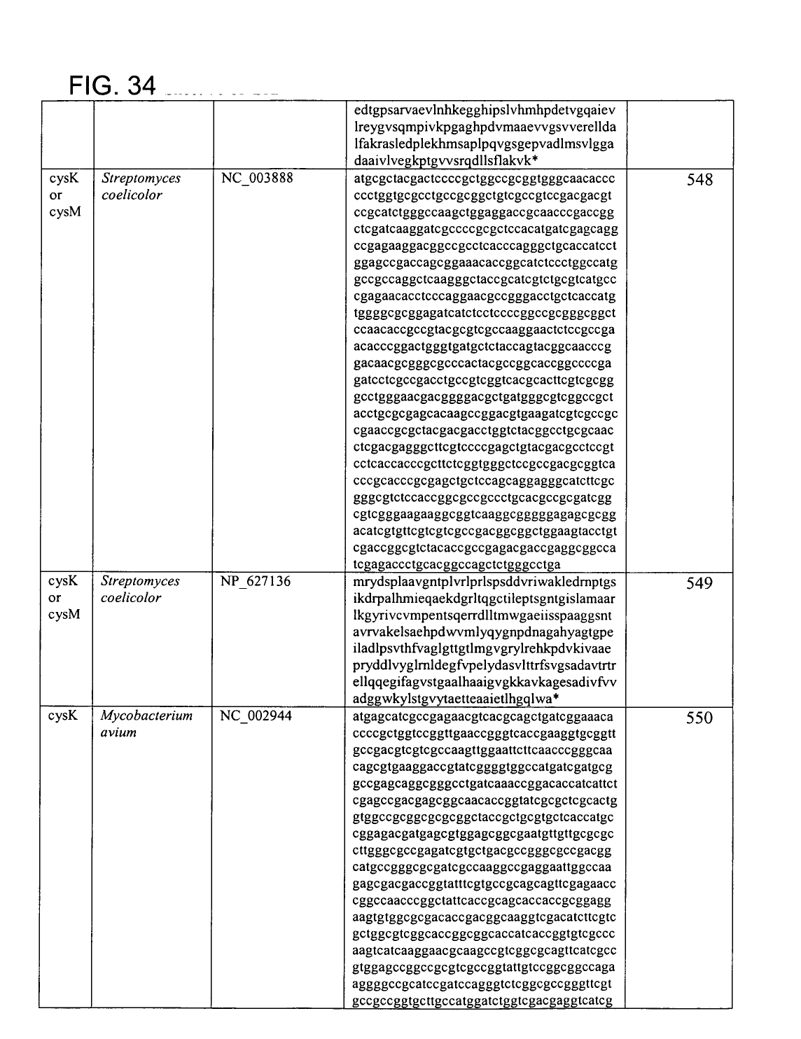

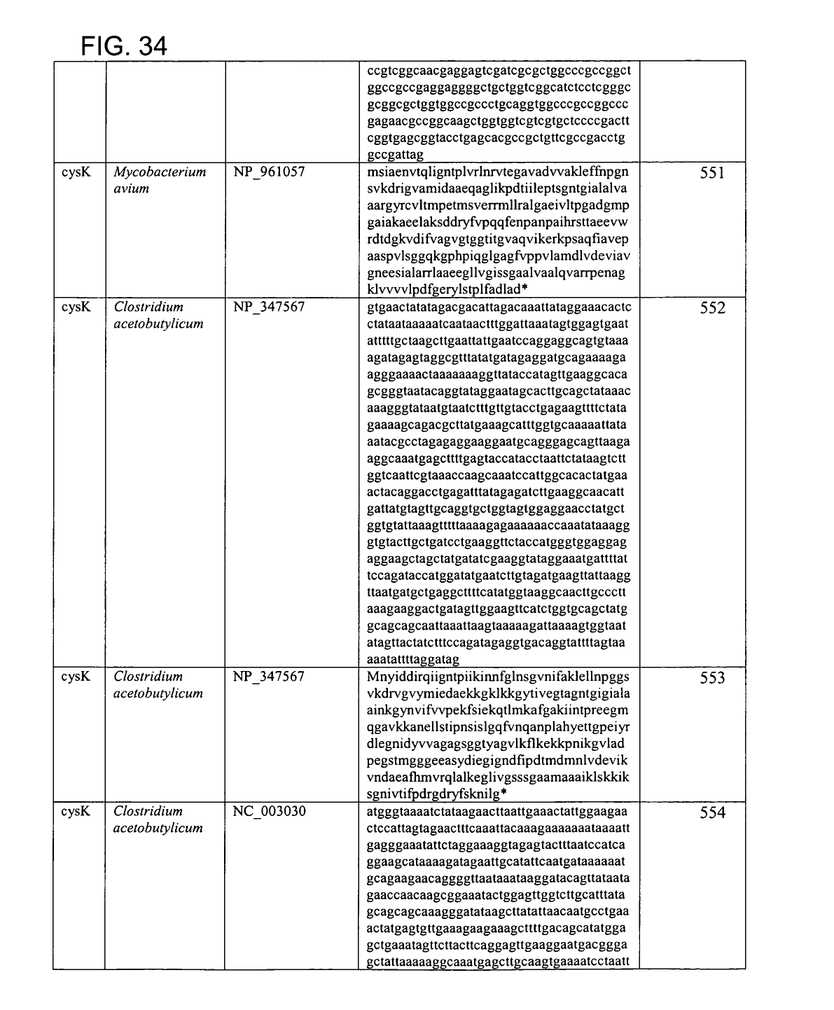

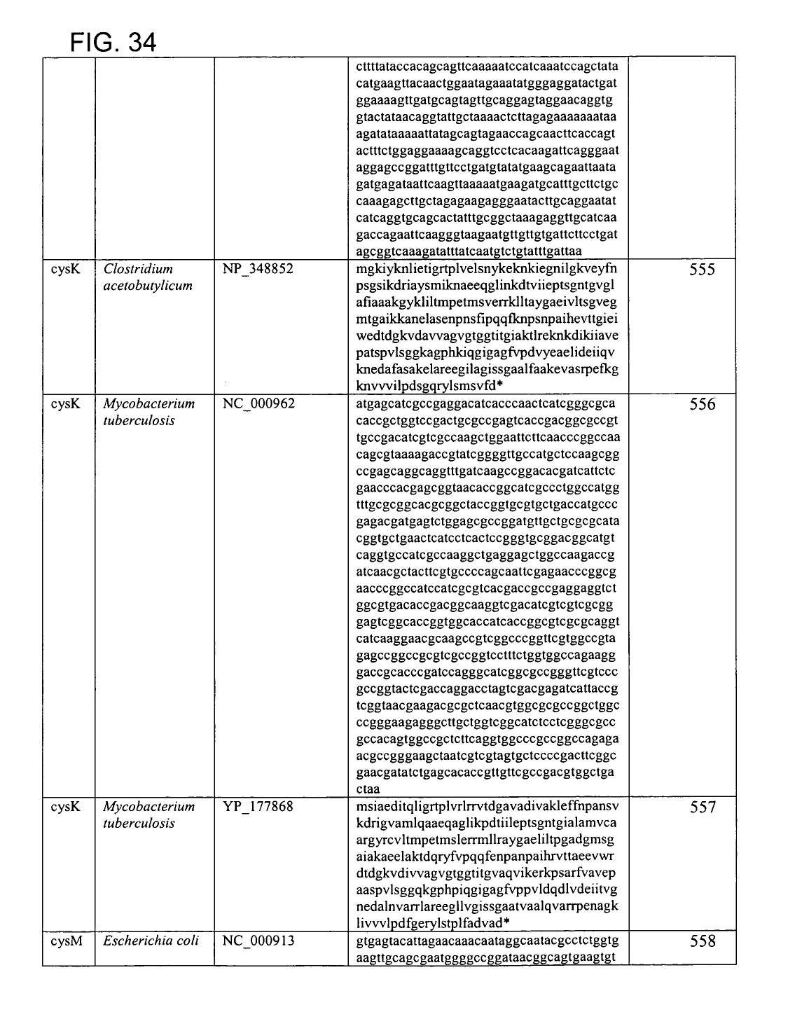

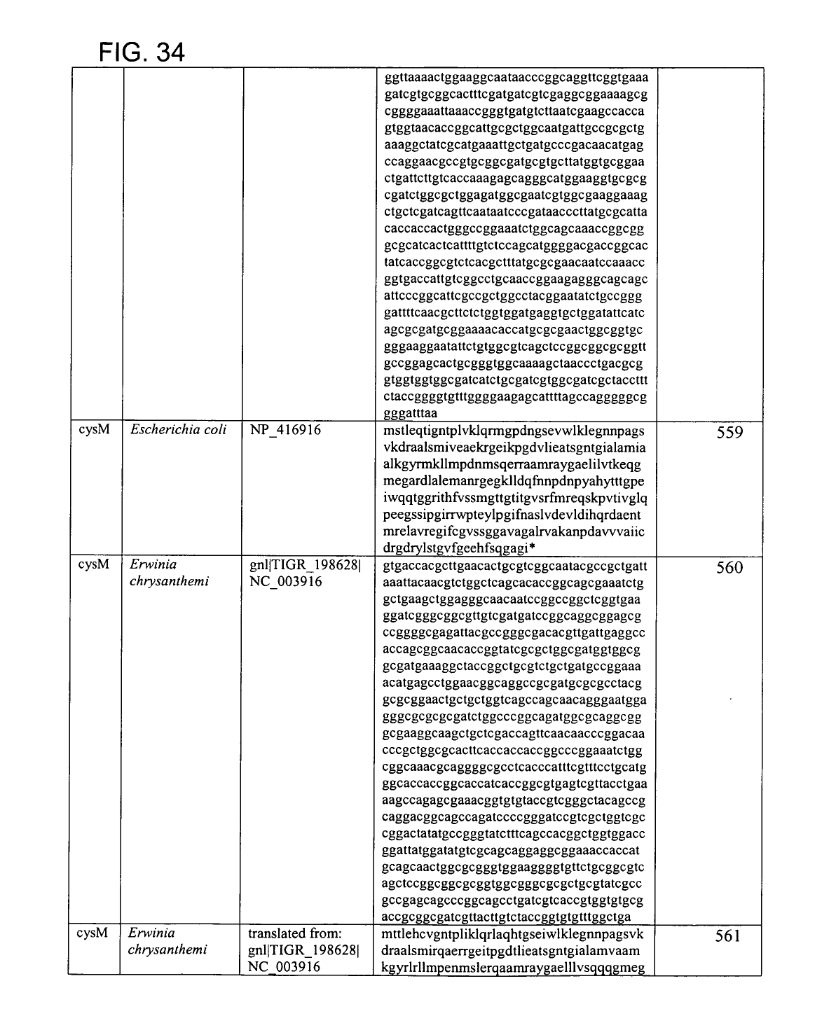

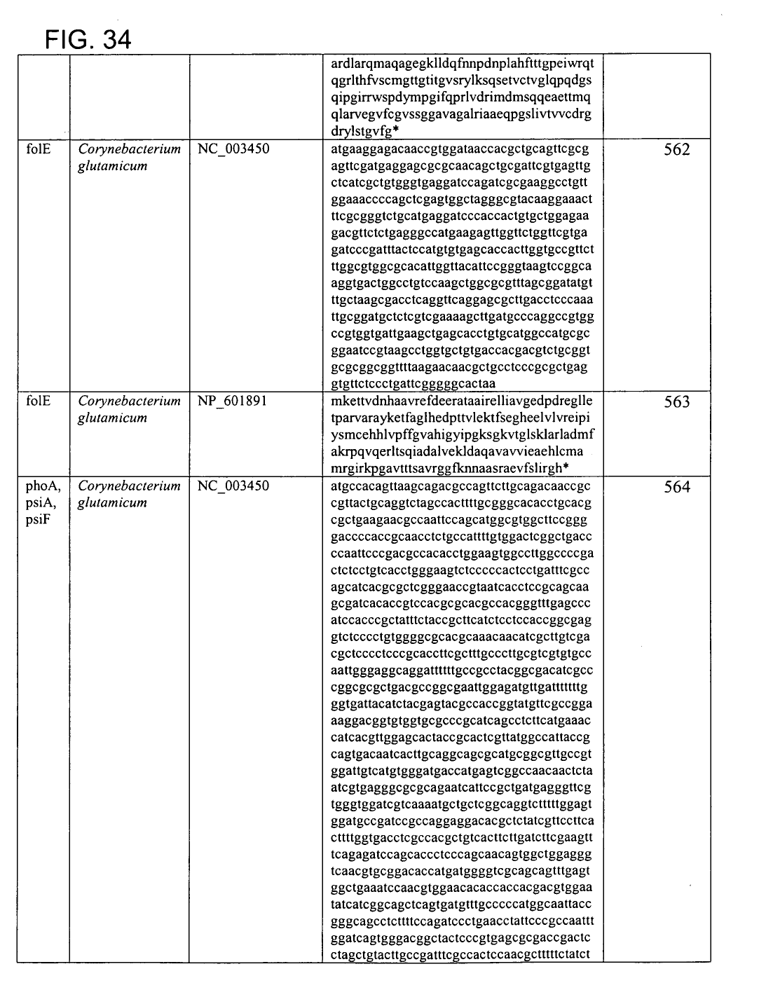

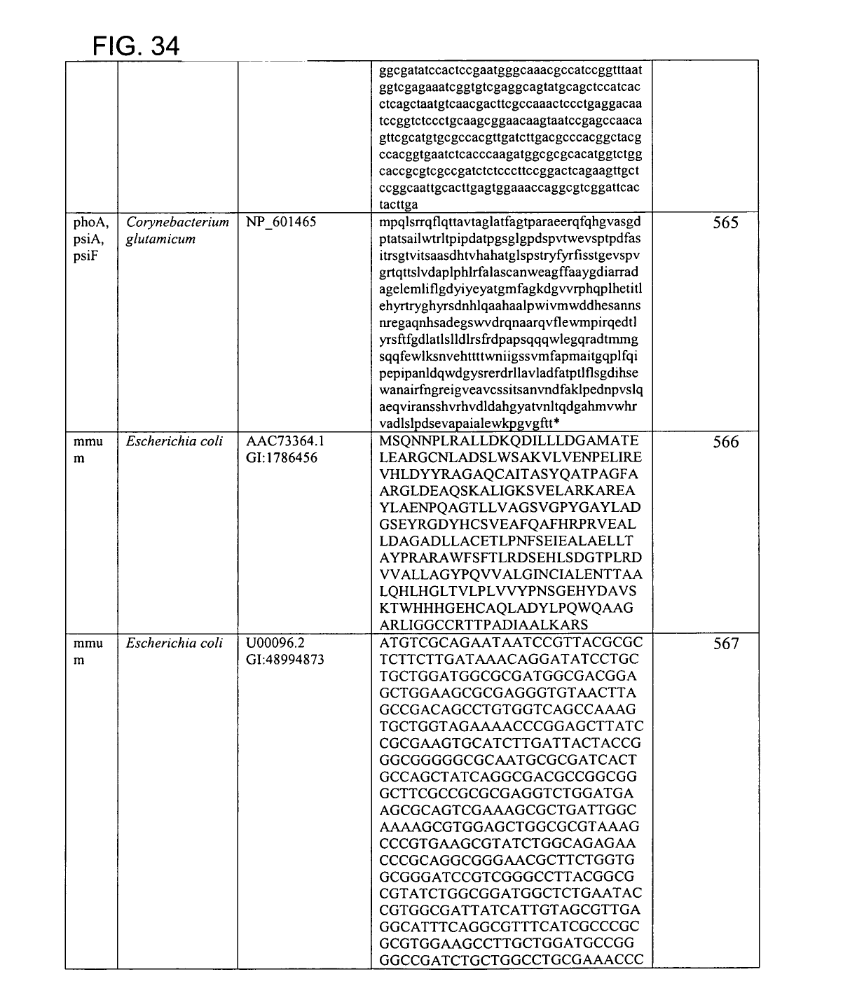

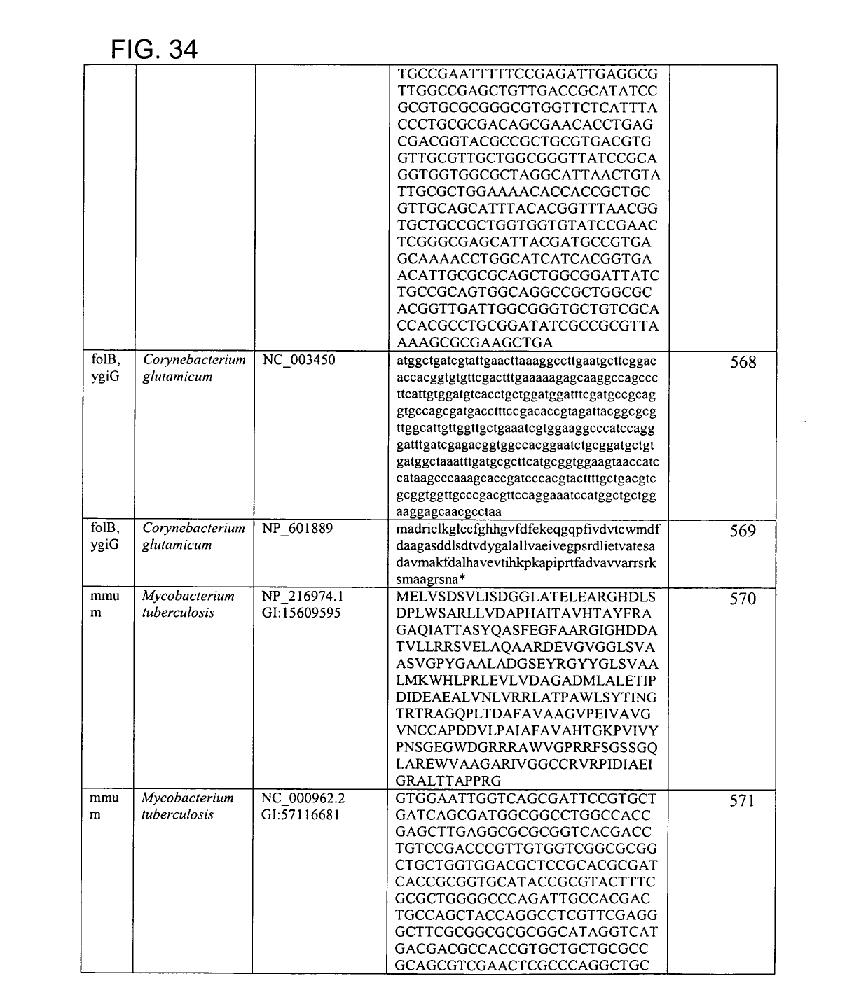

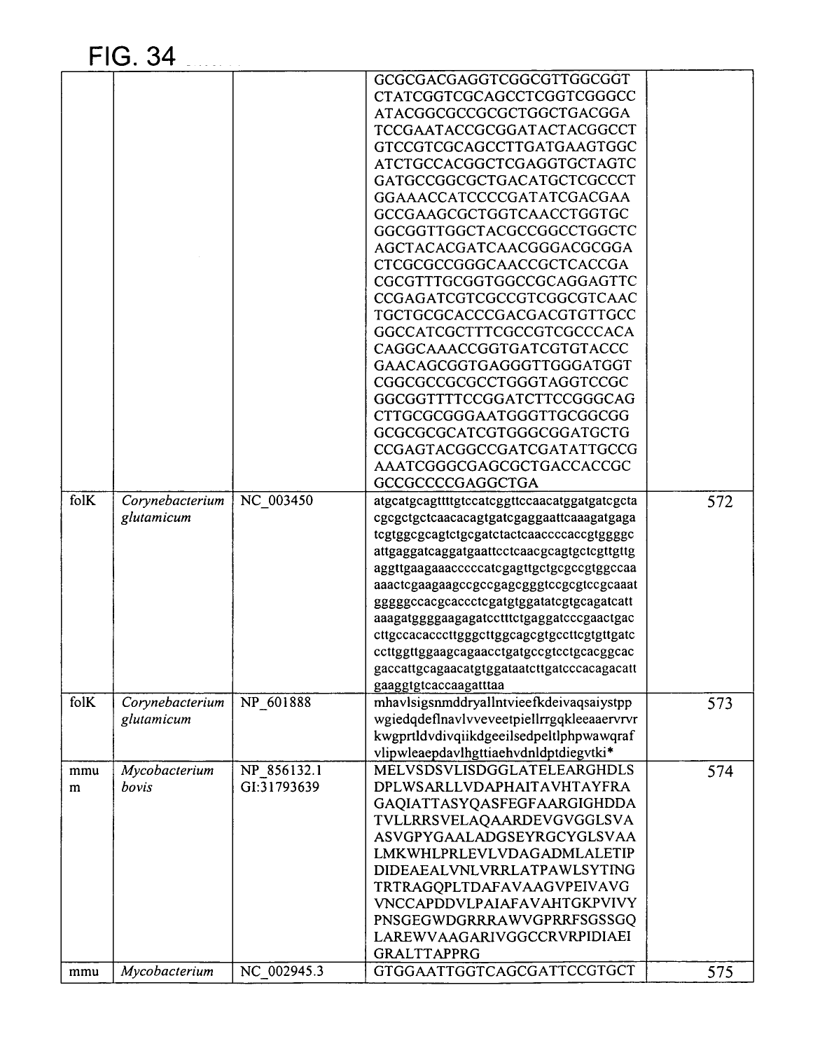

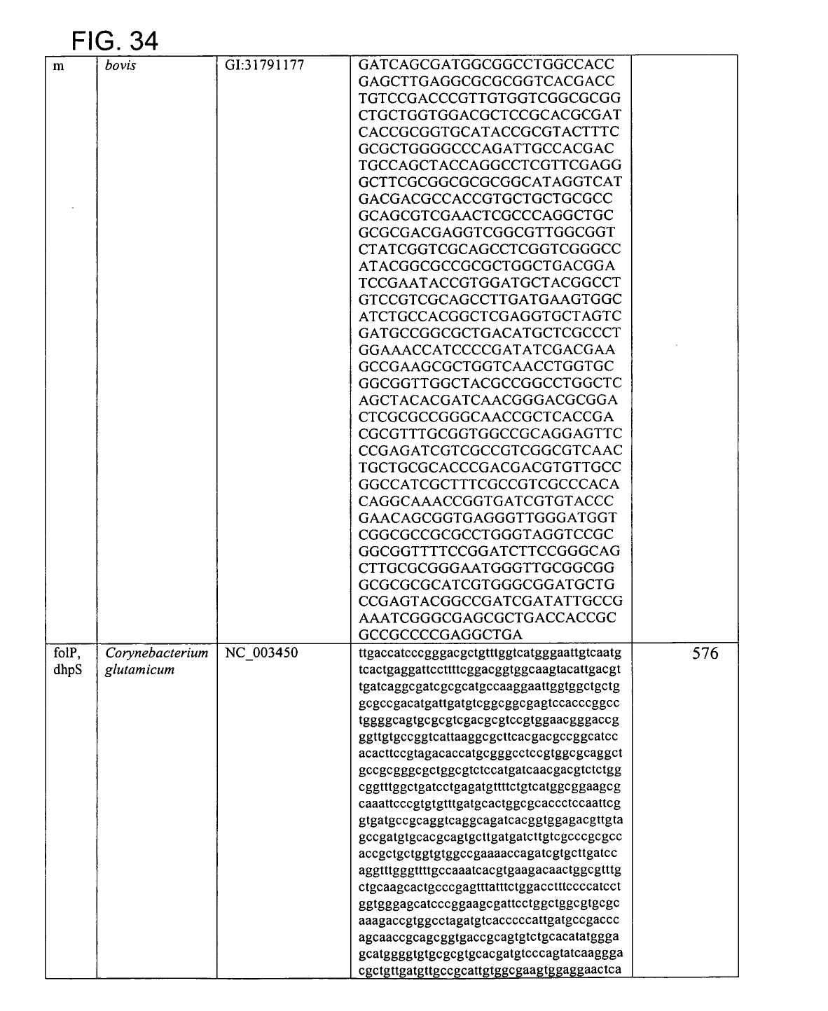

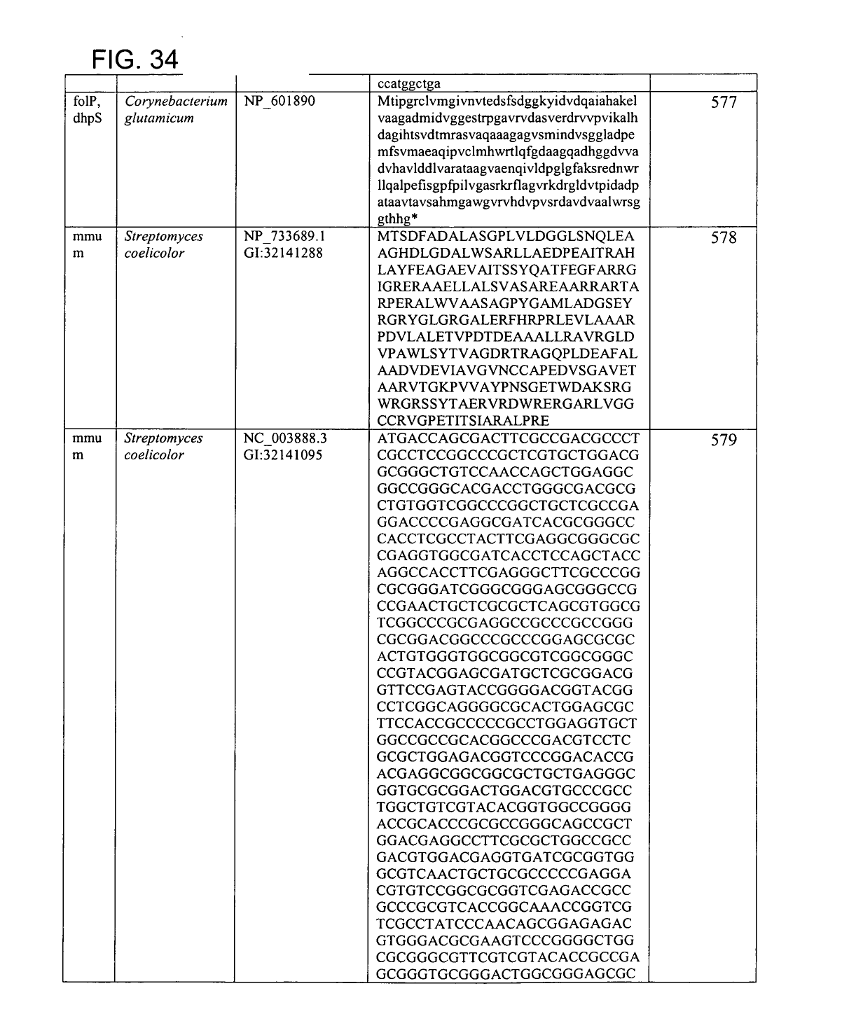

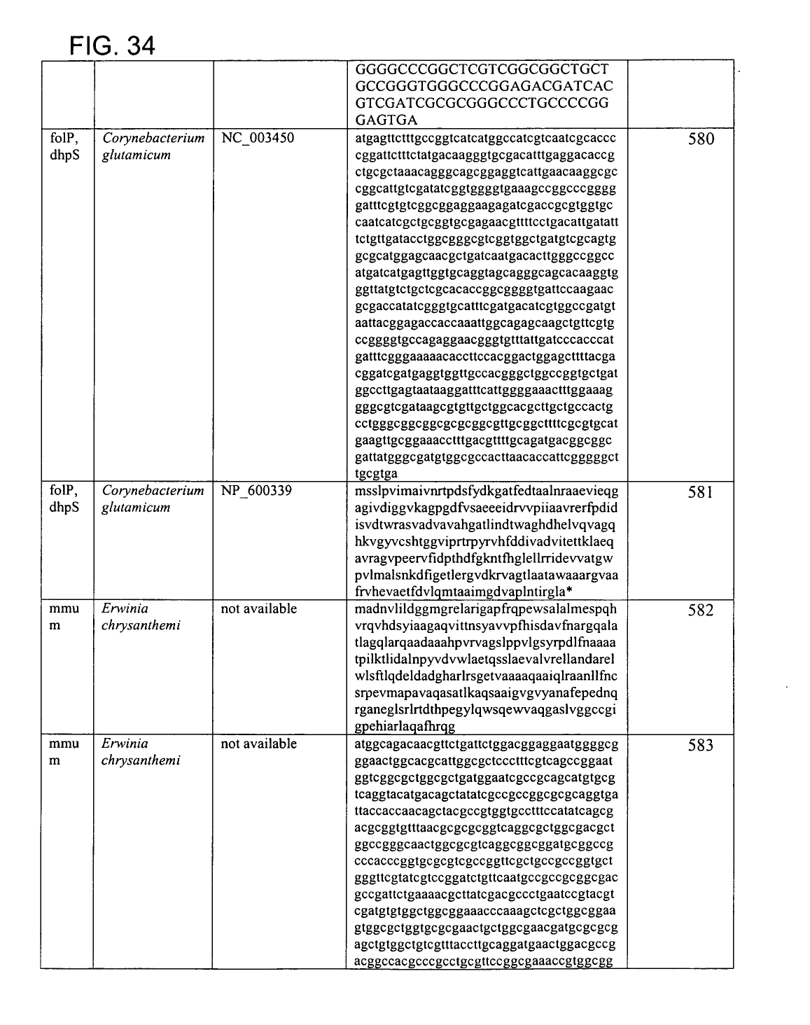

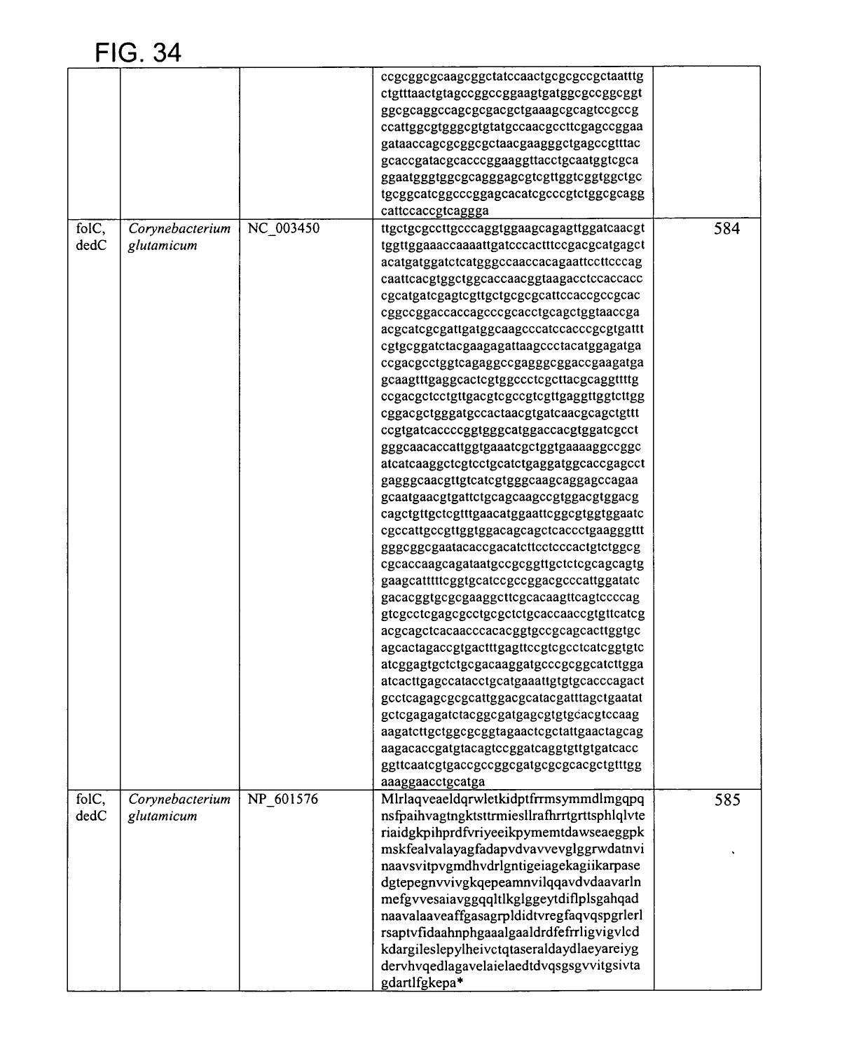

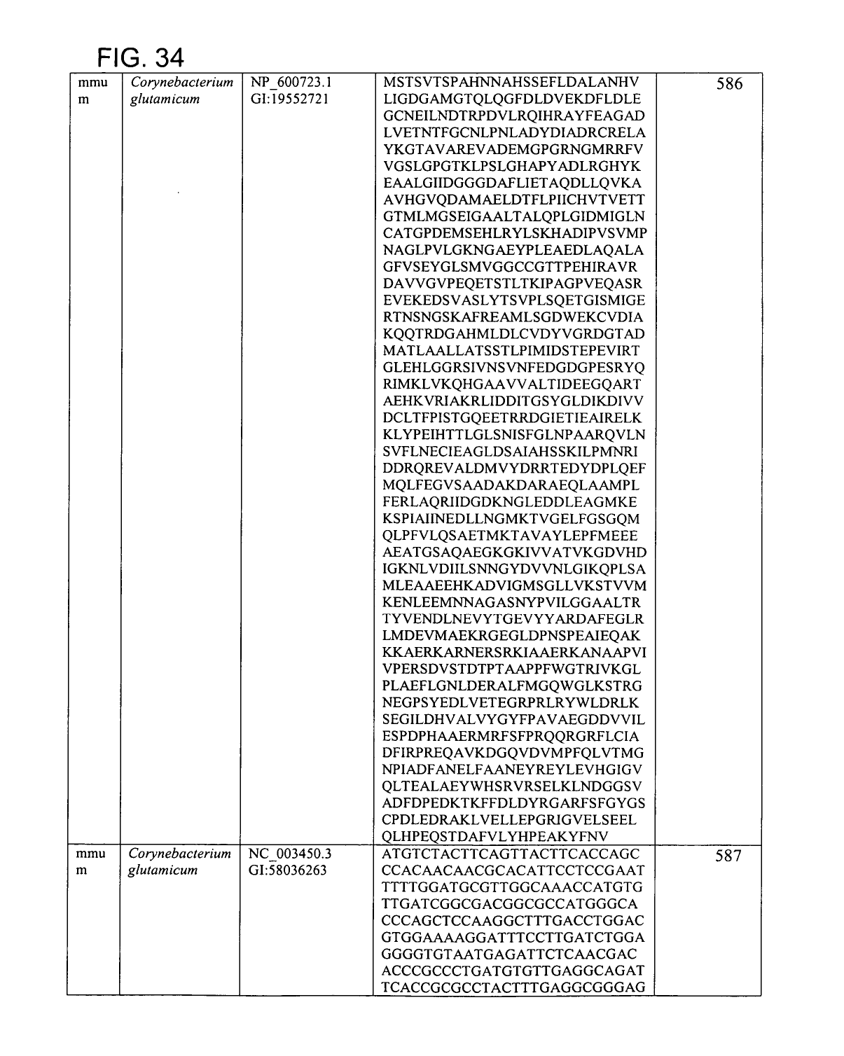

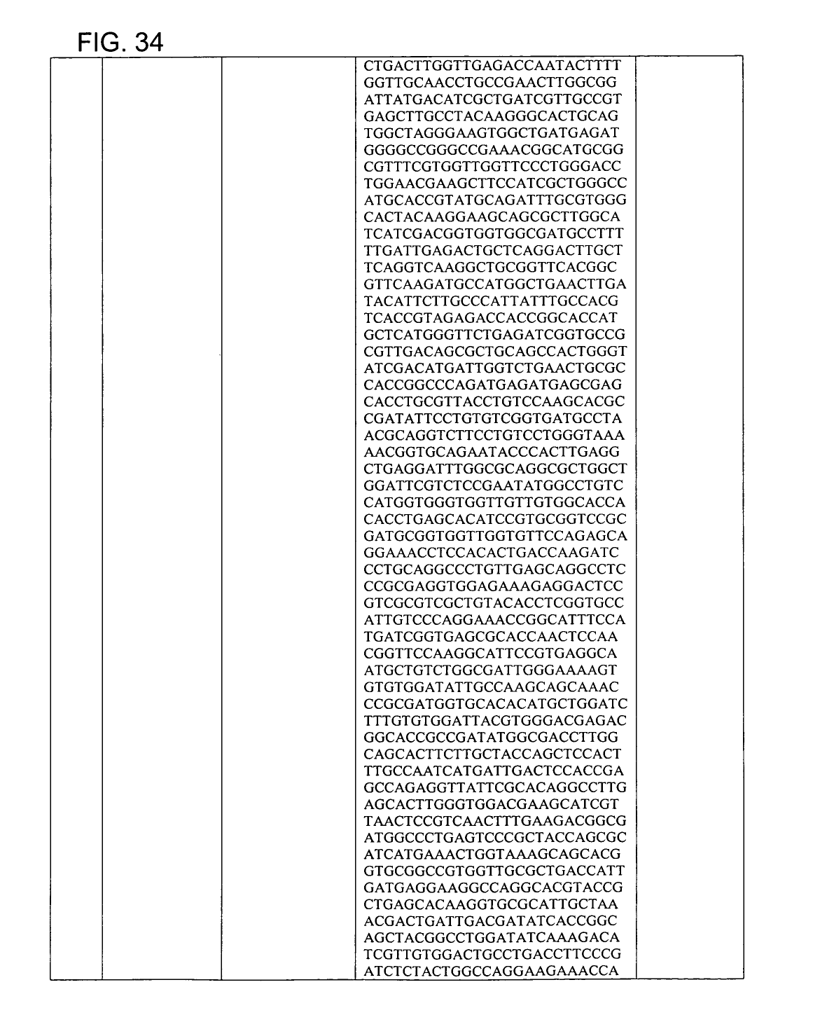

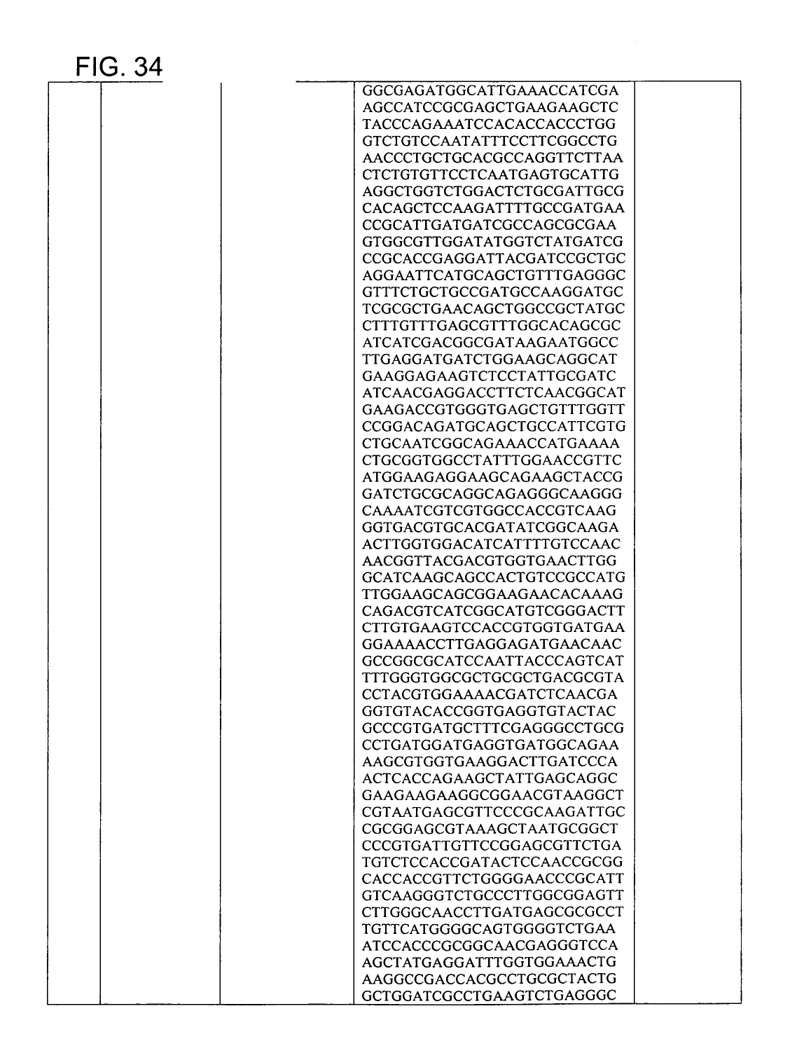

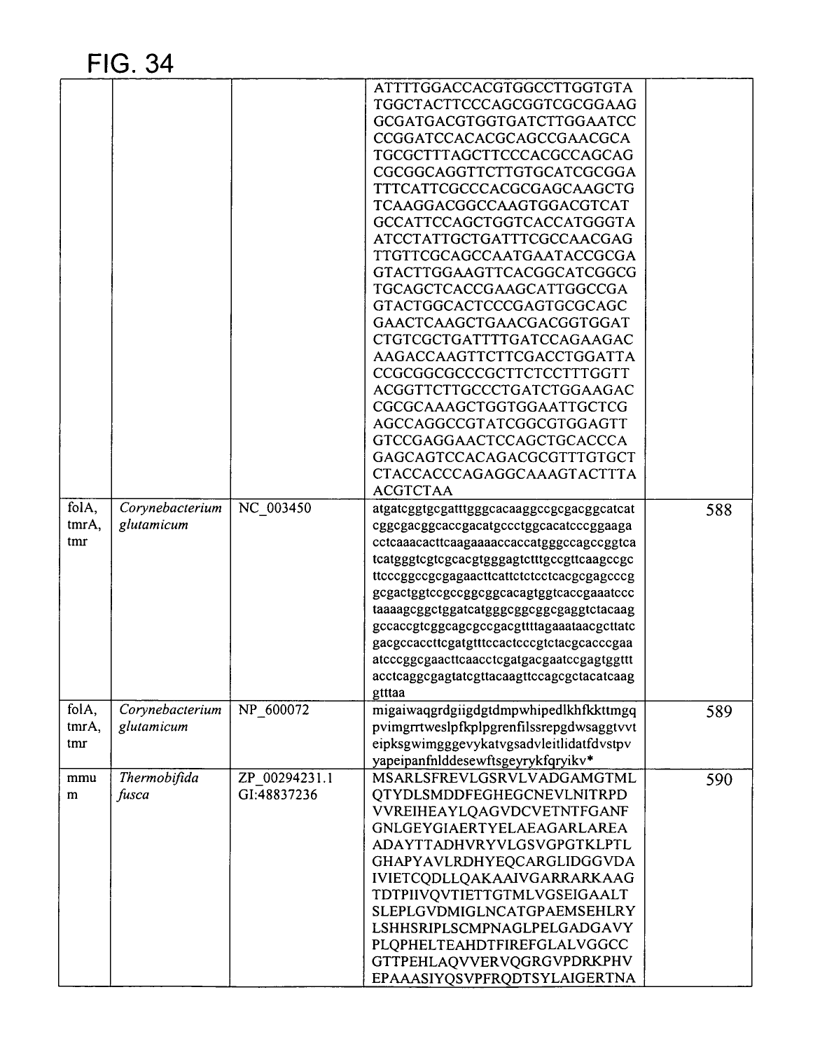

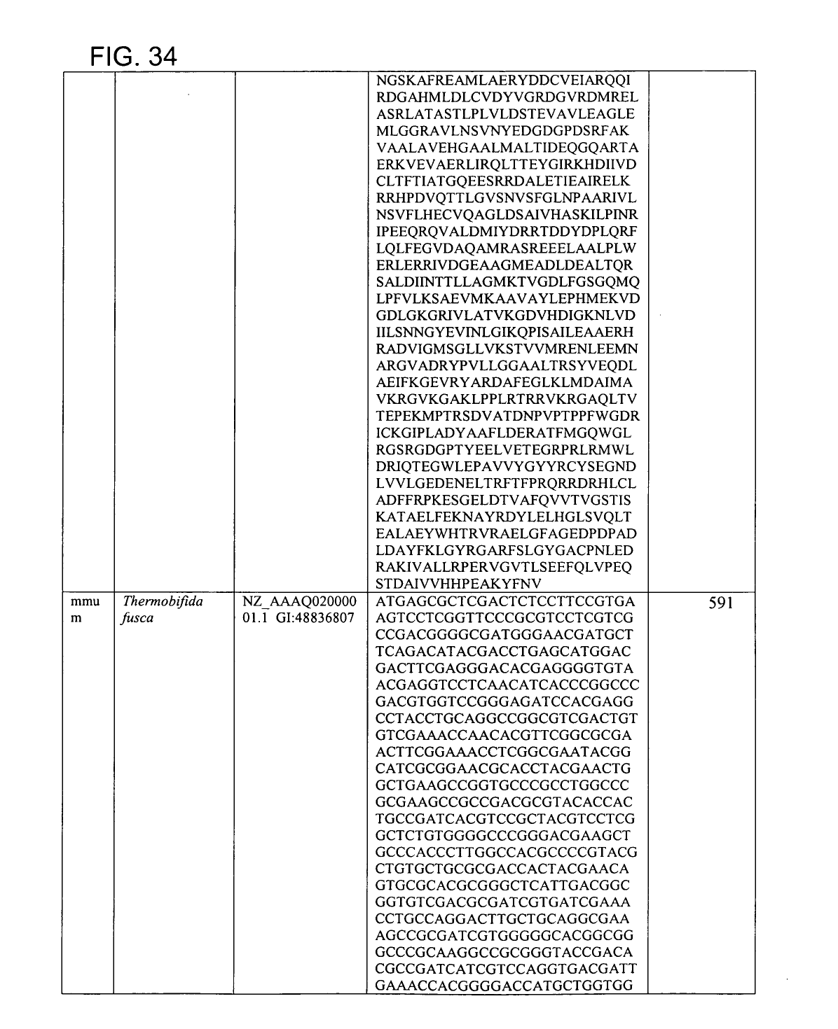

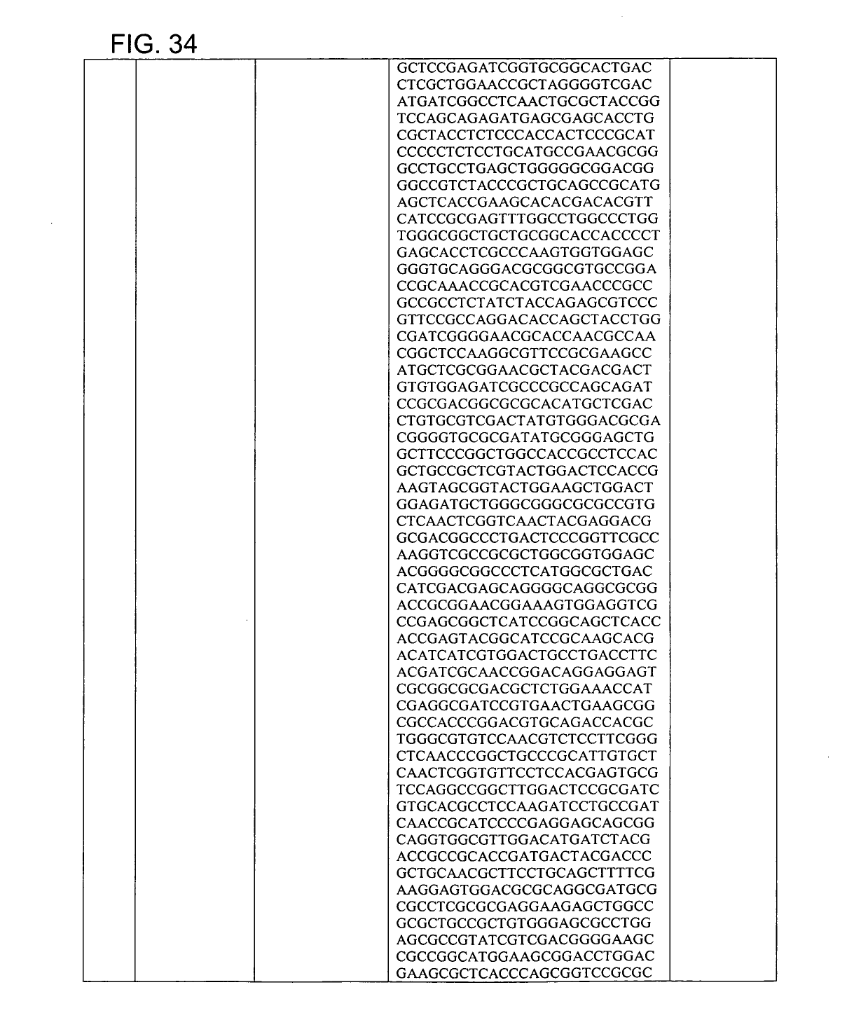

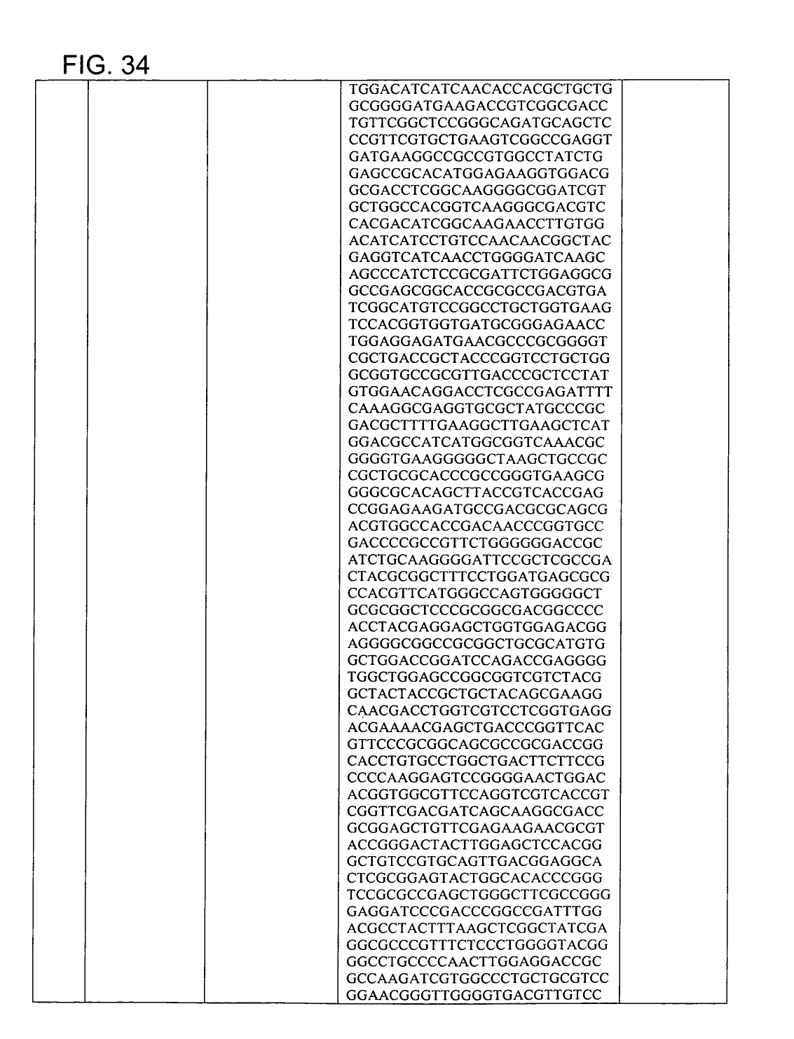

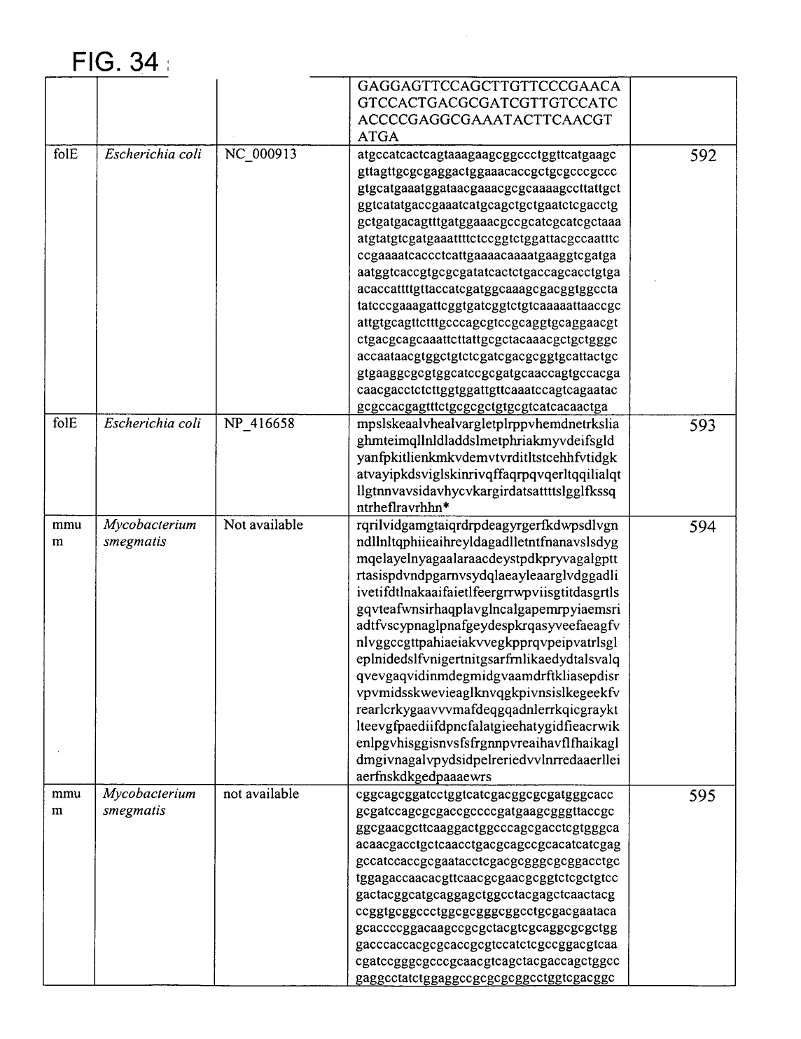

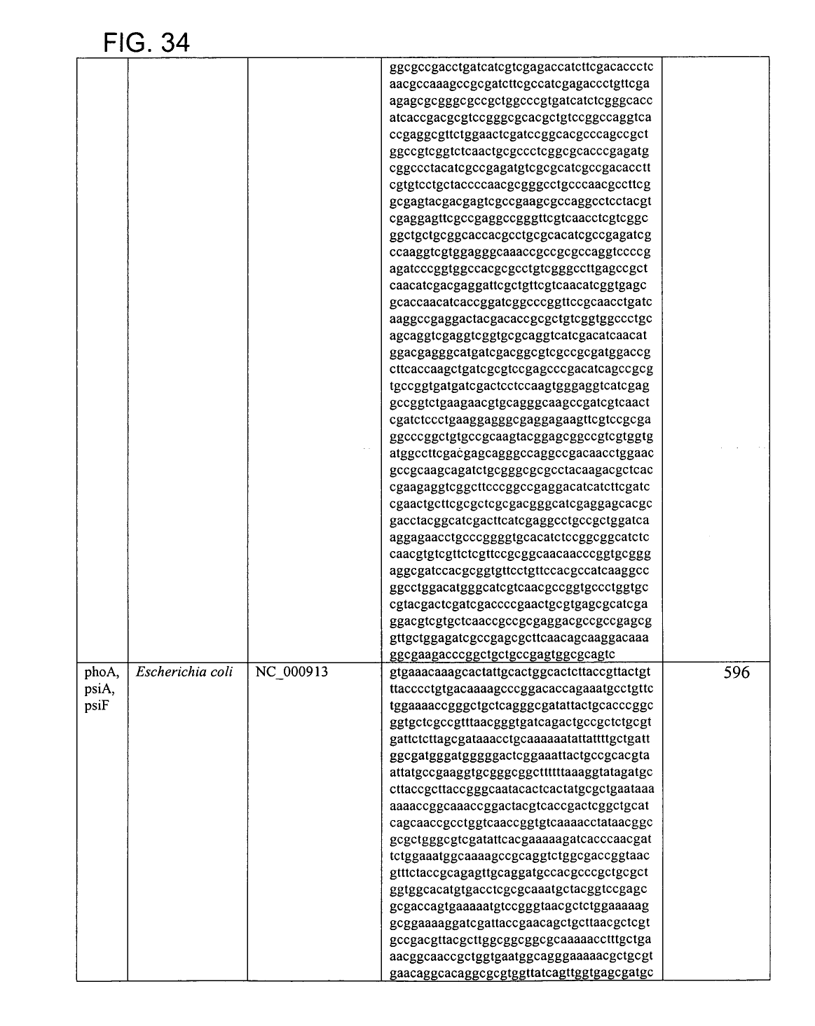

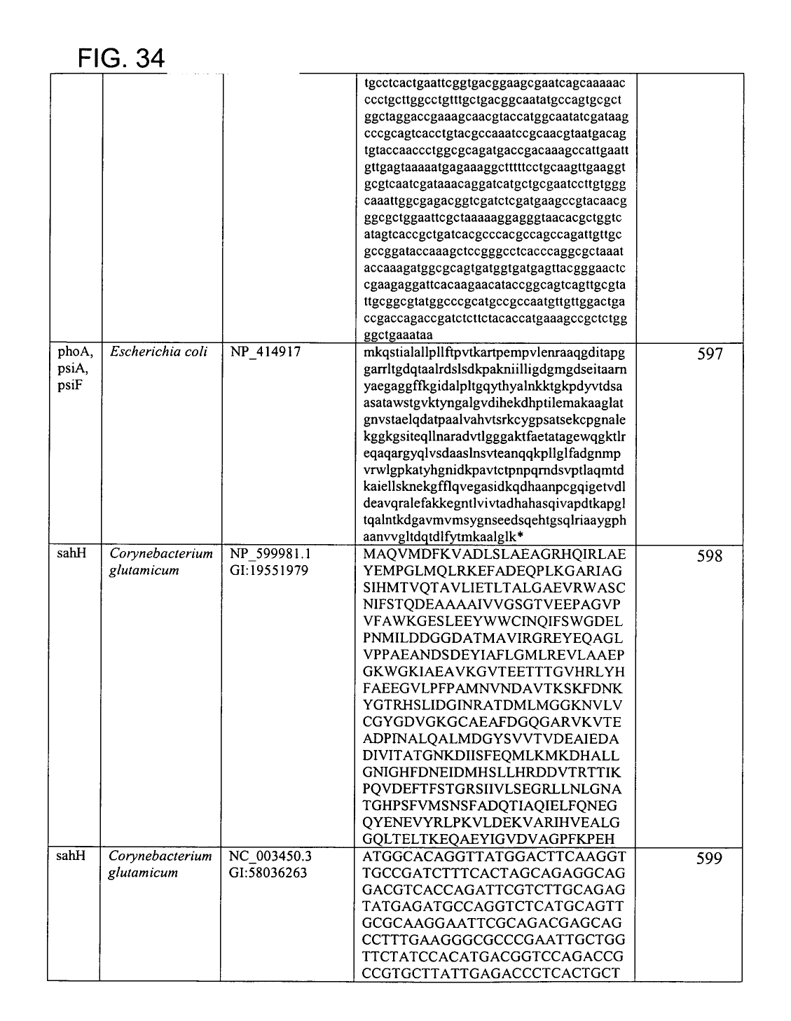

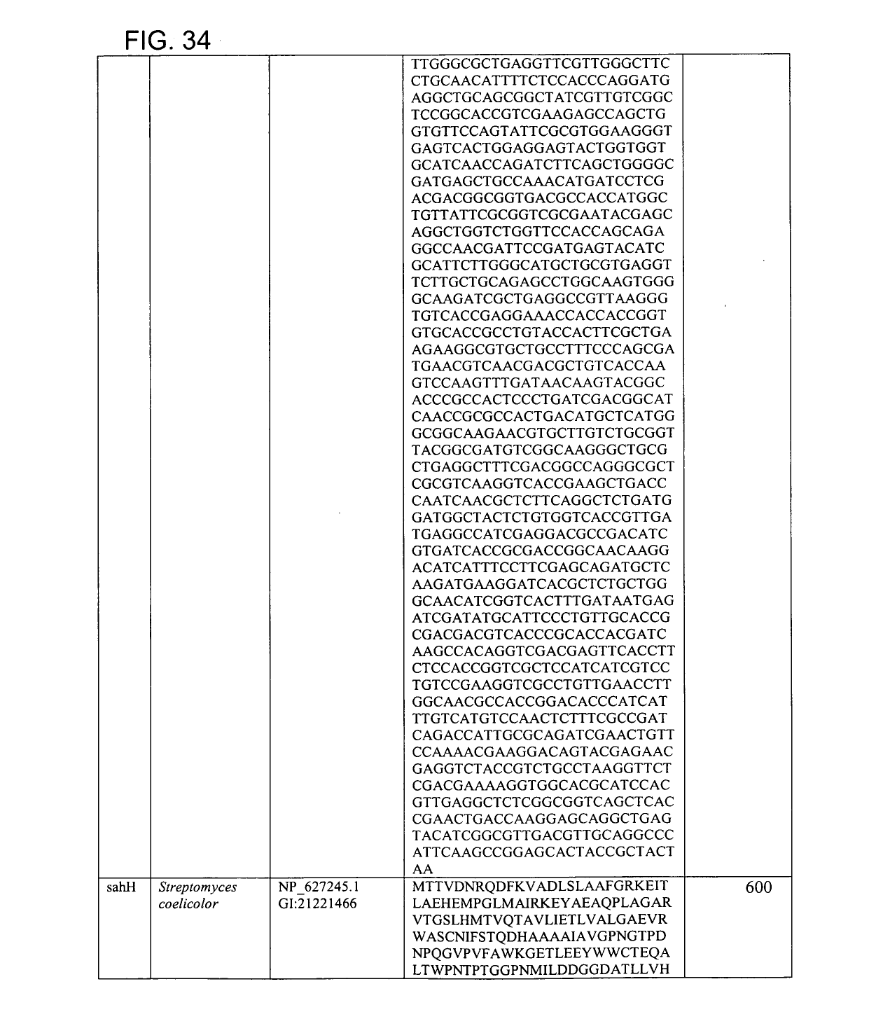

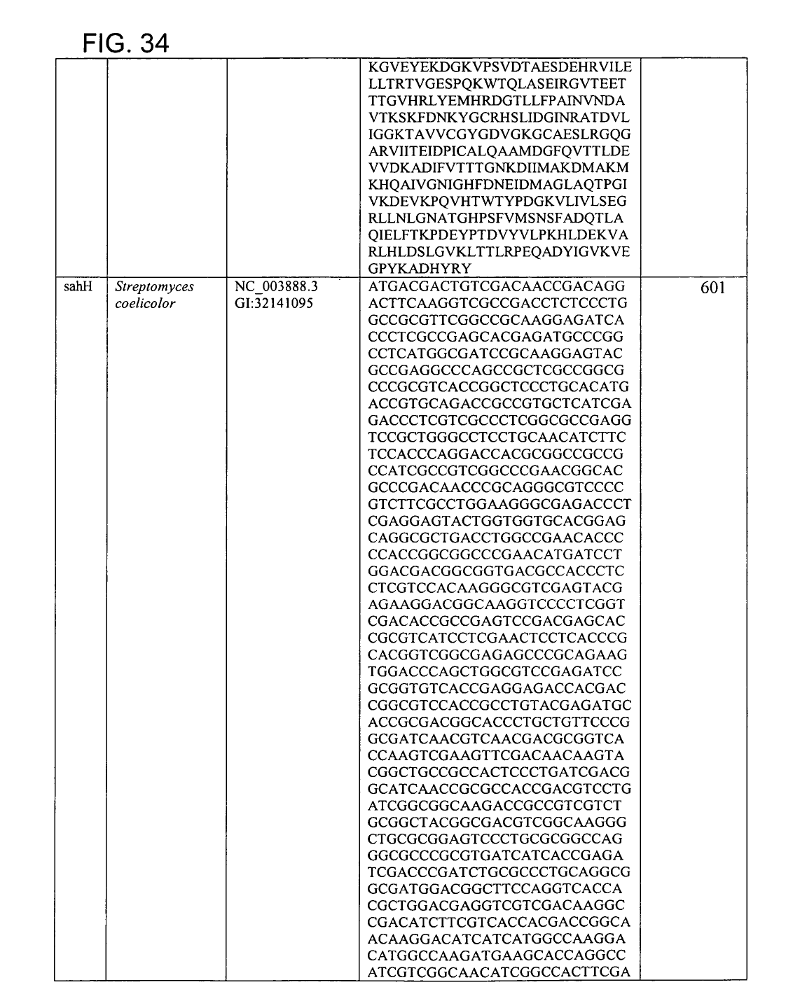

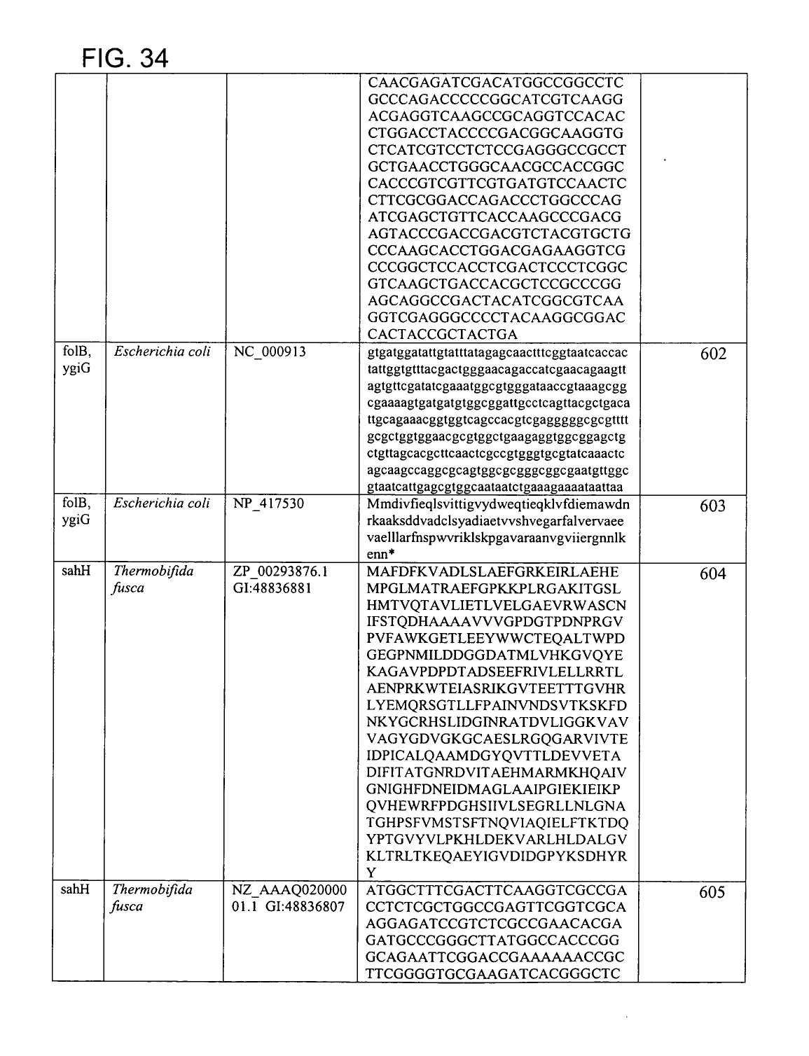

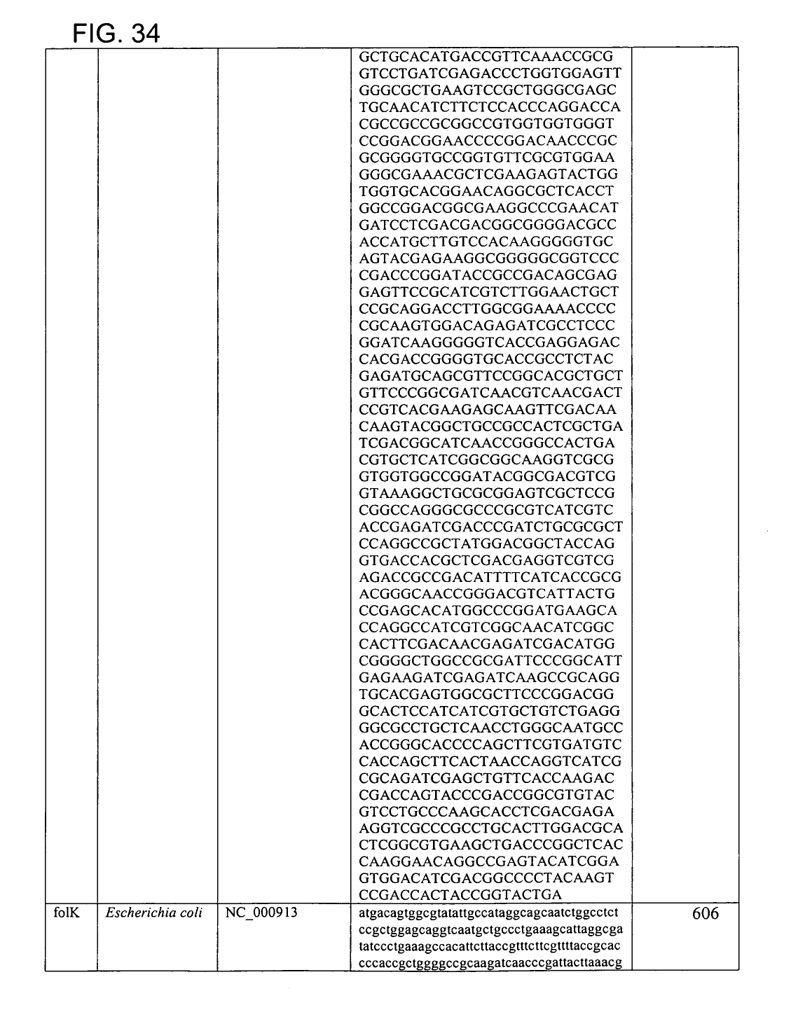

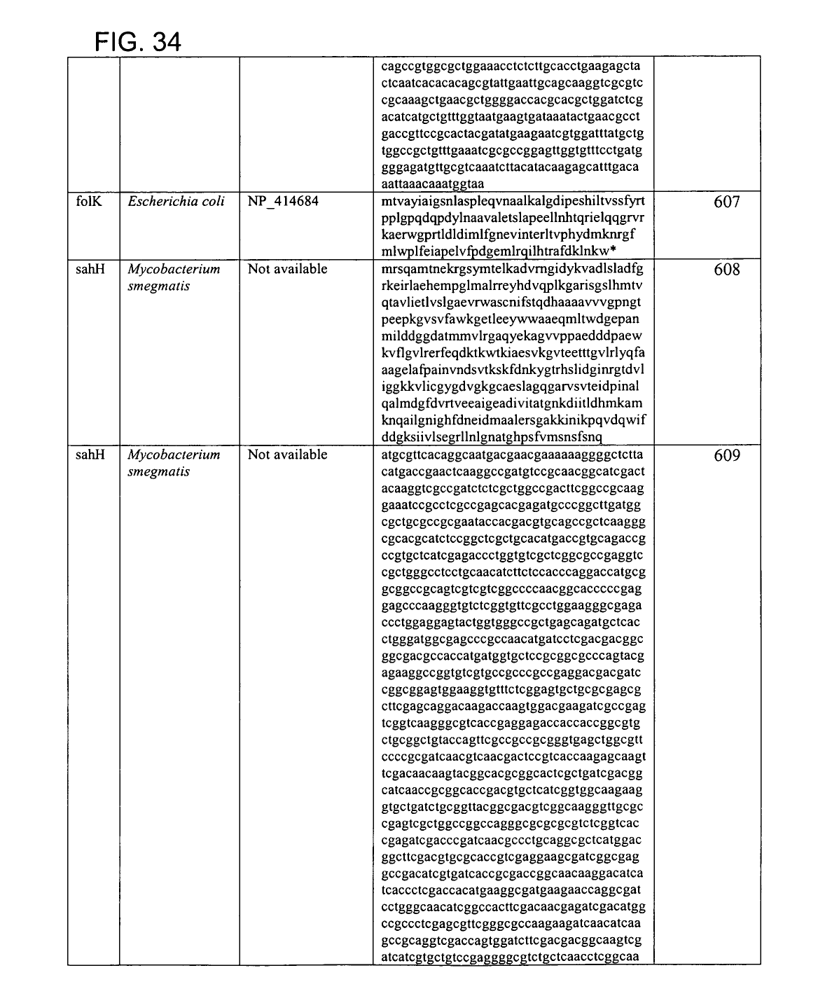

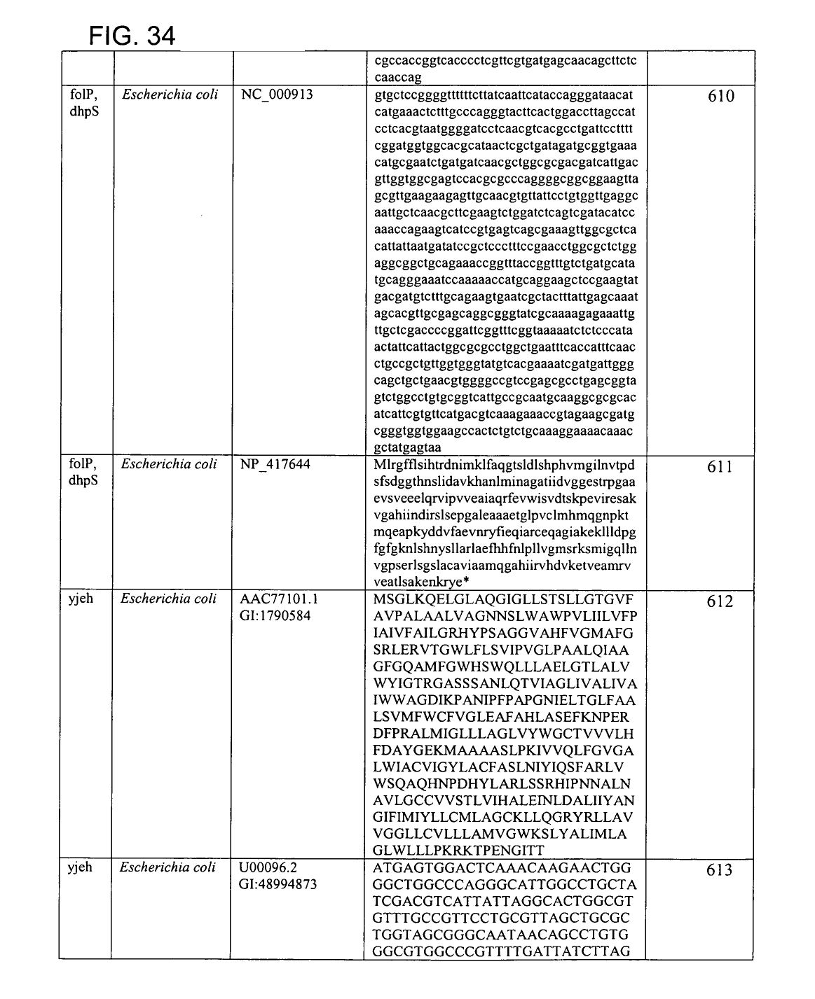

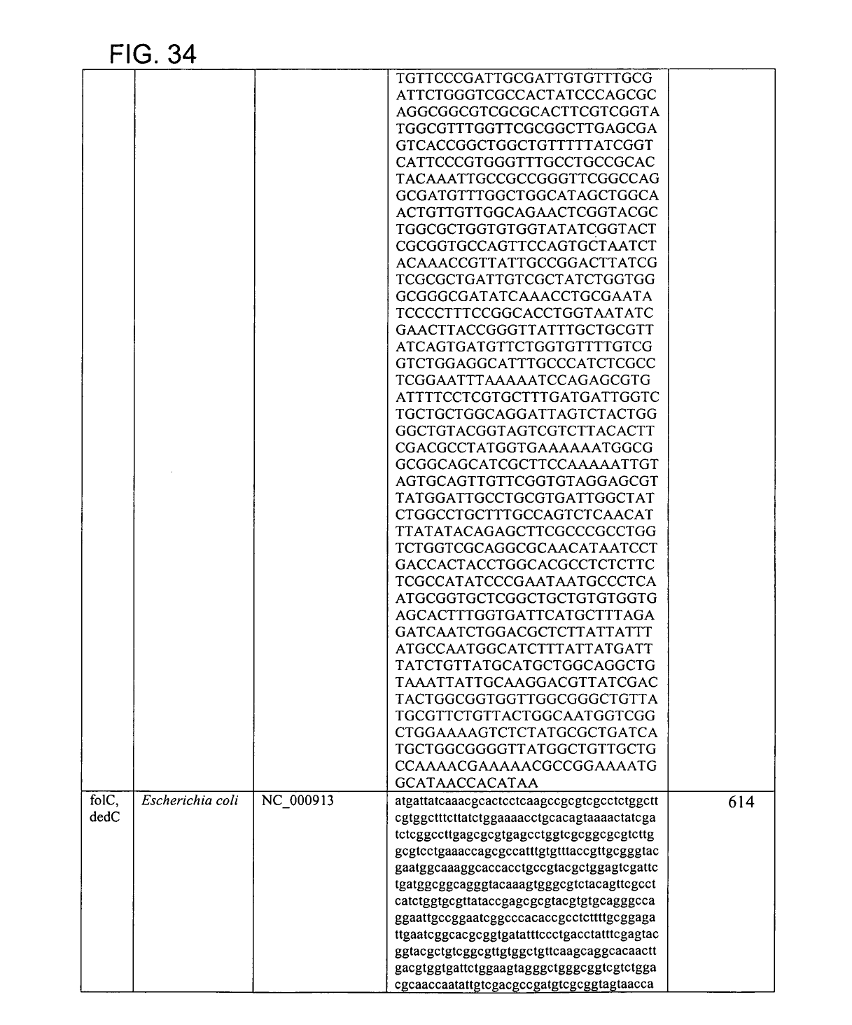

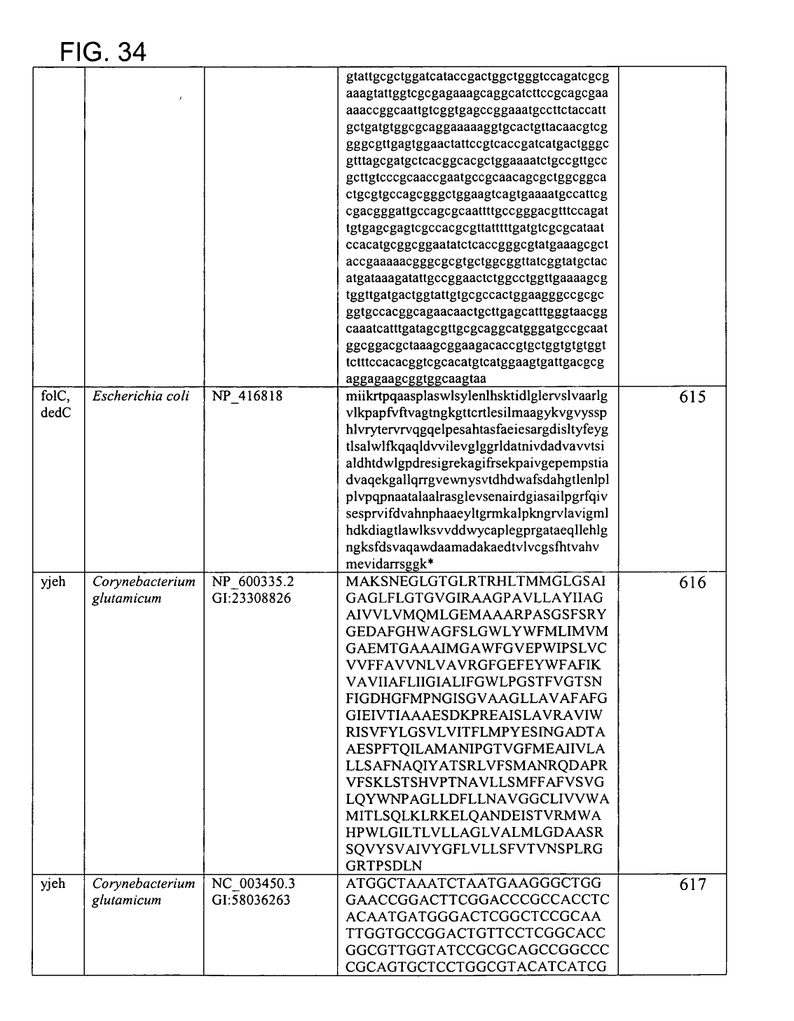

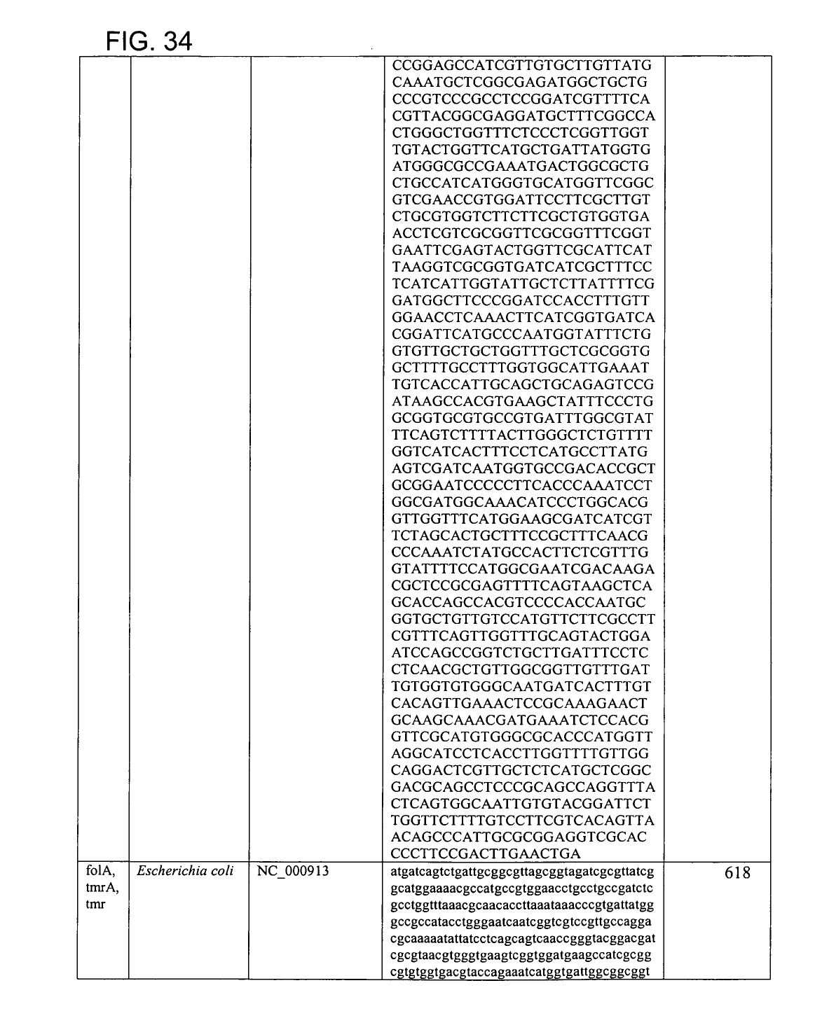

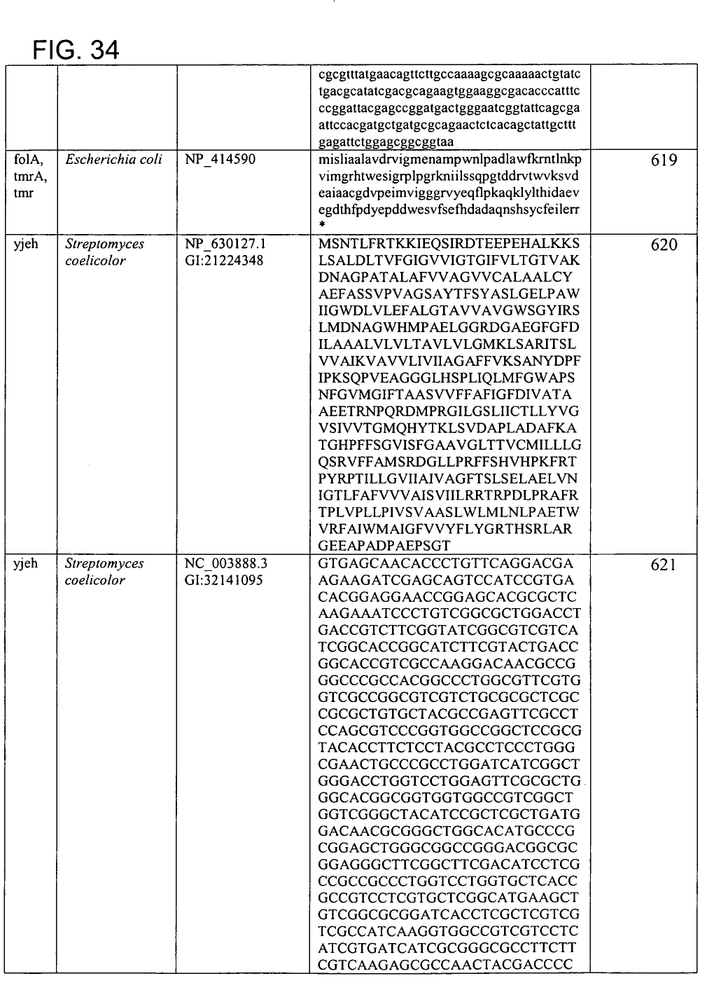

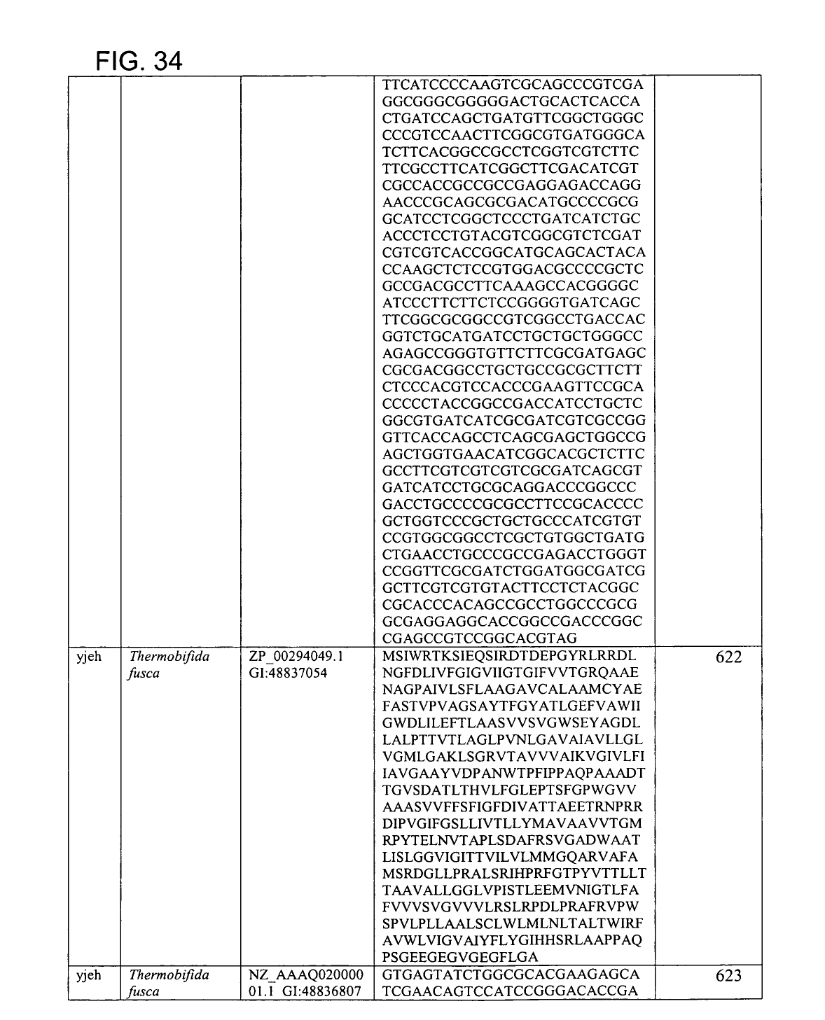

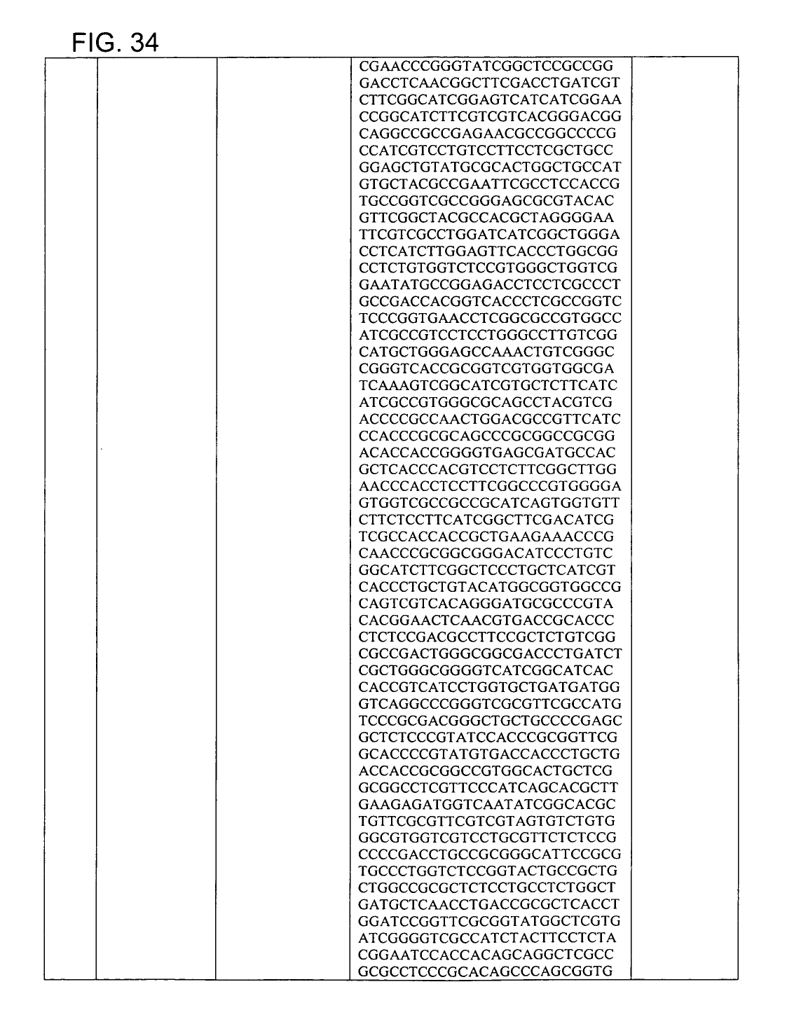

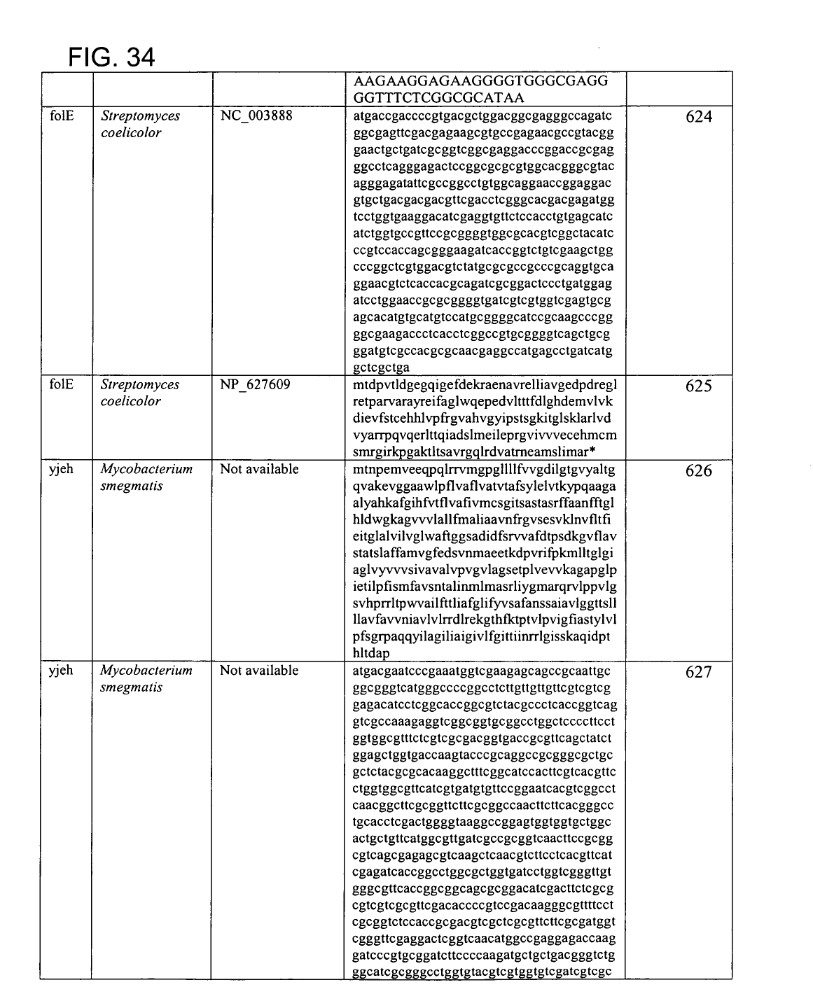

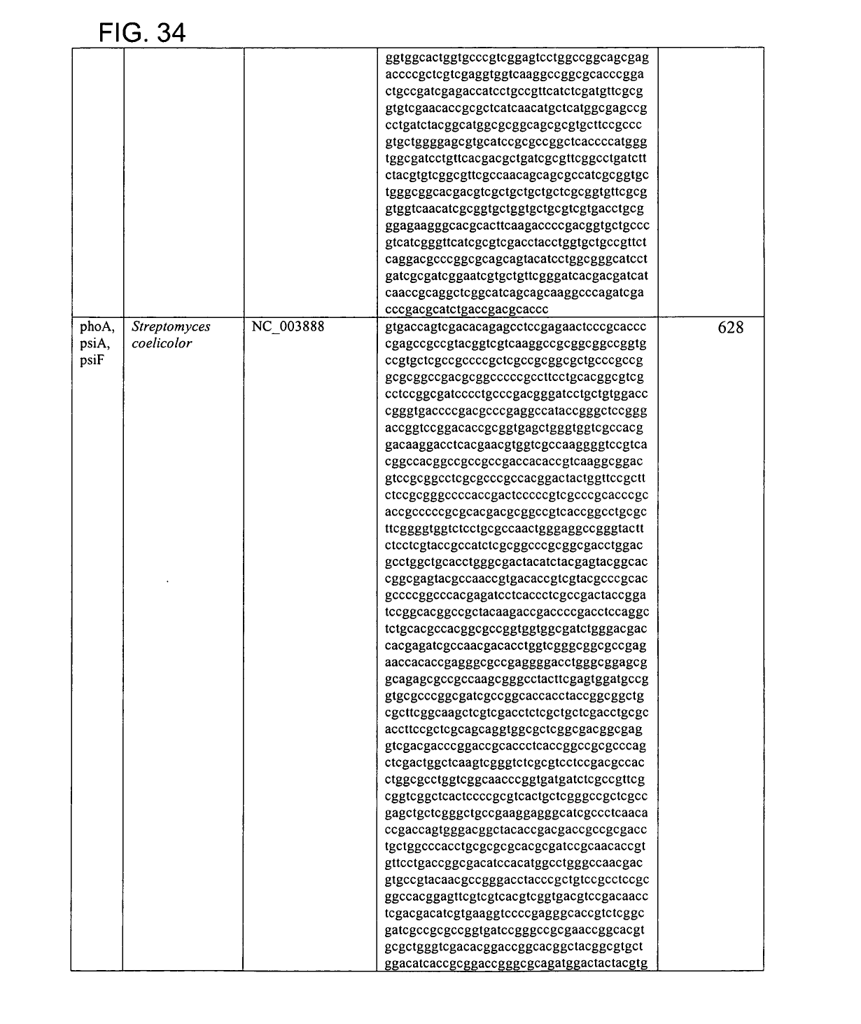

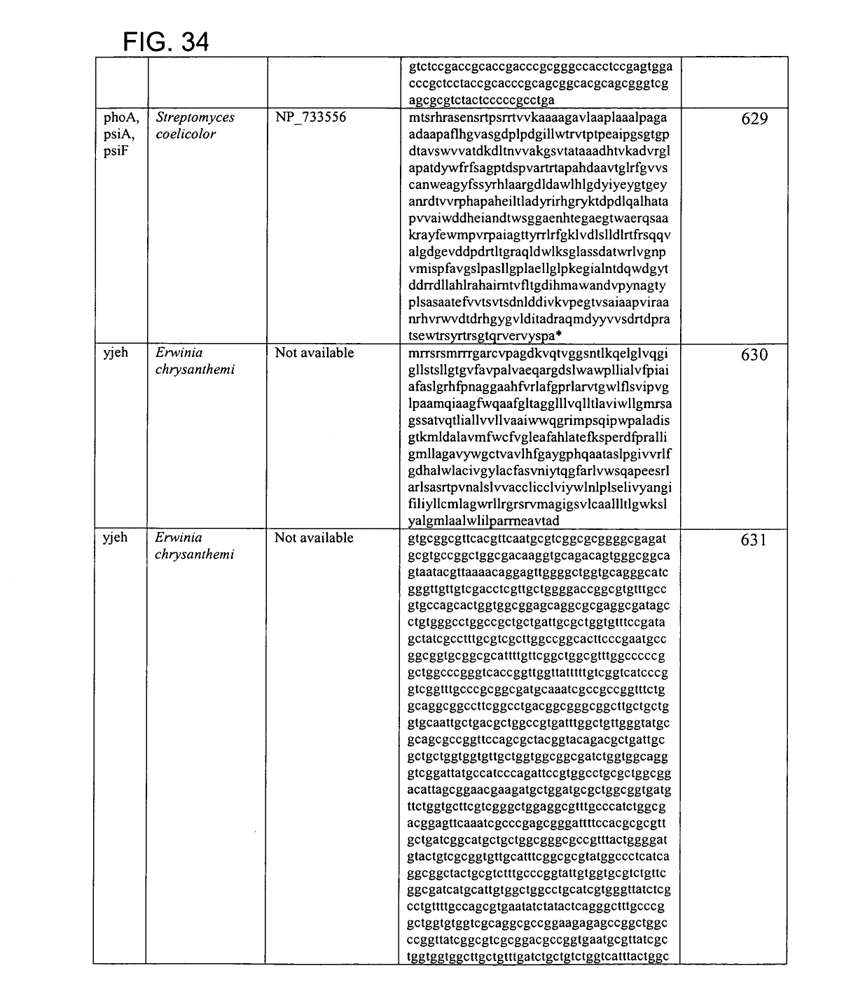

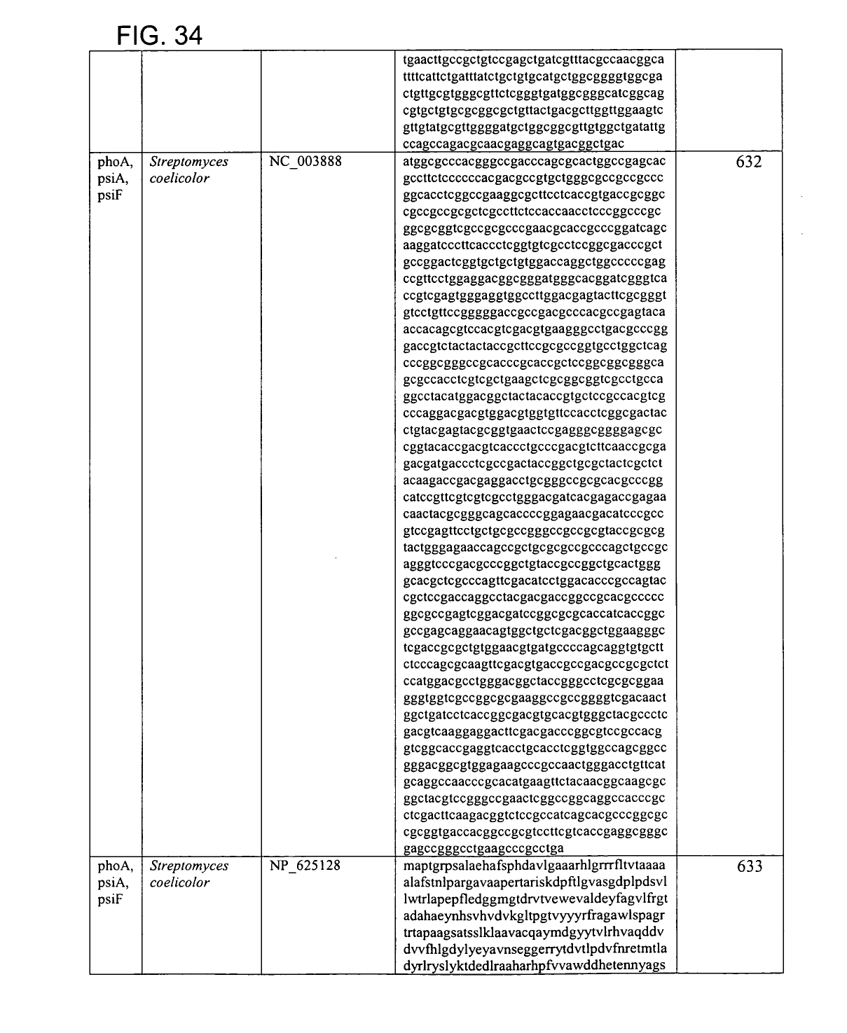

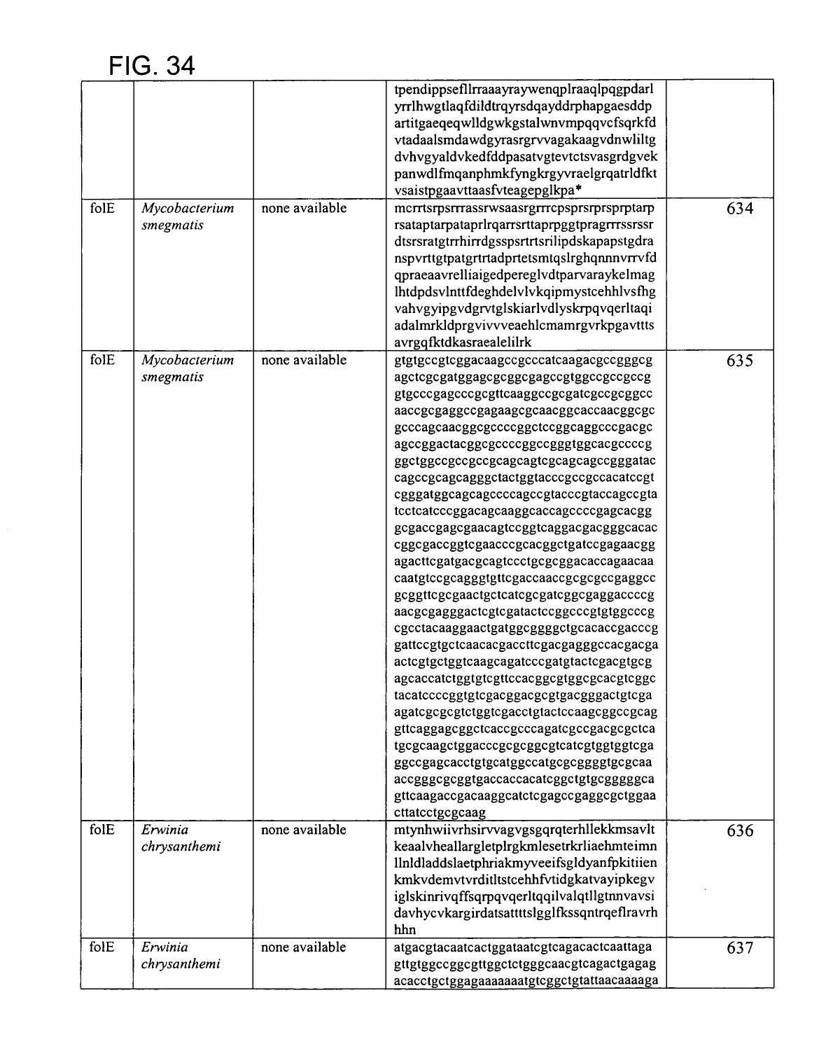

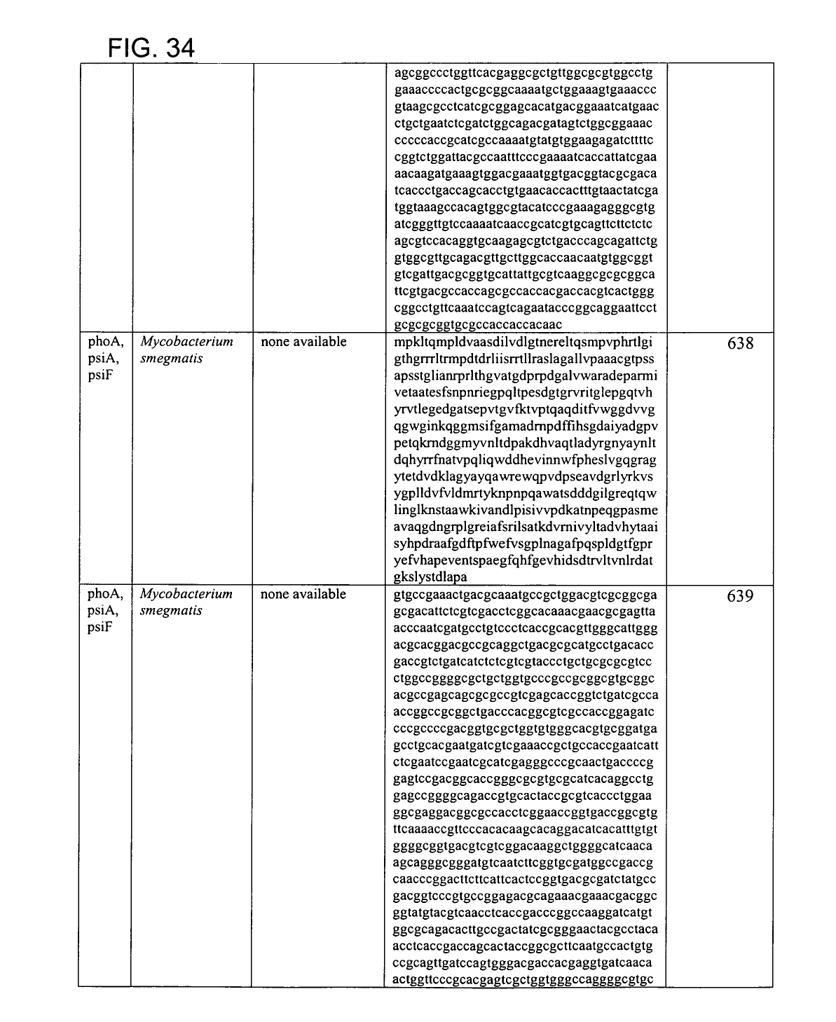

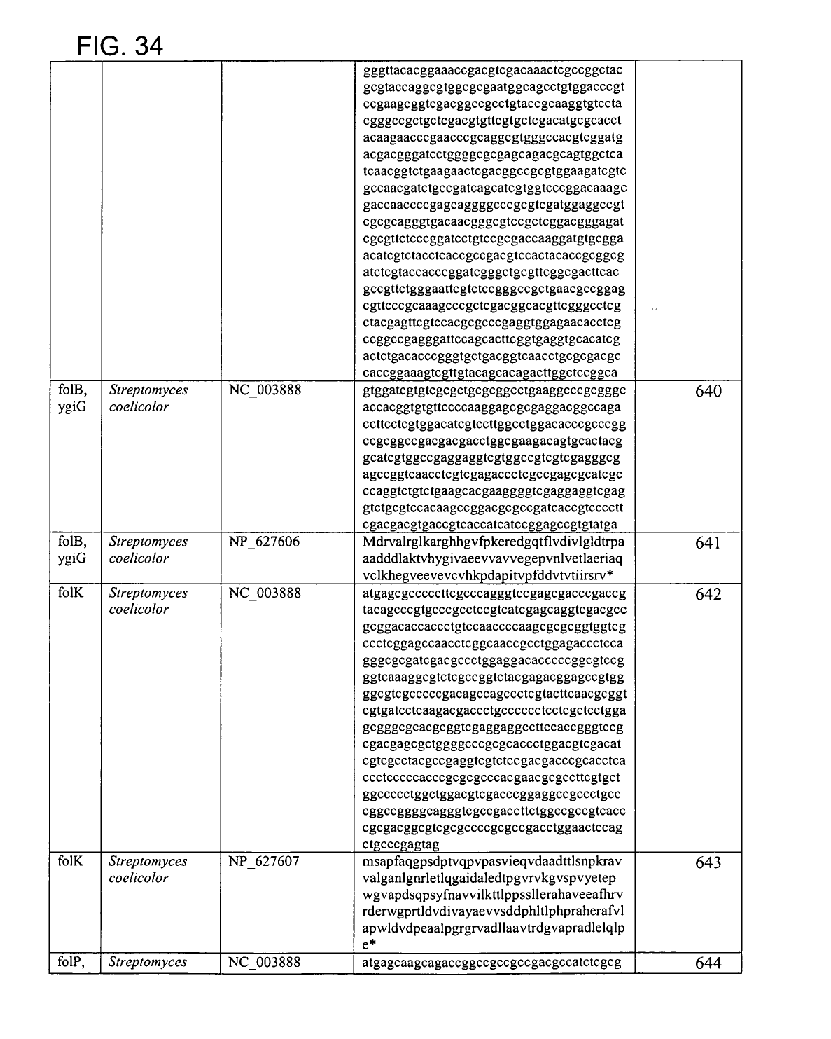

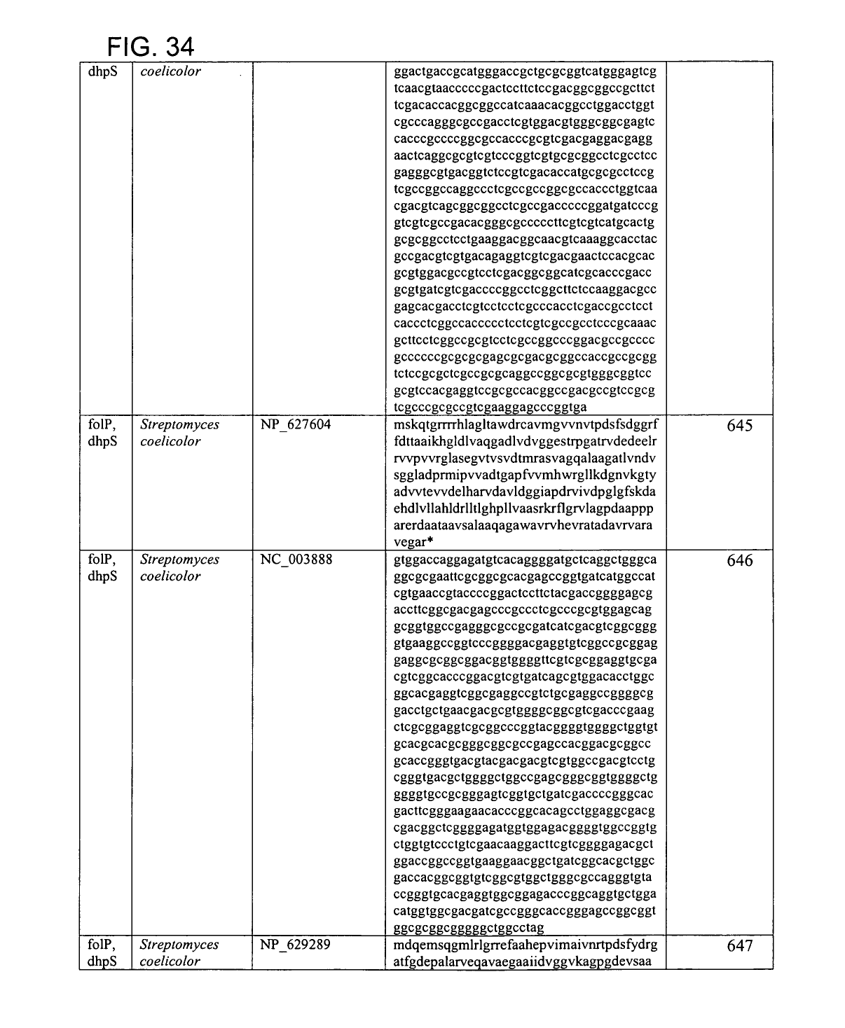

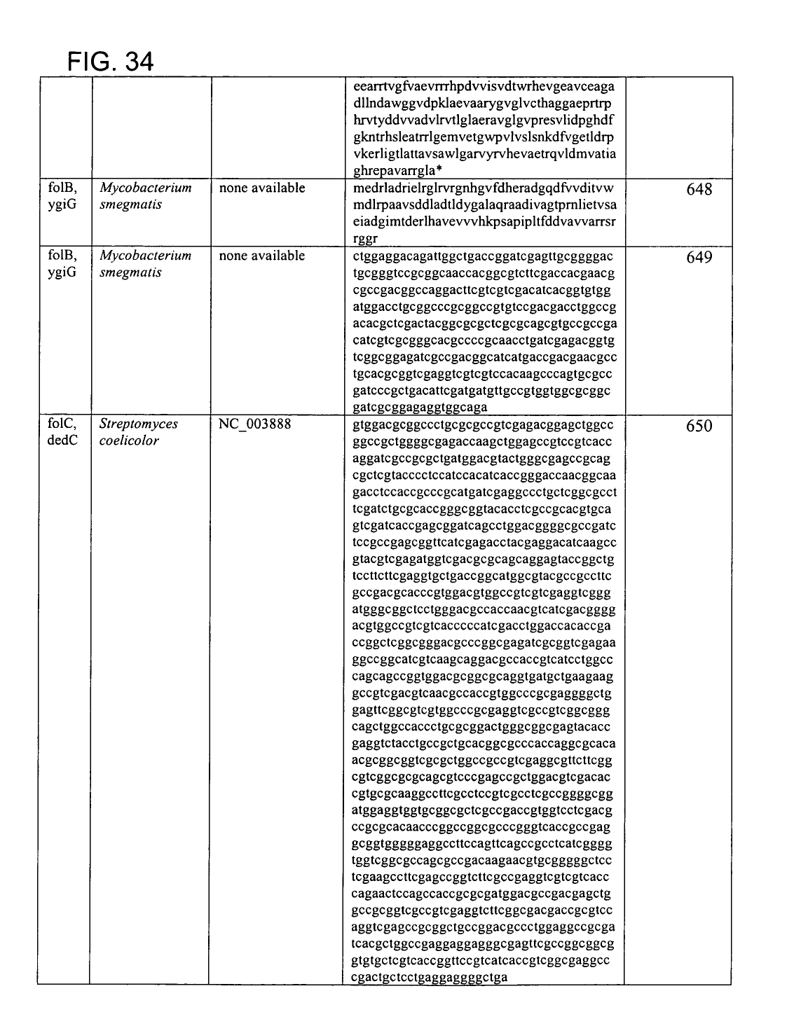

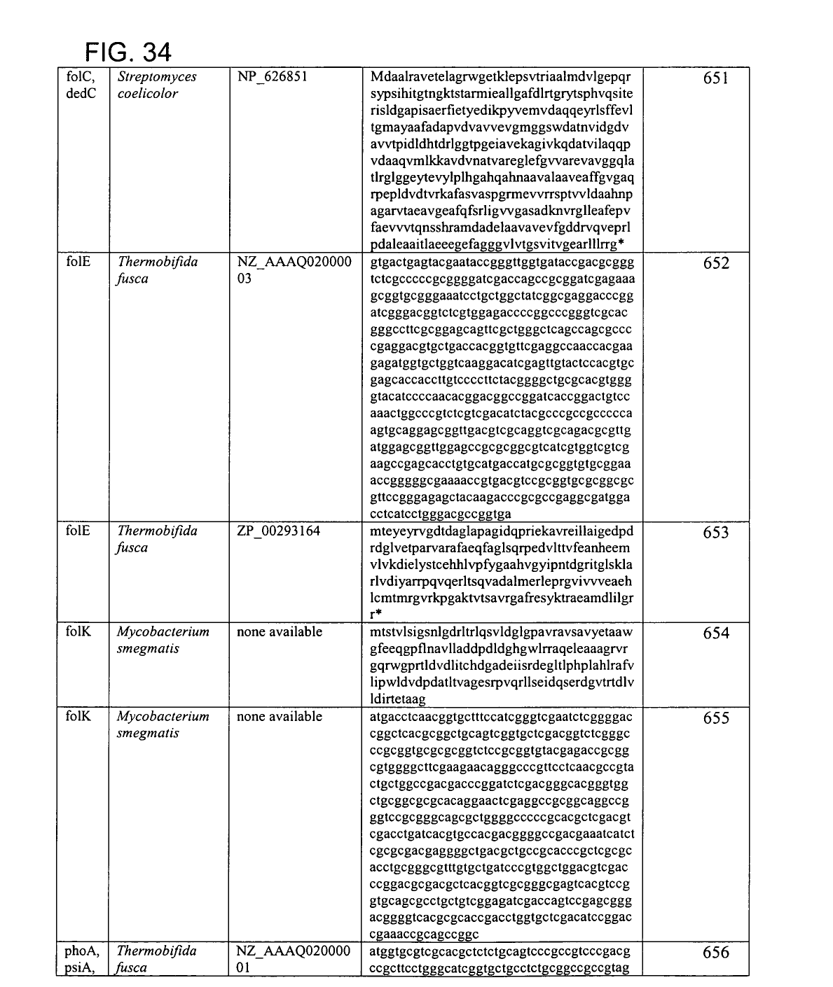

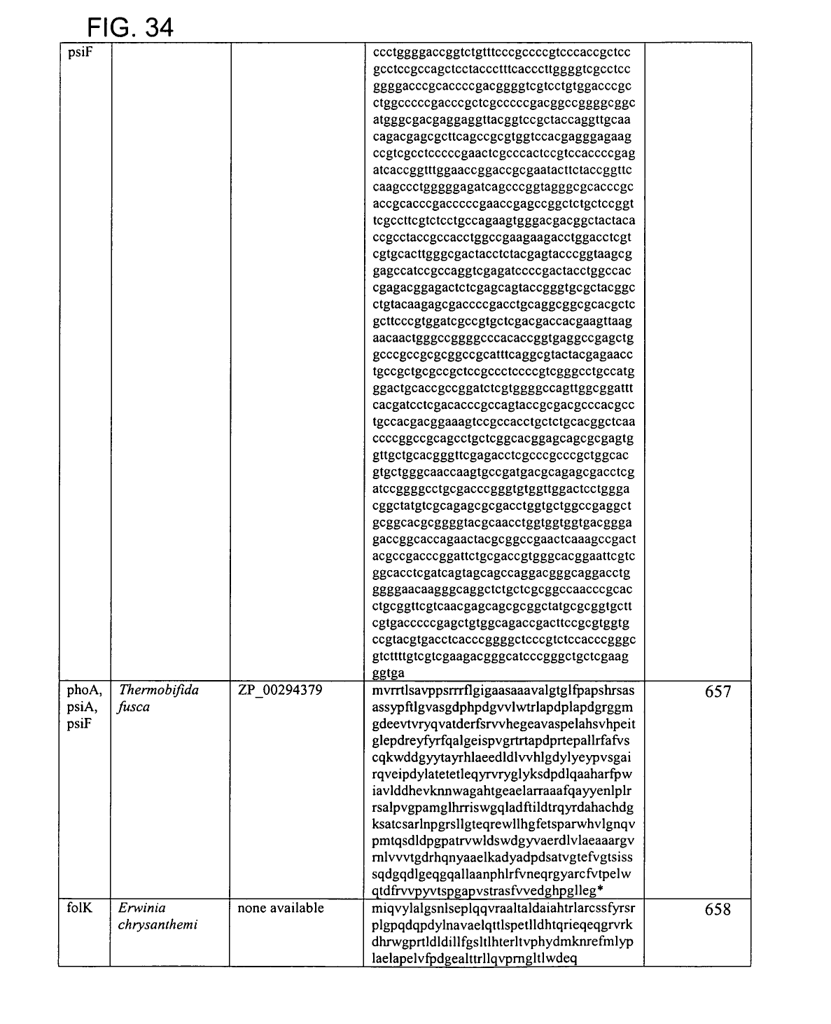

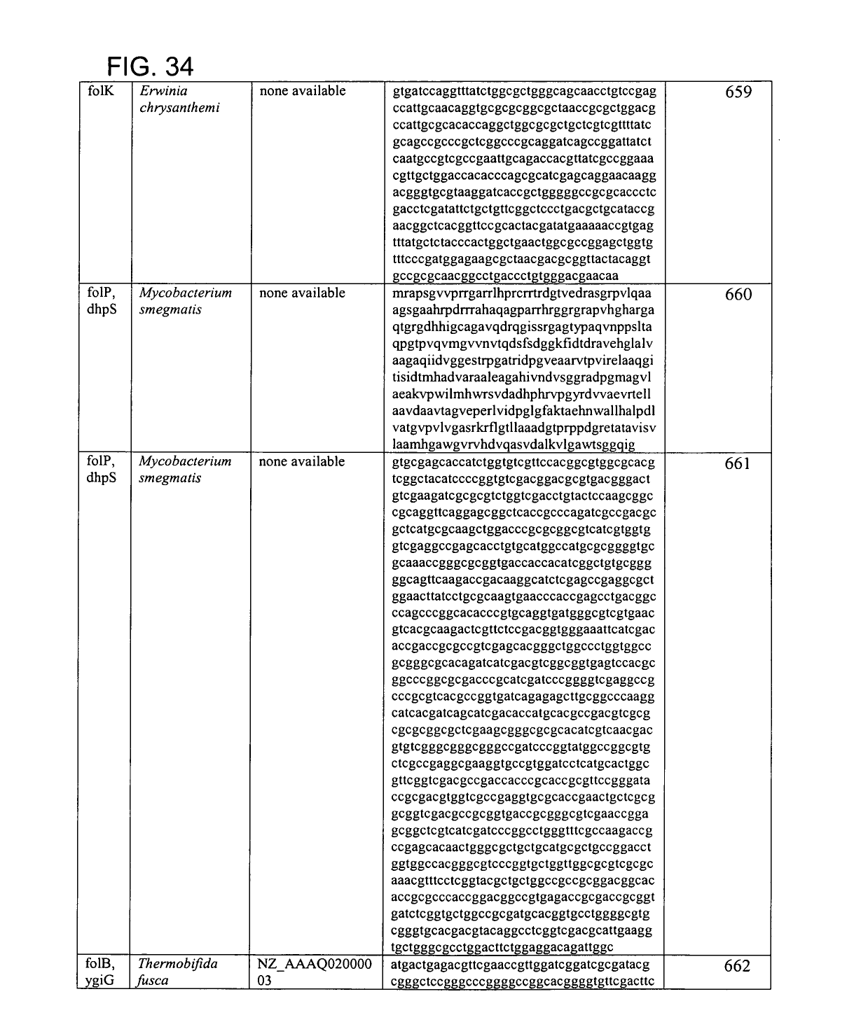

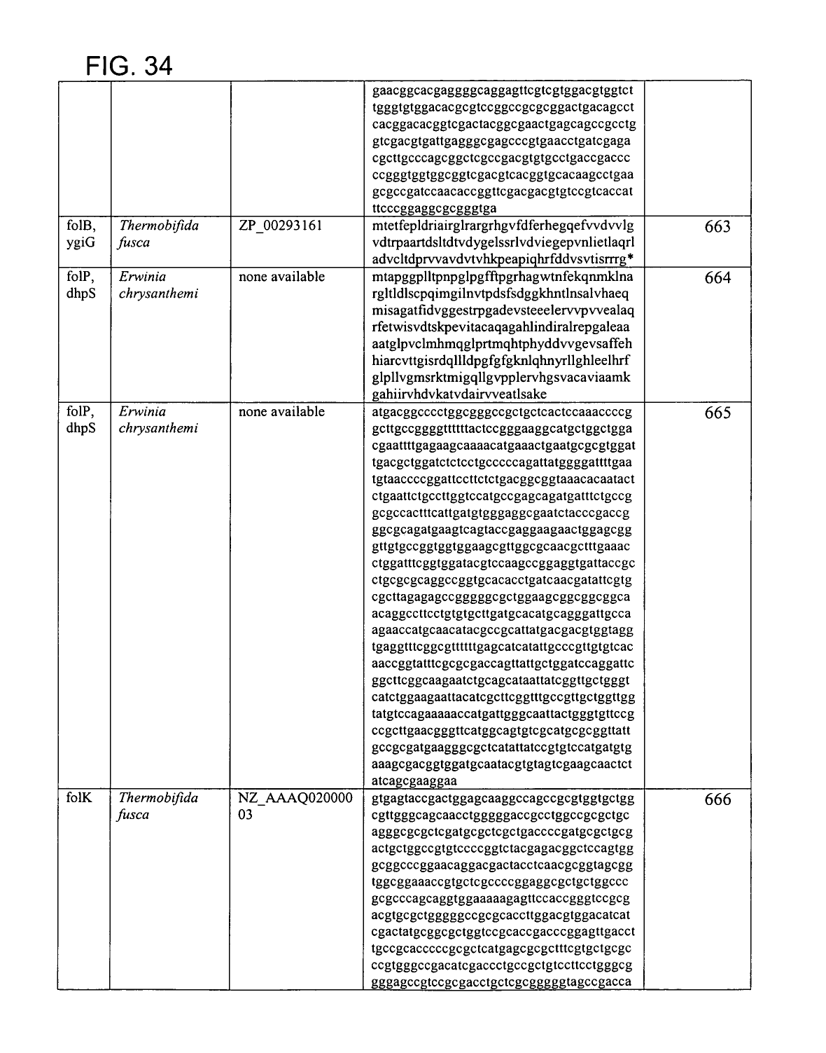

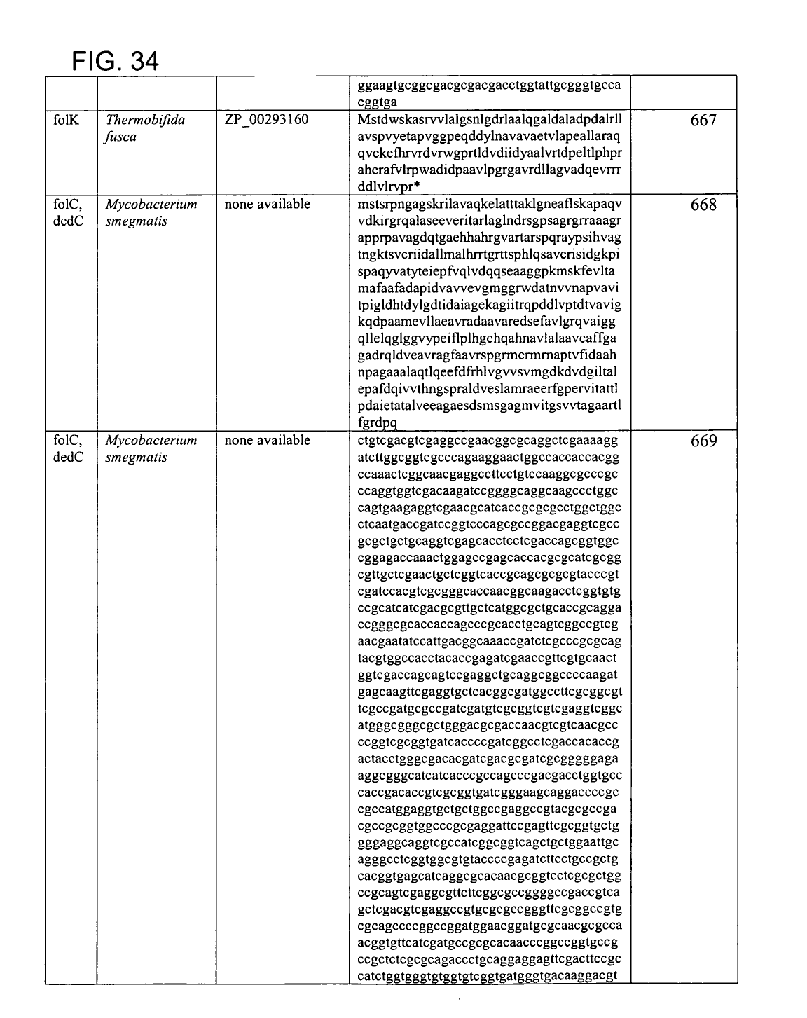

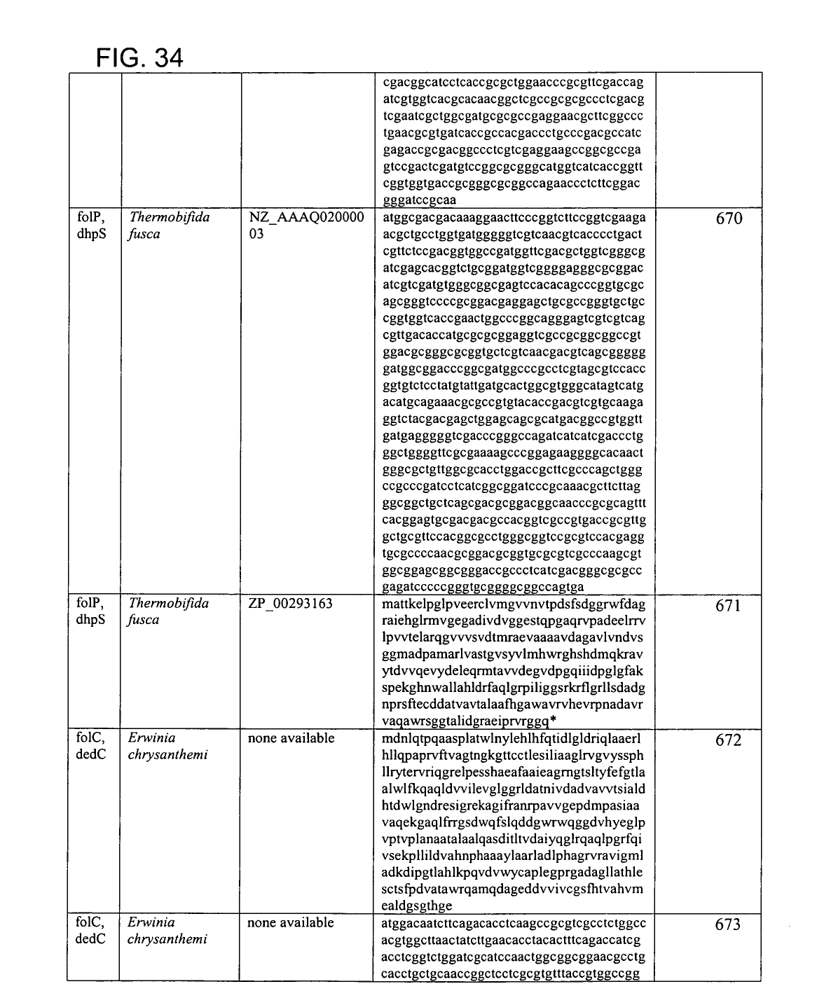

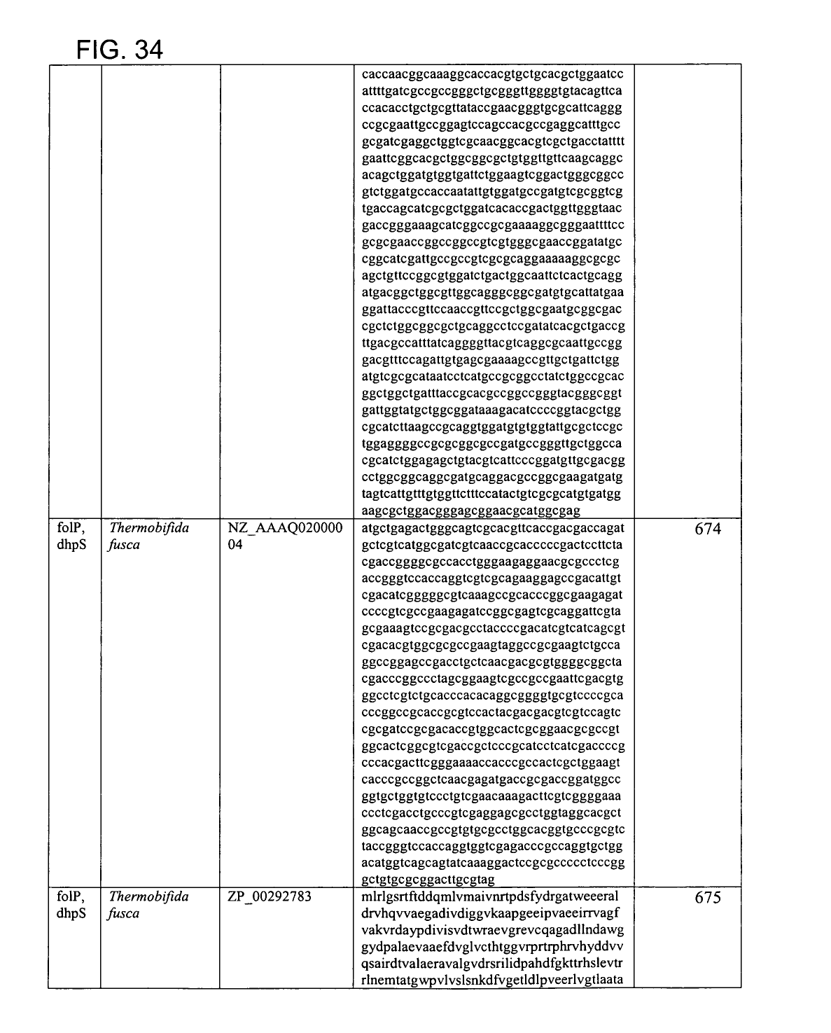

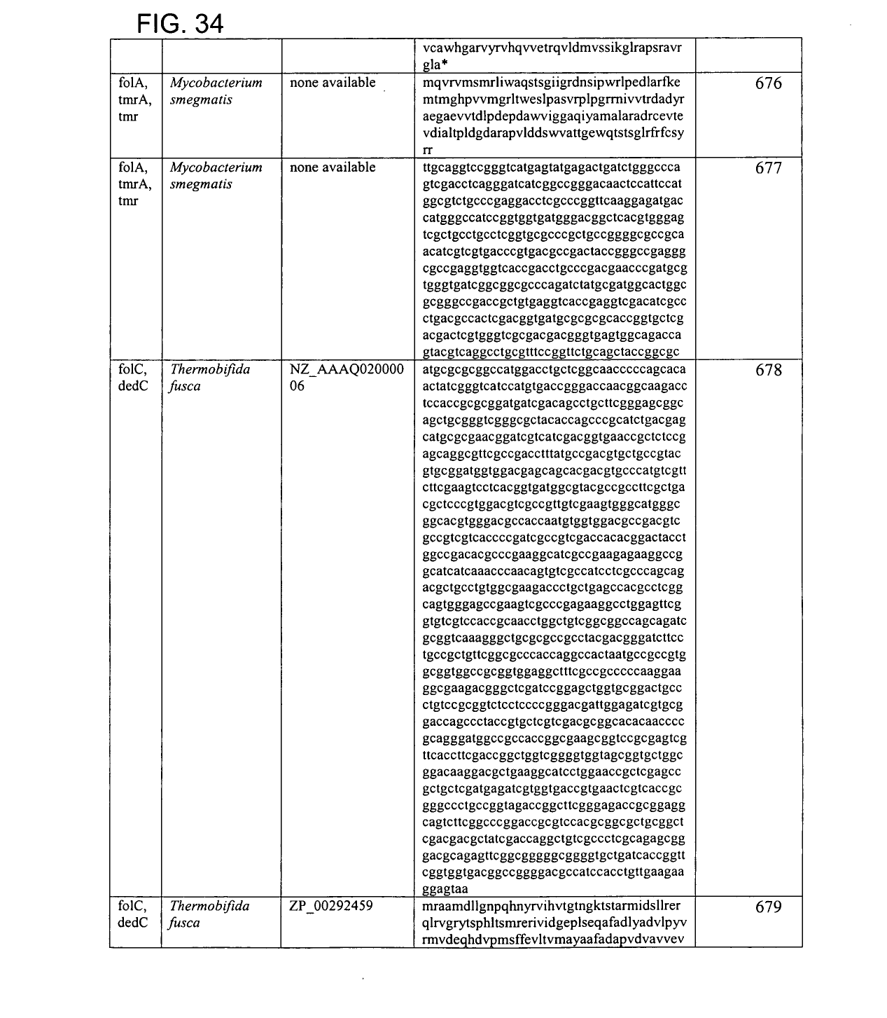

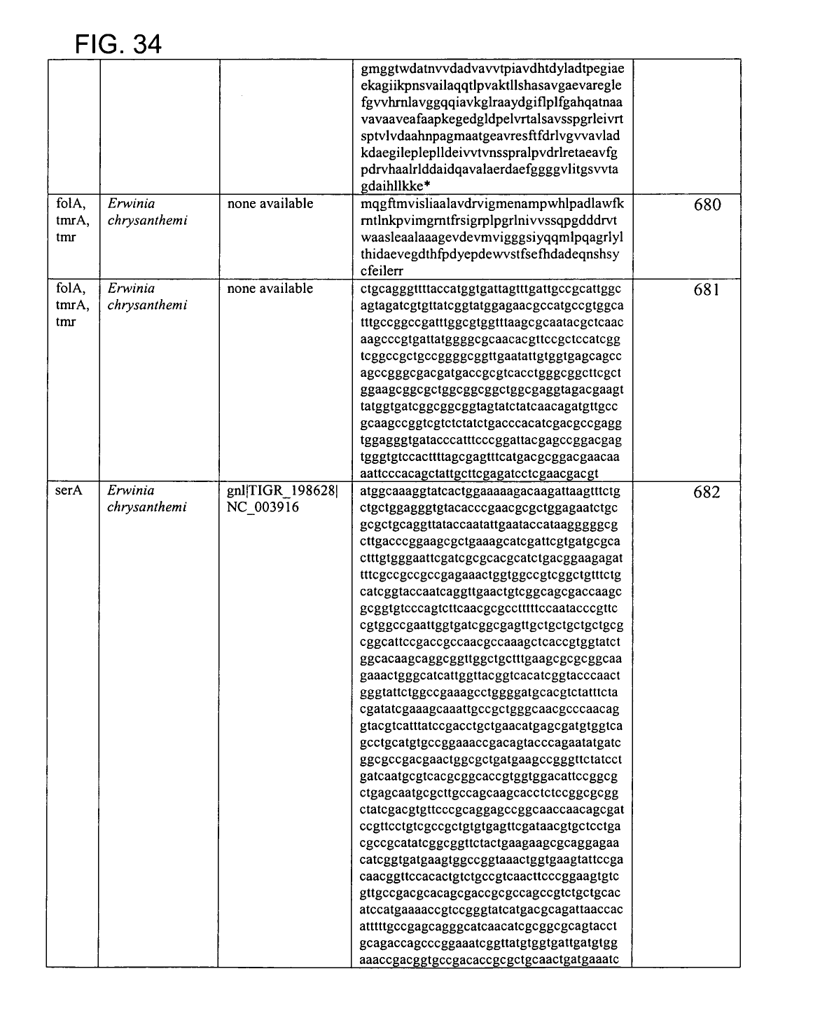

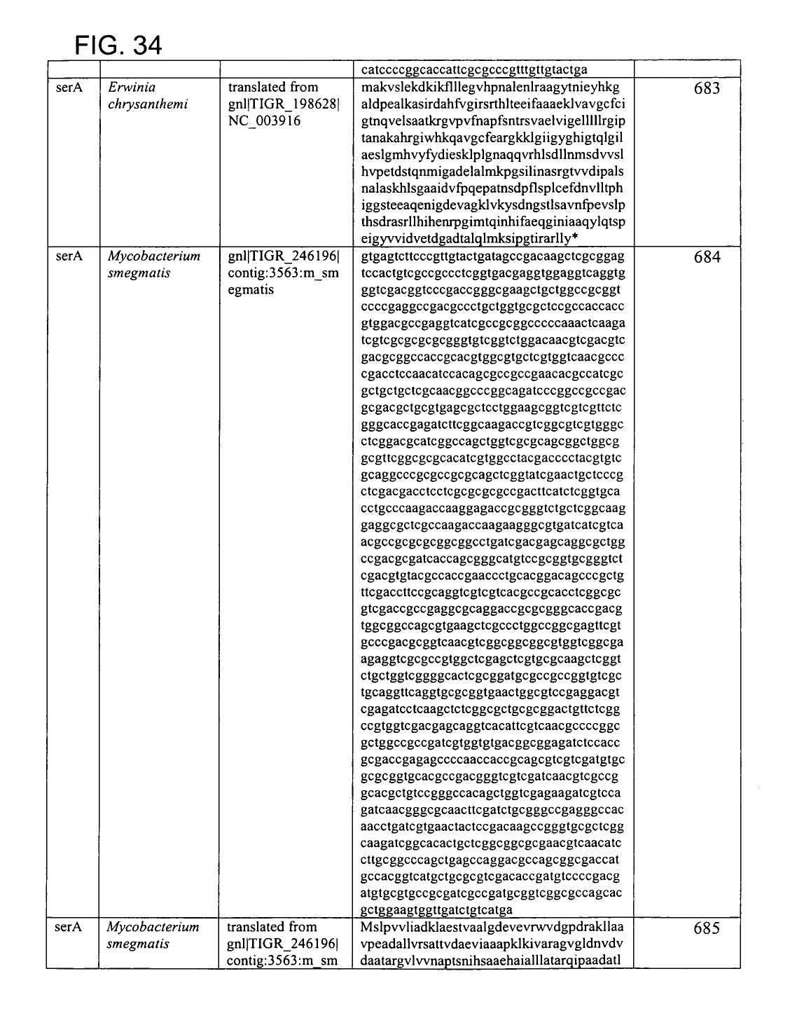

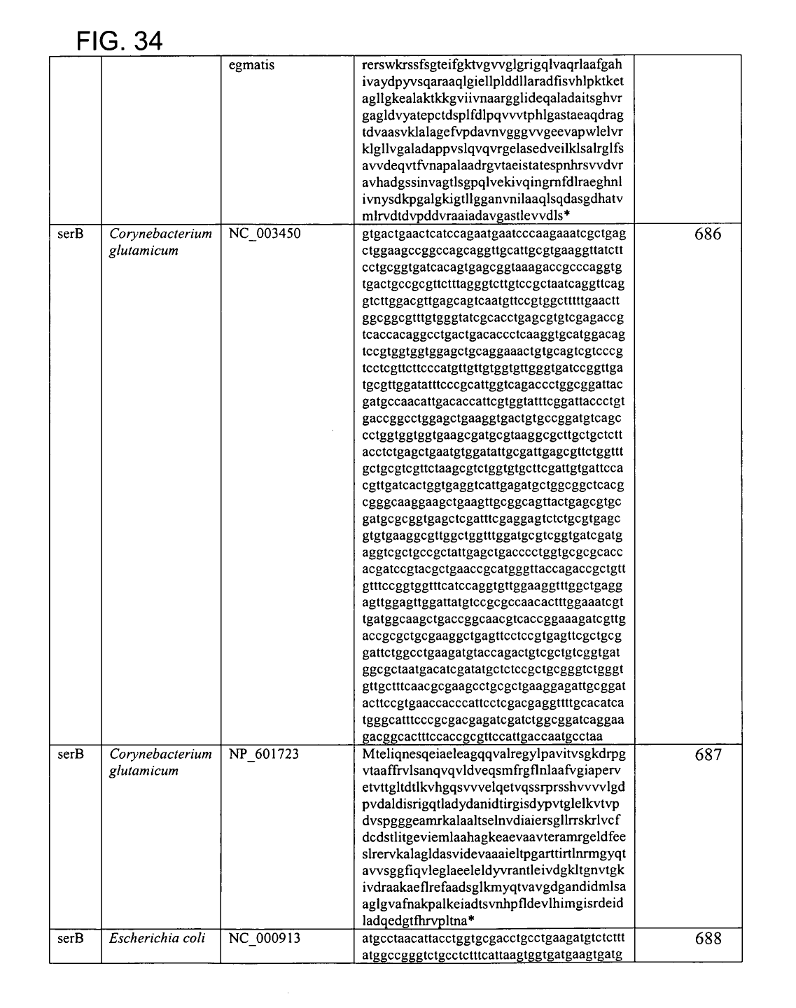

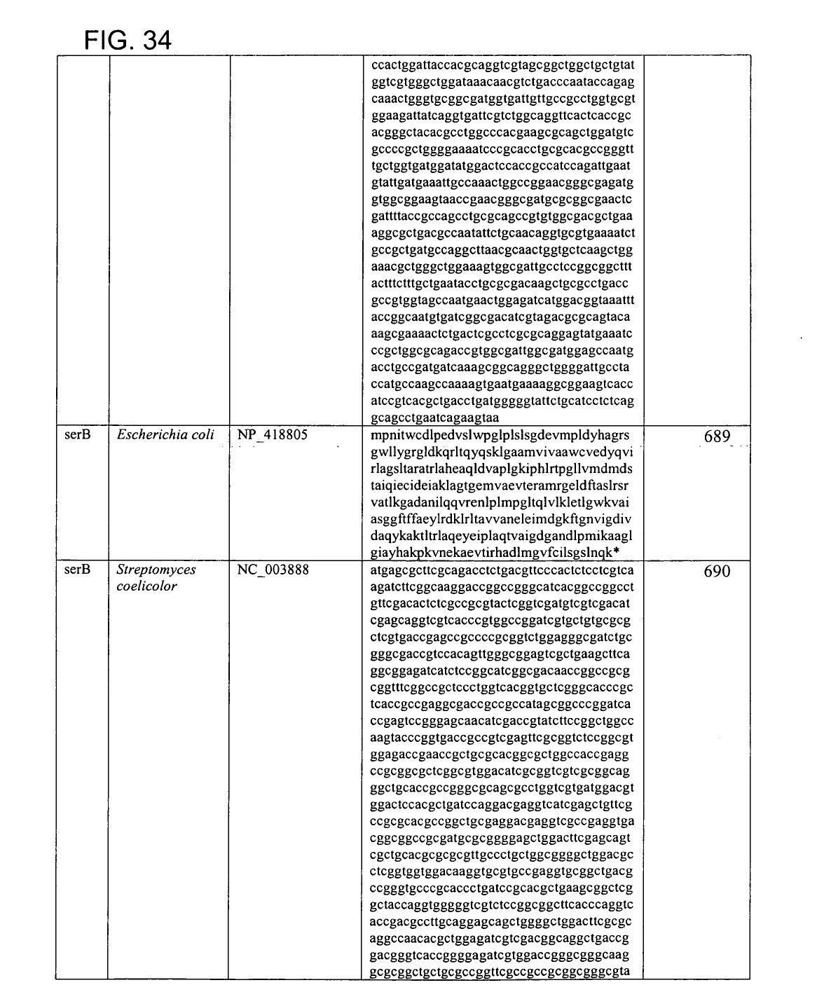

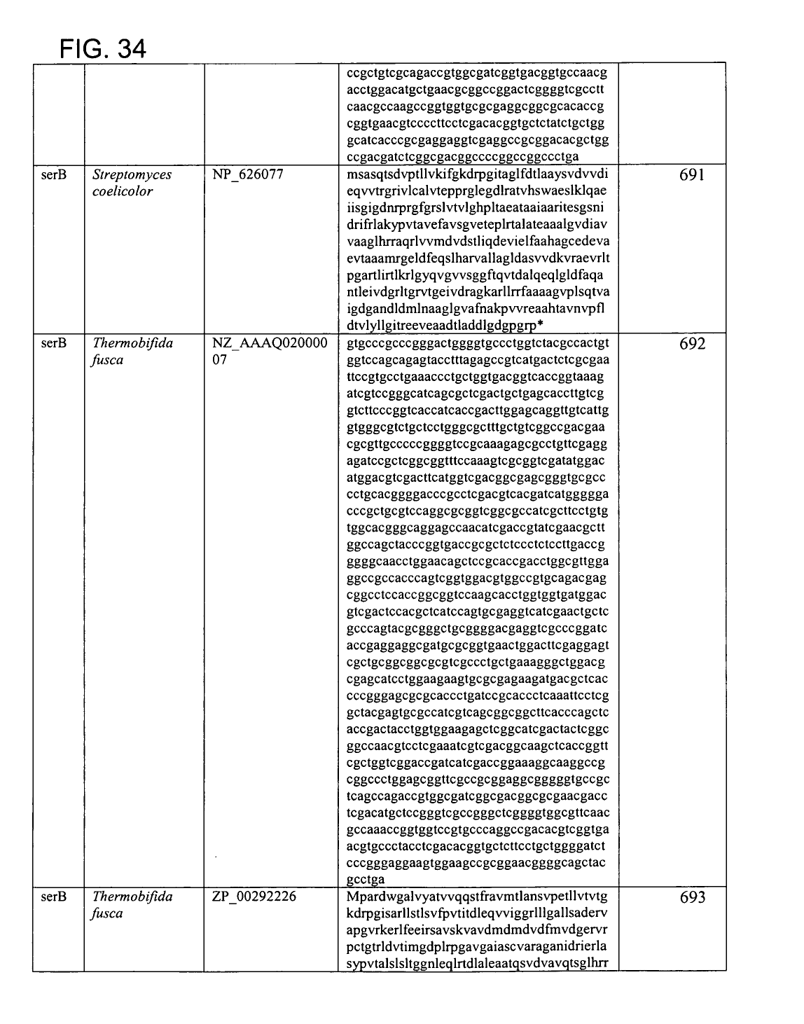

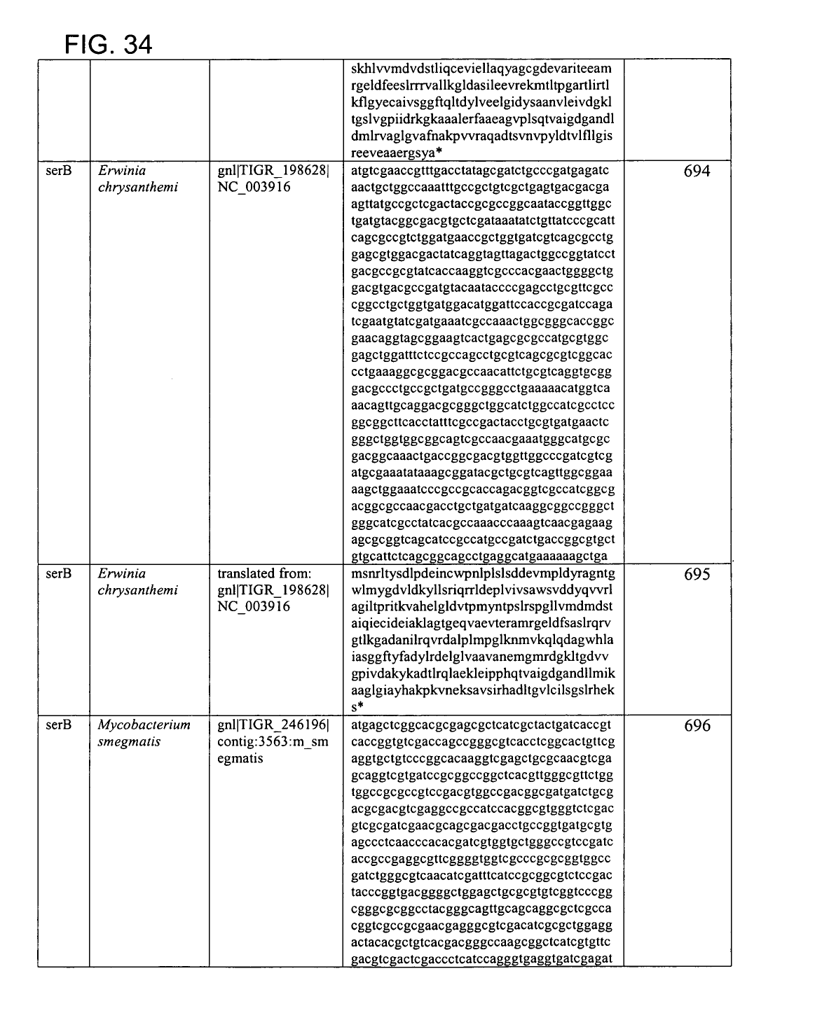

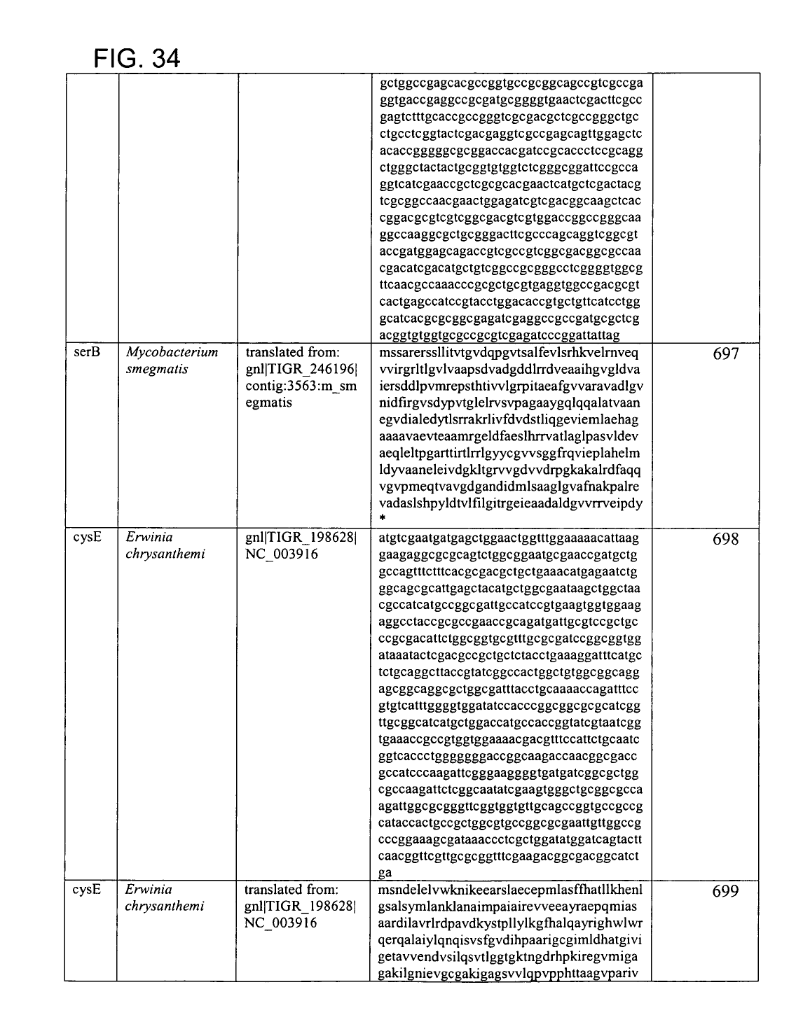

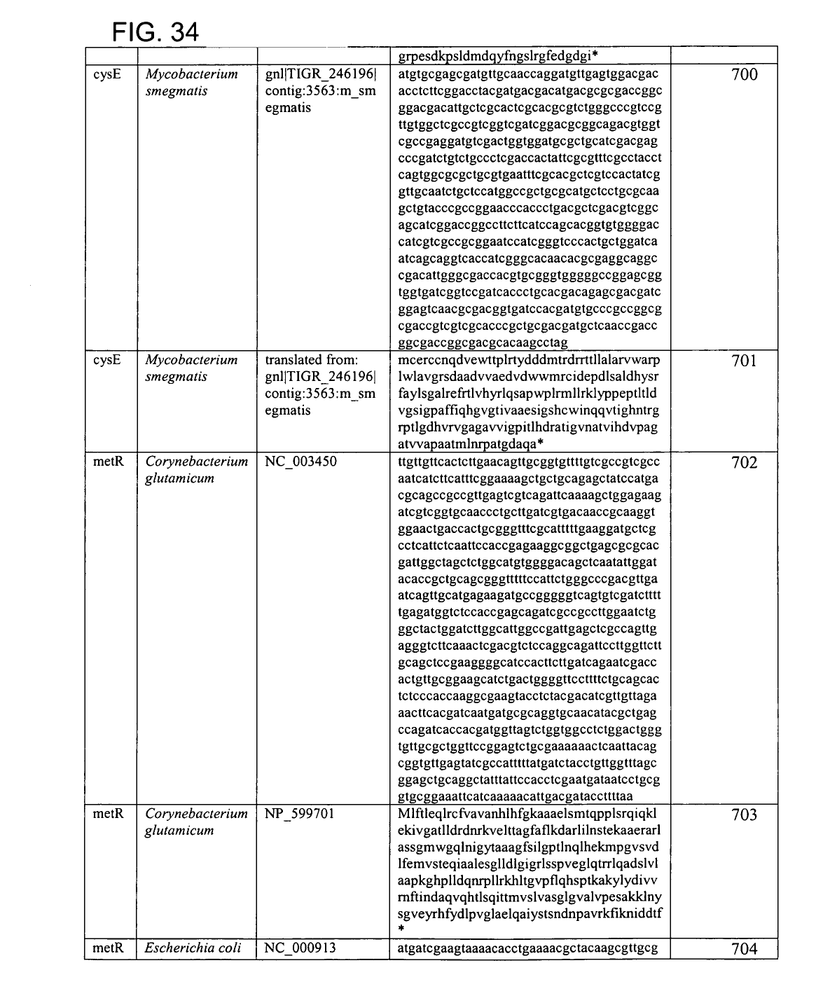

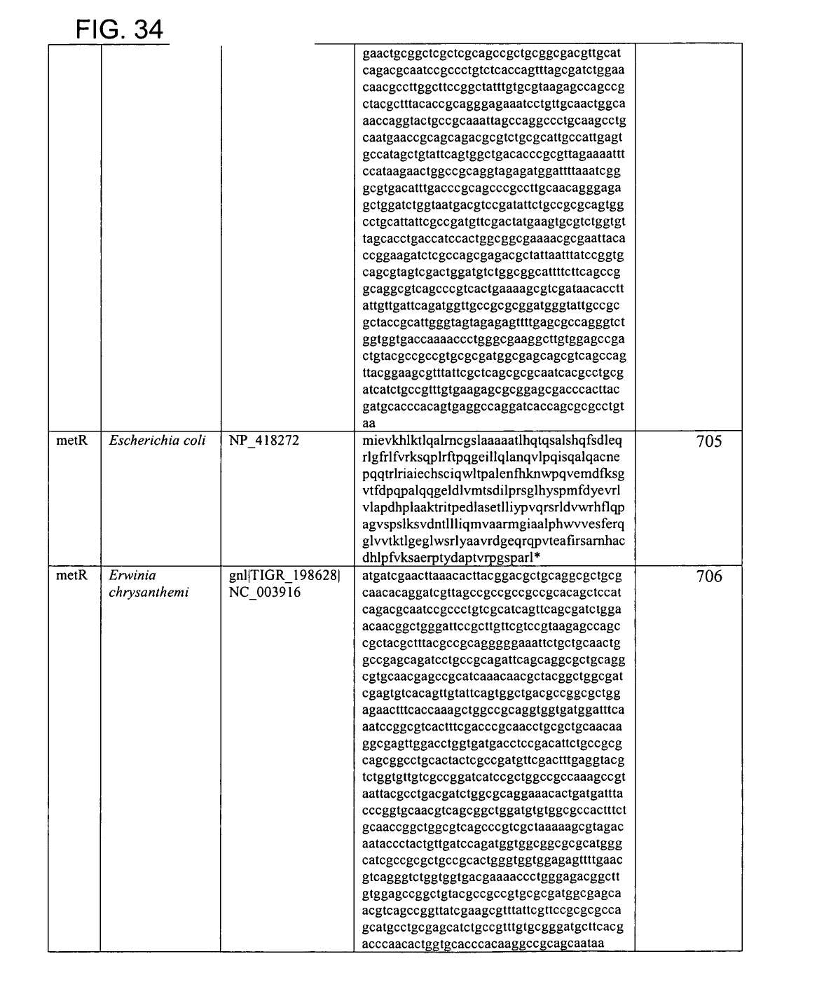

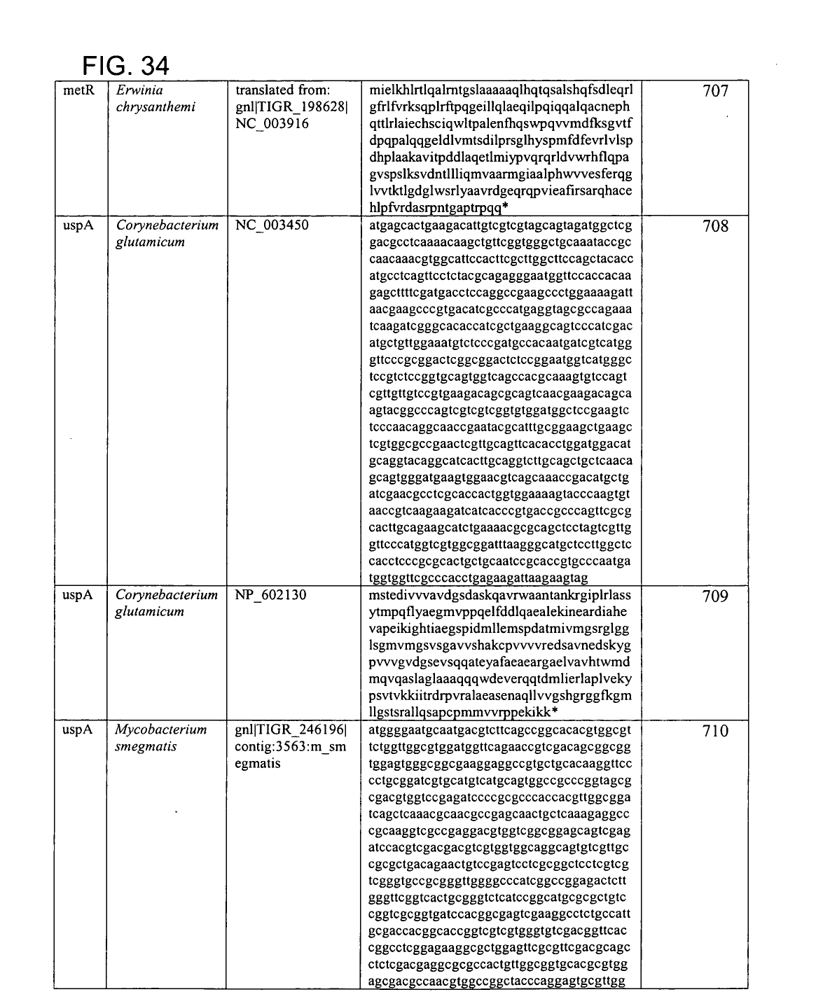

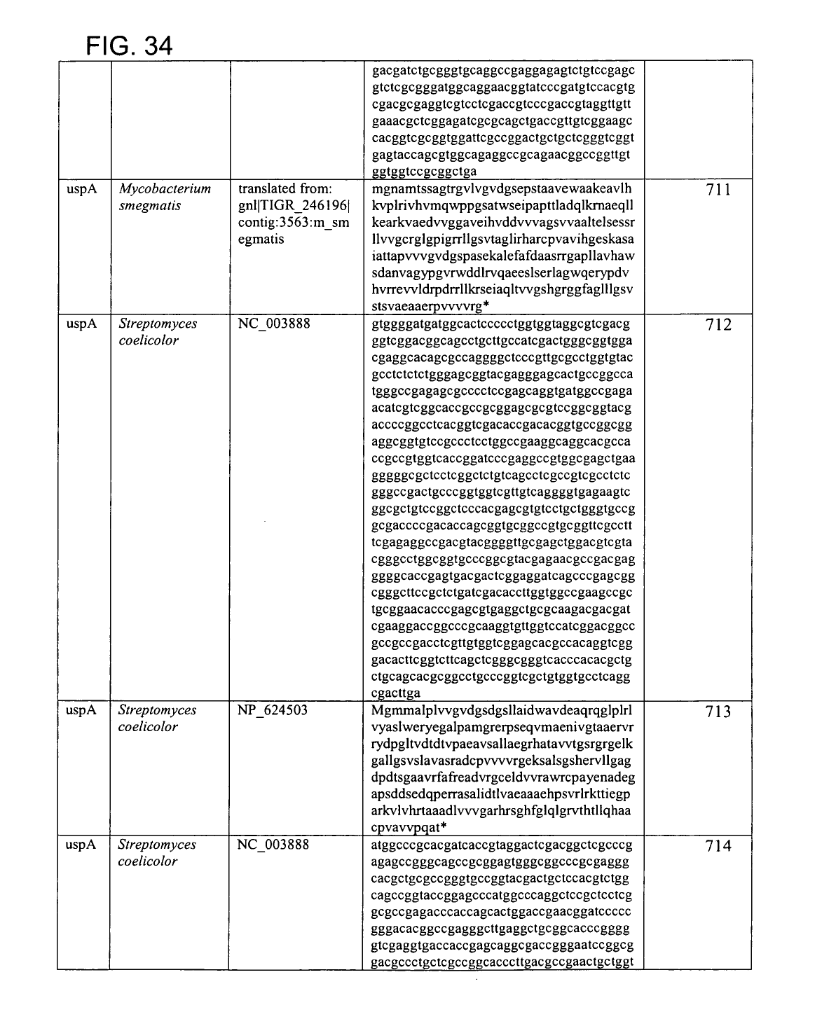

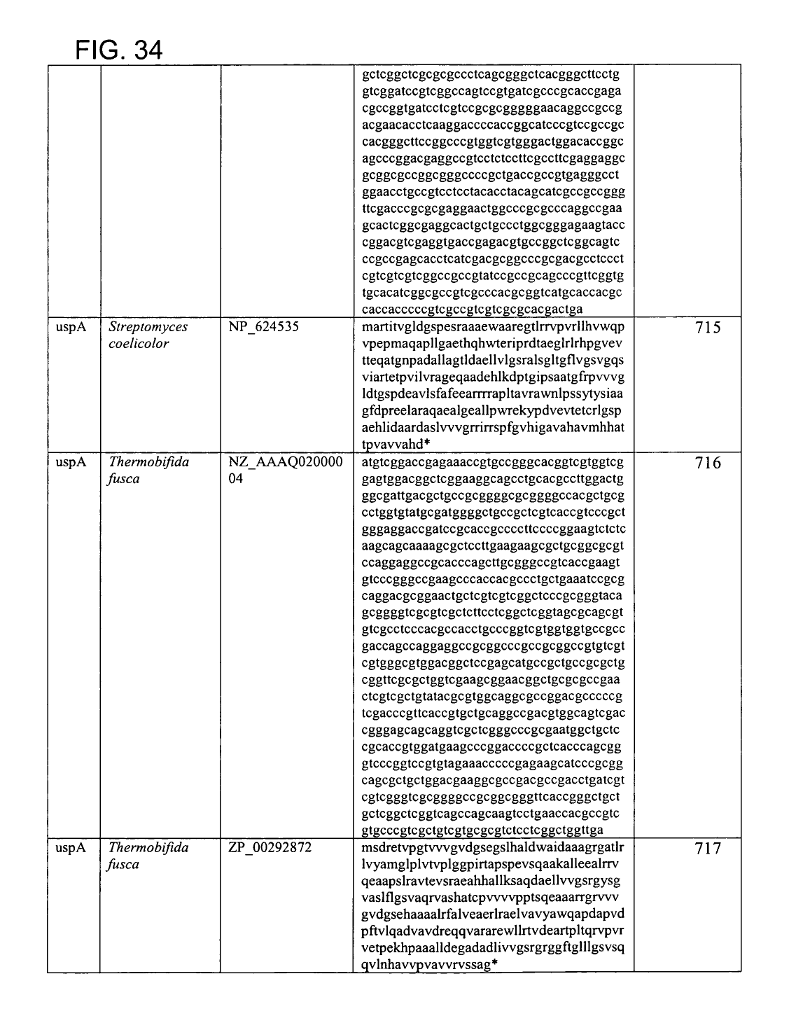

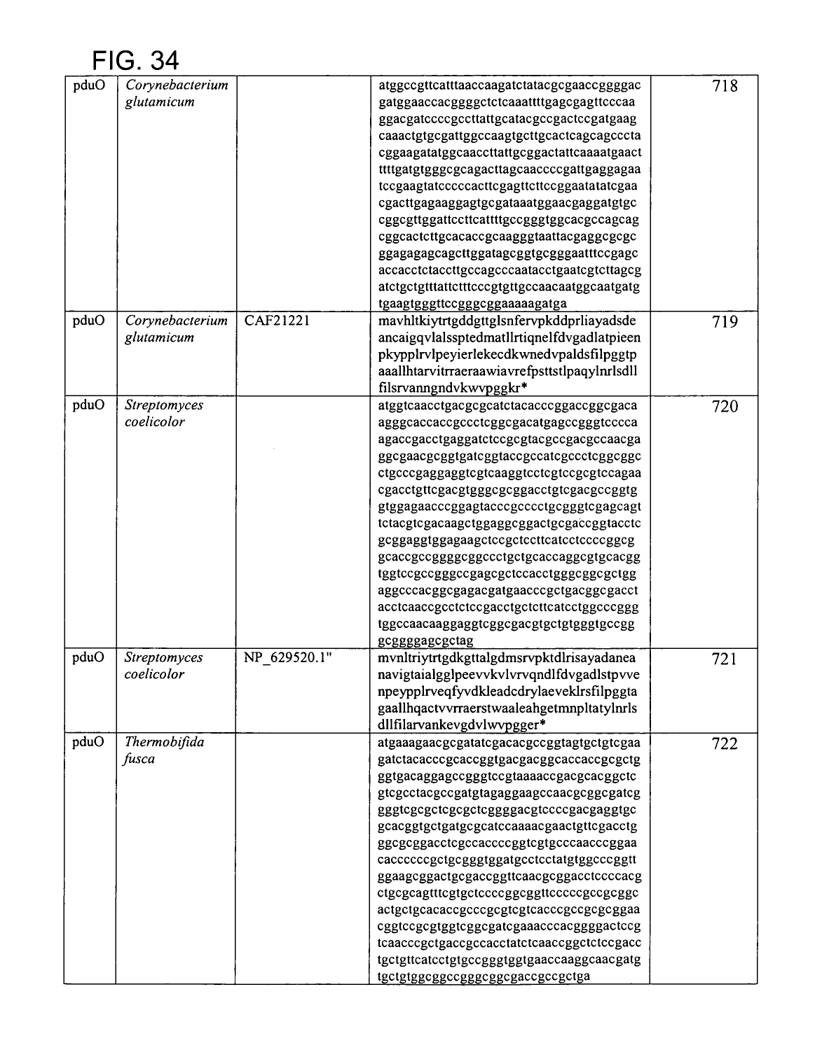

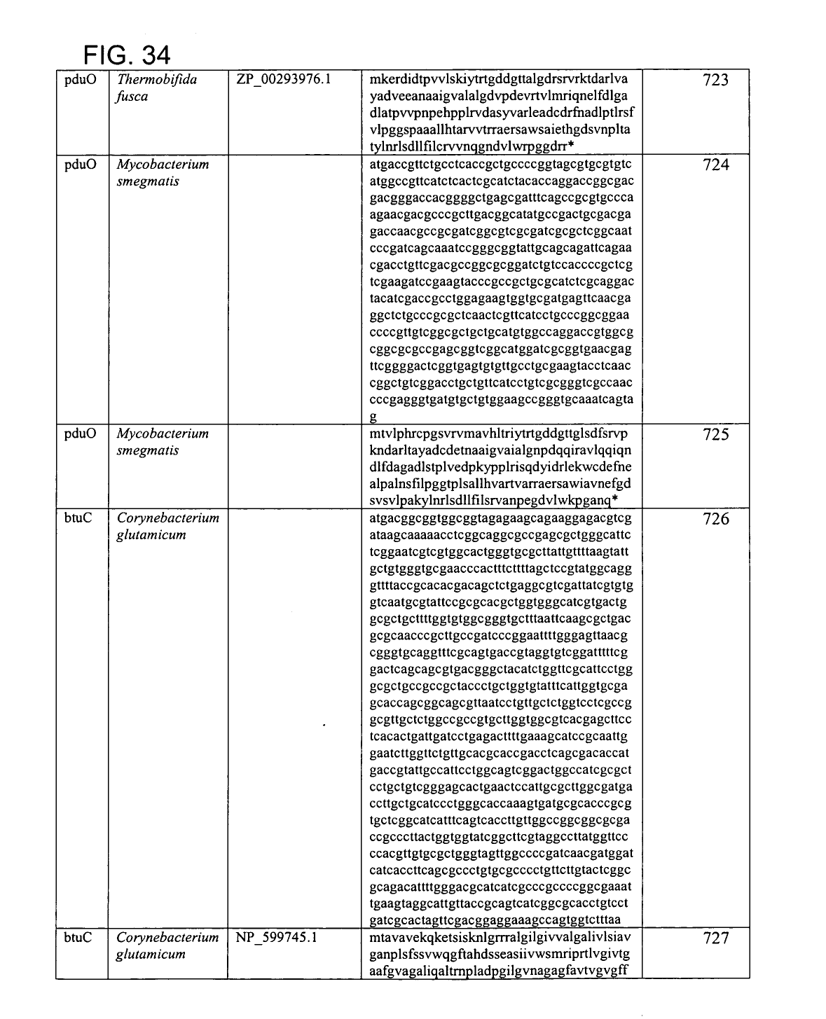

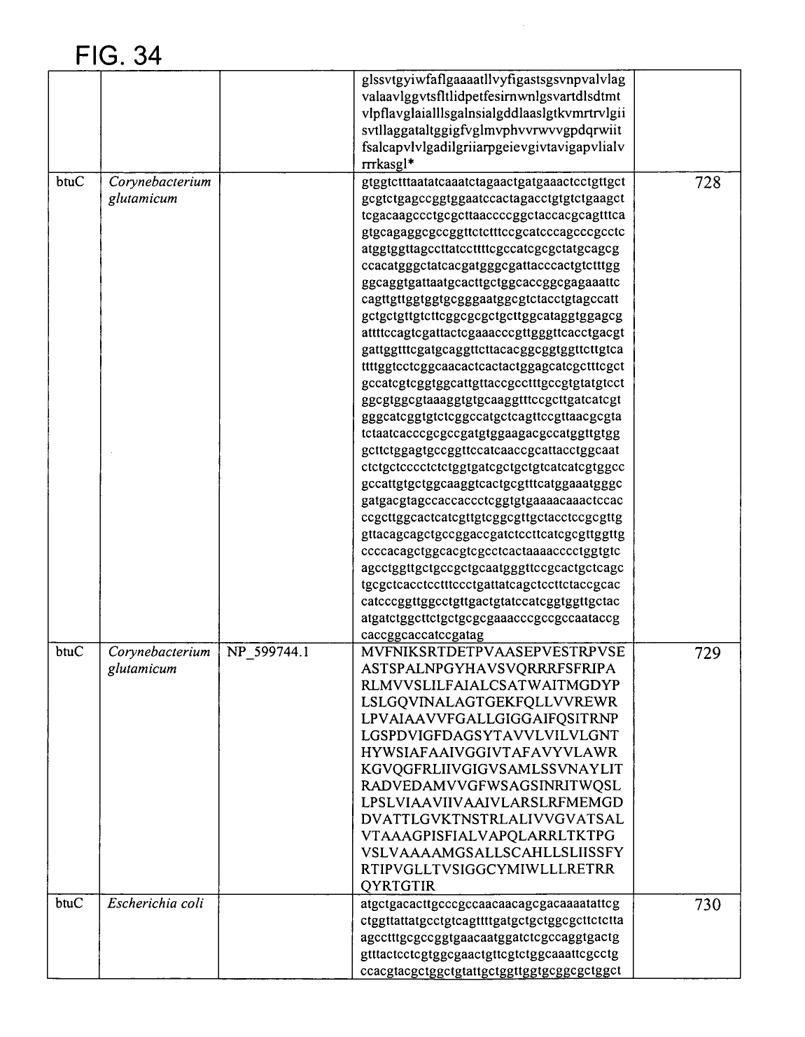

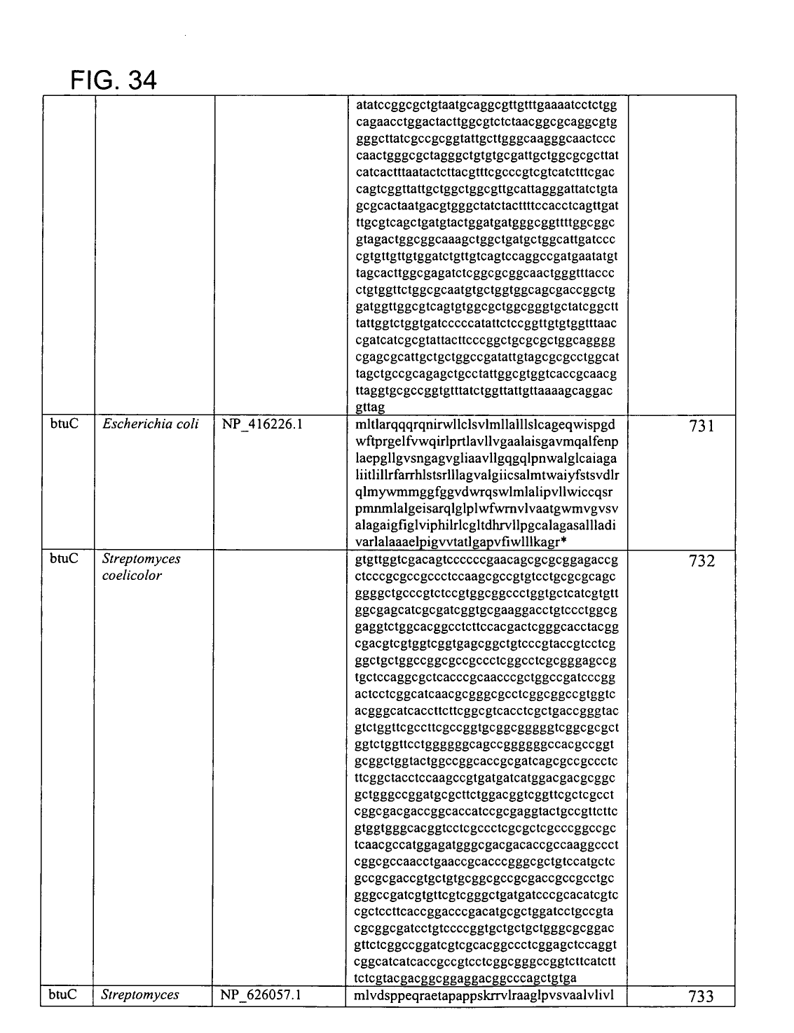

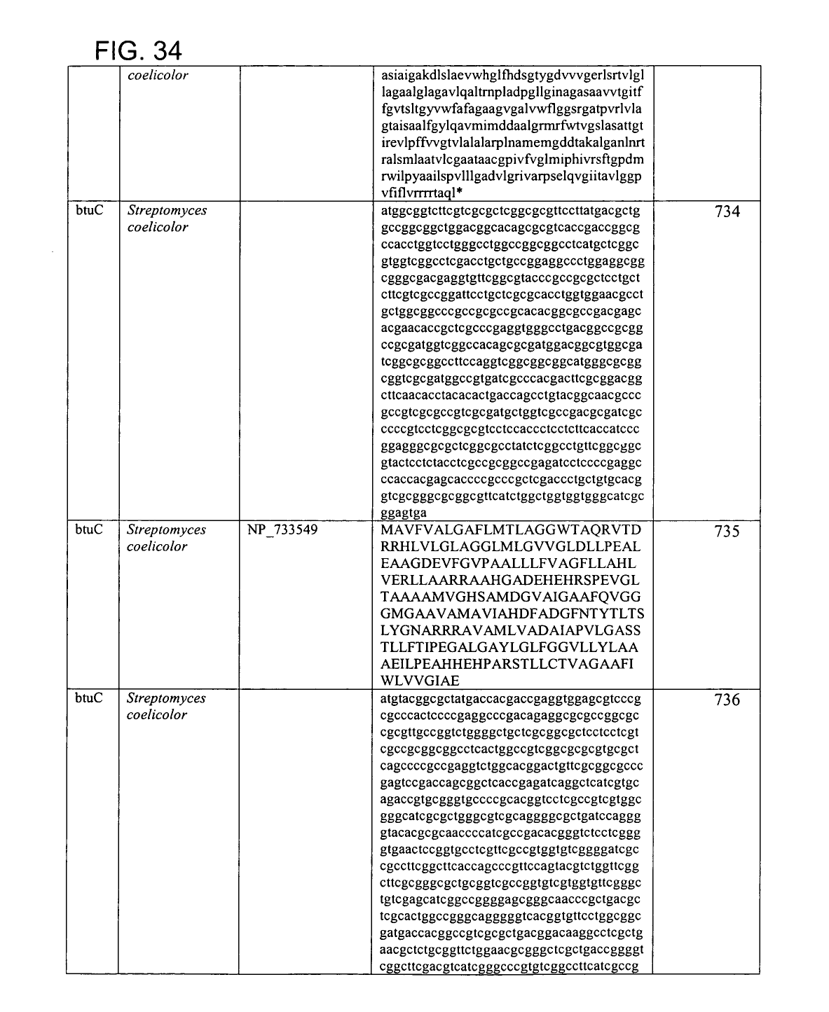

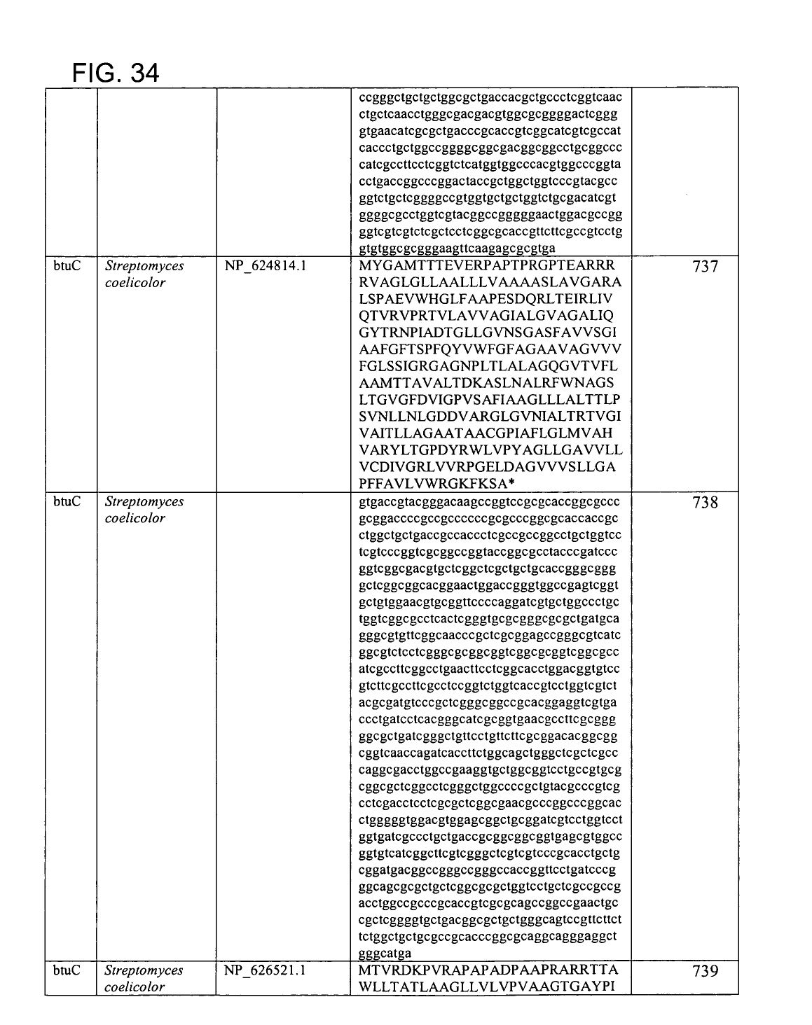

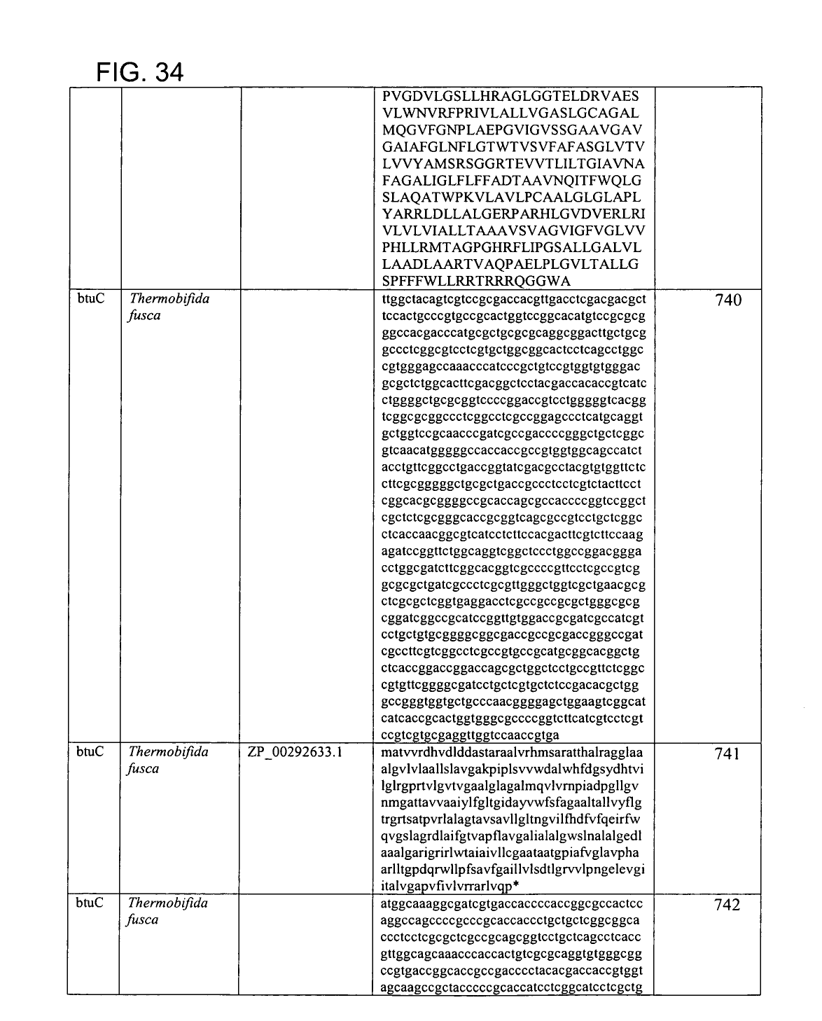

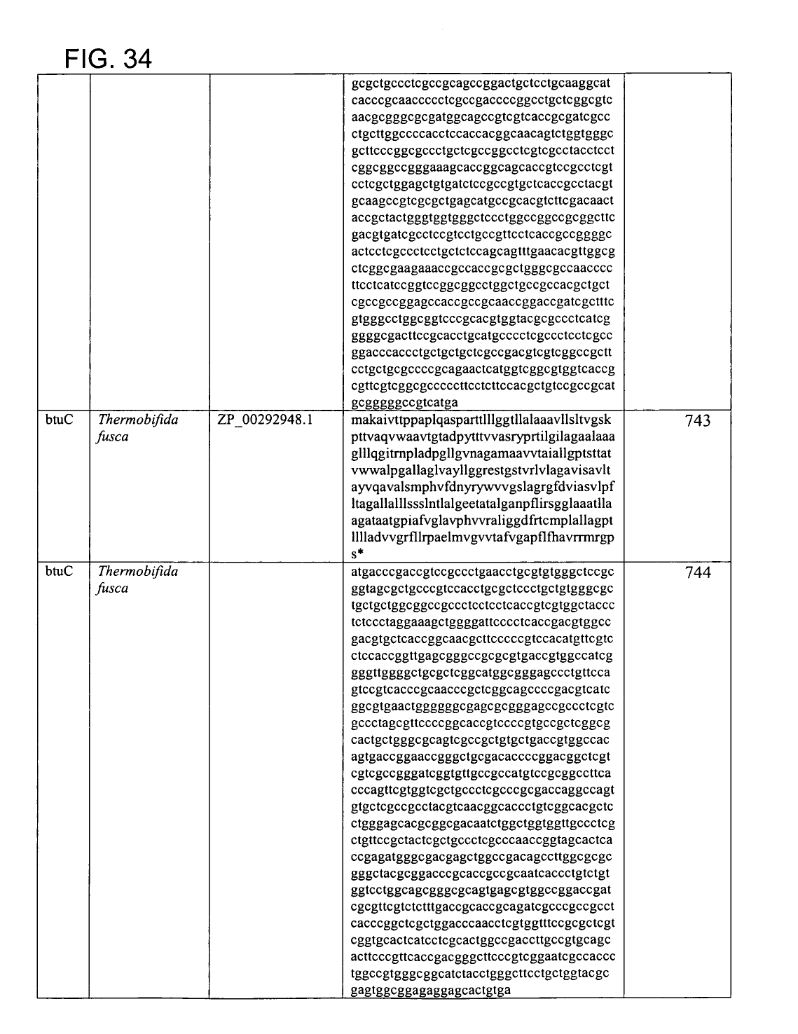

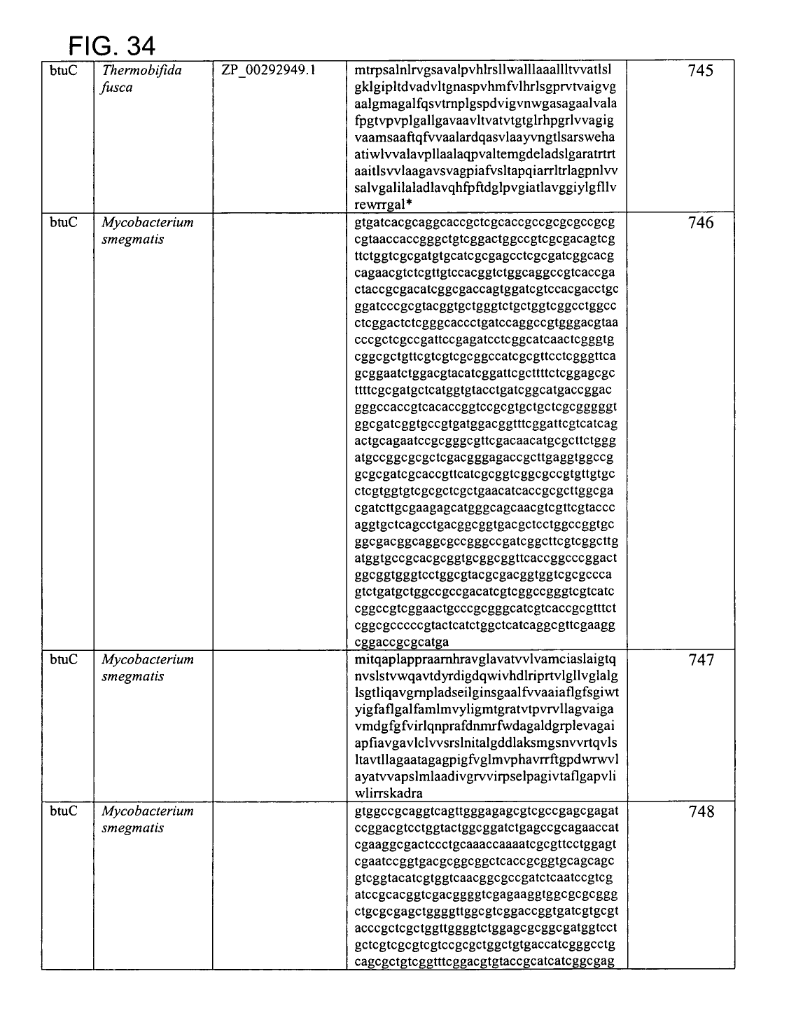

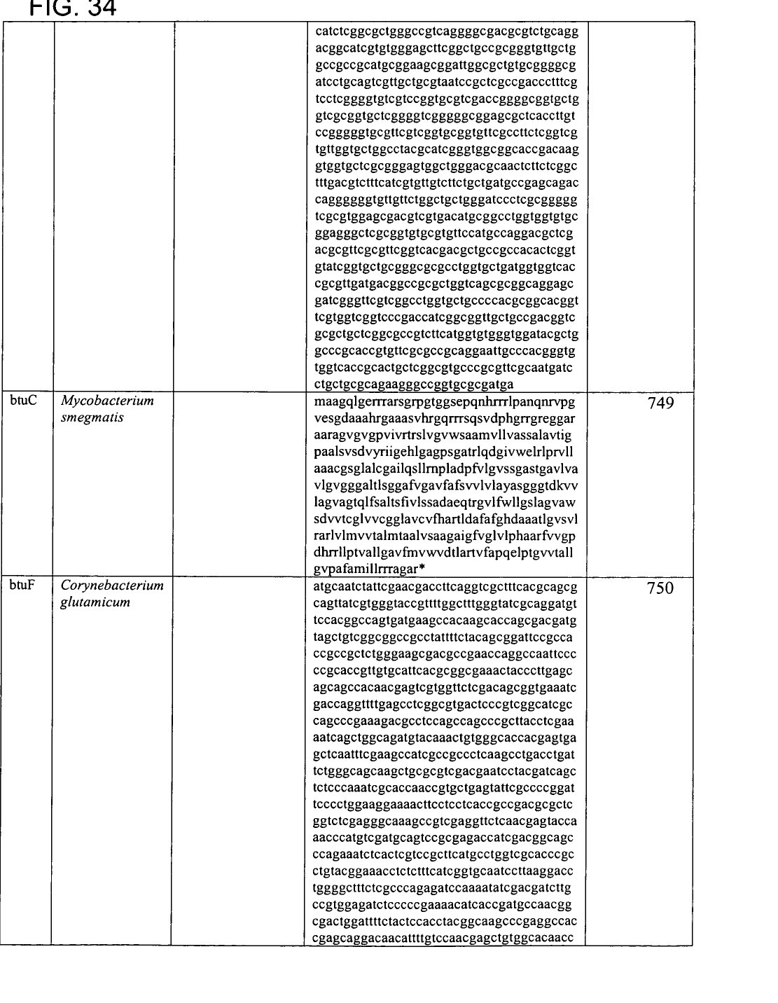

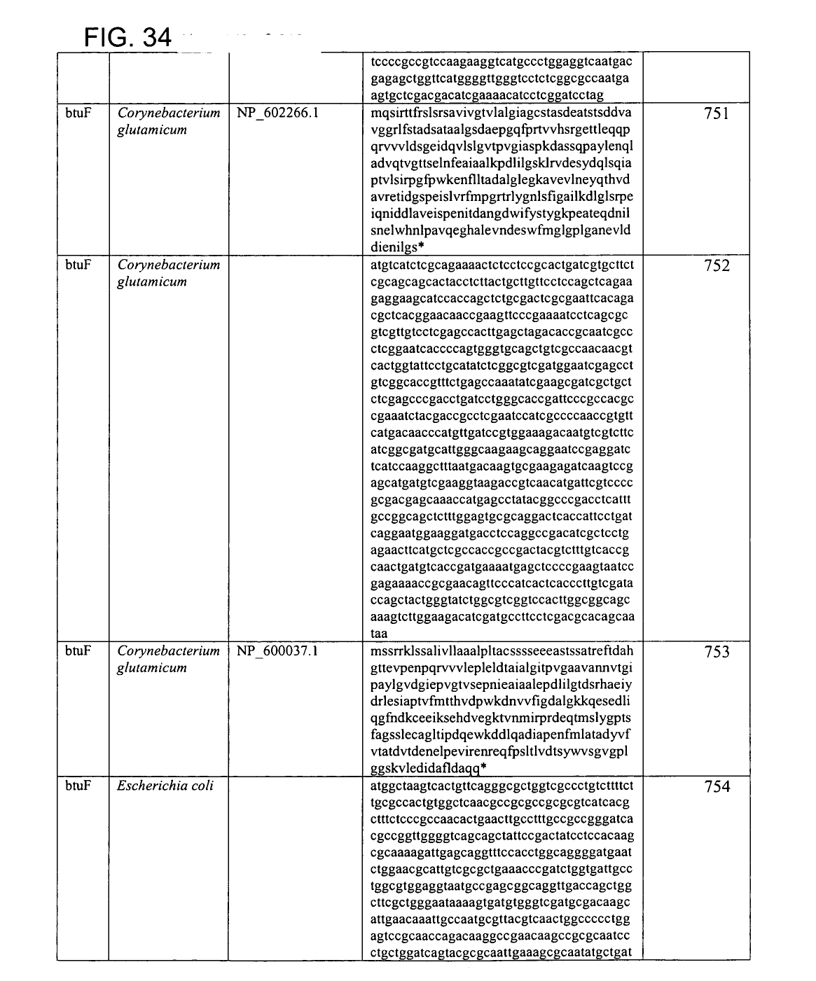

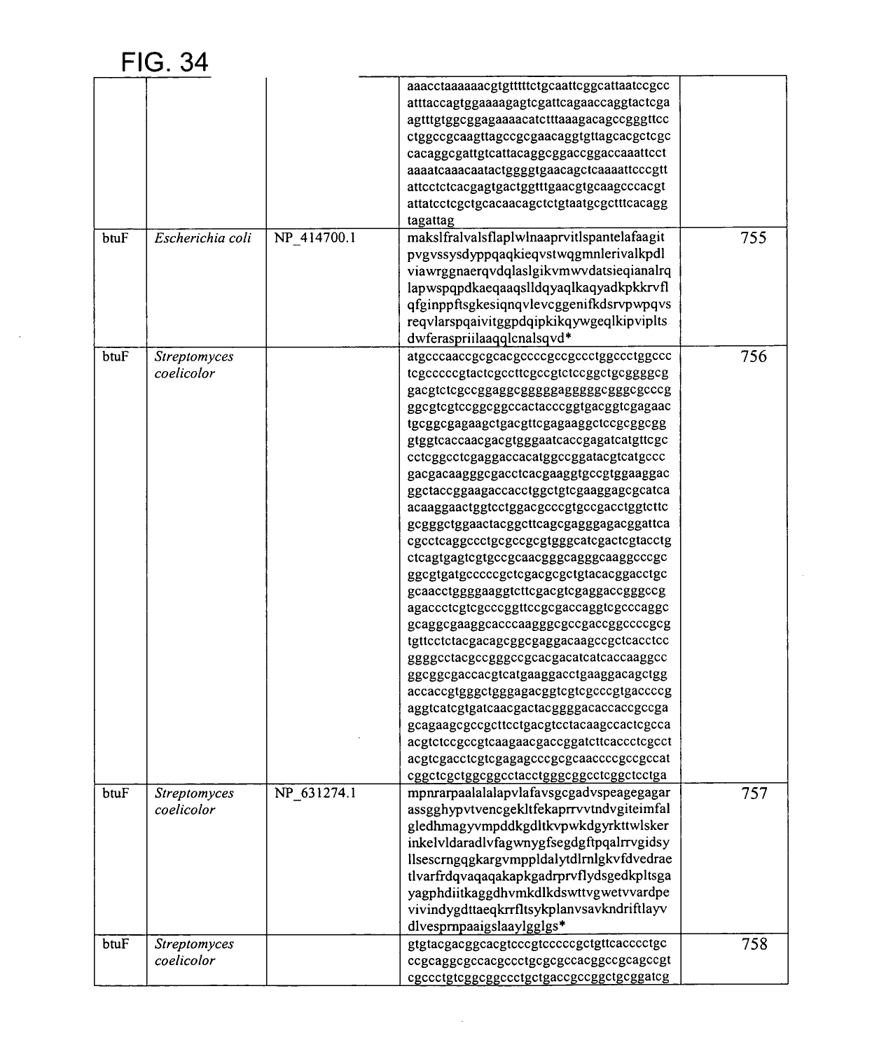

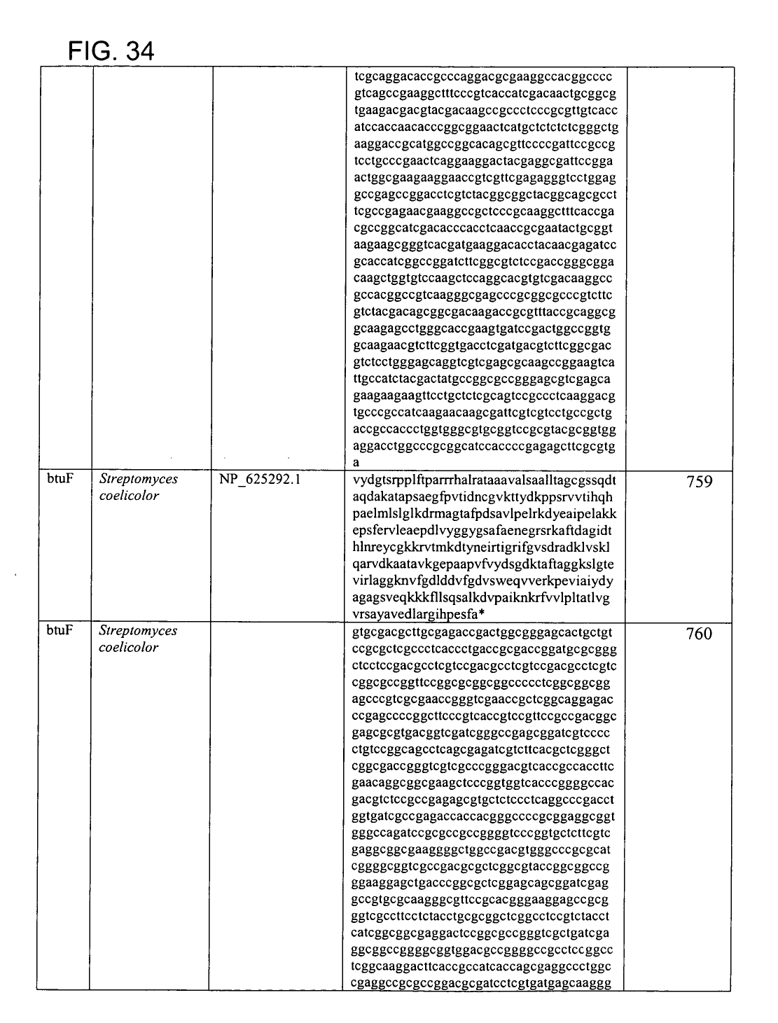

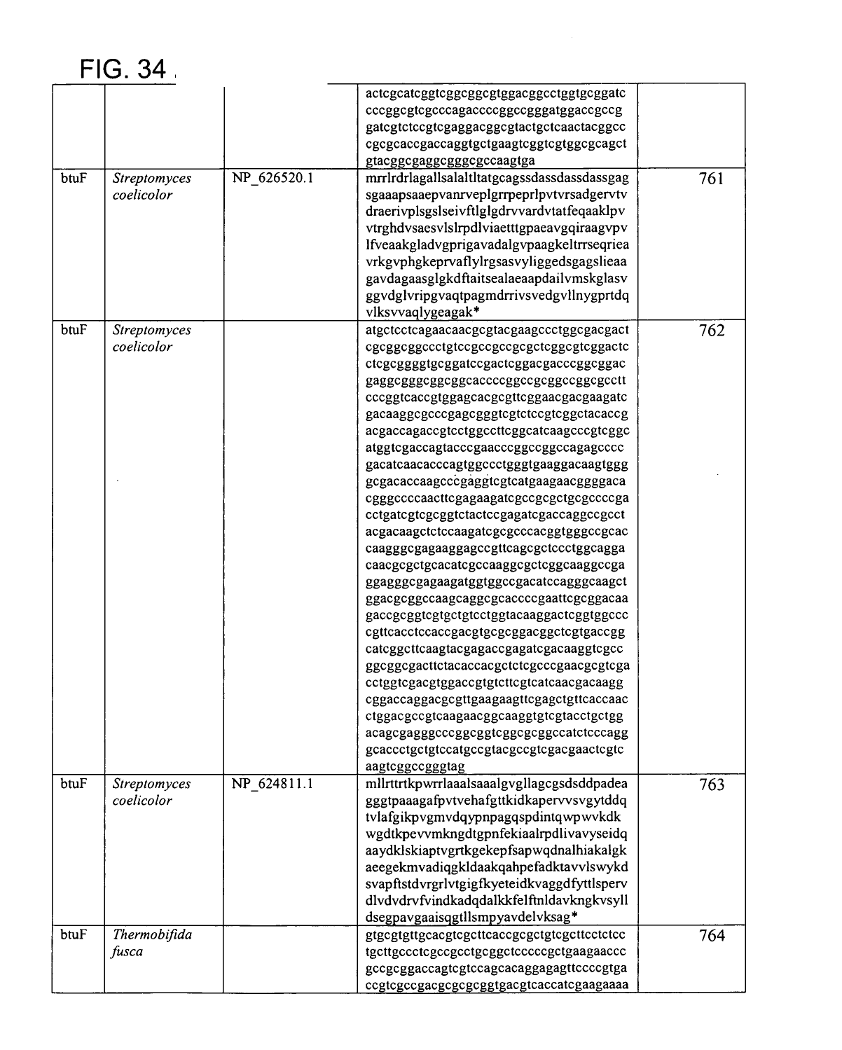

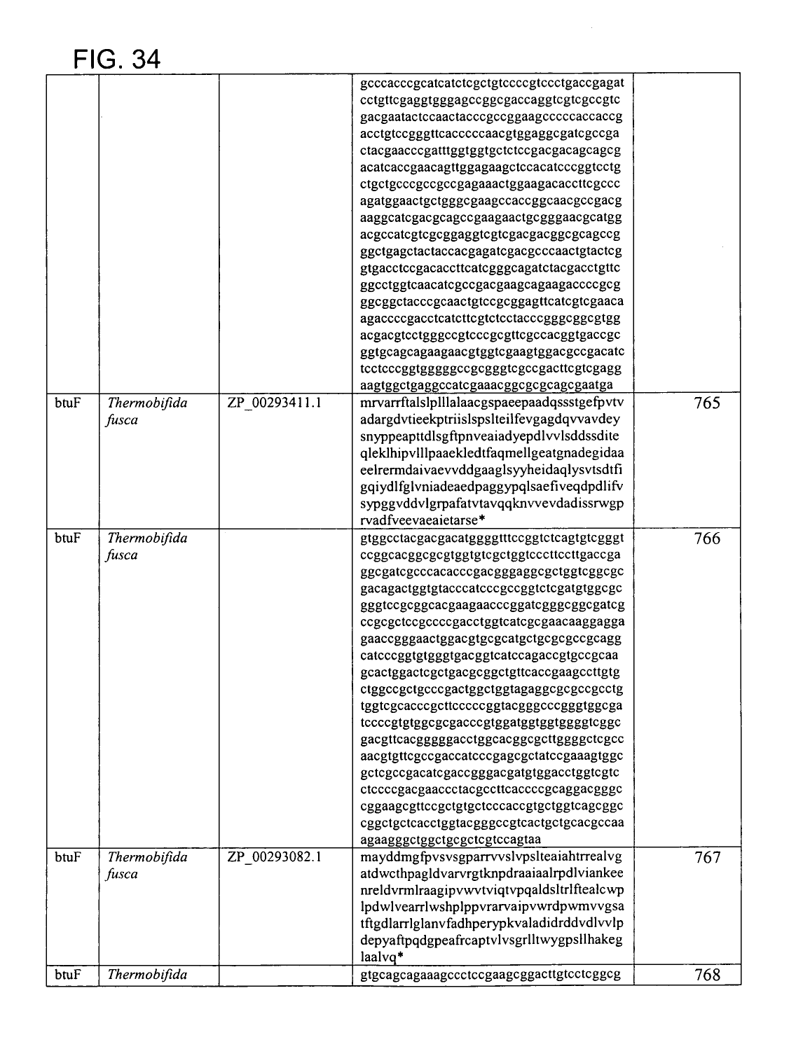

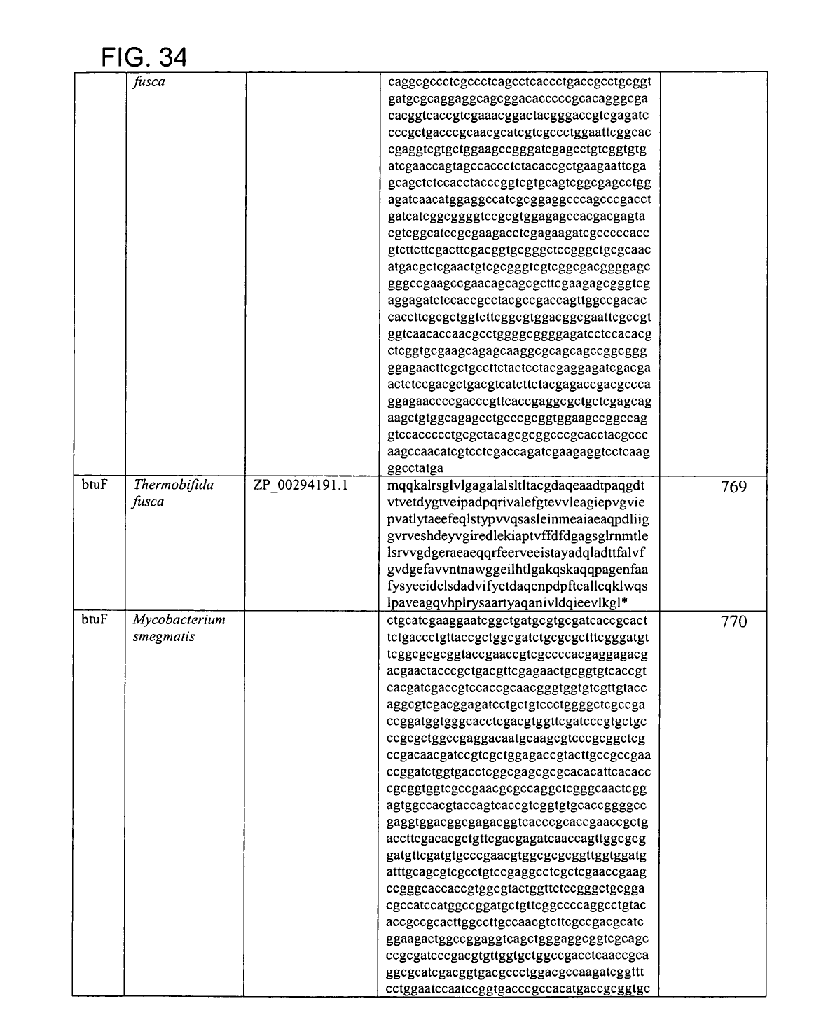

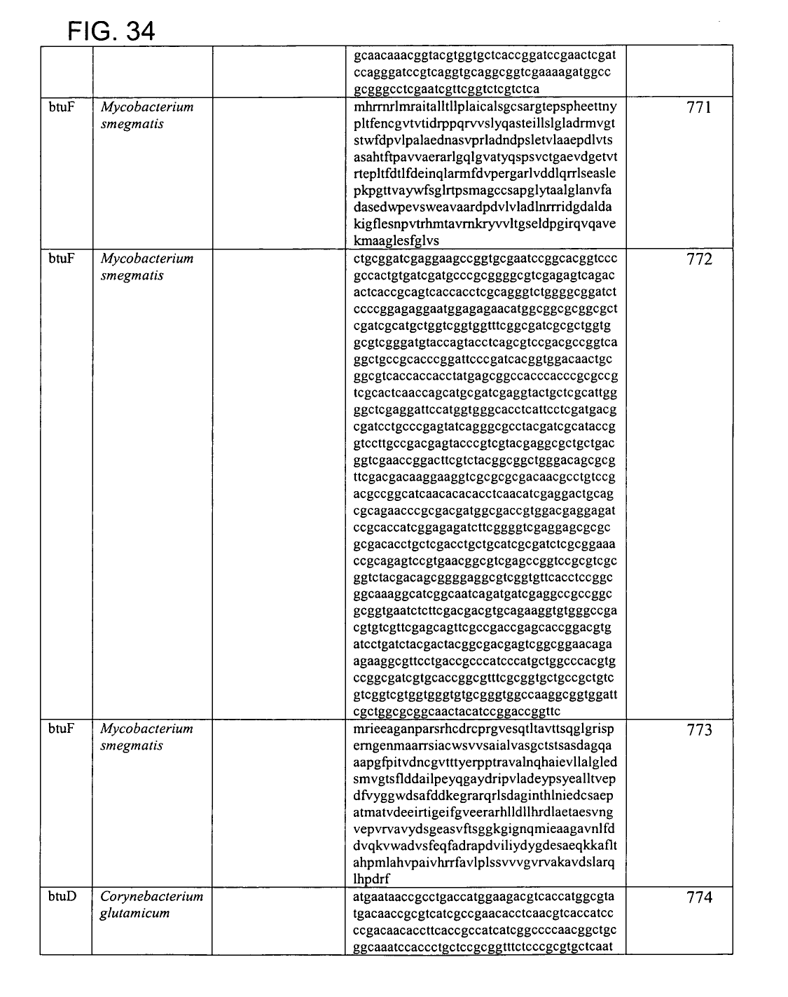

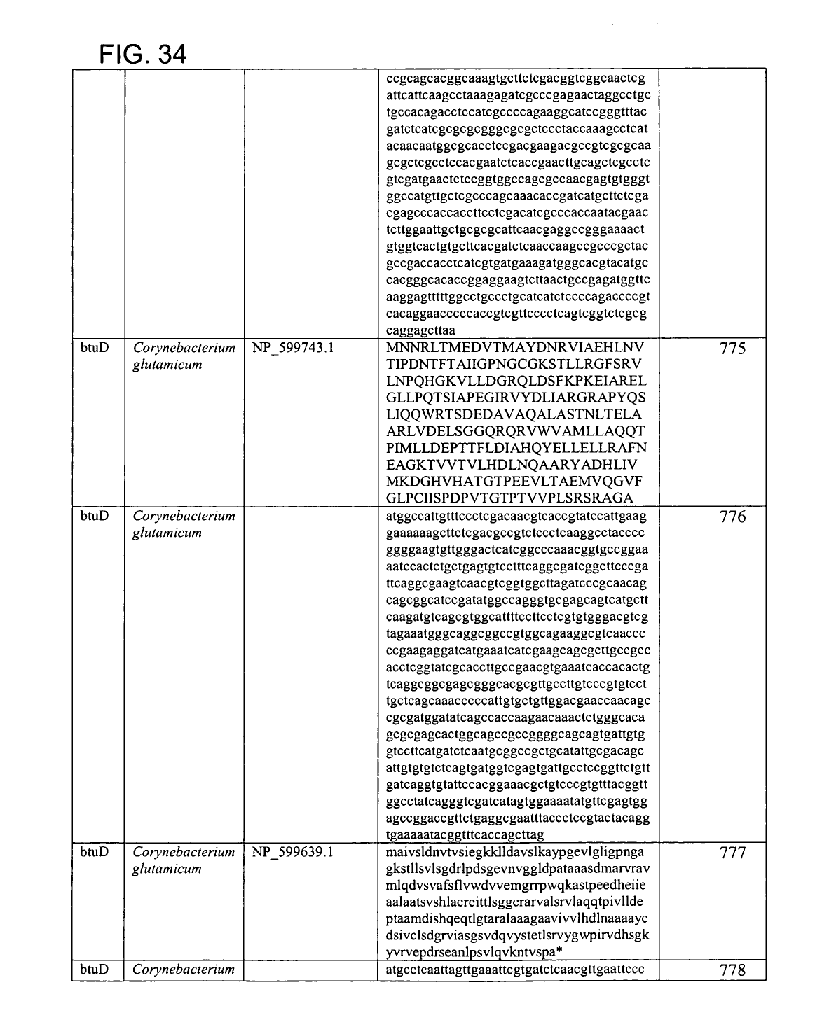

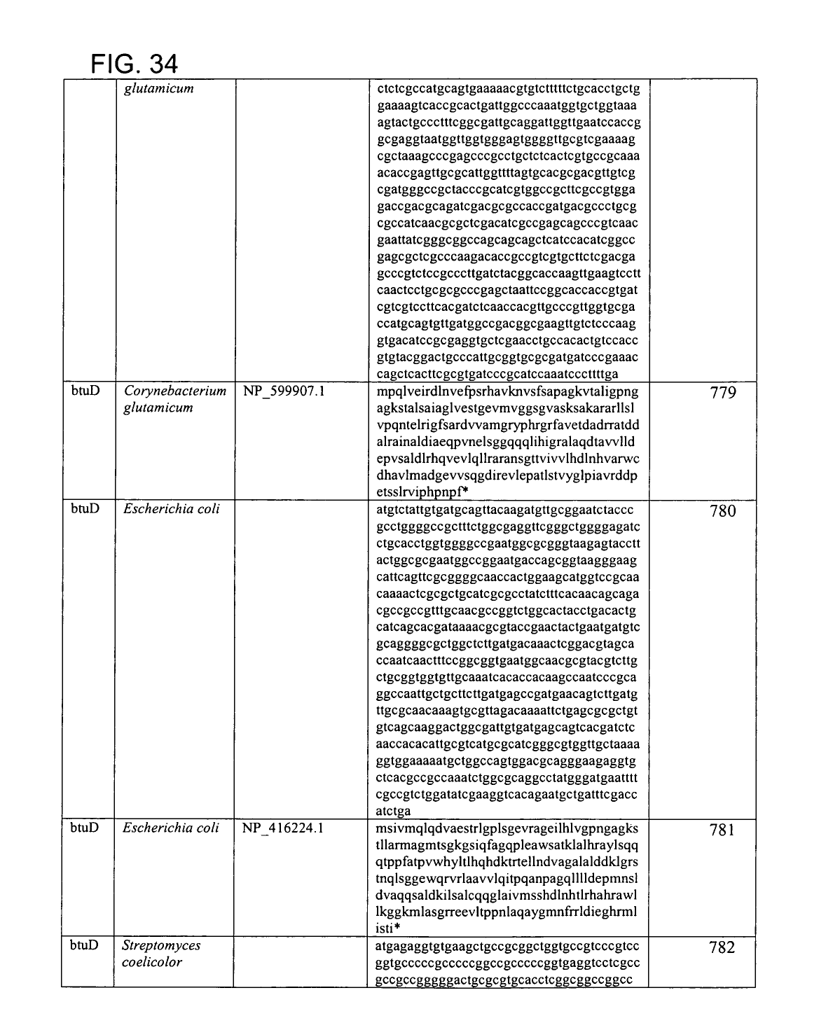

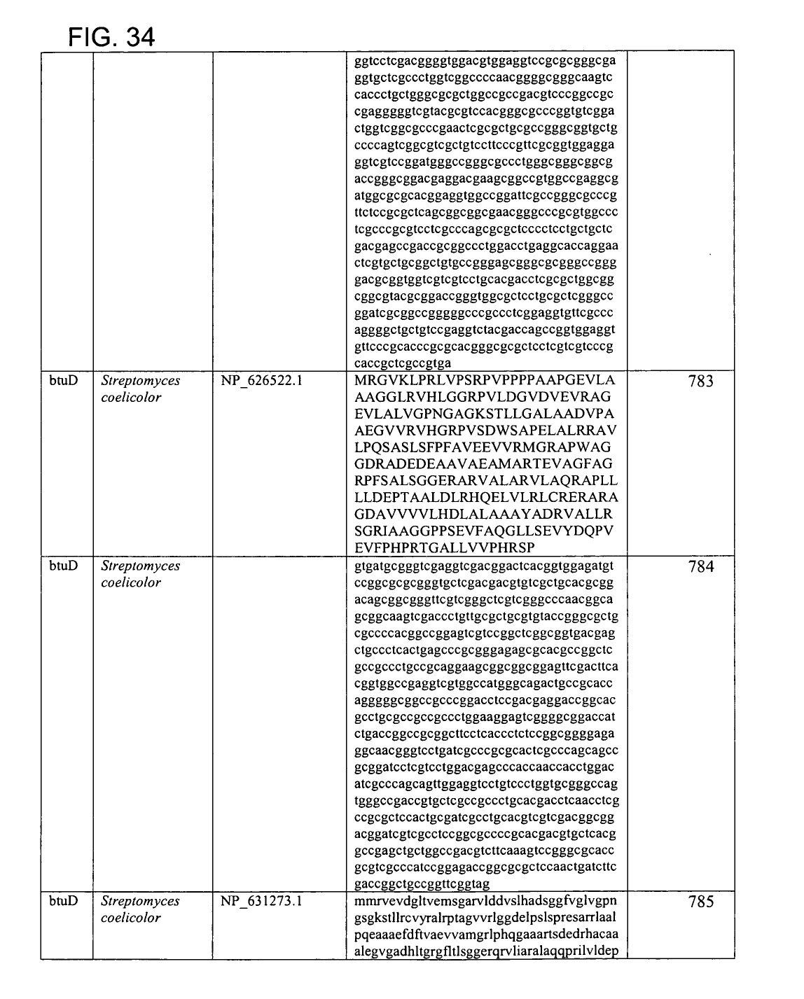

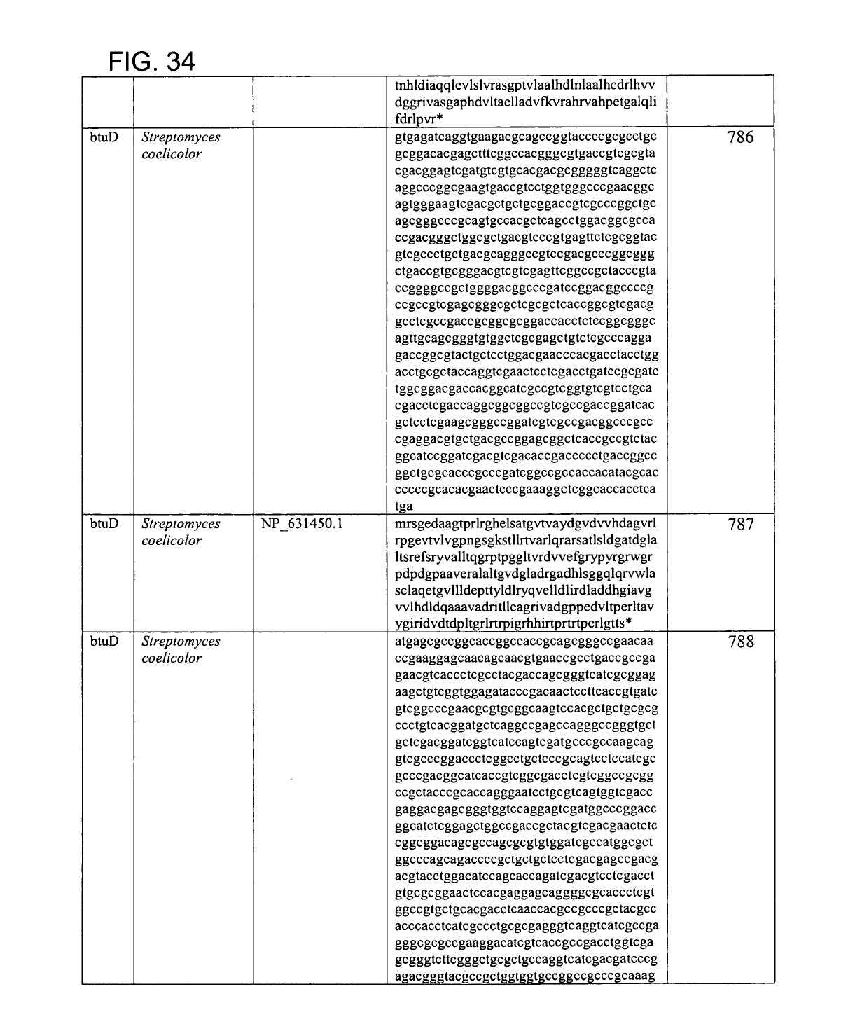

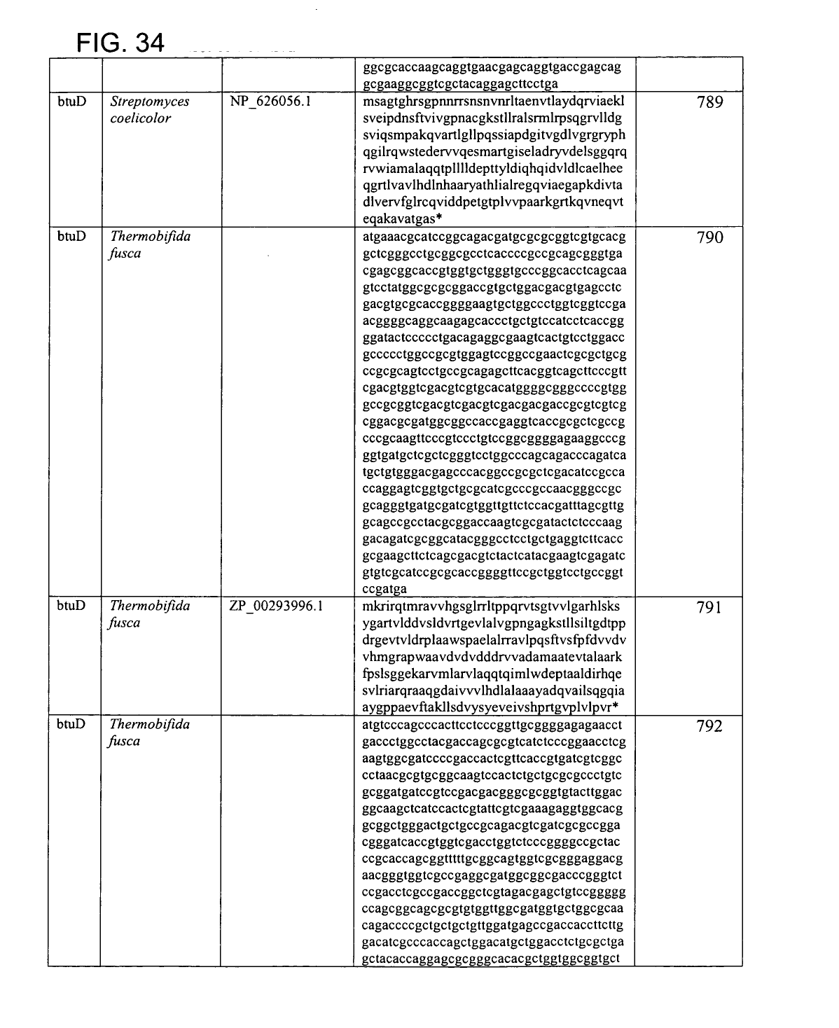

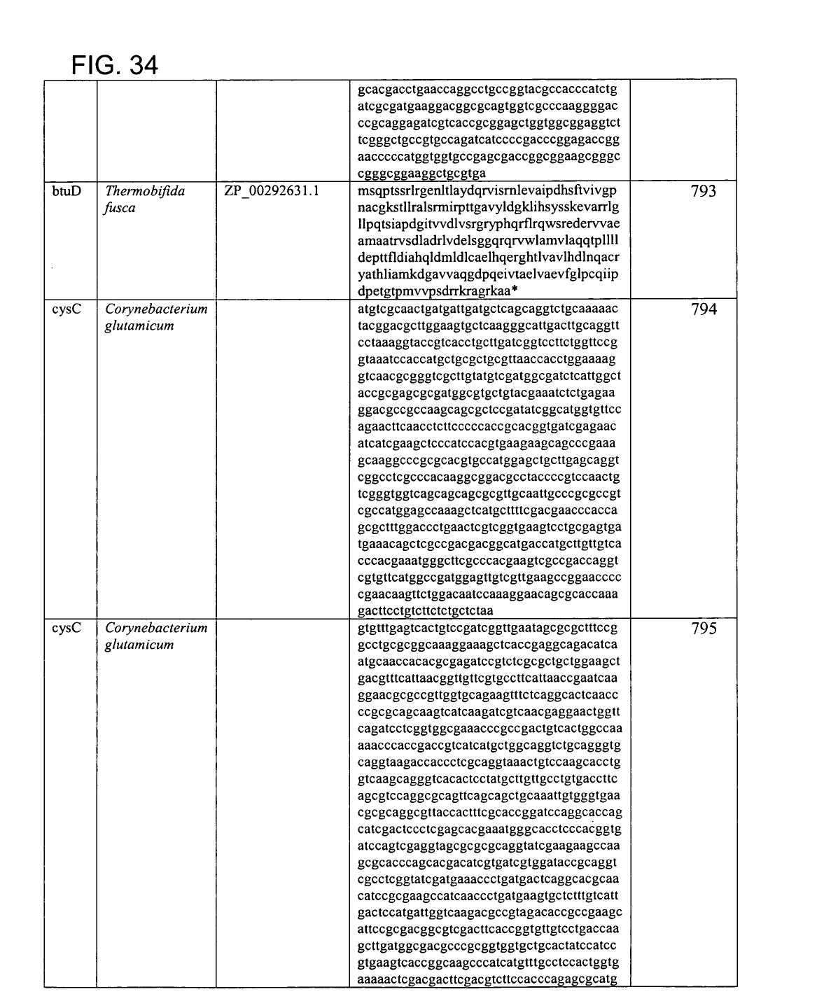

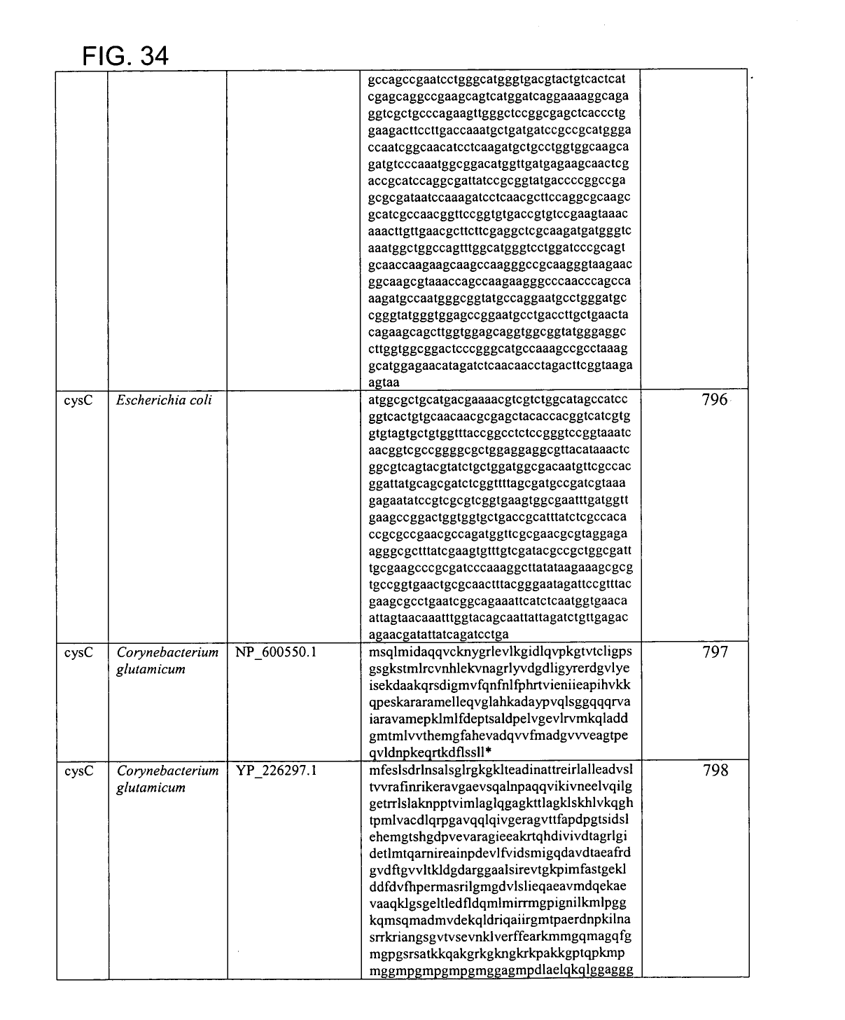

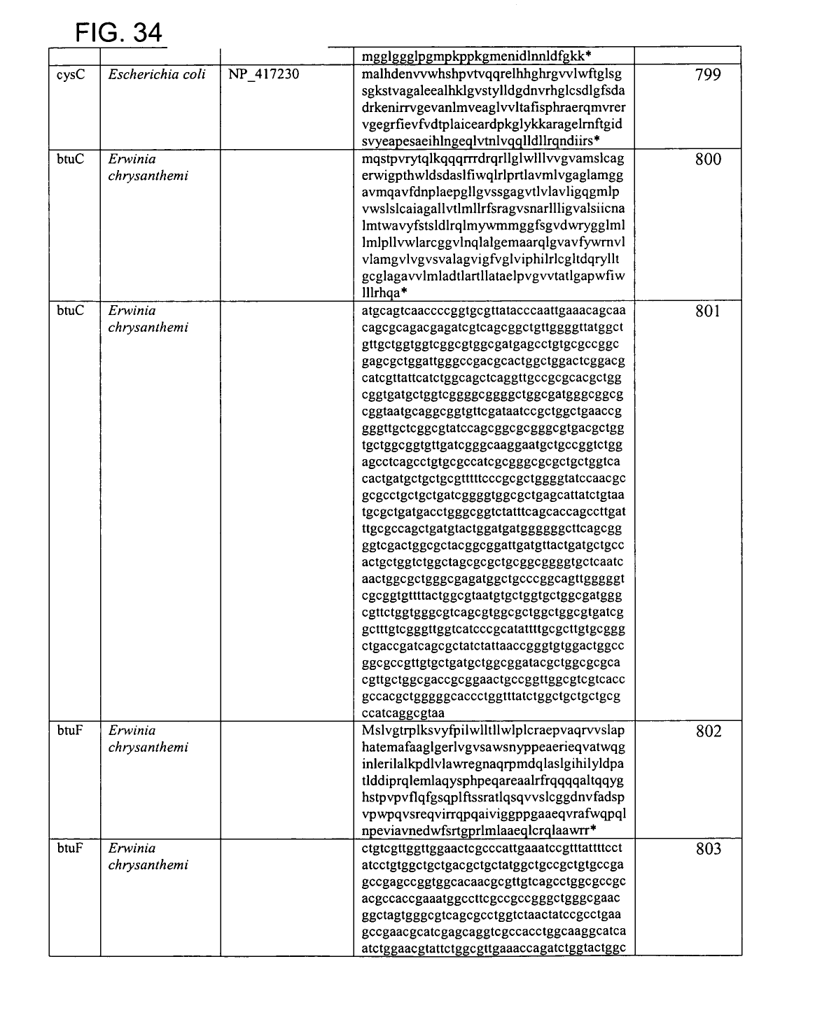

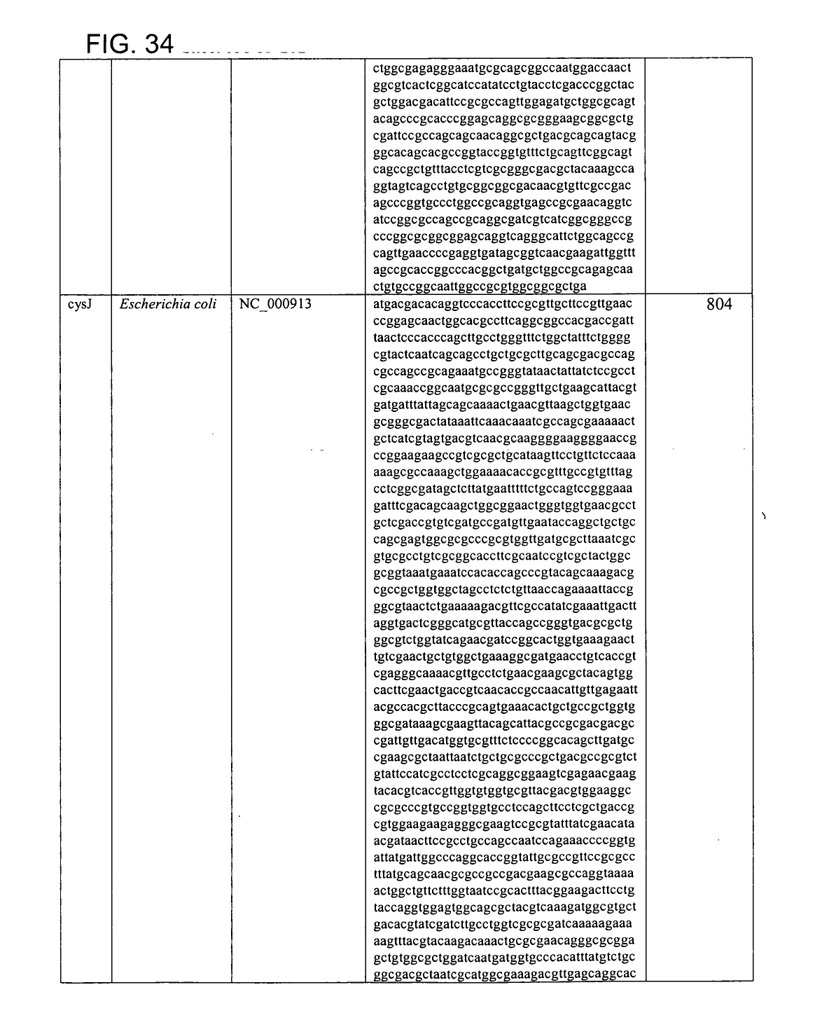

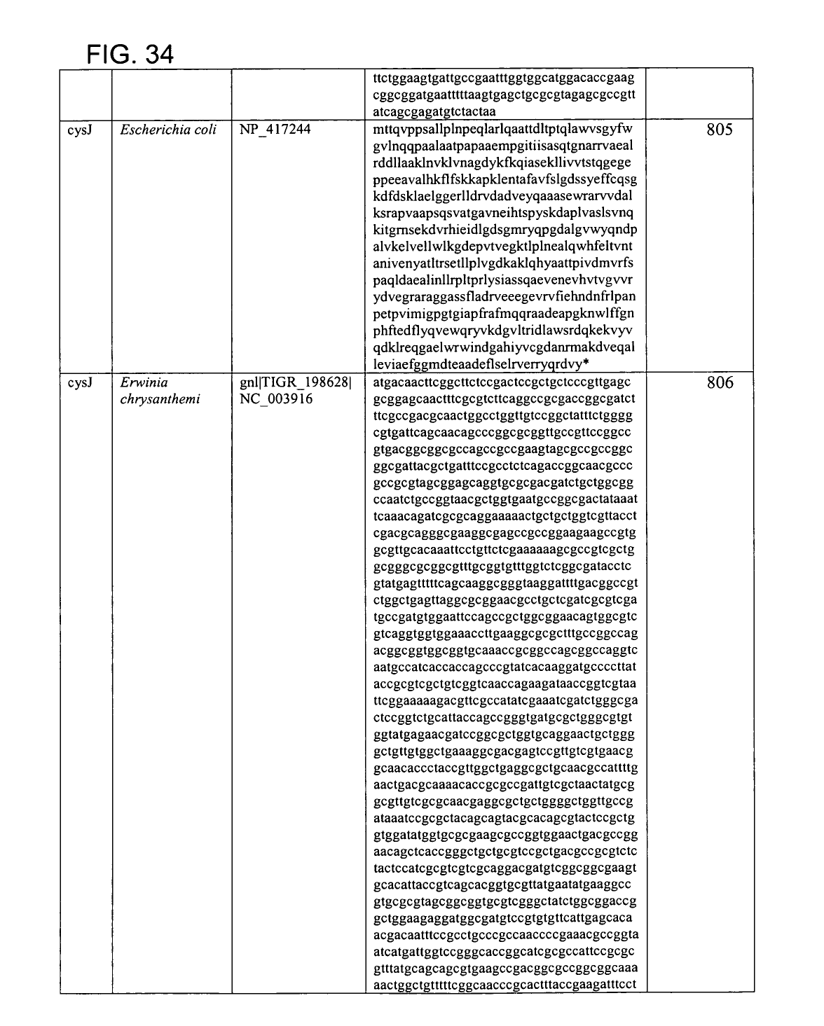

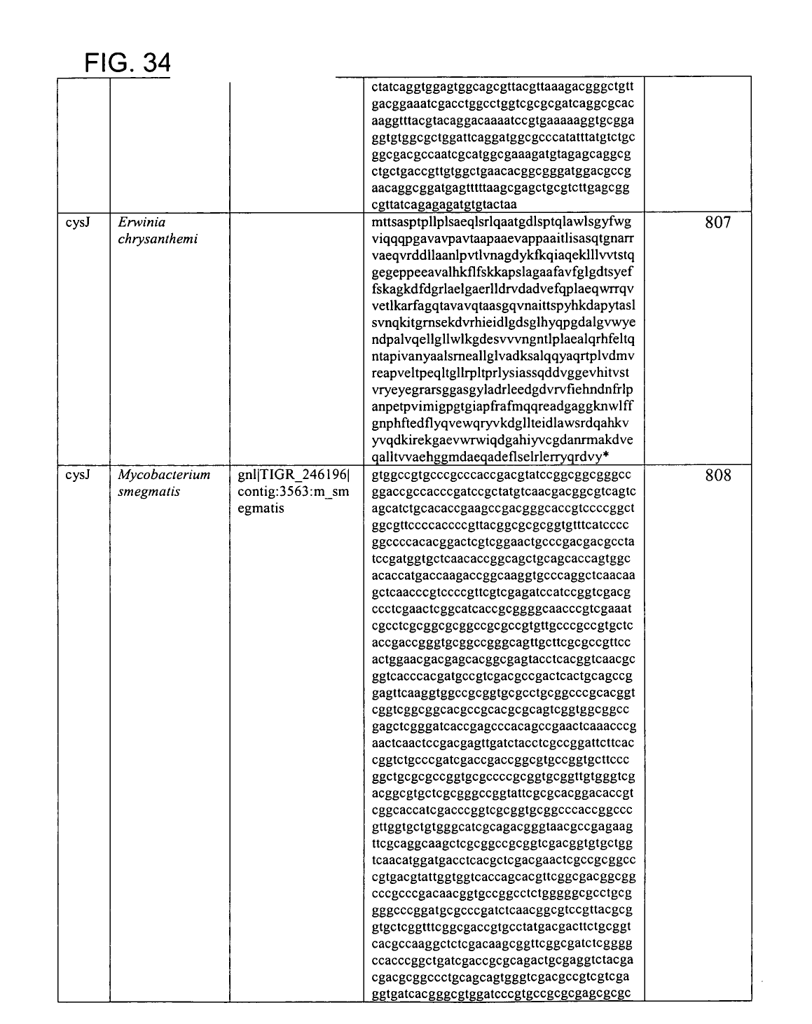

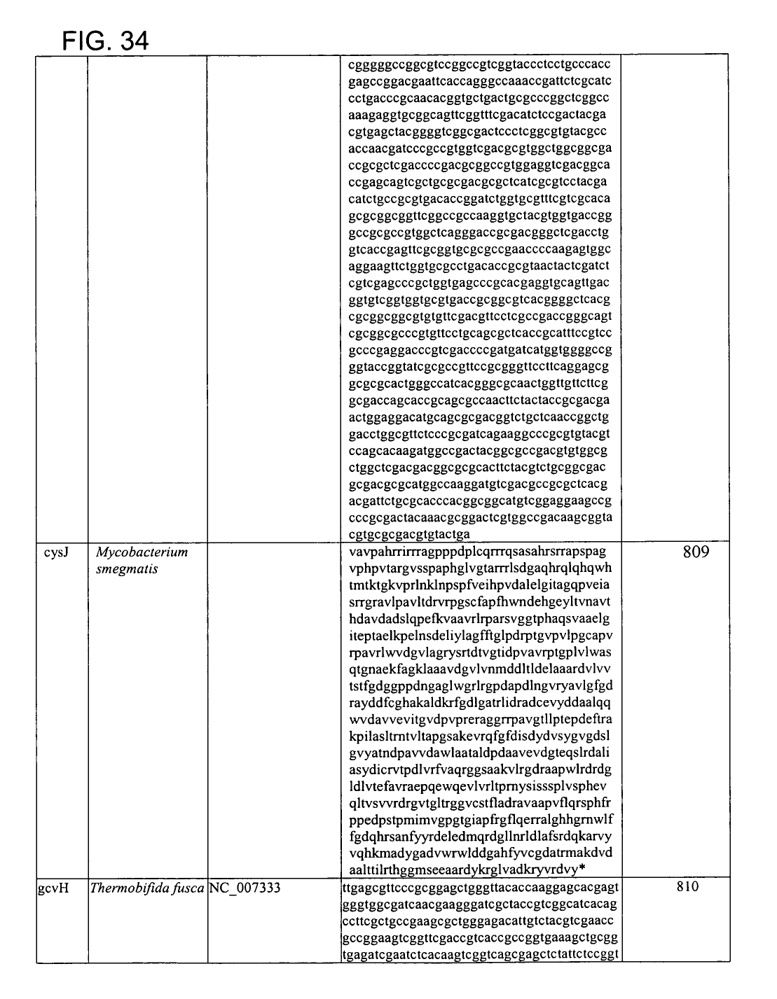

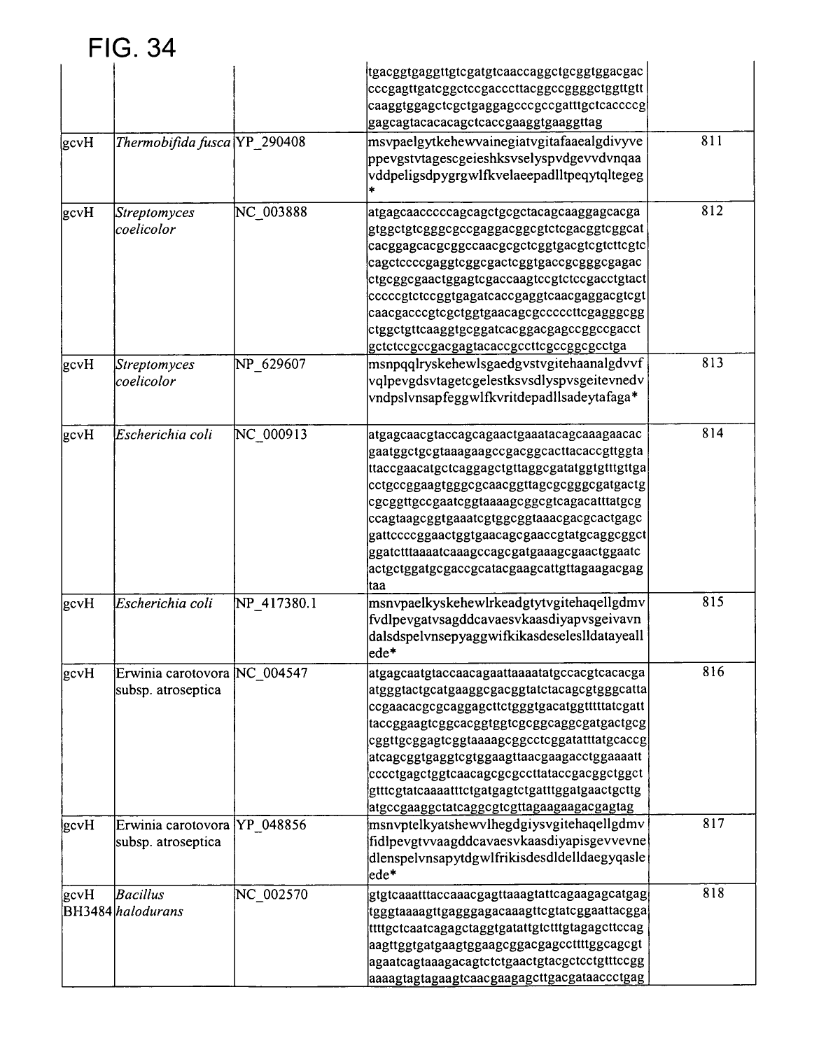

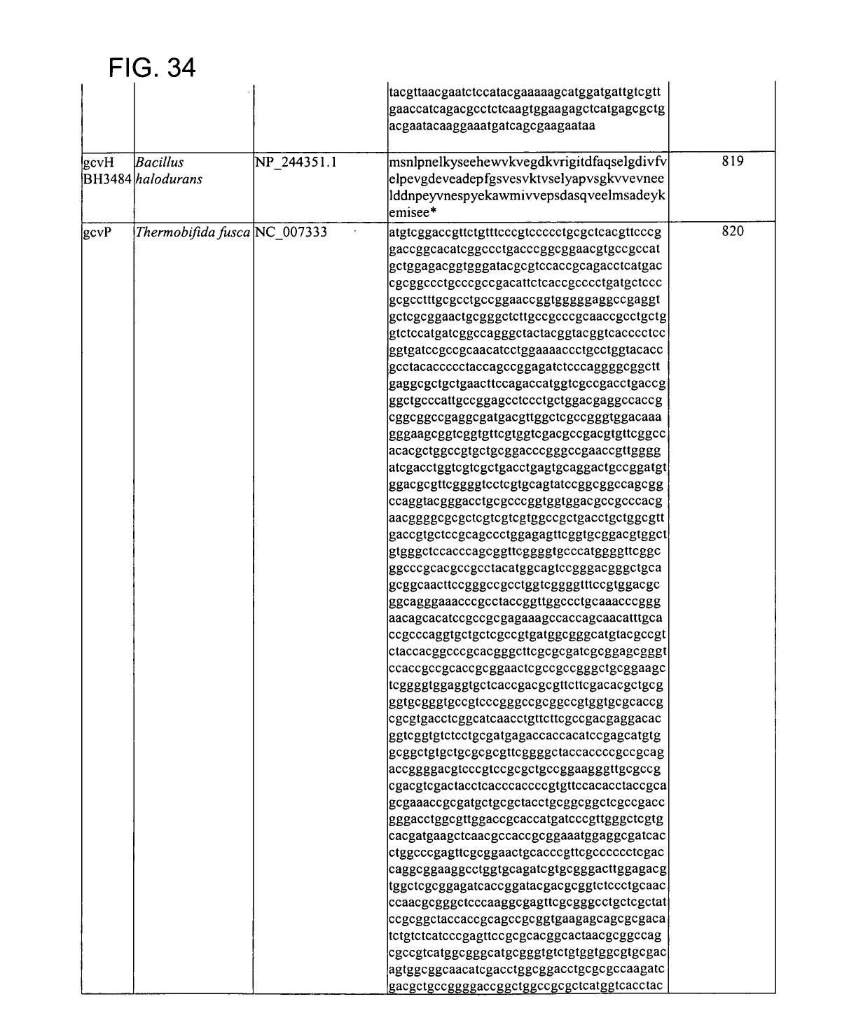

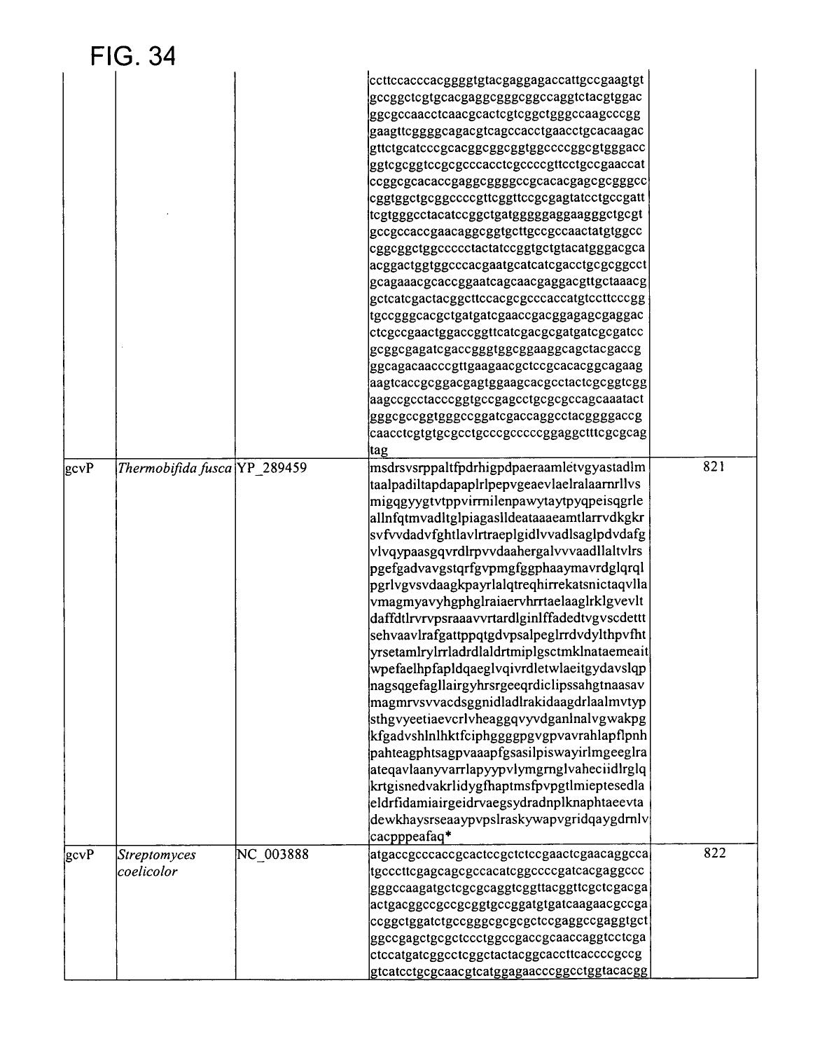

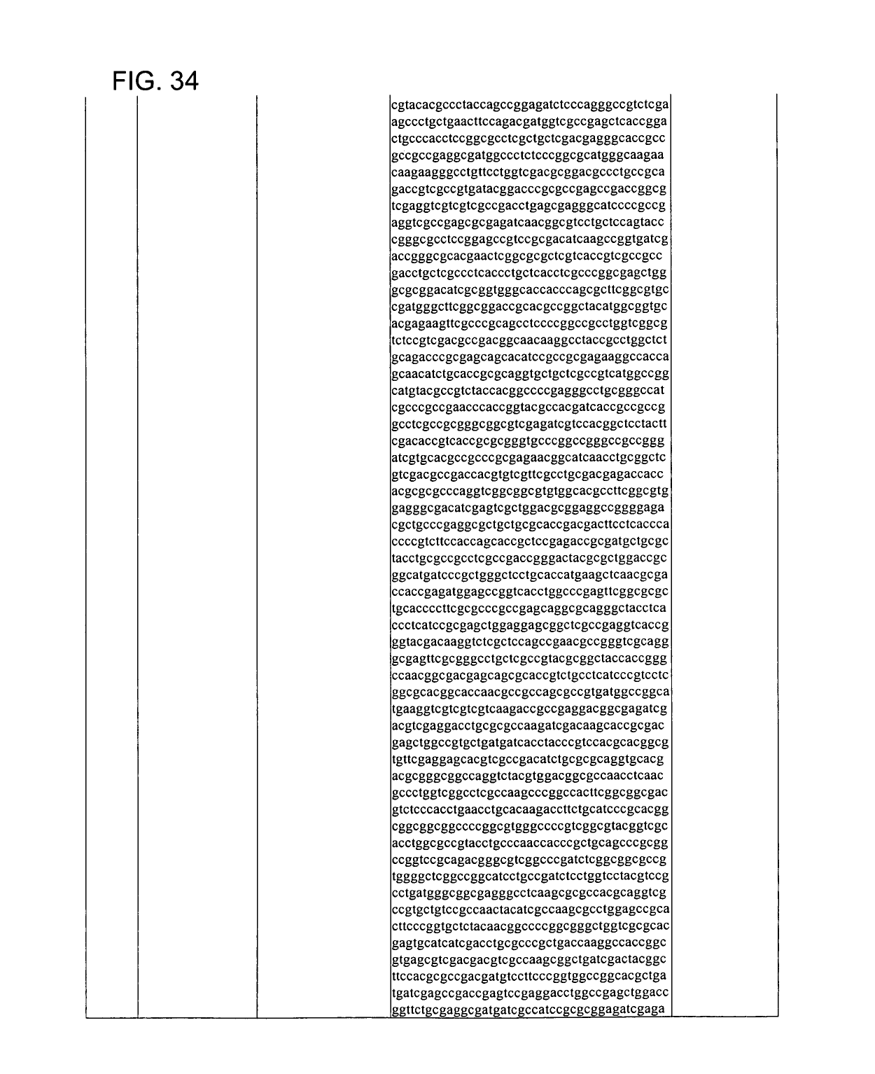

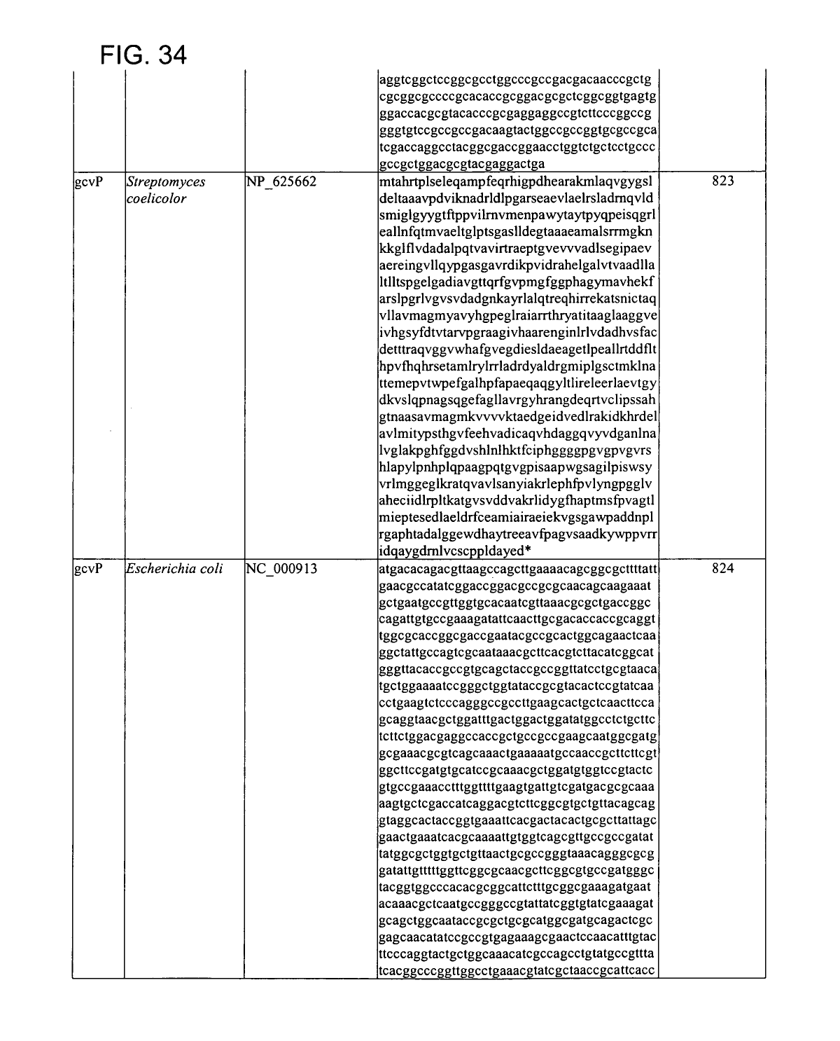

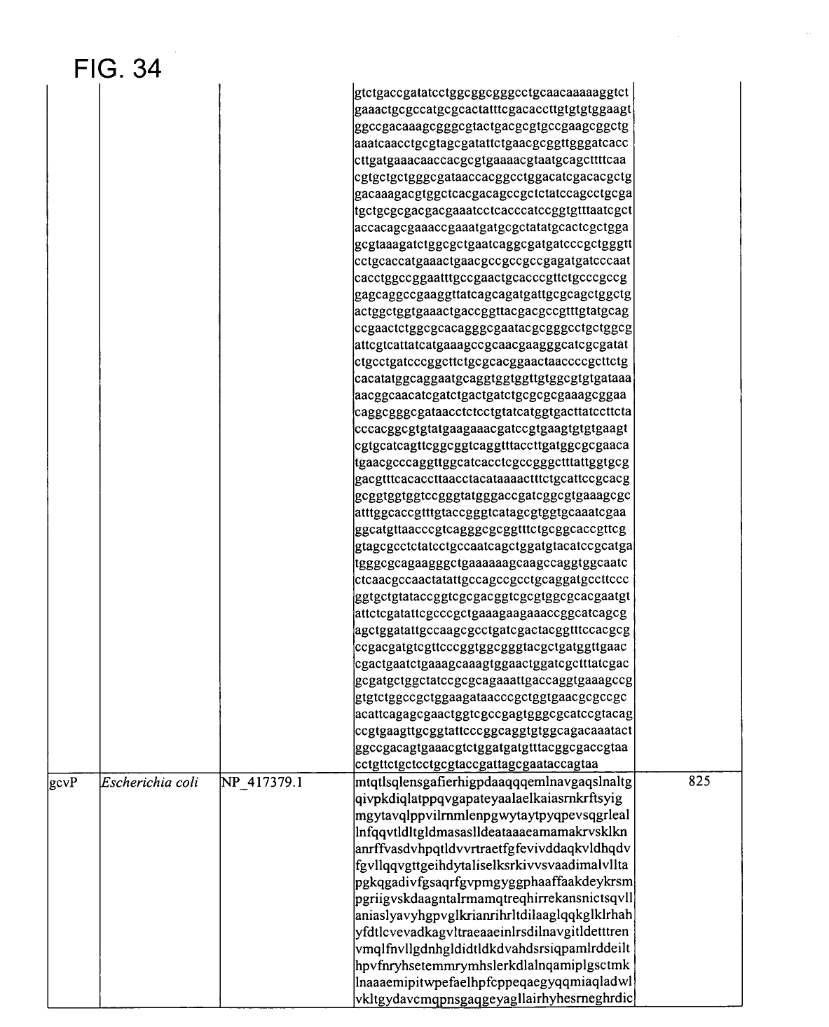

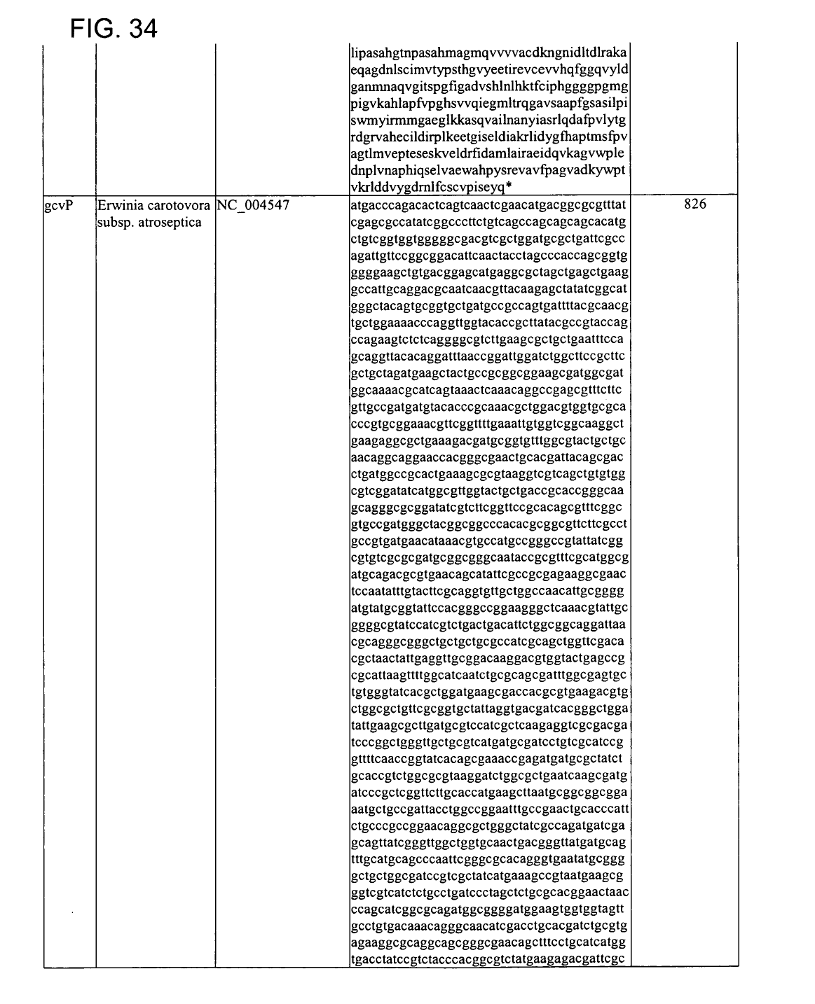

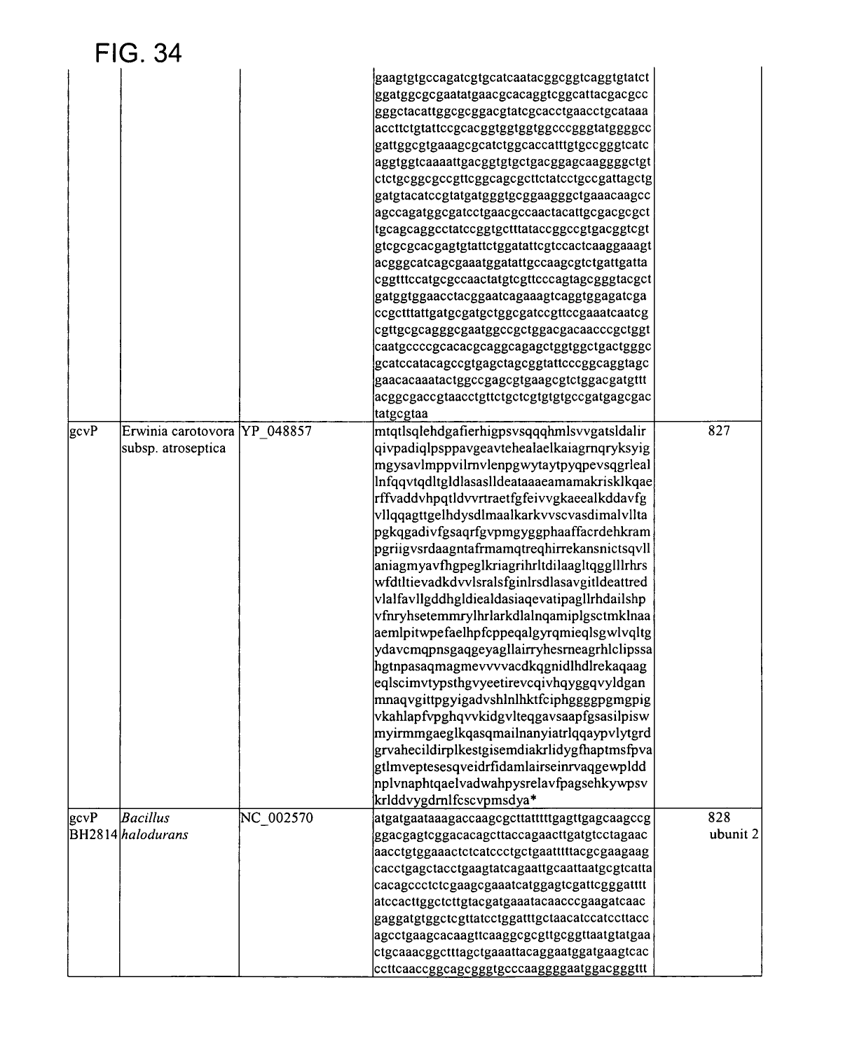

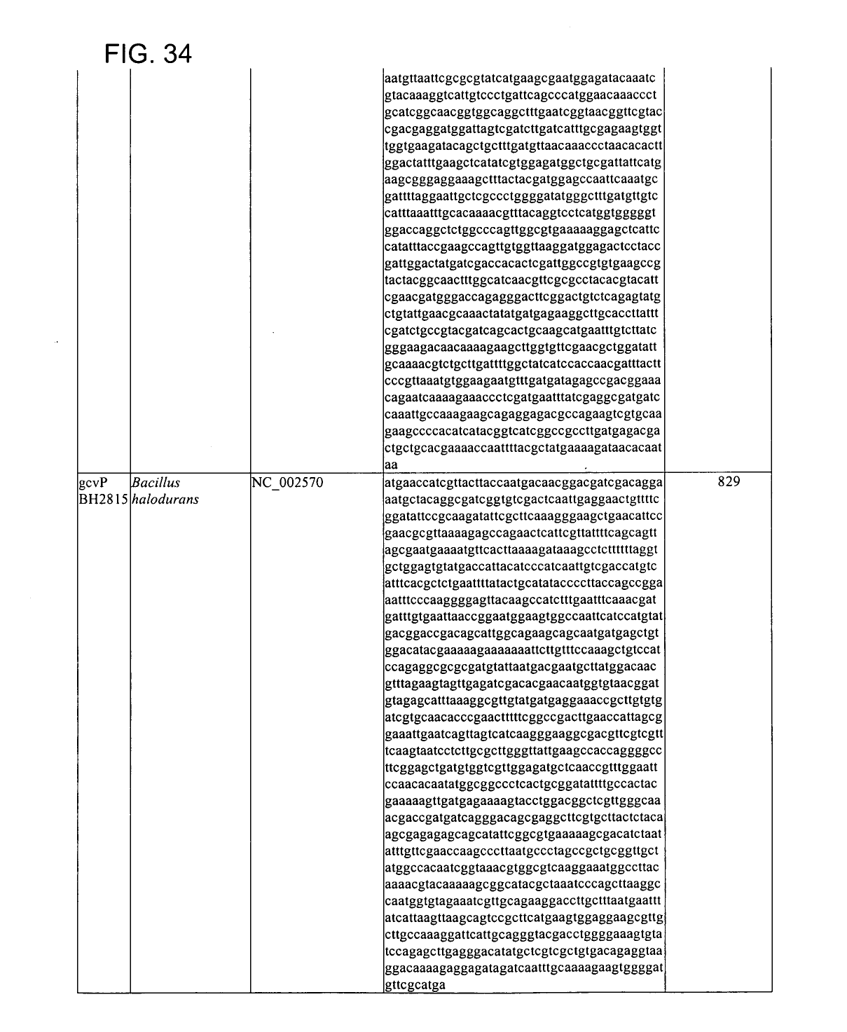

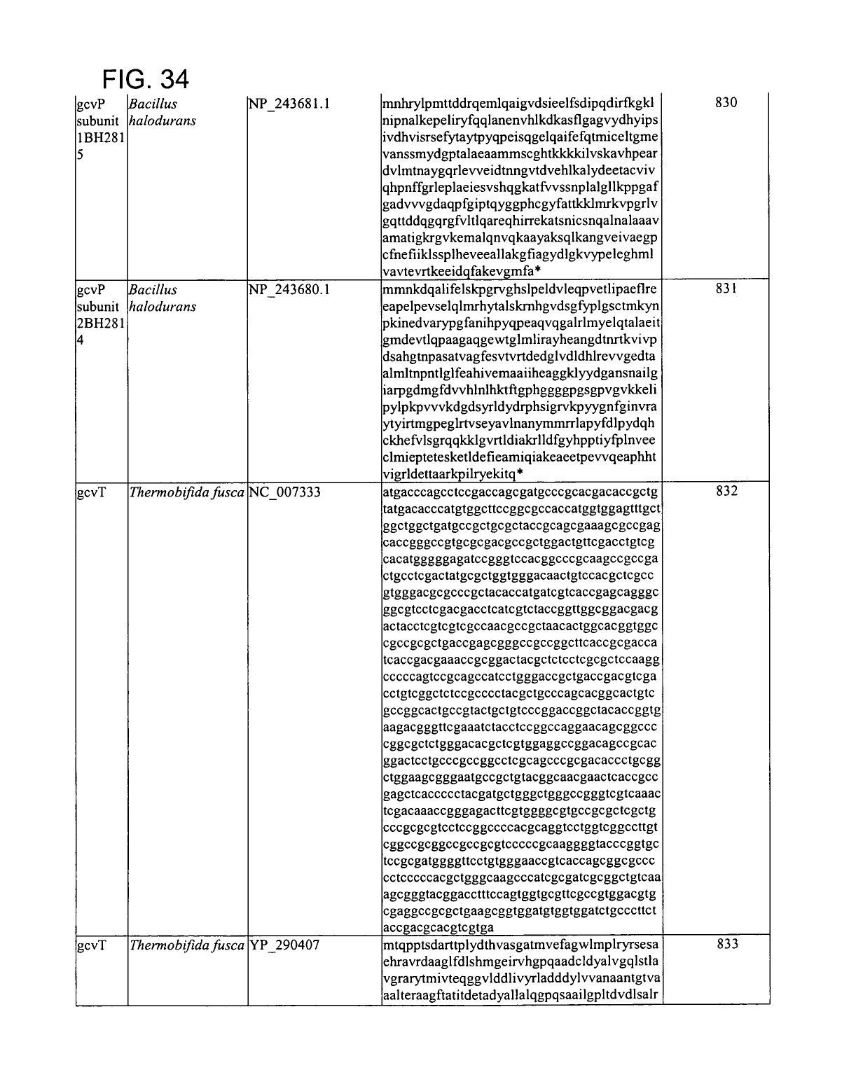

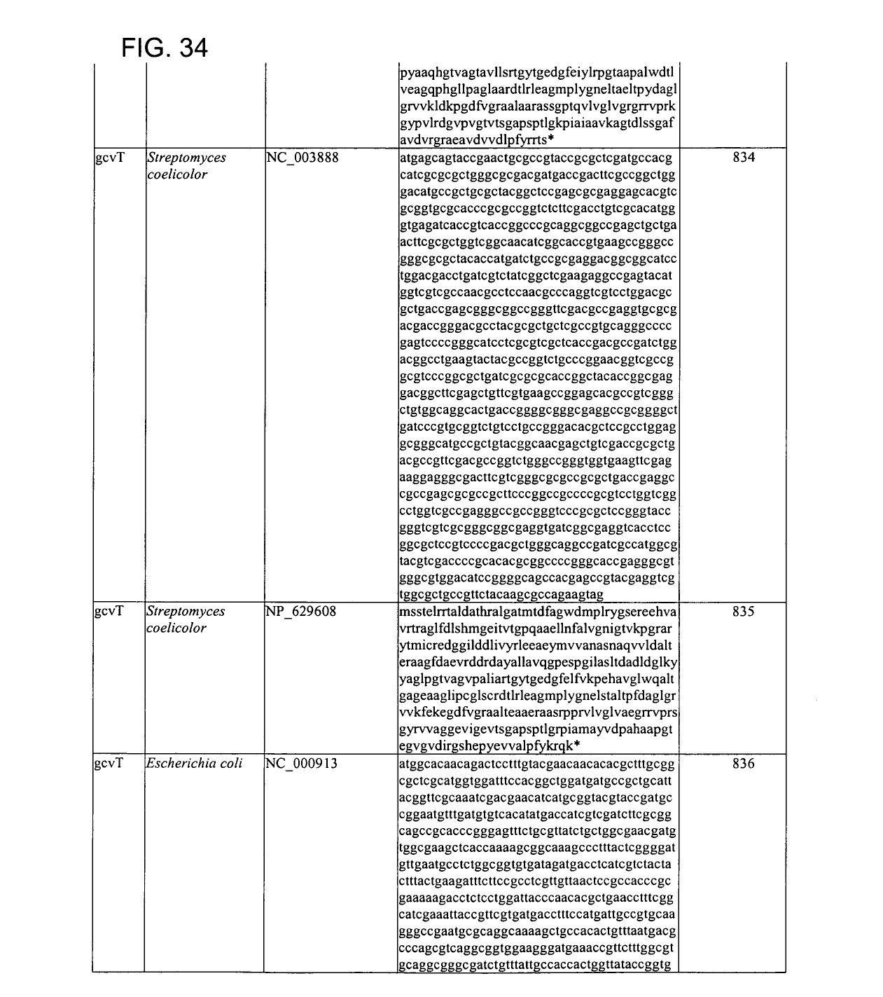

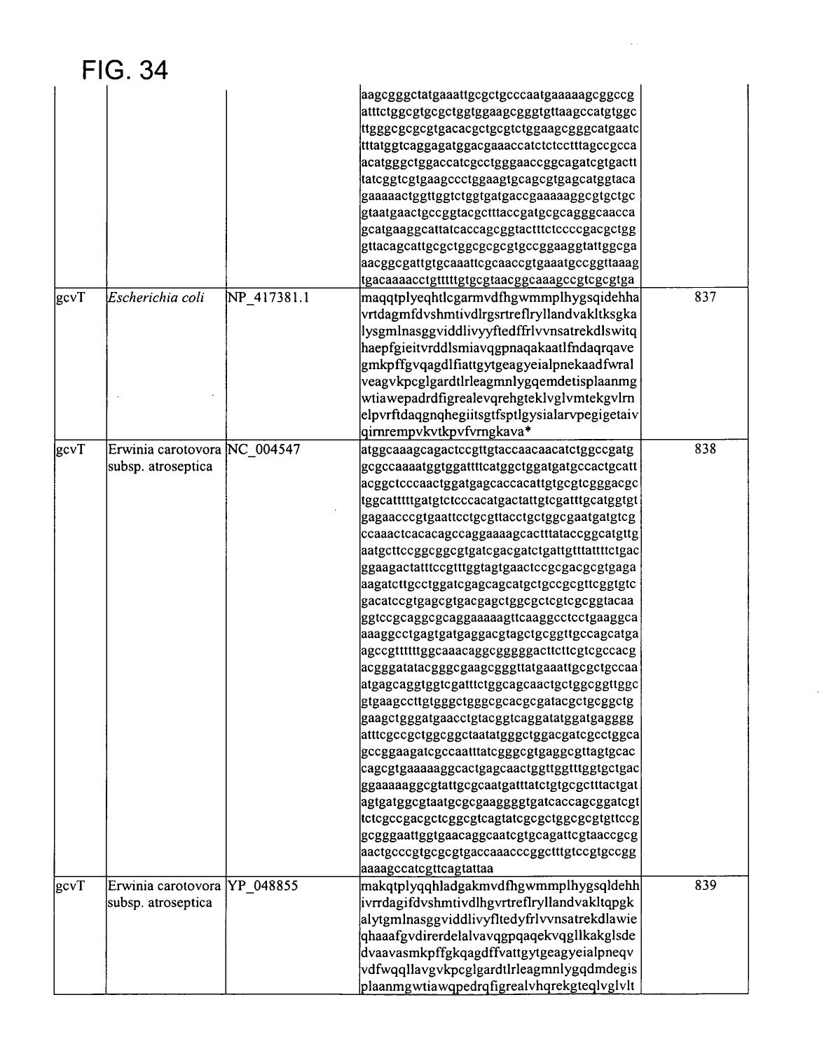

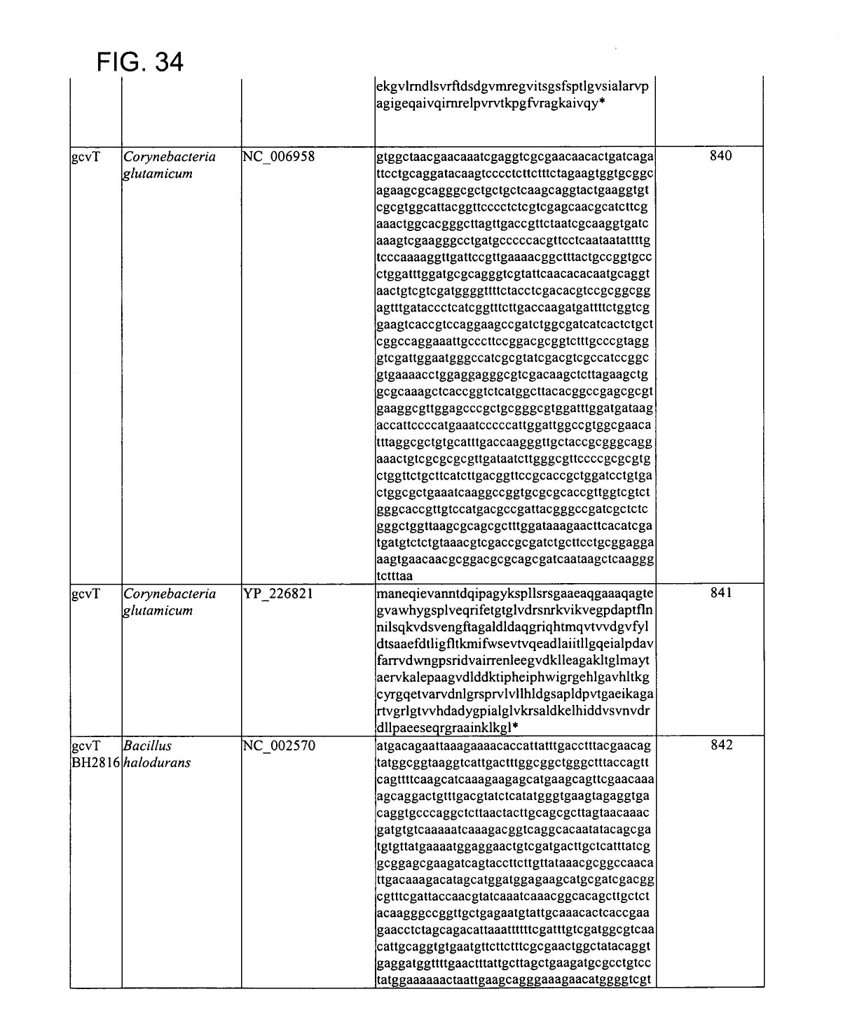

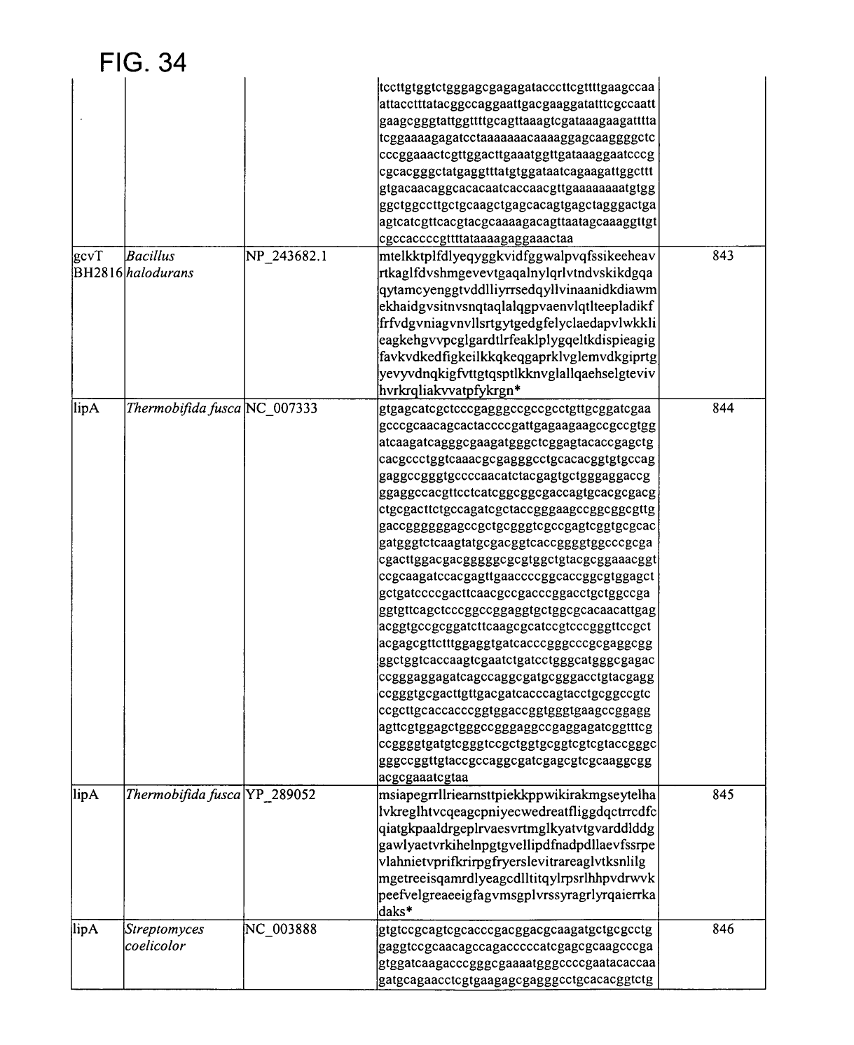

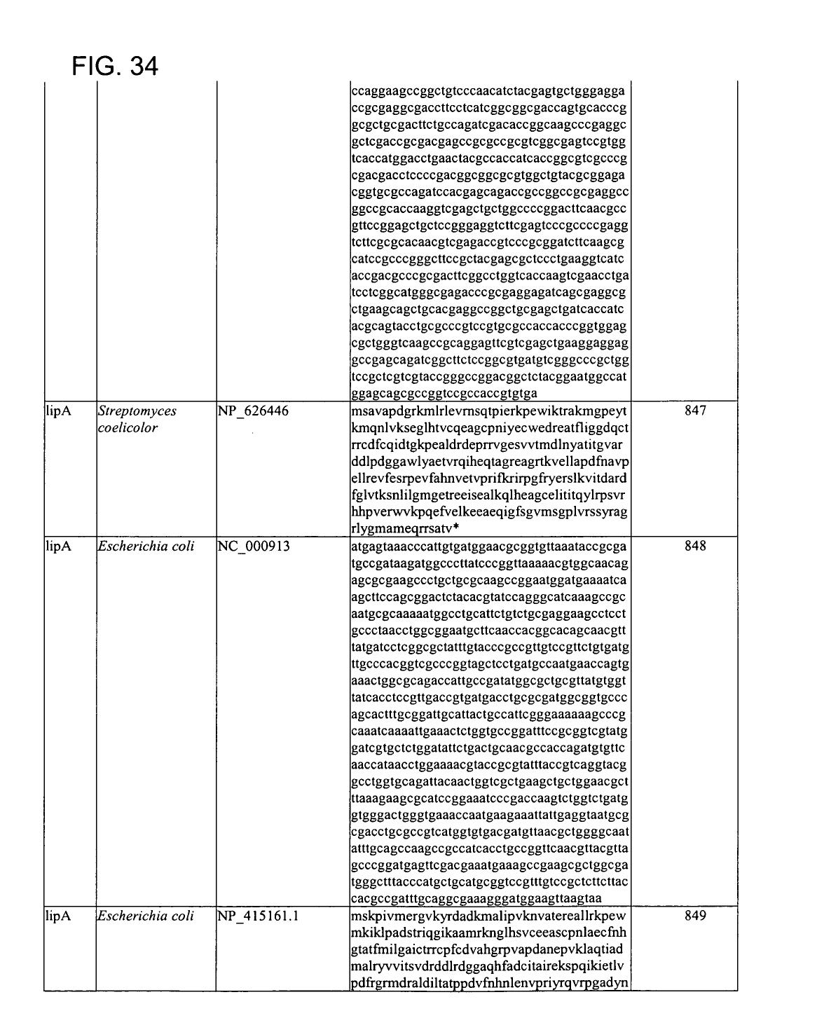

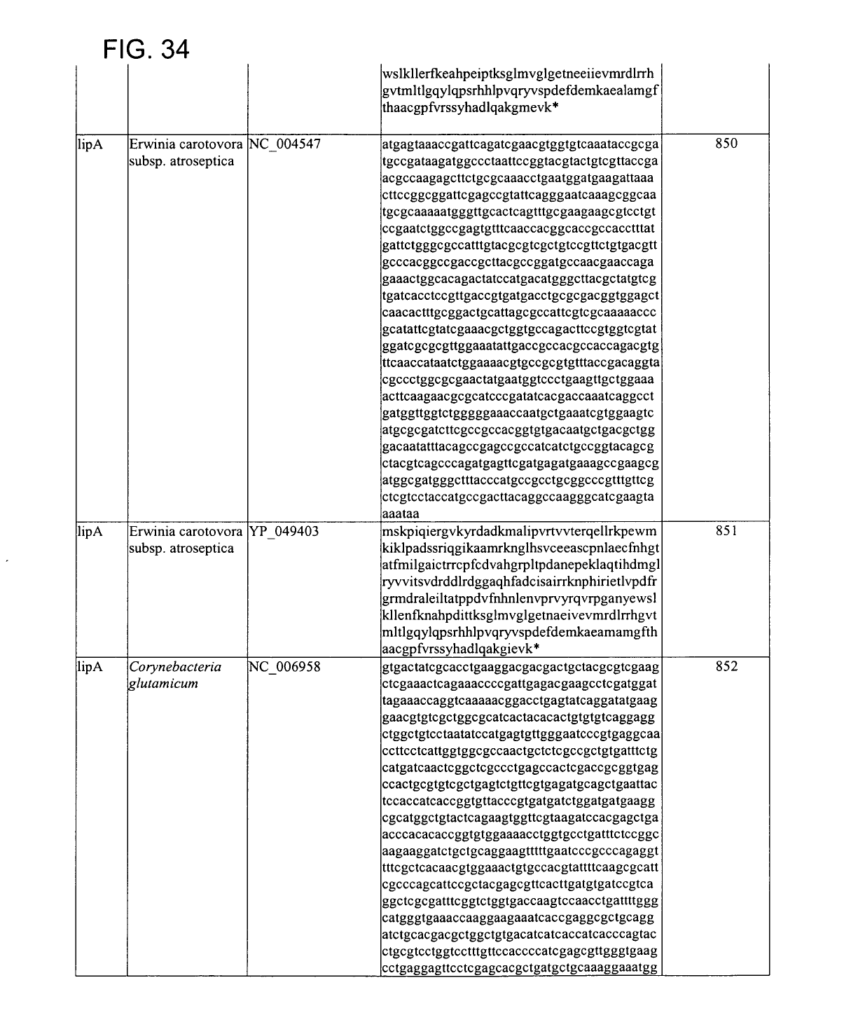

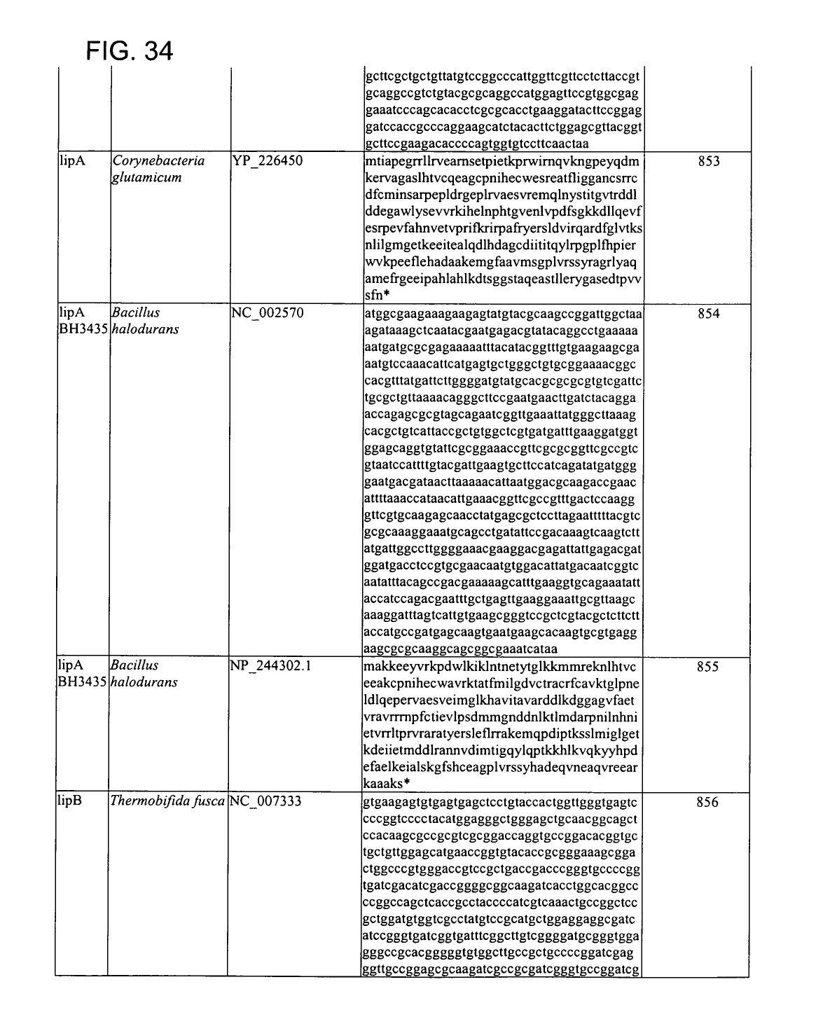

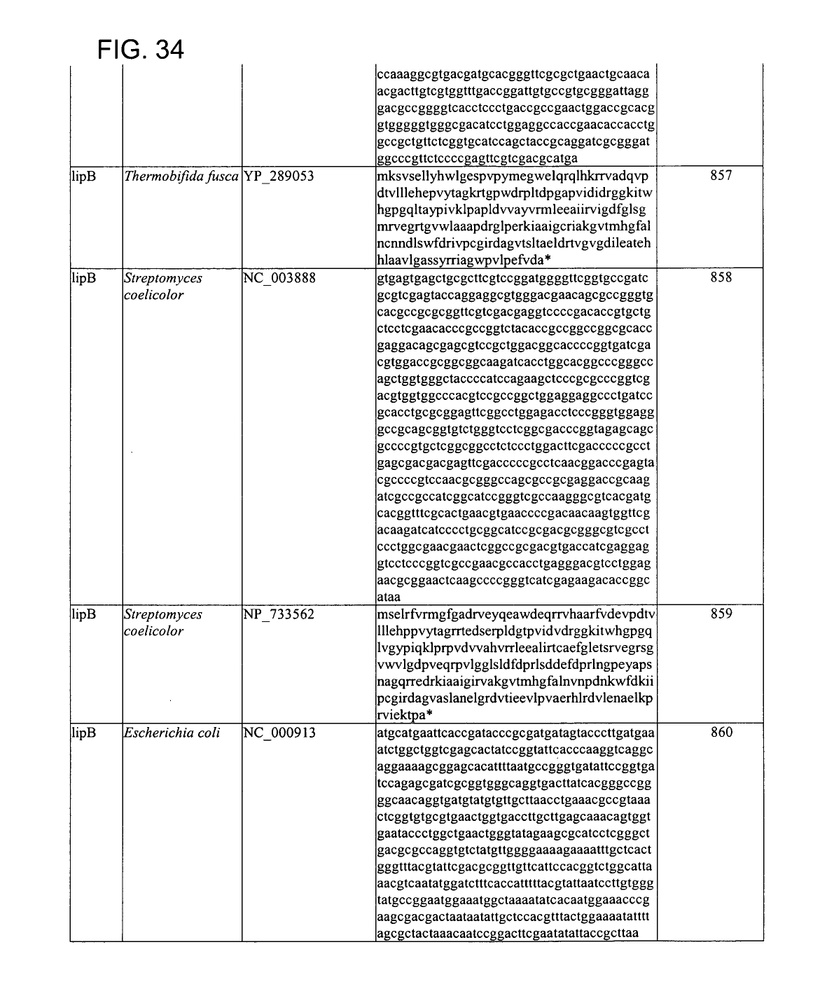

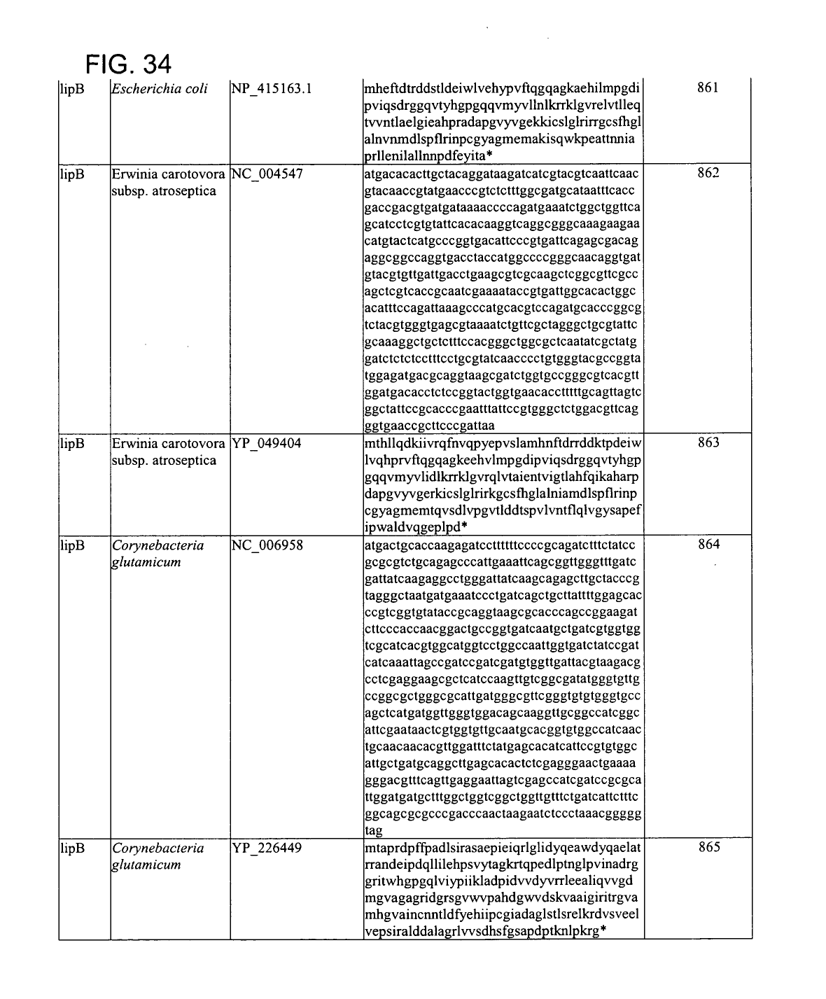

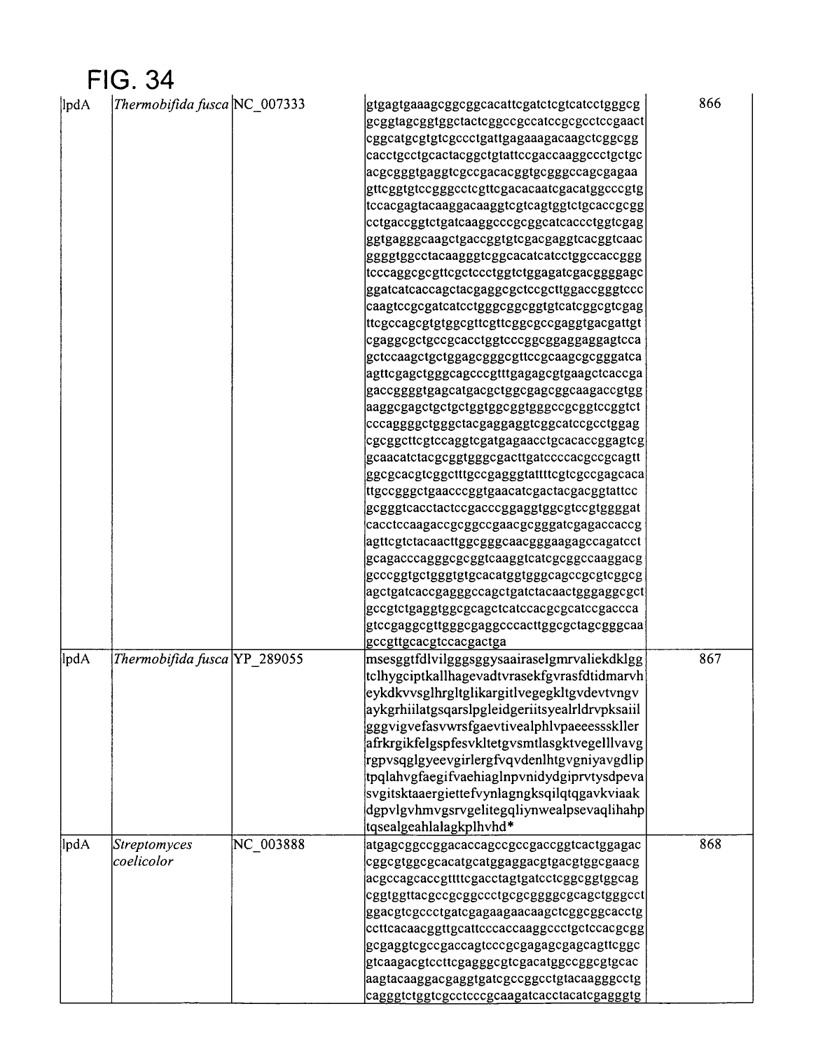

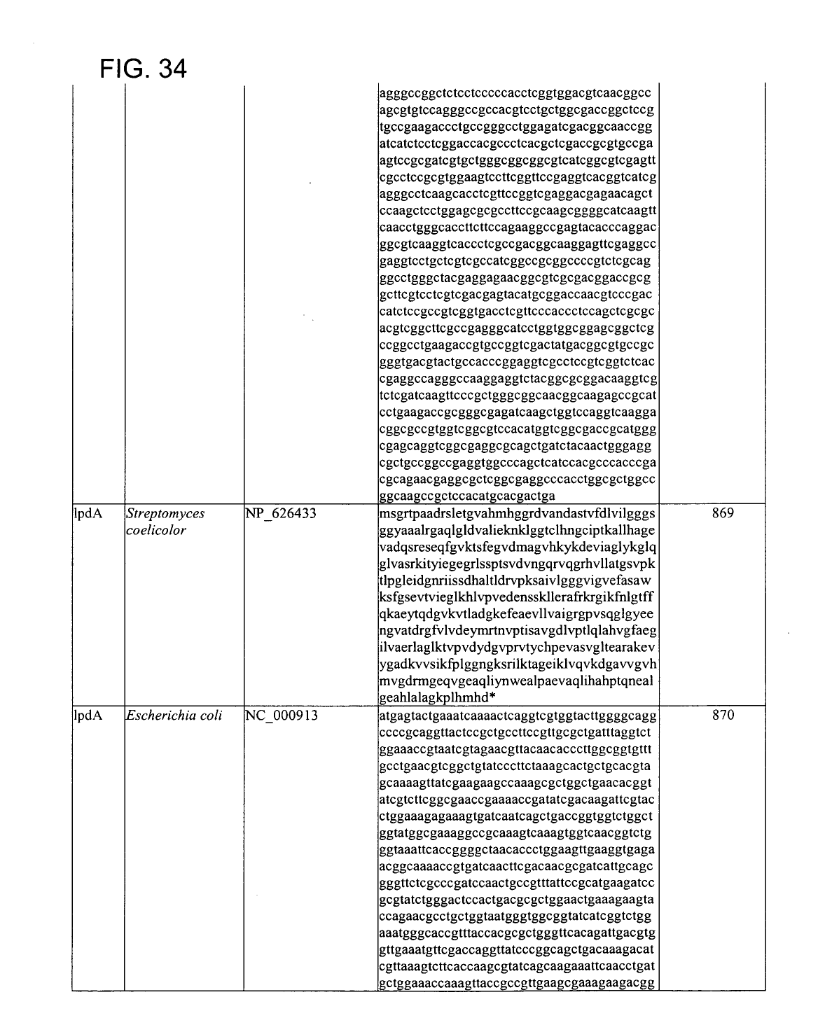

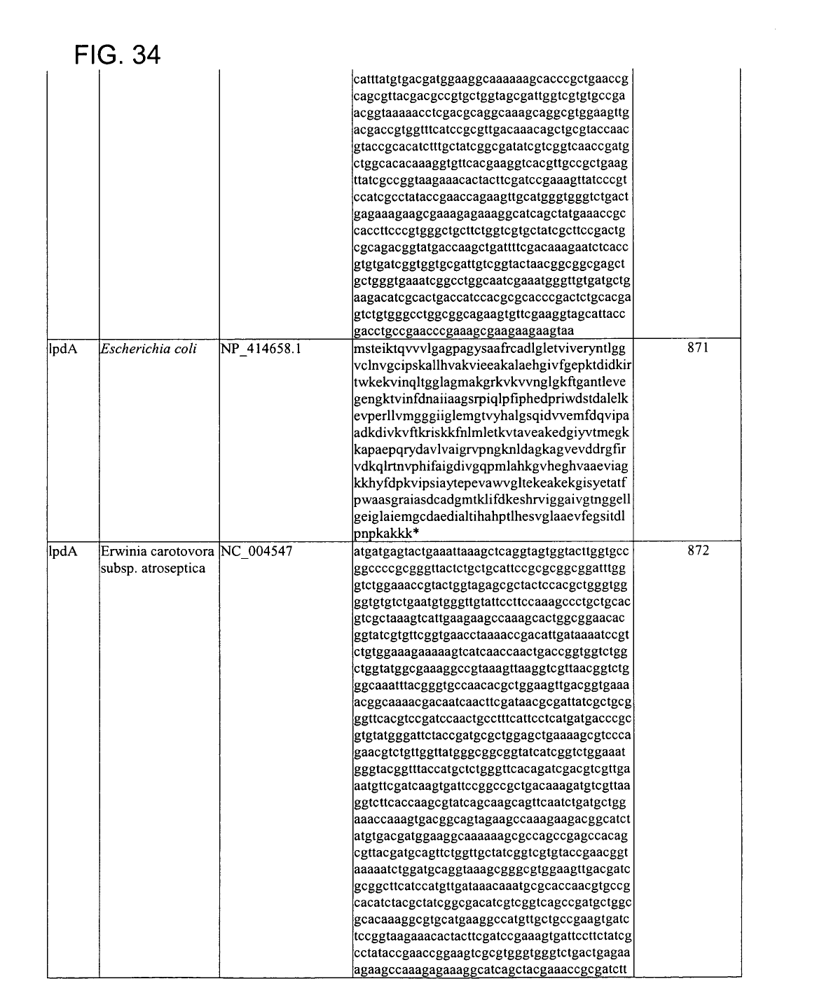

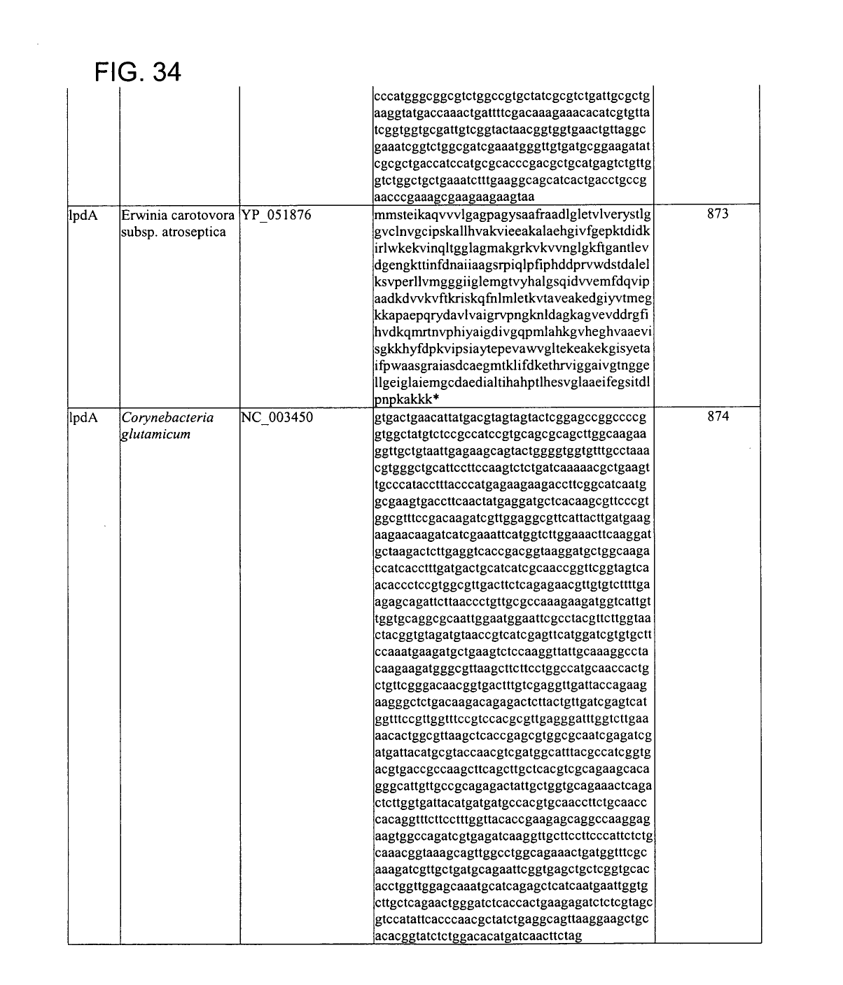

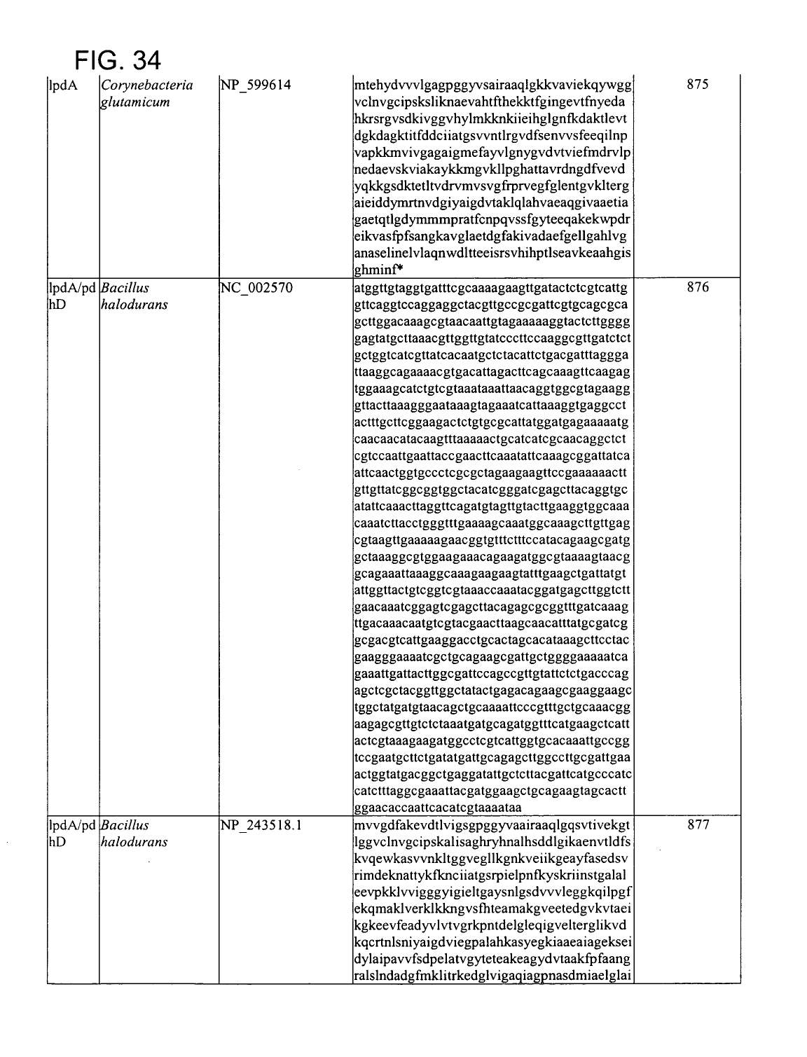

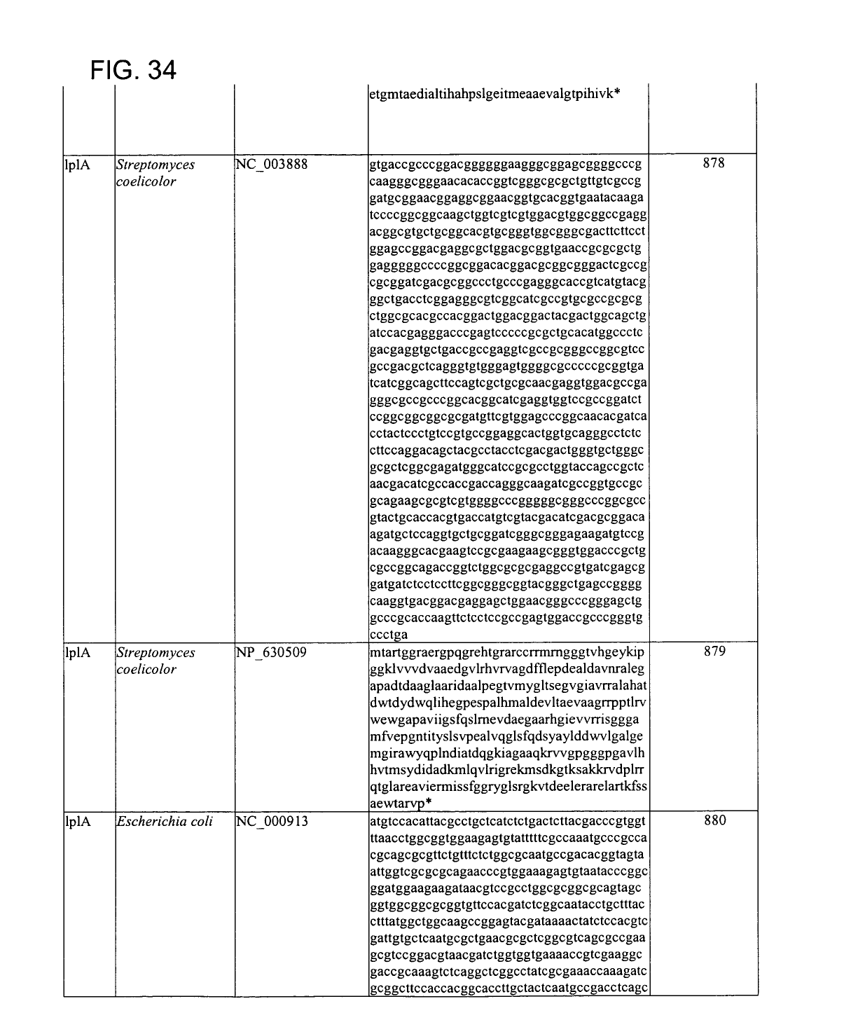

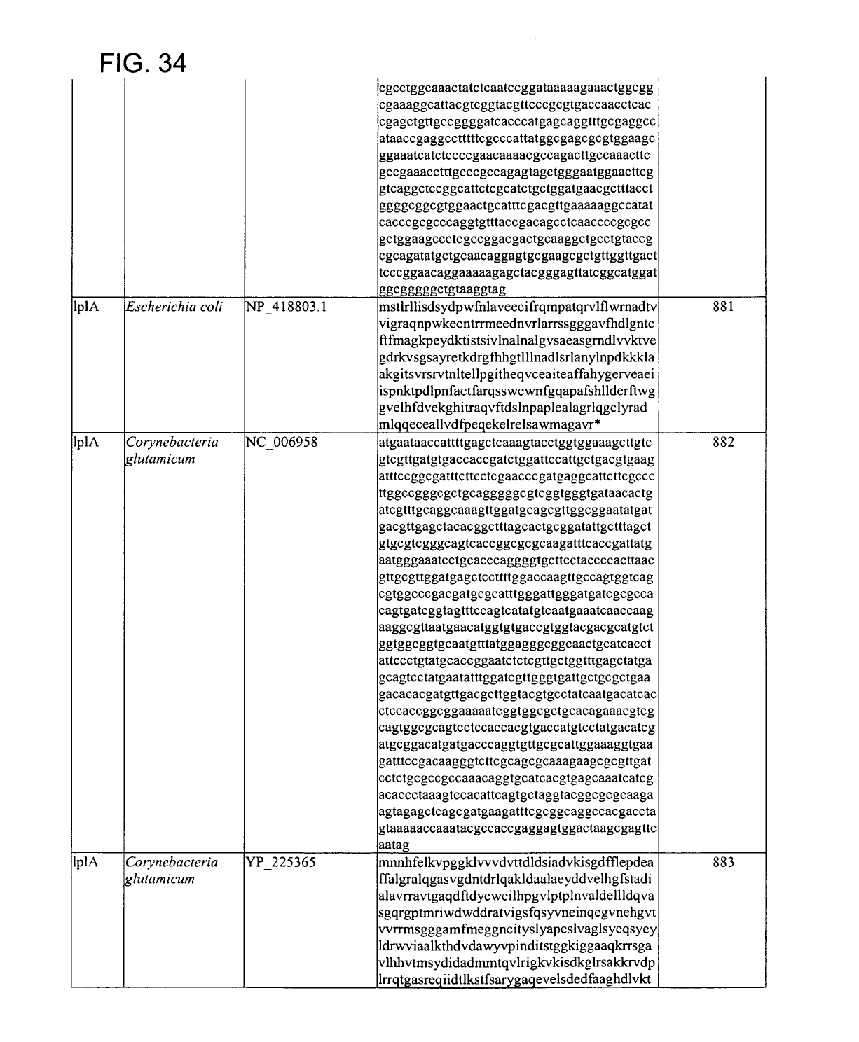

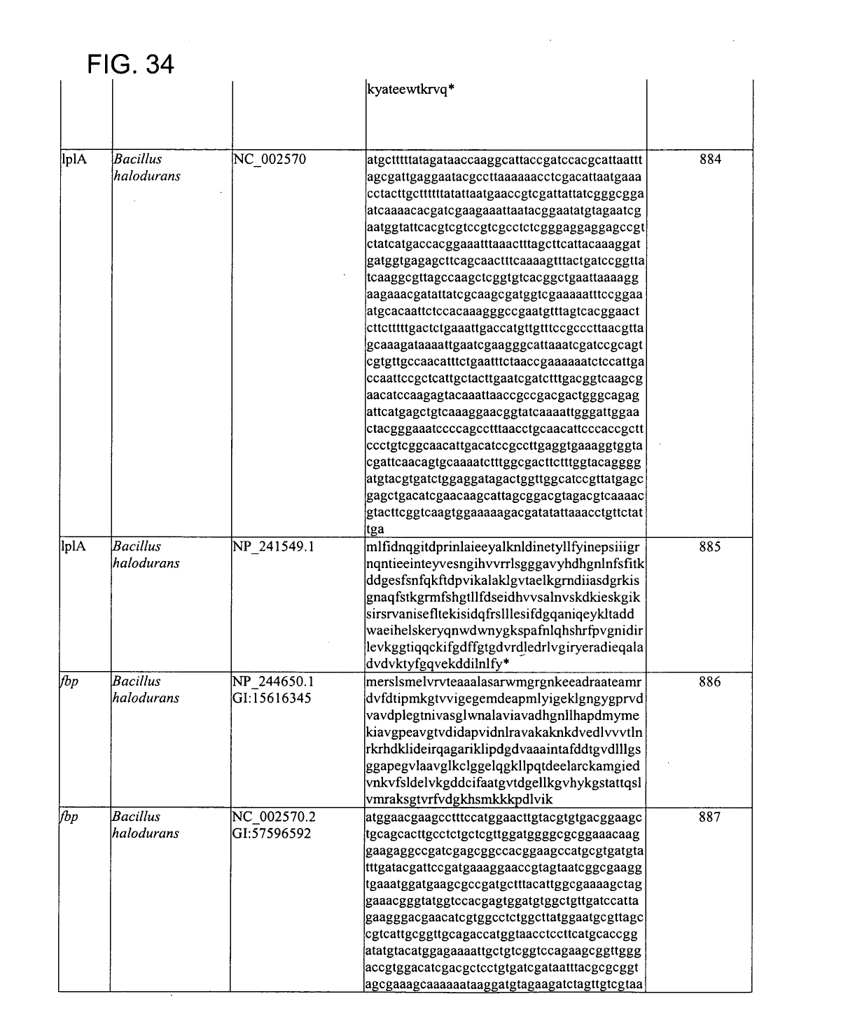

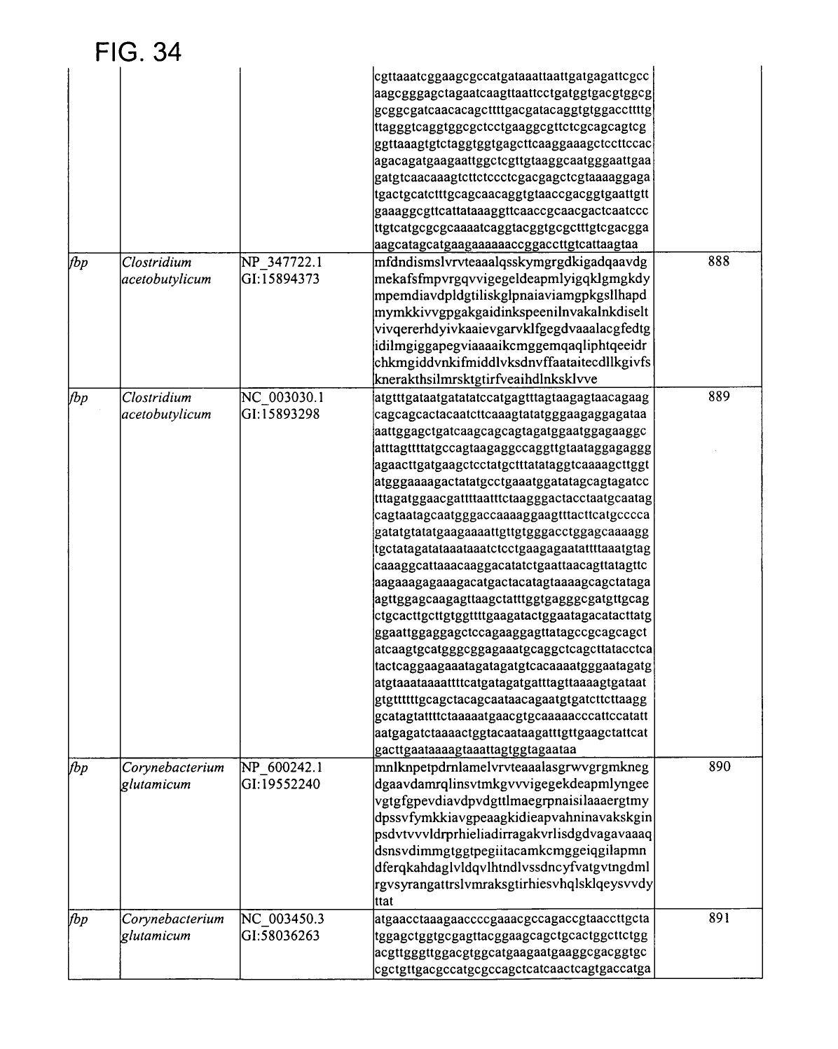

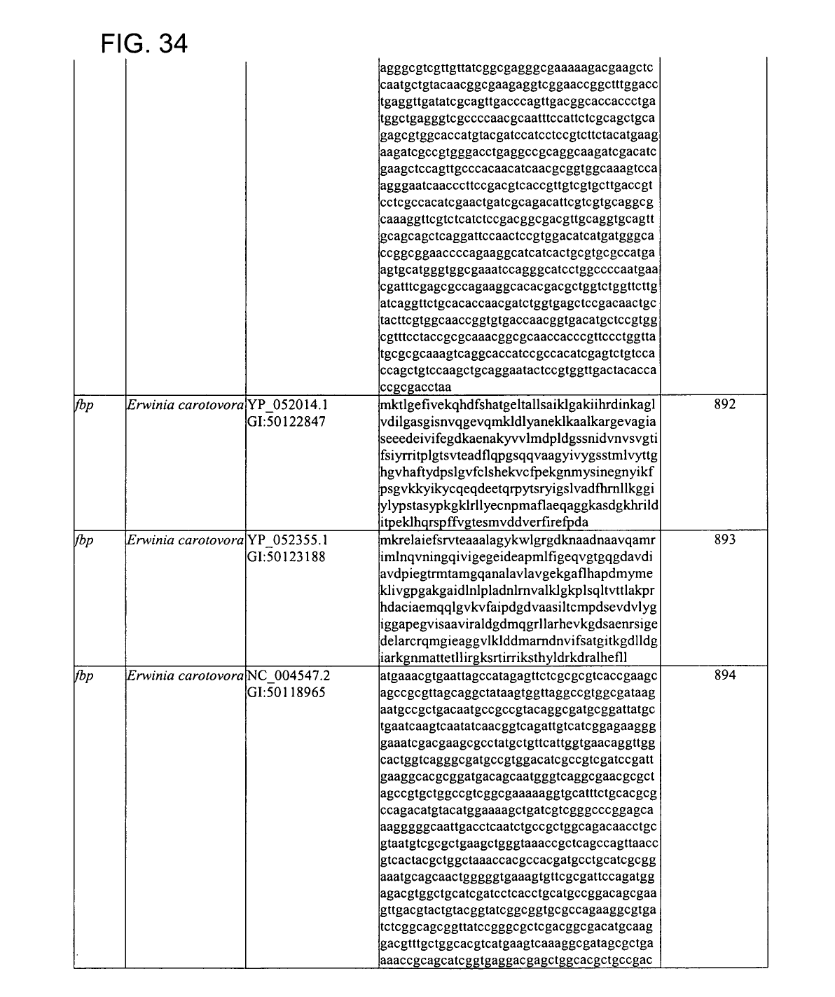

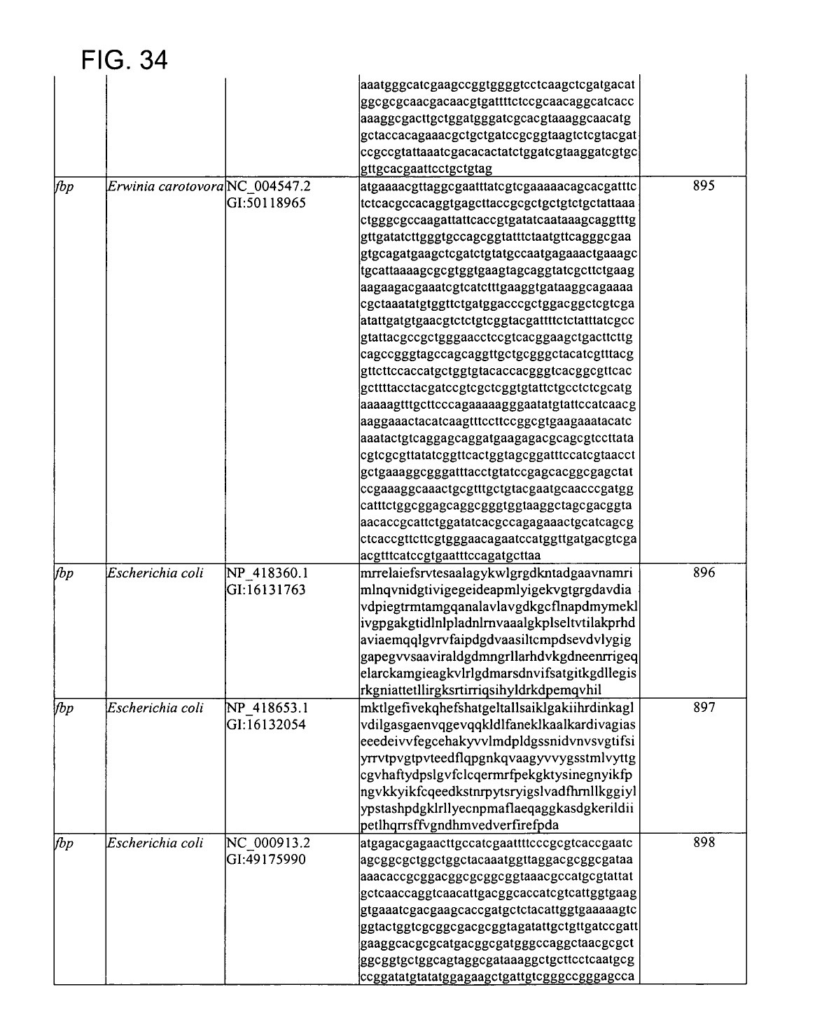

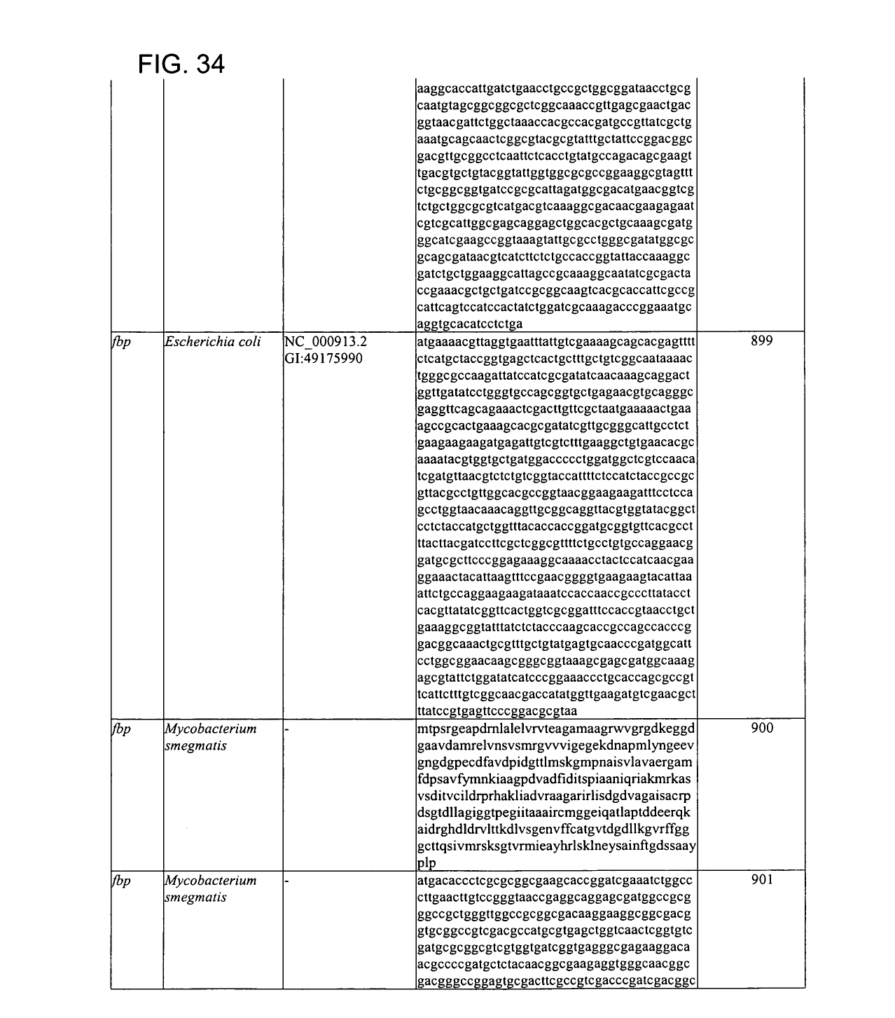

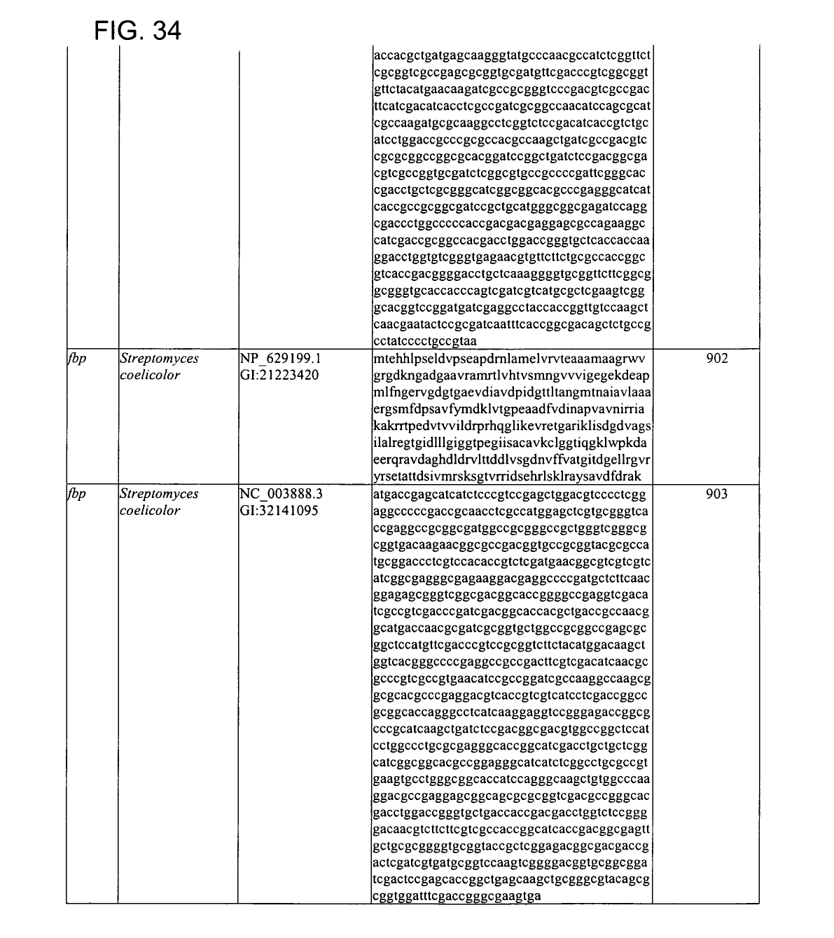

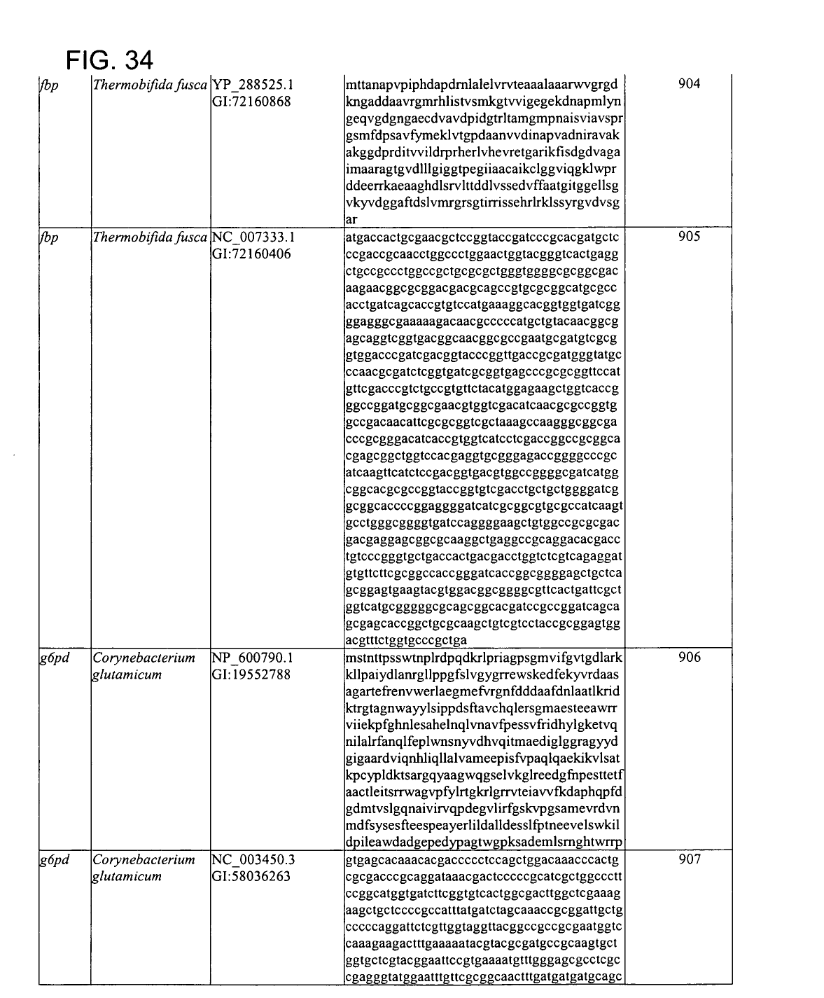

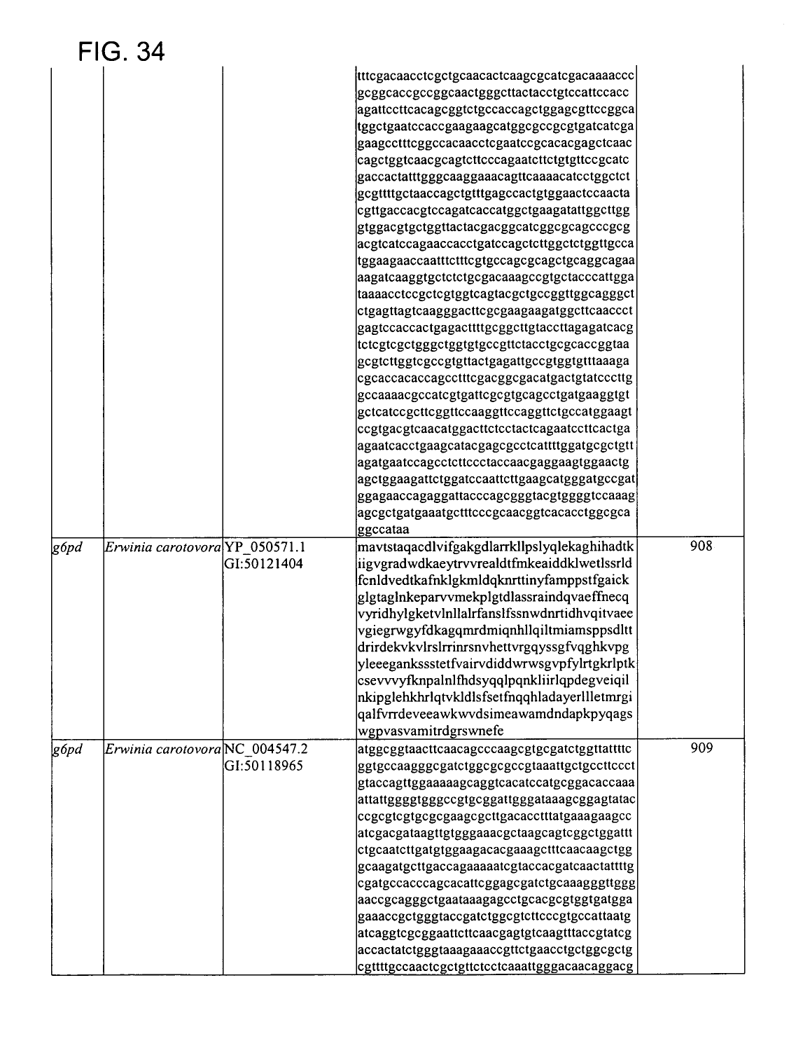

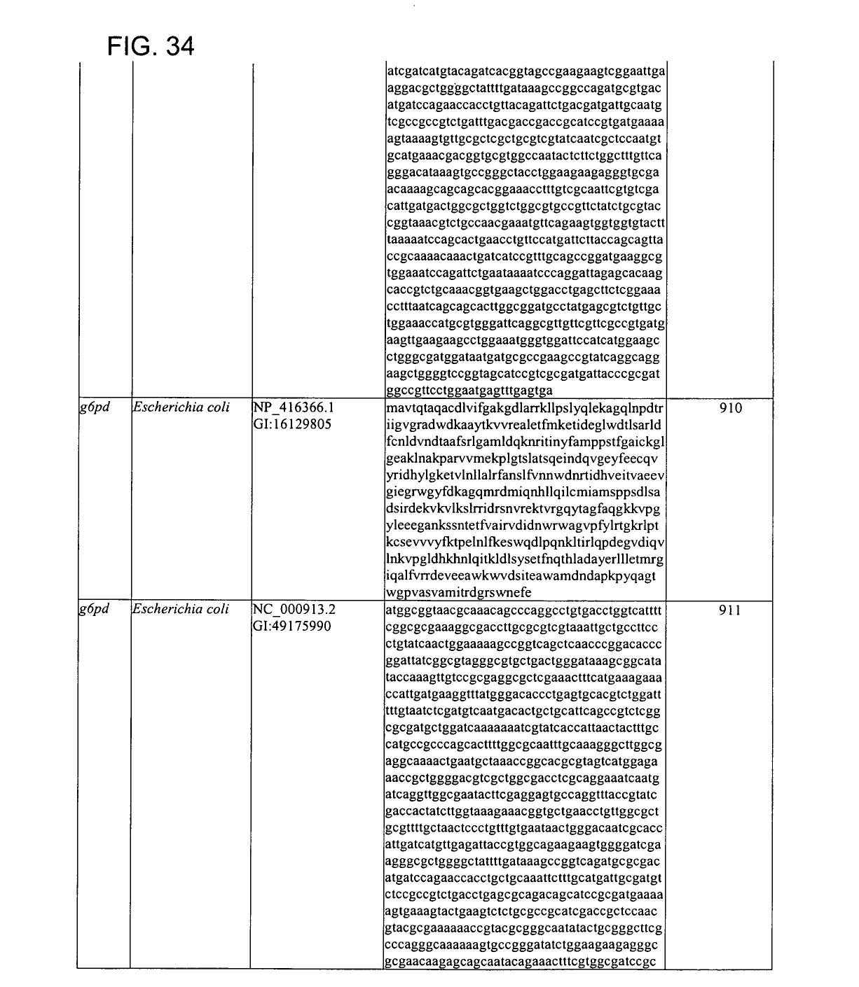

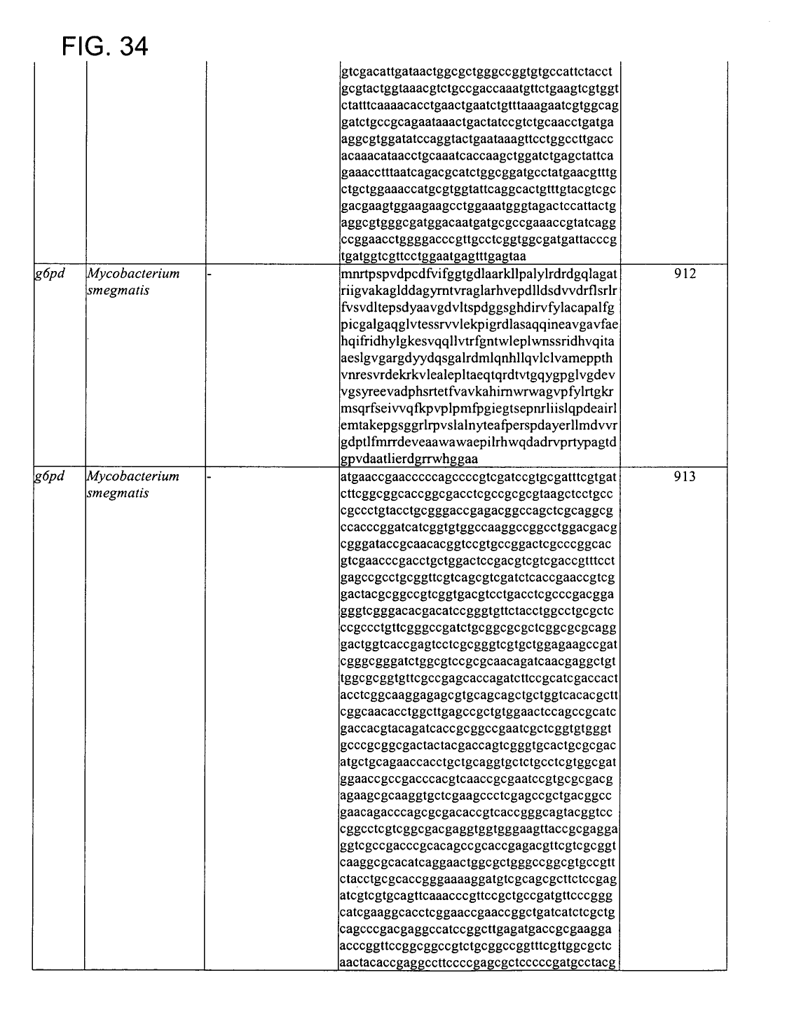

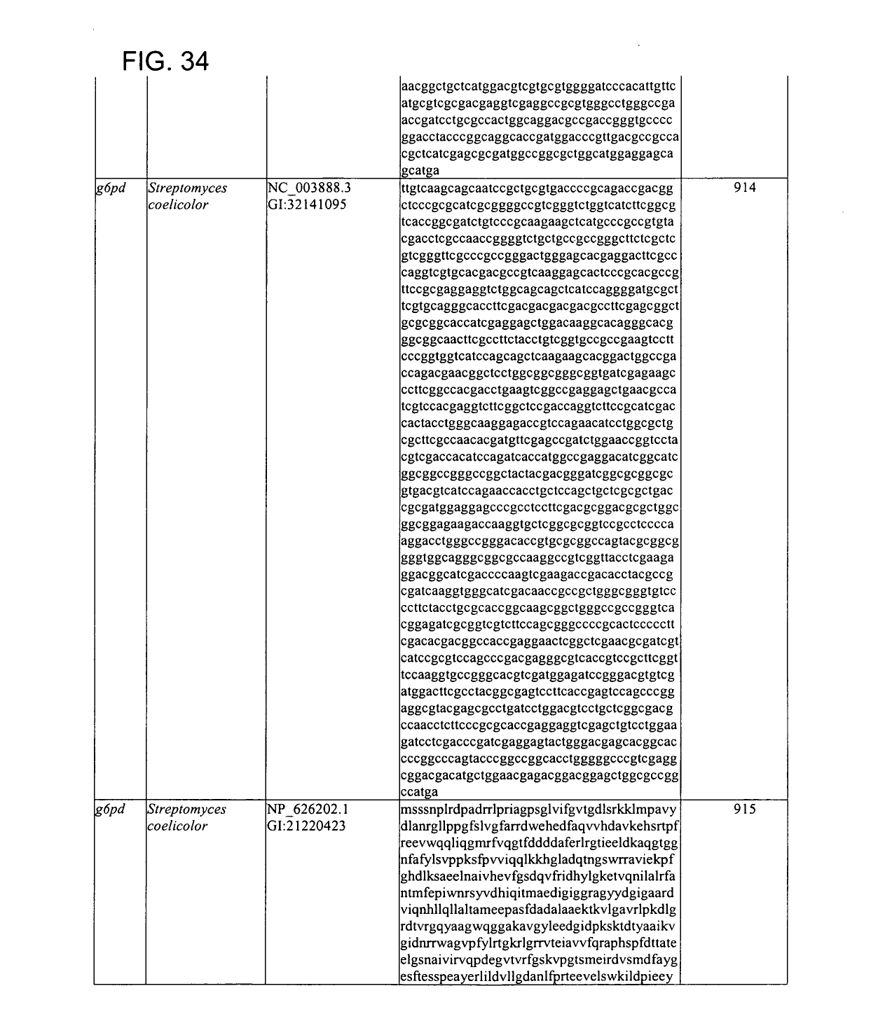

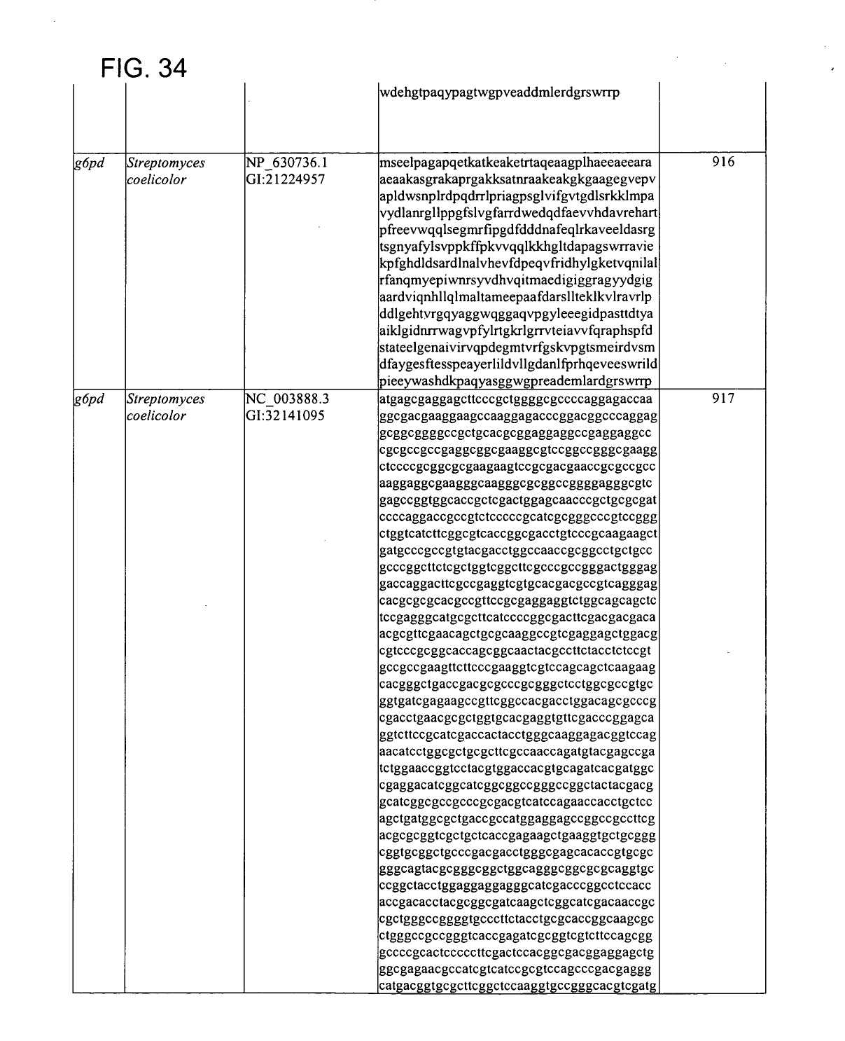

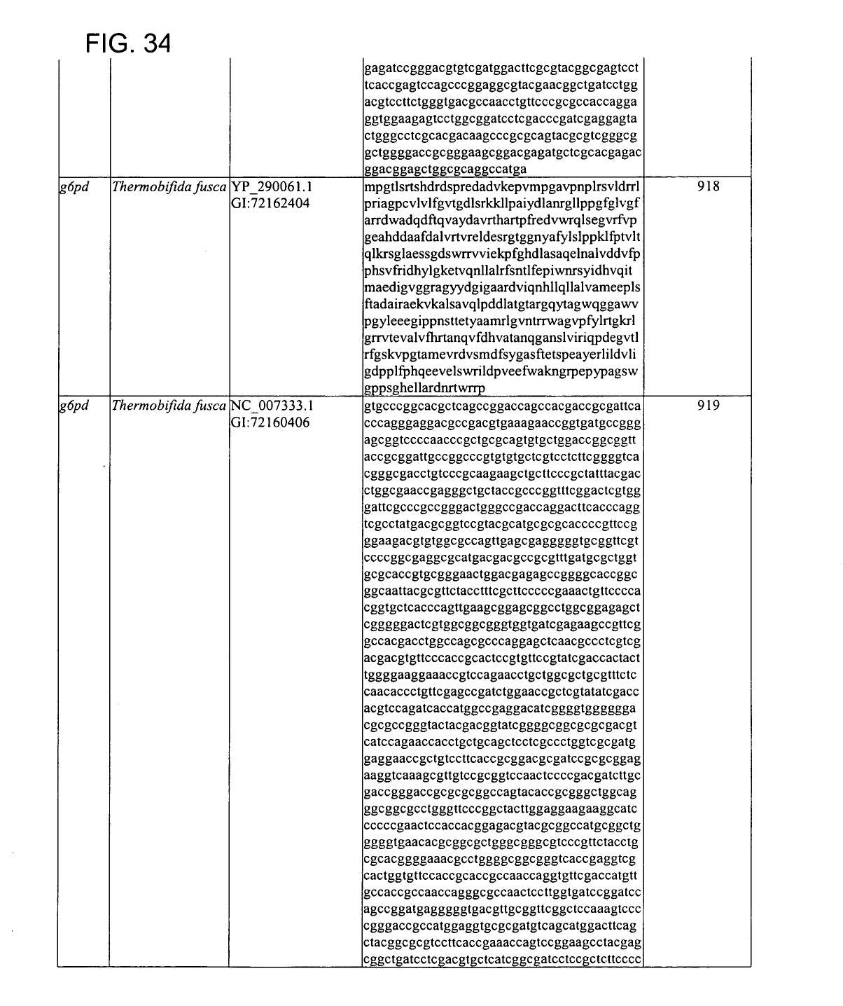

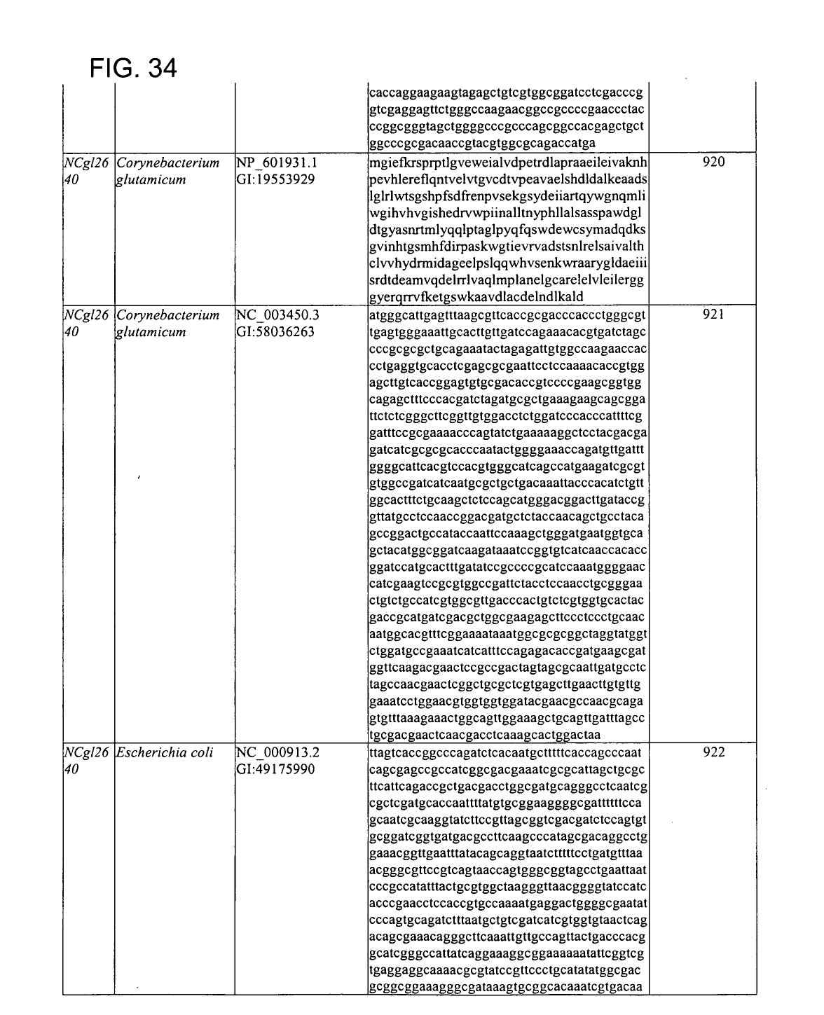

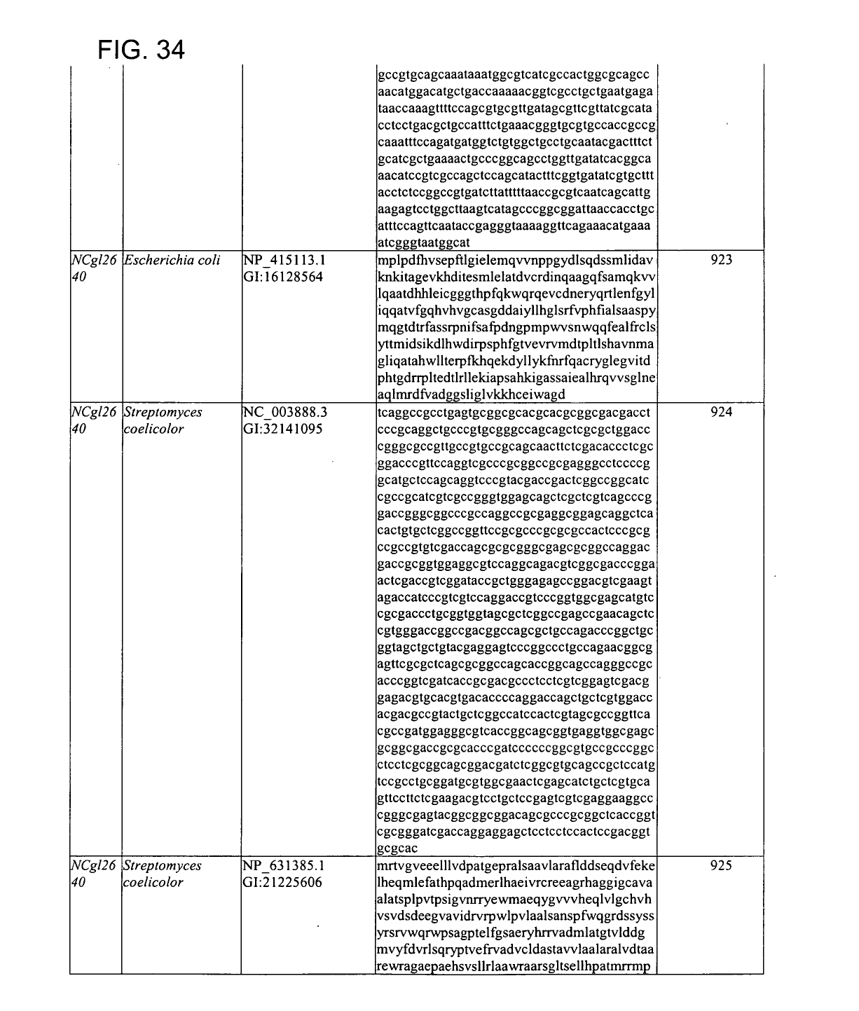

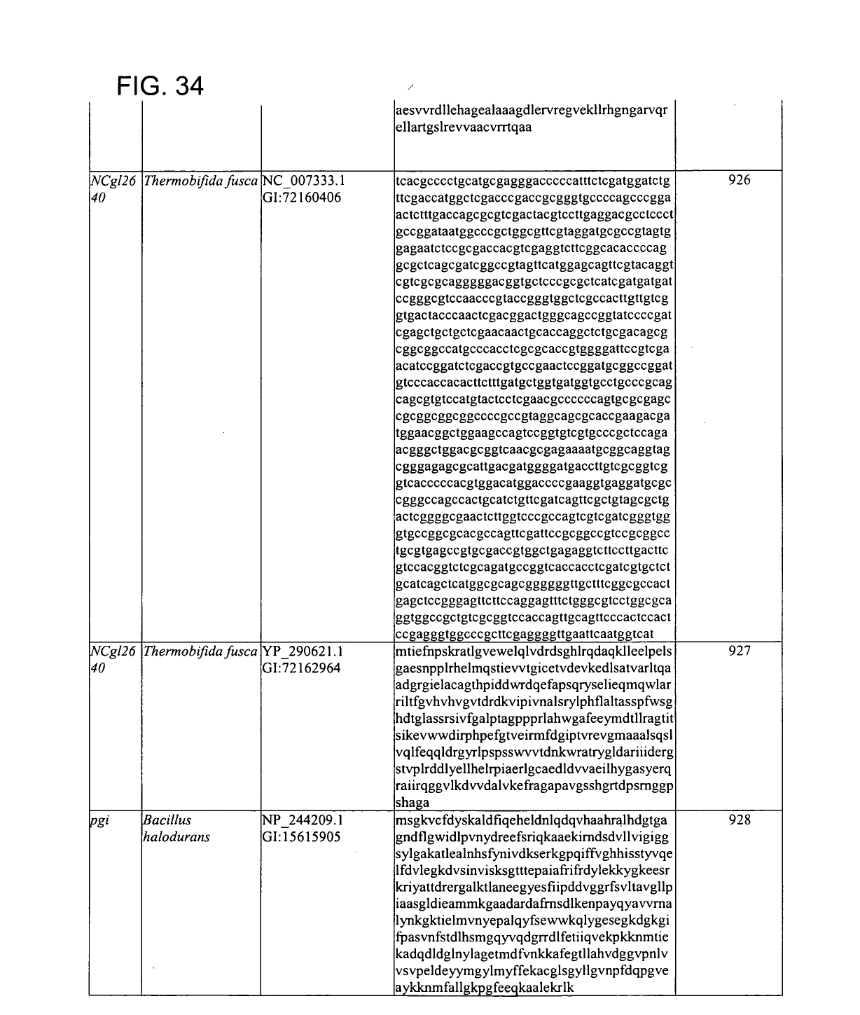

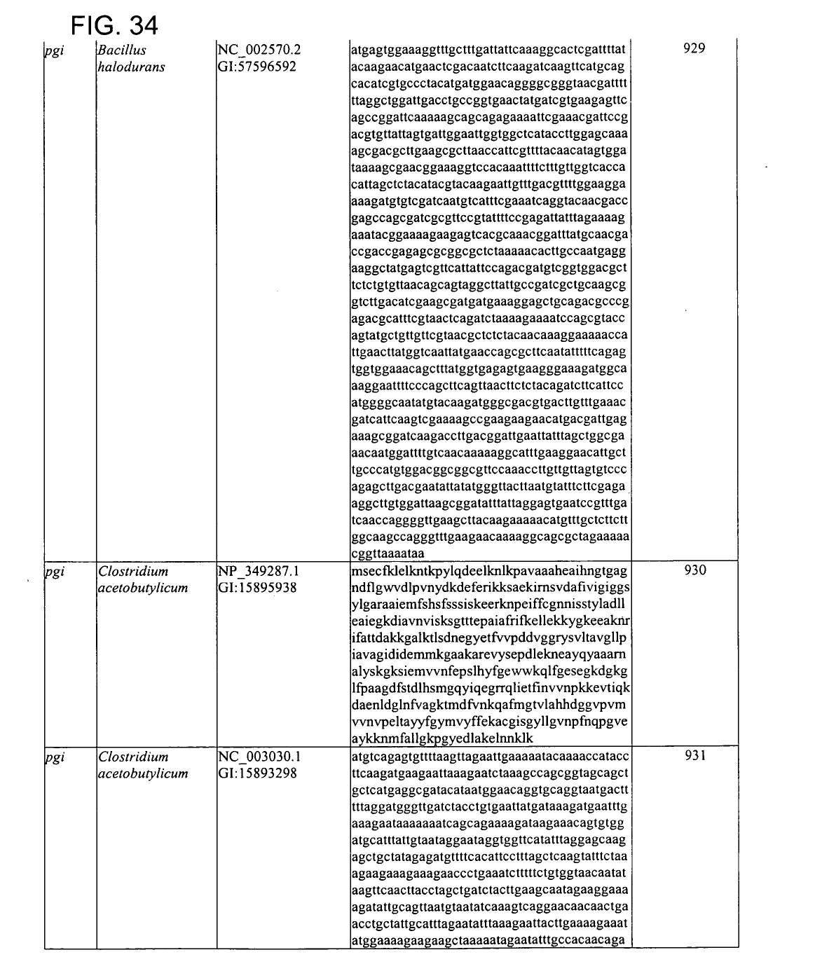

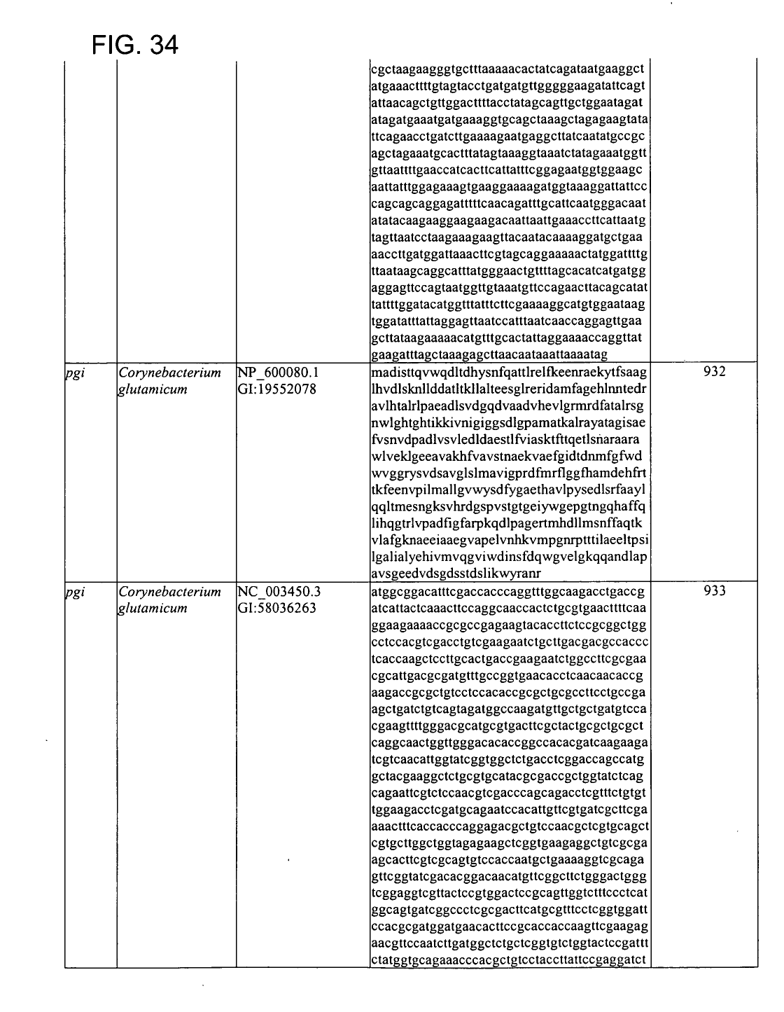

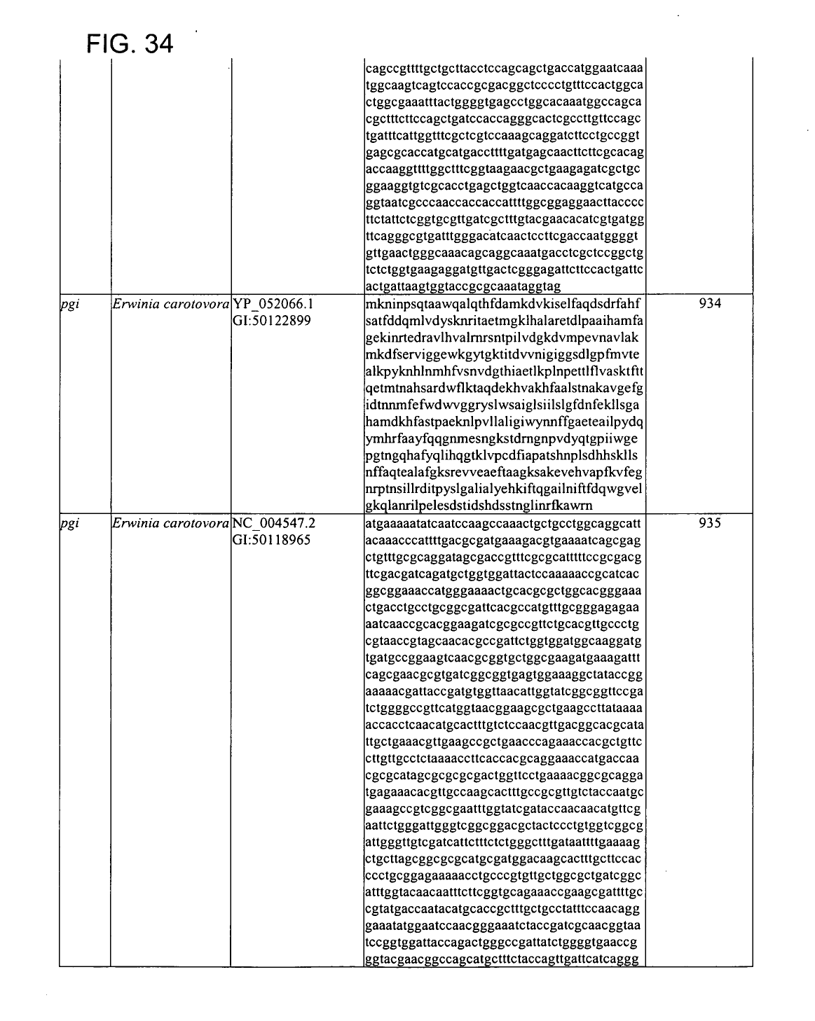

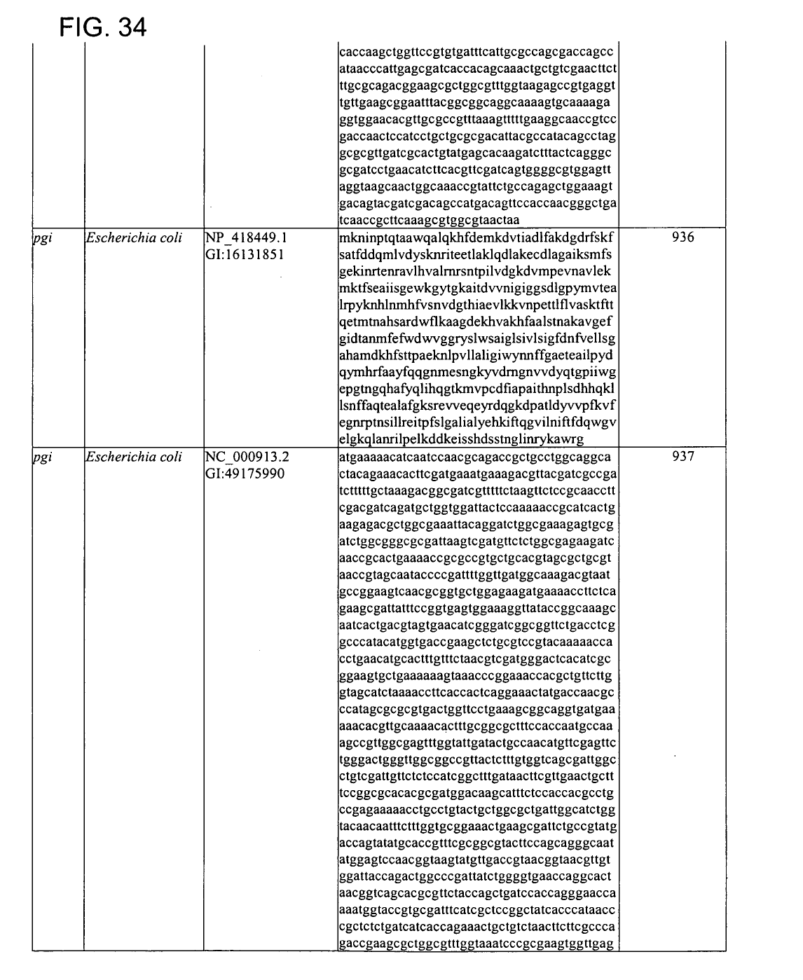

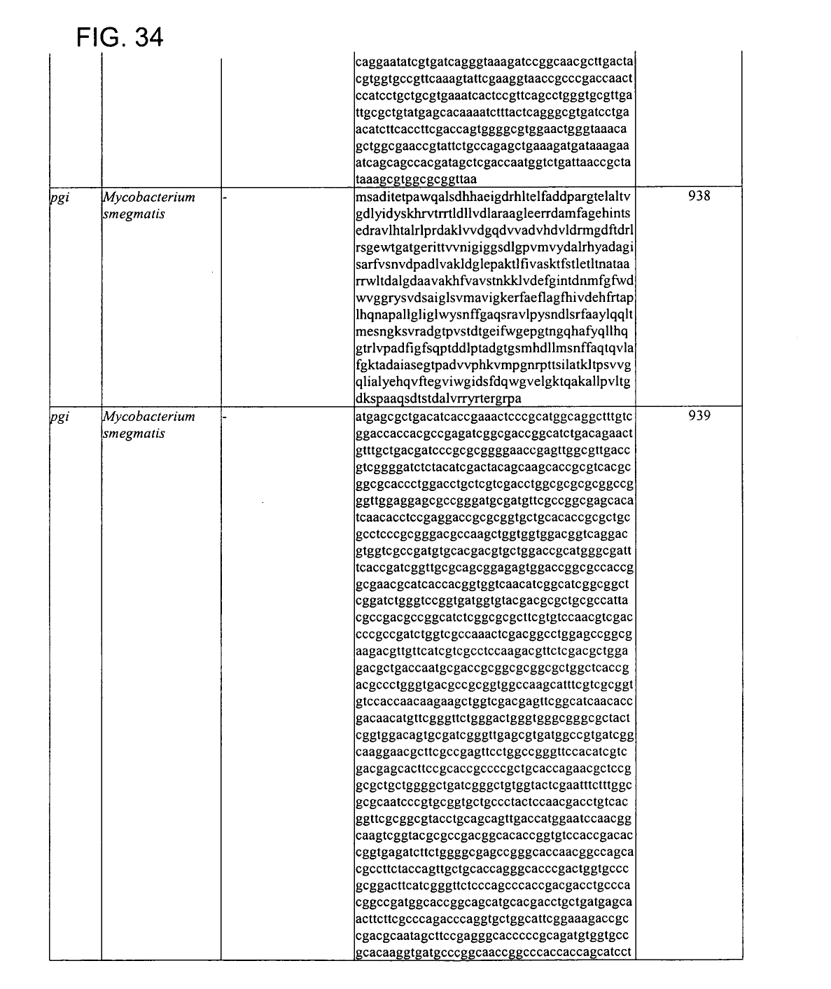

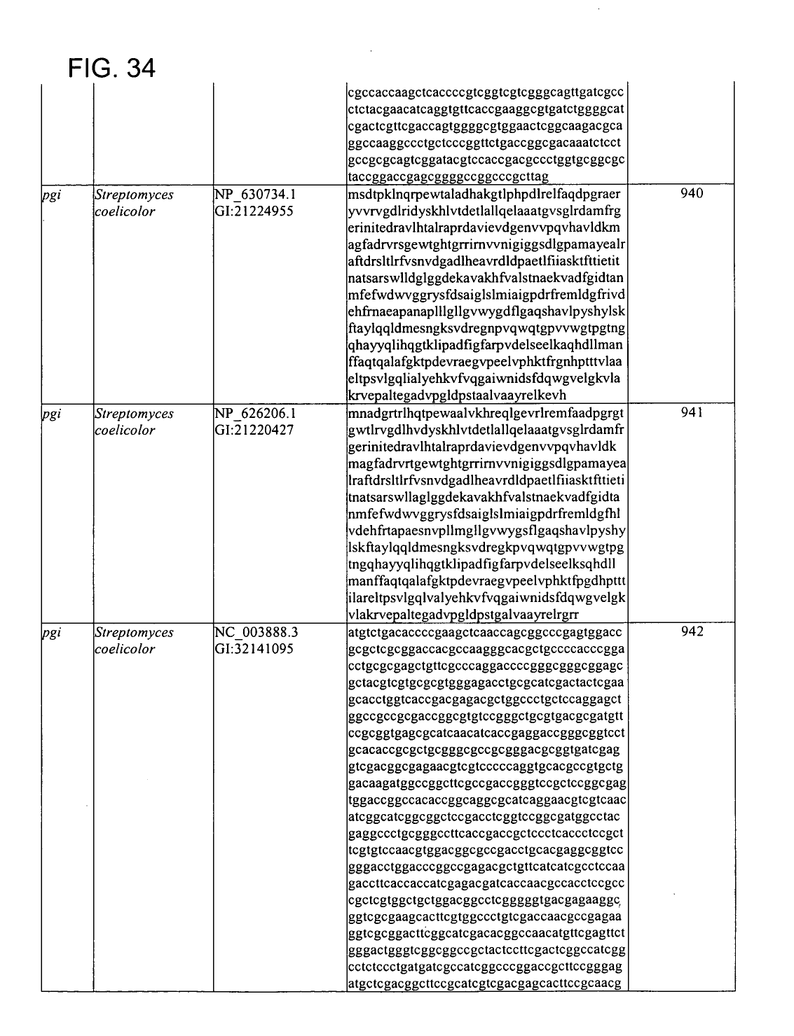

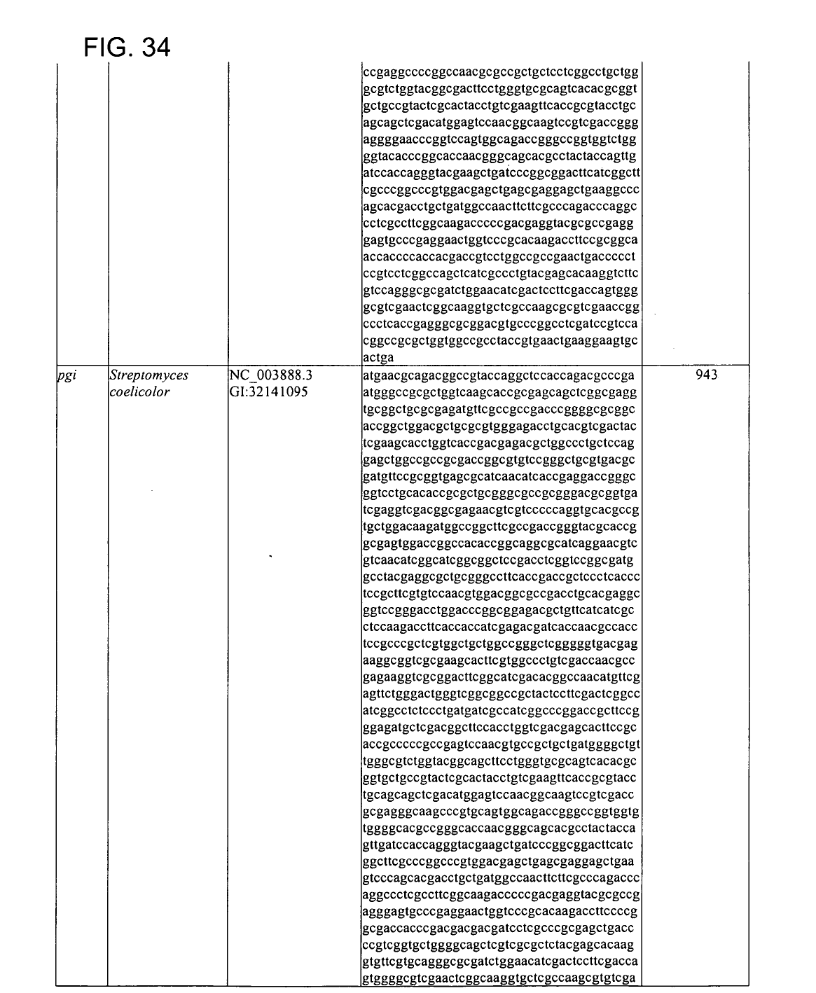

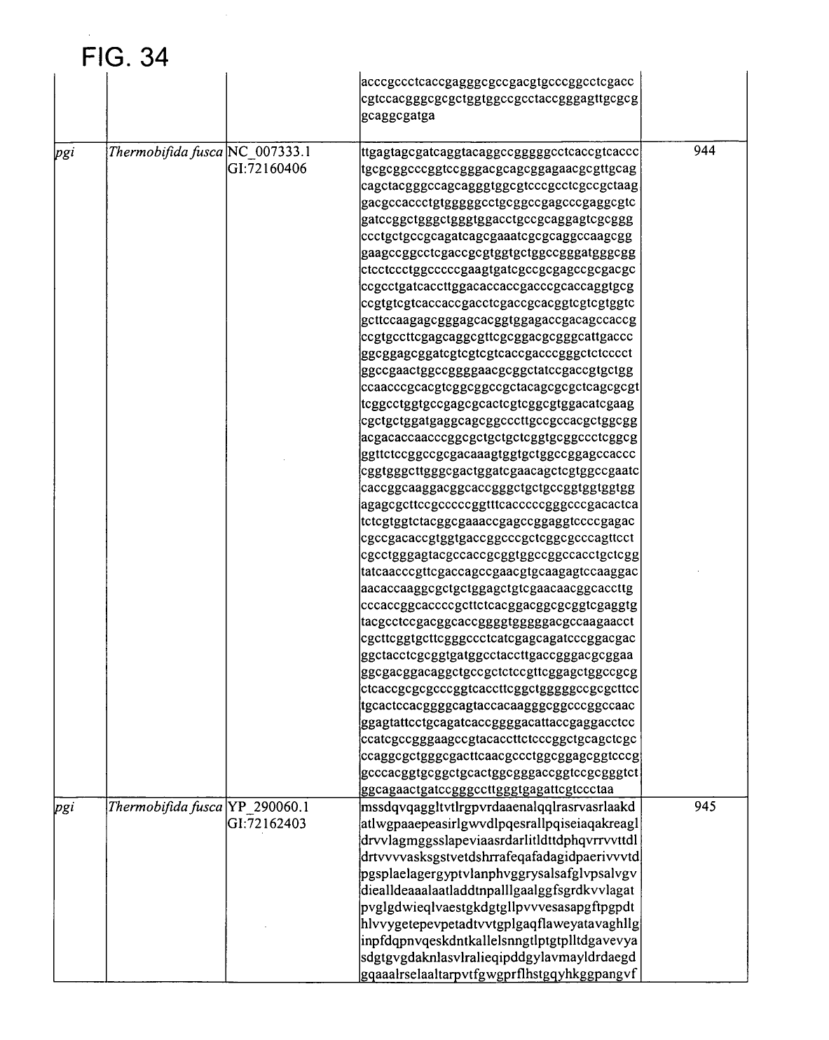

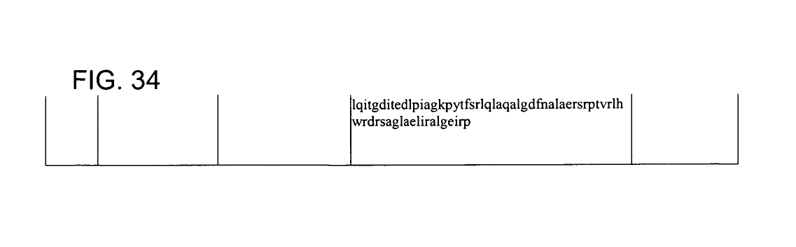

FIG. 32 shows the metabolite levels of strain MA-1668 and its parent strains. The scale for lysine and homoserine is on the left y-axis; the scale for methionine and O-acetylhomoserine is on the right y-axis. Strain MA-1668 was generated by transformation of MA-0993 with plasmid MB4287. Manipulation with MB4287 results in deletion of the mcbR locus and replacement with C. glutamicum metA(K233A)-metB. Strain MA-1668 produces approximately 2 g/L methionine, with decreased levels of lysine and homoserine relative to its progenitor strains. Strain MA-1668 is still amenable to further rounds of molecular manipulation.

Table 22 lists the strains used in these studies. The ‘::’ nomenclature indicates that the expression construct following the ‘::’ is integrated at the named locus prior to the ‘::’. EthR6 and EthR10 represent independently isolated ethionine resistant mutants. The Mcf3 mutation confers the ability to enable a Salmonella metE mutant to grow (see example 19). The Mms13 mutation confers methionine methylsulfonium chloride resistance (see example 15).

TABLE 22
Strains used in studies

| Name | Strain Genotype |
|---|---|
| MA-0002 | is ATCC 13032 |
| MA-0003 | is ATCC 13869 |
| MA-0008 | lacIq-trc-S. coelicolor lysC-asd(A191V) (episomal) |
| MA-0014 | lacIq-trc-M. smegmatis lysC-asd (episomal) |
| MA-0016 | lacIq-trc-M. smegmatis lysC (G345D)-asd (episomal) |
| MA-0019 | lacIq-trc-S. coelicolor lysC (S314I)-asd(A191V) (episomal) |
| MA-0022 | lacIq-trc-M. smegmatis lysC (T311I)-asd (episomal) |
| MA-0025 | lacIq-trc-M. smegmatis lysC (S301Y)-asd (episomal) |
| MA-0331 | Δhom-ΔthrB |
| MA-0333 | lacIq-trcRBS-M. smegmatis lysC (S301Y)-asd (episomal) |
| MA-0334 | lacIq-trcRBS-M. smegmatis lysC (T311I)-asd (episomal) |
| MA-0336 | lacIq-trcRBS-M. smegmatis lysC (G345D)-asd (episomal) |
| MA-0361 | gpd-M. smegmatis lysC (T311I)-asd (episomal) |
| MA-0362 | gpd-M. smegmatis lysC (G345D)-asd (episomal) |
| MA-0384 | Δhom-ΔthrB + rplM-S. coelicolor hom (G362E; G43S) (episomal) |
| MA-0386 | Δhom-ΔthrB + gpd-S. coelicolor hom (G362E; G43S) (episomal) |
| MA-0389 | Δhom-ΔthrB + lacIq-trcRBS-S. coelicolor hom (G362E; G43S; K19N) (episomal) |
| MA-0422 | EthR6 |
| MA-0428 | Δhom-ΔthrB::gpd-S. coelicolor hom (G362E; G43S) |
| MA-0442 | Δhom-ΔthrB + gpd-S. coelicolor hom (G362E; G43S) + lacIq-trcRBS-C. glutamicum |
| | metA-RBS-C. glutamicum metY (episomal) |
| MA-0449 | Δhom-ΔthrB + gpd-S. coelicolor hom (G362E; G43S) + lacIq-trcRBS-C. glutamicum |
| | metY-RBS-C. glutamicum metA (episomal) |
| MA-0456 | Δhom-ΔthrB::wgpd-S. coelicolor hom (G362E; G43S) + gpd-T. fusca metY-RBS-T. fusca |
| | metA (episomal) |
| MA-0463 | Δhom-ΔthrB::gpd-M. smegmatis lysC (T311I)-asd |
| MA-0466 | Δhom-ΔthrB + lacIq-trcRBS-E. chrysanthemi ppc (episomal) |
| MA-0472 | Δhom-ΔthrB + gpd-S. coelicolor dapA (episomal) |
| MA-0477 | Δhom-ΔthrB + lacIq-trcRBS-S. coelicolor dapA (episomal) |
| MA-0481 | Δhom-ΔthrB + gpd-E. chrysanthemi dapA (episomal) |
| MA-0482 | Δhom-ΔthrB + lacIq-trcRBS-E. chrysanthemi dapA (episomal) |
| MA-0486 | Δhom-ΔthrB::gpd-M. smegmatis lysC (T311I)-asd + lacIq-trcRBS-E. chsrysanthemi |
| | ppc (episomal) |
| MA-0492 | Δhom-ΔthrB::gpd-M. smegmatis lysC (T311I)-asd + gpd-S. coelicolor dapA |
| | (episomal) |
| MA-0497 | Δhom-ΔthrB::gpd-M. smegmatis lysC (T311I)-asd + lacIq-trcRBS-S. coelicolor |
| | dapA (episomal) |
| MA-0501 | Δhom-ΔthrB::gpd-M. smegmatis lysC (T311I)-asd + gpd-E. chrysanthemi dapA |
| | (episomal) |
| MA-0502 | Δhom-ΔthrB::gpd-M. smegmatis lysC (T311I)-asd + lacIq-trcRBS-E. chrysanthemi |
| | dapA (episomal) |
| MA-0569 | ΔmcbR + Δhom-ΔthrB::gpd-S. coelicolor hom (G362E; G43S) |
| MA-0570 | Δhom-ΔthrB + gpd-S. coelicolor hom (G362E; G43S) + lacIq-trcRBS-T. fusca |
| | metY-RBS-T. fusca metA (episomal) |
| MA-0578 | Δhom-ΔthrB + gpd-S. coelicolor hom (G362E; G43S) + gpd-T. fusca metA |
| | (episomal) |
| MA-0579 | Δhom-ΔthrB + gpd-S. coelicolor hom (G362E; G43S) + lacIq-trcRBS-T. fusca |
| | metA (episomal) |
| MA-0600 | Δhom-ΔthrB + gpd-S. coelicolor hom (G362E; G43S) + Mcf3 |
| MA-0622 | ΔmcbR + EthR6 |
| MA-0641 | ΔmcbR + EthR6 + gpd-C. glutamicum metA-RBS-C. glutamicum metY (episomal) |
| MA-0699 | mcbR + EthR6 + Δhom-ΔthrB::gpd-S. coelicolor hom (G362E) |
| MA-0721 | ΔmcbR + EthR6 + lacIq-trcRBS-C. glutamicum metA (K233A)-RBS-C. glutamicum |
| | metY (episomal) |
| MA-0725 | ΔmcbR + EthR6 + lacIq-trcRBS-C. glutamicum metA-RBS-C. glutamicum metY |
| | (D231A) (episomal) |
| MA-0727 | ΔmcbR + EthR6 + lacIq-trcRBS-C. glutamicum metA-RBS-C. glutamicum metY |
| | (G232A) (episomal) |
| MA-0933 | ΔthrB + ΔmcbR + EthR6 |
| MA-0993 | Δhom-ΔthrB::gpd-S. coelicolor hom (G362E; G43S) + Mcf3 + EthR10 |
| MA-1162 | ΔthrB + ΔmcbR + EthR6 + lacIq-trcRBS-M. smegmatis lysC (T311I)-asd (episomal) |
| MA-1351 | ΔthrB + ΔmcbR + EthR6 + lacIq-trcRBS-T. fusca metA (episomal) |
| MA-1378 | thrB + ΔmcbR + EthR6 + Mms13 + lacIq-trcRBS-M. smegmatis lysC (T311I)-asd |
| MA-1421 | Δhom-ΔthrB::gpd S. coelicolor hom (G362E; G43S) + ΔmcbR + Mcf3 + EthR10 |
| MA-1514 | ΔthrB + ΔmcbR + EthR6 + Mms13 |
| MA-1559 | ΔthrB + ΔmcbR + EthR6 + Mms13 + lacIq-trcRBS-T. fusca metA (episomal) |
| MA-1667 | ΔthrB + EthR6 + ΔmcbR::lacIq-trcRBS-T. fusca metA (episomal) |
| MA-1668 | Δhom-ΔthrB::gpd-S. coelicolor hom (G362E; G43S) + ΔmcbR::lacIq-trcRBS- |
| | C. glutamicum metA(K233A)-RBS-C. glutamicum metB + Mcf3 + EthR10 |
| MA-1688 | ΔmcbR + Δhom-ΔthrB::gpd-S. coelicolor hom (G362E; G43S) + lacIq-trcRBS-C. glutamicum |
| | metA-RBS-C. glutamicum metY-RBS-C. glutamicum metH |
| | (episomal) |
| MA-1743 | ΔhrB + ΔmcbR::lacIq-trcRBS-T. fusca metA + EthR6 + lacIq-trcRBS-C. glutamicum |
| | metA-RBS-C. glutamicum metY-RBS-C. glutamicum metH |
| | (episomal) |
| MA-1790 | Δhom-ΔthrB::gpd-S. coelicolor hom |
| | (G362E; G43S) + ΔmcbR + Mcf3 + EthR10 + lacIq-trcRBS-C. glutamicum metA- |
| | RBS-C. glutamicum-metY-RBS-C. glutamicum-metH (episomal) |
| MA-1906 | ΔmcbR + EthR6 + Δpck::gpd-M. smegmatis lysC (T311I)-asd |
| MA-1907 | ΔmcbR + EthR6 + Δpck::gpd-M. smegmatis lysC (T311I)-asd + ΔthrB |
| MA-2025 | ΔmcbR + EthR6 + Δpck::gpd-M. smegmatis lysC (T311I)-asd + ΔthrB + lacIq- |
| | trcRBS-C. glutamicum metA-RBS-C. glutamicum metY-RBS-C. glutamicum |
| | metH (episomal) |
| MA-2028 | ΔmcbR + EthR6 + Δpck::gpd-M. smegmatis lysC (T311I)-asd + lacIq-trcRBS-C. glutamicum |
| | metA-RBS-C. glutamicum metY-RBS-C. glutamicum metH |
| | (episomal) |

A number of embodiments of the invention have been described. Nevertheless, it will be understood that various modifications may be made without departing from the spirit and scope of the invention. Accordingly, other embodiments are within the scope of the following claims.

What is claimed is:

1. An Enterobacteriaceae or coryneform bacterium comprising at least one isolated nucleic acid molecule selected from the group consisting of:
(a) a nucleic acid molecule comprising a sequence encoding a bacterial sulfate ABC transporter ATP-binding polypeptide or a functional variant thereof;
(b) a nucleic acid molecule comprising a sequence encoding a bacterial sulfate transport system permease W polypeptide or a functional variant thereof;
(c) a nucleic acid molecule comprising a sequence encoding a bacterial sulfate, thiosulfate transport system permease T polypeptide or a functional variant thereof;
(d) a nucleic acid molecule comprising a sequence encoding a bacterial sulfate adenylyltransferase subunit 1 polypeptide or a functional variant thereof;
(e) a nucleic acid molecule comprising a sequence encoding a bacterial sulfate adenylyltransferase subunit 2 polypeptide or a functional variant thereof;
(f) a nucleic acid molecule comprising a sequence encoding a bacterial adenylylsulfate kinase polypeptide or a functional variant thereof;
(g) a nucleic acid molecule comprising a sequence encoding a bacterial phosphoadenosine phosphosulfate reductase polypeptide or a functional variant thereof;
(h) a nucleic acid molecule comprising a sequence encoding a bacterial sulfite reductase alpha subunit polypeptide or a functional variant thereof;
(i) a nucleic acid molecule comprising a sequence encoding a bacterial sulfite reductase hemopolypeptide beta-component polypeptide or a functional variant thereof;
(j) a nucleic acid molecule comprising a sequence encoding a bacterial sulfite reductase (NADPH), flavopolypeptide beta subunit polypeptide or a functional variant thereof;
(k) a nucleic acid molecule comprising a sequence encoding a bacterial adenylyl-sulphate reductase alpha subunit polypeptide or a functional variant thereof;
(l) a nucleic acid molecule comprising a sequence encoding a bacterial phosphoglycerate dehydrogenase polypeptide or a functional variant thereof;
(m) a nucleic acid molecule comprising a sequence encoding a bacterial phosphoserine transaminase polypeptide or a functional variant thereof;
(n) a nucleic acid molecule comprising a sequence encoding a bacterial phosphoserine phosphatase polypeptide or a functional variant thereof;
(o) a nucleic acid molecule comprising a sequence encoding a bacterial serine O-acetyltransferase polypeptide or a functional variant thereof;
(p) a nucleic acid molecule comprising a sequence encoding a bacterial cysteine synthase A polypeptide or a functional variant thereof;
(q) a nucleic acid molecule comprising a sequence encoding a bacterial cysteine synthase B polypeptide or a functional variant thereof;
(r) a nucleic acid molecule comprising a sequence encoding a bacterial ABC-type vitamin B12 transporter permease component polypeptide or a functional variant thereof;
(s) a nucleic acid molecule comprising a sequence encoding a bacterial ABC-type vitamin B12 transporter ATPase component polypeptide or a functional variant thereof;
(t) a nucleic acid molecule comprising a sequence encoding a bacterial ABC-type cobalamin/Fe³⁺-siderophore transport system polypeptide or a functional variant thereof;
(u) a nucleic acid molecule comprising a sequence encoding a bacterial adenosyltransferase polypeptide or a functional variant thereof;
(v) a nucleic acid molecule comprising a sequence encoding a bacterial GTP cyclohydrolase I polypeptide or a functional variant thereof;
(w) a nucleic acid molecule comprising a sequence encoding a bacterial phoA, psiA, or psiF gene product polypeptide or a functional variant thereof;
(x) a nucleic acid molecule comprising a sequence encoding a bacterial dihydroneopterin aldolase polypeptide or a functional variant thereof;
(y) a nucleic acid molecule comprising a sequence encoding a bacterial 7,8-dihydro-6-hydroxymethylpterin-pyrophosphokinase polypeptide or a functional variant thereof;
(z) a nucleic acid molecule comprising a sequence encoding a bacterial dihydropteroate synthase polypeptide or a functional variant thereof;
(aa) a nucleic acid molecule comprising a sequence encoding a bacterial dihydrofolate synthetase polypeptide or a functional variant thereof;
(ab) a nucleic acid molecule comprising a sequence encoding a bacterial dihydrofolate reductase polypeptide or a functional variant thereof;
(ac) a nucleic acid molecule comprising a sequence encoding a bacterial folylpolyglutamate synthetase polypeptide or a functional variant thereof;
(ad) a nucleic acid molecule comprising a sequence encoding a putative bacterial methionine (APC transporter superfamily) permease (YjeH) polypeptide or a functional variant thereof;
(ae) a nucleic acid molecule comprising a sequence encoding a bacterial transcriptional activator of MetE/H polypeptide or a functional variant thereof;
(af) a nucleic acid molecule comprising a sequence encoding a bacterial 6-phosphogluconate dehydrogenase polypeptide or a functional variant thereof;
(ag) a nucleic acid molecule comprising a sequence encoding a bacterial S-methylmethionine homocysteine methyltransferase polypeptide or a functional variant thereof;
(ah) a nucleic acid molecule comprising a sequence encoding a bacterial S-adenosylhomocysteine hydrolase polypeptide or a functional variant thereof;
(ai) a nucleic acid molecule comprising a sequence encoding a bacterial site-specific DNA methylase polypeptide or a functional variant thereof;
(aj) a nucleic acid molecule comprising a sequence encoding a bacterial methionine export sytem protein 1 polypeptide or a functional variant thereof;
(ak) a nucleic acid molecule comprising a sequence encoding a bacterial methionine export sytem protein 2 polypeptide or a functional variant thereof;
(al) a nucleic acid molecule comprising a sequence encoding a bacterial ABC transport system ATP-binding protein (MetN) polypeptide or a functional variant thereof;
(am) a nucleic acid molecule comprising a sequence encoding a bacterial ABC transport system permease protein (MetP) polypeptide or a functional variant thereof;
(an) a nucleic acid molecule comprising a sequence encoding a bacterial ABC transport system substrate-binding protein (MetQ) polypeptide or a functional variant thereof;
(ao) a nucleic acid molecule comprising a sequence encoding a bacterial aspartokinase polypeptide or a functional variant thereof;
(ap) a nucleic acid molecule comprising a sequence encoding a bacterial aspartate semialdehyde dehydrogenase or a functional variant thereof;
(aq) a nucleic acid molecule comprising a sequence encoding a bacterial homoserine dehydrogenase polypeptide or a functional variant thereof;
(ar) a nucleic acid molecule comprising a sequence encoding a bacterial O-homoserine acetyl transferase polypeptide or a functional variant thereof;
(as) a nucleic acid molecule comprising a sequence encoding a bacterial O-acetylhomoserine sulfhydrylase polypeptide or a functional variant thereof;
(at) a nucleic acid molecule comprising a sequence encoding a bacterial cobalamin-dependent methionine synthase polypeptide or a functional variant thereof;
(au) a nucleic acid molecule comprising a sequence encoding a bacterial cobalamin-independent methionine synthase polypeptide or a functional variant thereof;
(av) a nucleic acid molecule comprising a sequence encoding a bacterial homoserine kinase polypeptide or a functional variant thereof;
(aw) a nucleic acid molecule comprising a sequence encoding a bacterial methionine adenosyltransferase polypeptide or a functional variant thereof;
(ax) a nucleic acid molecule comprising a sequence encoding a bacterial O-succinylhomoserine (thio)-lyase polypeptide or a functional variant thereof;
(ay) a nucleic acid molecule comprising a sequence encoding a bacterial cystathionine beta-lyase polypeptide or a functional variant thereof;
(az) a nucleic acid molecule comprising a sequence encoding a bacterial 5,10-methylenetetrahydrofolate reductase polypeptide or a functional variant thereof;
(ba) a nucleic acid molecule comprising a sequence encoding a bacterial dihydrodipicolinate synthase polypeptide or a functional variant thereof;
(bb) a nucleic acid molecule comprising a sequence encoding a bacterial pyruvate carboxylase polypeptide or a functional variant thereof;
(bc) a nucleic acid molecule comprising a sequence encoding a bacterial glutamate dehydrogenase polypeptide or a functional variant thereof;
(bd) a nucleic acid molecule comprising a sequence encoding a bacterial diaminopimelate dehydrogenase polypeptide or a functional variant thereof;
(be) a nucleic acid molecule comprising a sequence encoding a bacterial methionine and cysteine biosynthesis repressor (McbR) polypeptide or a functional variant thereof;
(bf) a nucleic acid molecule comprising a sequence encoding a bacterial lysine exporter protein polypeptide or a functional variant thereof;
(bg) a nucleic acid molecule comprising a sequence encoding a bacterial phosphoenolpyruvate carboxykinase polypeptide or a functional variant thereof;
(bh) a nucleic acid molecule comprising a sequence encoding a bacterial phosphoenolpyruvate carboxylase polypeptide or a functional variant thereof;
(bi) a nucleic acid molecule comprising a sequence encoding a bacterial glycine dehydrogenase (decarboxylating) polypeptide or a functional variant thereof;
(bj) a nucleic acid molecule comprising a sequence encoding a bacterial H polypeptide (involved in the glycine cleavage system) or a functional variant thereof;
(bk) a nucleic acid molecule comprising a sequence encoding a bacterial aminomethyl transferase polypeptide or a functional variant thereof;
(bl) a nucleic acid molecule comprising a sequence encoding a bacterial dihydrolipoamide dehydrogenase polypeptide or a functional variant thereof;
(bm) a nucleic acid molecule comprising a sequence encoding a bacterial lipoate-protein ligase A polypeptide or a functional variant thereof;
(bn) a nucleic acid molecule comprising a sequence encoding a bacterial lipoic acid synthase polypeptide or a functional variant thereof;
(bo) a nucleic acid molecule comprising a sequence encoding a bacterial lipoyl-[acyl-carrier-protein]-protein-N-lipoyltransferase polypeptide or a functional variant thereof;
(bp) a nucleic acid molecule comprising a sequence encoding a bacterial fructose 1,6 bisphosphatase polypeptide or a functional variant thereof;
(bq) a nucleic acid molecule comprising a sequence encoding a bacterial glucose 6 phosphate dehydrogenase polypeptide or a functional variant thereof;
(br) a nucleic acid molecule comprising a sequence encoding a glucose-6-phosphate isomerase polypeptide or a functional variant thereof; and
(bs) a nucleic acid molecule comprising a sequence encoding a bacterial NCgl2640 polypeptide or a functional variant thereof

2. The bacterium of claim 1 wherein the bacterium comprises at least two of nucleic acid molecules (a)-(bs).

3. The bacterium of claim 1 wherein the bacterium comprises at least three of nucleic acid molecules (a)-(bs).

4. The bacterium of claim 1 wherein the bacterium comprises at least four of nucleic acid molecules (a)-(bs).

5. The bacterium of claim 1 wherein the bacterium comprises at least five of nucleic acid molecules (a)-(bs).

6. The bacterium of claim 1 wherein at least one of the polypeptides is heterologous to the bacterium.

7. The bacterium of claim 1 wherein at least two of the polypeptides are heterologous to the bacterium.

8. The bacterium claim 1 wherein the bacterium is a Corynebacterium glutamicum bacterium.

9. The bacterium of claim 1, wherein the bacterium comprises (aj) and (ak).

10. The bacterium of claim 1, wherein the bacterium comprises (r), (s) and (t).

11. The bacterium of claim 1, wherein the bacterium comprises (a), (b) and (c).

12. The bacterium of claim 1, wherein the bacterium comprises (d) and (e).

13. The bacterium of claim 1, wherein the bacterium comprises (i) and (j).

14. The bacterium of claim 1, wherein the bacterium comprises (l) and (o).

15. The bacterium of claim 1, wherein the bacterium comprises (p) and (q).

16. The bacterium of claim 1, wherein the bacterium comprises (bi), (bj), and (bk).

17. The bacterium of claim 1, wherein the bacterium comprises (bi), (bj), (bk) and (bl).

18. The bacterium of claim 1, wherein the bacterium comprises (bi), (bj), (bk) and at least one of: (1) (bm) or (2) (bn) and (o).

19. The bacterium of claim 1, wherein the bacterium comprises (bi), (bj), (bk) (bl) and at least one of: (1) (bm) or (2) (bn) and (bo).

20. A method of producing an amino acid or a related metabolite, the method comprising:
cultivating the bacterium claim 1 under conditions that allow the amino acid or the related metabolite to be produced, and collecting a composition that comprises the amino acid or related metabolite from the culture.

21. The method of claim 1 wherein the amino acid is selected from: methionine, S-adenosylmethionine, lysine, theronine and cysteine.

22. The bacterium of claim 1 comprising at least one isolated nucleic acid molecule selected from the group consisting of (a)-(an) and at least one isolated nucleic acid molecule selected from the group consisting of (ao)-(bs).

23. The bacterium of claim 1 comprising at least one isolated nucleic acid molecule selected from the group consisting of (a)-(an) and at least two isolated nucleic acid molecules selected from the group consisting of (ao)-(bs).

24. The bacterium of claim 1 comprising at least two isolated nucleic acid molecules selected from the group consisting of (a)-(an) and at least one isolated nucleic acid molecule selected from the group consisting of (ao)-(bs).

25. The bacterium of claim 1 comprising at least two isolated nucleic acid molecules selected from the group consisting of (a)-(an) and at least two isolatd nucleic acid molecules selected from the group consisting of (ao)-(bs).

26. The bacterium of claim 1 comprising: an isolated nucleic acid molecule encoding a variant aspartokinase with reduced feedback inhibition, a variant homoserine dehydrogenase with reduced feedback inhibition or a variant O-acetylhomoserine sulfhydrylase with reduced feedback inhibition.
